(19) United States
(12) Patent Application Publication
(10) Pub. No.: US 2023/0203157 A1
(43) Pub. Date: Jun. 29, 2023

(54) BISPECIFIC MOLECULES FOR SELECTIVELY MODULATING T CELLS

(71) Applicant: Dana-Farber Cancer Institute, Inc., Boston, MA (US)

(72) Inventors: James Torchia, Boston, MA (US); Gordon J. Freeman, Brookline, MA (US)

(21) Appl. No.: 17/927,529

(22) PCT Filed: May 27, 2021
(86) PCT No.: PCT/US2021/034526

§ 371 Date: Nov. 23, 2022

Related U.S. Application Data
(60) Provisional application No. 63/030,714, filed on May 27, 2020.

(51) Int. Cl. C07K 16/28; C07K 16/30; C12N 15/63

(52) U.S. Cl. CPC C07K 16/2803; C07K 16/30; C12N 15/63; C07K 2317/31; C07K 2317/52; C07K 2317/92

(57) ABSTRACT

The present invention relates, in part, to methods for inhibiting activation of T cells in a cell-specific and/or tissue-specific manner, and to bispecific molecules comprising a first part that specifically binds to PD-1 and a second part that specifically binds to a surface antigen of a target cell other than a T cell.

### CROSS-REFERENCE TO RELATED APPLICATIONS

This application claims the benefit of priority to U.S. Provisional Application Serial No. 63/030,714, filed on 27 May 2020; the entire contents of said application is incorporated herein in its entirety by this reference.

### STATEMENT OF RIGHTS

This invention was made with government support under grant number P01 AI056299 awarded by the National Institutes of Health. The government has certain rights in the invention.

### BACKGROUND OF THE INVENTION

Despite significant advances in various immunotherapies, serious complications continue to present themselves in various contexts. For example, when one receives a solid organ transplantation, systemic immunosuppressants can be used, but they put patients at risk for infection, cancer, and drug toxicity. As another example, autoimmune diseases can also be treated to some extent with systemic immunosuppressants, but these again put patients at risk for infection, cancer, and drug toxicity.

In the treatment of some types of cancers, for example leukemia, one is often confronted with graft-versus-host disease (GVHD). After bone marrow transplantation, both GVHD and graft-versus leukemia (GVL) are mediated by allogeneic lymphocytes. Agents that suppress GVHD also suppress GVL. Also, in the treatment of some types of cancers, checkpoint-inhibitor immune related adverse effects (irAE) are observed. Treatment of irAE with high dose steroids or other immunosuppressants can dampen antitumor immunity. Again, in the treatment of some types of cancers, for example using engineered immune cell therapy, one is often confronted with “on-target, off-tumor” toxicity.

Therefore, there is a need in the field for more advanced immunotherapies and for agents and methods that reduce the complications observed with different immunotherapies.

### SUMMARY OF THE INVENTION

The present invention is based, at least in part, on the discovery that soluble PD-1 antibodies are not capable of activating PD-1, but that antibodies against PD-1 on a surface can function as an agonist, and that co-localization of PD-1 stimulus with TCR stimulus is necessary for T cell inhibition, suggesting that there are two requirements for PD-1 activation: clustering and co-localization. Agents have been generated and described herein that are capable of selectively inhibiting T cell activation in the context of desired cells, cell types, and/or tissues, while permitting T cell activation elsewhere.

In some uses of the methods disclosed herein, it is disadvantageous to block the binding of PD-1 with its ligands, PD-L1 and/or PD-L2. In such methods, “non-blocking” anti-PD-1 clones are preferred and used in the relevant embodiments. In other methods disclosed herein, it is advantageous to block the binding of PD-1 with its ligands. In these methods “blocking” clones are preferred.

In some embodiments, the disclosed bispecific molecules function as an adapter: allowing the presence of a surface antigen to govern PD-1 clustering. Therefore, a higher surface antigen density will likely result in more robust PD-1 clustering to mediate PD-1 agonism and T cell activity inhibition.

In some aspects, bispecific molecules including a first part that specifically binds to PD-1 and a second part that specifically binds to a surface antigen of a target cell other than a T cell, optionally wherein the bispecific molecule binds to PD-1 on a T cell are disclosed. The simultaneous binding of the bispecific molecule to the surface antigen and to PD-1 (1) facilitates clustering of PD-1, such as in proximity to an immunological synapse; (2) reduces or prevents T cell-toxicity directed to the target cell; or (3) both facilitates clustering of PD-1, such as in proximity to an immunological synapse, and/or reduces or prevents T cell-activity (such as inhibition of intracellular interferon gamma production, cytokine secretion, T cell proliferation, and the like), and/or toxicity directed to the target cell, optionally wherein i) the bispecific molecule binds to PD-1 on a T cell; ii) T cell activity is cytokine production and/or T cell proliferation; iii) at least the first part or the second part comprises a monoclonal or polyclonal antibody, or an antigen-biding fragment thereof; iv) the bispecific molecule has a single binding site for PD-1; and/or v) the bispecific molecule has a single binding site for PD-1 and a single binding site for the surface antigen.

Numerous embodiments are further provided that may be applied to any aspect of the present invention and/or combined with any other embodiment described herein. For example, in some embodiments, the difference in the K_{d} of binding to PD-1 and the K_{d} of binding to the tissue-specific surface antigen is at least 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, 40-, 41-, 42-, 43-, 44-, 45-, 46-, 47-, 48-, 49-, 50-, 55-, 60-, 65-, 70-, 75-, 80-, 85-, 90-, 95-, 100-, 150-, 200-, 250-, 300-, 350-, 400-, 450-, 500-, 600-, 700-, 800-, 900-, 1,000-, 2,000-, 3,000-, 4,000-, 5,000-, 6,000-, 7,000-, 8,000-, 9,000-, 10,000-fold, or more, or any range in between, inclusive, such as 15- to 50-fold, optionally further wherein the K_{d} of binding to PD-1 is less than the K_{d} of binding to the tissue-specific surface antigen. In some embodiments, the difference in the off-rate when binding to PD-1 and the off-rate when binding to the tissue-specific surface antigen is at least 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, 40-, 41-, 42-, 43-, 44-, 45-, 46-, 47-, 48-, 49-, 50-, 55-, 60-, 65-, 70-, 75-, 80-, 85-, 90-, 95-, 100-, 150-, 200-, 250-, 300-, 350-, 400-, 450-, 500-, 600-, 700-, 800-, 900-, 1,000-, 2,000-, 3,000-, 4,000-, 5,000-, 6,000-, 7,000-, 8,000-, 9,000-, 10,000-fold, or more, or any range in between, inclusive, such as 15- to 50-fold, optionally further wherein the off-rate of binding to PD-1 is slower than the off-rate of binding to the tissue-specific surface antigen. In some embodiments, the first part specifically binds to PD-1 at a site different from the PD-L1 binding site of PD-1. In some embodiments, the first part blocks binding of neither PD-L1 nor PD-L2 to PD-1. In some such embodiments, the bispecific molecule acts as an agonist of PD-1 at a tissue expressing the surface antigen, but not at a tissue not expressing the surface antigen. In other embodiments, the first part i) specifically binds to PD-1 and blocks the binding of PD-1 with PD-L1 and/or PD-L2 (e.g., such as by binding PD-1 and causing steric hindrance for binding of PD-L1 and/or PD-L2) and/or ii) specifically binds to PD-1 at the PD-L1 and/or PD-L2 binding site of PD-1, such as to block the binding of PD-1 with PD-L1 and/or PD-L2. In some such embodiments, the bispecific molecule acts as an agonist of PD-1 at a tissue expressing the surface antigen, and acts as an antagonist of PD-1 at a tissue not expressing the surface antigen. For example, such a bispecific molecule may be used both as an immune checkpoint inhibitor to treat cancer, as well as a treatment or prophylactic against immune-related adverse events (irAE), such as in a first-line setting as an immune checkpoint inhibitor that carries a lower risk of irAE. For any embodiment described herein, it is contemplated that recitations of percent homology apply to any binding protein, such as a variable sequence of a binding protein and/or a CDR sequence thereof, such as one, two, or three light chain CDR (CDR-L1, CDR-L2, and/or CDR-L3) sequences and/or one, two, or three heavy chain CDR (CDR-H1, CDR-H2, and/or CDR-H3) sequences thereof, such as those listed in Table 4, described elsewhere herein, or otherwise well-known in the art. For example, in certain embodiments, the first part includes an amino acid sequence having at least 60%, 65%, 70%, 75%, 80%, 85%, 90%, or 95% sequence identity with an amino acid sequence selected from the group consisting of sequences listed in Table 1, Table 4A, and Table 4B, optionally wherein the sequence is SEQ ID NO: 30. In certain embodiments, the first part includes an amino acid sequence having at least 60%, 65%, 70%, 75%, 80%, 85%, 90%, or 95% sequence identity with SEQ ID NO: 31. In certain embodiments, the first part includes an amino acid sequence having at least 60%, 65%, 70%, 75%, 80%, 85%, 90%, or 95% sequence identity with an extracellular domain of PD-L1 or PD-L2, optionally wherein the target is a receptor for the extracellular domain and/or the extracellular domain of PD-L1 has the sequence of SEQ ID NO: 24. In some embodiments, the surface antigen is selected from the group of surface antigens listed in Table 3A, Table 3B, and Table 3C, or an MHC class I allele or MHC class II allele, optionally wherein the allele is an HLA allele or wherein the MHC allele is H-2Kb. In certain embodiments, the second part includes an amino acid sequence having at least 60%, 65%, 70%, 75%, 80%, 85%, 90%, or 95% sequence identity with SEQ ID NO: 21. In certain embodiments, the second part includes an amino acid sequence having at least 60%, 65%, 70%, 75%, 80%, 85%, 90%, or 95% sequence identity with SEQ ID NO: 22. In certain embodiments, the bispecific molecule includes an amino acid sequence having at least 60%, 65%, 70%, 75%, 80%, 85%, 90%, or 95% sequence identity with any one among SEQ ID NOs: 1, 2, 3, 4, 5, 6, 7, 8, 9, 10, 11, 12, 13, and 14. In some embodiments, at least the first part or the second part is a chimeric, humanized, composite, murine, or human monoclonal or polyclonal antibody, or antigen-binding fragment thereof. In some embodiments, at least the first part or the second part is detectably labeled, includes an effector domain, includes an Fc domain, and/or is selected from the group consisting of Fv, Fav, F(ab′)2), Fab′, dsFv, scFv, sc(Fv)2, and diabodies fragments.

In some aspects, isolated nucleic acid molecules are disclosed that encode a bispecific molecule described herein. The isolated nucleic acid molecule may take a number of forms as described further herein, such as RNA, DNA, cDNA, stabilized mRNA, and the like, optionally encoding heterologous sequences and/or comprising additional nucleic acid sequences. In some embodiments, the isolated nucleic acid molecule is one that hybridizes, under stringent conditions, with the complement of a nucleic acid encoding a polypeptide selected from the group consisting of the sequences of SEQ ID NOs: 1-14, 21-26, 28, and 30-31, or a sequence with at least about 95% homology to a nucleic acid encoding a polypeptide selected from the group consisting of the sequences of SEQ ID NOs: 1-14, 21-26, 28, and 30-31. In some aspects, vectors are disclosed, which include such isolated nucleic acids. In some aspects, host cells are disclosed, which include such isolated nucleic acids, vectors, or which express the bispecific molecules disclosed herein, optionally wherein the host cell is genomically engineered and/or is a T cell that expresses a heterologous protein such as a chimeric antigen receptor (CAR).. In some aspects, devices or kits are disclosed, which include at least one bispecific molecule as disclosed, said device or kit optionally including a label to detect the at least one bispecific molecule or a complex including the bispecific molecule. In some aspects, methods of producing at least one bispecific molecule include: (i) culturing a transformed host cell which has been transformed by a nucleic acid including a sequence encoding at least one bispecific molecule as disclosed under conditions suitable to allow expression of said bispecific molecule; and (ii) recovering the expressed bispecific molecule.

In certain aspects, methods of inhibiting activation of T cells in a cell-specific and/or tissue-specific manner in a sample include contacting the sample with the bispecific molecule of any of the described embodiments herein, optionally wherein the subject is at risk of, suspected of having, or afflicted with a cancer to thereby treat the cancer, are disclosed. In some embodiments of such methods, the bispecific molecule inhibits activation of T cells interacting with the target cell when the bispecific molecule simultaneously binds to the surface antigen and to PD-1.

In some aspects, methods of inhibiting activation of T cells in a cell-specific and/or tissue-specific manner in a subject include administering to the subject the bispecific molecule of any of the described embodiments herein are disclosed. In some embodiments of such methods, the bispecific molecule inhibits activation of T cells interacting with the target cell when the bispecific molecule simultaneously binds to the surface antigen and to PD-1.

In some embodiments, the compositions described herein are useful for prophylaxis and/or treatment of a wide variety of conditions, such as cancer. In some embodiment, blocking LoSTIMs are useful for treating cancer and both blocking and non-blocking LoSTIMs are useful for ameliorating checkpoint-inhibitor immune related adverse events (irAE). In some embodiments, the subject is set to receive or has received solid organ transplantation, in which case the surface antigen may be a mismatched MHC class I or class II. In some embodiments, the subject is at risk of, suspected of having, or afflicted with an autoimmune disease to thereby treat the autoimmune disease, in which case the surface antigen may be one that is expressed on pathologically inflamed tissue. In some embodiments, the subject is at risk of, suspected of having, or afflicted with graft-versus-host disease (GVHD) to thereby treat the GVHD, in which case the surface antigen may be one that is expressed on inflamed tissue but not on cancerous cells. For example, the subject may be set to receive or has received an allogeneic bone marrow transplantation, in which case the surface antigen may be one that is expressed on tissues that are commonly affected by GVHD. In some embodiments, the subject at risk of, suspected of having, or afflicted with from checkpoint-inhibitor immune related adverse events (irAE) to thereby treat the irAE, in which case the surface antigen may be one that is expressed on inflamed tissue but not on cancerous cells. For example, compositions described herein may be used as prophylaxis against immune related adverse effects, in which case the surface antigen would be expressed on tissues commonly affected by immune related adverse effects. In some embodiments, the subject is set to receive or has received an engineered immune cell therapy, in which case the surface antigen may be one that is expressed in normal tissues that also express a tumor-associated antigen targeted by the immune cell therapy, but is one that is not expressed on cancerous cells.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1A - FIG. 1C show an overview of exemplary LoSTIMs (i.e., bispecific molecules, as described and used in this disclosure). FIG. 1A shows a schematic representation of a LoSTIM. A LoSTIM is a molecule capable of simultaneously binding to PD-1 as well as to an arbitrary cell-specific and/or tissue-specific surface antigen expressed on tissues where inhibition of T cell activity is desired. FIG. 1B shows that an embodiment of a LoSTIM simultaneously binds PD-1 molecules on a T cell as well as molecules of an antigen on the surface of an opposing somatic cell, thereby clustering PD-1 on the T cell surface in proximity to an immunologic synapse between the two cells. This clustering and co-localization of PD-1 inhibits T cell activation. FIG. 1C shows that an embodiment of a LoSTIM binds PD-1 on the T cell but does not bind an antigen on the opposing somatic cell surface since its cognate antigen is not expressed by that cell. Because the LoSTIM is not fixed in place by binding to the opposing cell surface, it is not able to cluster PD-1 or co-localize PD-1 with the immunologic synapse on the T cell surface and therefore does not inhibit T cell activation. In this circumstance, if the PD-1 binding domain of the LoSTIM inhibits the engagement of PD-1 by its natural ligands, the LoSTIM acts as a potentiator of T cell activation.

FIG. 2 shows representative schematics of LoSTIMs used in some of the studies (e.g., those of Example 1). Four generalized LoSTIM designs were evaluated in the subsequent studies: (1) a PDL1-scFv fusion protein in which the scFv binds to a cell-specific and/or tissue-specific surface antigen expressed on tissues where T cell inhibition is desired; (2) a PDL1-mAb fusion in which the mAb is directed against a cell-specific and/or tissue-specific surface antigen expressed on tissues where T cell inhibition is desired; and (3) & (4), bispecific monoclonal antibodies with avidity for PD-1 and for a cell-specific and/or tissue-specific surface antigen expressed on tissues where T cell inhibition is desired. LoSTIMs 1 through 4 have bispecific avidity for Thy1.2 (CD90.2) and mouse PD-1. The anti-Thy 1.2 binding domain is the variable region of the anti-Thy 1.2 clone AF6 and the anti-PD-1 binding domain is via the variable region of anti-PD-1 clone 29F.1A12 (1A12). LoSTIMs 5 through 8 have bispecific avidity for H-2Kb (a C57B6 class I MHC allele) and mouse PD-1. The anti-H-2Kb binding domain is the variable region of the anti-H-2Kb clone AF6-88.5.3 (AF6). It should be noted that the anti-PD-1 clone 1A12 is known to block the interaction of PD-1 with its natural ligands and function as a PD-1 antagonist. The CH1, CH2, and CH3 domains of the antibody LoSTIMs contain several point mutations known to inhibit C1q and Fc gamma receptor binding, thus rendering these Fc domains “inert.” The sequences of these LoSTIMs are presented in an appendix at the end of this document.

FIG. 3A - FIG. 3D show LoSTIM binding results. FIG. 3A shows experimental results in which 300-mPD-1 cells, transgenic mouse pro-B cells which overexpress mouse PD-1, were exposed to varying concentrations of LoSTIMs 2-4 and 6-8. Binding was detected via an anti-mIgG2a-PE conjugate. FIG. 3B shows data from FIG. 3A normalized to allow for a better assessment of relative PD-1 avidities. FIG. 3C shows experimental results in which bone-marrow-derived dendritic cells (BMDCC) generated from C57B6 bone marrow cells cultured with GM-CSF for 8 days were exposed to varying concentrations of LoSTIMs 6-8. Binding was detected via an anti-mIgG2a-PE conjugate. FIG. 3D shows experimental results in which BMDDC were exposed to varying concentrations of LoSTIMs 6-8. Unbound LoSTIM was first washed away and then bispecific binding was assessed by detecting the binding of a mouse PD-1-human Fc fusion protein. An anti-human IgG-PE conjugate was used as the detector. This demonstrated that the LoSTIMs were capable of binding both H-2Kb and PD-1 simultaneously.

FIG. 4 shows that OT-I T cell activation by SIINFEKL-pulsed bone-marrow-derived dendritic cells (BMDDC) is inhibited by LoSTIMs. C57B6 bone marrow cells were cultured with GM-CSF for 8 days to yield BMDDC. These BMDDC were pulsed with 10 micromolar of Ser-Ile-Ile-Asn-Phe-Glu-Lys-Leu (SIINFEKL) peptide for 6 hours, washed, then co-cultured with CFSE-labeled OT-I CD8a+ T cells, which express a recombinant TCR that recognizes SIINFEKL in the context of H-2Kb. These T cells were obtained via negative magnetic selection from OT-I mouse splenocytes. The co-cultures were treated with 0.5 micromolar of the anti-H-2Kb-directed LoSTIMs 5, 6, 7, or 8. Cells were co-cultured for 72 hours. The histograms presented above represent populations gated on CD8+ cells. OT-I T cells cultured without BMDDC were inactive (1.4% proliferation by CFSE dilution). OT-I T cells stimulated with BMDDC were activated (87.3% proliferation by CFSE dilution). LoSTIM 5 had negligible effect on the activation OT-I T cells by BMDDC (84.3% proliferation by CFSE dilution). LoSTIM 6 inhibited OT-I T cell activation by BMDDC (62.4% proliferation by CFSE dilution). LoSTIM 7 inhibited OT-I T cell activation by BMDDC most potently (43.9% proliferation by CFSE dilution). LoSTIM 8 had an effect on OT-I T cell activation (78.4% proliferation by CFSE dilution).

FIG. 5 shows that LoSTIM-mediated inhibition of OT-I T cell activation is not due to H-2Kb blockade and requires a LoSTIM that can engage PD-1. To address whether inhibition of OT-I activation by SIINFEKL-pulsed BMDDC was simply due to blockade of H-2Kb antigen presentation by LoSTIMs, CFSE-labeled OT-I CD8a+ T cells were cocultured with BMDDC in the presence of 0.5 micromolar of anti-H-2Kb, clone AF6. The H-2Kb binding domains of LoSTIMs 5-8 are derived from this clone, so AF6 would be expected to bind the same epitope of H-2Kb as LoSTIMs 5-8. After 57 hours of co-culture, OT-I T cell activation was assessed by CFSE dilution and CD69 expression. OT-I cells co-cultured with BMDDC in the absence of antibody treatment were activated (63.6% proliferation by CFSE dilution and 42.6% CD69 expression). The addition of anti-H-2Kb clone AF6 to such a co-culture did not inhibit T cell activation (73.4% proliferation by CFSE dilution and 67.4% CD69 expression). Addition of equimolar quantities of anti-H-2Kb clone AF6 and LoSTIM 7 to such a co-culture did result in some inhibition of T cell activation (42.5% proliferation by CFSE dilution and 38.8% CD69 expression).

FIG. 6 shows that allogeneic T cell activation is inhibited by LoSTIM treatment. To address whether LoSTIM molecules can inhibit the activation of T cells by allogeneic cells, C57B6 BMDDC were co-cultured with CFSE-labeled BALB/c CD8 T cells for 84 hours in the presence of LoSTIMs 5-8 at 0.5 micromolar. BALB/c CD8 T cells co-cultured with BMDDC derived from C57B6 mice were potently activated (91.8% proliferation by CFSE dilution). Treatment of such a co-culture with LoSTIM 5 had only a minimal inhibitory effect on T cell activation (85.5% proliferation by CFSE dilution). Treatment with LoSTIM 6 inhibited T cell activation (71.1% proliferation by CFSE dilution). Treatment with LoSTIM 7 resulted in the most potent inhibition of T cell activation (63.6% proliferation by CFSE dilution). Treatment with LoSTIM 8 also resulted in inhibition of T cell activation (69.1% proliferation by CFSE dilution).

FIG. 7A - FIG. 7B show that a LoSTIM binds the surface of an interacting cell to inhibit T cell activation, and that a LoSTIM that inhibits the binding of PD-1 to its natural ligands will potentiate T cell activation if it does not also bind the surface of the interacting cell. To determine whether binding of a LoSTIM to the surface of an opposing cell is necessary for the inhibition of T cell activation, CFSE-labeled OT-I CD8 T cells were co-cultured with SIINFEKL-pulsed BMDDC in the presence of LoSTIMs 1-4 at 0.5 micromolar for 57 hours (FIGS. 7A and 7B). LoSTIMs 1-4 are identical in molecular design to LoSTIMs 5-8 except that they carry a Thy1.2 (CD90.2) binding domain instead of an H-2Kb binding domain. Since BMDDCs do not express Thy1.2 (panel B above), LoSTIMs 1-4 will not bind the surface of the BMDDCs. These LoSTIMs carry the same PD-1 binding domain as LoSTIMs 5-8 and, therefore, they will still bind PD-1 on T cells. OT-I cells co-cultured with BMDDC in the absence of antibody treatment were activated (63.6% proliferation by CFSE dilution and 42.6% CD69 expression). Treatment with LoSTIMs 1-4 did not inhibit T cell activation in these co-cultures. In fact, LoSTIMs 1-4 potentiated T cell activation to varying degrees. LoSTIM 1 had a minimal potentiating effect on T cell activation (68.4% proliferation and 56% CD69 expression). LoSTIM 2 markedly potentiated T cell activation (98.6% proliferation and 78.9% CD69 expression). LoSTIM 3 also potentiated T cell activation (90.1% proliferation and 61% CD69 expression). LoSTIM 4 also markedly potentiated T cell activation (96.1% proliferation and 79% CD69 expression).

FIG. 8 shows a representative conceptual overview. The trans bispecific binding of a LoSTIM to PD-1 on the T cell and to a cell-surface antigen on the opposing target cell results in PD-1 clustering on the T cell surface and co-localization of this cluster with the immunological synapse, thereby activating PD-1 and inhibiting T cell activity. PD-1 is free to move laterally on the T cell surface and it is activated when clustered in proximity to the immunological synapse. Thus, trans bispecific binding of the LoSTIM to the surface antigen on the target cell and to PD-1 on the T cell will recruit and cluster PD-1 at the cell-cell interface, where the immunological synapse is located. This activates PD-1, resulting in inhibition of TCR signaling and T cell activity. Engagement of the antigen on the target cell surface is necessary for a LoSTIM to function as a PD-1 agonist: simply binding PD-1 alone will not cluster and co-localize PD-1 to the immunological synapse and will therefore not inhibit T cell activity. This property allows a LoSTIM to function as selective PD-1 agonist, inhibiting T cell activity only in tissues that express the surface antigen. A LoSTIM may or may not be designed to block the engagement of PD-1 by its natural ligands. If it is designed to block the engagement of PD-1 by its ligands, it will act as an antagonist of PD-1 on T cells interacting with tissues that do not express the surface antigen, while still acting as an agonist of PD-1 on T cells interacting with tissues that do express the surface antigen. A single LoSTIM molecule of this design will therefore function as either an inhibitor or potentiator of T cell activity depending on the tissue context.

FIG. 9 shows binding to cells that express a model surface antigen H-2K^{b}. Binding to H-2K^{b} was assessed by flow cytometry using the C57BL/6 colorectal cancer cell line MC38, which is known to express H-2K^{b}. Specificity was demonstrated by assessing binding to CT-26 cells, a BALB/c colorectal cancer cell line known not to express H-2Kb. The LoSTIM bound avidly to MC38 cells but not to CT-26 cells. In a separate experiment, binding to H-2K^{b} was assessed by flow cytometry using BMDDC from C57BL/6 mice, which express H-2K^{b}, or BMDDC from C3H mice, which do not express H-2K^{b}. The LoSTIM bound avidly to C57BL/6 BMDDC cells but not to C3H BMDDC cells.

FIG. 10 shows binding to PD-1 and bispecific binding to PD-1 and H-2K^{b}. Binding of the LoSTIM to PD-1 was assessed by flow cytometry using 300-mPD-1 cells, transgenic mouse pro-B cells that stably express mouse PD-1. The LoSTIM bound 300-mPD-1 cells avidly. Bispecific binding to PD-1 and H-2K^{b} was assessed by flow cytometry using MC38 cells, which express H-2K^{b} but not PD-1, and a soluble mouse PD-1-Fc fusion bearing a human IgG1 Fc domain (mPD-1-hFc). Unbound LoSTIM was first washed away before the cells were exposed to the mPD-1-hFc fusion. Binding of the mPD-1-hFc to MC38 cells as measured by a fluorophore-labeled an anti-human IgG secondary antibody indicated that the LoSTIM was capable of binding to both H-2K^{b} on MC38 cells and to the mPD-1-hFc simultaneously. This also confirmed the specificity of PD-1 binding.

FIG. 11 shows binding to T cells that express PD-1, but not to T cells that do not express PD-1. To further confirm PD-1 binding specificity, binding of the LoSTIM to activated primary T cells that express PD-1 was compared with binding to naive primary T cells that do not express PD-1. These T cells were obtained from mice from BALB/cByJ mice that do not express H-2K^{b}, therefore binding to activated T cells should only occur if the LoSTIM binds specifically to PD-1. The LoSTIM bound avidly to activated BALB/cByJ T cells but not to naive BALB/cByJ T cells, further confirming the specificity of PD-1 binding.

FIG. 12 shows inhibition of T cell activation. To assess the effect of the LoSTIM on T cell activation by APCs that express the H-2K^{b} model surface antigen, primary CD4+ BALB/cByJ T cells (H-2K^{b} negative) were co-cultured with B-cells obtained from C57BL/6 mice (H-2K^{b} positive) at a 1:1 ratio for five days. Cultures were treated with 500 nanomolar of either LoSTIM, the parent anti-H-2K^{b} antibody (AF6-88.5.3), the parent anti-PD-1 antibody (29F.1A12), or vehicle (PBS). The LoSTIM completely inhibited APC-induced proliferation of T cells as measured by CFSE dilution. Neither AF6-88.5.3, 29F.1A12, nor vehicle affected T cell proliferation. This demonstrates that the LoSTIM inhibits T cell activation by APCs that express a surface antigen that binds the LoSTIM. The bispecific binding of the LoSTIM to PD-1 on the T cell and to the surface antigen on the target cell was necessary for this effect, as demonstrated by a lack of T cell inhibition by either AF6-88.5.3, which binds the model surface antigen H-2K^{b}, or 29F.1A12, which binds PD-1. In a similar separate experiment, two concentrations of LoSTIM were used and the inhibition of T cell proliferation was found to be dose-dependent.

FIG. 13 shows inhibition of TCR signaling. To assess the effect on TCR signaling, CD8+ T cells expressing a transgenic TCR that recognizes the ovalbumin antigen SIINFEKL were purified from OT-1 mice, activated by plate-bound anti-CD3/CD28 antibody, rested, and then re-stimulated by C57BL/6 cells loaded with the SIINFEKL antigen. TCR activation was assessed by phosphoflow cytometry using a fluorophore-labeled antibody specific for phosphorylated Zap70. Zap70 is a membrane-proximal kinase that is phosphorylated and activated upon TCR activation and is responsible for TCR signal transduction. PD-1 activation is known to inhibit TCR-mediated Zap70 phosphorylation. Treatment with LoSTIM inhibited antigen-induced Zap70 phosphorylation.

FIG. 14 shows cell-specific and/or tissue-specific inhibition of T cell activation. The effect of the LoSTIM was compared when T cells were co-cultured with APCs that express the model cell surface antigen H-2K^{b} and when T cells were co-cultured with APCs that do not express H-2K^{b}. To accomplish this, purified BALB/cByJ CD8+ T cells were co-cultured with either BMDDC from C57BL/6 mice, which express the model cell surface antigen H-2K^{b}, or BMDDC from C3H mice, which do not express H-2K^{b}. Cultures were treated with varying concentrations of LoSTIM. Relative T cell proliferation was calculated by normalizing the percentage T cell proliferation by the percentage T cell proliferation of co-cultures treated with vehicle (PBS). Because the strength of allostimulation can vary between backgrounds, this calculation was performed separately for BALB/cByJ T cells stimulated by C57BL/6 BMDDC and C3H BMDDC. T cell activation was inhibited when the LoSTIM bound the target cell but did was not inhibited when the LoSTIM did not bind the target cell.

FIG. 15 shows inhibition of allorejection in vivo. A model of allorejection was used to assess the ability of the LoSTIM to inhibit immune activity in vivo against target cells that express an antigen that binds the LoSTIM. MC38 cells (of C57BL/6 origin) were injected subcutaneously in BALB/cByJ mice. Normally, the MC38 tumor will be rejected in the BALB/cByJ mice by an alloresponse to its different MHC. Increased tumor growth indicates that this alloresponse is ameliorated. Tumor volumes were significantly larger in mice that received intraperitoneal injections of LoSTIM when compared with mice that received PBS as a vehicle control (p = 0.0297). This demonstrates that the LoSTIM can inhibit immune activity against target cells in vivo, if the target cells express an antigen to which the LoSTIM binds. Note that purified LoSTIM monomer (discussed in FIG. 17 below) was used for this experiment.

FIG. 16 shows treatment of syngeneic tumor in vivo. A model of syngeneic tumor treatment was used to assess the in vivo effect of the LoSTIM on immune activity against target cells that do not express an antigen that binds the LoSTIM. CT-26 cells were injected subcutaneously in BALB/cByJ mice and mice were treated with either LoSTIM, 29F.1A12 (the parent anti-PD-1 antibody used in design of the LoSTIM), or PBS as a vehicle control. The LoSTIM did not inhibit immune activity against CT-26 cells. Instead, the LoSTIM promoted immune activity against CT-26 cells, functioning similarly to the PD-1 inhibitor 29F.1A12. This demonstrates that the LoSTIM will not inhibit immune activity against target cells that do not express an antigen to which it binds. Moreover, it demonstrates that a LoSTIM that blocks the natural ligation of PD-1 will function as a PD-1 inhibitor in these contexts and can treat tumor. Note that purified LoSTIM monomer (discussed in FIG. 17 below) was used for this experiment.

FIG. 17 shows size exclusion ultra-performance liquid chromatography (SE-UPLC) analysis of LoSTIM 8, which was used in experiments presented in FIGS. 3, 4, 5, 6, 7, 10, 12, and 14 (upper left panel). 89% of the species are 220 kDa in size, the approximate expected molecular weight of LoSTIM 8, and 11% of the species have larger molecular weights, representing multimers (pentamers and trimers) of the LoSTIM (right panels). After preparative size-exclusion chromatography was performed, SE-UPLC analysis confirmed that 85% of the multimers were removed and 98.4% of the remaining species were LoSTIM monomer (lower left panel).

FIG. 18 shows the effect of purified LoSTIM monomer and purified multimers on T-cell activation. To assess the effect of each molecular species on T cell activation, primary CD4+ BALB/cByJ T cells (H-2K^{b} negative) were co-cultured with B-cells obtained from C57BL/6 mice (H-2K^{b} positive) at a 1:1 ratio for five days. Cultures were treated with indicated concentrations of either LoSTIM that was not purified or LoSTIM monomer that was purified by preparative size-exclusion chromatography. Unpurified LoSTIM inhibited T-cell proliferation at 250 nM and 500 nM however, unexpectedly, purified LoSTIM monomer did not inhibit T-cell proliferation at these concentrations. This indicates that T-cell inhibition at these concentrations was due to the multimers. Consistent with this indication, purified high molecular weight multimers (HMW 1 or HMW 2) were capable of potently inhibiting T-cell proliferation at approximately the concentration (18 nM - 33 nM) that they were present in 250 nM of unpurified LoSTIM that was comprised of 11% multimers. It will also be appreciated that the larger HMW 1 species appeared to inhibit T-cell proliferation more potently than HMW 2, which may be due to a larger number of PD-1 binding sites resulting in more potent PD-1 clustering.

FIG. 19 shows tissue-specific inhibition of T cell activation by purified LoSTIM monomer. CFSE-labeled BALB/c CD8 T cells were cultured with BMDC from either C57BL/6 (H-2K^{b} positive) or C3H mice (H-2K^{b} negative) in the presence of varying concentrations of purified LoSTIM monomer. The LoSTIM potently inhibited T-cell activation by C57BL/6 target cells that bind the LoSTIM (H-2K^{b} positive) but did not inhibit T-cell activation by C3H target cells that do not bind the LoSTIM (H-2K^{b} negative). Unexpectedly, the inhibitory effect of the LoSTIM occurred maximally within a specific concentration range before it appeared to diminish as the concentration was further increased.

FIG. 20 shows inhibition of T-cell activation by purified LoSTIM monomer in another model system. CFSE-labeled OT-I CD8+ T-cells were co-cultured with SIINFEKL-pulsed BMDC from C57BL/6 mice in the presence of varying concentrations of purified LoSTIM monomer. CFSE mean fluorescence intensity (MFI) was used as a metric of T-cell proliferation and activation, with lower values indicating more T-cell proliferation. Again, the LoSTIM inhibited T-cell activation most potently in a specific concentration range, with the inhibitory effect becoming more pronounced as the concentration was increased before then becoming less pronounced. Surprisingly, the LoSTIM actually enhanced rather than inhibited T-cell activation at the highest concentration tested.

FIG. 21 shows that LoSTIM-mediated T-cell inhibition occurs at concentrations where binding to both of its cognate antigens is not saturated. As demonstrated in the preceding two figures and reproduced in the two panels on the right of this figure, the LoSTIM inhibited T-cell activation at 0.4 nM, 4 nM, and 40 nM in both model systems, whereas, at 400 nM, its inhibitory effect was diminished in the allogeneic T-cell activation model and was absent in the model antigen (SIINFEKL) model. In fact, T-cell activation by SIINFEKL-pulsed BMDC was enhanced, not inhibited, at the 400 nM concentration. Comparison can be made to binding curves for each of the LoSTIMs cognate antigens (PD-1 and H-2Kb) overlaid in the left panel of this figure, which were generated by measuring binding separately to H-2Kb+ C57BL/7 BMDC and to PD-1+ activated T-cells. Binding to the lowest affinity binding partner begins to approach saturation at around 100 nM and appears to be saturated around 1000 nM. This is the concentration range where the inhibitory effect of the LoSTIM is lost in both model systems. Binding must still occur to both binding partners for an inhibitory effect to occur, though, and thus a difference in binding affinity for the two binding partners is advantageous and a wider difference should yield a broader range of concentrations where the LoSTIM can inhibit T-cells. Therefore, a LoSTIM that has a larger difference in measured K_{d} between its binding partners would be expected to mediate T-cell inhibition over a wider range of concentrations than a LoSTIM where the K_{d} for each of the binding partners is more similar.

FIG. 22 shows that bispecific binding increases before decreasing sharply as LoSTIM concentration rises in an experimental model where competition between bispecific and monospecific binding is allowed. MC38 cells (H-2K^{b}-positive, PD-1 negative) were incubated with varying concentrations of LoSTIM in the presence of a fixed concentration of recombinant mouse PD-1 extracellular domain fused to human PD-1 (mPD-1-hFc). After thoroughly washing away any protein that did not bind to the cells, cells were stained with anti-human IgG-PE to detect mPD-1-hFc bound to the cells via bispecific LoSTIM binding. This experiment demonstrated that, when competition is allowed between monospecific binding to each binding partner and bispecific binding to both binding partners simultaneously, bispecific binding occurs over a limited concentration range. The consequences of this for LoSTIM-mediated T-cell inhibition are depicted in the schematic on the right of the figure. When the LoSTIM is present at a concentration where bispecific binding is favored (a concentration where both binding partners are not saturated), bispecific binding allows the presence of a tissue-specific surface antigen on a target cell to cluster PD-1 on a T-cell interacting with that cell, and therefore inhibit the T-cell. Monospecific binding does not allow for this, though, and if the LoSTIM is present at a concentration where monospecific binding is favored, such as at concentrations at or above where binding to each binding partner is saturated, the presence of a tissue-specific surface antigen will not result in PD-1 clustering and T-cell inhibition will not occur, even if the LoSTIM is capable of binding to that antigen. This is consistent with the results presented in the preceding three figures.

FIG. 23 shows that a bivalent monospecific anti-PD-1 antibody can function to enhance immune-mediated clearance of tumor even if it does not block the ligation of PD-1 by PD-L1 or PD-L2. A bivalent monospecific antibody has one binding partner/antigenic determinate and has two binding sites for that binding partner/antigenic determinate. An example of this is a standard IgG antibody. 5E12 is a standard IgG antibody that binds PD-1 but does not block the interaction between PD-1 and PD-L1 or PD-L2. The upper left panel of this figure demonstrates that 5E12 binds avidly to 300-mPD-1 cells. The left middle and lower panels confirm this by demonstrating that increasing concentrations of 5E12 do not block the binding of recombinant mouse PD-L1 or PD-L2 Fc fusions (mPD-L1-Fc or mPD-L2 Fc) to 300-mPD-1 cells. The right panels assess the effect of 5E12 antibody on tumor growth in the model of tumor allorejection that was used to assess the ability of a LoSTIM to inhibit immune cell activity in vivo presented in FIG. 15. MC38 cells (of C57BL/6 origin) were injected subcutaneously in BALB/cByJ mice. Normally, the MC38 tumor will be rejected in BALB/cByJ mice by an alloresponse to its different MHC, but treatment with LoSTIM results in significantly larger tumors and longer average persistence of tumor before rejection. Two additional experimental conditions are presented in this figure: (1) concurrent treatment with LoSTIM plus 5E12 antibody and (2) treatment with 5E12 antibody alone. Concurrent treatment with LoSTIM and 5E12 reduced the protective effect of the LoSTIM, resulting in lower average tumor volumes and faster tumor rejection. Treatment with 5E12 alone significantly enhanced tumor clearance relative to vehicle control to the extent that no tumor growth was measurable. This enhancement of tumor clearance might be expected with an anti-PD-1 antibody that blocks the ligation of PD-1 by PD-L1 and/or PD-L2 but it is unexpected with an anti-PD-1 antibody that does not block PD-1 ligation. It is believed that the 5E12 antibody is capable of binding two PD-1 molecules on the T-cell surface and stabilizing these molecules in some orientation or some distance with respect to one another that is incompatible with their activation or that inhibits their activation. A LoSTIM with a single binding site for PD-1, such as a monovalent bispecific LoSTIM, is believed to allow the use of anti-PD-1 clones, such as 5E12, that may be incompatible with PD-1 activation when present in a bivalent form.

### DETAILED DESCRIPTION OF THE INVENTION

The present invention is based, at least in part, on the discovery that bispecific molecules that bind PD-1 and a surface antigen on a target cell different from a T cell can function as PD-1 agonists in a cell-specific and/or tissue-specific manner.

Accordingly, the present invention provides, among various aspects, bispecific molecules that are PD-1 agonists in desired cell, cell type, and/or tissue contexts, and thereby inhibit activation of T cells in such contexts, while being neither agonists nor antagonists of T cells in other tissues. The present invention also provides, among various aspects, bispecific molecules that are PD-1 agonists in some contexts, and thereby inhibit activation of T cells in such contexts, while being antagonists of PD-1 in other contexts, thereby potentiating activation of T cells in these other contexts.

The present invention also provides, among various aspects, methods related to inhibiting activation of T cells in a cell-specific and/or tissue-specific manner. Such methods may be used in the prevention of solid organ graft rejection, treatment of autoimmune diseases, treatment of cancer, and treatment of graft-versus-host disease.

In various embodiments, bispecific molecules of four broad design categories are disclosed. A first category includes bispecific antibodies (including IgG, IgM, and IgA monoclonal antibodies or variants). If a bispecific antibody is used, the Fc domain may contain modifications to decrease its binding to Fcy receptors. A second category includes natural ligands for PD-1 fused to antibodies or scFv against the surface antigen on cells on tissues in the body where the inhibition of T cell activity is desired. If ligand is fused to an antibody, the Fc domain may contain modifications to decrease its binding to Fcy receptors. A third category includes natural ligands for the surface antigen on cells on tissues in the body where the inhibition of T cell activity is desired (if one exists) fused to an antibody or scFv against PD-1. If ligand is fused to an antibody, the Fc domain may contain modifications to decrease its binding to Fcy receptors. A fourth category includes natural ligands for PD-1 fused to a natural ligand for the surface antigen on cells on tissues in the body where the inhibition of T cell activity is desired (either a direct fusion or as an Fc fusion). If an Fc fusion is used, the Fc domain may contain modifications to decrease its binding to Fcy receptors.

For each of these categories, the bispecific molecules may have various features. For example, the interaction with PD-1 may or may not block its ligation by natural ligands. As another example, the valency of binding (number of binding sites) for each antigenic determinant (either PD-1 or surface antigen on cells on tissues in the body where the inhibition of T cell activity is desired) may be any of the following: (1) monovalent, when there is one antigen binding domain (either a FAB region, scFv, or a natural ligand) for a given antigenic determinant (PD-1 or surface antigen on cells on tissues in the body where the inhibition of T cell activity is desired); (2) bivalent, when there are two antigen binding domains for a given antigenic determinant; and (3) multivalent, when there are multiple antigen binding domains for a given antigenic determinant. In addition, the stoichiometric ratio of PD-1 to surface antigen binding may vary: the number of binding domains for PD-1 may be equal to the number of binding domains for the surface antigen; there may be more PD-1 binding domains than surface antigen binding domains (e.g., 1, 2, 3, 4, 5, 6, or more PD-1 binding domains than surface antigen binding domains); or there may be more surface antigen binding domains than PD-1 binding domains (e.g., 1, 2, 3, 4, 5, 6, or more surface antigen binding domains than PD-1 binding domains).

The bispecific molecules, when used in various methods, may be therapeutically administered in many forms. For example, they may be administered as a therapeutic protein. As another example, they may be administered as a nucleic acid encoding the therapeutic protein (e.g., stabilized mRNA, DNA in an appropriate vector, and the like). As another example, they may be in the form of cells that are engineered to express the therapeutic protein (e.g., a CAR-T cell that is transformed with a nucleic acid that encodes the therapeutic protein).

The disclosed bispecific molecules may function as pharmacologic PD-1 agonists capable of inhibiting T cell activation. In some embodiments, the bispecific molecules may function as systemic pharmacologic immunosuppressants that inhibit T cell activity on some tissues but not others as a consequence of their design. In some embodiments, the bispecific molecules may function as pharmacologic agents that may both inhibit or potentiate T cell activity in different tissue contexts as a consequence of their design. Some embodiments, allow fashioning a PD-1 binding domain of an antibody clone that is known to inhibit PD-1 as a pharmacologic agonist of PD-1. Such a pharmacologic agonist of PD-1 may inhibit T cell activity in certain tissue contexts, but exhibit either no activity or a potentiating effect on T cell activity in other tissue contexts.

The disclosed bispecific molecules may inhibit T cell activity in certain tissues or cell types while simultaneously either potentiating or having no effect on T cell activity in other tissues. Several potential applications of the disclosed bispecific molecules for the treatment of various conditions are further described in Section VI, titled Uses and Methods of the Invention.

### I. Definitions

The articles “a” and “an” are used herein to refer to one or to more than one (i.e., to at least one) of the grammatical object of the article. By way of example, “an element” means one element or more than one element.

The term “altered amount” of a marker refers to increased or decreased copy number of a marker and/or increased or decreased nucleic acid level of a particular marker gene or genes in a sample, as compared to that of the marker in a control sample. The term “altered amount” of a marker also includes an increased or decreased protein level of a marker in a sample, as compared to the protein level of the marker in a normal, control sample.

The term “altered activity” of a marker refers to an activity of a marker which is increased or decreased in a disease state, e.g., in a biological sample, as compared to the activity of the marker in a normal, control sample. Altered activity of a marker may be the result of, for example, altered expression of the marker, altered protein level of the marker, altered structure of the marker, or, e.g., an altered interaction with other proteins involved in the same or different pathway as the marker, or altered interaction with transcriptional activators or inhibitors.

The term “altered structure” of a marker refers to the presence of mutations or allelic variants within the marker gene or maker protein, e.g., mutations which affect expression or activity of the marker, as compared to the normal or wild-type gene or protein. For example, mutations include, but are not limited to substitutions, deletions, or addition mutations. Mutations may be present in the coding or non-coding region of the marker.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions may be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. “Inactivating antibodies” refers to antibodies that do not induce the complement system.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen. It has been shown that the antigen-binding function of an antibody may be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv may be linked to human immunoglobulin constant region cDNA or genomic sequences, in order to generate expression vectors encoding complete IgG polypeptides or other isotypes. VH and VL may also be used in the generation of Fab , Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S.M., et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S.M., et al. (1994) Mol. Immunol. 31:1047-1058). Antibody portions, such as Fab and F(ab′)2 fragments, may be prepared from whole antibodies using conventional techniques, such as papain or pepsin digestion, respectively, of whole antibodies. Moreover, antibodies, antibody portions and immunoadhesion polypeptides may be obtained using standard recombinant DNA techniques, as described herein.

Antibodies may be polyclonal or monoclonal; xenogeneic, allogeneic, or syngeneic; or modified forms thereof (e.g., humanized, chimeric, etc.). Antibodies may also be fully human. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper’s fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenstrom’s macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms’ tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin’s disease and non-Hodgkin’s disease), multiple myeloma, Waldenstrom’s macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The terms “CDR”, and its plural “CDRs”, refer to a complementarity determining region (CDR) of which three make up the binding character of a light chain variable region (CDR-L1, CDR-L2 and CDR-L3) and three make up the binding character of a heavy chain variable region (CDR-H1, CDR-H2 and CDR-H3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat, Chothia, and/or MacCallum et al., (Kabat et al., in “Sequences of Proteins of Immunological Interest,” 5^{th} Edition, U.S. Department of Health and Human Services, 1992; Chothia et al. (1987) J. Mol. Biol. 196, 901; and MacCallum et al., J. Mol. Biol. (1996) 262, 732, each of which is incorporated by reference in its entirety).

As used herein, the term “classifying” includes “to associate” or “to categorize” a sample with a disease state. In certain instances, “classifying” is based on statistical evidence, empirical evidence, or both. In certain embodiments, the methods and systems of classifying use of a so-called training set of samples having known disease states. Once established, the training data set serves as a basis, model, or template against which the features of an unknown sample are compared, in order to classify the unknown disease state of the sample. In certain instances, classifying the sample is akin to diagnosing the disease state of the sample. In certain other instances, classifying the sample is akin to differentiating the disease state of the sample from another disease state.

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

“Complement [to]” or “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. In another embodiment, all nucleotide residues of the first portion are capable of base pairing with nucleotide residues in the second portion.

As used herein, the term “composite antibody” refers to an antibody which has variable regions comprising germline or non-germline immunoglobulin sequences from two or more unrelated variable regions. Additionally, the term “composite, human antibody” refers to an antibody which has constant regions derived from human germline or non-germline immunoglobulin sequences and variable regions comprising human germline or non-germline sequences from two or more unrelated human variable regions. A composite, human antibody is useful as an effective component in a therapeutic agent according to the present invention since the antigenicity of the composite, human antibody in the human body is lowered.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (may be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels may be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient may be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

As used herein, the term “Fc region” is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the present invention include human IgG1, IgG2 (IgG2A, IgG2B), IgG3 and IgG4.

As used herein, “Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcyRI, FcyRII, and FcyRIII subclasses, including allelic variants and alternatively spliced forms of these receptors, FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

A molecule is “fixed” or “affixed” to a substrate if it is covalently or non-covalently associated with the substrate such the substrate may be rinsed with a fluid (e.g. standard saline citrate, pH 7.4) without a substantial fraction of the molecule dissociating from the substrate.

As used herein, “Framework” or “FR” residues are those variable-domain residues other than the HVR residues as herein defined.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

As used herein, the term “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the term “host cell” is intended to refer to a cell into which a nucleic acid of the present invention, such as a recombinant expression vector of the present invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “humanized antibody”, as used herein, is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. Humanized antibodies may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

As used herein, the term “hypervariable region,” “HVR,” or “HV,” refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. (2000) Immunity 13, 37-45; Johnson and Wu in Methods in Molecular Biology 248, 1-25 (Lo, ed., Human Press, Totowa, NJ, 2003)). Indeed, naturally occurring camelid antibodies consisting of a heavy chain only are functional and stable in the absence of light chain (see, e.g., Hamers-Casterman et al. (1993) Nature 363:446-448 (1993) and Sheriff et al. (1996) Nature Struct. Biol. 3, 733-736).

As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “immune disorder” includes immune diseases, conditions, and predispositions to, including, but not limited to, cancer, chronic inflammatory disease and disorders (including, e.g., Crohn’s disease, inflammatory bowel disease, reactive arthritis, and Lyme disease), insulin-dependent diabetes, organ specific autoimmunity (including, e.g., multiple sclerosis, Hashimoto’s thyroiditis, autoimmune uveitis, and Grave’s disease), contact dermatitis, psoriasis, graft rejection, graft versus host disease, sarcoidosis, atopic conditions (including, e.g., asthma and allergy including, but not limited to, allergic rhinitis and gastrointestinal allergies such as food allergies), eosinophilia, conjunctivitis, glomerular nephritis, systemic lupus erythematosus, scleroderma, certain pathogen susceptibilities such as helminthic (including, e.g., leishmaniasis) and certain viral infections (including, e.g., HIV and bacterial infections such as tuberculosis and lepromatous leprosy) and malaria.

As used herein, the term “immune response” includes T cell mediated and/or B cell mediated immune responses. Exemplary immune responses include T cell responses, e.g., cytokine production, and cellular cytotoxicity. In addition, the term immune response includes immune responses that are indirectly effected by T cell activation, e.g., antibody production (humoral responses) and activation of cytokine responsive cells, e.g., macrophages.

As used herein, the term “inhibiting” and grammatical equivalents thereof refer decrease, limiting, and/or blocking a particular action, function, or interaction. In one embodiment, the term refers to reducing the level of a given output or parameter to a quantity (e.g., background staining, PD-1 signaling, PD-1 immunoinhibitory function, and the like) which is at least 10%, 15%, 20%, 25%, 30%, 35%, 40%, 45%, 50%, 55%, 60%, 65%, 70%, 75%, 80%, 85%, 90%, 95%, 99% or less than the quantity in a corresponding control. A reduced level of a given output or parameter need not, although it may, mean an absolute absence of the output or parameter. The invention does not require, and is not limited to, methods that wholly eliminate the output or parameter. The given output or parameter may be determined using methods well known in the art, including, without limitation, immunohistochemical, molecular biological, cell biological, clinical, and biochemical assays, as discussed herein and in the examples. The opposite terms “promoting,” “increasing,” and grammatical equivalents thereof refer to the increase in the level of a given output or parameter that is the reverse of that described for inhibition or decrease.

As used herein, the term “interaction”, when referring to an interaction between two molecules, refers to the physical contact (e.g., binding) of the molecules with one another. Generally, such an interaction results in an activity (which produces a biological effect) of one or both of said molecules. The activity may be a direct activity of one or both of the molecules, (e.g., signal transduction). Alternatively, one or both molecules in the interaction may be prevented from binding their ligand, and thus be held inactive with respect to ligand binding activity (e.g., binding its ligand and triggering or inhibiting an immune response). To inhibit such an interaction results in the disruption of the activity of one or more molecules involved in the interaction. To enhance such an interaction is to prolong or increase the likelihood of said physical contact, and prolong or increase the likelihood of said activity.

As used herein, the term an “isolated antibody” is intended to refer to an antibody which is substantially free of other antibodies having different antigenic specificities (e.g., an isolated antibody that specifically binds to human PD-1 and is substantially free of antibodies that do not bind to the PD-1). An isolated antibody that specifically binds to human PD-1 may, however, have cross-reactivity to other PD-1 proteins, respectively, from different species. For example, in some embodiments, the antibody maintains specific binding affinity for at least two species, such as human and mouse, or other mammal or non-mammal species. However, in some embodiments, the antibody maintains higher or indeed specific affinity and selectivity for human PD-1. In addition, an isolated antibody is typically substantially free of other cellular material and/or chemicals. In one embodiment of the present invention, a combination of “isolated” monoclonal antibodies having different specificities to human PD-1 are combined in a well-defined composition.

As used herein, an “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a target polypeptide (e.g., immunoglobulin) or fragment thereof, in which the protein is separated from cellular components of the cells from which it is isolated or recombinantly produced. In one embodiment, the language “substantially free of cellular material” includes preparations of target protein or fragment thereof, having less than about 30% (by dry weight) of non-target protein (also referred to herein as a “contaminating protein”), more preferably less than about 20% of non-target protein, still more preferably less than about 10% of non-target protein, and most preferably less than about 5% non-target protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM or IgG1) that is encoded by heavy chain constant region genes.

As used herein, the term “K_{D}” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

As used herein, a “kit” is any manufacture (e.g. a package or container) comprising at least one reagent, e.g. a probe, for specifically detecting or modulating the expression of a marker of the present invention. The kit may be promoted, distributed, or sold as a unit for performing the methods of the present invention.

As used herein, the term “monoclonal antibody”, refers to an antibody which displays a single binding specificity and affinity for a particular epitope. Accordingly, the term “human monoclonal antibody” refers to an antibody which displays a single binding specificity and which has variable and constant regions derived from human germline or non-germline immunoglobulin sequences. In one embodiment, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

A “marker” is a gene whose altered level of expression in a tissue or cell from its expression level in normal or healthy tissue or cell is associated with a disease state, such as cancer. A “marker nucleic acid” is a nucleic acid (e.g., mRNA, cDNA) encoded by or corresponding to a marker of the present invention. Such marker nucleic acids include DNA (e.g., cDNA) comprising the entire or a partial sequence of any of the nucleic acid sequences set forth in the Sequence Listing or the complement of such a sequence. The marker nucleic acids also include RNA comprising the entire or a partial sequence of any of the nucleic acid sequences set forth in the Sequence Listing or the complement of such a sequence, wherein all thymidine residues are replaced with uridine residues. A “marker protein” is a protein encoded by or corresponding to a marker of the present invention. A marker protein comprises the entire or a partial sequence of any of the sequences set forth in the Sequence Listing. In some embodiments, PD-1 is used as a marker. The terms “protein” and “polypeptide” are used interchangeably.

As used herein, the term “modulate” includes up-regulation and down-regulation, e.g., enhancing or inhibiting a response.

The “normal” level of expression of a marker is the level of expression of the marker in cells of a subject, e.g., a human patient, not afflicted with a disease or disorder related to aberrant marker levels. An “over-expression” or “significantly higher level of expression” of a marker refers to an expression level in a test sample that is greater than the standard error of the assay employed to assess expression, and is preferably at least twice, and more preferably three, four, five or ten times the expression level of the marker in a control sample (e.g., sample from a healthy subjects not having the marker associated disease) and preferably, the average expression level of the marker in several control samples. A “significantly lower level of expression” of a marker refers to an expression level in a test sample that is at least twice, and more preferably three, four, five or ten times lower than the expression level of the marker in a control sample (e.g., sample from a healthy subject not having the marker associated disease) and preferably, the average expression level of the marker in several control samples.

As used herein, the term “nucleic acid molecule” is intended to include DNA molecules and RNA molecules. A nucleic acid molecule may be single-stranded or double-stranded, but preferably is double-stranded DNA. As used herein, the term “isolated nucleic acid molecule” in reference to nucleic acids encoding antibodies or antibody portions (e.g., V_{H}, V_{L}, CDR3) that bind to PD-1, is intended to refer to a nucleic acid molecule in which the nucleotide sequences encoding the antibody or antibody portion are free of other nucleotide sequences encoding antibodies or antibody portions that bind antigens other than PD-1, which other sequences may naturally flank the nucleic acid in human genomic DNA.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

An “over-expression” or “significantly higher level of expression” of a marker refers to an expression level in a test sample that is greater than the standard error of the assay employed to assess expression, and is preferably at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the marker in a control sample (e.g., sample from a healthy subject not having the marker associated disease) and preferably, the average expression level of the marker in several control samples. A “significantly lower level of expression” of a marker refers to an expression level in a test sample that is at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the marker in a control sample (e.g., sample from a healthy subject not having the marker associated disease) and preferably, the average expression level of the marker in several control samples.

The samples may be collected from individuals repeatedly over a longitudinal period of time (e.g., once or more on the order of days, weeks, months, annually, biannually, etc.). Obtaining numerous samples from an individual over a period of time may be used to verify results from earlier detections and/or to identify an alteration in biological pattern as a result of, for example, disease progression, drug treatment, etc. For example, subject samples may be taken and monitored every month, every two months, or combinations of one, two, or three month intervals according to the present invention. In addition, the biomarker amount and/or activity measurements of the subject obtained over time may be conveniently compared with each other, as well as with those of normal controls during the monitoring period, thereby providing the subject’s own values, as an internal, or personal, control for long-term monitoring.

Samples may contain live cells/tissue, fresh frozen cells, fresh tissue, biopsies, fixed cells/tissue, cells/tissue embedded in a medium, such as paraffin, histological slides, or any combination thereof.

Sample preparation and separation may involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation may also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample may be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus, internally, or at the carboxyl-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. They may be, for example, at least and/or including 10, 15, 20, 25, 30, 35, 40, 45, 50, 55, 60, 65, 70, 75, 80, 85, 90, 95, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340 or more long so long as they are less than the length of the full-length polypeptide. Alternatively, they may be no longer than and/or excluding such a range so long as they are less than the length of the full-length polypeptide.

The term “probe” refers to any molecule which is capable of selectively binding to a specifically intended target molecule, for example, a nucleotide transcript or protein encoded by or corresponding to a marker. Probes may be either synthesized by one skilled in the art, or derived from appropriate biological preparations. For purposes of detection of the target molecule, probes may be specifically designed to be labeled, as described herein. Examples of molecules that may be utilized as probes include, but are not limited to, RNA, DNA, proteins, antibodies, and organic molecules.

As used herein, the term “rearranged” refers to a configuration of a heavy chain or light chain immunoglobulin locus wherein a V segment is positioned immediately adjacent to a D-J or J segment in a conformation encoding essentially a complete V_{H} and V_{L} domain, respectively. A rearranged immunoglobulin gene locus may be identified by comparison to germline DNA; a rearranged locus will have at least one recombined heptamer/nonamer homology element.

As used herein, the term “recombinant host cell” (or simply “host cell”), is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “recombinant human antibody” includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline and/or non-germline immunoglobulin sequences. In certain embodiments, however, such recombinant human antibodies may be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The present invention “response” is generally related to for example, determining the effects on progression, efficacy, or outcome of a clinical intervention. In some embodiments, responses relate directly to a change in tumor mass and/or volume after initiation of clinical intervention. For example, hyperproliferative disorder responses may be assessed according to the size of a tumor after systemic intervention compared to the initial size and dimensions as measured by CT, PET, mammogram, ultrasound or palpation. Response may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment may be done early after the onset of the clinical intervention, e.g., after a few hours, days, weeks or preferably after a few months. A typical endpoint for response assessment is upon termination of the clinical intervention or upon surgical removal of residual tumor cells and/or the tumor bed.

As used herein, the term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using human PD-1 as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.”

As used herein, “subject” refers to any healthy animal, mammal or human, or any animal, mammal or human afflicted with a disease or disorder related to aberrant marker levels. The term “subject” is interchangeable with “patient”. The term “non-human animal” includes all vertebrates, e.g., mammals and non-mammals, such as non-human primates, sheep, dog, cow, chickens, amphibians, reptiles, etc.

The language “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30% (by dry weight) of chemical precursors or non-antibody, polypeptide, peptide or fusion protein chemicals, more preferably less than about 20% chemical precursors or non-antibody, polypeptide, peptide or fusion protein chemicals, still more preferably less than about 10% chemical precursors or non-antibody, polypeptide, peptide or fusion protein chemicals, and most preferably less than about 5% chemical precursors or non- antibody, polypeptide, peptide or fusion protein chemicals.

As used herein, the term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment may be expanded to include response to chemotherapy, probability of survival, probability of metastasis within a given time period, and probability of tumor recurrence.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a marker of the present invention and normal post-transcriptional processing (e.g. splicing), if any, of the RNA transcript, and reverse transcription of the RNA transcript.

As used herein, the term “T cell” includes CD4+ T cells and CD8+ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells) as well as other antigen presenting cells (e.g., keratinocytes, endothelial cells, astrocytes, fibroblasts, oligodendrocytes).

As used herein, the term “unrearranged” or “germline configuration” in reference to a V segment refers to the configuration wherein the V segment is not recombined so as to be immediately adjacent to a D or J segment.

As used herein, the term “vector” refers to a nucleic acid capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 81%, 82%, 83%, 84%, 85%, 86%, 87%, 88%, 89%, 90%, 91%, 92%, 93%, 94%, 95%, 96%, or more of the nucleotides, and more preferably at least about 97%, 98%, 99% or more of the nucleotides. Alternatively, substantial homology exists when the segments will hybridize under selective hybridization conditions, to the complement of the strand.

The percent identity between two sequences is a function of the number of identical positions shared by the sequences (i.e., % identity= # of identical positions/total # of positions × 100), taking into account the number of gaps, and the length of each gap, which need to be introduced for optimal alignment of the two sequences. The comparison of sequences and determination of percent identity between two sequences may be accomplished using a mathematical algorithm, as described in the non-limiting examples below.

The percent identity between two nucleotide sequences may be determined using the GAP program in the GCG software package (available on the world wide web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences may also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (J. Mol. Biol. (48):444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention may further be used as a “query sequence” to perform a search against public databases to, for example, identify related sequences. Such searches may be performed using the NBLAST and XBLAST programs (version 2.0) of Altschul, et al. (1990) J. Mol. Biol. 215:403 10. BLAST nucleotide searches may be performed with the NBLAST program, score=100, wordlength=12 to obtain nucleotide sequences homologous to the nucleic acid molecules of the present invention. BLAST protein searches may be performed with the XBLAST program, score=50, wordlength=3 to obtain amino acid sequences homologous to the protein molecules of the present invention. To obtain gapped alignments for comparison purposes, Gapped BLAST may be utilized as described in Altschul et al., (1997) Nucleic Acids Res. 25(17):3389 3402. When utilizing BLAST and Gapped BLAST programs, the default parameters of the respective programs (e.g., XBLAST and NBLAST) may be used (available on the world wide web at the NCBI website).

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (see, F. Ausubel, et al., ed. Current Protocols in Molecular Biology, Greene Publishing and Wiley Interscience, New York (1987)).

### II. Monoclonal Antibodies, Immunoglobulins, and Polypeptides

The present invention relates, in part, to isolated bispecific molecules, which may include monoclonal antibodies or fragments thereof that are directed against PD-1 and against an antigen on the opposing surface of an adjacent cell.

The term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

The nucleic acid and amino acid sequences of a representative human PD-1 biomarker is available to the public at the GenBank database under NM_005018.2 and NP_005009.2 and is shown in Table 2 (see also Ishida et al. (1992) 20 EMBO J 11:3887; Shinohara et al. (1994) Genomics 23:704; U.S. Pat. 5,698,520). PD-1 has an extracellular region containing immunoglobulin superfamily domain, a transmembrane domain, and an intracellular region including an immunoreceptor tyrosine-based inhibitory motif (ITIM) (Ishida et al. (1992) EMBO J. 11:3887; Shinohara et al. (1994) Genomics 23:704; and U.S. Pat. 5,698,520) and an immunoreceptor tyrosine-based switch motif (ITSM). These features also define a larger family of polypeptides, called the immunoinhibitory receptors, which also includes gp49B, PIR-B, and the killer inhibitory receptors (KIRs) (Vivier and Daeron (1997) Immunol. Today 18:286). It is often assumed that the tyrosyl phosphorylated ITIM and ITSM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al. (1999) Immunol. Today 20(6):285-8). Nucleic acid and polypeptide sequences of PD-1 orthologs in organisms other than humans are well known and include, for example, mouse PD-1 (NM_008798.2 and NP_032824.1), rat PD-1 (NM_001106927.1 and NP_001100397.1), dog PD-1 (XM_543338.3 and XP_543338.3), cow PD-1 (NM_001083506.1 and NP_001076975.1), and chicken PD-1 (XM_422723.3 and XP_422723.2).

PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells.

The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell costimulatory or inhibitory signals, and the ability to modulate the immune response.

In some embodiments, a condition such as cancer is responsive to PD-1 blockade alone. In other embodiments, a condition such as cancer is responsive to PD-1 blockade alone, but is significantly or synergistically more responsive when treated with PD-1 blockade and another therapy in combination. Many conditions responsive to PD-1 blockade alone are known and include, without limitation, melanoma (e.g., advanced or metastatic melanoma), lung cancer (e.g., non-small cell lung cancer and small cell lung cancer), breast cancer (e.g., HER-2 negative breast cancer, estrogen-receptor+/HER-2-breast cancer, and triple negative breast cancer), pancreatic cancer (e.g., pancreatic adenocarcinoma), and Hodgkin lymphoma, as well as bladder, gastric, head and neck, renal, prostate, gynecologic, and hematologic cancers.

The term “PD-1 ligand” refers to binding partners of the PD-1 receptor and includes both PD-L1 (Freeman et al. (2000) J. Exp. Med. 192:1027) and PD-L2 (Latchman et al. (2001) Nat. Immunol. 2:261). At least two types of human PD-1 ligand polypeptides exist. PD-1 ligand proteins comprise a signal sequence, and an IgV domain, an IgC domain, a transmembrane domain, and a short cytoplasmic tail. Both PD-L1 (See Freeman et al. (2000) J. Exp. Med. 192:1027 for sequence data) and PD-L2 (See Latchman et al. (2001) Nat. Immunol. 2:261 for sequence data) are members of the B7 family of polypeptides. Both PD-L1 and PD-L2 are expressed in placenta, spleen, lymph nodes, thymus, and heart. Only PD-L2 is expressed in pancreas, lung and liver, while only PD-L1 is expressed in fetal liver. Both PD-1 ligands are upregulated on activated monocytes and dendritic cells, although PD-L1 expression is broader. For example, PD-L1 is known to be constitutively expressed and upregulated to higher levels on murine hematopoietic cells (e.g., T cells, B cells, macrophages, dendritic cells (DCs), and bone marrow-derived mast cells) and non-hematopoietic cells (e.g., endothelial, epithelial, and muscle cells), whereas PD-L2 is inducibly expressed on DCs, macrophages, and bone marrow-derived mast cells (see Butte et al. (2007) Immunity 27:111).

PD-1 ligands comprise a family of polypeptides having certain conserved structural and functional features. The term “family” when used to refer to proteins or nucleic acid molecules, is intended to mean two or more proteins or nucleic acid molecules having a common structural domain or motif and having sufficient amino acid or nucleotide sequence homology, as defined herein. Such family members may be naturally or non-naturally occurring and may be from either the same or different species. For example, a family may contain a first protein of human origin, as well as other, distinct proteins of human origin or alternatively, may contain homologues of non-human origin. Members of a family may also have common functional characteristics. PD-1 ligands are members of the B7 family of polypeptides. The term “B7 family” or “B7 polypeptides” as used herein includes costimulatory polypeptides that share sequence homology with B7 polypeptides, e.g., with B7-1, B7-2, B7h (Swallow et al. (1999) Immunity 11:423), and/or PD-1 ligands (e.g., PD-L1 or PD-L2). For example, human B7-1 and B7-2 share approximately 26% amino acid sequence identity when compared using the BLAST program at NCBI with the default parameters (Blosum62 matrix with gap penalties set at existence 11 and extension 1 (See the NCBI website). The term B7 family also includes variants of these polypeptides which are capable of modulating immune cell function. The B7 family of molecules share a number of conserved regions, including signal domains, IgV domains and the IgC domains. IgV domains and the IgC domains are art-recognized Ig superfamily member domains. These domains correspond to structural units that have distinct folding patterns called Ig folds. Ig folds are comprised of a sandwich of two β sheets, each consisting of anti-parallel β strands of 5-10 amino acids with a conserved disulfide bond between the two sheets in most, but not all, IgC domains of Ig, TCR, and MHC molecules share the same types of sequence patterns and are called the C1-set within the Ig superfamily. Other IgC domains fall within other sets. IgV domains also share sequence patterns and are called V set domains. IgV domains are longer than IgC domains and contain an additional pair of β strands.

Preferred B7 polypeptides are capable of providing costimulatory or inhibitory signals to immune cells to thereby promote or inhibit immune cell responses. For example, B7 family members that bind to costimulatory receptors increase T cell activation and proliferation, while B7 family members that bind to inhibitory receptors reduce costimulation. Moreover, the same B7 family member may increase or decrease T cell costimulation. For example, when bound to a costimulatory receptor, PD-1 ligand may induce costimulation of immune cells or may inhibit immune cell costimulation, e.g., when present in soluble form. When bound to an inhibitory receptor, PD-1 ligand polypeptides may transmit an inhibitory signal to an immune cell. Preferred B7 family members include B7-1, B7-2, B7h, PD-L1 or PD-L2 and soluble fragments or derivatives thereof. In one embodiment, B7 family members bind to one or more receptors on an immune cell, e.g., CTLA4, CD28, ICOS, PD-1 and/or other receptors, and, depending on the receptor, have the ability to transmit an inhibitory signal or a costimulatory signal to an immune cell, preferably a T cell.

Modulation of a costimulatory signal results in modulation of effector function of an immune cell. Thus, the term “PD-1 ligand activity” includes the ability of a PD-1 ligand polypeptide to bind its natural receptor(s) (e.g. PD-1 or B7-1), the ability to modulate immune cell costimulatory or inhibitory signals, and the ability to modulate the immune response.

In various embodiments, the antibodies or their variants (or nucleic acids encoding them) may comprise a sequence provided in Example 2 or Example 4. A few of those sequences are also provided below in Table 1.

TABLE 1

Exemplary Variable Domain Sequences
SEQ ID NO: 21 (VH of anti-H2Kb)
DVQLVESGGGLVQPGGSRKLSCAASGFTFSSFGMHWVRQAPEKGLEWVAYISSGSNTIYYADTVKGR FTISRDNPKNTLFLQMTSLRSEDTAMYYCALTTGSWFAYWGQGTLVTVSA

SEQ ID NO: 22 (VL of anti-H2Kb)
DILLTQSPAILSVRPGERVSFSCRASQSLGTSIHWYQQRTDGSPRLLIKYASESISGIPSRFSGSGS GTDFTLSINSVESEDIADYYCQQSNSWPITFGAGTKLELK

SEQ ID NO: 30 (VH of anti-PD-1)
QVQLQQSGAELVKPGSSVKISCKASGYTFTSHFIHWIKQQPGNGLEWIGGIYPGDGDTEYNQQFNGK ATLTADKSSSTAYMRLSSLTSEDSAVYFCATRVPSYWFFDFWGPGTMVTVSS

SEQ ID NO: 31 (VL of anti-PD-1)
DVALTQTPVAQPVTLGDQASISCRSSQSLVHSNGRTYLEWYLQKPGQSPQLLIYKVSNRFSGVPDRF IGSGSGSDFTLTISRVEPEDLGVYYCFQATHDPNTFGAGTKLELK
* Included in Table 1 are RNA nucleic acid molecules (e.g., thymines replaced with uridines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO or biomarker described in Table 1, or a portion thereof. Such nucleic acid molecules may have a function of the full-length nucleic acid as described further herein.
* Included in Table 1 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO or biomarker described in Table 1, or a portion thereof. Such polypeptides may have a function of the full-length polypeptide as described further herein.

Since it is well known in the art that antibody heavy and light chain CDR3 domains play a particularly important role in the binding specificity/affinity of an antibody for an antigen, the recombinant monoclonal antibodies of the present invention prepared as set forth above preferably comprise the heavy and light chain CDR3s of variable regions of the present invention. The antibodies further may comprise the CDR2s of variable regions of the present invention. The antibodies further may comprise the CDR1s of variable regions of the present invention. In other embodiments, the antibodies may comprise any combinations of the CDRs. These CDRs, for a given definition (e.g., Kabat), may be determined from the provided VH/VL sequences.

The CDR1, 2, and/or 3 regions of the engineered antibodies described above may comprise the exact amino acid sequence(s) as those of variable regions of the present invention disclosed herein. However, the ordinarily skilled artisan will appreciate that some deviation from the exact CDR sequences may be possible while still retaining the ability of the antibody to bind PD-1 effectively (e.g., conservative sequence modifications). Accordingly, in another embodiment, the engineered antibody may be composed of one or more CDRs that are, for example, 50%, 60%, 70%, 80%, 85%, 90%, 91%, 92%, 93%, 94%, 95%, 96%, 97%, 98%, 99%, or 99.5% identical to one or more CDRs of the present invention.

The heavy chain variable domain of the monoclonal antibodies of the present invention may comprise or consist of the vH amino acid sequence set forth in Table 1 and/or the light chain variable domain of the monoclonal antibodies of the present invention may comprise or consist of the vL amino acid sequence set forth in Table 1.

The monoclonal antibodies of the present invention may be produced and modified by any technique well known in the art. Similarly, such monoclonal antibodies may be chimeric, preferably chimeric mouse/human antibodies. In some embodiments, the monoclonal antibodies are humanized antibodies such that the variable domain comprises human acceptor frameworks regions, and optionally human constant domain where present, and non-human donor CDRs, such as mouse CDRs as defined above.

The present invention further provides fragments of said monoclonal antibodies which include, but are not limited to, Fv, Fab, F(ab′)2, Fab′, dsFv, scFv, sc(Fv)2 and diabodies; and multispecific antibodies formed from antibody fragments. For example, a number of immunoinhibitory molecules, such as PD-1, PD-L2, PD-L1, CTLA-4, and the like, may be detected in a bispecific or multispecific manner in order to efficiently characterize the expression of such molecules.

Other fragments of the monoclonal antibodies of the present invention are also contemplated. For example, individual immunoglobulin heavy and/or light chains are provided, wherein the variable domains thereof comprise at least a CDR present in the sequences listed in Table 1. In one embodiment, the immunoglobulin heavy chain comprises at least a CDR having a sequence that is at least 80%, 85%, 90%, 91%, 92%, 93%, 94%, 95%, 96%, 97%, 98%, 99%, 99.5% or 100% identical from the group of heavy chain or light chain variable domain CDRs presented in the sequences listed in Table 1. In another embodiment, an immunoglobulin light chain comprises at least a CDR having a sequence that is at least 80%, 85%, 90%, 91%, 92%, 93%, 94%, 95%, 96%, 97%, 98%, 99%, 99.5% or 100% identical from the group of light chain or heavy chain variable domain CDRs described herein.

In some embodiments, the immunoglobulin heavy and/or light chain comprises a variable domain comprising at least one of CDR-L1, CDR-L2, CDR-L3, CDR-H1, CDR-H2, or CDR-H3 described herein. Such immunoglobulin heavy chains may comprise or consist of at least one of CDR-H1, CDR-H2, and CDR-H3. Such immunoglobulin light chains may comprise or consist of at least one of CDR-L1, CDR-L2, and CDR-L3.

In other embodiments, an immunoglobulin heavy and/or light chain according to the present invention comprises or consists of a vH or vL variable domain sequence, respectively, provided in Table 1.

The present invention further provides polypeptides which have a sequence selected from the group consisting of vH variable domain, vL variable domain, CDR-L1, CDR-L2, CDR-L3, CDR-H1, CDR-H2, and CDR-H3 sequences described herein.

Antibodies, immunoglobulins, and polypeptides of the invention may be use in an isolated (e.g., purified) form or contained in a vector, such as a membrane or lipid vesicle (e.g. a liposome).

Some exemplary PD-1 sequences are provided below in Table 2.

TABLE 2

Representative PD-1 Sequences
Human PD-1 cDNA Acid Sequence (NM_005018.2, CDS from position 69 to position 935)

| | | | | | | |
|---|---|---|---|---|---|---|
| 1 | agtttccctt | ccgctcacct | ccgcctgagc | agtggagaag | gcggcactct | ggtggggctg |
| 61 | ctccaggcat | gcagatccca | caggcgccct | ggccagtcgt | ctgggcggtg | ctacaactgg |
| 121 | gctggcggcc | aggatggttc | ttagactccc | cagacaggcc | ctggaacccc | cccaccttct |
| 181 | ccccagccct | gctcgtggtg | accgaagggg | acaacgccac | cttcacctqc | agcttctcca |
| 241 | acacatcgga | gagcttcgtg | ctaaactggt | accgcatgag | ccccagcaac | cagacggaca |
| 301 | agctggccgc | cttccccgag | gaccgcagcc | agcccggcca | ggactgccgc | ttccgtgtca |
| 361 | cacaactgcc | caacgggcgt | gacttccaca | tgagcgtggt | cagggcccgg | cgcaatgaca |
| 421 | gcggcaccta | cctctgtggg | gccatctccc | tggcccccaa | ggcgcagatc | aaagagagcc |
| 481 | tgcgggcaga | gctcagggtg | acagagagaa | gggcagaagt | gcccacagcc | caccccagcc |
| 541 | cctcacccag | gccagccggc | cagttccaaa | ccctggtggt | tggtgtcgtg | ggcggcctgc |
| 601 | tgggcagcct | ggtgctgcta | gtctgggtcc | tggccgtcat | ctgctcccgg | gccgcacgag |
| 661 | ggacaatagg | agccaggcgc | accggccagc | ccctgaagga | ggacccctca | gccgtgcctg |
| 721 | tgttctctgt | ggactatggg | gagctggatt | tccagtggcg | agagaagacc | ccggagcccc |
| 781 | ccgtgccctg | tgtccctgag | cagacggagt | atgccaccat | tgtctttcct | agcggaatgg |
| 841 | gcacctcatc | ccccgcccgc | aggggctcag | ctgacggccc | tcggagtgcc | cagccactga |
| 901 | ggcctgagga | tggacactgc | tcttggcccc | tctgaccggc | ttccttggcc | accagtgttc |
| 961 | tgcagaccct | ccaccatgag | cccgggtcag | cgcatttcct | caggagaagc | aggcagggtg |
| 1021 | caggccattg | caggccgtcc | aggggctgag | ctgcctgggg | gcgaccgggg | ctccagcctg |
| 1081 | cacctgcacc | aggcacagcc | ccaccacagg | actcatgtct | caatgcccac | agtgagccca |
| 1141 | ggcagcaggt | gtcaccgtcc | cctacaggga | gggccagatg | cagtcactgc | ttcaggtcct |
| 1201 | gccagcacag | agctgcctgc | gtccagctcc | ctgaatctct | gctgctgctg | ctgctgctgc |
| 1261 | tgctgctgcc | tgcggcccgg | ggctgaaggc | gccgtggccc | tgcctgacgc | cccggagcct |
| 1321 | cctgcctgaa | cttgggggct | ggttggagat | ggccttggag | cagccaaggt | gcccctggca |
| 1381 | gtggcatccc | gaaacgccct | ggacgcaggg | cccaagactg | ggcacaggag | tgggaggtac |
| 1441 | atggggctgg | ggactcccca | ggagttatct | gctccctgca | ggcctagaga | agtttcaggg |
| 1501 | aaggtcagaa | gagctcctgg | ctgtggtggg | cagggcagga | aacccctcca | cctttacaca |
| 1561 | tgcccaggca | gcacctcagg | ccctttgtgg | ggcagggaag | ctgaggcagt | aagcgggcag |
| 1621 | gcagagctgg | aggcctttca | ggcccagcca | gcactctggc | ctcctgccgc | cgcattccac |
| 1681 | cccagcccct | cacaccactc | gggagaggga | catcctacgg | tcccaaggtc | aggagggcag |
| 1741 | ggctggggtt | gactcaggcc | cctcccagct | gtggccacct | gggtgttggg | agggcagaag |
| 1801 | tgcaggcacc | tagggccccc | catgtgccca | ccctgggagc | tctccttgga | acccattcct |
| 1861 | gaaattattt | aaaggggttg | gccgggctcc | caccagggcc | tgggtgggaa | ggtacaggcg |
| 1921 | ttcccccggg | gcctagtacc | cccgccgtgg | cctatccact | cctcacatcc | acacactgca |
| 1981 | cccccactcc | tggggcaggg | ccaccagcat | ccaggcggcc | agcaggcacc | tgagtggctg |
| 2041 | ggacaaggga | tcccccttcc | ctgtggttct | attatattat | aattataatt | aaatatgaga |
| 2101 | gcatgctaag | gaaaa (SEQ | ID NO: 27) | | | |
| 1 | mqipqapwpv | vwavlqlgwr | pgwfldspdr | pwnpptfspa | llvvtegdna | tftcsfsnts |
| 61 | esfvlnwyrm | spsnqtdkla | afpedrsqpg | qdcrfrvtql | pngrdfhmsv | vrarrndsgt |
| 121 | ylcgaislap | kaqikeslra | elrvterrae | vptahpspsp | rpagqfqtlv | vgvvggllgs |
| 181 | lvllvwvlav | icsraargti | garrtgqplk | edpsavpvfs | vdygeldfqw | rektpeppvp |
| 241 28) | cvpeqteyat | ivfpsgmgts | sparrgsadg | prsaqplrpe | dghcswpl | (SEQ ID NO: |
| 1 | tgagcagcgg | ggaggaggaa | gaggagactg | ctactgaagg | cgacactgcc | aggggctctg |
| 61 | ggcatgtggg | tccggcaggt | accctggtca | ttcacttggg | ctgtgctgca | gttgagctgg |
| 121 | caatcagggt | ggcttctaga | ggtccccaat | gggccctgga | ggtccctcac | cttctaccca |
| 181 | gcctggctca | cagtgtcaga | gggagcaaat | gccaccttca | cctgcagctt | gtccaactgg |
| 241 | tcggaggatc | ttatgctgaa | ctggaaccgc | ctgagtccca | gcaaccagac | tgaaaaacag |
| 301 | gccgccttct | gtaatggttt | gagccaaccc | gtccaggatg | cccgcttcca | gatcatacag |
| 361 | ctgcccaaca | ggcatgactt | ccacatgaac | atccttgaca | cacggcgcaa | tgacagtggc |
| 421 | atctacctct | gtggggccat | ctccctgcac | cccaaggcaa | aaatcgagga | gagccctgga |
| 481 | gcagagctcg | tggtaacaga | gagaatcctg | gagacctcaa | caagatatcc | cagcccctcg |
| 541 | cccaaaccag | aaggccggtt | tcaaggcatg | gtcattggta | tcatgagtgc | cctagtgggt |
| 601 | atccctgtat | tgctgctgct | ggcctgggcc | ctagctgtct | tctgctcaac | aagtatgtca |
| 661 | gaggccagag | gagctggaag | caaggacgac | actctgaagg | aggagccttc | agcagcacct |
| 721 | gtccctagtg | tggcctatga | ggagctggac | ttccagggac | gagagaagac | accagagctc |
| 781 | cctaccgcct | gtgtgcacac | agaatatgcc | accattgtct | tcactgaagg | gctgggtgcc |
| 841 | tcggccatgg | gacgtagggg | ctcagctgat | ggcctgcagg | gtcctcggcc | tccaagacat |
| 901 | gaggatggac | attgttcttg | gcctctttga | ccagattctt | cagccattag | catgctgcag |
| 961 | accctccaca | gagagcaccg | gtccgtccct | cagtcaagag | gagcatgcag | gctacagttc |
| 1021 | agccaaggct | cccagggtct | gagctagctg | gagtgacagc | ccagcgcctg | caccaattcc |
| 1081 | agcacatgca | ctgttgagtg | agagctcact | tcaggtttac | cacaagctgg | gagcagcagg |
| 1141 | cttcccggtt | tcctattgtc | acaaggtgca | gagctggggc | ctaagcctat | gtctcctgaa |
| 1201 | tcctactgtt | gggcacttct | agggacttga | gacactatag | ccaatggcct | ctgtgggttc |
| 1261 | tgtgcctgga | aatggagaga | tctgagtaca | gcctgctttg | aatggccctg | tgaggcaacc |
| 1321 | ccaaagcaag | ggggtccagg | tatactatgg | gcccagcacc | taaagccacc | cttgggagat |
| 1381 | gatactcagg | tgggaaattc | gtagactggg | ggactgaacc | aatcccaaga | tctggaaaag |
| 1441 | ttttgatgaa | gacttgaaaa | gctcctagct | tcgggggtct | gggaagcatg | agcacttacc |
| 1501 | aggcaaaagc | tccgtgagcg | tatctgctgt | ccttctgcat | gcccaggtac | ctcagttttt |
| 1561 | ttcaacagca | aggaaactag | ggcaataaag | ggaaccagca | gagctagagc | cacccacaca |
| 1621 | tccagggggc | acttgactct | ccctactcct | cctaggaacc | aaaaggacaa | agtccatgtt |
| 1681 | gacagcaggg | aaggaaaggg | ggatataacc | ttgacgcaaa | ccaacactgg | ggtgttagaa |
| 1741 | tctcctcatt | cactctgtcc | tggagttggg | ttctggctct | ccttcacacc | taggactctg |
| 1801 | aaatgagcaa | gcacttcaga | cagtcagggt | agcaagagtc | tagctgtctg | gtgggcaccc |
| 1861 | aaaatgacca | gggcttaagt | ccctttcctt | tggtttaagc | ccgttataat | taaatggtac |
| 1921 NO: 29) | caaaagcttt | aaaaaaaaaa | aaaaaaaaaa | aaaaaaaaaa | aaaaaaaaaa | aa (SEQ ID |
| 1 | mwvrqvpwsf | twavlqlswq | sgwllevpng | pwrsltfypa | wltvsegana | tftcslsnws |
| 61 | edlmlnwnrl | spsnqtekqa | afcnglsqpv | qdarfqiiql | pnrhdfhmni | ldtrrndsgi |
| 121 | ylcgaislhp | kakieespga | elvvterile | tstrypspsp | kpegrfqgmv | igimsalvgi |
| 181 | pvllllawal | avfcstsmse | argagskddt | lkeepsaapv | psvayeeldf | qgrektpelp |
| 241 30) | tacvhteyat | ivfteglgas | amgrrgsadg | lqgprpprhe | dghcswpl | (SEQ ID NO: |

* Included in Table 2 are RNA nucleic acid molecules (e.g., thymines replaced with uridines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO or biomarker described in Table 2, or a portion thereof. Such nucleic acid molecules may have a function of the full-length nucleic acid as described further herein.
* Included in Table 2 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO or biomarker described in Table 2, or a portion thereof. Such polypeptides may have a function of the full-length polypeptide as described further herein.

### III. Nucleic Acids, Vectors, and Recombinant Host Cells

A further object of the invention relates to nucleic acid sequences encoding bispecific molecules, monoclonal antibodies and fragments thereof, immunoglobulins, and polypeptides of the present invention.

In a particular embodiment, the invention relates to a nucleic acid sequence encoding the vH domain and/or vL domain of a mAb described herein, such as those disclosed in Table 1, Example 2, or Example 4.

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) may be introduced into a host cell, so as to transform the host and promote expression (e.g. transcription and translation) of the introduced sequence. Thus, a further object of the invention relates to a vector comprising a nucleic acid of the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell may be used. Examples of suitable vectors include pAGE107 (Miyaji H et al. 1990), pAGE103 (Mizukami T et al. 1987), pHSG274 (Brady G et al. 1984), pKCR (O′Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses may be found for instance in WO 95/14785, WO 96/22378, U.S. Pat. No. 5,882,877, U.S. Pat. No. 6,013,516, U.S. Pat. No. 4,861,719, U.S. Pat. No. 5,278,056 and WO 94/19478.

A further object of the present invention relates to a cell which has been transfected, infected or transformed by a nucleic acid and/or a vector according to the invention. The term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids of the present invention may be used to produce a recombinant polypeptide of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present invention also relates to a method of producing a recombinant host cell expressing an antibody or a polypeptide of the invention according to the invention, said method comprising the steps consisting of (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody or polypeptide. Such recombinant host cells may be used for the production of antibodies and polypeptides of the invention.

In another aspect, the present invention provides isolated nucleic acids that hybridize under selective hybridization conditions to a polynucleotide disclosed herein. Thus, the polynucleotides of this embodiment may be used for isolating, detecting, and/or quantifying nucleic acids comprising such polynucleotides. For example, polynucleotides of the present invention may be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. Preferably, the cDNA library comprises at least 80% full-length sequences, preferably, at least 85% or 90% full-length sequences, and, more preferably, at least 95% full-length sequences. The cDNA libraries may be normalized to increase the representation of rare sequences. Low or moderate stringency hybridization conditions are typically, but not exclusively, employed with sequences having a reduced sequence identity relative to complementary sequences. Moderate and high stringency conditions may optionally be employed for sequences of greater identity. Low stringency conditions allow selective hybridization of sequences having about 70% sequence identity and may be employed to identify orthologous or paralogous sequences. Optionally, polynucleotides of this invention will encode at least a portion of an antibody encoded by the polynucleotides described herein. The polynucleotides of this invention embrace nucleic acid sequences that may be employed for selective hybridization to a polynucleotide encoding an antibody of the present invention. See, e.g., Ausubel, supra; Colligan, supra, each entirely incorporated herein by reference.

### IV. Methods of Producing Bispecific Molecules and Antibodies

Bispecific molecules, antibodies and fragments thereof, immunoglobulins, and polypeptides of the present invention may be produced by any technique known in the art, such as, without limitation, any chemical, biological, genetic or enzymatic technique, either alone or in combination.

Knowing the amino acid sequence of the desired sequence, one skilled in the art may readily produce said antibodies or polypeptides, by standard techniques for production of polypeptides. For instance, they may be synthesized using well-known solid phase method, preferably using a commercially available peptide synthesis apparatus (such as that made by Applied Biosystems, Foster City, Calif.) and following the manufacturer’s instructions. Alternatively, antibodies and other polypeptides of the present invention may be synthesized by recombinant DNA techniques as is well-known in the art. For example, these fragments may be obtained as DNA expression products after incorporation of DNA sequences encoding the desired (poly)peptide into expression vectors and introduction of such vectors into suitable eukaryotic or prokaryotic hosts that will express the desired polypeptide, from which they may be later isolated using well-known techniques.

In particular, the present invention further relates to a method of producing an antibody or a polypeptide of the invention, which method comprises the steps consisting of: (i) culturing a transformed host cell according to the invention under conditions suitable to allow expression of said antibody or polypeptide; and (ii) recovering the expressed antibody or polypeptide.

Antibodies and other polypeptides of the present invention are suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) may also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Chimeric antibodies (e.g., mouse-human chimeras) of the present invention may be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody may be any region which belongs to human immunoglobulin, such as the IgG class or a subclass thereof, such as IgG1, IgG2, IgG3 and IgG4. Similarly, the CL of a human chimeric antibody may be any region which belongs to Ig, such as the kappa class or lambda class. chimeric and humanized monoclonal antibodies, comprising both human and non-human portions, which may be made using standard recombinant DNA techniques, are within the scope of the invention. Such chimeric and humanized monoclonal antibodies may be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies may be made according to standard protocols such as those disclosed in U.S. Pat. 5,565,332. In another embodiment, antibody chains or specific binding pair members may be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pats. 5,565,332, 5,871,907, or 5,733,743. Humanized antibodies of the present invention may be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell.

The humanized antibody expression vector may be either of a type in which a gene encoding an antibody heavy chain and a gene encoding an antibody light chain exists on separate vectors or of a type in which both genes exist on the same vector (tandem type).

Methods for producing humanized antibodies based on conventional recombinant DNA and gene transfection techniques are well known in the art (See, e.g., Riechmann L. et al. 1988; Neuberger M S. et al. 1985). Antibodies may be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan EA (1991); Studnicka GM et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

Similarly, bispecific or multispecific antibodies described herein may be made according to standard procedures. For example, triomas and hybrid hybridomas are two examples of cell lines that may secrete bispecific or multispecific antibodies. Examples of bispecific and multispecific antibodies produced by a hybrid hybridoma or a trioma are disclosed in U.S. Pat. 4,474,893. Such antibodies may also be constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Alternatively, such antibodiesmay also be generated by making heterohybridomas by fusing hybridomas or other cells making different antibodies, followed by identification of clones producing and co-assembling the desired antibodies. They may also be generated by chemical or genetic conjugation of complete immunoglobulin chains or portions thereof such as Fab and Fv sequences. The antibody component may bind to a polypeptide or a fragment thereof of one or more biomarkers of the invention, including one or more immunoinhibitory biomarkers described herein.

In addition, methods for producing antibody fragments are well known. For example, Fab fragments of the present invention may be obtained by treating an antibody with a protease, papaine. Also, Fabs may be produced by inserting DNA encoding Fabs of the antibody into a vector for prokaryotic expression system, or for eukaryotic expression system, and introducing the vector into a procaryote or eucaryote (as appropriate) to express the Fabs.

Similarly, F(ab′)2 fragments of the present invention may be obtained treating an antibody with a protease, pepsin. Also, the F(ab′)2 fragment may be produced by binding Fab′ described below via a thioether bond or a disulfide bond.

Fab′ fragments of the present invention may be obtained treating F(ab′)2 with a reducing agent, dithiothreitol. Also, the Fab′ fragments may be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present invention may be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well known technology called CDR grafting may be used, which involves selecting the complementary determining regions (CDRs) from a donor scFv fragment, and grafting them onto a human scFv fragment framework of known three dimensional structure (see, e.g., WO98/45322; WO 87/02671; U.S. Pat. No. 5,859,205; U.S. Pat. No. 5,585,089; U.S. Pat. No. 4,816,567; EP0173494).

### V. Modification of Bispecific Molecules, Antibodies, Immunoglobulins, and Polypeptides

Amino acid sequence modification(s) of the bispecific molecules and antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and may be corrected by replacing the amino acids with amino acid residues of the original antibody derived from a non-human animal.

Modifications and changes may be made in the structure of the antibodies of the present invention, and in the DNA sequences encoding them, and still obtain a functional molecule that encodes an antibody and polypeptide with desirable characteristics. For example, certain amino acids may be substituted by other amino acids in a protein structure without appreciable loss of activity. Since the interactive capacity and nature of a protein define the protein’s biological functional activity, certain amino acid substitutions may be made in a protein sequence, and, of course, in its DNA encoding sequence, while nevertheless obtaining a protein with like properties. It is thus contemplated that various changes may be made in the antibodies sequences of the invention, or corresponding DNA sequences which encode said polypeptides, without appreciable loss of their biological activity.

In one embodiment, amino acid changes may be achieved by changing codons in the DNA sequence to encode conservative substitutions based on conservation of the genetic code. Specifically, there is a known and definite correspondence between the amino acid sequence of a particular protein and the nucleotide sequences that may code for the protein, as defined by the genetic code (shown below). Likewise, there is a known and definite correspondence between the nucleotide sequence of a particular nucleic acid and the amino acid sequence encoded by that nucleic acid, as defined by the genetic code.

| | |
|---|---|
| GENETIC CODE | |
| Alanine (Ala, A) | GCA, GCC, GCG, GCT |
| Arginine (Arg, R) | AGA, ACG, CGA, CGC, CGG, CGT |
| Asparagine (Asn, N) AAC, AAT | |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |

| | |
|---|---|
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (-0.4); threonine (-0.7); serine (-0.8); tryptophane (-0.9); tyrosine (-1.3); proline (-1.6); histidine (-3.2); glutamate (-3.5); glutamine (-3.5); aspartate (<RTI 3.5); asparagine (-3.5); lysine (-3.9); and arginine (-4.5).

It is known in the art that certain amino acids may be substituted by other amino acids having a similar hydropathic index or score and still result in a protein with similar biological activity, i.e., still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

Another type of amino acid modification of the antibody of the invention may be useful for altering the original glycosylation pattern of the antibody to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies may be achieved by the use of a variety of endo- and exo-glycosidases as described by Thotakura, N R. et al. (1987).

Other modifications may involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

Conjugation of antibodies or other proteins of the present invention with heterologous agents may be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6 diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features antibodies conjugated to a therapeutic moiety, such as a cytotoxin, a drug, and/or a radioisotope. When conjugated to a cytotoxin, these antibody conjugates are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). An antibody of the present invention may be conjugated to a radioisotope, e.g., radioactive iodine, to generate cytotoxic radiopharmaceuticals for treating a related disorder, such as a cancer.

Conjugated antibodies may be used diagnostically or prognostically to monitor polypeptide levels in tissue as part of a clinical testing procedure, e.g., to determine the efficacy of a given treatment regimen or to select patients most likely to response to an immunotherapy. For example, cells may be permeabilized in a flow cytometry assay to allow antibodiesto bind to their epitopes and allow detection of the binding by analyzing signals emanating from the conjugated molecules. Detection may be facilitated by coupling (ie., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. [0134] As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or indocyanine (Cy5)) to the antibody, as well as indirect labeling of the antibody by reactivity with a detectable substance.

The antibody conjugates of the present invention may be used to modify a given biological response. The therapeutic moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other cytokines or growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243 56 (Alan R. Liss, Inc. 1985); Hellstrom et al., “Antibodies For Drug Delivery”, in Controlled Drug Delivery (2nd Ed.), Robinson et al. (eds.), pp. 623 53 (Marcel Dekker, Inc. 1987); Thorpe, “Antibody Carriers Of Cytotoxic Agents In Cancer Therapy: A Review”, in Monoclonal Antibodies ‘84: Biological And Clinical Applications, Pinchera et al. (eds.), pp. 475 506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303 16 (Academic Press 1985), and Thorpe et al., “The Preparation And Cytotoxic Properties Of Antibody-Toxin Conjugates”, Immunol. Rev., 62:119 58 (1982).

In some embodiments, conjugations may be made using a “cleavable linker” facilitating release of the cytotoxic agent or growth inhibitory agent in a cell. For example, an acid-labile linker, peptidase-sensitive linker, photolabile linker, dimethyl linker or disulfide-containing linker (See e.g. U.S. Pat. No. 5,208,020) may be used. Alternatively, a fusion protein comprising the antibody and cytotoxic agent or growth inhibitory agent may be made, by recombinant techniques or peptide synthesis. The length of DNA may comprise respective regions encoding the two portions of the conjugate either adjacent one another or separated by a region encoding a linker peptide which does not destroy the desired properties of the conjugate.

### VI. Uses and Methods of the Invention

The antibodies, immunoglobulins, polypeptides, and nucleic acids of the present invention described herein may be used in the treatment of numerous conditions, as further described below.

### A. Solid Organ Transplantation

Pharmacologic immunosuppressants are used to prevent the rejection of solid organ allografts. These agents inhibit T cell activity not only at the site of the allograft but throughout the body, and as a result, transplant recipients are at an increased risk of infection and cancer. Existing immunosuppressants may also result in cumulative toxicity and impair organ function, which may limit their dosing. Organ rejection may still occur despite treatment with existing agents.

When a recipient receives a transplanted organ graft with at least one mismatched allele of either MHC class I or MHC class II, the cells in that graft express a surface antigen that is not present on any other tissues in the body of the recipient. A LoSTIM (i.e., bispecific molecule, as used in this disclosure) with specific avidity for the protein product of that mismatched MHC allele would inhibit T cell activity against the cells of the allograft but not the activity of T cells elsewhere in the recipient’s body. Thus, a LoSTIM would not be expected to increase the risk of infection or cancer in parts of the recipient’s body outside of the allograft. LoSTIMs could serve as either replacements for existing immunosuppressants or as adjunctive agents, allowing existing immunosuppressants to be used at a lower dose with reduced toxicity.

### B. Autoimmune Disease

Generally speaking, autoimmune diseases result in pathologic inflammation of a subset of tissue types rather than involving all tissues in the body. Some examples of this include rheumatoid arthritis, colitis, Chron’s disease, autoimmune hepatitis, lupus, and type I diabetes. Current pharmacologic immunosuppressants used to treat autoimmune diseases, however, inhibit immune cell activity throughout the body. This systemic immunosuppression places patients at increased risk of infection and cancer and current agents are sometimes unable to adequately suppress autoimmunity.

A LoSTIM with specific avidity for antigens expressed on the surface of pathologically inflamed tissues would inhibit T cell activity at that location but not elsewhere in the body. Thus, a LoSTIM would be expected to treat autoimmune conditions mediated by T cells, but not to increase the risk of infection or cancer in parts of the body that are not targeted for immunosuppression by the LoSTIM.

### C. Treatment of Cancer: Checkpoint-Inhibitor Immune Related Adverse Effects

Immune-related adverse effects (irAE) occur in a significant proportion of patients receiving immunotherapies that inhibit the PD-1/PDL1 signaling axis, and on occasion, these adverse effects may be severe. The most common irAE include pneumonitis, colitis, hepatitis, dermatitis, thyroiditis, and arthritis, but autoimmunity may be induced in almost any tissue. Management typically involves withdrawal of the immunotherapy and treatment with steroids. Some patients are not able to be tapered off of steroids and most patients are not re-challenged with immunotherapy out of concern for recurrent irAE, even if they were enjoying a response to therapy. Additionally, there is a possibility that the use of steroids to combat irAE may actually dampen the anti-tumor effect in patients who otherwise might demonstrate a continued response to PD-1 blockade.

A LoSTIM or panel of LoSTIMs directed against a surface antigen(s) expressed on tissues susceptible to irAE would inhibit T cell activity on those tissues while allowing productive T cell activity to persist elsewhere, most importantly at sites of tumor. This is provided, of course, that the tumor does not express antigens that bind the LoSTIM. Moreover, a LoSTIM that inhibits the interaction of PD-1 with its natural ligands would simultaneously serve as a PD-1 antagonist on other tissues, including at the sites of tumor. An appropriately designed LoSTIM, therefore, might treat or prevent irAE while continuing to provide treatment for cancer. Such a LoSTIM or panel of LoSTIMs could also be used as an upfront alternative to existing PD-1/PDL1 inhibitors in patients at risk for irAE or in all patients.

### D. Treatment of Cancer: Engineered Immune Cell Therapy

Engineered immune cell therapy in the form of chimeric antigen receptor T cells (CAR-T) is a recent addition to the armamentarium of therapies for hematologic malignancies including Bcell lymphoma, acute lymphocytic leukemia, and multiple myeloma. Progress has been slow, however, to translate this technology to solid malignancies. One of the main barriers to the use of CAR-T for solid cancers are so-called “on-target, off-tumor” effects, whereby CAR-T cells attack normal tissues that express the tumor-associated antigen targeted by the CAR.

Normal tissues that express the tumor-associated antigen target by the CAR could be protected by a LoSTIM or panel of LoSTIMs directed against surface antigens on those tissues. As long as the antigens to which the LoSTIM binds are not expressed on the cancer, the activity of the CAR-T against the tumor would be preserved. The LoSTIMs could be administered exogenously as a pharmaceutical agent or be expressed by the CAR-T cells themselves. Moreover, it has been shown that PD-1 blockade may enhance the activity of CAR-T cells against cancer. Therefore, LoSTIMs that block the interaction of PD-1 with its natural ligands could be used to potentiate the effect of the CAR-T cells against the cancer.

### E. Treatment of Cancer: Graft-Versus-Host Disease and Bone Marrow Transplantation

Graft-versus-host disease (GVHD), in both the acute and chronic setting, is a complication of allogeneic bone marrow transplantation. The recognition of major and minor alloantigens by transplanted T cells mediates this phenomenon, but it also mediates the so-called “graftversus- leukemia” (GVL) effect in which these lymphocytes attack residual leukemia cells, thus providing the majority of the long-term survival benefit of allogeneic transplants. There has been considerable effort to develop agents or methods to enhance the GVL effect while simultaneously dampening GVHD, but since these effects are likely mediated by the same lymphocytes, this remains an ongoing challenge for the field.

A LoSTIM or panel of LoSTIMs directed against surface antigens expressed on normal tissues susceptible to GVHD could treat or prevent GVHD. Importantly, and in contrast to existing immunosuppressive agents, the LoSTIM would preserve the GVL effect, as long as the LoSTIM does not bind to antigens on the cancerous cells. In fact, a LoSTIM that blocks the interaction of PD-1 with its natural ligands may simultaneously potentiate the GVL effect while protecting normal tissues from GVHD.

### VII. Further Uses and Methods of the Invention

The antibodies, immunoglobulins, polypeptides, and nucleic acids of the present invention described herein may be used in numerous predictive medicine assays (e.g., diagnostic assays, prognostic assays, and monitoring clinical trials) and, in some embodiments and may be useful for therapeutic purposes (e.g., therapeutic and prophylactic) either alone or when conjugated to toxic compounds or other therapeutics. The term “detection” as used herein includes qualitative and/or quantitative detection (measuring levels) with or without reference to a control. As described herein, some antibodies or fragments thereof of the present invention have one or more of the following activities: 1) bind to and/or modulate the activity of their natural binding partner(s), such as PD-L1 or PD-L2; 2) modulate intra- or intercellular signaling, such as co-immunoinhibitory signaling; 3) modulate activation and/or proliferation of lymphocytes; 4) modulate the immune response of an organism, e.g., a mammalian organism, such as a mouse or human; and 5) modulate immune cell anergy.

Thus, one aspect of the present invention relates to diagnostic assays for determining polypeptide levels in the context of a biological sample (e.g., blood, serum, cells, or tissue) to thereby determine the level of the polypeptide in the sample, to determine whether an individual is afflicted with a disorder and/or to determine the state of such a disorder, indicated by such levels.

The present invention also provides for prognostic (or predictive) assays for determining whether an individual is at risk of developing such a disorder. Another aspect of the present invention pertains to monitoring the influence of agents (e.g., drugs, compounds) on the expression or activity of the detected polypeptides in clinical trials.

The present invention also provides for detection of PD-1 as a means to identify agents that transduce a PD-1 signal. Agents that transduce a PD-1 signal would attenuate immune responses and might be useful in autoimmune diseases, asthma, and for the establishment of tolerance.

In any method described herein, PD-1 may be detected either alone or in combination with the expression of other molecules, such as other immune checkpoint and/or costimulatory molecules. Combinatorial detection (e.g., sequentially or simultaneously) of several molecules may provide useful information regarding synergies of therapeutic intervention and/or personalized, higher-resolution diagnoses of disorder subtypes. In some embodiments, PD-1 is combinatorially detected with one more markers.

### A. Diagnostic Assays

The present invention provides, in part, methods, systems, and code for detecting levels of a polypeptide.

An exemplary method for detecting the level of a polypeptide involves obtaining a biological sample from a test subject and contacting the biological sample with an antibody or antigen-binding fragment thereof of the present invention capable of detecting the polypeptide such that the level of the polypeptide is detected in the biological sample. In some embodiments, at least one antibody or antigen-binding fragment thereof is used, wherein two, three, four, five, six, seven, eight, nine, ten, or more such antibodies or antibody fragments may be used in combination (e.g., in sandwich ELISAs) or in serial. In certain instances, the statistical algorithm is a single learning statistical classifier system. For example, a single learning statistical classifier system may be used to classify a sample as a PD-1 sample based upon a prediction or probability value and the presence or level of PD-1. The use of a single learning statistical classifier system typically classifies the sample as a PD-1 sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the PD-1 sample classification results to a clinician, e.g., a histopathologist or an oncologist.

In another embodiment, the method of the present invention further provides a diagnosis in the form of a probability that the individual has a condition or disorder associated with aberrant PD-1. For example, the individual may have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having the condition or disorder. In yet another embodiment, the method of the present invention further provides a prognosis of the condition or disorder in the individual. In some instances, the method of classifying a sample as a PD-1 sample is further based on the symptoms (e.g., clinical factors) of the individual from which the sample is obtained. The symptoms or group of symptoms may be, for example, lymphocyte count, white cell count, erythrocyte sedimentation rate, diarrhea, abdominal pain, cramping, fever, anemia, weight loss, anxiety, depression, and combinations thereof. In some embodiments, the diagnosis of an individual as having a condition or disorder associated with aberrant PD-1 is followed by administering to the individual a therapeutically effective amount of a drug useful for treating one or more symptoms associated with the condition or disorder (e.g., chemotherapeutic agents).

In one embodiment, the methods further involve obtaining a control biological sample (e.g., biological sample from a subject who does not have a condition or disorder), a biological sample from the subject during remission or before developing a condition or disorder, or a biological sample from the subject during treatment for developing a condition or disorder.

An exemplary method for detecting the presence or absence of polypeptide or fragments thereof is an antibody of the present invention, or fragment thereof, capable of binding to a polypeptide, preferably an antibody with a detectable label. Antibodies may be polyclonal, or more preferably, monoclonal. Such agents may be labeled. The term “labeled”, with regard to the antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody. The term “biological sample” is intended to include tissues, cells, and biological fluids isolated from a subject, such as serum, as well as tissues, cells, and fluids present within a subject. That is, the detection method of the present invention may be used to detect a polypeptide, or fragments thereof, in a biological sample in vitro as well as in vivo. In vitro techniques for detection of polypeptide include enzyme linked immunosorbent assays (ELISAs), Western blots, immunoprecipitations, immunohistochemistry (IHC), intracellular flow cytometry and related techniques, and immunofluorescence. Furthermore, in vivo techniques for detection of a polypeptide or a fragment thereof include introducing into a subject a labeled anti-polypeptide antibody. For example, the antibody may be labeled with a radioactive, luminescent, fluorescent, or other similar marker whose presence and location in a subject may be detected by standard imaging techniques, either alone or in combination with imaging for other molecules, such as markers of cell type (e.g., CD8+ T cell markers).

In one embodiment, the biological sample contains polypeptide molecules from the test subject. A preferred biological sample is a serum, tumor microenvironment, peritumoral, or intratumoral, isolated by conventional means from a subject.

In another embodiment, the methods further involve obtaining a control biological sample from a control subject, contacting the control sample with a compound or agent capable of detecting polypeptide, or fragments thereof, such that the presence of polypeptide, or fragments thereof, is detected in the biological sample, and comparing the presence of polypeptide, or fragments thereof, in the control sample with the presence of polypeptide, or fragments thereof in the test sample.

In still other embodiments, the antibodies may be associated with a component or device for the use of the antibodies in an ELISA or RIA. Non-limiting examples include antibodies immobilized on solid surfaces for use in these assays (e.g., linked and/or conjugated to a detectable label based on light or radiation emission as described above). In other embodiments, the antibodies are associated with a device or strip for detection of PD-1 by use of an immunochromatographic or immunochemical assay, such as in a “sandwich” or competitive assay, immunohistochemistry, immunofluorescence microscopy, and the like. Additional examples of such devices or strips are those designed for home testing or rapid point of care testing. Further examples include those that are designed for the simultaneous analysis of multiple analytes in a single sample. For example, an unlabeled antibody of the invention may be applied to a “capture” PD-1 polypeptides in a biological sample and the captured (or immobilized) PD-1 polypeptides may be bound to a labeled form of an anti-PD-1 antibody of the invention for detection. Other standard embodiments of immunoassays are well known the skilled artisan, including assays based on, for example, immunodiffusion, immunoelectrophoresis, immunohistopathology, immunohistochemistry, and histopathology.

### B. Prognostic Assays

The diagnostic methods described herein may furthermore be utilized to identify subjects having or at risk of developing a disorder. Aberrant expression or activity includes increased or decreased expression or activity, as well as expression or activity which does not follow the wild type developmental pattern of expression or the subcellular pattern of expression. For example, aberrant PD-1 levels is intended to include the cases in which a mutation in the PD-1 gene or regulatory sequence, or amplification of the chromosomal PD-1 gene, thereof causes the PD-1 gene to be under-expressed or over-expressed and situations in which such mutations result in a non-functional PD-1 polypeptide or a polypeptide which does not function in a wild-type fashion, e.g., a polypeptide missing an intracellular domain and thus not able to interact with a PD-1 binding or signal partner. As used herein, the term “unwanted” includes an unwanted phenomenon involved in a biological response such as immune cell activation. For example, the term unwanted includes a PD-1 variant which is undesirable in a subject.

Many disorders associated with aberrant PD-1 are known to the skilled artisan, as explained further in the rest of the disclosure. PD-1 is expressed by multiple tumor types, including select lymphoid malignancies, virally-induced cancers, and many solid tumors. However, immunoinhibition is desired for downregulating immune responses in treating a number of disorders, such as autoimmune diseases, inflammatory diseases, and the like.

The assays described herein, such as the preceding diagnostic assays or the following assays, may be utilized to identify a subject having or at risk of developing a disorder associated with a misregulation of PD-1 activation. Thus, the present invention provides a method for identifying a disorder associated with aberrant or unwanted PD-1 activation in which a test sample is obtained from a subject and a PD-1 level is detected, wherein the presence of an uberrant or unwanted PD-1 polypeptide is diagnostic for a subject having or at risk of developing the disorder associated with aberrant or unwanted PD-1 activity. As used herein, a “test sample” refers to a biological sample obtained from a subject of interest. For example, a test sample may be a biological fluid (e.g., cerebrospinal fluid or serum), cell sample, or tissue, such as a histopathological slide of the tumor microenvironment, peritumoral area, and/or intratumoral area.

Furthermore, the prognostic assays described herein may be used to determine whether a subject may be administered an agent (e.g., an agonist, antagonist, peptidomimetic, polypeptide, peptide, nucleic acid, small molecule, or other drug candidate) to treat such a disorder associated with aberrant or unwanted PD-1 activity. For example, such methods may be used to determine whether a subject may be effectively treated with one or a combination of agents. Thus, the present invention provides methods for determining whether a subject may be effectively treated with one or more agents for treating a disorder associated with aberrant or unwanted PD-1 activation.

The methods described herein may be performed, for example, by utilizing prepackaged diagnostic kits comprising at least one antibody reagent described herein, which may be conveniently used, e.g., in clinical settings to diagnose patients exhibiting symptoms or family history of a disease or illness involving PD-1.

Furthermore, any cell type or tissue in which PD-1 is expressed may be utilized in the prognostic assays described herein.

Another aspect of the present invention includes uses of the compositions and methods described herein for association and/or stratification analyses in which PD-1 in biological samples from individuals with a disorder associated with aberrant PD-1 activation, are analyzed and the information is compared to that of controls (e.g., individuals who do not have the disorder; controls may be also referred to as “healthy” or “normal” individuals or at early timepoints in a given time lapse study) who are preferably of similar age and race. The appropriate selection of patients and controls is important to the success of association and/or stratification studies. Therefore, a pool of individuals with well-characterized phenotypes is extremely desirable. Criteria for disease diagnosis, disease predisposition screening, disease prognosis, determining drug responsiveness (pharmacogenomics), drug toxicity screening, etc. are described herein.

Different study designs may be used for genetic association and/or stratification studies (Modern Epidemiology, Lippincott Williams & Wilkins (1998), 609-622). Observational studies are most frequently carried out in which the response of the patients is not interfered with. The first type of observational study identifies a sample of persons in whom the suspected cause of the disease is present and another sample of persons in whom the suspected cause is absent, and then the frequency of development of disease in the two samples is compared. These sampled populations are called cohorts, and the study is a prospective study. The other type of observational study is case-control or a retrospective study. In typical case-control studies, samples are collected from individuals with the phenotype of interest (cases) such as certain manifestations of a disease, and from individuals without the phenotype (controls) in a population (target population) that conclusions are to be drawn from. Then the possible causes of the disease are investigated retrospectively. As the time and costs of collecting samples in case-control studies are considerably less than those for prospective studies, case-control studies are the more commonly used study design in genetic association studies, at least during the exploration and discovery stage.

After all relevant phenotypic and/or genotypic information has been obtained, statistical analyses are carried out to determine if there is any significant correlation between the presence of an allele or a genotype with the phenotypic characteristics of an individual. Preferably, data inspection and cleaning are first performed before carrying out statistical tests for genetic association. Epidemiological and clinical data of the samples may be summarized by descriptive statistics with tables and graphs well known in the art. Data validation is preferably performed to check for data completion, inconsistent entries, and outliers. Chi-squared tests and t-tests (Wilcoxon rank-sum tests if distributions are not normal) may then be used to check for significant differences between cases and controls for discrete and continuous variables, respectively.

An important decision in the performance of genetic association tests is the determination of the significance level at which significant association may be declared when the p-value of the tests reaches that level. In an exploratory analysis where positive hits will be followed up in subsequent confirmatory testing, an unadjusted p-value <0.2 (a significance level on the lenient side), for example, may be used for generating hypotheses for significant association of a PD-1 level with certain phenotypic characteristics of a disease. It is preferred that a p-value <0.05 (a significance level traditionally used in the art) is achieved in order for the level to be considered to have an association with a disease. When hits are followed up in confirmatory analyses in more samples of the same source or in different samples from different sources, adjustment for multiple testing will be performed as to avoid excess number of hits while maintaining the experiment-wise error rates at 0.05. While there are different methods to adjust for multiple testing to control for different kinds of error rates, a commonly used but rather conservative method is Bonferroni correction to control the experiment-wise or family-wise error rate (Multiple comparisons and multiple tests, Westfall et al, SAS Institute (1999)). Permutation tests to control for the false discovery rates, FDR, may be more powerful (Benjamini and Hochberg, Journal of the Royal Statistical Society, Series B 57, 1289-1300, 1995, Resampling-based Multiple Testing, Westfall and Young, Wiley (1993)). Such methods to control for multiplicity would be preferred when the tests are dependent and controlling for false discovery rates is sufficient as opposed to controlling for the experiment-wise error rates.

Once individual risk factors, genetic or non-genetic, have been found for the predisposition to disease, a classification/prediction scheme may be set up to predict the category (for instance, disease or no-disease) that an individual will be in depending on his phenotype and/or genotype and other non-genetic risk factors. Logistic regression for discrete trait and linear regression for continuous trait are standard techniques for such tasks (Applied Regression Analysis, Draper and Smith, Wiley (1998)). Moreover, other techniques may also be used for setting up classification. Such techniques include, but are not limited to, MART, CART, neural network, and discriminant analyses that are suitable for use in comparing the performance of different methods (The Elements of Statistical Learning, Hastie, Tibshirani & Friedman, Springer (2002)).

### C. Monitoring of Effects During Clinical Trials

Monitoring the influence of agents (e.g., compounds, drugs or small molecules) on the levels of a PD-1 polypeptide or a fragment thereof (e.g., the modulation of cell proliferation and/or migration) may be applied not only in basic drug screening, but also in clinical trials. For example, the effectiveness of an agent determined by a screening assay as described herein to decrease PD-1 gene expression, polypeptide levels, or downregulate PD-1 activity, may be monitored in clinical trials of subjects exhibiting decreased PD-1 gene expression, polypeptide levels, or downregulated PD-1 activity, or may be monitored in clinical trails of subjects exhibiting decreased PD-1 levels, detectable by the anti-PD-1 antibodies or fragments described herein. In such clinical trials, the expression or activity of a PD-1 gene and/or symptoms or markers of the disorder of interest, may be used as a “read out” or marker of the phenotype of a particular cell, tissue, or system. Similarly, the effectiveness of an agent determined by a screening assay as described herein to increase PD-1 gene expression, polypeptide levels, or increase PD-1 activity, may be monitored in clinical trials of subjects exhibiting increased PD-1 gene expression, polypeptide levels, or increased PD-1 activity. In such clinical trials, the expression or activity of a PD-1 gene and/or symptoms or markers of the disorder of interest, may be used as a “read out” or marker of the phenotype of a particular cell, tissue, or system, such as for an autoimmune disorder.

For example, and not by way of limitation, genes, including PD-1, that are modulated in cells by treatment with an agent (e.g., compound, drug or small molecule) which modulates PD-1 activity (e.g., identified in a screening assay as described herein) may be identified. Thus, to study the effect of agents on a disorder associated with aberrant PD-1 activation, for example, in a clinical trial, cells may be isolated and nucleic acids and/or protein prepared and analyzed for the levels of PD-1 and/or other genes implicated in the disorder associated with aberrant PD-1 activation. The levels of gene expression (e.g., a gene expression pattern) analyzed by measuring the amount of polypeptide produced, by one of the methods as described herein, or by measuring the levels of PD-1 or other genes. In this way, the gene expression pattern may serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, polypeptide, peptide, nucleic acid, small molecule, or other drug candidate identified by the screening assays described herein) including the steps of (i) obtaining a pre-administration sample from a subject prior to administration of the agent; (ii) detecting the level of PD-1 polypeptides, or fragments thereof, in the preadministration sample; (iii) obtaining one or more post-administration samples from the subject; (iv) detecting the level of PD-1 polypeptides, or fragments thereof, in the post-administration samples; (v) comparing the level of the PD-1 polypeptide, or fragments thereof, in the pre-administration sample with the PD-1 polypeptide in the post administration sample or samples; and (vi) altering the administration of the agent to the subject accordingly. For example, increased administration of the agent may be desirable to decrease the PD-1 to lower levels than detected, i.e., to increase the effectiveness of the agent. According to such an embodiment, PD-1 levels may be used as an indicator of the effectiveness of an agent, even in the absence of an observable phenotypic response. Similarly, PD-1 level analysis, such as by immunohistochemistry (IHC), may also be used to select patients who will receive PD-1 and/or PD-1 immunotherapy, or immunotherapy to inhibit one ore more immune checkpoints. Patients whose tumors having PD-1 activation are more likely to respond to PD-1 or PD-1 mAb immunotherapy, as describd herein. The immunotherapy will initially result in immune activation and the activated T cells will express IFN-gamma which in turn will upregulate PD-1 activation. Normally this would result in PD-1 engagement and down regulation of the immune response.

### D. Therapeutic Methods and Uses

In some embodiments, antibodies, fragments or immunoconjugates of the present invention (e.g., anti-PD-1 antibodies alone or conjugated to therapeutic moieties) are useful for treating any disorder (e.g., a cancer) associated with aberrant or undesired activation of PD-1. In certain embodiments, the treatment is of a mammal, such as a human. Such antibodies of the invention may be used alone or in combination with any suitable agent or appropriate therapy to treat the disorder of interest.

It is well known that therapeutic monoclonal antibodies may lead to the depletion of cells extracellularly bearing the antigen specifically recognized by the antibody. This depletion may be mediated through at least three mechanisms: antibody mediated cellular cytotoxicity (ADCC), complement dependent lysis, and direct anti-tumour inhibition of tumour growth through signals given via the antigen targeted by the antibody.

“Complement dependent cytotoxicity” or “CDC” refers to the lysis of a target cell in the presence of complement. Activation of the classical complement pathway is initiated by the binding of the first component of the complement system to antibodies which are bound to their cognate antigen. To assess complement activation, a CDC assay, e.g. as described in Gazzano-Santoro et al. (1997) may be performed.

“Antibody-dependent cell-mediated cytotoxicity” or “ADCC” refers to a form of cytotoxicity in which secreted antibodies bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell. To assess ADCC activity of a molecule of interest, an in vitro ADCC assay, such as that described in U.S. Pat. No. 5,500,362 or 5,821,337 may be performed. As is well known in the art, the Fc portions may be engineered to effect a desired interaction or lack thereof with Fc receptors.

For antibody-mediated binding, neutralization, and/or modulation of intracellular targets, certain modifications should be made. As described herein, certain antibody formats, such as sFvs and Fabs, are amenable to intracellular expression of antibody-like molecules. Methods of rmaking and using such adapted antibody-like molecules for targeting expression in different compartments of the cell, including the nucleus, ER, cytoplasm, golgi, plasma membrane, mitochondria, where they counteract antigens or molecules in a specific pathway, are well known (see, at least U.S. Pat. Publs. 2008-0233110 and 2003-0104402; Marasco et al. (1993) Proc. Natl. Acad. Sci. U.S.A. 90:7889-7893; Chen et al. (1994) Human Gene Therapy 5:595-601; Chen et al. (1994) Proc. Natl. Acad. Sci. U.S.A. 91:5932-5936; Mhashilkar et al. (1995) EMBO J. 14:1542-1551; Marasco et al. (1997) Gene Therapy 4:11-15; Richardson et al. (1995) Proc. Natl. Acad. Sci. U.S.A. 92:3137-3141; and Duan et al. (1994) Human Gene Therapy 5:1315-1324).

As used herein, the term “intracellular immunoglobulin molecule” is a complete immunoglobulin which is the same as a naturally-occurring secreted immunoglobulin, but which remains inside of the cell following synthesis. An “intracellular immunoglobulin fragment” refers to any fragment, including single-chain fragments of an intracellular immunoglobulin molecule. Thus, an intracellular immunoglobulin molecule or fragment thereof is not secreted or expressed on the outer surface of the cell. Single-chain intracellular immunoglobulin fragments are referred to herein as “single-chain immunoglobulins.” As used herein, the term “intracellular immunoglobulin molecule or fragment thereof” is understood to encompass an “intracellular immunoglobulin,” a “single-chain intracellular immunoglobulin” (or fragment thereof), an “intracellular immunoglobulin fragment,” an “intracellular antibody” (or fragment thereof), and an “intrabody” (or fragment thereof). As such, the terms “intracellular immunoglobulin,” “intracellular Ig,” “intracellular antibody,” and “intrabody” may be used interchangeably herein, and are all encompassed by the generic definition of an “intracellular immunoglobulin molecule, or fragment thereof.” An intracellular immunoglobulin molecule, or fragment thereof of the present invention may, in some embodiments, comprise two or more subunit polypeptides, e.g., a “first intracellular immunoglobulin subunit polypeptide” and a “second intracellular immunoglobulin subunit polypeptide.” However, in other embodiments, an intracellular immunoglobulin may be a “single-chain intracellular immunoglobulin” including only a single polypeptide. As used herein, a “single-chain intracellular immunoglobulin” is defined as any unitary fragment that has a desired activity, for example, intracellular binding to an antigen. Thus, single-chain intracellular immunoglobulins encompass those which comprise both heavy and light chain variable regions which act together to bind antigen, as well as single-chain intracellular immunoglobulins which only have a single variable region which binds antigen, for example, a “camelized” heavy chain variable region as described herein. An intracellular immunoglobulin or Ig fragment may be expressed anywhere substantially within the cell, such as in the cytoplasm, on the inner surface of the cell membrane, or in a subcellular compartment (also referred to as cell subcompartment or cell compartment) such as the nucleus, golgi, endoplasmic reticulum, endosome, mitochondria, etc. Additional cell subcompartments include those that are described herein and well known in the art.

Such intracellular immunoglobulins are expressed in a recipient cell or host cell containing the antigen to be targeted. A host cell of the present invention is preferably a eukaryotic cell or cell line, preferably a plant, animal, vertebrate, mammalian, rodent, mouse, primate, or human cell or cell line. The effects of modulating PD-1 signaling are well known in the art (see, for example, PCT Publ. WO 2001/014557).

In some embodiments, antibodies of the present invention may be conjugated to a therapeutic moiety, such as a growth inhibitory agent, cytotoxic agent, or a prodrug-activating enzyme as previously described. Antibodies of the invention may be useful for targeting said growth inhibitory agent, cytotoxic agent, or a prodrug to a cell that under- or over-expresses the desired amount of PD-1.

Thus, an object of the invention relates to a method for treating a disorder associated with aberrant PD-1 activation comprising administering a subject in need thereof with a therapeutically effective amount of an antibody, fragment or immunoconjugate of the present invention.

Alternatively, in some embodiments, the antibodies or the antigen-binding fragments of the present invention are useful for therapeutic applications, in addition to diagnostic, prognostic, and prevention applications regarding upregulating an immune response. Upregulation of immune responses may be in the form of enhancing an existing immune response or eliciting an initial immune response. For instance, enhancing an immune response using the subject compositions and methods is useful in cases of improving an immunological defense against cancer and infections with microbes (e.g., bacteria, viruses, or parasites). For example, upregulation or enhancement of an immune response function, as described herein, is useful in the induction of tumor immunity.

In another embodiment, the immune response may be stimulated by the methods described herein, such that preexisting tolerance, clonal deletion, and/or exhaustion (e.g., T cell exhaustion) is overcome. For example, immune responses against antigens to which a subject cannot mount a significant immune response, e.g., to an autologous antigen, such as a tumor specific antigens may be induced by administering appropriate agents described herein that upregulate the immune response. In one embodiment, an autologous antigen, such as a tumor-specific antigen, may be co-administered. In another embodiment, an immune response may be stimulated against an antigen (e.g., an autologous antigen) to treat a neurological disorder. In another embodiment, the subject agents may be used as adjuvants to boost responses to foreign antigens in the process of active immunization.

In certain instances, it may be desirable to further administer other agents that upregulate immune responses, for example, forms of other B7 family members that transduce signals via costimulatory receptors, in order to further augment the immune response. Also, agents that upregulate an immune response may be used prophylactically in vaccines against various polypeptides (e.g., polypeptides derived from pathogens). Immunity against a pathogen (e.g., a virus) may be induced by vaccinating with a viral protein along with an agent that upregulates an immune response, in an appropriate adjuvant.

Alternatively, in some embodiments, the antibodies and the antigen-binding fragments of the present invention are useful for therapeutic applications, in addition to diagnostic, prognostic, and prevention applications (such as treating, and delaying the onset or progression of the diseases), to inhibit diseases that upregulate the immune reaction, for example, asthma, autoimmune diseases (glomerular nephritis, arthritis, dilated cardiomyopathy-like disease, ulceous colitis, Sjogren syndrome, Crohn disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves’ disease, anaemia perniciosa, Goodpasture syndrome, sterility disease, chronic active hepatitis, pemphigus, autoimmune thrombopenic purpura, and autoimmune hemolytic anemia, active chronic hepatitis, Addison’s disease, anti-phospholipid syndrome, atopic allergy, autoimmune atrophic gastritis, achlorhydra autoimmune, celiac disease, Cushing’s syndrome, dermatomyositis, discoid lupus, erythematosis, Goodpasture’s syndrome, Hashimoto’s thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud’s syndrome, relapsing polychondritis, Schmidt’s syndrome, limited scleroderma (or crest syndrome), sympathetic ophthalmia, systemic lupus erythematosis, Takayasu’s arteritis, temporal arteritis, thyrotoxicosis, type b insulin resistance, ulcerative colitis and Wegener’s granulomatosis).

Similarly, the antibodies and the antigen-binding fragments of the present invention are useful for therapeutic applications, in addition to diagnostic, prognostic, and prevention applications (such as treating, and delaying the onset or progression of the diseases) for persistent infectious disease (e.g., viral infectious diseases including HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. Other antigens associated with pathogens that may be used as described herein are antigens of various parasites, includes malaria, preferably malaria peptide based on repeats of NANP. In addition, bacterial, fungal and other pathogenic diseases are included, such as Aspergillus, Brugia, Candida, Chlamydia, Coccidia, Cryptococcus, Dirofilaria, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum; or, a fungus, such as, e.g., Paracoccidioides brasiliensis; or other pathogen, e.g., Plasmodium falciparum. Also included are National Institute of Allergy and Infectious Diseases (NIAID) priority pathogens. These include Category A agents, such as variola major (smallpox), Bacillus anthracis (anthrax), Yersinia pestis (plague), Clostridium botulinum toxin (botulism), Francisella tularensis (tularaemia), filoviruses (Ebola hemorrhagic fever, Marburg hemorrhagic fever), arenaviruses (Lassa (Lassa fever), Junin (Argentine hemorrhagic fever) and related viruses); Category B agents, such as Coxiella burnetti (Q fever), Brucella species (brucellosis), Burkholderia mallei (glanders), alphaviruses (Venezuelan encephalomyelitis, eastern & western equine encephalomyelitis), ricin toxin from Ricinus communis (castor beans), epsilon toxin of Clostridium perfringens; Staphylococcus enterotoxin B, Salmonella species, Shigella dysenteriae, Escherichia coli strain 0157:H7, Vibrio cholerae, Cryptosporidium parvum; Category C agents, such as nipah virus, hantaviruses, tickborne hemorrhagic fever viruses, tickborne encephalitis viruses, yellow fever, and multidrug-resistant tuberculosis; helminths, such as Schistosoma and Taenia; and protozoa, such as Leishmania (e.g., L. mexicana) and Plasmodium.

In still another embodiment, the antibodies or the antigen-binding fragments of the present invention are useful for therapeutic applications, in addition to diagnostic, prognostic, and prevention applications regarding induction of immunological tolerance, organ graft rejection, graft-versus-host disease (GVHD), allergic disease, and diseases caused by attenuation of immune reactions mediated by PD-1.

In the context of the invention, the term “treating” or “treatment”, as used herein, means reversing, alleviating, inhibiting the progress of, or preventing the disorder or condition to which such term applies, or one or more symptoms of such disorder or condition. By the term “treating cancer” as used herein is meant the inhibition of the growth and/or proliferation of cancer cells. Preferably such treatment also leads to the regression of tumor growth (i.e., the decrease in size of a measurable tumor). Most preferably, such treatment leads to the complete regression of the tumor.

In some embodiments, the term “patient” or “patient in need thereof” is intended for a human or non-human mammal affected or likely to be affected with a cancer associated with aberrant activation of PD-1.

By a “therapeutically effective amount” of the polypeptide of the invention is meant a sufficient amount of the antibody to treat the disorder of interest, such as cancer, at a reasonable benefit/risk ratio applicable to any medical treatment. It will be understood, however, that the total daily usage of the antibodies and compositions of the present invention will be decided by the attending physician within the scope of sound medical judgment. The specific therapeutically effective dose level for any particular patient will depend upon a variety of factors including the disorder being treated and the severity of the disorder; activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved.

Therapeutic formulations comprising one or more antibodies of the invention are prepared for storage by mixing the antibody having the desired degree of purity with optional physiologically acceptable carriers, excipients or stabilizers (Remington’s Pharmaceutical Sciences 16th edition, Osol, A. Ed. (1980)), in the form of lyophilized formulations or aqueous solutions. The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The therapeutic dose may be at least about 0.01 µg/kg body weight, at least about 0.05 µg/kg body weight; at least about 0.1 µg/kg body weight, at least about 0.5 µg/kg body weight, at least about 1 µg/kg body weight, at least about 2.5 µg/kg body weight, at least about 5 µg/kg body weight, and not more than about 100 µg/kg body weight. It will be understood by one of skill in the art that such guidelines will be adjusted for the molecular weight of the active agent, e.g. in the use of antibody fragments, or in the use of antibody conjugates. The dosage may also be varied for localized administration, e.g. intranasal, inhalation, etc., or for systemic administration, e.g. i.m., i.p., i.v., and the like.

The composition need not be, but is optionally formulated with one or more agents that potentiate activity, or that otherwise increase the therapeutic effect. These are generally used in the same dosages and with administration routes as used hereinbefore or about from 1 to 99% of the heretofore employed dosages.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration should be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington’s Pharmaceutical Sciences 16th edition, Osol, A. Ed. (1980).

The compositions described herein may be administered by any suitable means, including parenteral, subcutaneous, intraperitoneal, intrapulmonary, and intranasal. Parenteral infusions include intramuscular, intravenous, intraarterial, intraperitoneal, or subcutaneous administration. In addition, the compositions may be suitably administered by pulse infusion, particularly with declining doses of the antibody.

For the prevention or treatment of disease, the appropriate dosage of antibody will depend on the type of disease to be treated, as defined above, the severity and course of the disease, whether the antibody is administered for preventive purposes, previous therapy, the patient’s clinical history and response to the antibody, and the discretion of the attending physician. The antibody is suitably administered to the patient at one time or over a series of treatments.

### E. Assays and Screening Methods

Another aspect of the present invention relates to screening assays, including non-cell based assays and xenograft animal model assays. In one embodiment, the assays provide a method for identifying agents that modulate PD-1 signaling, such as in a human or an animal model assay, in order to identify agents that reduce PD-1 signaling thereby increasing immune responses and/or identify agents that increase PD-1 signaling thereby decreasing immune responses.

In one embodiment, the present invention relates to assays for screening test agents which bind to, or modulate the biological activity of, at least one biomarker described herein (e.g., in the tables, figures, examples, or otherwise in the specification), such as PD-1. In one embodiment, a method for identifying such an agent entails determining the ability of the agent to modulate, e.g. inhibit, the at least one biomarker described herein.

In one embodiment, an assay is a cell-free or cell-based assay, comprising contacting at least one biomarker described herein, with a test agent, and determining the ability of the test agent to modulate (e.g., inhibit) the enzymatic activity of the biomarker, such as by measuring direct binding of substrates or by measuring indirect parameters as described below.

For example, in a direct binding assay, biomarker protein (or their respective target polypeptides or molecules) may be coupled with a radioisotope or enzymatic label such that binding may be determined by detecting the labeled protein or molecule in a complex. For example, the targets may be labeled with ¹²⁵I, ³⁵S, ¹⁴C, or ³H, either directly or indirectly, and the radioisotope detected by direct counting of radioemmission or by scintillation counting. Alternatively, the targets may be enzymatically labeled with, for example, horseradish peroxidase, alkaline phosphatase, or luciferase, and the enzymatic label detected by determination of conversion of an appropriate substrate to product. Determining the interaction between biomarker and substrate may also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target may be accomplished in any vessel suitable for containing the reactants. Non-limiting examples of such vessels include microtiter plates, test tubes, and micro-centrifuge tubes. Immobilized forms of the antibodies described herein may also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between the biomarker and a substrate or a biomarker and its natural binding partner may be accomplished by determining the ability of the test agent to modulate the activity of a polypeptide or other product that functions downstream or upstream of its position within the signaling pathway (e.g., feedback loops). Such feedback loops are well-known in the art (see, for example, Chen and Guillemin (2009) Int. J. Tryptophan Res. 2:1-19).

The present invention further pertains to novel agents identified by the above-described screening assays. Accordingly, it is within the scope of this invention to further use an agent identified as described herein, such as in an appropriate animal model. For example, an agent identified as described herein may be used in an animal model to determine the efficacy, toxicity, or side effects of treatment with such an agent. Alternatively, an antibody identified as described herein may be used in an animal model to determine the mechanism of action of such an agent.

### VIII. Kits

In addition, the present invention also encompasses kits for detecting the presence of a PD-1 polypeptide, or fragments thereof, in a biological sample. For example, the kit may comprise a labeled compound or agent capable of detecting a PD-1 polypeptide, or fragments thereof, in a biological sample; means for determining the amount of the PD-1 polypeptide, or fragments thereof, in the sample; and means for comparing the amount of the PD-1 polypeptide, or fragments thereof, in the sample with a standard. The compound or agent may be packaged in a suitable container. For example, the present invention provides kits comprising at least one antibody described herein. Kits containing antibodies of the invention find use in detecting PD-1, or in therapeutic or diagnostic assays. Kits of the invention may contain an antibody coupled to a solid support, e.g., a tissue culture plate or beads (e.g., sepharose beads).

A kit may include additional components to facilitate the particular application for which the kit is designed. For example, kits may be provided which contain antibodies for detection and quantification of PD-1 in vitro, e.g. in an ELISA or a Western blot. Additional, exemplary agents that kits may contain include means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or PD-1 protein standards). A kit may additionally include buffers and other reagents recognized for use in a method of the disclosed invention. Non-limiting examples include agents to reduce non-specific binding, such as a carrier protein or a detergent. A kit of the present invention may also include instructional materials disclosing or describing the use of the kit or an antibody of the disclosed invention in a method of the disclosed invention as provided herein.

This invention is further illustrated by the following examples which should not be construed as limiting. The contents of all references, patents and published patent applications cited throughout this application, as well as the Figures, are incorporated herein by reference.

### EXAMPLES

### Example 1: Selective Modulation of T Cell Activity Using Bispecific Molecules That Engage PD-1 and Function as Either Agonists or Antagonists

Disclosed is a method of inhibiting T cell activity using bispecific molecules that act as PD-1 agonists. These molecules bind PD-1 on the T cell surface and also bind an antigen on the opposing surface of an adjacent cell. This bispecific interaction allows them to cluster PD-1 on the T cell surface and colocalizes this cluster with the immunological synapse, thereby activating PD-1 and inhibiting T cell activity. Engagement of the antigen on the target cell surface is necessary for these molecules to function as PD-1 agonists, and this property may be exploited to create molecules that function as selective PD-1 agonists that inhibit T cell activity on some tissues but not others. This category of a molecule is referred to herein as a Location-Specific T cell Inhibitory Molecule (LoSTIM). A LoSTIM may also be designed to block the engagement of PD-1 by its natural ligands, in which case it will act as an antagonist of PD-1 on T cells interacting with tissues that do not bind the LoSTIM, while still acting as an agonist of PD-1 on T cells interacting with tissues that do bind the LoSTIM. A single LoSTIM molecule of that design will, therefore, function as either a potentiator or inhibitor of T cell function depending on the tissue context. By contrast, a LoSTIM that does not block the engagement of PD-1 by its natural ligands will function exclusively as a cell-specific and/or tissue-specific inhibitor of T cell function.

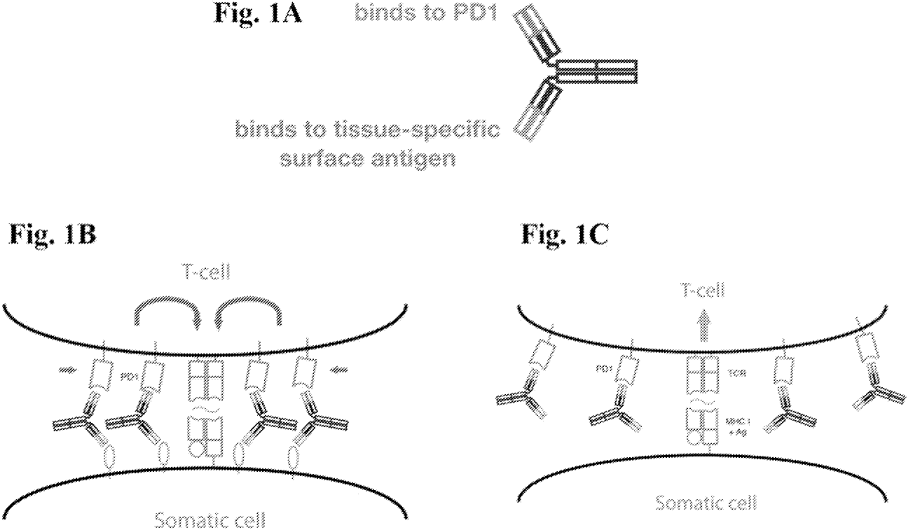

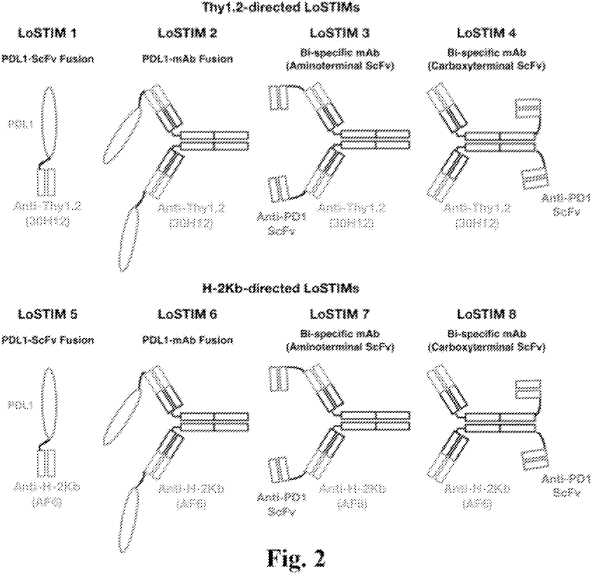

A schematic overview of exemplary LoSTIMs is shown in FIG. 1A to FIG. 1C. FIG. 1A shows a schematic representation of a LoSTIM. A LoSTIM is a molecule capable of simultaneously binding to PD-1 as well as to an arbitrary cell-specific and/or tissue-specific surface antigen expressed on tissues where inhibition of T cell activity is desired. FIG. 1B shows that an embodiment of a LoSTIM simultaneously binds PD-1 molecules on a T cell as well as molecules of an antigen on the surface of an opposing somatic cell, thereby clustering PD-1 on the T cell surface in proximity to an immunologic synapse between the two cells. This clustering and co-localization of PD-1 inhibits T cell activation. FIG. 1C shows that an embodiment of a LoSTIM binds PD-1 on the T cell but does not bind an antigen on the opposing somatic cell surface since its cognate antigen is not expressed by that cell. Because the LoSTIM is not fixed in place by binding to the opposing cell surface, it is not able to cluster PD-1 or co-localize PD-1 with the immunologic synapse on the T cell surface and therefore does not inhibit T cell activation. In this circumstance, if the PD-1 binding domain of the LoSTIM inhibits the engagement of PD-1 by its natural ligands, the LoSTIM acts as a potentiator of T cell activation.

Four generalized LoSTIM designs were evaluated in the subsequent studies: (1) a PDL1-scFv fusion protein in which the scFv binds to a cell-specific and/or tissue-specific surface antigen expressed on tissues where T cell inhibition is desired; (2) a PDL1-mAb fusion in which the mAb is directed against a cell-specific and/or tissue-specific surface antigen expressed on tissues where T cell inhibition is desired; and (3) & (4), bispecific monoclonal antibodies with avidity for PD-1 and for a cell-specific and/or tissue-specific surface antigen expressed on tissues where T cell inhibition is desired. FIG. 2 shows schematics of LoSTIMs used in these studies. LoSTIMs 1 through 4 have bispecific avidity for Thy1.2 (CD90.2) and mouse PD-1. The anti-Thy1.2 binding domain is the variable region of the anti-Thy1.2 clone AF6 and the anti-PD-1 binding domain is via the variable region of anti-PD-1 clone 1A12. LoSTIMs 5 through 8 have bispecific avidity for H-2Kb (a C57B6 class I MHC allele) and mouse PD-1. The anti-H-2Kb binding domain is the variable region of the anti-H-2Kb clone AF6. It should be noted that the anti-PD-1 clone 1A12 is known to block the interaction of PD-1 with its natural ligands and function as a PD-1 antagonist. The CH1, CH2, and CH3 domains of the antibody LoSTIMs contain several point mutations known to inhibit C1q and Fc gamma receptor binding, thus rendering these Fc domains “inert.”

The sequences of these LoSTIMs are presented in Example 2.

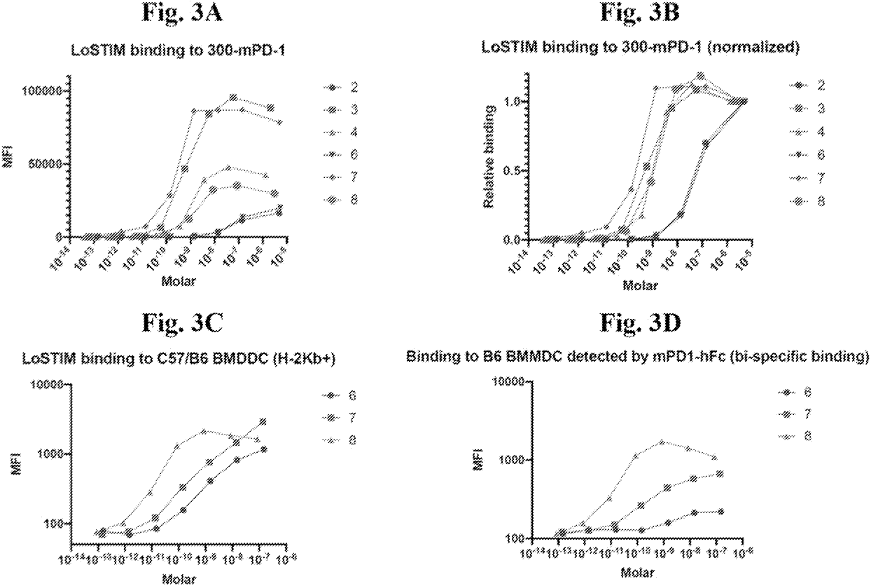

LoSTIM binding using these constructs was invenstigated, and some results are shown in FIG. 3A through FIG. 3D. As shown in FIG. 3A, 300-mPD-1 cells, transgenic mouse pro-B cells which overexpress mouse PD-1, were exposed to varying concentrations of LoSTIMs 2-4 and 6-8. Binding was detected via an anti-mIgG2a-PE conjugate. FIG. 3B shows data from panel A normalized to allow for a better assessment of relative PD-1 avidities. As shown in FIG. 3C, bone-marrow-derived dendritic cells (BMDCC) generated from C57B6 bone marrow cells cultured with GM-CSF for 8 days were exposed to varying concentrations of LoSTIMs 6-8. Binding was detected via an anti-mIgG2a-PE conjugate. As shown in FIG. 3D, BMDDC were exposed to varying concentrations of LoSTIMs 6-8. Bispecific binding was assessed by detecting the binding of a mouse PD-1-human Fc fusion protein. An anti-human IgG-PE conjugate was used as the detector.

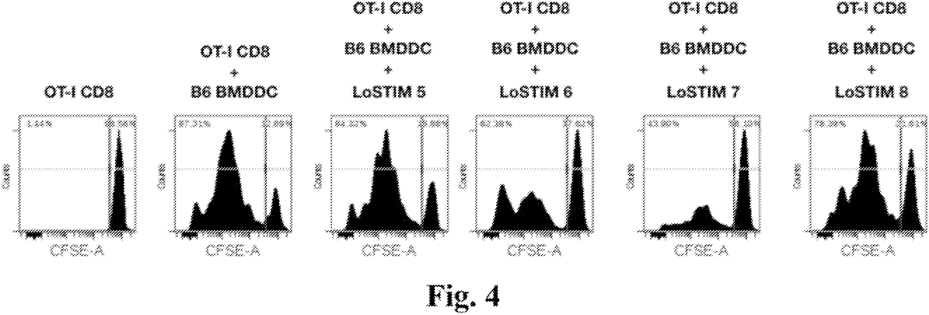

Further results are provided in FIG. 4, which shows that OT-I T cell activation by SIINFEKL-pulsed bone-marrow-derived dendritic cells (BMDDC) is inhibited by LoSTIMs. C57B6 bone marrow cells were cultured with GM-CSF for 8 days to yield BMDDC. These BMDDC were pulsed with 10 micromolar of Ser-Ile-Ile-Asn-Phe-Glu-Lys-Leu (SIINFEKL) peptide for 6 hours, washed, then co-cultured with CFSE-labeled OT-I CD8a+ T cells, which express a recombinant TCR that recognizes SIINFEKL in the context of H-2Kb. These T cells were obtained via negative magnetic selection from OT-I mouse splenocytes. The co-cultures were treated with 0.5 micromolar of the anti- H-2Kb-directed LoSTIMs 5, 6, 7, or 8. Cells were co-cultured for 72 hours. The histograms presented above represent gated CD8+ cells.

OT-I T cells cultured without BMDDC were inactive (1.4% proliferation by CFSE dilution). OT-I T cells stimulated with BMDDC were activated (87.3% proliferation by CFSE dilution). LoSTIM 5 had negligible effect on the activation OT-I T cells by BMDDC (84.3% proliferation by CFSE dilution). LoSTIM 6 inhibited OT-I T cell activation by BMDDC (62.4% proliferation by CFSE dilution). LoSTIM 7 inhibited OT-I T cell activation by BMDDC most potently (43.9% proliferation by CFSE dilution). LoSTIM 8 had a modest effect on OT-I T cell activation (78.4% proliferation by CFSE dilution).

This experiment demonstrates inhibition of T cell activity by LoSTIM molecules of various molecular designs. These molecules are all are capable of simultaneously binding PD-1 on a T cell and an antigen on the surface of a cell interacting with the T cell.

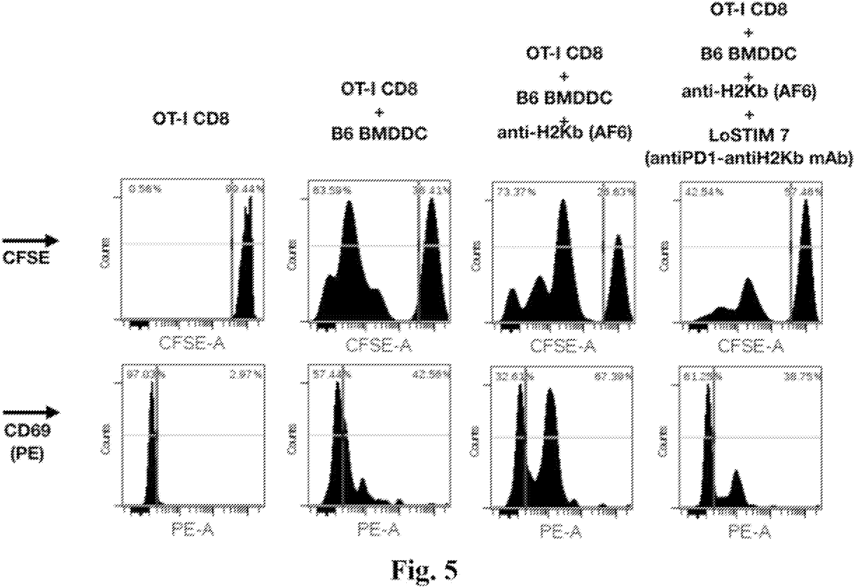

It has also been found that, as shown in FIG. 5, LoSTIM-mediated inhibition of OT-I T cell activation is not due to H-2Kb blockade and requires a LoSTIM that engages PD-1. To address whether inhibition of OT-I activation by SIINFEKL-pulsed BMDDC was simply due to blockade of H-2Kb antigen presentation by LoSTIMs, CFSE-labeled OT-I CD8a+ T cells were cocultured with BMDDC in the presence of 0.5 micromolar of anti-H-2Kb, clone AF6. The H-2Kb binding domains of LoSTIMs 5-8 are derived from this clone, so AF6 would be expected to bind the same epitope of H-2Kb as LoSTIMs 5-8. After 57 hours of co-culture, OT-I T cell activation was assessed by CFSE dilution and CD69 expression.

OT-I cells co-cultured with BMDDC in the absence of antibody treatment were activated (63.6% proliferation by CFSE dilution and 42.6% CD69 expression). The addition of anti-H-2Kb clone AF6 to such a co-culture did not inhibit T cell activation (73.4% proliferation by CFSE dilution and 67.4% CD69 expression). Addition of equimolar quantities of anti-H-2Kb clone AF6 and LoSTIM 7 to such a co-culture did result in some inhibition of T cell activation (42.5% proliferation by CFSE dilution and 38.8% CD69 expression).

In summary, treatment with anti-H-2Kb clone AF6 was not sufficient to inhibit T cell activation. Thus, LoSTIM-mediated inhibition of T cell activation was not simply due to blockade of H-2Kb antigen presentation. Moreover, an equimolar mixture of LoSTIM 7 and anti-H-2Kb clone AF6 was able to exert some inhibition on T cell activation. As LoSTIM 7 and clone AF6 possess the same H-2Kb binding domain, one would expect an equilibration of the two species on surface H-2Kb. T cell activation was inhibited in the co-culture that contained this 1:1 mixture of species, thus demonstrating that the presence of a LoSTIM molecule that both binds H-2Kb as well as PD-1 is necessary for T cell inhibition.

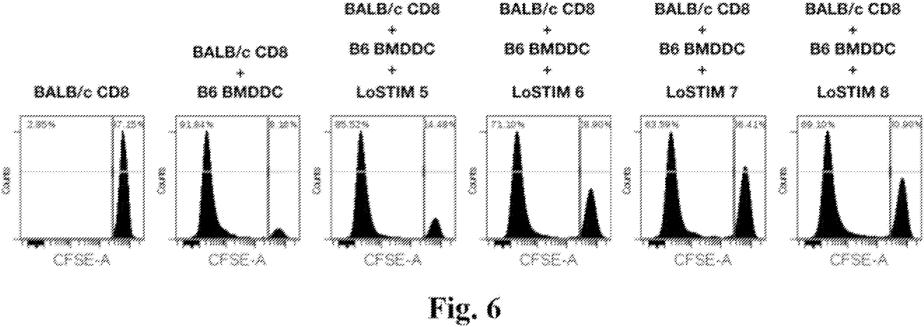

As the experimental results shown in FIG. 6 demonstrate, allogeneic T cell activation is inhibited by LoSTIM treatment. To address whether LoSTIM molecules can inhibit the activation of T cells by allogeneic cells, C57B6 BMDDC were co-cultured with CFSE-labeled BALB/c CD8 T cells for 84 hours in the presence of LoSTIMs 5-8 at 0.5 micromolar.

BALB/c CD8 T cells co-cultured with BMDDC derived from C57B6 mice were potently activated (91.8% proliferation by CFSE dilution). Treatment of such a co-culture with LoSTIM 5 had only a minimal inhibitory effect on T cell activation (85.5% proliferation by CFSE dilution). Treatment with LoSTIM 6 inhibited T cell activation (71.1% proliferation by CFSE dilution). Treatment with LoSTIM 7 resulted in the most potent inhibition of T cell activation (63.6% proliferation by CFSE dilution). Treatment with LoSTIM 8 also resulted in inhibition of T cell activation (69.1% proliferation by CFSE dilution).

In summary, LoSTIM molecules of various molecular designs are capable of inhibiting the allogeneic activation of T cells.

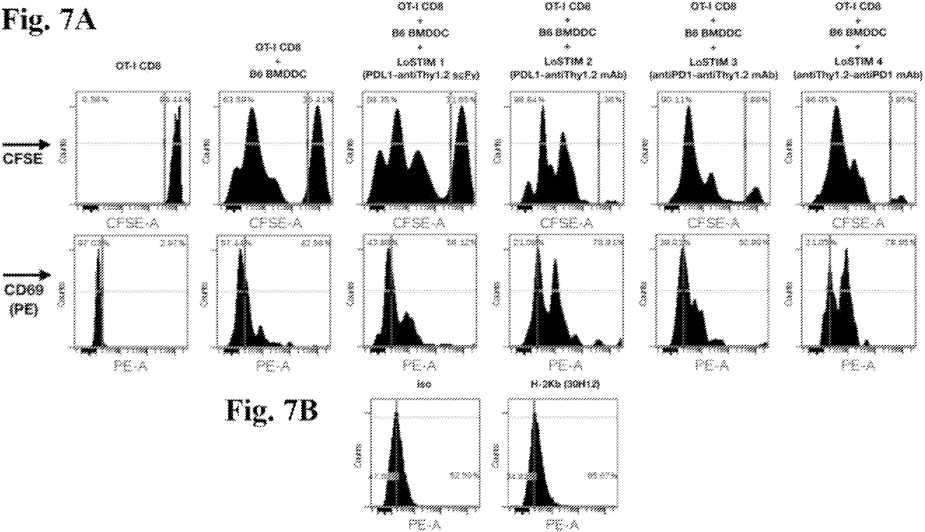

The experimental results shown in FIG. 7 indicate that a LoSTIM binds the surface of an interacting cell to inhibit T cell activation, and that a LoSTIM that inhibits the binding of PD-1 to its natural ligands will potentiate T cell activation if it does not also bind the surface of the interacting cell. To determine whether binding of a LoSTIM to the surface of an opposing cell is necessary for the inhibition of T cell activation, CFSE-labeled OT-I CD8 T cells were co-cultured with SIINFEKL-pulsed BMDDC in the presence of LoSTIMs 1-4 at 0.5 micromolar for 57 hours. LoSTIMs 1-4 are identical in molecular design to LoSTIMs 5-8 except that they carry a Thy1.2 (CD90.2) binding domain instead of an H-2Kb binding domain. Since BMDDCs do not express Thy1.2 (panel B above), LoSTIMs 1-4 will not bind the surface of the BMDDCs. These LoSTIMs carry the same PD-1 binding domain as LoSTIMs 5-8 and, therefore, they will still bind PD-1 on T cells.

OT-I cells co-cultured with BMDDC in the absence of antibody treatment were activated (63.6% proliferation by CFSE dilution and 42.6% CD69 expression). Treatment with LoSTIMs 1-4 did not inhibit T cell activation in these co-cultures. In fact, LoSTIMs 1-4 potentiated T cell activation to varying degrees. LoSTIM 1 had a minimal potentiating effect on T cell activation (68.4% proliferation and 56% CD69 expression). LoSTIM 2 markedly potentiated T cell activation (98.6% proliferation and 78.9% CD69 expression). LoSTIM 3 also potentiated T cell activation (90.1% proliferation and 61% CD69 expression). LoSTIM 4 also markedly potentiated T cell activation (96.1% proliferation and 79% CD69 expression).

These data demonstrate that binding to PD-1 on the T cell, by itself, is insufficient for a LoSTIM to mediate T cell inhibition. The LoSTIM binds to an antigen on the surface of an opposing cell in trans. Binding in cis to PD-1 and another antigen on the T cell surface is insufficient to mediate T cell inhibition, and this is demonstrated by the data in FIG. 7 and FIG. 4 because, in both cases, the OT-I T cells express both cognate binding antigens for the LoSTIMs used. Finally, the data in FIG. 7 demonstrate that a LoSTIM blocking the interaction of PD-1 with its natural ligands will potentiate, rather than inhibit, T cell activity if it does not also bind the surface of the opposing cell.

### Example 2: Representative Sequences of LoSTIMs Used in Example 1

TABLE 4A

LoSTIM 1 (SEQ ID NO: 1)
Ligand-ScFv Fusion
[ligand (bold font)]-[G4AG4 (italicized font)]-[ScFv VL (underlined font)]-[G4S x 3 (italicized font)]-[ScFv VH (underlined font)] in order of appearance
FTITAPKDLYVVEYGSNVTNECRFPVERELDLLALVVYWEKEDEQVIQFVAGEEDLKPQHSNFRGRA SLPKDQLLKGNAALQITDVKLQDAGVYCCIISYGGADYKRITLKVNAPYRKINQRISVDPATSEHEL ICQAEGYPEAEVIWTNSDHQPVSGKRSVTTSRTEGMLLNVTSSLRVNATANDVFYCTFWRSQPGQNH TAELIIPELPATHPPQNRTHGGGGAGGGGEVQVVETGGGLVQPGSSLKLSCESSGFTFSSTWMNWVR QAPGKGLEWVALVKDKYSNYEANYAESVKGRFIISRDDSKNRVYLQMNTLRDQDTATYHCTRSRGYK NWYFDFWGPGTMVTVSSGGGGSGGGGSGGGGSDIQMTQSPPSLSASLGDKVTITCQASQNINNYIAW YQQKPGKAPGLLILYTSTLVSGTPSRFSGSGSGRDYSFSISNVESEDIASYYCLQYDNSPRTFGGGT KLELK

LoSTIM 2
Ligand-IgG2a mAb Fusion
[ligand (bold font)]-[G4AG4 (italicized font)]-[VH (underlined font)]-[mIgG2a CH + Fc (regular font with mutations underlined and bolded)] in order of appearance

HC (SEQ ID NO: 2)
FTITAPKDLYVVEYGSNVTNECRFPVERELDLLALVVYWEKEDEQVIQFVAGEEDLKPQHSNFRGRA SLPKDQLLKGNAALQITDVKLQDAGVYCCIISYGGADYKRITLKVNAPYRKINQRISVDPATSEHEL ICQAEGYPEAEVIWTNSDHQPVSGKRSVTTSRTEGMLLNVTSSLRVNATANDVFYCTFWRSQPGQNH TAELIIPELPATHPPQNRTHGGGGAGGGGEVQVVETGGGLVQPGSSLKLSCESSGFTFSSTWMNWVR QAPGKGLEWVALVKDKYSNYEANYAESVKGRFIISRDDSKNRVYLQMNTLRDQDTATYHCTRSRGYK NWYFDFWGPGTMVTVSSAKTTAPSVYPLAPVCGDTTGSSVTLGCLVKGYFPEPVTLTWNSGSLSSGV HTFPAVLQSDLYTLSSSVTVTSSTWPSQSITCNVAHPASSTKVDKKIEPRGPTIKPCPPCKCPAPNL EGGPSVFIFPPKIKDVLMISLSPIVTCVVVDVSEDDPDVQISWFVNNVEVHTAQTQTHREDYNSTLR VVSALPIQHQDWMSGKAFACAVNNKDLPAPIERTISKPKGSVRAPQVYVLPPPEEEMTKKQVTLTCM VTDFMPEDIYVEWTNNGKTELNYKNTEPVLDSDGSYFMYSKLRVEKKNWVERNSYSCSVVHEGLHNH HTTKSFSRTPGK

LC (SEQ ID NO: 3) [VL (underlined font)]
DIQMTQSPPSLSASLGDKVTITCQASQNINNYIAWYQQKPGKAPGLLILYTSTLVSGTPSRFSGSGS GRDYSFSISNVESEDIASYYCLQYDNSPRTFGGGTKLELKRADAAPTVSIFPPSSEQLTSGGASVVC FLNNFYPKDINVKWKIDGSERQNGVLNSWTDQDSKDSTYSMSSTLTLTKDEYERHNSYTCEATHKTS TSPIVKSFNRNEC

LoSTIM 3
Bispecific IgG2a mAb with N-terminal scFv
[VL scFv (underlined font)]-[G4S x 3 (italicized font)]-[VH scFv (underlined font)]-[G4AG4 (italicized font)]-[VH (underlined font)]-[mIgG2a CH + Fc (regular font with mutations underlined and bolded)] in order of appearance
HC (SEQ ID NO: 4)
DVALTQTPVAQPVTLGDQASISCRSSQSLVHSNGRTYLEWYLQKPGQSPQLLIYKVSNRFSGVPDRF IGSGSGSDFTLTISRVEPEDLGVYYCFQATHDPNTFGAGTKLELKGGGGSGGGGSGGGGSQVQLQQS GAELVKPGSSVKISCKASGYTFTSHFIHWIKQQPGNGLEWIGGIYPGDGDTEYNQQFNGKATLTADK SSSTAYMRLSSLTSEDSAVYFCATRVPSYWFFDFWGPGTMVTVSSGGGGAGGGGEVQVVETGGGLVQ PGSSLKLSCESSGFTFSSTWMNWVRQAPGKGLEWVALVKDKYSNYEANYAESVKGRFIISRDDSKNR VYLQMNTLRDQDTATYHCTRSRGYKNWYFDFWGPGTMVTVSSAKTTAPSVYPLAPVCGDTTGSSVTL GCLVKGYFPEPVTLTWNSGSLSSGVHTFPAVLQSDLYTLSSSVTVTSSTWPSQSITCNVAHPASSTK VDKKIEPRGPTIKPCPPCKCPAPNLEGGPSVFIFPPKIKDVLMISLSPIVTCVVVDVSEDDPDVQIS WFVNNVEVHTAQTQTHREDYNSTLRVVSALPIQHQDWMSGKAFACAVNNKDLPAPIERTISKPKGSV RAPQVYVLPPPEEEMTKKQVTLTCMVTDFMPEDIYVEWTNNGKTELNYKNTEPVLDSDGSYFMYSKL RVEKKNWVERNSYSCSVVHEGLHNHHTTKSFSRTPGK

LC (SEQ ID NO: 5) [VL (underlined font)]
DIQMTQSPPSLSASLGDKVTITCQASQNINNYIAWYQQKPGKAPGLLILYTSTLVSGTPSRFSGSGS GRDYSFSISNVESEDIASYYCLQYDNSPRTFGGGTKLELKRADAAPTVSIFPPSSEQLTSGGASVVC FLNNFYPKDINVKWKIDGSERQNGVLNSWTDQDSKDSTYSMSSTLTLTKDEYERHNSYTCEATHKTS TSPIVKSFNRNEC

LoSTIM 4
Bispecific IgG2a (mut) mAb C-terminal scFv
[VH (underlined font)]-[mIgG2a CH + Fc (regular font with mutations underlined and bolded)]-[G4AG4 (italicized font)]-[VL scFv (underlined font)]-[G4S x 3 (italicized font)]-[VH scFv (underlined font)] in order of appearance

HC (SEQ ID NO: 6)
EVQVVETGGGLVQPGSSLKLSCESSGFTFSSTWMNWVRQAPGKGLEWVALVKDKYSNYEANYAESVK GRFIISRDDSKNRVYLQMNTLRDQDTATYHCTRSRGYKNWYFDFWGPGTMVTVSSAKTTAPSVYPLA PVCGDTTGSSVTLGCLVKGYFPEPVTLTWNSGSLSSGVHTFPAVLQSDLYTLSSSVTVTSSTWPSQS ITCNVAHPASSTKVDKKIEPRGPTIKPCPPCKCPAPNLEGGPSVFIFPPKIKDVLMISLSPIVTCVV VDVSEDDPDVQISWFVNNVEVHTAQTQTHREDYNSTLRVVSALPIQHQDWMSGKAFACAVNNKDLPA PIERTISKPKGSVRAPQVYVLPPPEEEMTKKQVTLTCMVTDFMPEDIYVEWTNNGKTELNYKNTEPV LDSDGSYFMYSKLRVEKKNWVERNSYSCSVVHEGLHNHHTTKSFSRTPGKGGGGAGGGGDVALTQTP VAQPVTLGDQASISCRSSQSLVHSNGRTYLEWYLQKPGQSPQLLIYKVSNRFSGVPDRFIGSGSGSD FTLTISRVEPEDLGVYYCFQATHDPNTFGAGTKLELKGGGGSGGGGSGGGGSQVQLQQSGAELVKPG SSVKISCKASGYTFTSHFIHWIKQQPGNGLEWIGGIYPGDGDTEYNQQFNGKATLTADKSSSTAYMR LSSLTSEDSAVYFCATRVPSYWFFDFWGPGTMVTVSS

LC (SEQ ID NO: 7) [VL (underlined font)]
DIQMTQSPPSLSASLGDKVTITCQASQNINNYIAWYQQKPGKAPGLLILYTSTLVSGTPSRFSGSGS GRDYSFSISNVESEDIASYYCLQYDNSPRTFGGGTKLELKRADAAPTVSIFPPSSEQLTSGGASVVC FLNNFYPKDINVKWKIDGSERQNGVLNSWTDQDSKDSTYSMSSTLTLTKDEYERHNSYTCEATHKTS TSPIVKSFNRNEC

LoSTIM 5 (SEQ ID NO: 8)
Ligand-ScFv Fusion
[ligand (bold font)]-[G4AG4 (italicized font)]-[ScFv VL (underlined font)]-[G4S x 3 (italicized font)]-[ScFv VH (underlined font)] in order of appearance FTITAPKDLYVVEYGSNVTNECRFPVERELDLLALVVYWEKEDEQVIQFVAGEEDLKPQHSNFRGRA SLPKDQLLKGNAALQITDVKLQDAGVYCCIISYGGADYKRITLKVNAPYRKINQRISVDPATSEHEL ICQAEGYPEAEVIWTNSDHQPVSGKRSVTTSRTEGMLLNVTSSLRVNATANDVFYCTFWRSQPGQNH TAELIIPELPATHPPQNRTHGGGGAGGGGDVQLVESGGGLVQPGGSRKLSCAASGFTFSSFGMHWVR QAPEKGLEWVAYISSGSNTIYYADTVKGRFTISRDNPKNTLFLQMTSLRSEDTAMYYCALTTGSWFA YWGQGTLVTVSAGGGGSGGGGSGGGGSDILLTQSPAILSVRPGERVSFSCRASQSLGTSIHWYQQRT DGSPRLLIKYASESISGIPSRFSGSGSGTDFTLSINSVESEDIADYYCQQSNSWPITFGAGTKLELK

LoSTIM 6
Ligand-mAb Fusion
[ligand (bold font)]-[G4AG4 (italicized font)]-[VH (underlined font)]-[CH + Fc (regular font with mutations underlined and bolded)] in order of appearance

HC (SEQ ID NO: 9)
FTITAPKDLYVVEYGSNVTNECRFPVERELDLLALVVYWEKEDEQVIQFVAGEEDLKPQHSNFRGRA SLPKDQLLKGNAALQITDVKLQDAGVYCCIISYGGADYKRITLKVNAPYRKINQRISVDPATSEHEL ICQAEGYPEAEVIWTNSDHQPVSGKRSVTTSRTEGMLLNVTSSLRVNATANDVFYCTFWRSQPGQNH TAELIIPELPATHPPQNRTHGGGGAGGGGDVQLVESGGGLVQPGGSRKLSCAASGFTFSSFGMHWVR QAPEKGLEWVAYISSGSNTIYYADTVKGRFTISRDNPKNTLFLQMTSLRSEDTAMYYCALTTGSWFA YWGQGTLVTVSAAKTTAPSVYPLAPVCGDTTGSSVTLGCLVKGYFPEPVTLTWNSGSLSSGVHTFPA VLQSDLYTLSSSVTVTSSTWPSQSITCNVAHPASSTKVDKKIEPRGPTIKPCPPCKCPAPNLEGGPS
VFIFPPKIKDVLMISLSPIVTCVVVDVSEDDPDVQISWFVNNVEVHTAQTQTHREDYNSTLRVVSAL PIQHQDWMSGKAFACAVNNKDLPAPIERTISKPKGSVRAPQVYVLPPPEEEMTKKQVTLTCMVTDFM PEDIYVEWTNNGKTELNYKNTEPVLDSDGSYFMYSKLRVEKKNWVERNSYSCSVVHEGLHNHHTTKS FSRTPGK

LC (SEQ ID NO: 10) [VL (underlined font)]
DILLTQSPAILSVRPGERVSFSCRASQSLGTSIHWYQQRTDGSPRLLIKYASESISGIPSRFSGSGS GTDFTLSINSVESEDIADYYCQQSNSWPITFGAGTKLELKRADAAPTVSIFPPSSEQLTSGGASVVC FLNNFYPKDINVKWKIDGSERQNGVLNSWTDQDSKDSTYSMSSTLTLTKDEYERHNSYTCEATHKTS TSPIVKSFNRNEC

LoSTIM 7
Bispecific IgG2a (mut) mAb with N-terminal scFv
[VL scFv (underlined font)]-[G4S x 3 (italicized font)]-[VH scFv (underlined font)]-[G4AG4 (italicized font)]-[VH]-[CH + Fc (regular font with mutations underlined and bolded)] in order of appearance

HC (SEQ ID NO: 11)
DVALTQTPVAQPVTLGDQASISCRSSQSLVHSNGRTYLEWYLQKPGQSPQLLIYKVSNRFSGVPDRF IGSGSGSDFTLTISRVEPEDLGVYYCFQATHDPNTFGAGTKLELKGGGGSGGGGSGGGGSQVQLQQS GAELVKPGSSVKISCKASGYTFTSHFIHWIKQQPGNGLEWIGGIYPGDGDTEYNQQFNGKATLTADK SSSTAYMRLSSLTSEDSAVYFCATRVPSYWFFDFWGPGTMVTVSSGGGGAGGGGDVQLVESGGGLVQ PGGSRKLSCAASGFTFSSFGMHWVRQAPEKGLEWVAYISSGSNTIYYADTVKGRFTISRDNPKNTLF LQMTSLRSEDTAMYYCALTTGSWFAYWGQGTLVTVSAAKTTAPSVYPLAPVCGDTTGSSVTLGCLVK GYFPEPVTLTWNSGSLSSGVHTFPAVLQSDLYTLSSSVTVTSSTWPSQSITCNVAHPASSTKVDKKI EPRGPTIKPCPPCKCPAPNLEGGPSVFIFPPKIKDVLMISLSPIVTCVVVDVSEDDPDVQISWFVNN VEVHTAQTQTHREDYNSTLRVVSALPIQHQDWMSGKAFACAVNNKDLPAPIERTISKPKGSVRAPQV YVLPPPEEEMTKKQVTLTCMVTDFMPEDIYVEWTNNGKTELNYKNTEPVLDSDGSYFMYSKLRVEKK NWVERNSYSCSVVHEGLHNHHTTKSFSRTPGK

LC (SEQ ID NO: 12) [VL (underlined font)]
DILLTQSPAILSVRPGERVSFSCRASQSLGTSIHWYQQRTDGSPRLLIKYASESISGIPSRFSGSGS GTDFTLSINSVESEDIADYYCQQSNSWPITFGAGTKLELKRADAAPTVSIFPPSSEQLTSGGASVVC FLNNFYPKDINVKWKIDGSERQNGVLNSWTDQDSKDSTYSMSSTLTLTKDEYERHNSYTCEATHKTS TSPIVKSFNRNEC

LoSTIM 8
Bispecific IgG2a (mut) mAb C-terminal scFv
[VH (underlined font)]-[CH + Fc (regular font with mutations underlined and bolded)]-[G4AG4 (italicized font)]-[VL scFv (underlined font)]-[G4S x 3 (italicized font)]-[VH scFv (underlined font)] in order of appearance

HC (SEQ ID NO: 13)
DVQLVESGGGLVQPGGSRKLSCAASGFTFSSFGMHWVRQAPEKGLEWVAYISSGSNTIYYADTVKGR FTISRDNPKNTLFLQMTSLRSEDTAMYYCALTTGSWFAYWGQGTLVTVSAAKTTAPSVYPLAPVCGD TTGSSVTLGCLVKGYFPEPVTLTWNSGSLSSGVHTFPAVLQSDLYTLSSSVTVTSSTWPSQSITCNV AHPASSTKVDKKIEPRGPTIKPCPPCKCPAPNLEGGPSVFIFPPKIKDVLMISLSPIVTCVVVDVSE DDPDVQISWFVNNVEVHTAQTQTHREDYNSTLRVVSALPIQHQDWMSGKAFACAVNNKDLPAPIERT ISKPKGSVRAPQVYVLPPPEEEMTKKQVTLTCMVTDFMPEDIYVEWTNNGKTELNYKNTEPVLDSDG SYFMYSKLRVEKKNWVERNSYSCSVVHEGLHNHHTTKSFSRTPGKGGGGAGGGGDVALTQTPVAQPV TLGDQASISCRSSQSLVHSNGRTYLEWYLQKPGQSPQLLIYKVSNRFSGVPDRFIGSGSGSDFTLTI SRVEPEDLGVYYCFQATHDPNTFGAGTKLELKGGGGSGGGGSGGGGSQVQLQQSGAELVKPGSSVKI SCKASGYTFTSHFIHWIKQQPGNGLEWIGGIYPGDGDTEYNQQFNGKATLTADKSSSTAYMRLSSLT SEDSAVYFCATRVPSYWFFDFWGPGTMVTVSS

LC (SEQ ID NO: 14) [VL (underlined font)]
DILLTQSPAILSVRPGERVSFSCRASQSLGTSIHWYQQRTDGSPRLLIKYASESISGIPSRFSGSGS GTDFTLSINSVESEDIADYYCQQSNSWPITFGAGTKLELKRADAAPTVSIFPPSSEQLTSGGASVVC
FLNNFYPKDINVKWKIDGSERQNGVLNSWTDQDSKDSTYSMSSTLTLTKDEYERHNSYTCEATHKTS TSPIVKSFNRNEC

### Example 3: Various Representative Uses of LoSTIMs

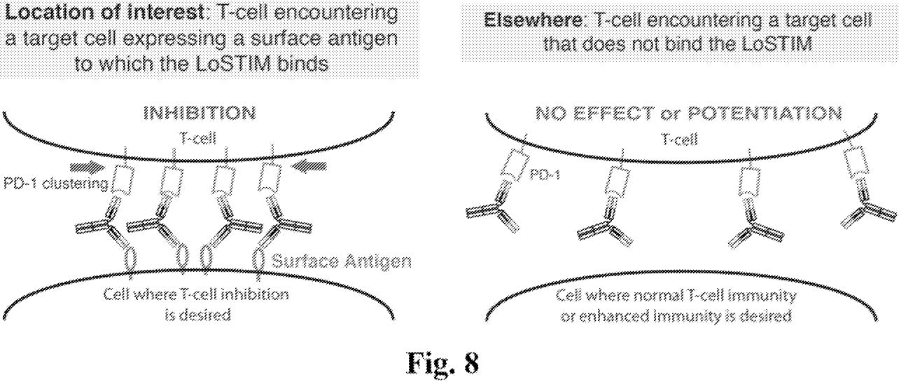

A conceptual overview of LoSTIMs is shown in FIG. 8. The trans bispecific binding of a LoSTIM to PD-1 on the T cell and to a cell-surface antigen on the opposing target cell results in PD-1 clustering on the T cell surface and co-localization of this cluster with the immunological synapse, thereby activating PD-1 and inhibiting T cell activity. PD-1 is free to move laterally on the T cell surface and it is activated when clustered in proximity to the immunological synapse. Thus, trans bispecific binding of the LoSTIM to the surface antigen on the target cell and to PD-1 on the T cell will recruit and cluster PD-1 at the cell-cell interface, where the immunological synapse is located. This activates PD-1, resulting in inhibition of TCR signaling and T cell activity. Engagement of the antigen on the target cell surface is necessary for a LoSTIM to function as a PD-1 agonist: simply binding PD-1 alone will not cluster and co-localize PD-1 to the immunological synapse and will therefore not inhibit T cell activity. This property allows a LoSTIM to function as selective PD-1 agonist, inhibiting T cell activity only in tissues that express the surface antigen. A LoSTIM may or may not be designed to block the engagement of PD-1 by its natural ligands. If it is designed to block the engagement of PD-1 by its ligands, it will act as an antagonist of PD-1 on T cells interacting with tissues that do not express the surface antigen, while still acting as an agonist of PD-1 on T cells interacting with tissues that do express the surface antigen. A single LoSTIM molecule of this design will therefore function as either an inhibitor or potentiator of T cell activity depending on the tissue context.

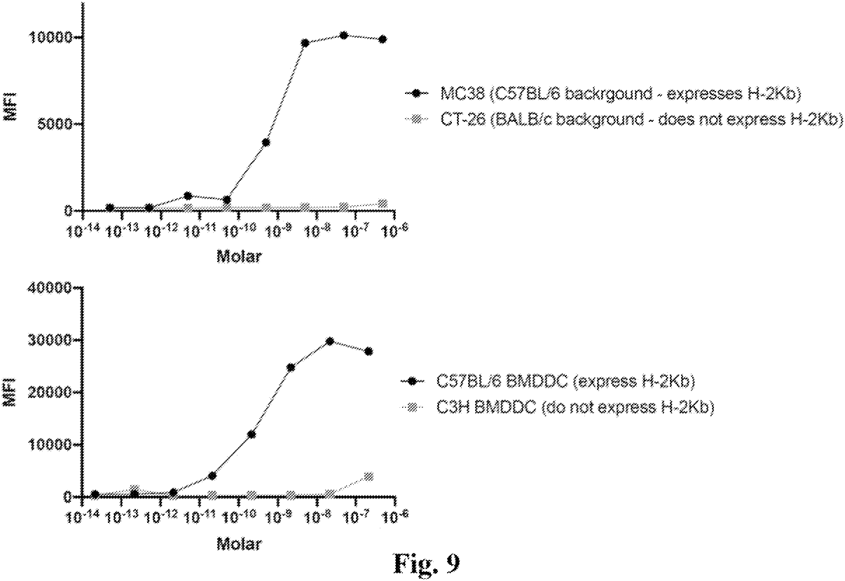

Binding to cells that express the model surface antigen H-2K^{b} is shown in FIG. 9. Binding to H-2K^{b} was assessed by flow cytometry using the C57BL/6 colorectal cancer cell line MC38, which is known to express H-2K^{b}. Specificity was demonstrated by assessing binding to CT-26 cells, known not to express H-2Kb. The LoSTIM bound avidly to MC38 cells but not to CT-26 cells. In a separate experiment, binding to H-2K^{b} was assessed by flow cytometry using BMDDC from C57BL/6 mice, which express H-2K^{b}, or BMDDC from C3H mice, which do not express H-2K^{b}. The LoSTIM bound avidly to C57BL/6 BMDDC cells but not to C3H BMDDC cells.

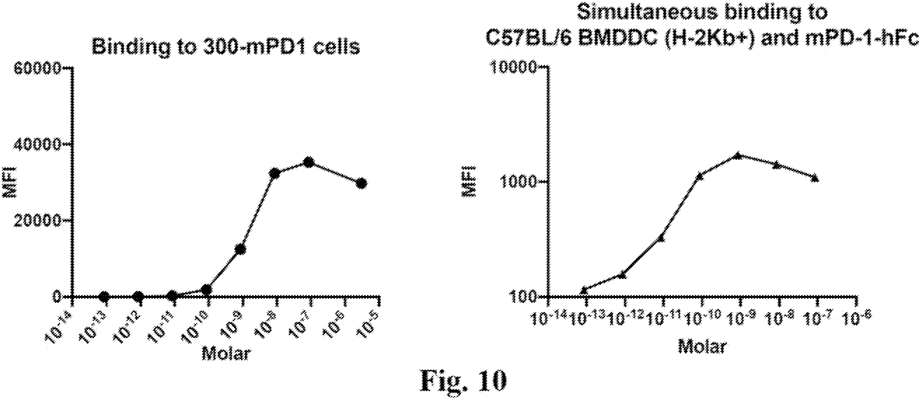

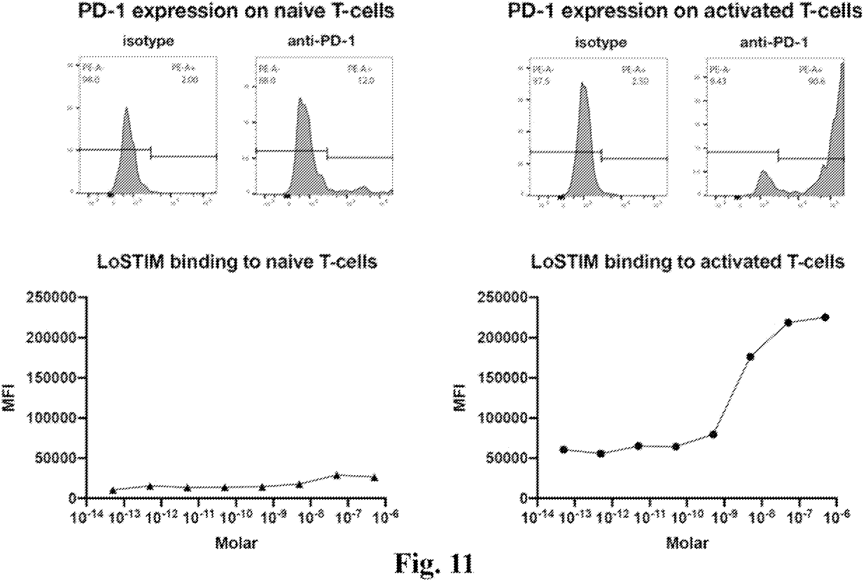

Binding to PD-1 and bispecific binding to PD-1 and H-2K^{b} is shown in FIG. 10. Binding of the LoSTIM to PD-1 was assessed by flow cytometry using 300-mPD-1 cells, transgenic mouse pro-B cells that stably express mouse PD-1. The LoSTIM bound 300-mPD-1 cells avidly. Simultaneous bispecific binding to PD-1 and H-2K^{b} was assessed by flow cytometry using MC38 cells, which express H-2K^{b} but not PD-1, and a soluble mouse PD-1-Fc fusion bearing a human IgG1 Fc domain (mPD-1-hFc). Binding of the mPD-1-hFc to MC38 cells as measured by a fluorophore-labeled an anti-human IgG secondary antibody indicated bispecific binding of the LoSTIM to H-2K^{b} on MC38 cells and to the mPD-1-hFc. This also confirmed the specificity of PD-1 binding.

Futher experiments, as shown in FIG. 11, desmonstrated that LoSTIM binds to T cells that express PD-1 but not to T cells that do not express PD-1. To further confirm PD-1 binding specificity, binding of the LoSTIM to activated primary T cells that express PD-1 was compared with binding to naive primary T cells that do not express PD-1. These T cells were obtained from mice from BALB/cByJ mice that do not express H-2K^{b}, therefore binding to activated T cells should only occur if the LoSTIM binds specifically to PD-1. The LoSTIM bound avidly to activated BALB/cByJ T cells but not to naive BALB/cByJ T cells, further confirming the specificity of PD-1 binding.

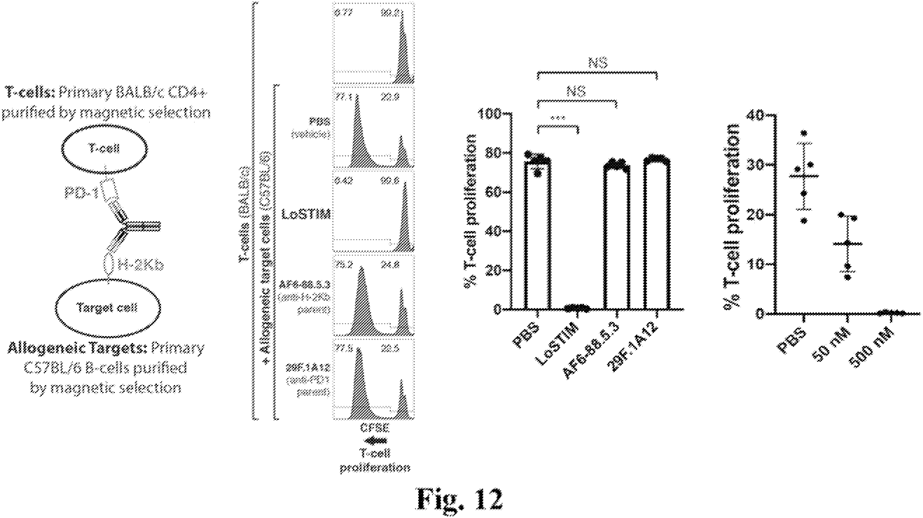

Inhibition of T cell activation was investigated as shown in FIG. 12. To assess the effect of the LoSTIM on T cell activation by APCs that express the H-2K^{b} model surface antigen, primary CD4+ BALB/cByJ T cells (H-2K^{b} negative) were co-cultured with B-cells obtained from C57BL/6 mice (H-2K^{b} positive) at a 1:1 ratio for five days. Cultures were treated with 500 nanomolar of either LoSTIM, the parent anti-H-2K^{b} antibody (AF6-88.5.3), the parent anti-PD-1 antibody (29F.1A12), or vehicle (PBS). The LoSTIM completely inhibited APC-induced proliferation of T cells as measured by CFSE dilution. Neither AF6-88.5.3, 29F.1A12, nor vehicle affected T cell proliferation. This demonstrates that the LoSTIM inhibits T cell activation by APCs that express a surface antigen that binds the LoSTIM. The bispecific binding of the LoSTIM to PD-1 on the T cell and to the surface antigen on the target cell was necessary for this effect, as demonstrated by a lack of T cell inhibition by either AF6-88.5.3, which binds the model surface antigen H-2K^{b}, or 29F.1A12, which binds PD-1. In a similar separate experiment, two concentrations of LoSTIM were used and the inhibition of T cell proliferation was found to be dose-dependent.

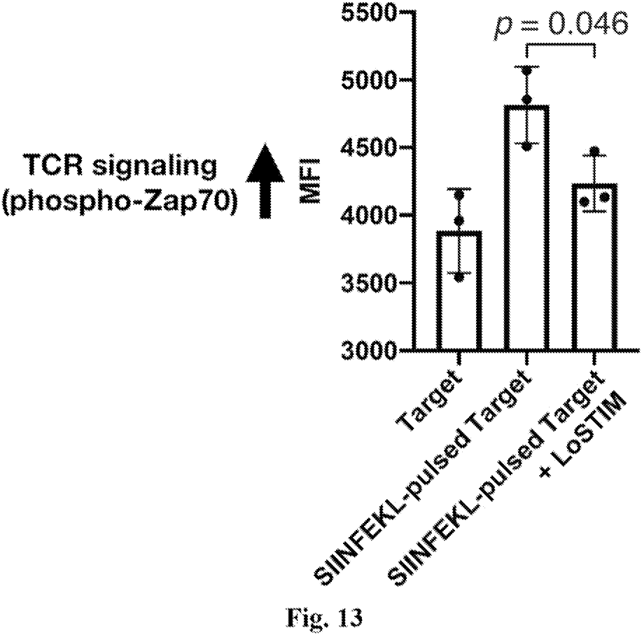

Inhibition of TCR signaling was investigated as shown in FIG. 13. To assess the effect on TCR signaling, CD8+ T cells expressing a transgenic TCR that recognizes the ovalbumin antigen SIINFEKL were purified from OT-1 mice, activated by plate-bound anti-CD3/CD28 antibody, rested, and then re-stimulated by C57BL/6 cells loaded with the SIINFEKL antigen. TCR activation was assessed by phosphoflow cytometry using an a fluorophore-labeled antibody specific for phosphorylated Zap70. Zap70 is a membrane-proximal kinase that is phosphorylated and activated upon TCR activation and is responsible for TCR signal transduction. PD-1 activation is known to inhibit TCR-mediated Zap70 phosphorylation. Treatment with LoSTIM inhibited antigen-induced Zap70 phosphorylation.

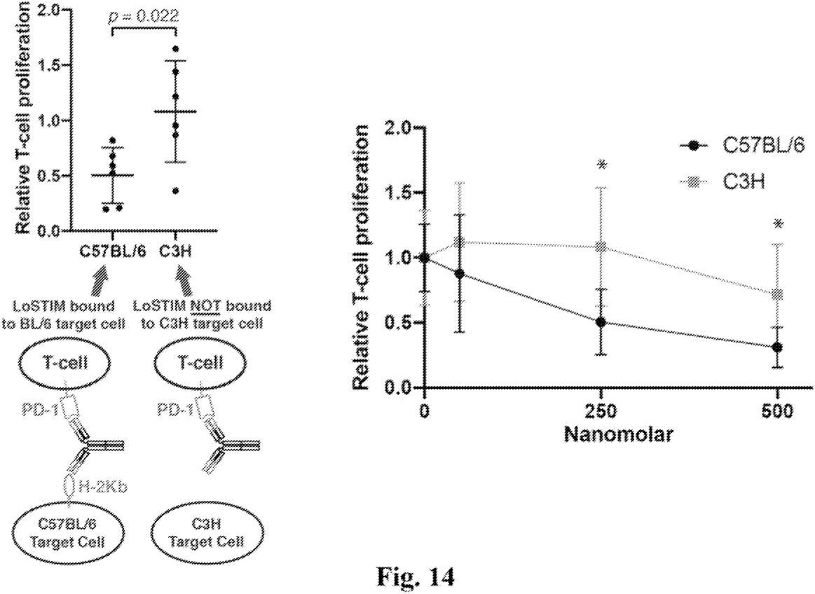

Cell-specific and/or tissue-specific inhibition of T cell activation was investigated as shown in FIG. 14. The effect of the LoSTIM was compared when T cells were co-cultured with APCs that express the model cell surface antigen H-2K^{b} and when T cells were co-cultured with APCs that do not express H-2K^{b}. To accomplish this, purified BALB/cByJ CD8+ T cells were co-cultured with either BMDDC from C57BL/6 mice, which express the model cell surface antigen H-2K^{b}, or BMDDC from C3H mice, which do not express H-2K^{b}. Cultures were treated with varying concentrations of LoSTIM. Relative T cell proliferation was calculated by normalizing the percentage T cell proliferation by the percentage T cell proliferation of co-cultures treated with vehicle (PBS). Because the strength of allostimulation can vary between backgrounds, this calculation was performed separately for BALB/cByJ T cells stimulated by C57BL/6 BMDDC and C3H BMDDC. T cell activation was inhibited when the LoSTIM bound the target cell but did was not inhibited when the LoSTIM did not bind the target cell.

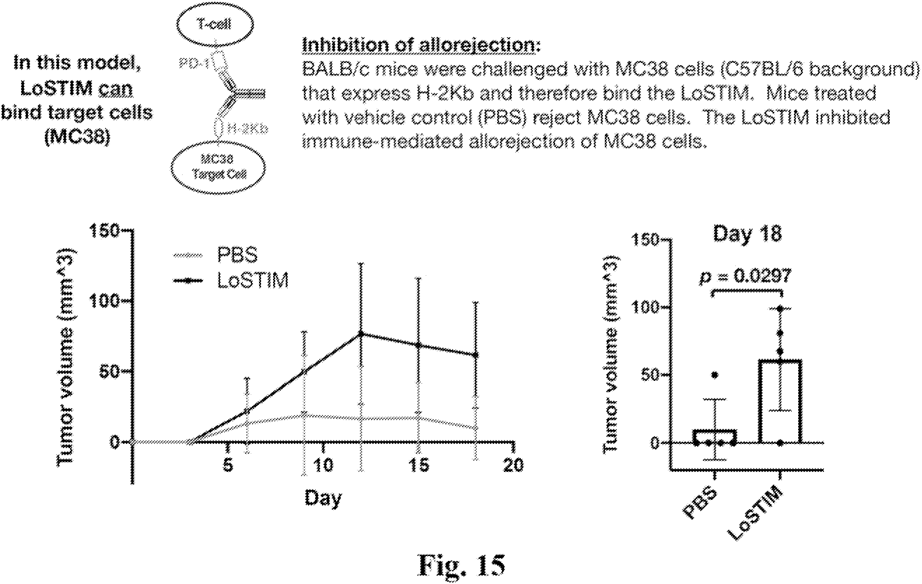

Inhibition of allorejection in vivo was investigated as shown in FIG. 15. A model of allorejection was used to assess the ability of the LoSTIM to inhibit immune activity in vivo against target cells that express an antigen that binds the LoSTIM. MC38 cells (of C57BL/6 origin) were injected subcutaneously in BALB/cByJ mice. Tumor volumes were significantly larger in mice that received intraperitoneal injections of LoSTIM when compared with mice that received PBS as a vehicle control (p = 0.0297). This demonstrates that the LoSTIM can inhibit immune activity against target cells in vivo, if the target cells express an antigen to which the LoSTIM binds.

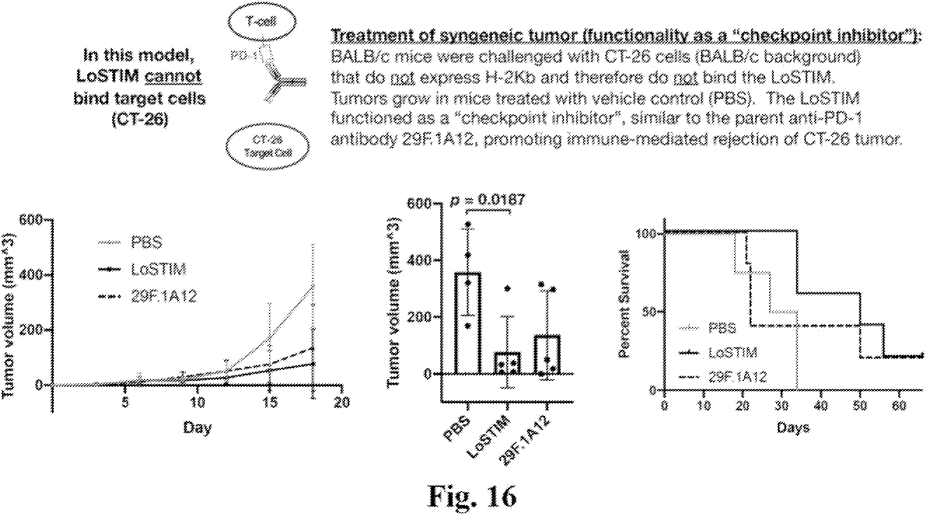

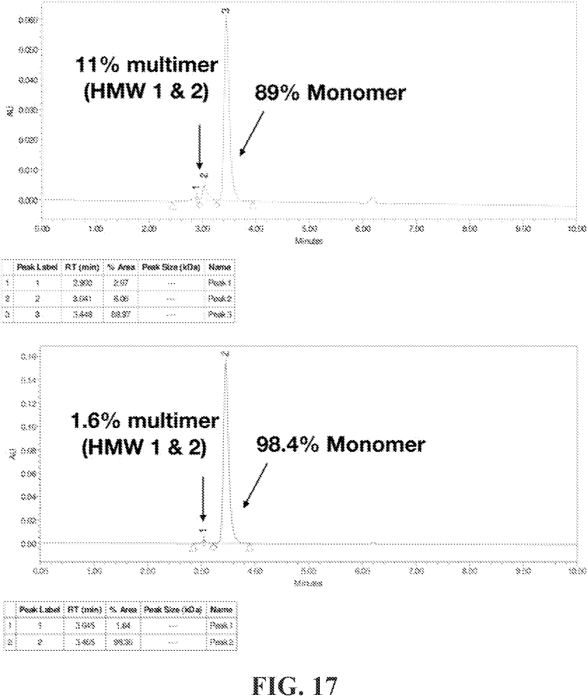

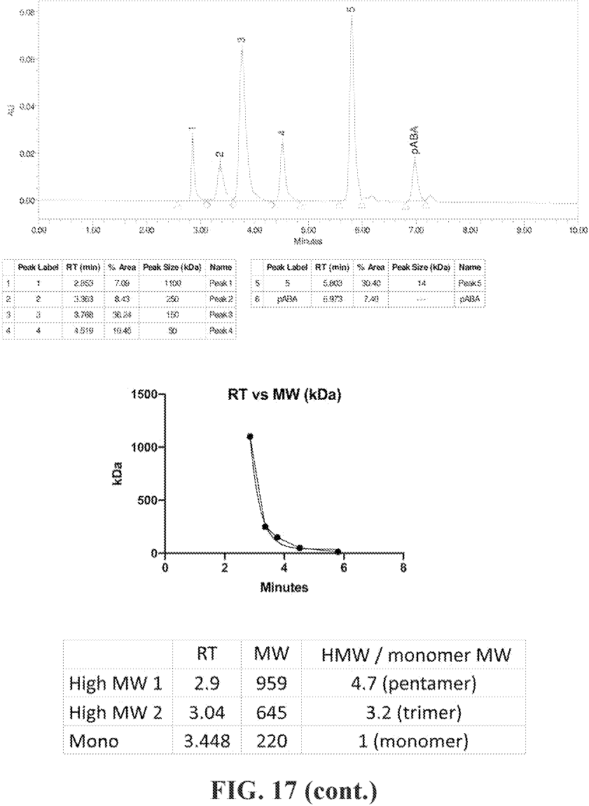

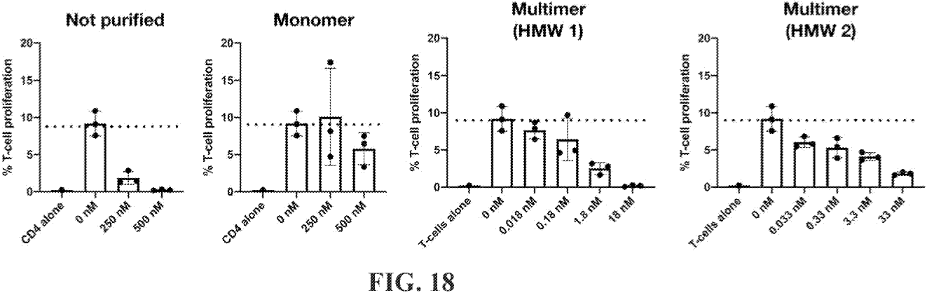

Treatment of syngenic tumor in vivo was investigated as shown in FIG. 16. A model of syngeneic tumor treatment was used to assess the in vivo effect of the LoSTIM on immune activity against target cells that do not express an antigen that binds the LoSTIM. CT-26 cells were injected subcutaneously in BALB/cByJ mice and mice were treated with either LoSTIM, 29F.1A12 (the parent anti-PD-1 antibody used in design of the LoSTIM), or PBS as a vehicle control. The LoSTIM did not inhibit immune activity against CT-26 cells. Instead, the LoSTIM promoted immune activity against CT-26 cells, functioning similarly to the PD-1 inhibitor 29F.1A12. This demonstrates that the LoSTIM will not inhibit immune activity against target cells that do not express an antigen to which it binds. Moreover, it demonstrates that a LoSTIM that blocks the natural ligation of PD-1 will function as a PD-1 inhibitor in these contexts.

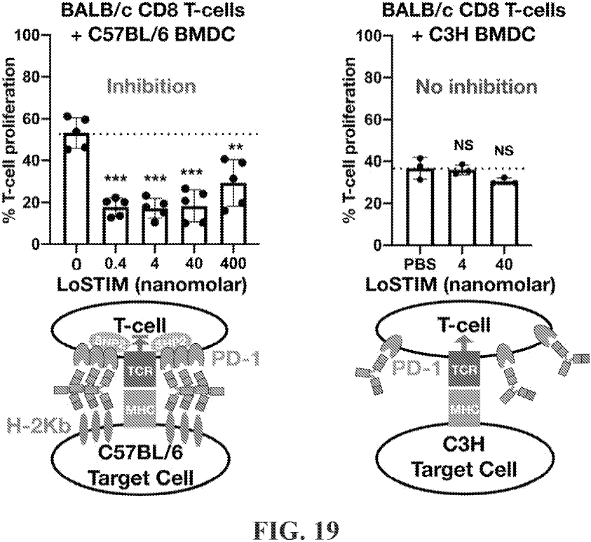

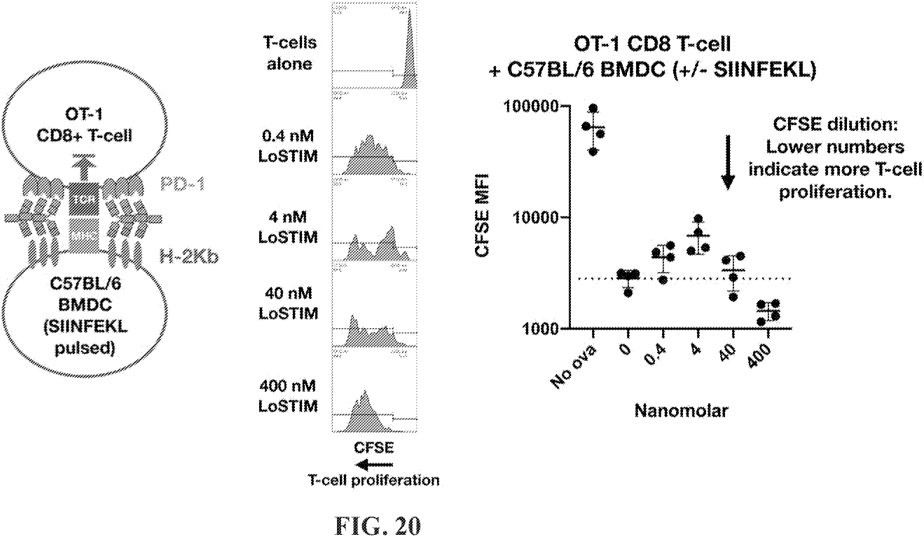

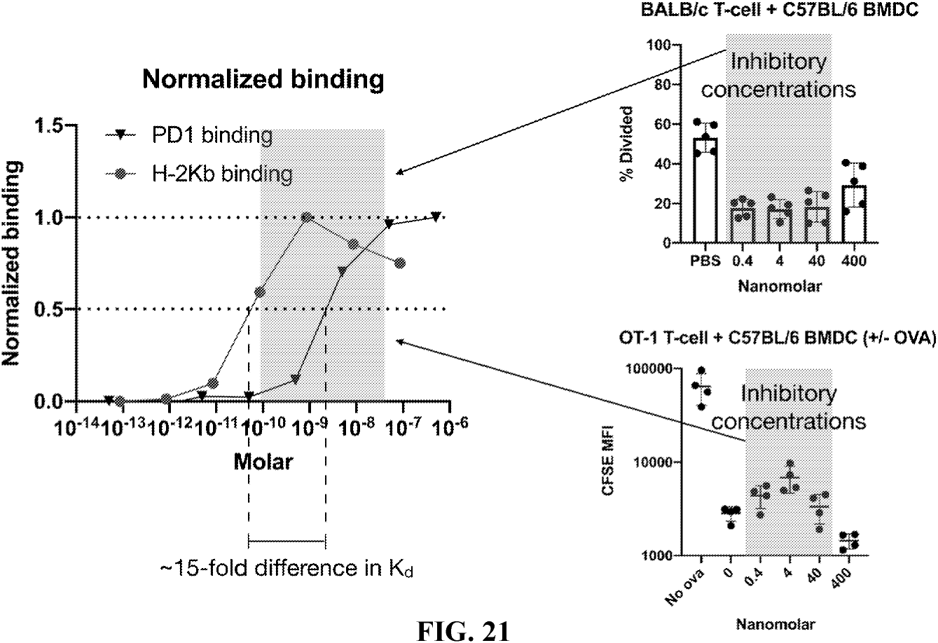

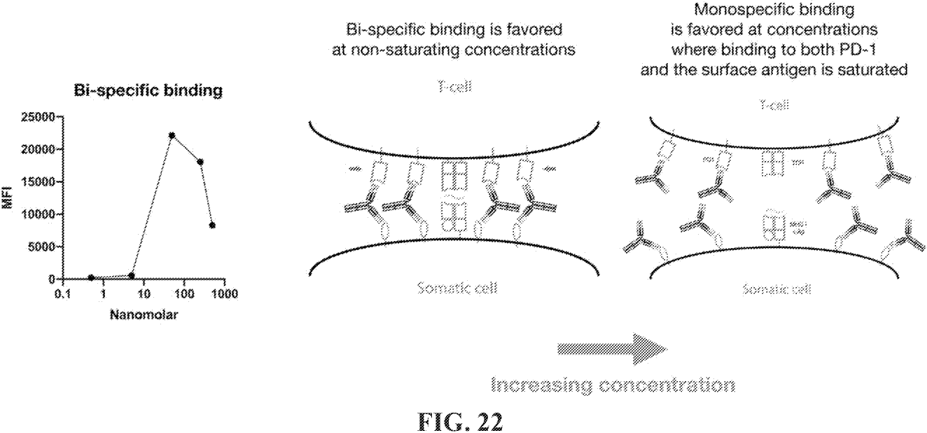

Moreover, experiments were performed demonstrating that T-cell inhibition occurs when the LoSTIM is present at a concentration where binding to both of its binding partners (PD-1 and the tissue antigen) is not saturated (FIGS. 19-21), thereby confirming that the LoSTIM functions as a PD-1 agonist below the concentration where the binding curves for both PD-1 and the tissue antigen predict maximal binding (FIG. 21). Without being bound by theory, it is believed that a single LoSTIM molecule binds simultaneously to the tissue antigen and PD-1 on the opposing T-cell surface and this is favored at non-saturating concentrations. Conversely, at concentrations where the binding to each binding partner is maximal (saturating concentrations), mono-specific binding to each binding partner is favored, which does not result in PD-1 clustering. For example, FIG. 21 shows that true bispecific binding increases as concentration increases before decreasing as the concentration approaches the saturating concentration of the lowest affinity binding partner. The net result is that LoSTIMs with a wider differential between affinities for their two binding partners are believed to possess a broader range of concentrations where they function as PD-1 agonists and thus have a wider therapeutic window. Conversely, PD-1 bispecifics that have a more similar binding affinity for their two binding partners would have a narrower range of concentrations where they exhibit true bispecific binding facilitating PD-1 agonism. This results explains why anti-PD-1/anti-PD-L1 bispecific antibodies can function as both PD-1 and PD-L1 inhibitors (dual checkpoint inhibitors) while not resulting in appreciable PD-1 clustering and agonism since such antibodies are believed to have similar affinity for each of their binding partners, and because of this, there would be little appreciable window of concentrations where they can cluster PD-1. This applies to both blocking and non-blocking LoSTIMs.

Generally, a LoSTIM that is intended to function as a PD-1 inhibitor systemically, while also functioning as a PD-1 agonist locally (e.g., for cancer treatment) should benefit from having a higher affinity for PD-1 than for the tissue-specific surface antigen. As discussed above, its binding to PD-1 ideally should be at or near saturation to ensure blockade of PD-L1 at the tumor site, but binding to both PD-1 and the surface antigen should not both be saturated in order for the LoSTIM to function as a PD-1 agonist on surface antigen-expressing tissue.

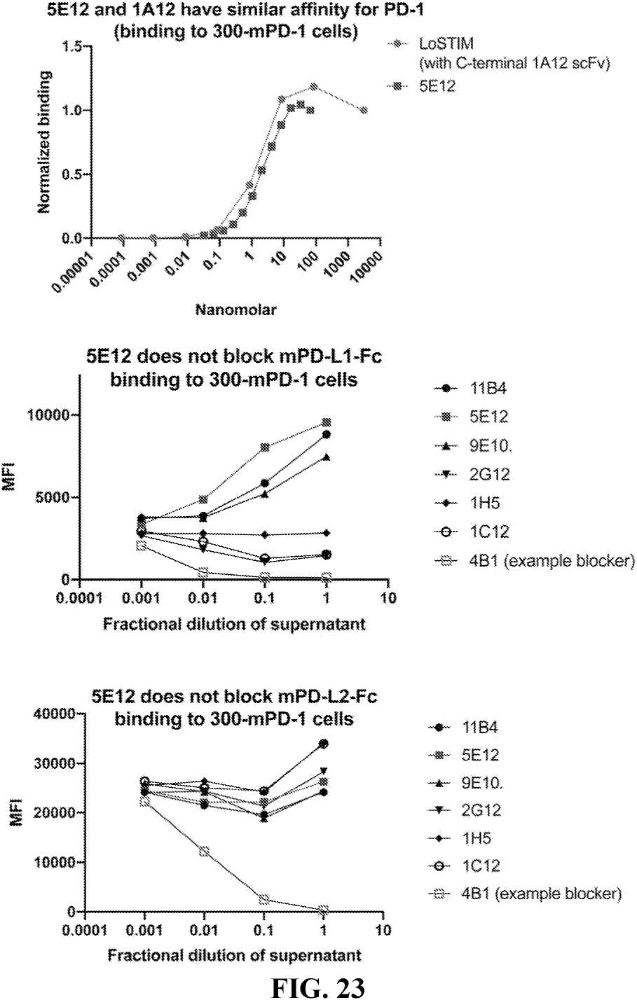

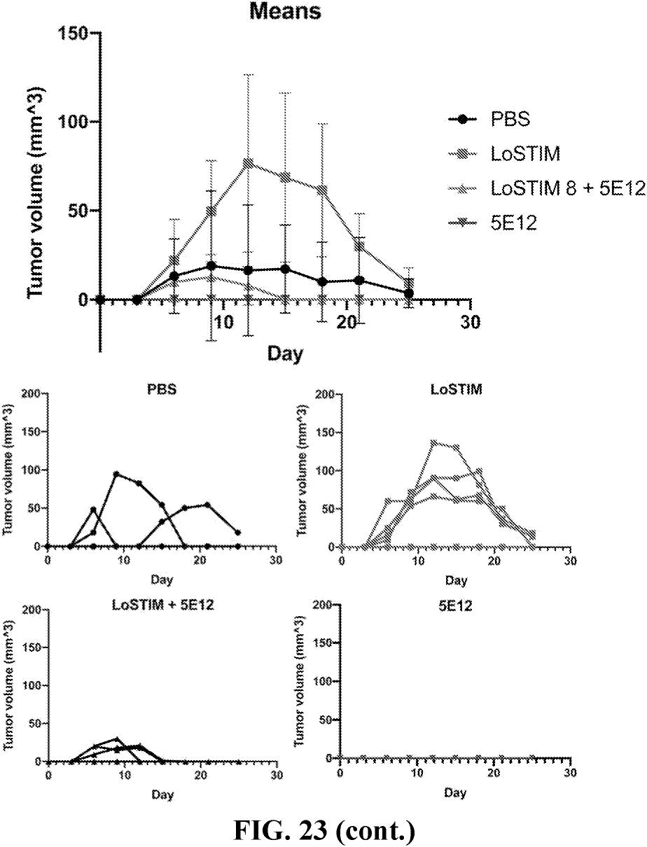

In some embodiments, additional LoSTIM design characteristics may be useful and are contemplated. For example, in a LoSTIM that has a higher affinity for PD-1, it is believed to be advantageous, in some embodiments, for it to be a monovalent bispecific (have one binding site rather than two binding sites for each binding partner, especially for PD-1). Without being bound by theory, it is believed that, when in solution at a concentration that is saturating for PD-1 binding, a bivalent LoSTIM could bind more than one copy of PD-1 on the T-cell surface at the same time, and, in doing so, bridge these two copies of PD-1 at a distance or an orientation that is incompatible with their activation. The data described herein indicate that a bivalent monospecific ant-PD-1 antibody (e.g., mAb 5E12) can bridge PD-1 at a distance or orientation that is incompatible with its activation (FIG. 23). mAb 5E12 is an anti-PD-1 antibody that does not block the ligation of PD-1 by PD-L1, so it would not be expected to function as a checkpoint inhibitor. Yet, it appears to still promote the clearance of tumor (e.g., a tumor allograft is rejected far more quickly in 5E12 treated mice than in mice treated with vehicle). Since mAb 5E12 binds PD-1 but does not block PD-L1 binding, the data provided herein indicate that it disrupts PD-1 function using a different mechanism, which is believed to be that stabilized PD-1 in a dimer is in some way incompatible with activation and which determination that LoSTIMs as a bispecific molecules having a single PD-1 binding site advantage is unexpected. Any anti-PD-1 clone should be able to be used in a monovalent bispecific LoSTIM provided it has a difference in binding affinities for PD-1 and the surface antigen and/or ii) has a higher binding affinity for PD-1 than the surface antigen in LoSTIMs that act as checkpoint inhibitors systemically while also functioning as a PD-1 agonist locally, as described above.

In some embodiments, a LoSTIM may have an engineered Fc domain, such as to attenuate binding of the Fc to an IgG antibody receptor like an Fc-gamma receptor (e.g., FcyRI, FcyRIIA, FcyRIIB 1, FcyRIIB2, FcyRIIIA, and/or FcyRIIIB). This is useful in some embodiments because Fc-gamma receptor binding may cause the LoSTIM to bind the surface of professional antigen presenting cells that express Fc-gamma receptors, thus inhibiting or partially inhibiting the priming of new T-cell responses.

As described herein, surface antigen choice for LoSTIM targeting is not particularly limited as long as there is expression of the surface antigen at any level on the desired tissue for protection and not on the cancer or other tissue of interest. However, in some embodiments, certain surface antigens may be particularly useful for certain clinical applications. For example, as described herein, for LoSTIMs capable of treating solid malignancy while also treating or prophylaxing against immune related adverse events (irAE), the surface antigen must be expressed on the tissue where immunotolerance is desired (tissue affected by or at risk of being affected by an irAE) but should not be expressed on cancer cells. Since the antigens expressed by different cancers and different peripheral tissues may not be the same, different antigens are appropriate to protect certain tissues at risk of irAE in the context of treatment for a given cancer type of interest. Some surface antigens are appropriate for LoSTIMs are useful across multiple clinical scenarios. Thus, in addition to biophysical criteria for compatibility of a PD-1 binding moiety and a surface antigen moiety in a LoSTIM, certain criteria for selecting particular surface antigens in the context of a clinical application may include one or more of the following: 1) presence on the plasma membrane of the cell, 2) presence on the basolateral surface of the plasma membrane in cells that comprise epithelial tissue, and 3) surface antigen expression level and/or surface density on the portion of the cell surface that may come in contact with immune cells (e.g., the basolateral surface of cells comprising epithelial tissue).

Well-known bioinformatics analyses can be applied to analyzing publicly available and well-known gene expression and protein expression data sources in order to identify surface antigens of interest. For example, the Human Protein Atlas dataset (version 20.1) was downloaded from the World Wide Web at proteinatlas.org/about/download. This dataset comprises the protein expression data from 44 normal human tissue types derived from antibody-based protein profiling using immunohistochemistry. Protein expression profiles from 17 forms of human cancer were also downloaded from this source (version 20.1). A list of membrane proteins (membrane-integral and membrane-associated) was downloaded from The Universal Protein Resource (Uniprot) available on the World Wide Web at uniprot.org (the following search terms were used: locations:(location:“Membrane [SL-0162]”) AND reviewed:yes AND organism:“Homo sapiens (Human) [9606]”). A set of custom computer programs written in the Matlab computer language were then used to integrate these datasets into a single database in which normal tissue expression profiles and cancer tissue expression profiles are linked for each protein in the database. After filtering the database to remove any proteins not listed as a membrane protein in the list obtained from Uniprot, the database was searched for proteins that meet criteria appropriate for each clinical application. For applications involving the treatment of cancer, such as treatment of irAE or GVHD, a surface antigen was appropriate if it was expressed on the tissue where immunotolerance is desired but not expressed on the cancer being treated. Representative, non-limiting examples of surface antigens for advantageous use in certain clinical applications include the following table: Table 3

TABLE 3A

List of proteins that meet criteria and have at least low expression on tissues where immune cell inhibition is desired (Ensembl version 92.38 ID for is provided for each gene symbol):
List of proteins that meet criteria and have at least LOW expression on tissues where immune cell inhibition is desired (Ensembl version 92.38 ID for is provided for each gene symbol):
TTYH3(ENSG00000136295), AXL(ENSG00000167601), VPS52(ENSG00000223501), MS4A8(ENSG00000166959), MAP1LC3A(ENSG00000101460), CD36(ENSG00000135218), FCAMR(ENSG00000162897), MGAT4C(ENSG00000182050), SCGN(ENSG00000079689), SLC9A2(ENSG00000115616), TM4SF18(ENSG00000163762), SNTG2(ENSG00000172554), PCSKS(ENSG00000099139), ALPL(ENSG00000162551), SELENBPl(ENSG00000143416), STX1A(ENSG00000106089), SNX24(ENSG00000064652), SMIM24(ENSG00000095932), SMURF1(ENSG00000198742), NAAA(ENSG00000138744), SLC27A2(ENSG00000140284), SEC13(ENSG00000157020), SLC41A2(ENSG00000136052), SOCS7(ENSG00000274211), QRFPR(ENSG00000186867), RPH3AL(ENSG00000181031), MPP7(ENSG00000150054), RAC1(ENSG00000136238), RNF145(ENSG00000145860), SERINC3(ENSG00000132824), ARHGAP5(ENSG00000100852), NFAM1(ENSG0000023SS68), HCRTR1(ENSG00000121764), LDLR(ENSG00000130164), GPR65(ENSG00000140030), RAC3(ENSG00000169750), MUC12(ENSG00000205277), WSCD1(ENSG00000179314), HTRAl(ENSG00000166033), RAB27B(ENSG00000041353), KLRG1(ENSG00000139187), PARVB(ENSG00000188677), DLC1(ENSG00000164741), PLCB3(ENSG00000149782), PCDHA4(ENSG00000204967), RECK(ENSG00000122707), MUC1(ENSG00000185499), PSTPIP2(ENSG00000152229), RAB3B(ENSG00000169213), PSCA(ENSG00000167653), KCNH8(ENSG00000183960), SDC1(ENSG00000115884), TMEM45A(ENSG00000181458), TPSG1(ENSG00000116176), TRPM7(ENSG00000092439), SLC18A1(ENSG00000036565), SPPL3(ENSG00000157837), STYK1(ENSG00000060140), TMEM134(ENSG00000172663), MAPKAP1(ENSG00000119487), IFI27(ENSG00000165949), ITLN1(ENSG00000179914), PTGDR(ENSG00000168229), SGMS1(ENSG00000198964), SYNGR4(ENSG00000105467), ITGB6(ENSG00000115221), MUC15(ENSG00000169550), PEX10(ENSG00000157911), OSBPL7(ENSG00000006025), CD46(ENSG00000117335), PROM2(ENSG00000155066), PLPP7(ENSG00000160539), MGAT4A(ENSG00000071073), LRRN2(ENSG00000170382), OBSCN(ENSG00000154358), PROM1(ENSG00000007062), UGT2B4(ENSG00000156096), TMEM72(ENSG00000187783), TMEM132A(ENSG00000006118), VSIG10(ENSG00000176834), SYT11(ENSG00000132718), TMEM236(ENSG00000148483), SLC7A7(ENSG00000155465), TMEM143(ENSG00000161558), VPS35L(ENSG00000103544), ZP2(ENSG00000103310), SYTL4(ENSG00000102362), SYTL3(ENSG00000164674), TMEM41A(ENSG00000163900), TSPAN8(ENSG00000127324), UNC13B(ENSG00000198722), PLAUR(ENSG00000011422), SYTL2(ENSG00000137501), PRRG2(ENSG00000126460), TPRA1(ENSG00000163870), SLC46A2(ENSG00000119457), OCLN(ENSG00000197822), RNF170(ENSG00000120925), SPHK1(ENSG00000176170), SLC28A3(ENSG00000197506), SCAMPS(ENSG00000198794), PTAFR(ENSG00000169403), SYP(ENSG00000102003), PIGZ(ENSG00000119227), PTPRN2(ENSG00000155093), RSAD2(ENSG00000134321), SLC39A14(ENSG00000104635), SLC6A12(ENSG00000111181), SLC6A15(ENSG00000072041), RAB25(ENSG00000132698), SLC20A2(ENSG00000168575), SLC39A10(ENSG00000196950), TGFBR1(ENSG00000106799), STK16(ENSG00000115661), TRAF4(ENSG00000076604), SIGLEC14(ENSG00000254415), MTG2(ENSG00000101181), SPTLC3(ENSG00000172296), SEC11C(ENSG00000166562), MARK2(ENSG00000072518), SCN7A(ENSG00000136546), SLC15A3(ENSG00000110446), PNLDC1(ENSG00000146453), ARHGAP27(ENSG00000159314), RNF186(ENSG00000178828), TSPAN15(ENSG00000099282), TRAF3IP3(ENSG00000009790), VTCN1(ENSG00000134258), TAS2R38(ENSG00000257138), TCTN3(ENSG00000119977), UBL3(ENSG00000122042), TMEM39B(ENSG00000121775), SLC30A10(ENSG00000196660), CA2(ENSG00000104267), CDHR5(ENSG00000099834), CLDN4(ENSG00000189143), ARL4D(ENSG00000175906), ACVR1B(ENSG00000135503), GGT1(ENSG00000100031), DPEP1(ENSG00000015413), GSDMB(ENSG00000073605), GPBAR1(ENSG00000179921), GHR(ENSG00000112964), KCNJ8(ENSG00000121361), SYK(ENSG00000165025), FPR1(ENSG00000171051), ADCY10(ENSG00000143199), ACSL5(ENSG00000197142), CD177(ENSG00000204936), CLCA1(ENSG00000016490), GLP2R(ENSG00000065325), CADM4(ENSG00000105767), DSC2(ENSG00000134755), GNB5 (ENSG00000069966), ADGRF1(ENSG00000153292), RASGRP2(ENSG00000068831), CLDN5(ENSG00000184113), EPHA4(ENSG00000116106), HCAR3(ENSG00000255398), DSG2(ENSG00000046604), AQP8(ENSG00000103375), CNTFR(ENSG00000122756), CEACAMS(ENSG00000105388), CHST6(ENSG00000183196), CA4(ENSG00000167434), DEPDC5(ENSG00000100150), EPCAM(ENSG00000119888), GNG11(ENSG00000127920), LPAR6(ENSG00000139679), CTDNEP1(ENSG00000175826), CPTP(ENSG00000224051), HSD17B2(ENSG00000086696), MAGI1(ENSG00000151276), ENTPD8(ENSG00000188833), FPGS(ENSG00000136877), MAN1C1(ENSG00000117643), FADS3(ENSG00000221968), CXCRl(ENSG00000163464), FADS2(ENSG00000134824), FER1L6(ENSG00000214814), ANKRD13B(ENSG00000198720), BEST2(ENSG00000039987), BRI3(ENSG00000164713), CDC42EP5(ENSG00000167617), CLDN8(ENSG00000156284), GAL3ST2(ENSG00000154252), HTR4(ENSG00000164270), B3GNT8(ENSG00000177191),
ELOVL7(ENSG00000164181), B3GALT5(ENSG00000183778), CDHR2(ENSG00000074276), CLDN7(ENSG00000181885), CLN3(ENSG00000188603), CRB3(ENSG00000130545), ACE2(ENSG00000130234), SH3BP5(ENSG00000131370), ATP9A(ENSG00000054793), CNTNAP3(ENSG00000106714), CDH17(ENSG00000079112), C3AR1(ENSG00000171860), ATP11C(ENSG00000101974), KCNIP3(ENSG00000115041), ATP1A2(ENSG00000018625), CIB1(ENSG00000185043), ADGRG6(ENSG00000112414), ASIC5(ENSG00000256394), CEACAM6(ENSG00000086548), DCXR(ENSG00000169738), AKAP10(ENSG00000108599), ARL4C(ENSG00000188042), ANO1(ENSG00000131620), ANXA13(ENSG00000104537), CHST14(ENSG00000169105), ARHGEF4(ENSG00000136002), ABHD12(ENSG00000100997), C12orf66(ENSG00000174206), CNPPD1(ENSG0000011S649), BLNK(ENSG00000095585), ALG14(ENSG00000172339), CEACAM3(ENSG00000170956), HTR2B(ENSG00000135914), B3GAT1(ENSG00000109956), CASQ1(ENSG00000143318), HHLA2(ENSG00000114455), MAOA(ENSG00000189221), CYP2C9(ENSG00000138109), DIP2A(ENSG00000160305), FGFR4(ENSG00000160867), IBTK(ENSG00000005700), SLC9A3R2(ENSG00000065054), NCEH1(ENSG00000144959), LRFN5(ENSG00000165379), MYZAP(ENSG00000263155), LHFPL2(ENSG00000145685), KCNN3(ENSG00000143603), MS4A18(ENSG00000214782), MC4R(ENSG00000166603), MS4A12(ENSG00000071203), MCUB(ENSG00000005059), EPPKl(ENSG00000261150), MSTO1(ENSG00000125459), SLC2A6(ENSG00000160326), KIAA2013(ENSG00000116685), NIPAL1(ENSG00000163293), SLC16A10(ENSG00000112394), IL17RA(ENSG00000177663), ITGB4(ENSG00000132470), LY9(ENSG00000122224), LYN(ENSG00000254087), GPA33(ENSG00000143167), ALOX12(ENSG00000108839), MUC13(ENSG00000173702), OR10R2(ENSG00000198965), MRC1(ENSG00000260314), NECAP2(ENSG00000157191), KLRC3(ENSG00000205810), HCAR2(ENSG00000182782), PRKCA(ENSG00000154229), PSKH1(ENSG00000159792), NDST2(ENSG00000166507), TGM2(ENSG00000198959), VSIG4(ENSG00000155659), SLC30A4(ENSG00000104154), EFNB3(ENSG00000108947), CD300C(ENSG00000167850), SLC22A15(ENSG00000163393), ST6GAL1(ENSG00000073849), SCEL(ENSG00000136155), AGER(ENSG00000204305), PLLP(ENSG00000102934), SLC18A2(ENSG00000165646), REEP2(ENSG00000132563), MGAT4B(ENSG00000161013), SCTR(ENSG00000080293), EHD2(ENSG00000024422), PPL(ENSG00000118898), CD84(ENSG00000066294), TMEM80(ENSG00000177042), RHOG(ENSG00000177105), TM4SF1(ENSG00000169908), STEAP1(ENSG00000164647), EHD1(ENSG00000110047), GJB4(ENSG00000189433), CD247(ENSG00000198821), CCR7(ENSG00000126353), CYB5A(ENSG00000166347), HCLS1(ENSG00000180353), AIFM2(ENSG00000042286), AIGl(ENSG00000146416), APBB1IP(ENSG00000077420), AQP4(ENSG00000171885), CYB561D1(ENSG00000174151), CACNA2D2(ENSG00000007402), ACE(ENSG00000159640), AQP3(ENSG00000165272), CADM1(ENSG00000182985), CXCR3(ENSG00000186810), LAMP3(ENSG00000078081), EHD3(ENSG00000013016), NSMF(ENSG00000165802), SLC34A2(ENSG00000157765), LRRK2(ENSG00000188906), TTYH3(ENSG00000136295), SUN3(ENSG00000164744), VPS52(ENSG00000223501), CLEC10A(ENSG00000132514), CD300C(ENSG00000167850), CD47(ENSG00000196776), MGAT4C(ENSG00000182050), PERP(ENSG00000112378), SNTG2(ENSG00000172554), ALPL(ENSG00000162551), TMPRSS11D(ENSG00000153802), SLC6A1(ENSG00000157103), SCEL(ENSG00000136155), SMURF1(ENSG00000198742), SEC13(ENSG00000157020), SLC41A2(ENSG00000136052), TMEM97(ENSG00000109084), MPP7(ENSG00000150054), RAC1(ENSG00000136238), RNF145(ENSG00000145860), ARHGAP5(ENSG00000100852), NFAMl(ENSG00000235568), SLC26A9(ENSG00000174502), RAC3(ENSG00000169750), RAB27B(ENSG00000041353), MTMR2(ENSG00000087053), MUC1(ENSG00000185499), REEP2(ENSG00000132563), KCNH8(ENSG00000183960), GPIHBPl(ENSG00000277494), SDC1(ENSG00000115884), LTBR(ENSG00000111321), TMEM45A(ENSG00000181458), MGAT4B(ENSG00000161013), TYROBP(ENSG00000011600), STYK1(ENSG00000060140), TMEM134(ENSG00000172663), SLC10A6(ENSG00000145283), MAPKAP1(ENSG00000119487), IFI27(ENSG00000165949), SGMS1(ENSG00000198964), MUC15(ENSG00000169550), OLFM2(ENSG00000105088), PROM2(ENSG00000155066), PLPP7(ENSG00000160539), KRT1(ENSG00000167768), PLA2G4E(ENSG00000188089), LRRN2(ENSG00000170382), DMPK(ENSG00000104936), OBSCN(ENSG00000154358), PPL(ENSG00000118898), TMEM79(ENSG00000163472), SLC7A7(ENSG00000155465), YRDC(ENSG00000196449), ZP2(ENSG00000103310), SYTL3(ENSG00000164674), TACSTD2(ENSG00000184292), PRRG2(ENSG00000126460), MSLN(ENSG00000102854), OCLN(ENSG00000197822), SPHK1(ENSG00000176170), SLC28A3(ENSG00000197506), S1PR5(ENSG00000180739), PTAFR(ENSG00000169403), RAB25(ENSG00000132698), SLC20A2(ENSG00000168575), SLC39A10(ENSG00000196950), STK16(ENSG00000115661), SLC19A1(ENSG00000173638), MARK2(ENSG00000072518), PNLDC1(ENSG00000146453), TRAF3IP3(ENSG00000009790), TAS2R38(ENSG00000257138), TMEM39B(ENSG00000121775), CLDN4(ENSG00000189143), CYBA(ENSG00000051523), GJB4(ENSG00000189433), KCNJ8(ENSG00000121361), SYK(ENSG00000165025), FA2H(ENSG00000103089), FPR1(ENSG00000171051), DSP(ENSG00000096696), ADCY10(ENSG00000143199), CDH6(ENSG00000113361), CD177(ENSG00000204936), GLP2R(ENSG00000065325), ANO7(ENSG00000146205), FYN(ENSG00000010810), DSC2(ENSG00000134755), GPX8(ENSG00000164294), CLDNS(ENSG00000184113), EPHA4(ENSG00000116106), HCAR3(ENSG00000255398), DSG2(ENSG00000046604), GPR39(ENSG00000183840), CD248(ENSG00000174807), CAPNS2(ENSG00000256812), HCN1(ENSG00000164588), DSC3(ENSG00000134762), GSDMA(ENSG00000167914), FGFBP1(ENSG00000137440), CTDNEP1(ENSG00000175826), FADS3(ENSG00000221968), FADS2(ENSG00000134824), B3GNT4(ENSG00000176383), DSG1(ENSG00000134760), ADGRF4(ENSG00000153294), BRI3(ENSG00000164713), ELOVL7(ENSG00000164181), MS4A1(ENSG00000156738), ASPRV1(ENSG00000244617), CIB1(ENSG00000185043), ASIC5(ENSG00000256394), DSG3(ENSG00000134757), DCXR(ENSG00000169738), AQP3(ENSG00000165272), CLEC2A(ENSG00000188393), CNPPD1(ENSG00000115649), BLNK(ENSG00000095585), MAOA(ENSG00000189221), IBTK(ENSG00000005700), MYZAP(ENSG00000263155), MC4R(ENSG00000166603), MCUB(ENSG00000005059), EPPKl(ENSG00000261150), FGFR3(ENSG00000068078), MSTO1(ENSG00000125459), NIPAL1(ENSG00000163293), ITGB4(ENSG00000132470), ALOX12(ENSG00000108839), OR10R2(ENSG00000198965), HCAR2(ENSG00000182782), PSKH1(ENSG00000159792), SLC30A4(ENSG00000104154), MS4A8(ENSG00000166959), PI4KB(ENSG00000143393), ULBP3(ENSG00000131019), GPR65(ENSG00000140030), RHBG(ENSG00000132677), PLLP(ENSG00000102934), DLC1(ENSG00000164741), PLN(ENSG00000198523), OSBPL7(ENSG00000006025), CD46(ENSG00000117335), ICAM3(ENSG00000076662), SLC25A15(ENSG00000102743), TMEM80(ENSG00000177042), TMEM143(ENSG00000161558), UNC13B(ENSG00000198722), RHCG(ENSG00000140519), TPRA1(ENSG00000163870), SNTB1(ENSG00000172164), SH2D3C(ENSG00000095370), RNF170(ENSG00000120925), RHOG(ENSG00000177105), RSAD2(ENSG00000134321), S100A8(ENSG00000143546), RAB11FIP5(ENSG00000135631), TM4SF1(ENSG00000169908), TRAF4(ENSG00000076604), MTG2(ENSG00000101181), SEMA5B(ENSG00000082684), ARHGAP27(ENSG00000159314), TSPAN15(ENSG00000099282), VTCN1(ENSG00000134258), TCTN3(ENSG00000119977), EHD1(ENSG00000110047), GSDMC(ENSG00000147697), CYB5A(ENSG00000166347), EVI2A(ENSG00000126860), FCGR2B(ENSG00000072694), CNTFR(ENSG00000122756), CEACAM5(ENSG00000105388), GPAT2(ENSG00000186281), CHST6(ENSG00000183196), DNER(ENSG00000187957), FGD2(ENSG00000146192), CPTP(ENSG00000224051), CXCRl(ENSG00000163464), FNBP1(ENSG00000187239), B3GNT8(ENSG00000177191), BASPl(ENSG00000176788), ATP9A(ENSG00000054793), CYB561D1(ENSG00000174151), CDH13(ENSG00000140945), AKAP10(ENSG00000108599),
ANO1(ENSG00000131620), CHST14(ENSG00000169105), AP4E1(ENSG00000081014), ALG14(ENSG00000172339), HTR2B(ENSG00000135914), NCEH1(ENSG00000144959), LHFPL2(ENSG00000145685), MUC21(ENSG00000204544), EHD3(ENSG00000013016), IL17RA(ENSG00000177663), NECAP2(ENSG00000157191), (), UPK1B(ENSG00000114638), CLEC10A(ENSG00000132514), CREB3L3(ENSG00000060566), PI4KB(ENSG00000143393), SLC6A4(ENSG00000108576), SLC25A40(ENSG00000075303), SLCO1B3(ENSG00000111700), SLCO1B1(ENSG00000134538), SLC25A43(ENSG00000077713), SLC27A5(ENSG00000083807), MTMR2(ENSG00000087053), PTGDR2(ENSG00000183134), PDZK1(ENSG00000174827), RDH16(ENSG00000139547), LIN7A(ENSG00000111052), PRRT2(ENSG00000167371), OLFM2(ENSG00000105088), NAT8(ENSG00000144035), PNPLA3(ENSG00000100344), SLC25A15(ENSG00000102743), YIPFl(ENSG00000058799), TFR2(ENSG00000106327), UGT1A6(ENSG00000167165), TNS2(ENSG00000111077), ST7L(ENSG00000007341), SLCO2B1(ENSG00000137491), TEX261(ENSG00000144043), TSPAN2(ENSG00000134198), GLRB(ENSG00000109738), CYP2D6(ENSG00000100197), FYN(ENSG00000010810), GAS2(ENSG00000148935), SLC2A2(ENSG00000163581), CYP2A7(ENSG00000198077), FOLH1(ENSG00000086205), CYP1A2(ENSG00000140505), CYP3A4(ENSG00000160868), CYP4V2(ENSG00000145476), NINJ1(ENSG00000131669), CYP2C8(ENSG00000138115), DHCR24(ENSG00000116133), MAL2(ENSG00000147676), CYP2C19(ENSG00000165841), AQP9(ENSG00000103569), ASGR1(ENSG00000141505), ADRA2B(ENSG00000274286), ABCB11(ENSG00000073734), CMKLR1(ENSG00000174600), ACAD11(ENSG00000240303), BACE2(ENSG00000182240), CD320(ENSG00000167775), ASGR2(ENSG00000161944), PIK3AP1(ENSG00000155629), CYP3A43(ENSG00000021461), INTS2(ENSG00000108506), MOXD1(ENSG00000079931), MFAP3L(ENSG00000198948), KCND1(ENSG00000102057), GPR15(ENSG00000154165), SLC2A8(ENSG00000136856), ABCC2(ENSG00000023839), DPP4(ENSG00000197635), NPC1L1(ENSG00000015520), ZDHHC16(ENSG00000171307), CD47(ENSG00000196776), SLC45A1(ENSG00000162426), PODXL(ENSG00000128567), PECAM1(ENSG00000261371), SCUBE1(ENSG00000159307), PLA2R1(ENSG00000153246), MAP6(ENSG00000171533), MPP5(ENSG00000072415), NPHS2(ENSG00000116218), TSPAN7(ENSG00000156298), THSD7A(ENSG00000005108), PITPNM3(ENSG00000091622), PTPRO(ENSG00000151490), HYAL2(ENSG00000068001), CDH6(ENSG00000113361), CD93(ENSG00000125810), FAM107A(ENSG00000168309), CD34(ENSG00000174059), EVI2A(ENSG00000126860), ENG(ENSG00000106991), CD248(ENSG00000174807), DOCKS(ENSG00000147459), CR1(ENSG00000203710), FGD2(ENSG00000146192), FNBP1(ENSG00000187239), ACKR1(ENSG00000213088), CDH13(ENSG00000140945), SUN3(ENSG00000164744), TRPM3(ENSG00000083067), TBC1D3G(ENSG00000260287), TREH(ENSG00000118094), THY1(ENSG00000154096), SELPLG(ENSG00000110876), TMEM240(ENSG00000205090), SLC5A12(ENSG00000148942), SLC12A1(ENSG00000074803), SLC4A10(ENSG00000144290), SLC6A19(ENSG00000174358), SLC22A11(ENSG00000168065), S1PR2(ENSG00000267534), SLC28A2(ENSG00000137860), RDH11(ENSG00000072042), RHBG(ENSG00000132677), VHL(ENSG00000134086), PDE2A(ENSG00000186642), RALB(ENSG00000144118), LTBR(ENSG00000111321), TMEM132E(ENSG00000181291), NOS1(ENSG00000089250), NTRK2(ENSG00000148053), RFTN2(ENSG00000162944), SLC22A6(ENSG00000197901), UGT3A1(ENSG00000145626), ATP6V0A4(ENSG00000105929), TMEM252(ENSG00000181778), TMEM174(ENSG00000164325), RHCG(ENSG00000140519), FASLG(ENSG00000117560), SLC28A1(ENSG00000156222), SNTB1(ENSG00000172164), SIRT2(ENSG00000068903), SLC22A2(ENSG00000112499), SH2D3C(ENSG00000095370), SLC22A8(ENSG00000149452), RAB11FIP5(ENSG00000135631), SLC6A18(ENSG00000164363), SLC13A2(ENSG00000007216), TMEM213(ENSG00000214128), SLC5A1(ENSG00000100170), SYT7(ENSG00000011347), SLC4A4(ENSG00000080493), TMEM178B(ENSG00000261115), PKHD1(ENSG00000170927), SLC35G2(ENSG00000168917), TSPAN16(ENSG00000130167), ENPP6(ENSG00000164303), EMC4(ENSG00000128463), IYD(ENSG00000009765), ENPP3(ENSG00000154269), CUBN(ENSG00000107611), DLK1(ENSG00000185559), GSDMC(ENSG00000147697), KIRREL2(ENSG00000126259), CPNE6(ENSG00000100884), JAKMIP1(ENSG00000152969), CLTRN(ENSG00000147003), CPT1C(ENSG00000169169), CLCNKB(ENSG00000184908), ADGRG5(ENSG00000159618), CALCR(ENSG00000004948), CLCNKA(ENSG00000186510), FAM169A(ENSG00000198780), GPR39(ENSG00000183840), CASR(ENSG00000036828), GLIPR1L2(ENSG00000180481), LPXN(ENSG00000110031), KIAA0319(ENSG00000137261), LRFN4(ENSG00000173621), AQP6(ENSG00000086159), DSG1(ENSG00000134760), NPR3(ENSG00000113389), CDC42SE1(ENSG00000197622), ADAM28(ENSG00000042980), SLC7A9(ENSG00000021488), CLDN10(ENSG00000134873), DUSP15(ENSG00000149599), FXYD2(ENSG00000137731), CLDN2(ENSG00000165376), ANO5(ENSG00000171714), AP4E1(ENSG00000081014), CDH16(ENSG00000166589), LRFN2(ENSG00000156564), KCNJ10(ENSG00000177807), KCNJ12(ENSG00000184185), MS4A4A(ENSG00000110079), FMO1(ENSG00000010932), LRP2(ENSG00000081479), IL23R(ENSG00000162594), GRIN2D(ENSG00000105464), MDGA2(ENSG00000139915), KCTD8(ENSG00000183783), SLC27A6(ENSG00000113396), SPAG4(ENSG00000061656), UCP3(ENSG00000175564), RASL10B(ENSG00000270885), POPDC2(ENSG00000121577), PLN(ENSG00000198523), GPIHBP1(ENSG00000277494), INPP5D(ENSG00000168918), SGCG(ENSG00000102683), SRL(ENSG00000185739), ART3(ENSG00000156219), KLHL41(ENSG00000239474), DMPK(ENSG00000104936), YRDC(ENSG00000196449), TREML1(ENSG00000161911), RAB11FIP4(ENSG00000131242), RYR1(ENSG00000196218), SLAMF8(ENSG00000158714), SEMA5B(ENSG00000082684), MMP27(ENSG00000137675), DSP(ENSG00000096696), CDH15(ENSG00000129910), ADRA1A(ENSG00000120907), GPX8(ENSG00000164294), CAVIN4(ENSG00000170681), FCGR2B(ENSG00000072694), CHRNA1(ENSG00000138435), GPAT2(ENSG00000186281), LEPROTL1(ENSG00000104660), B3GNT4(ENSG00000176383), CSF3R(ENSG00000119535), ADRB1(ENSG00000043591), JPH1(ENSG00000104369), GPR182(ENSG00000166856), MCEMP1(ENSG00000183019), DES(ENSG00000175084), TMEM100(ENSG00000166292), SLITRK4(ENSG00000179542), SLC5A5(ENSG00000105641), KCNQ5(ENSG00000185760), TPO(ENSG00000115705), PSD3(ENSG00000156011), PORCN(ENSG00000102312), C19orfl8(ENSG00000177025), DUOX1(ENSG00000137857), EPHX4(ENSG00000172031), FCRL1(ENSG00000163534), HCN1(ENSG00000164588), IPCEF1(ENSG00000074706), ADD2(ENSG00000075340), BASP1(ENSG00000176788), AAK1(ENSG00000115977), CRHR1(ENSG00000120088), CDH10(ENSG00000040731), LRP1B(ENSG00000168702), RAB3A(ENSG00000105649), RTN1(ENSG00000139970), SCG3(ENSG00000104112), POMGNT2(ENSG00000144647), PTPRN(ENSG00000054356), NECAB2(ENSG00000103154), SLC17A6(ENSG00000091664), SV2A(ENSG00000159164), SLC30A8(ENSG00000164756), MAPK10(ENSG00000109339), DGCR2(ENSG00000070413), GNAO1(ENSG00000087258), CLU(ENSG00000120885), AMPH(ENSG00000078053), GAD2(ENSG00000136750), ALOX5(ENSG00000012779), TBC1D3D(ENSG00000274419), TMEM97(ENSG00000109084), SEC16B(ENSG00000120341), UNC13D(ENSG00000092929), TRAT1(ENSG00000163519), TBC1D3E(ENSG00000278599), TACSTD2(ENSG00000184292), SYCN(ENSG00000179751), SLC19A1(ENSG00000173638), CUZD1(ENSG00000138161), GP2(ENSG00000169347), FA2H(ENSG00000103089), CYSTM1(ENSG00000120306), CDH9(ENSG00000113100), CFTR(ENSG00000001626), CHST4(ENSG00000140835), AQP12A(ENSG00000184945), AQP12B(ENSG00000185176), TMEM158(ENSG00000249992), UGT2A3(ENSG00000135220), CACNB2(ENSG00000165995), CORO7(ENSG00000262246), ABCD4(ENSG00000119688), GABARAPL2(ENSG00000034713), UBE2J1(ENSG00000198833), SYT13(ENSG00000019505), PLSCR4(ENSG00000114698), SPCS1(ENSG00000114902), TNFSF12(ENSG00000239697), BVES(ENSG00000112276), P2RX6(ENSG00000099957), PLEKHA3(ENSG00000116095), TVP23B(ENSG00000171928),
WHAMM(ENSG00000156232), PTPRD(ENSG00000153707), MED28(ENSG00000118579), PTPRT(ENSG00000196090), MRGPRF(ENSG00000172935), SPATA9(ENSG00000145757), SLCO4A1(ENSG00000101187), SLC9A3(ENSG00000066230), ADCYAP1R1(ENSG00000078549), MRGPRX2(ENSG00000183695), PCDH11X(ENSG00000102290), PALM(ENSG00000099864), VEPH1(ENSG00000197415), PRKAR2B(ENSG00000005249), CNEP1R1(ENSG00000205423), SORD(ENSG00000140263), GOLGA2(ENSG00000167110), AATK(ENSG00000181409), SPRED1(ENSG00000166068), SLC26A3(ENSG00000091138), UGT2B17(ENSG00000197888), SERAC1(ENSG00000122335), TVP23C(ENSG00000175106), UGT2B28(ENSG00000135226), VAC14(ENSG00000103043), QTRT1(ENSG00000213339), TSPAN13(ENSG00000106537), TMEM253(ENSG00000232070), TBCD(ENSG00000141556), UGT2B15(ENSG00000196620), ARHGAP17(ENSG00000140750), SEZ6(ENSG00000063015), C2orf88(ENSG00000187699), GOPC(ENSG00000047932), SNX6(ENSG00000129515), PHACTR2(ENSG00000112419), RAB29(ENSG00000117280), NAPG(ENSG00000134265), PHLPP2(ENSG00000040199), PRRT3(ENSG00000163704), SLC35B1(ENSG00000121073), NAPB(ENSG00000125814), TMEM154(ENSG00000170006), TMEM171(ENSG00000157111), TMEM30A(ENSG00000112697), CLSTN1(ENSG00000171603), FCER1A(ENSG00000179639), IL9R(ENSG00000124334), SLC2A12(ENSG00000146411), CEACAM1(ENSG00000079385), FPR2(ENSG00000171049), FKBP2(ENSG00000173486), GRK5(ENSG00000198873), CHDH(ENSG00000016391), FAM126B(ENSG00000155744), HTR1A(ENSG00000178394), GLT8D2(ENSG00000120820), ENOX2(ENSG00000165675), GATM(ENSG00000171766), FLNC(ENSG00000128591), GCHFR(ENSG00000137880), AADAC(ENSG00000114771), DIAPH1(ENSG00000131504), ADGRE1(ENSG00000174837), CHODL(ENSG00000154645), CA12(ENSG00000074410), DPP10(ENSG00000175497), DGKQ(ENSG00000145214), GBP2(ENSG00000162645), LRRC37B(ENSG00000185158), AP3M1(ENSG00000185009), ABHD16A(ENSG00000204427), ANKRD27(ENSG00000105186), ANO8(ENSG00000074855), AQP1(ENSG00000240583), MPP1(ENSG00000130830), CDC42EP1(ENSG00000128283), UGCG(ENSG00000148154), ENPEP(ENSG00000138792), CLDN11(ENSG00000013297), ARRB1(ENSG00000137486), CADPS(ENSG00000163618), C5AR2(ENSG00000134830), ARRDC1(ENSG00000197070), AP1S3(ENSG00000152056), EPB41L3(ENSG00000082397), ADGRV1(ENSG00000164199), EBP(ENSG00000147155), HSD3B7(ENSG00000099377), ABCC5(ENSG00000114770), SMPD4(ENSG00000136699), HMOX1(ENSG00000100292), SLC16A6(ENSG00000108932), NDUFS1(ENSG00000023228), GFRA2(ENSG00000168546), GNG12(ENSG00000172380), GLRA2(ENSG00000101958), MAP2K1(ENSG00000169032), NIPA2(ENSG00000140157), IGFLR1(ENSG00000126246), GAL3ST1(ENSG00000128242), DPY19L1(ENSG00000173852), RPS6KC1(ENSG00000136643), TRIM13(ENSG00000204977), SLC25A17(ENSG00000100372), TGFBR3(ENSG00000069702), SEMA4F(ENSG00000135622), NUS1(ENSG00000153989), PTPRG(ENSG00000144724), SGIP1(ENSG00000118473), KIRREL3(ENSG00000149571), TBC1D3(ENSG00000274611), P2RY2(ENSG00000175591), PSD4(ENSG00000125637), RYK(ENSG00000163785), SIGLEC9(ENSG00000129450), EHD4(ENSG00000103966), SLC44A2(ENSG00000129353), AOC3(ENSG00000131471), FES(ENSG00000182511), L1CAM(ENSG00000198910), ELMO2(ENSG00000062598), BSN(ENSG00000164061), BDKRB2(ENSG00000168398), LDLRAD2(ENSG00000187942), PI4KA(ENSG00000241973), CACNB2(ENSG00000165995), ABCD4(ENSG00000119688), GABARAPL2(ENSG00000034713), SOX10(ENSG00000100146), SLC25A17(ENSG00000100372), TGFBR3(ENSG00000069702), SPCS1(ENSG00000114902), TNFSF12(ENSG00000239697), NEGR1(ENSG00000172260), NUS1(ENSG00000153989), SLC9A3(ENSG00000066230), LANCL2(ENSG00000132434), PALM(ENSG00000099864), LAX1(ENSG00000122188), VEPH1(ENSG00000197415), SORD(ENSG00000140263), GOLGA2(ENSG00000167110), AATK(ENSG00000181409), TRPM2(ENSG00000142185), RMC1(ENSG00000141452), SERAC1(ENSG00000122335), VAC14(ENSG00000103043), TBCD(ENSG00000141556), UGT2B15(ENSG00000196620), P2RY2(ENSG00000175591), ARHGAP17(ENSG00000140750), C2orf88(ENSG00000187699), GOPC(ENSG00000047932), PHACTR2(ENSG00000112419), RAB29(ENSG00000117280), RYK(ENSG00000163785), PRRT3(ENSG00000163704), NAPB(ENSG00000125814), SIGLEC9(ENSG00000129450), TMEM171(ENSG00000157111), TMEM30A(ENSG00000112697), CACNA2D1(ENSG00000153956), FPR2(ENSG00000171049), FKBP2(ENSG00000173486), GRK5(ENSG00000198873), FAM126B(ENSG00000155744), GLT8D2(ENSG00000120820), ENOX2(ENSG00000165675), GATM(ENSG00000171766), AADAC(ENSG00000114771), CD99(ENSG00000002586), CA12(ENSG00000074410), MALL(ENSG00000144063), DGKQ(ENSG00000145214), GBP2(ENSG00000162645), LRRC37B(ENSG00000185158), L1CAM(ENSG00000198910), AP3M1(ENSG00000185009), ANKRD27(ENSG00000105186), CDC42EP1(ENSG00000128283), CD81(ENSG00000110651), BST1(ENSG00000109743), ARRB1(ENSG00000137486), CD1D(ENSG00000158473), ADGRV1(ENSG00000164199), HSD3B7(ENSG00000099377), ABCCS(ENSG00000114770), GNG12(ENSG00000172380), MAP2K1(ENSG00000169032), NIPA2(ENSG00000140157), LDLRAD2(ENSG00000187942), RPS6KC1(ENSG00000136643), CHL1(ENSG00000134121), PI4KA(ENSG00000241973), NCS1(ENSG00000107130), TCTN2(ENSG00000168778), CORO7(ENSG00000262246), TRIM13(ENSG00000204977), UBE2J1(ENSG00000198833), SYT13(ENSG00000019505), TYR(ENSG00000077498), WIPI2(ENSG00000157954), MPP6(ENSG00000105926), PTPRT(ENSG00000196090), SLCO4A1(ENSG00000101187), KCNQ4(ENSG00000117013), SGIP1(ENSG00000118473), PRKAR2B(ENSG00000005249), SPRED1(ENSG00000166068), QTRT1(ENSG00000213339), TSPAN13(ENSG00000106537), TBC1D3(ENSG00000274611), DCT(ENSG00000080166), ZC4H2(ENSG00000126970), NAPG(ENSG00000134265), PMEL(ENSG00000185664), PHLPP2(ENSG00000040199), DTL(ENSG00000143476), CNTN3(ENSG00000113805), CLSTN1(ENSG00000171603), FCER1A(ENSG00000179639), EHD4(ENSG00000103966), ADCY7(ENSG00000121281), TM7SF2(ENSG00000149809), CYB561A3(ENSG00000162144), DIAPH1(ENSG00000131504), FAM210A(ENSG00000177150), ADGRE1(ENSG00000174837), CD82(ENSG00000085117), PROCR(ENSG00000101000), UGCG(ENSG00000148154), ELMO2(ENSG00000062598), CPNE2(ENSG00000140848), BDKRB2(ENSG00000168398), ADGRA1(ENSG00000197177), NDUFS1(ENSG00000023228), GFRA2(ENSG00000168546), VNN1(ENSG00000112299), SVEP1(ENSG00000165124), NEGR1(ENSG00000172260), KSR2(ENSG00000171435), KCNQ4(ENSG00000117013), MME(ENSG00000196549), ZDHHC13(ENSG00000177054), SLC25A25(ENSG00000148339), CACNA2D1(ENSG00000153956), CREB3L2(ENSG00000182158), HEPACAM(ENSG00000165478), FAM210A(ENSG00000177150), FMO3(ENSG00000007933), MALL(ENSG00000144063), ABCB4(ENSG00000005471), KMO(ENSG00000117009), BCL2L10(ENSG00000137875), CTSL(ENSG00000135047), HSD11B1(ENSG00000117594), LIMS2(ENSG00000072163), SLC10A1(ENSG00000100652), ZAP70(ENSG00000115085), KDR(ENSG00000128052), MAPT(ENSG00000186868), KIRREL1(ENSG00000183853), SLC34A1(ENSG00000131183), PTH2R(ENSG00000144407), NKD1(ENSG00000140807), RMC1(ENSG00000141452), TTC7B(ENSG00000165914), STK10(ENSG00000072786), SNCA(ENSG00000145335), PEAR1(ENSG00000187800), RTL1(ENSG00000254656), CD99(ENSG00000002586), MAS1(ENSG00000130368), ADGRL4(ENSG00000162618), CHRM1(ENSG00000168539), SLC43A2(ENSG00000167703), ITGA8(ENSG00000077943), MLIP(ENSG00000146147), PDGFRA(ENSG00000134853), SLC5A2(ENSG00000140675), STIMATE(ENSG00000213533), TLN2(ENSG00000171914), PCDHB2(ENSG00000112852), SCNN1D(ENSG00000162572), SLC34A3(ENSG00000198569), PTH1R(ENSG00000160801), WIPI2(ENSG00000157954), MPP6(ENSG00000105926), PRICKLE1(ENSG00000139174), TRPM2(ENSG00000142185), XPNPEP2(ENSG00000122121), SLC13A3(ENSG00000158296), OPRD1(ENSG00000116329), CLSTN2(ENSG00000158258), CKMT2(ENSG00000131730), ADCY7(ENSG00000121281), FAM151A(ENSG00000162391), FNDC5(ENSG00000160097), DOCK2(ENSG00000134516),
TM7SF2(ENSG00000149809), FRRS1L(ENSG00000260230), EPHB1(ENSG00000154928), BSND(ENSG00000162399), BST1(ENSG00000109743), CATIP(ENSG00000158428), CPT1B(ENSG00000205560), COX7B(ENSG00000131174), CPNE2(ENSG00000140848), C5AR1(ENSG00000197405), FAM234B(ENSG00000084444), MFSD1(ENSG00000118855), CHL1(ENSG00000134121), TCTN2(ENSG00000168778), CAP2(ENSG00000112186), SGCB(ENSG00000163069), P2RY6(ENSG00000171631), SHISA4(ENSG00000198892), FLT1(ENSG00000102755), LANCL2(ENSG00000132434), SIPA1(ENSG00000213445), ZC4H2(ENSG00000126970), CNTN3(ENSG00000113805), SLC2A4(ENSG00000181856), HHATL(ENSG00000010282), CYB561A3(ENSG00000162144), CAMK2B(ENSG00000058404), MBOAT7(ENSG00000125505), FCGRT(ENSG00000104870), DMD(ENSG00000198947), CD81(ENSG00000110651), CD1D(ENSG00000158473), AKAP6(ENSG00000151320), NCAM1(ENSG00000149294), IL18RAP(ENSG00000115607), VPS33B(ENSG00000184056), RTP5(ENSG00000188011), RNFT2(ENSG00000135119), PROCR(ENSG00000101000), AP3B2(ENSG00000103723), MTNR1B(ENSG00000134640), NCS1(ENSG00000107130), SHISAL2B(ENSG00000145642), FBXO2(ENSG00000116661), CD82(ENSG00000085117), NEURL1(ENSG00000107954), DTL(ENSG00000143476), TRDN(ENSG00000186439), TJAP1(ENSG00000137221), TMEM138(ENSG00000149483), TECR(ENSG00000099797), TMEM237(ENSG00000155755), TMCC1(ENSG00000172765), SLC7A6(ENSG00000103064), ATP8B2(ENSG00000143515), SLC46A1(ENSG00000076351), ST3GAL6(ENSG00000064225), SLC14A1(ENSG00000141469), TMEM106B(ENSG00000106460), RHOV(ENSG00000104140), LPCAT2(ENSG00000087253), SLC5A6(ENSG00000138074), SUSD3(ENSG00000157303), SLC6A6(ENSG00000131389), PITPNM1(ENSG00000110697), PEX1(ENSG00000127980), PEMT(ENSG00000133027), NPDC1(ENSG00000107281), NTRK1(ENSG00000198400), RALGPS2(ENSG00000116191), GOLGA8B(ENSG00000215252), GRIK2(ENSG00000164418), PCDHGA10(ENSG00000253846), RHOF(ENSG00000139725), HRAS(ENSG00000174775), PLA2G4F(ENSG00000168907), PILRA(ENSG00000085514), PKHD1L1(ENSG00000205038), RGS16(ENSG00000143333), USP8(ENSG00000138592), VPS26B(ENSG00000151502), YIF1B(ENSG00000167645), RHBDL2(ENSG00000158315), SLC25A20(ENSG00000178537), PLA2G2A(ENSG00000188257), KRAS(ENSG00000133703), STXBP6(ENSG00000168952), NRAS(ENSG00000213281), TENM3(ENSG00000218336), STXBP3(ENSG00000116266), TAOK2(ENSG00000149930), MITD1(ENSG00000158411), PRSS8(ENSG00000052344), GNPNAT1(ENSG00000100522), LRRC55(ENSG00000183908), NUTF2(ENSG00000102898), KCNQ1(ENSG00000053918), SLC8A1(ENSG00000183023), NAALADL2(ENSG00000177694), NECTIN1(ENSG00000110400), LDLRAD3(ENSG00000179241), NALCN(ENSG00000102452), SLC51B(ENSG00000186198), SLC43A3(ENSG00000134802), SELENOT(ENSG00000198843), SLC9A1(ENSG00000090020), ST7(ENSG00000004866), SNTA1(ENSG00000101400), SCARF1(ENSG00000074660), TUB(ENSG00000166402), TEX264(ENSG00000164081), TM4SF20(ENSG00000168955), TMEM119(ENSG00000183160), ZNRF1(ENSG00000186187), TMEM69(ENSG00000159596), ZFYVE9(ENSG00000157077), SLC35A3(ENSG00000117620), PCDH1(ENSG00000156453), RNF125(ENSG00000101695), TMTC4(ENSG00000125247), QPCTL(ENSG00000011478), SLC45A4(ENSG00000022567), RAB27A(ENSG00000069974), SLC12A6(ENSG00000140199), SORT1(ENSG00000134243), PARD3B(ENSG00000116117), SMIM6(ENSG00000259120), MIA3(ENSG00000154305), STK17B(ENSG00000081320), SSTR1(ENSG00000139874), TMEM204(ENSG00000131634), PPM1L(ENSG00000163590), SNX18(ENSG00000178996), SLC23A1(ENSG00000170482), SH3KBP1(ENSG00000147010), PIK3R5(ENSG00000141506), RHOU(ENSG00000116574), SLC4A5(ENSG00000188687), MSMO1(ENSG00000052802), TMIGD2(ENSG00000167664), TYRO3(ENSG00000092445), TRPC6(ENSG00000137672), ZBED3(ENSG00000132846), PRKCZ(ENSG00000067606), EMC9(ENSG00000100908), FAM83B(ENSG00000168143), FKRP(ENSG00000181027), FMOS(ENSG00000131781), EMC6(ENSG00000127774), KCTD3(ENSG00000136636), ARHGEF1(ENSG00000076928), GOLPH3(ENSG00000113384), ATP2C2(ENSG00000064270), ABCG2(ENSG00000118777), GPC5(ENSG00000179399), DCBLD1(ENSG00000164465), EPHB3(ENSG00000182580), CYP4F12(ENSG00000186204), CYP4F3(ENSG00000186529), CAPRIN2(ENSG00000110888), DGKE(ENSG00000153933), GCSAM(ENSG00000174500), CALN1(ENSG00000183166), CYP4F11(ENSG00000171903), GBF1(ENSG00000107862), DMTN(ENSG00000158856), GOLGA8A(ENSG00000175265), ATP11B(ENSG00000058063), WDPCP(ENSG00000143951), CNR1(ENSG00000118432), ARFGEF3(ENSG00000112379), CARMIL2(ENSG00000159753), CHMP5(ENSG00000086065), KPNA2(ENSG00000182481), FAM241B(ENSG00000171224), GDAP1(ENSG00000104381), GPR82(ENSG00000171657), IGSF11(ENSG00000144847), IL17RC(ENSG00000163702), EVA1B(ENSG00000142694), SLC7A8(ENSG00000092068), LPP(ENSG00000145012), EPHA10(ENSG00000183317), ATP5F1C(ENSG00000165629), CYP4F2(ENSG00000186115), KDELR3(ENSG00000100196), FERMT1(ENSG00000101311), ABCA10(ENSG00000154263), CD300LF(ENSG00000186074), CDH5(ENSG00000179776), DTX3L(ENSG00000163840), CYB561(ENSG00000008283), SLC25A31(ENSG00000151475), ATP2A2(ENSG00000174437), AVEN(ENSG00000169857), CERS6(ENSG00000172292), CD300A(ENSG00000167851), CXADR(ENSG00000154639), GRAMD1A(ENSG00000089351), ORAI1(ENSG00000276045), ACKR2(ENSG00000144648), CHST5(ENSG00000135702), B3GNT7(ENSG00000156966), CHMP2B(ENSG00000083937), ALDH3A2(ENSG00000072210), NISCH(ENSG00000010322), GIPC1(ENSG00000123159), GPR153(ENSG00000158292), FUT3(ENSG00000171124), GLUL(ENSG00000135821), HSP90AA1(ENSG00000080824), IL17RE(ENSG00000163701), KIAA1109(ENSG00000138688), MARCKSL1(ENSG00000175130), MTCH2(ENSG00000109919), EFHD2(ENSG00000142634), FUT6(ENSG00000156413), GPER1(ENSG00000164850), SLC2A3(ENSG00000059804), HCN3(ENSG00000143630), MBTPS1(ENSG00000140943), JAML(ENSG00000160593), GPC1(ENSG00000063660), LZTR1(ENSG00000099949), NFXL1(ENSG00000170448), MYOC(ENSG00000034971), NEU4(ENSG00000204099), MUC4(ENSG00000145113), NAPEPLD(ENSG00000161048), IL4R(ENSG00000077238), TRPV6(ENSG00000165125), STX5(ENSG00000162236), S100A9(ENSG00000163220), ECEL1(ENSG00000171551), CLDN18(ENSG00000066405), EPHX1(ENSG00000143819), CCDC88A(ENSG00000115355), CLIC2(ENSG00000155962), DGKZ(ENSG00000149091), GALNT13(ENSG00000144278), TMEM243(ENSG00000135185), TRDN(ENSG00000186439), TJAP1(ENSG00000137221), TMEM138(ENSG00000149483), TMEM237(ENSG00000155755), TMCC1(ENSG00000172765), ST3GAL6(ENSG00000064225), STRA6(ENSG00000137868), RXFP1(ENSG00000171509), RHOV(ENSG00000104140), SLC5A6(ENSG00000138074), SLC6A6(ENSG00000131389), PITPNM1(ENSG00000110697), NPDC1(ENSG00000107281), RALGPS2(ENSG00000116191), GRIK2(ENSG00000164418), NTM(ENSG00000182667), PTGS2(ENSG00000073756), PKHD1L1(ENSG00000205038), RGS16(ENSG00000143333), TMEM40(ENSG00000088726), VPS26B(ENSG00000151502), RHBDL2(ENSG00000158315), STX5(ENSG00000162236), PCDH19(ENSG00000165194), TENM3(ENSG00000218336), STXBP3(ENSG00000116266), TAOK2(ENSG00000149930), MITD1(ENSG00000158411), NEU3(ENSG00000162139), KCNAB1(ENSG00000169282), LRRC55(ENSG00000183908), NUTF2(ENSG00000102898), LYPD3(ENSG00000124466), SLC8A1(ENSG00000183023), NAALADL2(ENSG00000177694), NECTIN1(ENSG00000110400), SLC43A3(ENSG00000134802), SELENOT(ENSG00000198843), SLC9A1(ENSG00000090020), SNTA1(ENSG00000101400), SRC(ENSG00000197122), TUB(ENSG00000166402), ZNRF1(ENSG00000186187), TMEM69(ENSG00000159596), THBD(ENSG00000178726), SLC35A3(ENSG00000117620), RNF125(ENSG00000101695), TMTC4(ENSG00000125247), QPCTL(ENSG00000011478), SLC45A4(ENSG00000022567), PARD3B(ENSG00000116117), SMIM6(ENSG00000259120), MIA3(ENSG00000154305), NGFR(ENSG00000064300), SSTR1(ENSG00000139874), TMEM204(ENSG00000131634), SNX18(ENSG00000178996), SNORC(ENSG00000182600), SH3KBP1(ENSG00000147010),
LY6D(ENSG00000167656), S100A9(ENSG00000163220), RHOU(ENSG00000116574), SELENOK(ENSG00000113811), MSMO1(ENSG00000052802), RIPK4(ENSG00000183421), TYRO3(ENSG00000092445), TRPC6(ENSG00000137672), COL17A1(ENSG00000065618), EMC9(ENSG00000100908), FAM83B(ENSG00000168143), ARHGEF1(ENSG00000076928), CYP2W1(ENSG00000073067), ATP2C2(ENSG00000064270), GPC5(ENSG00000179399), DCBLD1(ENSG00000164465), FRS3(ENSG00000137218), EPHB3(ENSG00000182580), CYP4F12(ENSG00000186204), CYP4F3(ENSG00000186529), GPR84(ENSG00000139572), CALN1(ENSG00000183166), DMTN(ENSG00000158856), FAT2(ENSG00000086570), IL1RAP(ENSG00000196083), WDPCP(ENSG00000143951), ARFGEF3(ENSG00000112379), CARMIL2(ENSG00000159753), KPNA2(ENSG00000182481), GDAP1(ENSG00000104381), DEF6(ENSG00000023892), EVA1B(ENSG00000142694), SLC7A8(ENSG00000092068), LPP(ENSG00000145012), EPHA10(ENSG00000183317), KDELR3(ENSG00000100196), FERMT1(ENSG00000101311), ABCA10(ENSG00000154263), CCR2(ENSG00000121807), CDH5(ENSG00000179776), DTX3L(ENSG00000163840), CYB561(ENSG00000008283), SLC25A31(ENSG00000151475), AVEN(ENSG00000169857), CAV3(ENSG00000182533), CXADR(ENSG00000154639), ORAI1(ENSG00000276045), ACSBG1(ENSG00000103740), ACKR2(ENSG00000144648), ATP12A(ENSG00000075673), ALDH3A2(ENSG00000072210), KCNJ11(ENSG00000187486), NISCH(ENSG00000010322), GIPC1(ENSG00000123159), GPR153(ENSG00000158292), IL17RE(ENSG00000163701), KIAA1109(ENSG00000138688), MARCKSL1(ENSG00000175130), GPER1(ENSG00000164850), TACR1(ENSG00000115353), GPC1(ENSG00000063660), IL4R(ENSG00000077238), TMEM243(ENSG00000135185), TECR(ENSG00000099797), LPCAT2(ENSG00000087253), SUSD3(ENSG00000157303), PEMT(ENSG00000133027), NTRK1(ENSG00000198400), GOLGA8B(ENSG00000215252), HRAS(ENSG00000174775), PLA2G4F(ENSG00000168907), USP8(ENSG00000138592), YIF1B(ENSG00000167645), KRAS(ENSG00000133703), RASA4(ENSG00000105808), NRAS(ENSG00000213281), TECTA(ENSG00000109927), GNPNAT1(ENSG00000100522), KCNQ1(ENSG00000053918), ST7(ENSG00000004866), SMIM18(ENSG00000253457), TMEM119(ENSG00000183160), PRNP(ENSG00000171867), PCDH1(ENSG00000156453), STK17B(ENSG00000081320), TMIGD2(ENSG00000167664), UPK3A(ENSG00000100373), KCTD3(ENSG00000136636), IGSF3(ENSG00000143061), GBF1(ENSG00000107862), GOLGA8A(ENSG00000175265), CNR1(ENSG00000118432), FAM241B(ENSG00000171224), IGSF11(ENSG00000144847), IL17RC(ENSG00000163702), ATP5F1C(ENSG00000165629), DGKZ(ENSG00000149091), ATP2A2(ENSG00000174437), CERS6(ENSG00000172292), ATG2A(ENSG00000110046), B3GNT7(ENSG00000156966), CHMP2B(ENSG00000083937), LHFPL6(ENSG00000183722), EFHD2(ENSG00000142634), JAML(ENSG00000160593), KIT(ENSG00000157404), LZTR1(ENSG00000099949), KIAA0040(ENSG00000235750), STRA6(ENSG00000137868), SYNE3(ENSG00000176438), RASA4(ENSG00000105808), PCDH19(ENSG00000165194), RFTN1(ENSG00000131378), TM4SF4(ENSG00000169903), CYP2W1(ENSG00000073067), GJA5(ENSG00000265107), CACNA1A(ENSG00000141837), CYP2A6(ENSG00000255974), GPRC5B(ENSG00000167191), GPAM(ENSG00000119927), CYP2A13(ENSG00000197838), CYP2E1(ENSG00000130649), EPB41L5(ENSG00000115109), ATG2A(ENSG00000110046), KCNJ11(ENSG00000187486), PDGFRB(ENSG00000113721), TECTA(ENSG00000109927), MCAM(ENSG00000076706), NEU3(ENSG00000162139), KCNAB1(ENSG00000169282), MAST1(ENSG00000105613), NOS3(ENSG00000164867), SMIM18(ENSG00000253457), CBL(ENSG00000110395), BBS7(ENSG00000138686), KIAA0040(ENSG00000235750), TMEM231(ENSG00000205084), PTGER3(ENSG00000050628), RHBDD2(ENSG00000005486), PANX2(ENSG00000073150), ITPR1(ENSG00000150995), NKAIN4(ENSG00000101198), SRC(ENSG00000197122), SEMA6B(ENSG00000167680), SLC52A3(ENSG00000101276), TMEM184A(ENSG00000164855), SNORC(ENSG00000182600), RIPK4(ENSG00000183421), TMEM242(ENSG00000215712), UPK3A(ENSG00000100373), ERMP1(ENSG00000099219), IL1RAP(ENSG00000196083), CCR2(ENSG00000121807), CAV3(ENSG00000182533), ATP12A(ENSG00000075673), NXPE2(ENSG00000204361), PLCH2(ENSG00000149527), RYR2(ENSG00000198626), FRS3(ENSG00000137218), GPR84(ENSG00000139572), FAM168B(ENSG00000152102), ATP1A3(ENSG00000105409), TMEM251(ENSG00000153485), TMEM40(ENSG00000088726), NMUR2(ENSG00000132911), SYN3(ENSG00000185666), FZD5(ENSG00000163251), STXBP1(ENSG00000136854), PRNP(ENSG00000171867), SELENOK(ENSG00000113811), AQP5(ENSG00000161798), TMEM60(ENSG00000135211), ILK(ENSG00000166333), SLC47A1(ENSG00000142494), RAB17(ENSG00000124839), ENPP1(ENSG00000197594), SH3BP4(ENSG00000130147), SLC19A3(ENSG00000135917), MGAM(ENSG00000257335), PEX11G(ENSG00000104883), RNF144B(ENSG00000137393), SYBU(ENSG00000147642), MYORG(ENSG00000164976), MCOLN1(ENSG00000090674), RNF112(ENSG00000128482), UST(ENSG00000111962), SLC16A14(ENSG00000163053), RAB43(ENSG00000172780), RUFY3(ENSG00000018189), ARHGAP21(ENSG00000107863), TMEM219(ENSG00000149932), VASN(ENSG00000168140), CRK(ENSG00000167193), CSK(ENSG00000103653), AIF1L(ENSG00000126878), BMPR1A(ENSG00000107779), AP4M1(ENSG00000221838), AKR1A1(ENSG00000117448), CYP4X1(ENSG00000186377), KCNMA1(ENSG00000156113), ABCG8(ENSG00000143921), BCL2(ENSG00000171791), AP1S2(ENSG00000182287), ANPEP(ENSG00000166825), BBS1(ENSG00000174483), ASPHD1(ENSG00000174939), PAM(ENSG00000145730), ALOX15B(ENSG00000179593), NIPAL4(ENSG00000172548), HPS6(ENSG00000166189), IL10RB(ENSG00000243646), MXRA7(ENSG00000182534), ANK2(ENSG00000145362), SLC47A1(ENSG00000142494), RAB17(ENSG00000124839), ENPP1(ENSG00000197594), SH3BP4(ENSG00000130147), IL10RB(ENSG00000243646), RNF144B(ENSG00000137393), MXRA7(ENSG00000182534), F2R(ENSG00000181104), MCOLN1(ENSG00000090674), SLC16A14(ENSG00000163053), ARHGAP21(ENSG00000107863), CRK(ENSG00000167193), FFAR2(ENSG00000126262), BMPR1A(ENSG00000107779), ATRAID(ENSG00000138085), ABCG8(ENSG00000143921), ANK2(ENSG00000145362), ASPHD1(ENSG00000174939), PAM(ENSG00000145730), ABCC1(ENSG00000103222), NIPAL4(ENSG00000172548), APLNR(ENSG00000134817), MYORG(ENSG00000164976), UST(ENSG00000111962), RUFY3(ENSG00000018189), TMEM219(ENSG00000149932), AIF1L(ENSG00000126878), CYP4X1(ENSG00000186377), BCL2(ENSG00000171791), BBS1(ENSG00000174483), SLC13A5(ENSG00000141485), APLNR(ENSG00000134817), GPR37(ENSG00000170775), CYP2B6(ENSG00000197408), MADCAM1(ENSG00000099866), F2R(ENSG00000181104), SREBF1(ENSG00000072310), ABCC1(ENSG00000103222), DYSF(ENSG00000135636), TMEM52B(ENSG00000165685), ATP6V1D(ENSG00000100554), ATRAID(ENSG00000138085), CLIC4(ENSG00000169504), ST8SIA2(ENSG00000140557), TNFRSF10C(ENSG00000173535), SLC26A4(ENSG00000091137), FFAR2(ENSG00000126262), GNAZ(ENSG00000128266), ZDHHC2(ENSG00000104219), SLC30A7(ENSG00000162695), SFT2D2(ENSG00000213064), TMEM74B(ENSG00000125895), TNFSF14(ENSG00000125735), TTC17(ENSG00000052841), ACY3(ENSG00000132744), CHST11(ENSG00000171310), FIBCD1(ENSG00000130720), ZGRF1(ENSG00000138658), PIM1(ENSG00000137193), SRI(ENSG00000075142), SLC26A2(ENSG00000155850), TNFRSF11A(ENSG00000141655), AHCYL1(ENSG00000168710), SFXN5(ENSG00000144040), NDUFS8(ENSG00000110717), NTRK3(ENSG00000140538), PARD3(ENSG00000148498), PLPP1(ENSG00000067113), NOXA1(ENSG00000188747), RHOH(ENSG00000168421), PIGR(ENSG00000162896), TPCN1(ENSG00000186815), TULP3(ENSG00000078246), TJP3(ENSG00000105289), TCIRG1(ENSG00000110719), LMAN2(ENSG00000169223), PIGU(ENSG00000101464), RAP1GAP(ENSG00000076864), MFSD10(ENSG00000109736), SEL1L3(ENSG00000091490), SERP1(ENSG00000120742), SCARA3(ENSG00000168077), ITGA9(ENSG00000144668), NECAP1(ENSG00000089818), PLIN2(ENSG00000147872), ABCC3(ENSG00000108846), ABCB1(ENSG00000085563),
GPR183(ENSG00000169508), SLC24A3(ENSG00000185052), MACF1(ENSG00000127603), MGST2(ENSG00000085871), DENND5B(ENSG00000170456), MEGF8(ENSG00000105429), PNKD(ENSG00000127838), SERP2(ENSG00000151778), TNFAIP8L3(ENSG00000183578), SLC3A1(ENSG00000138079), VPS28(ENSG00000160948), ZDHHC8(ENSG00000099904), TMEM128(ENSG00000132406), TMEM64(ENSG00000180694), UBR4(ENSG00000127481), TSPAN31(ENSG00000135452), TNFRSF14(ENSG00000157873), TMEM201(ENSG00000188807), TSC2(ENSG00000103197), MCUR1(ENSG00000050393), SUSD6(ENSG00000100647), SERINC1(ENSG00000111897), RAB31(ENSG00000168461), TRPM4(ENSG00000130529), VTI1B(ENSG00000100568), TNFRSF10A(ENSG00000104689), TMEM164(ENSG00000157600), STK38L(ENSG00000211455), SLC25A23(ENSG00000125648), IMMT(ENSG00000132305), ADCK2(ENSG00000133597), ATG2B(ENSG00000066739), CCR10(ENSG00000184451), CHST3(ENSG00000122863), JAM3(ENSG00000166086), GPC4(ENSG00000076716), CX3CR1(ENSG00000168329), DPY19L4(ENSG00000156162), SLC48A1(ENSG00000211584), CLEC16A(ENSG00000038532), FCER1G(ENSG00000158869), DMXL2(ENSG00000104093), ARL8A(ENSG00000143862), CYSLTR1(ENSG00000173198), B3GNT5(ENSG00000176597), FAR2(ENSG00000064763), TESC(ENSG00000088992), CLN6(ENSG00000128973), CTNND1(ENSG00000198561), FXYD6(ENSG00000137726), ARL8B(ENSG00000134108), ELOVL1(ENSG00000066322), ANKRD13A(ENSG00000076513), CD1E(ENSG00000158488), SLC37A4(ENSG00000137700), HRH1(ENSG00000196639), GJA4(ENSG00000187513), GALNT16(ENSG00000100626), DENND5A(ENSG00000184014), SLC31A1(ENSG00000136868), ALG2(ENSG00000119523), ACKR3(ENSG00000144476), CD63(ENSG00000135404), CLDND1(ENSG00000080822), CYP20A1(ENSG00000119004), B3GNT6(ENSG00000198488), TMEM30B(ENSG00000182107), CMTM8(ENSG00000170293), AGPS(ENSG00000018510), BCL2L13(ENSG00000099968), CASD1(ENSG00000127995), ABCD3(ENSG00000117528), GRB14(ENSG00000115290), MPZL1(ENSG00000197965), NBAS(ENSG00000151779), SLC2A10(ENSG00000197496), APOOL(ENSG00000155008), HIGD1A(ENSG00000181061), MT-ND4(ENSG00000198886), POMGNT1(ENSG00000085998), ICA1(ENSG00000003147), MFSD2A(ENSG00000168389), SLC16A7(ENSG00000118596), PDLIM4(ENSG00000131435), GPR63(ENSG00000112218), CHRM3(ENSG00000133019), RNF144A(ENSG00000151692), PIGS(ENSG00000087111), PTPRB(ENSG00000127329), HLA-DRB1(ENSG00000196126), CLCN5(ENSG00000171365), CTXN2(ENSG00000233932), ABCA8(ENSG00000141338), AP3S1(ENSG00000177879), AP3S2(ENSG00000157823), CACNA1C(ENSG00000151067), CAVIN2(ENSG00000168497), GK(ENSG00000198814), ZDHHC2(ENSG00000104219), SFT2D2(ENSG00000213064), TMEM74B(ENSG00000125895), CHRM3(ENSG00000133019), ACER3(ENSG00000078124), CHST11(ENSG00000171310), SFXN5(ENSG00000144040), PARD3(ENSG00000148498), NOXA1(ENSG00000188747), TULP3(ENSG00000078246), TCIRG1(ENSG00000110719), PIGU(ENSG00000101464), RAP1GAP(ENSG00000076864), MFSD10(ENSG00000109736), SERP1(ENSG00000120742), ITGA9(ENSG00000144668), NECAP1(ENSG00000089818), PLIN2(ENSG00000147872), ABCC3(ENSG00000108846), GPR183(ENSG00000169508), MACF1(ENSG00000127603), MGST2(ENSG00000085871), MERTK(ENSG00000153208), PNKD(ENSG00000127838), SERP2(ENSG00000151778), VPS28(ENSG00000160948), TMEM260(ENSG00000070269), TMEM128(ENSG00000132406), UBR4(ENSG00000127481), TNFRSF14(ENSG00000157873), TSC2(ENSG00000103197), SUSD6(ENSG00000100647), TRPM4(ENSG00000130529), VTI1B(ENSG00000100568), TMEM164(ENSG00000157600), ADCK2(ENSG00000133597), CCR10(ENSG00000184451), CHST3(ENSG00000122863), GPC4(ENSG00000076716), DPY19L4(ENSG00000156162), CLEC16A(ENSG00000038532), DMXL2(ENSG00000104093), ARL8A(ENSG00000143862), CLCN5(ENSG00000171365), CYSLTR1(ENSG00000173198), FAR2(ENSG00000064763), TESC(ENSG00000088992), CTNND1(ENSG00000198561), FXYD6(ENSG00000137726), ARL8B(ENSG00000134108), ELOVL1(ENSG00000066322), ANKRD13A(ENSG00000076513), CTXN2(ENSG00000233932), EDNRB(ENSG00000136160), GJA4(ENSG00000187513), GALNT16(ENSG00000100626), ABCA8(ENSG00000141338), CLDND1(ENSG00000080822), TMEM63B(ENSG00000137216), TMEM30B(ENSG00000182107), COL13A1(ENSG00000197467), AGPS(ENSG00000018510), BCL2L13(ENSG00000099968), ABCD3(ENSG00000117528), MPZL1(ENSG00000197965), NBAS(ENSG00000151779), SLC2A10(ENSG00000197496), APOOL(ENSG00000155008), HIGD1A(ENSG00000181061), POMGNT1(ENSG00000085998), SLC2A11(ENSG00000133460),MFSD2A(ENSG00000168389), SLC30A7(ENSG00000162695), FIBCD1(ENSG00000130720), TNFRSF11A(ENSG00000141655), PDGFC(ENSG00000145431), PIGS(ENSG00000087111), RHOH(ENSG00000168421), TPCN1(ENSG00000186815), TJP3(ENSG00000105289), SEL1L3(ENSG00000091490), SCARA3(ENSG00000168077), DENND5B(ENSG00000170456), TMEM64(ENSG00000180694), TSPAN31(ENSG00000135452), TMEM201(ENSG00000188807), MCUR1(ENSG00000050393), SERINC1(ENSG00000111897), RAB31(ENSG00000168461), TNFRSF10A(ENSG00000104689), B3GNT5(ENSG00000176597), GLMP(ENSG00000198715), SLC37A4(ENSG00000137700), HRH1(ENSG00000196639), SLC31A1(ENSG00000136868), AP3S1(ENSG00000177879), AP3S2(ENSG00000157823), CMTM8(ENSG00000170293), CACNA1C(ENSG00000151067), CAVIN2(ENSG00000168497), MPP2(ENSG00000108852), HAS3(ENSG00000103044), MT-ND4(ENSG00000198886), PDLIM4(ENSG00000131435), GPR63(ENSG00000112218), RDX(ENSG00000137710), MERTK(ENSG00000153208), TMEM260(ENSG00000070269), EVA1A(ENSG00000115363), CDH2(ENSG00000170558), TMEM63B(ENSG00000137216), CYTH2(ENSG00000105443), SLC2A11(ENSG00000133460), ACER3(ENSG00000078124), PDGFC(ENSG00000145431), HAS3(ENSG00000103044), NAV3(ENSG00000067798), PDE9A(ENSG00000160191), UCHL1(ENSG00000154277), PLCD4(ENSG00000115556), TMPRSS2(ENSG00000184012), EDNRB(ENSG00000136160), COL13A1(ENSG00000197467), MPP2(ENSG00000108852), KCNK5(ENSG00000164626), GLMP(ENSG00000198715), ARFGAP3(ENSG00000242247), ENO2(ENSG00000111674), TMEM263(ENSG00000151135), SEMA5A(ENSG00000112902), SLC26A1(ENSG00000145217), ITGB1(ENSG00000150093), ADIPOR2(ENSG00000006831), UPK3BL2(ENSG00000284981), UPK3BL1(ENSG00000267368), SLC39A2(ENSG00000165794), EFHD1(ENSG00000115468), ABCG1(ENSG00000160179), ADGRA3(ENSG00000152990), GJA1(ENSG00000152661), ANKFY1(ENSG00000185722), BIK(ENSG00000100290), COL6A2(ENSG00000142173), VIPR2(ENSG00000106018), TRPV4(ENSG00000111199), TMEM43(ENSG00000170876), TMEM255B(ENSG00000184497), CAVIN1(ENSG00000177469), CDH11(ENSG00000140937), FCAR(ENSG00000186431), CAV1(ENSG00000105974), ANXA1(ENSG00000135046), VIPR2(ENSG00000106018), TRPV4(ENSG00000111199), TMEM263(ENSG00000151135), SEMA5A(ENSG00000112902), SLC26A1(ENSG00000145217), TMEM43(ENSG00000170876), ADIPOR2(ENSG00000006831), TMEM255B(ENSG00000184497), SLC39A2(ENSG00000165794), CLCA2(ENSG00000137975), ABCG1(ENSG00000160179), CAVIN1(ENSG00000177469), CDH11(ENSG00000140937), ADGRA3(ENSG00000152990), GJA1(ENSG00000152661), ANKFY1(ENSG00000185722), CAV1(ENSG00000105974), BIK(ENSG00000100290), ANXA1(ENSG00000135046), ZDHHC6(ENSG00000023041), ABHD17C(ENSG00000136379), GAA(ENSG00000171298), ZDHHC6(ENSG00000023041), KCNJ1(ENSG00000151704), ABHD17C(ENSG00000136379), CFC1(ENSG00000136698), SLC25A33(ENSG00000171612), RNF215(ENSG00000099999), MAST2(ENSG00000086015), XKR9(ENSG00000221947), VSTM5(ENSG00000214376), RNF103(ENSG00000239305), C2CD2L(ENSG00000172375), IGSF8(ENSG00000162729), PHKA1(ENSG00000067177),DOCK8(ENSG00000107099),IFFO1(ENSG00000010295),MARCKS(ENSG00000277443), SLC6A14(ENSG00000268104), RNF215(ENSG00000099999), MAST2(ENSG00000086015), VSTM5(ENSG00000214376), C2CD2L(ENSG00000172375), IGSF8(ENSG00000162729), PHKA1(ENSG00000067177), MARCKS(ENSG00000277443), SLC2A1(ENSG00000117394), SLC6A14(ENSG00000268104), SLC25A33(ENSG00000171612), XKR9(ENSG00000221947), HLA-
DPA1(ENSG00000231389), IFFO1(ENSG00000010295), AKAP12(ENSG00000131016), PPP1R3A(ENSG00000154415), TMEM245(ENSG00000106771), TEX2(ENSG00000136478), SCD5(ENSG00000145284), TSPAN33(ENSG00000158457), RRAD(ENSG00000166592), SNX1(ENSG00000028528), SMPD2(ENSG00000135587), NDUFAF5(ENSG00000101247), GNAI3(ENSG00000065135), MSRA(ENSG00000175806), MRC2(ENSG00000011028), MFN2(ENSG00000116688), UTRN(ENSG00000152818), ARHGEF12(ENSG00000196914), GNAI1(ENSG00000127955), HTR7(ENSG00000148680), FGFRL1(ENSG00000127418), GABARAPL1(ENSG00000139112), FMNL3(ENSG00000161791), DAG1(ENSG00000173402), CLN5(ENSG00000102805), ANO9(ENSG00000185101), DAPP1(ENSG00000070190), CLEC2B(ENSG00000110852), ICAM1(ENSG00000090339), TMEM245(ENSG00000106771), RTN3(ENSG00000133318), SNX1(ENSG00000028528), SMPD2(ENSG00000135587), GNAI3(ENSG00000065135), MRC2(ENSG00000011028), UTRN(ENSG00000152818), ARHGEF12(ENSG00000196914), GNAIl(ENSG00000127955), FGFRL1(ENSG00000127418), FMNL3(ENSG00000161791), DAG1(ENSG00000173402), CLN5(ENSG00000102805), ANO9(ENSG00000185101), DAPP1(ENSG00000070190), LRIG1(ENSG00000144749), ARMCX2(ENSG00000184867), TEX2(ENSG00000136478), SCD5(ENSG00000145284), TSPAN33(ENSG00000158457), NDUFAF5(ENSG00000101247), MFN2(ENSG00000116688), CLEC2B(ENSG00000110852), LRIG1(ENSG00000144749), ARMCX2(ENSG00000184867), LRRC24(ENSG00000254402), RTN3(ENSG00000133318), GPRIN1(ENSG00000169258), TMED6(ENSG00000157315), SCN4B(ENSG00000177098), DST(ENSG00000151914), CCND1(ENSG00000110092), SLC22A16(ENSG00000004809), SLC38A9(ENSG00000177058), SPIRE1(ENSG00000134278), SIRPB1(ENSG00000101307), PAG1(ENSG00000076641), PVR(ENSG00000073008), SLC25A36(ENSG00000114120), PCDHB15(ENSG00000113248), SNX7(ENSG00000162627), SLC11A1(ENSG00000018280), MPC2(ENSG00000143158), MGLL(ENSG00000074416), ITPR2(ENSG00000123104), MFSD11(ENSG00000092931), RINT1(ENSG00000135249), TMEM86A(ENSG00000151117), SLC38A5(ENSG00000017483), SUSD1(ENSG00000106868), REEP4(ENSG00000168476), GREB1(ENSG00000196208), KCNMB3(ENSG00000171121), DAB2(ENSG00000153071), HHAT(ENSG00000054392), CDK14(ENSG00000058091), APOBR(ENSG00000184730), ADCK5(ENSG00000173137), ACVRL1(ENSG00000139567), TMEM37(ENSG00000171227), ATP8B1(ENSG00000081923), ATG9A(ENSG00000198925), NFASC(ENSG00000163531), LFNG(ENSG00000106003), MRAP(ENSG00000170262), NDUFB9(ENSG00000147684), VAT1(ENSG00000108828), TTYH3(ENSG00000136295), TMED6(ENSG00000157315), SCN4B(ENSG00000177098), DST(ENSG00000151914), CCND1(ENSG00000110092), SPIRE1(ENSG00000134278), SIRPB1(ENSG00000101307), VAT1(ENSG00000108828), PCDHB15(ENSG00000113248), SNX7(ENSG00000162627), SLC11A1(ENSG00000018280), MGLL(ENSG00000074416), ITPR2(ENSG00000123104), TMEM86A(ENSG00000151117), SLC38A5(ENSG00000017483), REEP4(ENSG00000168476), GABBR1(ENSG00000204681), DAB2(ENSG00000153071), CDK14(ENSG00000058091), CLTCL1(ENSG00000070371), APOBR(ENSG00000184730), ADCK5(ENSG00000173137), ATP8B1(ENSG00000081923), NFASC(ENSG00000163531), LFNG(ENSG00000106003), NDUFB9(ENSG00000147684), SLC22A16(ENSG00000004809), SLC38A9(ENSG00000177058), MPC2(ENSG00000143158), RINT1(ENSG00000135249), GREB1(ENSG00000196208), KCNMB3(ENSG00000171121), MRAP(ENSG00000170262), KCNJ16(ENSG00000153822), NPHS1(ENSG00000161270), GABBR1(ENSG00000204681), CLTCL1(ENSG00000070371), ST8SIA5(ENSG00000101638), FXYD7(ENSG00000221946), FKBP11(ENSG00000134285), TSPAN12(ENSG00000106025), TBC1D9B(ENSG00000197226), TSPAN3(ENSG00000140391), SNX4(ENSG00000114520), SMAGP(ENSG00000170545), C2CD2(ENSG00000157617), PLPP2(ENSG00000141934), TREM2(ENSG00000095970), SEZ6L2(ENSG00000174938), TMEM94(ENSG00000177728), RHOA(ENSG00000067560), SMIM1(ENSG00000235169), PLXNA1(ENSG00000114554), SLC25A46(ENSG00000164209), RNF5(ENSG00000204308), MIEF2(ENSG00000177427), SRD5A3(ENSG00000128039), NAGPA(ENSG00000103174), NBEA(ENSG00000172915), TFPI(ENSG00000003436), RAB20(ENSG00000139832), RASD2(ENSG00000100302), RNF139(ENSG00000170881), SPCS3(ENSG00000129128), MGAT1(ENSG00000131446), KCNK1(ENSG00000135750), POMT2(ENSG00000009830), HEG1(ENSG00000173706), ILDR1(ENSG00000145103), LYPLA1(ENSG00000120992), TMEM159(ENSG00000011638), NUCB2(ENSG00000070081), PRIMA1(ENSG00000175785), VPS11(ENSG00000160695), SIGIRR(ENSG00000185187), VAMP1(ENSG00000139190), TMEM53(ENSG00000126106), SLC19A2(ENSG00000117479), SRPRB(ENSG00000144867), TECPR1(ENSG00000205356), SLC35E1(ENSG00000127526), VPS41(ENSG00000006715), FKTN(ENSG00000106692), KIFC3(ENSG00000140859), CYP2S1(ENSG00000167600), FAM171A1(ENSG00000148468), GCNT3(ENSG00000140297), B4GALT4(ENSG00000121578), CACNA1B(ENSG00000148408), DOK7(ENSG00000175920), ENTPD7(ENSG00000198018), EMC10(ENSG00000161671), FZD2(ENSG00000180340), HLA-DQB1(ENSG00000179344), CLMN(ENSG00000165959), COQ2(ENSG00000173085), CDIPT(ENSG00000103502), C6orf89(ENSG00000198663), CERS4(ENSG00000090661), DGAT1(ENSG00000185000), LIME1(ENSG00000203896), MALRD1(ENSG00000204740), ENTPD1(ENSG00000138185), CYP26B1(ENSG00000003137), FIBP(ENSG00000172500), ERBIN(ENSG00000112851), CHPF2(ENSG00000033100), SLC25A13(ENSG00000004864), CPM(ENSG00000135678), ATL2(ENSG00000119787), CLCA4(ENSG00000016602), COG8(EENNSSGG0000000000221732368107), ANKRD46(ENSG00000186106), ATRN(ENSG00000088812), ADGRL1(ENSG00000072071), BBS5(ENSG00000163093), CDH26(ENSG00000124215), UGT8(ENSG00000174607), PDZD3(ENSG00000172367), KCNA5(ENSG00000130037), GLCE(ENSG00000138604), SLC16A9(ENSG00000165449), KCNK10(ENSG00000100433), NOD2(ENSG00000167207), SLC16A4(ENSG00000168679), ITSN1(ENSG00000205726), LRP1(ENSG00000123384), CAPN2(ENSG00000162909), SMAGP(ENSG00000170545), C2CD2(ENSG00000157617), PLPP2(ENSG00000141934), PLXNA1(ENSG00000114554), RNF5(ENSG00000204308), MIEF2(ENSG00000177427), SRD5A3(ENSG00000128039), TFPI(ENSG00000003436), RNF139(ENSG00000170881), SPCS3(ENSG00000129128), MGAT1(ENSG00000131446), KCNK1(ENSG00000135750), HEG1(ENSG00000173706), VPS11(ENSG00000160695), VAMP1(ENSG00000139190), SLC19A2(ENSG00000117479), VPS41(ENSG00000006715), COX14(ENSG00000178449), C6orf89(ENSG00000198663), CERS4(ENSG00000090661), DGAT1(ENSG00000185000), MALRD1(ENSG00000204740), FIBP(ENSG00000172500), SLC25A13(ENSG00000004864), COG8(EENNSSGG0000000000221732368107), ADGRL1(ENSG00000072071), BBS5(ENSG00000163093), SLC16A9(ENSG00000165449), NOD2(ENSG00000167207), SLC16A4(ENSG00000168679), TSPAN12(ENSG00000106025), TBC1D9B(ENSG00000197226), SNX4(ENSG00000114520), NAGPA(ENSG00000103174), SUSD4(ENSG00000143502), NUCB2(ENSG00000070081), PRIMA1(ENSG00000175785), SIGIRR(ENSG00000185187), TMEM53(ENSG00000126106), TECPR1(ENSG00000205356), KIFC3(ENSG00000140859), FAM171A1(ENSG00000148468), B4GALT4(ENSG00000121578), CACNA1B(ENSG00000148408), DOK7(ENSG00000175920), EMC10(ENSG00000161671), FZD2(ENSG00000180340), SLC2A5(ENSG00000142583), ERBIN(ENSG00000112851), CHPF2(ENSG00000033100), ATL2(ENSG00000119787), ATRN(ENSG00000088812), KCNA5(ENSG00000130037), GLCE(ENSG00000138604), VDAC3(ENSG00000078668), MAGI2(ENSG00000187391), SNX19(ENSG00000120451), COX14(ENSG00000178449), SUSD4(ENSG00000143502), ATP6V1B1(ENSG00000116039), TRPV5(ENSG00000127412), SLC2A5(ENSG00000142583), NLGN1(ENSG00000169760), CCR4(ENSG00000183813), TMEM135(ENSG00000166575), STBD1(ENSG00000118804), PPP1R16B(ENSG00000101445), SDR16C5(ENSG00000170786), RDH13(ENSG00000160439), RAB28(ENSG00000157869), PRKN(ENSG00000185345), PLPPR2(ENSG00000105520), TMEM106C(ENSG00000134291), VAMP5(ENSG00000168899), TOMM40L(ENSG00000158882),
TMX2(ENSG00000213593), TSNARE1(ENSG00000171045), GRAP(ENSG00000154016), FAM162A(ENSG00000114023), AFG1L(ENSG00000135537), COMTD1(ENSG00000165644), PYCARD(ENSG00000103490), MAPK8IP1(ENSG00000121653), PCDH10(ENSG00000138650), CAV2(ENSG00000105971), MAP1LC3B(ENSG00000140941), TMEM135(ENSG00000166575), PPP1R16B(ENSG00000101445), SDR16C5(ENSG00000170786), RDH13(ENSG00000160439), RAB28(ENSG00000157869), VAMP5(ENSG00000168899), TMX2(ENSG00000213593), TSNARE1(ENSG00000171045), GRAP(ENSG00000154016), AFG1L(ENSG00000135537), CAV2(ENSG00000105971), COMTD1(ENSG00000165644), MAP1LC3B(ENSG00000140941), PLPPR2(ENSG00000105520), FAM162A(ENSG00000114023), PYCARD(ENSG00000103490), MAPK8IP1(ENSG00000121653), RMDN2(ENSG00000115841), PCDHB5(ENSG00000113209), PLOD1(ENSG00000083444), NUP54(ENSG00000138750), SLC22A1(ENSG00000175003), SDCBP2(ENSG00000125775), FGFR2(ENSG00000066468), GBA2(ENSG00000070610), GPR34(ENSG00000171659), C1orf210(ENSG00000253313), EPB41L2(ENSG00000079819), LBR(ENSG00000143815), DGKG(ENSG00000058866), MMP2(ENSG00000087245), PCDHB5(ENSG00000113209), SLC22A1(ENSG00000175003), SDCBP2(ENSG00000125775), FGFR2(ENSG00000066468), GBA2(ENSG00000070610), GPR34(ENSG00000171659), C1orf210(ENSG00000253313), RMDN2(ENSG00000115841), RETSAT(ENSG00000042445), NUP54(ENSG00000138750), GSG1L(ENSG00000169181), EPB41L2(ENSG00000079819), RETSAT(ENSG00000042445), ZC3H12A(ENSG00000163874), IKBIP(ENSG00000166130), GSG1L(ENSG00000169181)

### List of All Proteins in the List Above Separately Categorized by Applicable Use

(Ensembl version 92.38 ID for the symbols below can be found in the table above):
1. Cancer treated: melanoma
   a. Tissue protected: Colon TTYH3, AXL, VPS52, MS4A8, MAP1LC3A, CD36, FCAMR, MGAT4C, SCGN, SLC9A2, TM4SF18, SNTG2, PCSK5, ALPL, SELENBP1, STX1A, SNX24, SMIM24, SMURF1, NAAA, SLC27A2, SEC13, SLC41A2, SOCS7, QRFPR, RPH3AL, MPP7, RAC1, RNF145, SERINC3, ARHGAP5, NFAM1, HCRTR1, LDLR, GPR65, RAC3, MUC12, WSCD1, HTRA1, RAB27B, KLRG1, PARVB, DLC1, PLCB3, PCDHA4, RECK, MUC1, PSTPIP2, RAB3B, PSCA, KCNH8, SDC1, TMEM45A, TPSG1, TRPM7, SLC18A1, SPPL3, STYK1, TMEM134, MAPKAP1, IFI27, ITLN1, PTGDR, SGMS1, SYNGR4, ITGB6, MUC15, PEX10, OSBPL7, CD46, PROM2, PLPP7, MGAT4A, LRRN2, OBSCN, PROM1, UGT2B4, TMEM72, TMEM132A, VSIG10, SYT11, TMEM236, SLC7A7, TMEM143, VPS35L, ZP2, SYTL4, SYTL3, TMEM41A, TSPAN8, UNC13B, PLAUR, SYTL2, PRRG2, TPRA1, SLC46A2, OCLN, RNF170, SPHK1, SLC28A3, SCAMP5, PTAFR, SYP, PIGZ, PTPRN2, RSAD2, SLC39A14, SLC6A12, SLC6A15, RAB25, SLC20A2, SLC39A10, TGFBR1, STK16, TRAF4, SIGLEC14, MTG2, SPTLC3, SEC11C, MARK2, SCN7A, SLC15A3, PNLDC1, ARHGAP27, RNF186, TSPAN15, TRAF3IP3, VTCN1, TAS2R38, TCTN3, UBL3, TMEM39B, SLC30A10, CA2, CDHR5, CLDN4, ARL4D, ACVR1B, GGT1, DPEP1, GSDMB, GPBAR1, GHR, KCNJ8, SYK, FPR1, ADCY10, ACSL5, CD177, CLCA1, GLP2R, CADM4, DSC2, GNB5, ADGRF1, RASGRP2, CLDN5, EPHA4, HCAR3, DSG2, AQP8, CNTFR, CEACAM5, CHST6, CA4, DEPDC5, EPCAM, GNG11, LPAR6, CTDNEP1, CPTP, HSD17B2, MAGI1, ENTPD8, FPGS, MAN1C1, FADS3, CXCR1, FADS2, FER1L6, ANKRD13B, BEST2, BRI3, CDC42EP5, CLDN8, GAL3ST2, HTR4, B3GNT8, ELOVL7, B3GALT5, CDHR2, CLDN7, CLN3, CRB3, ACE2, SH3BP5, ATP9A, CNTNAP3, CDH17, C3AR1, ATP11C, KCNIP3, ATP1A2, CIB1, ADGRG6, ASIC5, CEACAM6, DCXR, AKAP10, ARL4C, ANO1, ANXA13, CHST14, ARHGEF4, ABHD12, C12orf66, CNPPD1, BLNK, ALG14, CEACAM3, HTR2B, B3GAT1, CASQ1, HHLA2, MAOA, CYP2C9, DIP2A, FGFR4, IBTK, SLC9A3R2, NCEH1, LRFN5, MYZAP, LHFPL2, KCNN3, MS4A18, MC4R, MS4A12, MCUB, EPPK1, MSTO1, SLC2A6, KIAA2013, NIPAL1, SLC16A10, IL17RA, ITGB4, LY9, LYN, GPA33, ALOX12, MUC13, OR10R2, MRC1, NECA P2, KLRC3, HCAR2, PRKCA, PSKH1, NDST2,
   b. Tissue protected: Lung TGM2, AXL, VSIG4, SLC30A4, VPS52, EFNB3, CD300C, SLC22A15, SELENBP1, ST6GAL1, SCEL, SMURF1, RNF145, SERINC3, NFAM1, AGER, PLLP, SLC18A2, RECK, MUC1, REEP2, MGAT4B, SCTR, ITGB6, OSBPL7, CD46, EHD2, OBSCN, PPL, CD84, TMEM80, PRRG2, OCLN, RNF170, RHOG, RSAD2, SLC6A15, RAB25, SLC39A10, TM4SF1, STEAP1, SCN7A, PNLDC1, TSPAN15, UBL3, CA2, CLDN4, ACVR1B, EHD1, GJB4, GPBAR1, KCNJ8, FPR1, CD247, CCR7, CYB5A, ACSL5, GNB5, CLDN5, EPHA4, DSG2, HCLS1, CEACAM5, AIFM2, LPAR6, CXCR1, FADS2, ANKRD13B, AIG1, APBB1IP, CLDN8, CLDN7, AQP4, CYB561D1, C3AR1, C ACNA2D2, ACE, CEACAM6, AQP3, CHST14, CADM1, HTR2B, CXCR3, MAOA, IBTK, NCEH1, LAMP3, MC4R, EHD3, MSTO1, NSMF, SLC34A2, PSKH1, LRRK2,
   c. Tissue protected: Skin TTYH3, SUN3, VPS52, CLEC10A, CD300C, CD47, MGAT4C, PERP, SNTG2, ALPL, TMPRSS11D, SLC6A1, SCEL, SMURF1, SEC13, SLC41A2, TMEM97, MPP7, RAC1, RNF145, ARHGAP5, NFAM1, SLC26A9, RAC3, RAB27B, MTMR2, MUC1, REEP2, KCNH8, GPIHBP1, SDC1, LTBR, TMEM45A, MGAT4B, TYROBP, STYK1, TMEM134, SLC10A6, MAPKAP1, IFI27, SGMS1, MUC15, OLFM2, PROM2, PLPP7, KRT1, PLA2G4E, LRRN2, DMPK, OBSCN, PPL, TMEM79, SLC7A7, YRDC, ZP2, SYTL3, TACSTD2, PRRG2, MSLN, OCLN, SPHK1, SLC28A3, S1PR5, PTAFR, RAB25, SLC20A2, SLC39A10, STK16, SLC19A1, MARK2, PNLDC1, TRAF3IP3, TAS2R38, TMEM39B, CLDN4, CYBA, GJB4, KCNJ8, SYK, FA2H, FPR1, DSP, ADCY10, CDH6, CD177, GLP2R, ANO7, FYN, DSC2, GPX8, CLDN5, EPHA4, HCAR3, DSG2, GPR39, CD248, CAPNS2, HCN1, DSC3, GSDMA, FGFBP1, CTDNEP1, FADS3, FADS2, B3GNT4, DSG1, ADGRF4, BRI3, ELOVL7, MS4A1, ASPRV1, CIB1, ASIC5, DSG3, DCXR, AQP3, CLEC2A, CNPPD1, BLNK, MAOA, IBTK, MYZAP, MC4R, MCUB, EPPK1, FGFR3, MSTO1, NIPAL1, ITGB4, ALOX12, OR10R2, HCAR2, PSKH1, SLC30A4, MS4A8, PI4KB, ULBP3, GPR65, RHBG, PLLP, DLC1, PLN, OSBPL7, CD46, ICAM3, SLC25A15, TMEM80, TMEM143, UNC13B, RHCG, TPRA1, SNTB1, SH2D3C, RNF170, RHO G, RSAD2, S100A8, RAB11FIP5, TM4SF1, TRAF4, MTG2, SEMA5B, ARHGAP27, TSPAN15, VTCN1, TCTN3, EHD1, GSDMC, CYB5A, EVI2A, FCGR2B, CNTFR, CEACAM5, GPAT2, CHST6, DNER, FGD2, CPTP, CXCR1, FNBP1, B3GNT8, BASP1, ATP9A, CYB561D1, CDH13, AKAP10, ANO1, CHST14, AP4E1, ALG14, HTR2B, NCEH1, LHFPL2, MUC21, EHD3, IL17RA, NECAP2, ,
   d. Tissue protected: Liver TTYH3, AXL, VPS52, UPK1B, MAP1LC3A, CLEC10A, CD300C, CREB3L3, FCAMR, TM4SF18, PCSK5, PI4KB, ALPL, SELENBP1, SLC6A4, SNX24, ST6GAL1, NAAA, SLC25A40, SLC27A2, SLCO1B3, SLC41A2, SLCO1B1, SLC25A43, SLC27A5, SERINC3, NFAM1, LDLR, GPR65, SLC18A2, RAB27B, MTMR2, PTGDR2, RECK, PDZK1, RDH16, LIN7A, SDC1, TMEM45A, MGAT4B, SPPL3, MAPKAP1, SGMS1, SYNGR4, PRRT2, CD46, OLFM2, NAT8, PNPLA3, LRRN2, SLC25A15, OBSCN, UGT2B4, TMEM132A, YIPF1, TFR2, VPS35L, UGT1A6, SYTL4, TNS2, UNC13B, ST7L, OCLN, RNF170, SPHK1, PTAFR, RSAD2, SLC39A14, SLC6A12, RAB25, TGFBR1, SLCO2B1, MARK2, TEX261, TSPAN15, TSPAN2, SLC30A10, CA2, CDHR5, ACVR1B, GGT1, GSDMB, GHR, KCNJ8, GLRB, CYP2D6, CYB5A, ACSL5, CD177, FYN, GAS2, CADM4, DSC2, SLC2A2, RASGRP2, CYP2A7, EPHA4, HCAR3, DSG2, FOLH1, CYP1A2, CNTFR, CYP3A4, CEACAM5, CYP4V2, DEPDC5, NINJ1, CYP2C8, DHCR24, CTDNEP1, HSD17B2, MAL2, ENTPD8, MAN1C1, CYP2C19, FADS3, FADS2, AQP9, AIG1, BRI3, CDHR2, CRB3, ASGR1, CYB561D1, ADRA2B, ABCB11, ATP1A2, CMKLR1, ASIC5, ACAD11, DCXR, ANO1, BACE2, CD320, ASGR2, PIK3AP1, CYP3A43, B3GAT1, CASQ1, INTS2, MAOA, CYP2C9, FGFR4, IBTK, MOXD1, SLC9A3R2, MYZAP, LHFPL2, MFAP3L, KCND1, MSTO1, GPR15, SLC2A6, NIPAL1, IL17RA, ITGB4, SLC2A8, ALOX12, OR10R2, ABCC2, HCAR2, DPP4, PSKH1, LRRK2, NDST2, NPC1L1,
   e. Tissue protected: Kidney (glomeruli) AXL, ZDHHC16, CD47, MGAT4C, SLC9A2, TM4SF18, SLC22A15, SLC45A1, SMURF1, RAC1, RNF145, PODXL, NFAM1, RAC3, PARVB, RECK, TPSG1, PECAM1, SCUBE1, PTGDR, SGMS1, PLA2R1, CD46, PROM2, PNPLA3, MGAT4A, MAP6, EHD2, MPP5, NPHS2, VPS35L, TSPAN7, TNS2, PRRG2, TPRA1, PTAFR, RSAD2, SLC6A12, THSD7A, PITPNM3, PTPRO, SCN7A, TSPAN15, ARL4D, EHD1, HYAL2, CDH6, FYN, GAS2, CD93, FAM107A, GNB5, ADGRF1, CD34, RASGRP2, CLDN5, EVI2A, DSG2, ENG, CD248, DOCK5, CR1, FGD2, FADS3, FADS2, FNBP1, B3GALT5, CLN3, CRB3, ACKR1, CDH13, B3GAT1, MAOA, SLC9A3R2, EHD3, MSTO1, DPP4, PSKH1,
   f. Tissue protected: Kidney (tubules) TTYH3, AXL, SUN3, TRPM3, VPS52, TBC1D3G, TREH, MS4A8, FCAMR, MGAT4C, SLC9A2, THY1, TM4SF18, SLC22A15, SELPLG, TMEM240, PCSK5, PI4KB, ALPL, SELENBP1, SLC6A4, SMIM24, SLC5A12, ST6GAL1, SLC12A1, SMURF1, NAAA, SLC25A40, SLC27A2, SLC4A10, SLC41A2, QRFPR, RPH3AL, SLC6A19, SLC22A11, S1PR2, MPP7, SERINC3, ARHGAP5, SLC28A2, NFAM1, HCRTR1, RDH11, LDLR, GPR65, AGER, RHBG, PLLP, WSCD1, VHL, HTRA1, RAB27B, PARVB, DLC1, PLCB3, PCDHA4, PDE2A, RECK, MUC1, RALB, PDZK1, PSCA, KCNH8, LIN7A, SDC1, LTBR, TMEM45A, MGAT4B, TRPM7, TMEM132E, SPPL3, STYK1, TMEM134, SCTR, MAPKAP1, IFI27, PTGDR, SGMS1, ITGB6, MUC15, NOS1, PEX10, PRRT2, OSBPL7, CD46, OLFM2, PROM2, NAT8, PNPLA3, PLPP7, MGAT4A, LRRN2, NTRK2, EHD2, MPP5, SLC25A15, OBSCN, PPL, PROM1, RFTN2, UGT2B4, SLC22A6, TMEM72, UGT3A1, TMEM132A, VSIG10, YIPF1, ATP6V0A4, SLC7A7, TMEM80, VPS35L, ZP2, UGT1A6, SYTL4, TMEM252, SYTL3, TMEM174, TMEM41A, TSPAN8, UNC13B, PLAUR, RHCG, SYTL2, PRRG2, TPRA1, FASLG, SLC46A2, SLC28A1, SNTB1, SIRT2, SLC22A2, SH2D3C, OCLN, RNF170, SPHK1, RHOG, SLC28A3, SCAMP5, PTAFR, SLC22A8, PIGZ, RSAD2, SLC39A14, SLC6A12, RAB25, RAB11FIP5, SLC20A2, SLC6A18, SLC13A2, TGFBR1, TMEM213, SLC5A1, STK16, TRAF4, SIGLEC14, SYT7, MTG2, SPTLC3, SLC4A4, SEC11C, MARK2, TMEM178B, TEX261, PKHD1, SLC35G2, PNLDC1, TSPAN2, VTCN1, TAS2R38, TSPAN16, UBL3, TMEM39B, CA2, CDHR5, CLDN4, ARL4D, ENPP6, EMC4, ACVR1B, GGT1, IYD, DPEP1, ENPP3, CUBN, EHD1, GSDMB, DLK1, GPBAR1, GHR, HYAL2, KCNJ8, GLRB, GSDMC, SYK, KIRREL2, CPNE6, CYB5A, JAKMIP1, CLTRN, ADCY10, ACSL5, CDH6, CD177, CPT1C, GAS2, CADM4, FAM107A, SLC2A2, GNB5, CLCNKB, ADGRG5, CALCR, CLDN5, CLCNKA, EPHA4, EVI2A, FAM169A, HCAR3, DSG2, GPR39, CASR, FOLH1, CNTFR, CD248, CHST6, CA4, CYP4V2, DEPDC5, EPCAM, GNG11, GLIPR1L2, LPXN, KIAA0319, DHCR24, LPAR6, CTDNEP1, LRFN4, CPTP, MAL2, MAGI1, FPGS, MAN1C1, FADS3, CXCR1, FADS2, AQP6, DSG1, AIG1, BRI3, CDC42EP5, CLDN8, NPR3, HTR4, CDC42SE1, CDHR2, CLDN7, CRB3, ADAM28, SH3BP5, ATP9A, SLC7A9, CLDN10, DUSP15, ATP11C, FXYD2, ACE, CMKLR1, CLDN2, CIB1, ASIC5, DCXR, AKAP10, ARL4C, ANO5, ANO1, ANXA13, AQP3, CHST14, ARHGEF4, AP4E1, C12orf66, BLNK, PIK3AP1, CYP3A43, CDH16, CADM1, HTR2B, B3GAT1, CASQ1, INTS2, MAOA, DIP2A, FGFR4, IBTK, LRFN2, MOXD1, NCEH1, MYZAP, LHFPL2, KCNJ10, KCNJ12, MFAP3L, MC4R, KCND1, MCUB, EHD3, EPPK1, MSTO1, MS4A4A, SLC2A6, KIAA2013, NIPAL1, SLC16A 10, IL17RA, FMO1, LRP2, ALOX12, IL23R, OR10R2, MRC1, GRIN2D, NECAP2, NSMF, ABCC2, MDGA2, HCAR2, KCTD8, PRKCA, DPP4, PSKH1, LRRK2, NDST2, NPC1L1,
   g. Tissue protected: Heart (cardiomyocytes) TGM2, TTYH3, AXL, SLC30A4, SLC27A6, SPAG4, UCP3, EFNB3, CD300C, CD36, CD47, MGAT4C, RASL10B, SLC22A15, PCSK5, PI4KB, SNX24, NAAA, SLC4A10, SEC13, SLC41A2, RAC1, RNF145, SERINC3, NFAM1, LDLR, RAC3, WSCD1, HTRA1, PARVB, POPDC2, RECK, PLN, GPIHBP1, INPP5D, MGAT4B, SGCG, SRL, MAPKAP1, SGMS1, ITGB6, PRRT2, OSBPL7, CD46, OLFM2, ART3, PLPP7, KLHL41, MGAT4A, LRRN2, DMPK, EHD2, SLC25A15, OBSCN, RFTN2, TMEM132A, YIPF1, TMEM236, SLC7A7, YRDC, TMEM143, TREML1, VPS35L, ZP2, TMEM41A, PRRG2, TPRA1, SNTB1, SIRT2, SH2D3C, RNF170, SPHK1, SCAMP5, PTAFR, RAB11FIP4, PIGZ, RSAD2, SLC39A14, SLC6A12, RYR1, SLAMF8, SLC20A2, SLC39A10, TM4SF1, TGFBR1, SLCO2B1, STK16, TRAF4, MTG2, MARK2, T MEM178B, SEMA5B, TEX261, SCN7A, SLC35G2, SLC15A3, TSPAN15, VTCN1, TAS2R38, TCTN3, UBL3, TMEM39B, EHD1, GSDMB, MMP27, FPR1, DSP, CDH6, CDH15, ADRA1A, CADM4, DSC2, GPX8, GNB5, ADGRF1, RASGRP2, CAVIN4, EPHA4, HCAR3, DSG2, GPR39, FCGR2B, CHRNA1, CNTFR, CD248, GPAT2, CHST6, FGD2, CYP4V2, DEPDC5, CTDNEP1, CPTP, LEPROTL1, MAN1C1, FADS3, CXCR1, FADS2, ANKRD13B, B3GNT4, AIG1, B3GNT8, ELOVL7, CDC42SE1, CDHR2, CLN3, ADAM28, CNTNAP3, CSF3R, C3AR1, ATP11C, ATP1A2, CMKLR1, CDH13, CIB1, ADGRG6, ADRB1, ASIC5, ACAD11, DCXR, AKAP10, ARL4C, ANO5, ANO1, ARHGEF4, AP4E1, BACE2, CNPPD1, BLNK, CASQ1, MAOA, IBTK, JPH1, NCEH1, MYZAP, LHFPL2, MFAP3L, GPR182, MC4R, KCND1, MCUB, EHD3, MSTO1, GPR15, SLC16A10, ITGB4, SLC2A8, ALOX12, OR10R2, MCEMP1, HCAR2, PRKCA, DES, PSKH1, LRRK2, NDST2,
   h. Tissue protected: Thyroid SLC30A4, SLC27A6, VPS52, CD47, SLC9A2, TMEM100, TM4SF18, SLC22A15, PCSK5, PI4KB, ALPL, SELENBP1, SLITRK4, SLC5A5, SNX24, ST6GAL1, SMURF1, NAAA, SEC13, QRFPR, MPP7, RNF145, SERINC3, ARHGAP5, NFAM1, KCNQ5, TPO, GPR65, WSCD1, VHL, GPIHBP1, LIN7A, INPP5D, MAPKAP1, IFI27, SGMS1, MUC15, OSBPL7, CD46, OLFM2, PSD3, PROM2, PLPP7, MGAT4A, LRRN2, NTRK2, SLC25A15, OBSCN, PPL, PROM1, PORCN, YIPF1, SLC7A7, YRDC, TMEM80, VPS35L, SYTL4, SYTL3, TMEM41A, UNC13B, PRRG2, TPRA1, FASLG, OCLN, RNF170, SPHK1, PTAFR, RSAD2, SLC39A14, RAB25, SLC20A2, SLC39A10, SLCO2B1, STK16, TRAF4, MTG2, MARK2, TEX261, PNLDC1, TSPAN15, VTCN1, TCTN3, UBL3, TMEM39B, C19orf18, CLDN4, ACVR1B, IYD, DUOX1, EHD1, GHR, KCNJ8, SYK, ADCY10, CDH6, FYN, CADM4, DSC2, GPX8, GNB5, CLCNKB, EPHX4, RASGRP2, CLDN5, CLCNKA, EPHA4, FCRL1, HCAR3, DSG2, FCGR2B, CNTFR, GPAT2, FGD2, HCN1, IPCEF1, DEPDC5, EPCAM, LPXN, LPAR6, CTDNEP1, MAGI1, CXCR1, FADS2, FER1L6, ANKRD13B, FNBP1, ADD2, CLDN8, CLDN7, CRB3, BASP1, AAK1, ATP9A, AQP4, C3AR1, CMKLR1, CRHR1, CIB1, DCXR, AKAP10, ARL4C, CHST14, ARHGEF4, AP4E1, CD320, C12orf66, BLNK, CDH16, CDH10, CEACAM3, CADM1, INTS2, MAOA, IBTK, NCEH1, MYZAP, LHFPL2, LRP1B, MC4R, EPPK1, MSTO1, GPR15, SLC2A6, IL17RA, ITGB4, LYN, OR10R2, GRIN2D, NECAP2, HCAR2, PRKCA, PSKH1, NDST2,
   i. Tissue protected: Pancreatic endocrine cells (Islets) SLC30A4, VPS52, MAP1LC3A, CD47, SCGN, PI4KB, ALPL, STX1A, NAAA, RAB3A, SEC13, RTN1, QRFPR, RPH3AL, RNF145, SCG3, ARHGAP5, NFAM1, HCRTR1, POMGNT2, WSCD1, VHL, PTGDR2, RAB3B, SPPL3, MAPKAP1, SGMS1, ITGB6, PTPRN, CD46, OLFM2, MGAT4A, LRRN2, OBSCN, NECAB2, SLC17A6, SV2A, YRDC, SYTL4, TSPAN7, TNS2, UNC13B, PLAUR, SLC30A8, PRRG2, TPRA1, FASLG, RNF170, SCAMP5, SYP, PTPRN2, RSAD2, RAB25, SLCO2B1, STK16, SIGLEC14, SYT7, TEX261, MAPK10, SLC35G2, TSPAN2, TMEM39B, EMC4, DLK1, DGCR2, CLTRN, ADCY10, CDH6, GNB5, GNAO1, CLDN5, FAM169A, DSG2, CASR, AQP8, CNTFR, CYP4V2, EPCAM, LPAR6, CTDNEP1, MAGI1, CXCR1, FADS2, CLDN7, ATP9A, CLU, C3AR1, FXYD2, AMPH, ATP1A2, CRHR1, ASIC5, ARL4C, ARHGEF4, CADM1, GAD2, ALOX5, MAOA, FGFR4, IBTK, MC4R, KCND1, MSTO1, KIAA2013, FMO1, OR10R2, MDGA2, PRKCA, DPP4, PSKH1,
   j. Tissue protected: Pancreatic exocrine cells AXL, VPS52, CLEC10A, CD300C, RASL10B, TBC1D3D, TMEM100, TM4SF18, SELPLG, PCSK5, PI4KB, ALPL, ST6GAL1, SMURF1, NAAA, SLC27A2, SLC4A10, SEC13, SLC41A2, RPH3AL, TMEM97, RNF145, SEC16B, NFAM1, POMGNT2, PLLP, WSCD1, VHL, RAB27B, PCDHA4, MUC1, PSCA, KCNH8, SDC1, TMEM132E, STYK1, SCTR, SCUBE1, IFI27, SGMS1, ITGB6, MUC15, CD46, OLFM2, PROM2, PLPP7, MGAT4A, LRRN2, OBSCN, PPL, PROM1, TMEM72, UGT3A1, VSIG10, YRDC, TMEM80, SYTL4, UNC13D, TRAT1, TMEM41A, TSPAN8, UNC13B, PLAUR, TBC1D3E, SYTL2, TACSTD2, TPRA1, FASLG, SNTB1, OCLN, RNF170, SPHK1, SYCN, SLC28A3, SCAMP5, RSAD2, SLC39A14, RAB25, RAB11FIP5, SLC20A2, SLC39A10, TM4SF1, SLCO2B1, STK16, TRAF4, SYT7, MTG2, SPTLC3, SLC4A4, SEC11C, SLC19A1, MARK2, TMEM178B, PKHD1, SCN7A, PNLDC1, ARHGAP27, TSPAN15, TRAF3IP3, TAS2R38, TCTN3, TMEM39B, CA2, CLDN4, ARL4D, ACVR1B, GGT1, DPEP1, CUZD1, GHR, KCNJ8, GLRB, GP2, SYK, KIRREL2, FA2H, CYB5A, DSP, ADCY10, ACSL5, CDH6, CYSTM1, DSC2, ADGRF1, RASGRP2, CLDN5, EVI2A, DSG2, FCGR2B, AQP8, CNTFR, CD248, GPAT2, CHST6, FGD2, CA4, CYP4V2, EPCAM, LPAR6, CTDNEP1, MAGI1, FPGS, LEPROTL1, MAN1C1, FADS3, FADS2, BRI3, CLDN8, HTR4, CDH9, ELOVL7, CDC42SE1, CLDN7, CLN3, CRB3, ADAM28, ACE2, CNTNAP3, CFTR, CLDN10, ATP11C, FXYD2, CMKLR1, CIB1, CHST4, AKAP10, ARL4C, CHST14, AP4E1, C12orf66, PIK3AP1, ALG14, CADM1, HTR2B, CASQ1, MAOA, AQP12A, AQP12B, FGFR4, IBTK, MOXD1, NCEH1, LRFN5, MYZAP, MC4R, MCUB, EPPK1, MSTO1, SLC2A6, KIAA2013, SLC16A10, IL17RA, LYN, OR10R2, PRKCA, PSKH1, LRRK2, NDST2,
2. Cancer treated: lung cancer
   a. Tissue protected: Colon AXL, TMEM158, UGT2A3, MS4A8, MAP1LC3A, CACNB2, CORO7, ABCD4, CD36, FCAMR, GABARAPL2, MGAT4C, SCGN, TM4SF18, UBE2J1, SNTG2, SYT13, PCSK5, PLSCR4, STX1A, SMIM24, SPCS1, TNFSF12, NAAA, SLC27A2, HCRTR1, BVES, P2RX6, PLEKHA3, MUC12, TVP23B, WHAMM, PTPRD, KLRG1, DLC1, MED28, PLCB3, RECK, PTPRT, RAB3B, PSCA, KCNH8, MRGPRF, TMEM45A, TPSG1, TRPM7, SLC18A1, SPATA9, SLCO4A1, STYK1, SLC9A3, ITLN1, PTGDR, ADCYAP1R1, SYNGR4, PEX10, MRGPRX2, PCDH11X, PALM, PLPP7, VEPH1, PRKAR2B, CNEP1R1, SORD, GOLGA2, OBSCN, AATK, SPRED1, UGT2B4, SLC26A3, TMEM72, TMEM132A, UGT2B17, VSIG10, SYT11, SLC7A7, SERAC1, TVP23C, UGT2B28, ZP2, VAC14, QTRT1, TSPAN13, TMEM253, TBCD, UGT2B15, ARHGAP17, SEZ6, C2orf88, GOPC, SPHK1, SCAMP5, PTPRN2, SLC39A14, SLC6A12, SNX6, PHACTR2, RAB29, NAPG, SEC11C, PHLPP2, PRRT3, SLC35B1, NAPB, TRAF3IP3, TMEM154, TMEM171, TCTN3, TMEM39B, TMEM30A, CA2, GGT1, DPEP1, CLSTN1, FCER1A, IL9R, SLC2A12, CLCA1, GLP2R, CEACAM1, FPR2, CADM4, FKBP2, GRK5, CHDH, GNB5, FAM126B, HTR1A, GLT8D2, ENOX2, GATM, FLNC, GCHFR, AADAC, AQP8, DIAPH1, ADGRE1, CHODL, CA12, CA4, DPP10, DGKQ, GBP2, LRRC37B, CPTP, HSD17B2, MAGI1, ENTPD8, MAN1C1, AP3M1, FADS3, ABHD16A, ANKRD27, CXCR1, ANO8, AQP1, BEST2, BRI3, CDC42EP5, MPP1, GAL3ST2, CDC42EP1, UGCG, ELOVL7, CDHR2, CLN3, ACE2, SH3BP5, CNTNAP3, ENPEP, CLDN11, KCNIP3, ARRB1, CADPS, C5AR2, ARRDC1, ADGRG6, ASIC5, AKAP10, ANXA13, AP1S3, EPB41L3, ABHD12, ADGRV1, CNPPD1, EBP, BLNK, HSD3B7, ALG14, B3GAT1, CASQ1, CYP2C9, DIP2A, IBTK, ABCC5, SMPD4, HMOX1, LRFN5, SLC16A6, MYZAP, LHFPL2, NDUFS1, MS4A12, GFRA2, MCUB, GNG12, GLRA2, MAP2K1, SLC2A6, KIAA2013, NIPA2, NIPAL1, IGFLR1, ITGB4, GAL3ST1, DPY19L1, RPS6KC1, ALOX12, OR10R2, MRC1, KLRC3, PRKCA, PSKH1,
   b. Tissue protected: Lung AXL, VSIG4, SLC30A4, EFNB3, CACNB2, CD300C, TRIM13, UBE2J1, PLSCR4, SLC25A17, TGFBR3, SEMA4F, NUS1, BVES, AGER, TVP23B, SLC18A2, WHAMM, MED28, RECK, PTPRT, REEP2, PTPRG, SGIP1, MRGPRX2, PALM, VEPH1, KIRREL3, EHD2, OBSCN, CD84, SERAC1, TVP23C, QTRT1, TBC1D3, P2RY2, SEZ6, GOPC, RHOG, PSD4, PHACTR2, NAPG, RYK, PHLPP2, SIGLEC9, CA2, EHD1, GJB4, EHD4, CD247, CCR7, SLC44A2, AOC3, FKBP2, GRK5, GNB5, GLT8D2, AADAC, GBP2, AIFM2, FES, L1CAM, AP3M1, ANKRD27, CXCR1, AQP1, APBB1IP, CDC42EP1, ELMO2, ENPEP, CACNA2D2, ACE, ARRB1, BSN, BDKRB2, CXCR3, IBTK, EHD3, NIPA2, LDLRAD2, PI4KA, PSKH1, LRRK2,
   c. Tissue protected: Skin SUN3, CACNB2, CLEC10A, CD300C, ABCD4, GABARAPL2, MGAT4C, SOX10, SNTG2, SLC25A17, TGFBR3, SPCS1, TNFSF12, SLC6A1, TMEM97, NEGR1, NUS1, REEP2, KCNH8, GPIHBP1, TMEM45A, TYROBP, STYK1, SLC9A3, LANCL2, PALM, PLPP7, LAX1, VEPH1, SORD, DMPK, GOLGA2, OBSCN, AATK, TRPM2, RMC1, SLC7A7, SERAC1, YRDC, ZP2, VAC14, TBCD, UGT2B15, P2RY2, ARHGAP17, C2orf88, GOPC, SPHK1, PHACTR2, RAB29, RYK, SLC19A1, PRRT3, NAPB, SIGLEC9, TRAF3IP3, TMEM171, TMEM39B, TMEM30A, CYBA, GJB4, DSP, GLP2R, ANO7, CACNA2D1, FYN, FPR2, GPX8, FKBP2, GRK5, FAM126B, GLT8D2, ENOX2, GATM, AADAC, CD99, CD248, CAPNS2, CA12, HCN1, MALL, DGKQ, GBP2, GSDMA, LRRC37B, L1CAM, AP3M1, FADS3, ANKRD27, B3GNT4, BRI3, CDC42EP1, ELOVL7, CD81, BST1, ARRB1, CD1D, ASPRV1, ASIC5, DSG3, ADGRV1, CNPPD1, BLNK, HSD3B7, IBTK, ABCC5, MYZAP, MCUB, GNG12, MAP2K1, NIPA2, NIPAL1, ITGB4, LDLRAD2, RPS6KC1, ALOX12, CHL1, PI4KA, OR10R2, NCS1, PSKH1, TCTN2, SLC30A4, MS4A8, CORO7, TRIM13, UBE2J1, SYT13, PI4KB, ULBP3, TYR, WIPI2, DLC1, MPP6, PLN, PTPRT, SLCO4A1, KCNQ4, SGIP1, PRKAR2B, SLC25A15, SPRED1, QTRT1, TSPAN13, TBC1D3, DCT, ZC4H2, RHOG, RAB11FIP5, NAPG, PMEL, SEMA5B, PHLPP2, TCTN3, DTL, CNTN3, CLSTN1, EHD1, FCER1A, EHD4, ADCY7, EVI2A, TM7SF2, CYB561A3, FCGR2B, DIAPH1, FAM210A, ADGRE1, GPAT2, CD82, DNER, CPTP, CXCR1, PROCR, FNBP1, UGCG, ELMO2, CDH13, AKAP10, CPNE2, BDKRB2, ALG14, ADGRA1, LHFPL2, MUC21, NDUFS1, GFRA2, EHD3, ,
   d. Tissue protected: Liver VNN1, AXL, MAP1LC3A, CACNB2, CLEC10A, CD300C, CREB3L3, FCAMR, G ABARAPL2, TRIM13, TM4SF18, UBE2J1, SYT13, PCSK5, SLC25A17, PI4KB, SVEP1, SPCS1, NAAA, SLC27A2, SLCO1B3, SLCO1B1, SEMA4F, SLC27A5, NEGR1, PLEKHA3, SLC18A2, PTPRD, KSR2, PTGDR2, RECK, PTPRT, LIN7A, TMEM45A, KCNQ4, SYNGR4, PRRT2, MRGPRX2, NAT8, PNPLA3, VEPH1, KIRREL3, SORD, GOLGA2, MME, SLC25A15, OBSCN, AATK, SPRED1, UGT2B4, TMEM132A, YIPF1, TFR2, SERAC1, TNS2, TBCD, ZDHHC13, UGT2B15, ARHGAP17, SLC25A25, ST7L, C2orf88, GOPC, SPHK1, SLC39A14, SLC6A12, SLCO2B1, RAB29, NAPG, TEX261, NAPB, SIGLEC9, TMEM171, TMEM30A, CA2, GGT1, FCER1A, GLRB, CYP2D6, CACNA2D1, CEACAM1, FYN, FPR2, GAS2, CADM4, SLC2A2, CHDH, HTR1A, CREB3L2, CYP2A7, GLT8D2, HEPACAM, GATM, FOLH1, CYP1A2, GCHFR, AADAC, CYP3A4, DIAPH1, FAM210A, FMO3, MALL, GBP2, ABCB4, NINJ1, CYP2C8, LRRC37B, FES, KMO, HSD17B2, ENTPD8, MAN1C1, AP3M1, CYP2C19, FADS3, ANKRD27, BCL2L10, AQP9, BRI3, UGCG, CDHR2, ENPEP, ASGR1, ARRB1, ABCB11, CMKLR1, CTSL, ARRDC1, ASIC5, HSD11B1, ADGRV1, CD320, EBP, ASGR2, PIK3AP1, CYP3A43, HSD3B7, B3GAT1, CASQ1, INTS2, CYP2C9, IBTK, SMPD4, SLC16A6, MYZAP, LHFPL2, MFAP3L, LIMS2, NDUFS1, GPR15, SLC2A6, NIPAL1, IGFLR1, ITGB4, LDLRAD2, DPY19L1, SLC2A8, RPS6KC1, ALOX12, PI4KA, OR10R2, ABCC2, PSKH1, LRRK2, SLC10A1, NPC1L1,
   e. Tissue protected: Kidney (glomeruli) AXL, ZDHHC16, ZAP70, CACNB2, CORO7, MGAT4C, TM4SF18, UBE2J1, KDR, MAPT, SYT13, SLC25A17, TGFBR3, TNFSF12, SLC45A1, PODXL, KIRREL1, P2RX6, PLEKHA3, TVP23B, WHAMM, KSR2, RECK, PTPRT, PTPRG, MRGPRF, SLC34A1, TPSG1, PECAM1, SCUBE1, PTGDR, ADCYAP1R1, PLA2R1, PTH2R, PNPLA3, PALM, VEPH1, KIRREL3, PRKAR2B, EHD2, GOLGA2, MPP5, MME, NKD1, NPHS2, RMC1, SERAC1, TVP23C, VAC14, TSPAN7, TTC7B, TNS2, ARHGAP17, C2orf88, STK10, GOPC, SNCA, PSD4, SLC6A12, SNX6, PHACTR2, THSD7A, PEAR1, NAPG, RYK, PITPNM3, SLC35B1, RTL1, NAPB, TMEM171, CLSTN1, EHD1, HYAL2, EHD4, CEACAM1, FYN, GAS2, CD93, FAM107A, GNB5, CD34, FAM126B, HTR1A, GLT8D2, EVI2A, ENG, CD99, CD248, FAM210A, ADGRE1, CR1, GBP2, MAS1, ADGRL4, FADS3, ANKRD27, ANO8,AQP1, FNBP1, CDC42EP1, CLN3, ELMO2, ENPEP, ARRB1, CHRM1, BSN, CDH13, B3GAT1, SLC43A2, LIMS2, ITGA8, EHD3, GNG12, MLIP, NIPA2, LDLRAD2, PI4KA, PSKH1, PDGFRA,
   f. Tissue protected: Kidney (tubules) VNN1, AXL, SUN3, TRPM3, TBC1D3G, TREH, MS4A8, CACNB2, CORO7, ABCD4, FCAMR, GABARAPL2, MGAT4C, THY1, TM4SF18, UBE2J1, SELPLG, MAPT, TMEM240, PCSK5, PI4KB, SVEP1, SMIM24, SLC5A12, SLC5A2, SPCS1, TNFSF12, STIMATE, SLC12A1, TLN2, NAAA, PCDHB2, SLC27A2, SCNN1D, SLC6A19, SEMA4F, S1PR2, SLC34A3, SLC28A2, HCRTR1, RDH11, NEGR1, PTH1R, KIRREL1, P2RX6, AGER, TVP23B, WIPI2, WHAMM, PTPRD, DLC1, PLCB3, MPP6, PDE2A, RECK, PTPRT, RALB, PSCA, KCNH8, LIN7A, MRGPRF, TMEM45A, SLC34A1, TRPM7, PRICKLE1, SLCO4A1, STYK1, KCNQ4, SLC9A3, PTGDR, ADCYAP1R1, SGIP1, NOS1, PEX10, PRRT2, NAT8, PNPLA3, PALM, PLPP7, VEPH1, KIRREL3, CNEP1R1, NTRK2, SORD, EHD2, GOLGA2, MPP5, MME, SLC25A15, OBSCN, AATK, RFTN2, SPRED1, UGT2B4, SLC22A6, TMEM72, TRPM2, UGT3A1, XPNPEP2, TMEM132A, VSIG10, YIPF1, ATP6V0A4, SLC7A7, SERAC1, TVP23C, ZP2, VAC14, QTRT1, TMEM252, TSPAN13, TMEM174, TTC7B, TBC1D3, TBCD, ZDHHC13, ARHGAP17, SLC28A1, SIRT2, SLC22A2, C2orf88, GOPC, SPHK1, RHOG, SCAMP5, SLC22A8, SLC13A3, SLC39A14, SLC6A12, SNX6, PHACTR2, RAB11FIP5, SLC6A18, SLC13A2, TMEM213, SLC5A1, SYT7, RAB29, PEAR1, SLC4A4, NAPG, RYK, SEC11C, TEX261, PKHD1, PRRT3, SLC35G2, OPRD1, NAPB, TMEM171, TSPAN16, TMEM39B, TMEM30A, CA2, ENPP6, EMC4, GGT1, IYD, DPEP1, ENPP3, CUBN, CLSTN2, CLSTN1, EHD1, FCER1A, HYAL2, GLRB, KIRREL2, EHD4, CKMT2, CLTRN, SLC44A2, ADCY7, FAM151A, CPT1C, GAS2, CADM4, FAM107A, FNDC5, FKBP2, GRK5, SLC2A2, CHDH, DOCK2, GNB5, CLCNKB, FAM126B, HTR1A, ADGRG5, CLCNKA, CREB3L2, GLT8D2, ENOX2, EVI2A, FAM169A, TM7SF2, GATM, CASR, FOLH1, GCHFR, CD248, DIAPH1, FAM210A, ADGRE1, FRRS1L, CA12, CA4, FMO3, GLIPR1L2, LPXN, DGKQ, GBP2, KIAA0319, LRRC37B, FES, KMO, LRFN4, CPTP, L1CAM, MAGI1, MAN1C1, AP3M1, EPHB1, FADS3, ABHD16A, ANKRD27, BSND, BCL2L10, CXCR1, ANO8,AQP6, AQP1, BRI3, CDC42EP5, NPR3, MPP1, UGCG, CDHR2, SH3BP5, BST1, ENPEP, SLC7A9, FXYD2, ACE, ARRB1, CATIP, CMKLR1, BSN, C5AR2, CPT1B, COX7B, CTSL, ARRDC1, ASIC5, AKAP10, ANXA13, CPNE2, AP1S3, EPB41L3, ADGRV1, EBP, BLNK, PIK3AP1, C5AR1, CYP3A43, HSD3B7, CDH16, B3GAT1, CASQ1, INTS2, DIP2A, IBTK, ABCC5, SMPD4, HMOX1, LRFN2, SLC43A2, SLC16A6, MYZAP, LHFPL2, KCNJ10, KCNJ12, MFAP3L, NDUFS1, GFRA2, MCUB, EHD3, GNG12, MLIP, MAP2K1, SLC2A6, KIAA2013, NIPA2, NIPAL1, IGFLR1, FAM234B, FMO1, GAL3ST1, LDLRAD2, DPY19L1, RPS6KC1, LRP2, ALOX12, MFSD1, CHL1, PI4KA, IL23R, OR10R2, MRC1, GRIN2D, ABCC2, KCTD8, PRKCA, PSKH1, LRRK2, NPC1L1,
   g. Tissue protected: Heart (cardiomyocytes) AXL, TCTN2, SLC30A4, EFNB3, CAP2, CACNB2, CD300C, CD36, GABARAPL2, MGAT4C, RASL10B, MAPT, SYT13, PCSK5, SLC25A17, TGFBR3, PI4KB, SVEP1, SPCS1, TNFSF12, TLN2, NAAA, SGCB, NEGR1, NUS1, BVES, P2RX6, PLEKHA3, TVP23B, WHAMM, PTPRD, POPDC2, MPP6, P2RY6, RECK, PLN, GPIHBP1, MRGPRF, SHISA4, INPP5D, FLT1, SPATA9, SGCG, SLCO4A1, SRL, KCNQ4, SLC9A3, SGIP1, PRRT2, LANCL2, ART3, PLPP7, VEPH1, CNEP1R1, DMPK, EHD2, GOLGA2, SLC25A15, OBSCN, AATK, RFTN2, SPRED1, SIPA1, TMEM132A, YIPF1, SLC7A7, SERAC1, YRDC, TVP23C, TREML1, ZP2, VAC14, QTRT1, TSPAN13, TTC7B, TBC1D3, TBCD, ZDHHC13, ZC4H2, ARHGAP17, SLC25A25, SIRT2, C2orf88, GOPC, SPHK1, SCAMP5, RAB11FIP4, SLC39A14, SLC6A12, RYR1, SLAMF8, SNX6, PHACTR2, SLCO2B1, RAB29, PEAR1, NAPG, SEMA5B, TEX261, SLC35B1, SLC35G2, NAPB, TMEM171, TCTN3, TMEM39B, TMEM30A, CNTN3, EHD1, FCER1A, MMP27, EHD4, CKMT2, DSP, ADCY7, CACNA2D1, CDH15, ADRA1A, CADM4, GPX8, FNDC5, GRK5, CHDH, GNB5, FAM126B, HTR1A, CAVIN4, CREB3L2, SLC2A4, GLT8D2, TM7SF2, HHATL, CYB561A3, FCGR2B, FLNC, CHRNA1, CD248, GPAT2, MALL, DGKQ, GBP2, CAMK2B, LRRC37B, FES, MBOAT7, CPTP, L1CAM, LEPROTL1, MAN1C1, ADGRL4, AP3M1, FADS3, ABHD16A, ANKRD27, CXCR1, FCGRT, ANO8, B3GNT4, UGCG, DMD, ELOVL7, CD81, CDHR2, CLN3, CNTNAP3, BST1, CSF3R, CMKLR1, BSN, CADPS, CPT1B, COX7B, CDH13, CD1D, ARRDC1, AKAP6, ADGRG6, ADRB1, ASIC5, AKAP10, CPNE2, AP1S3, CNPPD1, BLNK, HSD3B7, CASQ1, IBTK, JPH1, ABCC5, SMPD4, SLC16A6, MYZAP, LHFPL2, MFAP3L, NCAM1, GPR182, LIMS2, NDUFS1, MCUB, EHD3, GPR15, MLIP, MAP2K1, IGFLR1, ITGB4, GAL3ST1, LDLRAD2, DPY19L1, SLC2A8, IL18RAP, RPS6KC1, ALOX12, PI4KA, OR10R2, MCEMP1, PRKCA, DES, PSKH1, LRRK2,
   h. Tissue protected: Thyroid TMEM158, TCTN2, SLC30A4, CACNB2, GABARAPL2, TMEM100, TRIM13, TM4SF18, UBE2J1, VPS33B, SYT13, PCSK5, PLSCR4, SLC25A17, TGFBR3, PI4KB, SVEP1, SLITRK4, SPCS1, TNFSF12, TLN2, NAAA, PCDHB2, SCNN1D, SEMA4F, NEGR1, NUS1, BVES, PLEKHA3, TVP23B, WIPI2, WHAMM, MED28, MPP6, GPIHBP1, LIN7A, INPP5D, PRICKLE1, SLCO4A1, KCNQ4, LANCL2, PSD3, PALM, PLPP7, VEPH1, CNEP1R1, NTRK2, SORD, GOLGA2, SLC25A15, OBSCN, AATK, SPRED1, TRPM2, YIPF1, SLC7A7, SERAC1, YRDC, TVP23C, VAC14, QTRT1, TSPAN13, TTC7B, TBC1D3, TBCD, ZC4H2, ARHGAP17, RTP5, SLC25A25, GOPC, SPHK1, SLC39A14, PHACTR2, RNFT2, SLCO2B1, RAB29, NAPG, RYK, TEX261, PRRT3, SIGLEC9, TMEM171, TCTN3, TMEM39B, C19orf18, TMEM30A, IYD, CNTN3, DUOX1, CLSTN1, EHD1, FCER1A, EHD4, FYN, FPR2, CADM4, GPX8, FKBP2, GNB5, CLCNKB, FAM126B, HTR1A, CLCNKA, CREB3L2, GLT8D2, GATM, FCGR2B, AADAC, CD99, DIAPH1, FAM210A, ADGRE1, GPAT2, HCN1, FMO3, DPP10, MALL, LPXN, DGKQ, CAMK2B, FES, MAGI1, ADGRL4, AP3M1, EPHB1, ANKRD27, CXCR1, ANO8,PROCR, FNBP1, CDC42EP1, UGCG, ELMO2, AAK1, ENPEP, ARRB1, CMKLR1, CRHR1, ARRDC1, AKAP10, CPNE2, AP1S3, ADGRV1, CD320, BLNK, C5AR1, HSD3B7, CDH16, INTS2, AP3B2, IBTK, SMPD4, MTNR1B, MYZAP, LHFPL2, NCAM1, NDUFS1, GFRA2, GNG12, GPR15, MAP2K1, SLC2A6, NIPA2, IGFLR1, ITGB4, FAM234B, GAL3ST1, MFSD1, PI4KA, OR10R2, GRIN2D, NCS1, PRKCA, PSKH1,
   i. Tissue protected: Pancreatic endocrine cells (Islets) SLC30A4, MAP1LC3A, CACNB2, ABCD4, SCGN, UBE2J1, MAPT, SYT13, PLSCR4, SLC25A17, TGFBR3, PI4KB, SVEP1, STX1A, NAAA, RAB3A, SCG3, HCRTR1, NEGR1, POMGNT2, TVP23B, WIPI2, MPP6, PTGDR2, PTPRT, RAB3B, PRICKLE1, KCNQ4, PTPRN, LANCL2, PTH2R, VEPH1, KIRREL3, PRKAR2B, CNEP1R1, GOLGA2, OBSCN, AATK, NECAB2, SLC17A6, SPRED1, TRPM2, SHISAL2B, SERAC1, YRDC, TVP23C, VAC14, TSPAN7, TSPAN13, TTC7B, TNS2, TBC1D3, ZDHHC13, SLC30A8, SLC25A25, STK10, GOPC, SCAMP5, PTPRN2, PHACTR2, SLCO2B1, SYT7, RAB29, NAPG, TEX261, MAPK10, PRRT3, SLC35G2, OPRD1, NAPB, SIGLEC9, TMEM39B, EMC4, DGCR2, CLTRN, FKBP2, GRK5, GNB5, CREB3L2, GLT8D2, FAM169A, FBXO2, CASR, AQP8, CD99, DIAPH1, CD82, DGKQ, GBP2, MAGI1, CXCR1, CDC42EP1, UGCG, CD81, ELMO2, CLU, FXYD2, ARRB1, AMPH, CADPS, CRHR1, CD1D, ARRDC1, ASIC5, AP1S3, EPB41L3, ADGRV1, GAD2, ALOX5, IBTK, ABCC5, SMPD4, SLC16A6, GFRA2, MAP2K1, KIAA2013, IGFLR1, FMO1, GAL3ST1, LDLRAD2, PI4KA, OR10R2, PRKCA, PSKH1,
   j. Tissue protected: Pancreatic exocrine cells AXL, TCTN2, CLEC10A, CD300C, CORO7, GABARAPL2, RASL10B, TBC1D3D, TMEM100, TM4SF18, UBE2J1, SELPLG, SYT13, PCSK5, PLSCR4, PI4KB, SVEP1, SPCS1, TNFSF12, STIMATE, NAAA, SLC27A2, TMEM97, SEC16B, POMGNT2, PLEKHA3, TVP23B, WHAMM, MED28, MPP6, PTPRT, PSCA, KCNH8, MRGPRF, SPATA9, SLCO4A1, STYK1, SCUBE1, MRGPRX2, PTH2R, NEURL1, PALM, PLPP7, VEPH1, PRKAR2B, CNEP1R1, SORD, GOLGA2, OBSCN, AATK, SPRED1, TMEM72, TRPM2, UGT3A1, VSIG10, SERAC1, YRDC, TVP23C, VAC14, TSPAN13, UNC13D, TTC7B, TBC1D3, TBC1D3E, ZDHHC13, ZC4H2, ARHGAP17, C2orf88, GOPC, SPHK1, SYCN, SCAMP5, SLC39A14, PHACTR2, RAB11FIP5, SLCO2B1, SYT7, RAB29, SLC4A4, NAPG, RYK, SEC11C, SLC19A1, PKHD1, NAPB, TRAF3IP3, TCTN3, TMEM39B, CA2, DTL, GGT1, DPEP1, CLSTN1, CUZD1, GLRB, SLC2A12, KIRREL2, DSP, ADCY7, CYSTM1, FPR2, FKBP2, GRK5, CHDH, CREB3L2, GLT8D2, EVI2A, FBXO2, GATM, FCGR2B, GCHFR, AADAC, AQP8, CD248, DIAPH1, FAM210A, GPAT2, CA12, CA4, FMO3, MALL, DGKQ, GBP2, LRRC37B, FES, MAGI1, LEPROTL1, MAN1C1, AP3M1, FADS3, ANKRD27, AQP1, BRI3, CDC42EP1, CDH9, ELOVL7, CD81, CLN3, ACE2, CNTNAP3, CFTR, FXYD2, ARRB1, CMKLR1, BSN, CADPS, CTSL, ARRDC1, CHST4, AKAP10, AP1S3, ADGRV1, PIK3AP1, HSD3B7, ALG14, CASQ1, AQP12A, AQP12B, IBTK, SMPD4, LRFN5, MYZAP, NCAM1, NDUFS1, GFRA2, MCUB, GNG12, MAP2K1, SLC2A6, KIAA2013, NIPA2, IGFLR1, FAM234B, GAL3ST1, RPS6KC1, OR10R2, PRKCA, PSKH1, LRRK2,
3. Cancer treated: renal cancer
   a. Tissue protected: Colon TRDN, TJAP1, UGT2A3, TMEM138, TECR, TMEM237, TMCC1, SLC7A6, MS4A8, MAP1LC3A, ATP8B2, CD36, MGAT4C, SLC46A1, ST3GAL6, SLC14A1, TMEM106B, UBE2J1, SNTG2, PCSK5, STX1A, RHOV, LPCAT2, SMIM24, SLC5A6, SUSD3, SLC6A6, PITPNM1, SEC13, SLC41A2, SOCS7, PEX1, PEMT, QRFPR, RPH3AL, NPDC1, NTRK1, RALGPS2, GOLGA8B, GRIK2, MPP7, RAC1, SERINC3, PCDHGA10, RHOF, HRAS, PLA2G4F, PILRA, RAC3, PKHD1L1, RGS16, MUC12, USP8, VPS26B, YIF1B, WHAMM, PTPRD, KLRG1, PARVB, PLCB3, PCDHA4, RHBDL2, SLC25A20, PTPRT, PSTPIP2, RAB3B, PLA2G2A, PSCA, KRAS, MRGPRF, TMEM45A, STXBP6, TPSG1, TRPM7, SLC18A1, NRAS, STYK1, TMEM134, SLC9A3, IFI27, ITLN1, TENM3, SYNGR4, STXBP3, TAOK2, MITD1, MUC15, PRSS8, PEX10, GNPNAT1, MRGPRX2, OSBPL7, CD46, LRRC55, PROM2, NUTF2, PALM, PLPP7, KCNQ1, SLC8A1, NAALADL2, SORD, NECTIN1, OBSCN, LDLRAD3, NALCN, SLC51B, SLC26A3, SLC43A3, SELENOT, SLC9A1, ST7, SNTA1, SCARF1, TMEM132A, UGT2B17, VSIG10, SYT11, TMEM236, SLC7A7, TUB, TEX264, TM4SF20, TMEM143, TMEM119, VPS35L, UGT2B28, ZP2, ZNRF1, QTRT1, TMEM69, TMEM253, TMEM41A, TSPAN8, PLAUR, ZFYVE9, UGT2B15, SLC35A3, SEZ6, GOPC, PCDH1, RNF125, TMTC4, QPCTL, SLC28A3, SLC45A4, SCAMP5, RAB27A, SYP, SLC12A6, SORT1, PARD3B, PIGZ, PTPRN2, SNX6, SMIM6, PHACTR2, MIA3, STK17B, SIGLEC14, SSTR1, TMEM204, PPM1L, SNX18, SLC23A1, SEC11C, SH3KBP1, PIK3R5, PHLPP2, RHOU, SLC4A5, SLC15A3, MSMO1, ARHGAP27, NAPB, RNF186, TRAF3IP3, TMEM154, TMIGD2, TYRO3, TAS2R38, TCTN3, TRPC6, UBL3, ZBED3, SLC30A10, CA2, PRKCZ, ACVR1B, DPEP1, EMC9, FAM83B, GSDMB, GPBAR1, FKRP, FMO5, EMC6, SLC2A12, SYK, KCTD3, FPR1, ARHGEF1, GOLPH3, ATP2C2, ABCG2, GPC5, CD177, CLCA1, GLP2R, CEACAM1, DCBLD1, EPHB3, CYP4F12, CYP4F3, ADGRF1, FAM126B, RASGRP2, CAPRIN2, EPHA4, DGKE, ENOX2, GCSAM, CALN1, CYP4F11, GBF1, DMTN, GOLGA8A, AQP8, ATP11B, ADGRE1, CEACAM5, WDPCP, CHODL, CNR1, ARFGEF3, CARMIL2, CA4, CHMP5, DEPDC5, KPNA2, FAM241B, GDAP1, GNG11, GPR82, IGSF11, IL17RC, LRRC37B, EVA1B, CPT P, HSD17B2, SLC7A8, MAGI1, FPGS, LPP, MAN1C1, FADS3, ABHD16A, CXCR1, EPHA10, FER1L6, ATP5F1C, BEST2, BRI3, CDC42EP5, MPP1, GAL3ST2, CDC42EP1, CYP4F2, KDELR3, FERMT1, CLN3, CRB3, ABCA10, CNTNAP3, CDH17, CD300LF, CDH5, DTX3L, CYB561, SLC25A31, CLDN11, KCNIP3, ATP1A2, ATP2A2, CADPS, AVEN, CERS6, CD300A, CXADR, GRAMD1A, CIB1, ORAI1, ADGRG6, CEACAM6, DCXR, ACKR2, ARL4C, CHST5, ARHGEF4, ABHD12, CNPPD1, EBP, BLNK, B3GNT7, ALG14, CHMP2B, CEACAM3, ALDH3A2, CYP2C9, DIP2A, IBTK, ABCC5, SMPD4, SLC9A3R2, NISCH, LRFN5, SLC16A6, MYZAP, LHFPL2, KCNN3, GIPC1, NDUFS1, GPR153, MS4A12, FUT3, GFRA2, GLUL, HSP90AA1, IL17RE, KIAA1109, MCUB, MARCKSL1, MTCH2, EFHD2, FUT6, GPER1, SLC2A3, HCN3, MBTPS1, JAML, NIPAL1, IGFLR1, IL17RA, ITGB4, LY9, LYN, GPA33, GPC1, RPS6KC1, LZTR1, ALOX12, MUC13, NFXL1, MYOC, NEU4, OR10R2, MRC1, KLRC3, MUC4, NAPEPLD, IL4R,
   b. Tissue protected: Lung TJAP1, SLC30A4, SLC7A6, EFNB3, ST3GAL6, SLC14A1, TRIM13, UBE2J1, LPCAT2, SUSD3, SERINC3, TRPV6, SLC18A2, WHAMM, SLC25A20, PTPRT, REEP2, PTPRG, STXBP6, MGAT4B, STX5, TENM3, MRGPRX2, OSBPL7, CD46, NUTF2, PALM, KCNQ1, EHD2, NECTIN1, OBSCN, PPL, SNTA1, TMEM80, TMEM119, ZNRF1, QTRT1, TBC1D3, SEZ6, GOPC, PCDH1, RNF125, TMTC4, QPCTL, RHOG, PSD4, PHACTR2, TM4SF1, STEAP1, SNX18, S100A9, PIK3R5, PHLPP2, RHOU, SIGLEC9, UBL3, CA2, ACVR1B, EHD1, GJB4, GPBAR1, FPR1, CD247, ARHGEF1, CCR7, ATP2C2, SLC44A2, GPC5, DCBLD1, ECEL1, EPHB3, CLDN18, EPHA4, DGKE, EPHX1, CALN1, HCLS1, DMTN, CEACAM5, CCDC88A, GPR82, IL17RC, AIFM2, EVA1B, CLIC2, CXCR1, DGKZ, CDC42EP1, CYB561D1, DTX3L, CYB561, C ACNA2D2, ACE, CEACAM6, BDKRB2, CXCR3, IBTK, LAMP3, MARCKSL1, EFHD2, EHD3, GPER1, GALNT13, NSMF, IL4R,
   c. Tissue protected: Skin TMEM243, TRDN, TJAP1, SUN3, TMEM138, TMEM237, TMCC1, CLEC10A, MGAT4C, ST3GAL6, PERP, STRA6, RXFP1, SOX10, SNTG2, RHOV, SLC5A6, SLC6A1, SLC6A6, PITPNM1, SEC13, SLC41A2, TMEM97, NPDC1, RALGPS2, GRIK2, MPP7, RAC1, NTM, PTGS2, SLC26A9, RAC3, PKHD1L1, RGS16, TMEM40, VPS26B, MTMR2, RHBDL2, REEP2, TMEM45A, MGAT4B, STX5, STYK1, TMEM134, PCDH19, SLC10A6, SLC9A3, IFI27, TENM3, STXBP3, TAOK2, MITD1, MUC15, NEU3, OLFM2, KCNAB1, LRRC55, PROM2, NUTF2, PALM, PLPP7, LAX1, LYPD3, KRT1, PLA2G4E, SLC8A1, NAALADL2, SORD, DMPK, NECTIN1, OBSCN, PPL, SLC43A3, TRPM2, SELENOT, SLC9A1, SNTA1, TMEM79, SLC7A7, SRC, TUB, YRDC, ZP2, ZNRF1, TMEM69, THBD, TACSTD2, UGT2B15, SLC35A3, GOPC, RNF125, TMTC4, QPCTL, SLC28A3, SLC45A4, S1PR5, PARD3B, SMIM6, PHACTR2, MIA3, NGFR, SSTR1, TMEM204, SNX18, SNORC, SH3KBP1, LY6D, S100A9, RHOU, SELENOK, MSMO1, NAPB, RIPK4, SIGLEC9, TRAF3IP3, TYRO3, TAS2R38, TRPC6, COL17A1, EMC9, FAM83B, CYBA, GJB4, SYK, FA2H, FPR1, ARHGEF1, CYP2W1, ATP2C2, DSP, GPC5, CD177, GLP2R, ANO7, DCBLD1, FYN, FRS3, EPHB3, CYP4F12, CYP4F3, FAM126B, EPHA4, GPR84, ENOX2, CALN1, DMTN, FAT2, CD99, IL1RAP, CD248, WDPCP, CAPNS2, ARFGEF3, CARMIL2, KPNA2, MALL, GDAP1, DEF6, DSC3, GSDMA, FGFBP1, LRRC37B, EVA1B, SLC7A8, LPP, FADS3, EPHA10, B3GNT4, DSG1, BRI3, CDC42EP1, KDELR3, FERMT1, ABCA10, CCR2, CDH5, DTX3L , CYB561, SLC25A31, MS4A1, AVEN, CAV3, CXADR, CD1D, ASPRV1, CIB1, ORAI1, ACSBG1, DSG3, DCXR, ACKR2, CLEC2A, CNPPD1, ATP12A, BLNK, ALDH3A2, KCNJ11, IBTK, ABCC5, NISCH, MYZAP, GIPC1, GPR153, IL17RE, KIAA1109, MCUB, MARCKSL1, FGFR3, GPER1, NIPAL1, TACR1, ITGB4, GPC1, RPS6KC1, ALOX12, OR10R2, NCS1, IL4R, TMEM243, TCTN2, SLC30A4, TECR, MS4A8, TRIM13, UBE2J1, ULBP3, LPCAT2, SUSD3, TYR, PEMT, NTRK1, GOLGA8B, HRAS, PLA2G4F, RHBG, USP8, YIF1B, PLN, PTPRT, KRAS, RASA4, NRAS, TECTA, GNPNAT1, OSBPL7, CD46, ICAM3, KCNQ1, ST7, SMIM18, TMEM80, TMEM143, TMEM119, QTRT1, TBC1D3, DCT, PRNP, SNTB1, SH2D3C, PCDH1, RHOG, TM4SF1, STK17B, PMEL, SEMA5B, PHLPP2, ARHGAP27, TMIGD2, TCTN3, UPK3A, DTL, CNTN3, EHD1, GSDMC, KCTD3, IGSF3, ADCY7, EVI2A, TM7SF2, GBF1, CYB561A3, FCGR2B, GOLGA8A, FAM210A, ADGRE1, CEACAM5, GPAT2, CD82, CNR1, DNER, FGD2, FAM241B, IGSF11, IL17RC, CPTP, CXCR1, ATP5F1C, DGKZ, FNBP1, CYB561D1, ATP2A2, CERS6, CDH13, ATG2A, BDKRB2, B3GNT7, ALG14, ADGRA1, CHMP2B, LHFPL2, LHFPL6, MUC21, NDUFS1, GFRA2, EFHD2, EHD3, JAML, IL17RA, KIT, LZTR1, KIAA0040, ,
   d. Tissue protected: Liver TRDN, TJAP1, TMEM138, TECR, TMCC1, MAP1LC3A, CLEC10A, CREB3L3, ST3GAL6, SLC14A1, STRA6, TRIM13, UBE2J1, PCSK5, SLC6A4, LPCAT2, SYNE3, SUSD3, SLC25A40, SLCO1B3, SLC41A2, SLCO1B1, PEMT, SLC27A5, NPDC1, NTRK1, SERINC3, SLC18A2, PTPRD, KSR2, MTMR2, PTGDR2, SLC25A20, PTPRT, RDH16, LIN7A, TMEM45A, MGAT4B, RASA4, STX5, PCDH19, TENM3, SYNGR4, TAOK2, MITD1, PRRT2, GNPNAT1, MRGPRX2, CD46, OLFM2, LRRC55, KCNQ1, SLC8A1, SORD, NECTIN1, OBSCN, LDLRAD3, RFTN1, SELENOT, TMEM132A, TFR2, TUB, VPS35L, ZNRF1, TNS2, UGT2B15, SLC25A25, ST7L, GOPC, PCDH1, SLC45A4, SORT1, PARD3B, MIA3, SLCO2B1, TMEM204, PPM1L, TEX261, MSMO1, NAPB, SIGLEC9, TYRO3, TM4SF4, SLC30A10, CA2, ACVR1B, GSDMB, FKRP, FMO5, GLRB, CYP2W1, CYP2D6, GJA5, CD177, CEACAM1, CACNA1A, FYN, GAS2, EPHB3, CYP4F12, CYP4F3, RASGRP2, CAPRIN2, CYP2A7, EPHA4, HEPACAM, EPHX1, CYP4F11, FOLH1, CYP1A2, DMTN, CYP3A4, FAM210A, CEACAM5, CYP2A6, CARMIL2, CYP4V2, DEPDC5, KPNA2, MALL, GPRC5B, IL17RC, NINJ1, CYP2C8, DHCR24, LRRC37B, EVA1B, GPAM, HSD17B2, MAL2, MAN1C1, CYP2C19, FADS3, AQP9, ATP5F1C, BRI3, CYP4F2, FERMT1, CRB3, ASGR1, CYB561D1, CDH5, DTX3L, ABCB11, ATP1A2, CYP2A13, AVEN, CYP2E1, CXADR, GRAMD1A, DCXR, EPB41L5, HSD11B1, BACE2, CD320, EBP, ASGR2, ATG2A, CYP3A43, CHMP2B, ALDH3A2, KCNJ11, CYP2C9, IBTK, SMPD4, SLC9A3R2, NISCH, SLC16A6, MYZAP, LHFPL2, MFAP3L, GIPC1, LIMS2, NDUFS1, GLUL, IL17RE, MTCH2, GPR15, HCN3, MBTPS1, NIPAL1, IGFLR1, IL17RA, ITGB4, RPS6KC1, LZTR1, ALOX12, OR10R2, ABCC2, NAPEPLD, NPC1L1,
   e. Tissue protected: Kidney (glomeruli) ZDHHC16, TMEM138, TECR, TMCC1, ZAP70, MGAT4C, ST3GAL6, SLC14A1, UBE2J1, KDR, SYNE3, SUSD3, SLC45A1, PEMT, RALGPS2, RAC1, PODXL, PLA2G4F, PILRA, PDGFRB, RAC3, PKHD1L1, TRPV6, USP8, YIF1B, WHAMM, KSR2, PARVB, PTPRT, PTPRG, MRGPRF, SLC34A1, TPSG1, STX5, PECAM1, SCUBE1, TECTA, TENM3, PLA2R1, CD46, PTH2R, MCAM, NEU3, KCNAB1, LRRC55, PROM2, NUTF2, PALM, MAST1, KCNQ1, MAP6, NOS3, EHD2, MPP5, NECTIN1, LDLRAD3, NPHS2, SLC43A3, SLC9A1, ST7, SNTA1, SCARF1, SMIM18, TUB, TMEM119, VPS35L, ZNRF1, TSPAN7, TTC7B, TNS2, ZFYVE9, STK10, GOPC, PCDH1, RNF125, SNCA, SLC45A4, PSD4, SORT1, PARD3B, SNX6, PHACTR2, STK17B, THSD7A, SNX18, PITPNM3, PTPRO, RHOU, NAPB, TYRO3, ZBED3, PRKCZ, FAM83B, EHD1, ATP2C2, GPC5, CEACAM1, DCBLD1, FYN, GAS2, FAM107A, ADGRF1, CD34, FAM126B, RASGRP2, EVI2A, CALN1, DMTN, ENG, CD99, CD248, DOCK5, FAM210A, ADGRE1, CR1, FGD2, ARFGEF3, FAM241B, GPR82, GPRC5B, EVA1B, MAS1, LPP, ADGRL4, CLIC2, FADS3, FNBP1, C DC42EP1, CBL, FERMT1, CLN3, CRB3, ACKR1, DTX3L, CHRM1, CERS6, CXADR, CDH13, EPB41L5, BBS7, SLC9A3R2, GIPC1, LIMS2, GPR153, IL17RE, I TGA8, MARCKSL1, EFHD2, EHD3, GPER1, SLC2A3, MBTPS1, MLIP, JAML, GPC1, LZTR1, NFXL1, KIAA0040, NEU4, NAPEPLD, PDGFRA,
   f. Tissue protected: Kidney (tubules) TMEM231, TMEM243, TRDN, TJAP1, SUN3, TRPM3, TMEM138, TECR, TMEM237, TMCC1, TBC1D3G, TREH, MS4A8, ATP8B2, MGAT4C, ST3GAL6, THY1, SLC14A1, STRA6, UBE2J1, SELPLG, PCSK5, SLC6A4, SMIM24, SYNE3, SUSD3, SLC6A6, STIMATE, SLC12A1, TLN2, PCDHB2, PITPNM1, SLC25A40, SLC41A2, PEX1, PEMT, QRFPR, RPH3AL, SLC34A3, NTRK1, RALGPS2, GOLGA8B, GRIK2, MPP7, SERINC3, SLC28A2, RHOF, RDH11, HRAS, PTH1R, PLA2G4F, PTGER3, PKHD1L1, RHBG, RGS16, USP8, VPS26B, YIF1B, WHAMM, PTPRD, PARVB, RHBDD2, PLCB3, PCDHA4, PDE2A, RHBDL2, SLC25A20, PTPRT, PSCA, KRAS, LIN7A, MRGPRF, TMEM45A, STXBP6, MGAT4B, SLC34A1, TRPM7, RASA4, STX5, PRICKLE1, NRAS, STYK1, TMEM134, PCDH19, SLC9A3, IFI27, TENM3, STXBP3, MITD1, MUC15, PEX10, PRRT2, GNPNAT1, OSBPL7, CD46, NEU3, OLFM2, KCNAB1, LRRC55, PROM2, NUTF2, PANX2, PALM, PLPP7, KCNQ1, ITPR1, SLC8A1, NAALADL2, SORD, EHD2, MPP5, NECTIN1, OBSCN, LDLRAD3, NALCN, PPL, NKAIN4, SLC51B, RFTN1, SLC43A3, TRPM2, SELENOT, SLC9A1, UGT3A1, ST7, SNTA1, TMEM132A, VSIG10, ATP6V0A4, SLC7A7, SRC, TUB, TMEM80, TMEM119, VPS35L, ZP2, ZNRF1, QTRT1, TMEM252, TMEM174, TTC7B, TBC1D3, TMEM41A, TSPAN8, PLAUR, ZFYVE9, FASLG, SEMA6B, SNTB1, SIRT2, SLC22A2, SLC52A3, SH2D3C, GOPC, PCDH1, RNF125, QPCTL, RHOG, SLC28A3, SLC45A4, SCAMP5, SLC22A8, SORT1, PARD3B, PIGZ, SNX6, SMIM6, PHACTR2, SLC6A18, SLC13A2, TMEM213, MIA3, SLC5A1, STK17B, SIGLEC14, SSTR1, TMEM204, TMEM184A, SLC4A4, SNX18, SNORC, SLC23A1, SEC11C, TEX261, PIK3R5, PKHD1, RHOU, SLC35G2, SLC4A5, MSMO1, OPRD1, NAPB, RIPK4, TMEM242, TMIGD2, TYRO3, TAS2R38, TSPAN16, UPK3A, UBL3, ZBED3, CA2, ENPP6, EMC4, PRKCZ, ACVR1B, IYD, DPEP1, EMC9, FAM83B, EHD1, ERMP1, GSDMB, DLK1, GPBAR1, FKRP, FMO5, EMC6, GLRB, GSDMC, SYK, ARHGEF1, CPNE6, ATP2C2, SLC44A2, GJA5, GPC5, ADCY7, CD177, DCBLD1, GAS2, FAM107A, EPHB3, FNDC5, CYP4F12, CYP4F3, DOCK2, CLCNKB, FAM126B, CLCNKA, EPHA4, ENOX2, EVI2A, FAM169A, TM7SF2, CASR, CYP4F11, FOLH1, GBF1, DMTN, GOLGA8A, ATP11B, IL1RAP, CD248, FAM210A, ADGRE1, WDPCP, FRRS1L, CARMIL2, CA4, CHMP5, CYP4V2, DEPDC5, KPNA2, FAM241B, GDAP1, GNG11, GLIPR1L2, LPXN, GPR82, KIAA0319, GPRC5B, IGSF11, DHCR24, LRRC37B, LRFN4, CPTP, SLC7A8, MAL2, MAGI1, FPGS, LPP, MAN1C1, FADS3, ABHD16A, BSND, CXCR1, EPHA10, AQP6, ATP5F1C, DSG1, BRI3, CDC42EP5, MPP1, CYP4F2, FERMT1, CDC42SE1, CRB3, CCR2, CDH5, CLDN10, DUSP15, DTX3L, SLC25A31, ACE, CATIP, ATP2A2, AVEN, CERS6, CAV3, CPT1B, COX7B, CXADR, GRAMD1A, CIB1, DCXR, ACKR2, ARL4C, ARHGEF4, EPB41L5, EBP, ATP12A, BLNK, C5AR1, CYP3A43, CHMP2B, ALDH3A2, DIP2A, IBTK, ABCC5, SMPD4, LRFN2, NISCH, SLC16A6, MYZAP, LHFPL2, KCNJ10, KCNJ12, MFAP3L, GIPC1, NDUFS1, FUT3, GFRA2, IL17RE, KIAA1109, MCUB, MARCKSL1, MTCH2, EHD3, FUT6, GPER1, HCN3, MBTPS1, MS4A4A, MLIP, JAML, NIPAL1, IGFLR1, IL17RA, FMO1, GPC1, RPS6KC1, LZTR1, ALOX12, NFXL1, KIAA0040, MYOC, NXPE2, IL23R, OR10R2, MRC1, NSMF, ABCC2, NAPEPLD, KCTD8, IL4R, NPC1L1,
   g. Tissue protected: Heart (cardiomyocytes) TJAP1, TCTN2, SLC30A4, TMEM138, TECR, TMEM237, TMCC1, SLC7A6, EFNB3, ATP8B2, CD36, MGAT4C, SLC46A1, RASL10B, ST3GAL6, SLC14A1, STRA6, TMEM106B, PCSK5, LPCAT2, SUSD3, SLC6A6, TLN2, PITPNM1, SEC13, SLC41A2, PEX1, PEMT, NPDC1, NTRK1, RALGPS2, GRIK2, RAC1, SERINC3, PLA2G4F, RAC3, PKHD1L1, USP8, VPS26B, YIF1B, WHAMM, PTPRD, PARVB, POPDC2, P2RY6, SLC25A20, PLCH2, PLN, MRGPRF, SHISA4, MGAT4B, RASA4, SGCG, SRL, PCDH19, SLC9A3, TECTA, TENM3, MITD1, PRSS8, PRRT2, OSBPL7, CD46, MCAM, OLFM2, LRRC55, ART3, PLPP7, KLHL41, KCNQ1, ITPR1, SLC8A1, DMPK, EHD2, NECTIN1, OBSCN, SLC43A3, SIPA1, RYR2, SELENOT, SLC9A1, ST7, SNTA1, TMEM132A, TMEM236, SLC7A7, SRC, YRDC, TMEM143, TMEM119, TREML1, VPS35L, ZP2, ZNRF1, QTRT1, TTC7B, TBC1D3, TMEM41A, SEMA6B, SLC25A25, SNTB1, SIRT2, SH2D3C, GOPC, PCDH1, QPCTL, SLC45A4, SCAMP5, RAB27A, RAB11FIP4, SORT1, PIGZ, RYR1, SLAMF8, SNX6, SMIM6, PHACTR2, TM4SF1, MIA3, SLCO2B1, STK17B, TMEM204, PPM1L, SEMA5B, TEX261, RHOU, SLC35G2, SLC15A3, MSMO1, NAPB, TYRO3, TAS2R38, TCTN3, TRPC6, UPK3A, UBL3, ZBED3, CNTN3, EMC9, EHD1, GSDMB, FKRP, EMC6, MMP27, KCTD3, FPR1, CYP2W1, GOLPH3, ATP2C2, DSP, ABCG2, GPC5, ADCY7, CDH15, ADRA1A, DCBLD1, FRS3, EPHB3, FNDC5, CYP4F12, CYP4F3, ADGRF1, FAM126B, RASGRP2, CAVIN4, CAPRIN2, SLC2A4, EPHA4, GPR84, DGKE, TM7SF2, HHATL, FAM168B, CALN1, GBF1, CYB561A3, FCGR2B, CHRNA1, IL1RAP, CD248, WDPCP, GPAT2, FGD2, ARFGEF3, CARMIL2, CHMP5, CYP4V2, DEPDC5, KPNA2, MALL, FAM241B, GPR82, CAMK2B, GPRC5B, IGSF11, IL17RC, LRRC37B, EVA1B, MBOAT7, CPTP, LPP, MAN1C1, ADGRL4, CLIC2, FADS3, ABHD16A, CXCR1, EPHA10, FCGRT, ATP5F1C, B3GNT4, DGKZ, DMD, FERMT1, CDC42SE1, CLN3, ATP1A3, CNTNAP3, CCR2, DTX3L, CSF3R, CYB561, SLC25A31, ATP1A2, ATP2A2, CADPS, AVEN, CAV3, CD300A, CPT1B, COX7B, CXADR, CDH13, CD1D, CIB1, ORAI1, AKAP6, ADGRG6, DCXR, ARL4C, ARHGEF4, BACE2, CNPPD1, BLNK, ALDH3A2, IBTK, JPH1, ABCC5, SMPD4, NISCH, SLC16A6, MYZAP, LHFPL2, MFAP3L, GIPC1, GPR182, LIMS2, NDUFS1, FUT3, IL17RE, KIAA1109, MCUB, MTCH2, EHD3, FUT6, GPER1, GPR15, HCN3, MBTPS1, MLIP, IGFLR1, ITGB4, IL18RAP, RPS6KC1, LZTR1, ALOX12, NFXL1, OR10R2, MCEMP1, NAPEPLD, DES, IL4R,
   h. Tissue protected: Thyroid TJAP1, TCTN2, SLC30A4, TMEM138, TECR, TMEM237, TMCC1, ATP8B2, SLC46A1, ST3GAL6, TMEM100, STRA6, TMEM106B, TRIM13, UBE2J1, VPS33B, PCSK5, TMEM251, LPCAT2, SLC6A6, TLN2, PCDHB2, SEC13, PEX1, PEMT, QRFPR, NPDC1, NTRK1, GRIK2, MPP7, SERINC3, HRAS, TPO, PKHD1L1, RGS16, TRPV6, TMEM40, VPS26B, YIF1B, WHAMM, RHBDD2, RHBDL2, SLC25A20, KRAS, LIN7A, STXBP6, PRICKLE1, NRAS, PCDH19, IFI27, TENM3, STXBP3, TAOK2, MITD1, MUC15, NMUR2, OSBPL7, CD46, NEU3, OLFM2, KCNAB1, PSD3, LRRC55, PROM2, NUTF2, PALM, PLPP7, MAST1, KCNQ1, SLC8A1, SORD, NECTIN1, OBSCN, LDLRAD3, NALCN, PPL, PORCN, SLC43A3, TRPM2, SELENOT, SLC9A1, ST7, SLC7A7, TUB, YRDC, TMEM80, TEX264, TMEM119, VPS35L, ZNRF1, QTRT1, TTC7B, TBC1D3, TMEM41A, ZFYVE9, FASLG, SLC25A25, SLC52A3, GOPC, PCDH1, RNF125, TMTC4, QPCTL, SLC45A4, SLC12A6, SORT1, PARD3B, SMIM6, PHACTR2, MIA3, SLCO2B1, STK17B, TMEM204, PPM1L, SNX18, SH3KBP1, TEX261, PIK3R5, RHOU, MSMO1, SYN3, SIGLEC9, TMIGD2, TYRO3, TCTN3, UBL3, ZBED3, C19orf18, PRKCZ, ACVR1B, IYD, CNTN3, DUOX1, EMC9, FAM83B, EHD1, SYK, KCTD3, CYP2W1, GOLPH3, ABCG2, GPC5, DCBLD1, FYN, EPHB3, CLCNKB, FAM126B, RASGRP2, CLCNKA, EPHA4, DGKE, GCSAM, FAM168B, CALN1, GBF1, DMTN, FCGR2B, CD99, IL1RAP, FAM210A, ADGRE1, WDPCP, GPAT2, CNR1, FGD2, ARFGEF3, CARMIL2, CHMP5, IPCEF1, DEPDC5, KPNA2, MALL, FAM241B, GDAP1, LPXN, CAMK2B, IGSF11, IL17RC, EVA1B, MAGI1, ADGRL4, CLIC2, FZD5, CXCR1, EPHA10, FER1L6, ATP5F1C, FNBP1, CDC42EP1, FERMT1, CRB3, ABCA10, DTX3L, CYB561, ATP2A2, CRHR1, AVEN, CERS6, CD300A, CXADR, CIB1, ORAI1, DCXR, ACKR2, ARL4C, ARHGEF4, CD320, BLNK, C5AR1, CDH10, CHMP2B, CEACAM3, ALDH3A2, AP3B2, IBTK, SMPD4, NISCH, MTNR1B, MYZAP, LHFPL2, GIPC1, NDUFS1, FUT3, GFRA2, GLUL, IL17RE, KIAA1109, MARCKSL1, FUT6, GPER1, GPR15, HCN3, MBTPS1, JAML, IGFLR1, IL17RA, ITGB4, LYN, LZTR1, OR10R2, NAPEPLD, NCS1,
   i. Tissue protected: Pancreatic endocrine cells (Islets) TJAP1, SLC30A4, STXBP1, MAP1LC3A, UBE2J1, STX1A, RAB3A, PITPNM1, SEC13, RTN1, PEMT, QRFPR, RPH3AL, NPDC1, NTRK1, SCG3, POMGNT2, PKHD1L1, RGS16, YIF1B, PTGDR2, RHBDL2, SLC25A20, PTPRT, RAB3B, STX5, PRICKLE1, STXBP3, TAOK2, MITD1, PTPRN, CD46, PTH2R, OLFM2, LRRC55, NUTF2, SLC8A1, NECTIN1, OBSCN, NECAB2, SLC17A6, TRPM2, SLC9A1, SMIM18, SV2A, TUB, YRDC, TM4SF20, ZNRF1, TMEM69, TSPAN7, TTC7B, TNS2, TBC1D3, PLAUR, ZFYVE9, SLC30A8, PRNP, FASLG, SLC35A3, SLC25A25, STK10, GOPC, PCDH1, RNF125, QPCTL, SCAMP5, RAB27A, SYP, SLC12A6, SORT1, PTPRN2, SMIM6, PHACTR2, MIA3, SLCO2B1, SIGLEC14, SSTR1, TMEM204, PPM1L, SNORC, TEX261, MAPK10, RHOU, SLC35G2, SLC4A5, SELENOK, MSMO1, OPRD1, NAPB, SIGLEC9, EMC4, PRKCZ, ERMP1, DLK1, FMO5, DGCR2, KCTD3, CYP2W1, GOLPH3, EPHB3, GNAO1, CAPRIN2, FAM169A, FBXO2, GCSAM, CASR, AQP8, CD99, WDPCP, CD82, ARFGEF3, CARMIL2, CHMP5, CYP4V2, KPNA2, FAM241B, GDAP1, IGSF11, IL17RC, SLC7A8, MAGI1, CXCR1, EPHA10, DGKZ, CDC42EP1, FERMT1, ABCA10, CDH5, CYB561, AMPH, ATP1A2, ATP2A2, CADPS, CRHR1, AVEN, CXADR, CD1D, ARL4C, ARHGEF4, EPB41L5, BBS7, GAD2, ALDH3A2, KCNJ11, IBTK, ABCC5, SMPD4, NISCH, SLC16A6, GIPC1, GPR153, FUT3, GFRA2, GLUL, IL17RE, MTCH2, FUT6, GPER1, HCN3, MBTPS1, IGFLR1, FMO1, OR10R2, NAPEPLD,
   j. Tissue protected: Pancreatic exocrine cells TJAP1, TCTN2, TMEM138, TMEM237, TMCC1, ATP8B2, CLEC10A, SLC46A1, RASL10B, ST3GAL6, TBC1D3D, TMEM100, STRA6, UBE2J1, SELPLG, PCSK5, SLC6A6, STIMATE, SEC13, SLC41A2, PEX1, PEMT, RPH3AL, TMEM97, NTRK1, GRIK2, SEC16B, HRAS, POMGNT2, PKHD1L1, RGS16, TMEM40, VPS26B, YIF1B, WHAMM, PCDHA4, RHBDL2, SLC25A20, PTPRT, PSCA, KRAS, MRGPRF, STXBP6, STX5, NRAS, STYK1, SCUBE1, IFI27, TENM3, STXBP3, MITD1, MUC15, GNPNAT1, MRGPRX2, CD46, PTH2R, NEURL1, OLFM2, KCNAB1, LRRC55, PROM2, NUTF2, PALM, PLPP7, KCNQ1, SLC8A1, SORD, NECTIN1, OBSCN, LDLRAD3, NALCN, PPL, RFTN1, SLC43A3, TRPM2, SELENOT, SLC9A1, UGT3A1, ST7, SNTA1, VSIG10, YRDC, TMEM80, TMEM119, ZNRF1, UNC13D, TTC7B, TBC1D3, TRAT1, TMEM41A, TSPAN8, PLAUR, TBC1D3E, ZFYVE9, TACSTD2, FASLG, SNTB1, GOPC, PCDH1, RNF125, TMTC4, SYCN, QPCTL, SLC28A3, SLC45A4, SCAMP5, RAB27A, SLC12A6, PARD3B, SMIM6, PHACTR2, TM4SF1, MIA3, SLCO2B1, SSTR1, TMEM204, SLC4A4, SEC11C, PKHD1, RHOU, SLC4A5, SELENOK, ARHGAP27, NAPB, TRAF3IP3, TAS2R38, TCTN3, TM4SF4, CA2, DTL, PRKCZ, ACVR1B, DPEP1, EMC9, EMC6, GLRB, GP2, SLC2A12, SYK, KCTD3, FA2H, GOLPH3, DSP, ADCY7, CYSTM1, EPHB3, ADGRF1, RASGRP2, DGKE, EVI2A, FBXO2, GCSAM, EPHX1, GBF1, FCGR2B, AQP8, ATP11B, CD248, FAM210A, WDPCP, GPAT2, FGD2, ARFGEF3, CARMIL2, CA4, CHMP5, CYP4V2, KPNA2, MALL, GPR82, GPRC5B, IGSF11, IL17RC, LRRC37B, EVA1B, MAGI1, FPGS, MAN1C1, FADS3, FZD5, EPHA10, ATP5F1C, BRI3, CDC42EP1, KDELR3, FERMT1, CDC42SE1, CLN3, CRB3, ABCA10, CNTNAP3, CDH5, CFTR, CLDN10, CYB561, SLC25A31, CADPS, AVEN, AQP5, CD300A, CXADR, CIB1, CHST4, ARL4C, B3GNT7, ALG14, BBS7, CHMP2B, AQP12A, AQP12B, IBTK, SMPD4, NISCH, LRFN5, MYZAP, GIPC1, NDUFS1, FUT3, GFRA2, IL17RE, KIAA1109, MCUB, MARCKSL1, MTCH2, EFHD2, FUT6, GPER1, MBTPS1, IGFLR1, IL17RA, LYN, RPS6KC1, NFXL1, MYOC, OR10R2, NAPEPLD,
4. Cancer treated: urothelial cancer
   a. Tissue protected: Colon TMEM158, UGT2A3, TMEM60, MS4A8, MAP1LC3A, CACNB2, CD36, ILK, MGAT4C, SCGN, TMEM106B, UBE2J1, SLC47A1, SYT13, PCSK5, STX1A, RAB17, ENPP1, SMIM24, SLC27A2, SH3BP4, SLC19A3, MGAM, PEX11G, BVES, P2RX6, PILRA, MUC12, VPS26B, KLRG1, PARVB, DLC1, RNF144B, RAB3B, PSCA, MRGPRF, TPSG1, TRPM7, SLC18A1, SPATA9, SLCO4A1, STYK1, SLC9A3, IFI27, ITLN1, PTGDR, SYNGR4, SYBU, STXBP3, MITD1, PEX10, OSBPL7, MYORG, MCOLN1, LRRN2, OBSCN, LDLRAD3, AATK, UGT2B4, TMEM72, TMEM132A, UGT2B17, VSIG10, RNF112, SYT11, TMEM236, SLC7A7, UST, UGT2B28, QTRT1, SLC16A14, TMEM253, UNC13B, PLAUR, PRRG2, UGT2B15, SLC35A3, SEZ6, SCAMP5, RAB27A, RAB43, PTPRN2, RUFY3, SNX6, SEC11C, PIK3R5, SCN7A, ARHGAP21, ARHGAP27, NAPB, RNF186, TMEM219, TRAF3IP3, VASN, UBL3, TMEM39B, CRK, CSK, CA2, PRKCZ, GGT1, DPEP1, GSDMB, IL9R, AIF1L, ADCY10, BMPR1A, CLCA1, GLP2R, AP4M1, FPR2, FKBP2, CHDH, ADGRF1, FAM126B, HTR1A, AKR1A1, ENOX2, CALN1, CHODL, CA4, CYP4X1, DEPDC5, GNG11, KCNMA1, LRRC37B, EVA1B, CPTP, HSD17B2, MAGI1, CXCR1, ANKRD13B, AQP1, BEST2, ABCG8, CDC42EP5, BCL2, MPP1, GAL3ST2, HTR4, B3GNT8, ELOVL7, AP1S2, CDHR2, CLN3, CRB3, ANPEP, CNTNAP3, CDH17, C3AR1, CLDN11, KCNIP3, ATP1A2, CADPS, GRAMD1A, ADGRG6, ASIC5, CEACAM6, ACKR2, ANXA13, BBS1, ARHGEF4, ASPHD1, ABHD12, C12orf66, BLNK, PAM, ALG14, CEACAM3, ALDH3A2, B3GAT1, CYP2C9, DIP2A, IBTK, LRFN5, MYZAP, NDUFS1, ALOX15B, MS4A12, MCUB, NIPAL4, IGFLR1, LY9, GAL3ST1, GPA33, RPS6KC1, LZTR1, MUC13, HPS6, NEU4, OR10R2, MRC1, KLRC3,
   b. Tissue protected: Lung VSIG4, SLC30A4, EFNB3, CACNB2, CD300C, ILK, TRIM13, UBE2J1, SLC22A15, SLC25A17, RAB17, SLC19A3, BVES, SLC18A2, IL10RB, REEP2, RNF144B, PTPRG, SGIP1, SYBU, MXRA7, OSBPL7, KIRREL3, EHD2, OBSCN, CD84, QTRT1, SLC16A14, TBC1D3, PRRG2, SEZ6, RHOG, PSD4, RAB43, RUFY3, STEAP1, PIK3R5, SCN7A, SIGLEC9, UBL3, CA2, EHD1, CD247, CCR7, SLC44A2, AOC3, ECEL1, FKBP2, CLDN18, CALN1, KCNMA1, AIFM2, EVA1B, FES, CLIC2, CXCR1, ANKRD13B, AQP1, ABCG8, ANK2, AQP4, C3AR1, CACNA2D2, ACE, CEACAM6, ASPHD1, CADM1, CXCR3, IBTK, LAMP3, ALOX15B, EHD3, HPS6, NSMF, LRRK2,
   c. Tissue protected: Skin SUN3, CACNB2, CLEC10A, CD300C, MGAT4C, PERP, STRA6, SLC47A1, SOX10, SLC25A17, RAB17, ENPP1, SLC6A1, SH3BP4, VPS26B, IL10RB, REEP2, RNF144B, GPIHBP1, TYROBP, STYK1, SLC9A3, IFI27, STXBP3, MITD1, MXRA7, LANCL2, F2R, MCOLN1, LRRN2, OBSCN, AATK, TRPM2, RMC1, SLC7A7, YRDC, SLC16A14, PRRG2, UGT2B15, SLC35A3, ARHGAP21, NAPB, SIGLEC9, TRAF3IP3, TMEM39B, CRK, CYBA, FFAR2, FA2H, CYP2W1, ADCY10, BMPR1A, CDH6, GLP2R, CACNA2D1, FYN, FPR2, FKBP2, FAM126B, ENOX2, ATRAID, CALN1, IL1RAP, MALL, DEF6, LRRC37B, EVA1B, B3GNT4, DSG1, ABCG8, ELOVL7, CD81, ANK2, MS4A1, CD1D, ASPRV1, ASIC5, DSG3, ACKR2, ASPHD1, BLNK, PAM, ALDH3A2, IBTK, ABCC1, MYZAP, MCUB, NIPAL4, RPS6KC1, OR10R2, NCS1, TCTN2, SLC30A4, MS4A8, APLNR, TRIM13, UBE2J1, SYT13, TYR, DLC1, PLN, SLCO4A1, KCNQ4, SGIP1, OSBPL7, MYORG, SLC25A15, UST, QTRT1, TBC1D3, DCT, UNC13B, PRNP, RHOG, RUFY3, PMEL, SEMA5B, ARHGAP27, TMEM219, EHD1, AIF1L, TM7SF2, C YB561A3, GPAT2, DNER, FGD2, CYP4X1, CPTP, CXCR1, BCL2, B3GNT8, CDH13, BBS1, CPNE2, AP4E1, ATG2A, ALG14, MUC21, NDUFS1, EHD3, LZTR1, ,
   d. Tissue protected: Liver VNN1, SLC13A5, MAP1LC3A, APLNR, CACNB2, CLEC10A, CD300C, CREB3L3, STRA6, TRIM13, UBE2J1, SLC47A1, SYT13, PCSK5, SLC25A17, RAB17, ENPP1, SLC27A2, SLCO1B3, SLCO1B1, SLC27A5, SLC19A3, PEX11G, SLC18A2, KSR2, RNF144B, LIN7A, KCNQ4, SYNGR4, MITD1, PRRT2, NAT8, MYORG, MCOLN1, KIRREL3, LRRN2, SLC25A15, OBSCN, LDLRAD3, AATK, UGT2B4, TMEM132A, TFR2, SLC16A14, TNS2, UNC13B, UGT2B15, SLC25A25, ST7L, RAB43, RUFY3, SLCO2B1, TEX261, ARHGAP21, NAPB, TSPAN2, TMEM219, SIGLEC9, VASN, TM4SF4, CRK, CA2, GGT1, GSDMB, GPR37, CYP2W1, CYP2D6, BMPR1A, C ACNA2D1, CACNA1A, FYN, FPR2, GAS2, SLC2A2, CHDH, HTR1A, AKR1A1, CYP2A7, FOLH1, CYP1A2, CYP3A4, CYP2A6, CYP2B6, FMO3, DEPDC5, MALL, KCNMA1, GPRC5B, NINJ1, CYP2C8, LRRC37B, EVA1B, FES, KMO, HSD17B2, MAL2, CYP2C19, BCL2L10, AQP9, ABCG8, AP1S2, CDHR2, CRB3, ANPEP, ASGR1, ADRA2B, ATP1A2, CYP2A13, GRAMD1A, ASIC5, ACAD11, BBS1, ASPHD1, HSD11B1, ASGR2, ATG2A, PIK3AP1, CYP3A43, ALDH3A2, B3GAT1, INTS2, CYP2C9, IBTK, MADCAM1, MYZAP, MFAP3L, LIMS2, NDUFS1, KCND1, GPR15, NIPAL4, IGFLR1, SLC2A8, RPS6KC1, LZTR1, HPS6, OR10R2, ABCC2, DPP4, LRRK2, SLC10A1, NPC1L1,
   e. Tissue protected: Kidney (glomeruli) ZAP70, CACNB2, ILK, MGAT4C, UBE2J1, SLC22A15, SLC47A1, MAPT, SYT13, SLC25A17, RAB17, SLC45A1, SH3BP4, SLC19A3, MGAM, KIRREL1, P2RX6, PILRA, IL10RB, KSR2, PARVB, PTPRG, MRGPRF, TPSG1, PTGDR, PLA2R1, PTH2R, F2R, KIRREL3, MAP6, NOS3, EHD2, MPP5, NKD1, LDLRAD3, NPHS2, RMC1, TSPAN7, SLC16A14, TNS2, PRRG2, SNCA, PSD4, SNX6, THSD7A, PITPNM3, PTPRO, SREBF1, SCN7A, NAPB, VASN, PRKCZ, EHD1, AIF1L, CDH6, FYN, GAS2, CD93, FAM107A, ADGRF1, CD34, FAM126B, HTR1A, CALN1, ENG, DOCK5, CR1, FGD2, KCNMA1, GPRC5B, EVA1B, MAS1, ADGRL4, CLIC2, AQP1, ABCG8, CLN3, CRB3, ACKR1, CHRM1, CDH13, ASPHD1, PAM, BBS7, B3GAT1, ABCC1, LIMS2, ITGA8, EHD3, NIPAL4, LZTR1, HPS6, NEU4, DYSF, DPP4,
   f. Tissue protected: Kidney (tubules) VNN1, SUN3, TRPM3, TBC1D3G, TREH, MS4A8, APLNR, CACNB2, ILK, MGAT4C, THY1, STRA6, UBE2J1, SLC22A15, SLC47A1, SELPLG, MAPT, TMEM240, PCSK5, RAB17, ENPP1, SMIM24, SLC5A12, SLC5A2, SLC12A1, PCDHB2, SLC27A2, SH3BP4, SCNN1D, S1PR2, SLC34A3, SLC19A3, MGAM, PEX11G, SLC28A2, RDH11, PTH1R, KIRREL1, P2RX6, VPS26B, IL10RB, PARVB, RHBDD2, DLC1, PDE2A, RALB, RNF144B, PSCA, LIN7A, MRGPRF, TRPM7, TMEM132E, PRICKLE1, SLCO4A1, STYK1, TMEM52B, KCNQ4, SLC9A3, IFI27, PTGDR, SGIP1, STXBP3, MITD1, NOS1, PEX10, PRRT2, OSBPL7, NAT8, MYORG, MCOLN1, KIRREL3, LRRN2, EHD2, MPP5, SLC25A15, OBSCN, LDLRAD3, AATK, RFTN2, UGT2B4, ATP6V1D, SLC22A6, TMEM72, TRPM2, UGT3A1, TMEM132A, VSIG10, ATP6V0A4, SLC7A7, UST, QTRT1, TMEM252, SLC16A14, TMEM174, TBC1D3, UNC13B, PLAUR, PRRG2, SLC28A1, SEMA6B, SLC22A2, RHOG, SCAMP5, RAB43, SLC22A8, SLC13A3, RUFY3, SNX6, SLC6A18, SLC13A2, TMEM213, SLC5A1, SLC4A4, SEC11C, TEX261, SREBF1, PIK3R5, PKHD1, ARHGAP21, SLC35G2, OPRD1, NAPB, TSPAN2, TMEM219, VASN, UBL3, TMEM39B, CRK, CA2, PRKCZ, GGT1, IYD, DPEP1, ENPP3, CUBN, CLSTN2, EHD1, GSDMB, DLK1, KIRREL2, CKMT2, CLTRN, SLC44A2, AIF1L, ADCY10, BMPR1A, CDH6, FAM151A, AP4M1, CPT1C, GAS2, FAM107A, FKBP2, SLC2A2, CHDH, CLCNKB, FAM126B, HTR1A, AKR1A1, ADGRG5, CLCNKA, ENOX2, FAM169A, TM7SF2, ATRAID, FOLH1, IL1RAP, CLIC4, FRRS1L, CYP2B6, CA4, FMO3, DEPDC5, GNG11, GLIPR1L2, KIAA0319, KCNMA1, GPRC5B, LRRC37B, FES, KMO, CPTP, MAL2, MAGI1, BCL2L10, CXCR1, AQP1, DSG1, ABCG8, CDC42EP5, BCL2, MPP1, HTR4, AP1S2, CDHR2, CRB3, ANK2, ANPEP, SLC7A9, CLDN10, DUSP15, FXYD2, ACE, CATIP, CLDN2, CPT1B, GRAMD1A, ASIC5, ACKR2, ANXA13, BBS1, CPNE2, ARHGEF4, AP4E1, ASPHD1, C12orf66, BLNK, PIK3AP1, C5AR1, CYP3A43, CDH16, CADM1, ALDH3A2, B3GAT1, INTS2, DIP2A, IBTK, LRFN2, MYZAP, KCNJ10, KCNJ12, MFAP3L, NDUFS1, ALOX15B, KCND1, MCUB, EHD3, MS4A4A, NIPAL4, IGFLR1, GAL3ST1, RPS6KC1, LRP2, LZTR1, HPS6, MFSD1, IL23R, OR10R2, MRC1, GRIN2D, NSMF, ABCC2, MDGA2, DYSF, DPP4, LRRK2, NPC1L1,
   g. Tissue protected: Heart (cardiomyocytes) TCTN2, SLC30A4, SPAG4, UCP3, EFNB3, APLNR, CACNB2, CD300C, CD36, ILK, MGAT4C, STRA6, TMEM106B, SLC22A15, SLC47A1, MAPT, SYT13, PCSK5, SLC25A17, RAB17, SLC19A3, PEX11G, BVES, P2RX6, VPS26B, IL10RB, PARVB, POPDC2, P2RY6, PLN, RNF144B, GPIHBP1, MRGPRF, SHISA4, INPP5D, SPATA9, SGCG, SLCO4A1, SRL, KCNQ4, SLC9A3, SGIP1, MITD1, MXRA7, PRRT2, OSBPL7, LANCL2, ART3, MYORG, KLHL41, MCOLN1, LRRN2, EHD2, SLC25A15, OBSCN, AATK, RFTN2, SIPA1, RYR2, TMEM132A, TMEM236, SLC7A7, YRDC, UST, TREML1, QTRT1, TBC1D3, PRRG2, SEMA6B, SLC25A25, SCAMP5, RAB27A, RAB11FIP4, RYR1, RUFY3, SNX6, SLCO2B1, SEMA5B, ST8SIA2, TEX261, SCN7A, ARHGAP21, SLC35G2, NAPB, TMEM219, VASN, UBL3, TMEM39B, CRK, EHD1, GSDMB, CKMT2, CYP2W1, BMPR1A, CDH6, AP4M1, CACNA2D1, CDH15, ADRA1A, CHDH, ADGRF1, FAM126B, HTR1A, CAVIN4, TM7SF2, HHATL, CALN1, CYB561A3, CHRNA1, IL1RAP, GPAT2, FGD2, CYP4X1, DEPDC5, MALL, CAMK2B, GPRC5B, LRRC37B, EVA1B, FES, CPTP, LEPROTL1, ADGRL4, CLIC2, CXCR1, FCGRT, ANKRD13B, B3GNT4, ABCG8, B3GNT8, ELOVL7, AP1S2, CD81, CDHR2, CLN3, ANK2, CNTNAP3, C3AR1, ATP1A2, CADPS, CPT1B, CDH13, CD1D, AKAP6, ADGRG6, ADRB1, ASIC5, ACAD11, BBS1, CPNE2, ARHGEF4, AP4E1, ASPHD1, BLNK, PAM, ALDH3A2, IBTK, MYZAP, MFAP3L, NCAM1, GPR182, LIMS2, NDUFS1, KCND1, MCUB, EHD3, GPR15, NIPAL4, IGFLR1, GAL3ST1, SLC2A8, IL18RAP, RPS6KC1, LZTR1, OR10R2, MCEMP1, DES, DYSF, LRRK2,
   h. Tissue protected: Thyroid TMEM158, TCTN2, SLC30A4, APLNR, CACNB2, ILK, TMEM100, STRA6, TMEM106B, TRIM13, UBE2J1, SLC22A15, SLC47A1, SYT13, PCSK5, SLC25A17, TMEM251, SLITRK4, RAB17, ENPP1, PCDHB2, SH3BP4, SCNN1D, SLC19A3, PEX11G, KCNQ5, BVES, TPO, VPS26B, IL10RB, RHBDD2, RNF144B, GPIHBP1, LIN7A, INPP5D, PRICKLE1, SLCO4A1, KCNQ4, IFI27, SYBU, STXBP3, MITD1, MXRA7, OSBPL7, LANCL2, PSD3, MCOLN1, LRRN2, SLC25A15, OBSCN, LDLRAD3, AATK, PORCN, TRPM2, SLC7A7, YRDC, UST, QTRT1, SLC16A14, TBC1D3, UNC13B, PRRG2, TNFRSF10C, RTP5, SLC25A25, RAB43, SLC26A4, RUFY3, SLCO2B1, TEX261, SREBF1, PIK3R5, ARHGAP21, SYN3, TMEM219, SIGLEC9, UBL3, TMEM39B, C19orf18, CSK, PRKCZ, IYD, EHD1, GPR37, CYP2W1, AIF1L, ADCY10, BMPR1A, CDH6, AP4M1, FYN, FPR2, FKBP2, CLCNKB, FAM126B, HTR1A, AKR1A1, CLCNKA, FCRL1, ATRAID, CALN1, IL1RAP, GPAT2, FGD2, FMO3, IPCEF1, CYP4X1, DEPDC5, MALL, CAMK2B, KCNMA1, EVA1B, FES, MAGI1, ADGRL4, CLIC2, CXCR1, ANKRD13B, ABCG8, BCL2, CRB3, ANK2, AQP4, C3AR1, CRHR1, ACKR2, BBS1, CPNE2, ARHGEF4, AP4E1, C12orf66, BLNK, C5AR1, CDH16, CEACAM3, CADM1, ALDH3A2, INTS2, AP3B2, IBTK, MYZAP, NCAM1, NDUFS1, GPR15, NIPAL4, IGFLR1, GAL3ST1, LZTR1, HPS6, MFSD1, OR10R2, GRIN2D, NCS1,
   i. Tissue protected: Pancreatic endocrine cells (Islets) SLC30A4, STXBP1, MAP1LC3A, CACNB2, SCGN, UBE2J1, MAPT, SYT13, S LC25A17, STX1A, RAB3A, RTN1, SCG3, POMGNT2, IL10RB, RAB3B, PRICKLE1, KCNQ4, SYBU, STXBP3, MITD1, MXRA7, PTPRN, LANCL2, PTH2R, MYORG, MCOLN1, KIRREL3, LRRN2, OBSCN, AATK, NECAB2, SLC17A6, ATP6V1D, TRPM2, SHISAL2B, SV2A, YRDC, TSPAN7, TNS2, TBC1D3, UNC13B, PLAUR, SLC30A8, PRNP, PRRG2, SLC35A3, SLC25A25, SCAMP5, RAB27A, PTPRN2, RUFY3, SLCO2B1, TEX261, MAPK10, ARHGAP21, SLC35G2, OPRD1, NAPB, TSPAN2, TMEM219, SIGLEC9, VASN, TMEM39B, CRK, PRKCZ, DLK1, FFAR2, DGCR2, CYP2W1, CLTRN, ADCY10, BMPR1A, CDH6, FKBP2, GNAO1, AKR1A1, FAM169A, FBXO2, CYP2B6, MAGI1, CXCR1, ABCG8, CD81, C3AR1, FXYD2, ATP1A2, CADPS, CRHR1, CD1D, ASIC5, BBS1, ARHGEF4, PAM, BBS7, CADM1, GAD2, ALDH3A2, IBTK, GNAZ, KCND1, IGFLR1, GAL3ST1, HPS6, OR10R2, MDGA2, DPP4, j. Tissue protected: Pancreatic exocrine cells TCTN2, CLEC10A, CD300C, ILK, TBC1D3D, TMEM100, STRA6, UBE2J1, SELPLG, SYT13, PCSK5, RAB17, ENPP1, SLC27A2, SH3BP4, SLC19A3, SEC16B, POMGNT2, VPS26B, IL10RB, RNF144B, PSCA, MRGPRF, SPATA9, TMEM132E, SLCO4A1, STYK1, IFI27, SYBU, STXBP3, MITD1, MXRA7, PTH2R, MYORG, MCOLN1, LRRN2, OBSCN, LDLRAD3, AATK, ATP6V1D, TMEM72, TRPM2, UGT3A1, VSIG10, YRDC, UST, SLC16A14, UNC13D, TBC1D3, UNC13B, PLAUR, TBC1D3E, TNFRSF10C, SYCN, SCAMP5, RAB27A, RUFY3, SLCO2B1, SLC4A4, SEC11C, PKHD1, SCN7A, ARHGAP21, ARHGAP27, NAPB, TMEM219, TRAF3IP3, TM4SF4, TMEM39B, CRK, CSK, CA2, PRKCZ, GGT1, DPEP1, CUZD1, GP2, KIRREL2, FA2H, ADCY10, BMPR1A, CDH6, AP4M1, FPR2, FKBP2, CHDH, ADGRF1, AKR1A1, FBXO2, GPAT2, FGD2, CA4, FMO3, CYP4X1, MALL, GPRC5B, LRRC37B, EVA1B, FES, MAGI1, LEPROTL1, AQP1, BCL2, HTR4, ELOVL7, AP1S2, CD81, CLN3, CRB3, ANPEP, CNTNAP3, CFTR, CLDN10, FXYD2, CADPS, CHST4, AP4E1, ASPHD1, C12orf66, PIK3AP1, ALG14, BBS7, CADM1, AQP12A, AQP12B, IBTK, ABCC1, LRFN5, MYZAP, NCAM1, NDUFS1, MCUB, IGFLR1, GAL3ST1, RPS6KC1, HPS6, OR10R2, LRRK2,
5. Cancer treated: head and neck cancer
   a. Tissue protected: Colon TRDN, AXL, ZDHHC2, SLC30A7, SFT2D2, UGT2A3, TECR, TMEM237, TMEM74B, TNFSF14, TTC17, MAP1LC3A, ATP8B2, CORO7, ACY3, CHST11, FIBCD1, FCAMR, GABARAPL2, MGAT4C, SLC46A1, SCGN, ST3GAL6, SLC14A1, TMEM106B, UBE2J1, ZGRF1, SLC47A1, PIM1, ALPL, STX1A, SNX24, SMIM24, SRI, SLC26A2, TNFRSF11A, NAAA, PITPNM1, AHCYL1, SFXN5, SOCS7, PEX1, PEMT, NDUFS8, NTRK3, RNF145, SLC19A3, PARD3, ARHGAP5, RHOF, PLPP1, NOXA1, BVES, RHOH, PIGR, PLEKHA3, MUC12, TPCN1, TULP3, TJP3, VPS26B, WSCD1, TCIRG1, WHAMM, PTPRD, KLRG1, PARVB, DLC1, PCDHA4, LMAN2, PIGU, PTPRT, RAP1GAP, PSTPIP2, RAB3B, PSCA, MFSD10, MRGPRF, SEL1L3, TMEM45A, SERP1, SCARA3, TPSG1, TRPM7, SLC18A1, SLCO4A1, SPPL3, STYK1, ITGA9, MAPKAP1, SLC9A3, IFI27, ITLN1, PTGDR, ADCYAP1R1, SYNGR4, NECAP1, PLIN2, PEX10, MRGPRX2, ABCC3, OSBPL7, CD46, ABCB1, PCDH11X, GPR183, LRRC55, SLC24A3, MACF1, MGST2, MGAT4A, LRRN2, CNEP1R1, DENND5B, SORD, OBSCN, AATK, MEGF8, PNKD, UGT2B4, SLC26A3, SERP2, TMEM72, TNFAIP8L3, SELENOT, SLC3A1, TMEM132A, UGT2B17, VSIG10, RNF112, SYT11, TMEM236, VPS28, SLC7A7, TMEM143, VPS35L, UGT2B28, ZDHHC8, ZP2, VAC14, SYTL4, SYTL3, TMEM128, TMEM64, TMEM253, TBCD, UBR4, TSPAN31, TMEM41A, TSPAN8, TNFRSF14, PLAUR, SYTL2, TMEM201, UGT2B15, SLC46A2, TSC2, MCUR1, PCDH1, SLC28A3, SCAMP5, PTAFR, RAB27A, SYP, SUSD6, PIGZ, PTPRN2, SLC39A14, SLC6A12, RUFY3, SLC6A15, SERINC1, RAB31, TRPM4, VTI1B, TNFRSF10A, TMEM164, STK38L, MTG2, PPM1L, SLC25A23, SEC11C, PIK3R5, PRRT3, IMMT, SLC35B1, ARHGAP27, NAPB, RNF186, TMEM219, TMEM154, TMEM171, VASN, TAS2R38, TCTN3, TRPC6, UBL3, TMEM39B, ADCK2, ATG2B, CA2, CCR10, CDHR5, CHST3, PRKCZ, GGT1, JAM3, DPEP1, GSDMB, FKRP, FCER1A, GPC4, IL9R, GHR, SLC2A12, CX3CR1, DPY19L4, FPR1, SLC48A1, CLEC16A, FCER1G, GOLPH3, DMXL2, ABCG2, AIF1L, ARL8A, ACSL5, CLCA1, CYSLTR1, GLP2R, CEACAM1, B3GNT5, FPR2, CADM4, FAR2, CHDH, GNB5, ADGRF1, RASGRP2, HTR1A, TESC, CLN6, EPHA4, ENOX2, CTNND1, DMTN, FLNC, FXYD6, ARL8B, AQP8, CNTFR, ADGRE1, ELOVL1, CHODL, ANKRD13A, CA4, CD1E, SLC37A4, DEPDC5, DPP10, HRH1, GNG11, DGKQ, GPR82, KCNMA1, IGSF11, LPAR6, CTDNEP1, EVA1B, CPTP, GJA4, HSD17B2, GALNT16, MAGI1, FPGS, MAN1C1, AP3M1, FADS3, ABHD16A, CXCR1, FER1L6, ANO8, ANKRD13B, AQP1, BEST2, BRI3, CDC42EP5, BCL2, MPP1, HTR4, B3GNT8, UGCG, KDELR3, ELOVL7, AP1S2, CDHR2, CLN3, CRB3 , DENND5A, ACE2, ANPEP, ATP9A, CNTNAP3, CDH17, ENPEP, SLC31A1, C3AR1, ALG2, SLC25A31, ATP11C, CLDN11, KCNIP3, ATP1A2, ACKR3, CD63, CLDND1, CERS6, CD300A, CYP20A1, GRAMD1A, ARRDC1, ADGRG6, ASIC5, B3GNT6, CEACAM6, TMEM30B, ACKR2, ANXA13, BBS1, CHST5, ARHGEF4, CMTM8, EPB41L3, ABHD12, AGPS, BCL2L13, CNPPD1, EBP, B3GNT7, HSD3B7, PAM, ALG14, CASD1, CHMP2B, CEACAM3, ABCD3, B3GAT1, CASQ1, HHLA2, CYP2C9, DIP2A, IBTK, ABCC5, SMPD4, HMOX1, SLC9A3R2, GRB14, MPZL1, NCEH1, LRFN5, MYZAP, NBAS, LHFPL2, MS4A18, GIPC1, MC4R, MS4A12, GFRA2, GLUL, MCUB, MTCH2, SLC2A10, SLC2A6, KIAA2013, NIPAL1, NIPAL4, APOOL, SLC16A10, IL17RA, LY9, GAL3ST1, GPA33, DPY19L1, HIGD1A, LZTR1, ALOX12, MUC13, NFXL1, HPS6, MYOC, MT-ND4, POMGNT1, ICA1, MFSD2A, NEU4, OR10R2, SLC16A7, MRC1, KLRC3, MUC4, PDLIM4, GPR63, IL4R, PSKH1,
   b. Tissue protected: Lung TGM2, AXL, SLC30A7, SLC30A4, SFT2D2, TMEM74B, EFNB3, CD300C, CHRM3, CHST11, ST3GAL6, SLC14A1, TRIM13, UBE2J1, ZGRF1, RNF144A, RNF145, SLC19A3, PIGS, BVES, AGER, PIGR, TJP3, SLC18A2, WHAMM, PTPRT, REEP2, RAP1GAP, PTPRG, MFSD10, SCARA3, NECAP1, MRGPRX2, OSBPL7, CD46, GPR183, MACF1, OBSCN, CD84, TMEM80, UBR4, PTPRB, TMEM201, PCDH1, RHOG, PSD4, SUSD6, RUFY3, SLC6A15, VTI1B, TMEM164, STEAP1, STK38L, PIK3R5, SIGLEC9, UBL3, ATG2B, CA2, CCR10, HLA-DRB1, EHD4, FPR1, SLC48A1, CD247, FCER1G, CYB5A, SLC44A2, DMXL2, ARL8A, ACSL5, CLCN5, AOC3, ECEL1, CLDN18, GNB5, TESC, EPHA4, HCLS1, CTNND1, DMTN, ARL8B, ELOVL1, ANKRD13A, CTXN2, GPR82, KCNMA1, AIFM2, LPAR6, EVA1B, GJA4, GALNT16, AP3M1, CLIC2, CXCR1, ANKRD13B, AQP1, ABCA8, AIG1, ANK2, ELMO2, AQP4, ENPEP, C3AR1, CACNA2D2, ACE, AP3S1, CD63, CLDND1, CYP20A1, AP3S2, CEACAM6, CMTM8, BDKRB2, CACNA1C, CADM1, ABCD3, CXCR3, CAVIN2, IBTK, GRB14, MPZL1, NCEH1, LAMP3, MC4R, GK, APOOL, GALNT13, HPS6, MFSD2A, SLC16A7, NSMF, SLC34A2, IL4R, PSKH1, LRRK2,
   c. Tissue protected: Skin TMEM243, TRDN, ZDHHC2, SFT2D2, SUN3, TMEM237, TMEM74B, CLEC10A, CD300C, CHRM3, ACER3, CHST11, GABARAPL2, MGAT4C, ST3GAL6, PERP, SLC47A1, SOX10, ALPL, SLC6A1, PITPNM1, SFXN5, TMEM97, RNF145, PARD3, ARHGAP5, NEGR1, NTM, NOXA1, TULP3, VPS26B, TCIRG1, PIGU, REEP2, RAP1GAP, GPIHBP1, MFSD10, LTBR, TMEM45A, SERP1, TYROBP, STYK1, ITGA9, MAPKAP1, SLC9A3, IFI27, NECAP1, PLIN2, ABCC3, GPR183, KCNAB1, LRRC55, LAX1, MACF1, MGST2, F2R, MERTK, LRRN2, SORD, DMPK, OBSCN, AATK, PNKD, SERP2, SELENOT, RMC1, VPS28, SLC7A7, YRDC, TMEM260, ZP2, VAC14, SYTL3, TMEM128, TBCD, UBR4, TNFRSF14, UGT2B15, TSC2, SLC28A3, S1PR5, PTAFR, SUSD6, TRPM4, VTI1B, TMEM164, SLC19A1, PRRT3, NAPB, SIGLEC9, TMEM171, TAS2R38, TRPC6, TMEM39B, ADCK2, CCR10, CHST3, CYBA, FFAR2, GPC4, DPY19L4, FPR1, CLEC16A, CYP2W1, DMXL2, ARL8A, CDH6, CLCN5, CYSLTR1, GLP2R, ANO7, FYN, FPR2, GPX8, FAR2, TESC, EPHA4, GPR84, ENOX2, ATRAID, CTNND1, DMTN, FAT2, FXYD6, ARL8B, CD99, CD248, ELOVL1, CAPNS2, ANKRD13A, CTXN2, DGKQ, GSDMA, FGFBP1, CTDNEP1, EVA1B, EDNRB, GJA4, GALNT16, AP3M1, FADS3, B3GNT4, ABCA8, ADGRF4, BRI3, KDELR3, ELOVL7, CD81, ANK2, SLC25A31, MS4A1, CLDND1, CAV3, CD1D, ASPRV1, ASIC5, TMEM63B, DSG3, TMEM30B, ACKR2, COL13A1, AGPS, BCL2L13, CLEC2A, CNPPD1, HSD3B7, PAM, ABCD3, IBTK, ABCC5, MPZL1, MYZAP, NBAS, GIPC1, MC4R, MCUB, SLC2A10, NIPAL1, NIPAL4, APOOL, HIGD1A, ALOX12, CHL1, POMGNT1, SLC2A11, MFSD2A, OR10R2, NCS1, IL4R, PSKH1, TMEM243, TCTN2, SLC30A7, SLC30A4, TECR, APLNR, CORO7, FIBCD1, TRIM13, UBE2J1, ULBP3, TNFRSF11A, TYR, PEMT, PDGFC, PIGS, RHOH, TPCN1, TJP3, WIPI2, DLC1, PLN, PTPRT, SEL1L3, SCARA3, RASA4, SLCO4A1, OSBPL7, CD46, ICAM3, DENND5B, SLC25A15, TMEM80, TMEM143, TMEM64, DCT, TSPAN31, PRNP, TMEM201, ZC4H2, SNTB1, MCUR1, PCDH1, RHOG, RUFY3, SERINC1, RAB31, TNFRSF10A, MTG2, PMEL, SEMA5B, ARHGAP27, TMEM219, TCTN3, CNTN3, FCER1A, GSDMC, EHD4, CYB5A, AIF1L, B3GNT5, TM7SF2, CYB561A3, FCGR2B, CNTFR, GLMP, FAM210A, ADGRE1, GPAT2, DNER, FGD2, SLC37A4, HRH1, IGSF11, CPTP, CXCR1, PROCR, BCL2, B3GNT8, UGCG, ELMO2, ATP9A, SLC31A1, AP3S1, CERS6, AP3S2, BBS1, CPNE2, CMTM8, ATG2A, BDKRB2, B3GNT7, CACNA1C, ALG14, ADGRA1, CHMP2B, CAVIN2, MPP2, HAS3, NCEH1, LHFPL2, MUC21, GFRA2, IL17RA, KIT, LZTR1, MT-ND4, PDLIM4, GPR63, ,
   d. Tissue protected: Liver VNN1, TRDN, AXL, SLC30A7, SFT2D2, TECR, TNFSF14, TTC17, MAP1LC3A, APLNR, CLEC10A, CD300C, CHRM3, ACY3, CHST11, FCAMR, GABARAPL2, ST3GAL6, SLC14A1, TRIM13, UBE2J1, ZGRF1, SLC47A1, PIM1, ALPL, SLC6A4, SNX24, NAAA, RNF144A, SLC25A40, SFXN5, SLCO1B3, SLCO1B1, RDX, PEMT, NDUFS8, NTRK3, SLC25A43, SLC27A5, SLC19A3, PARD3, NEGR1, PIGS, NOXA1, RHOH, PLEKHA3, TPCN1, TJP3, SLC18A2, PTPRD, KSR2, PTGDR2, LMAN2, PIGU, PTPRT, PDZK1, RDH16, LIN7A, SEL1L3, TMEM45A, SERP1, SCARA3, RASA4, SPPL3, ITGA9, MAPKAP1, SYNGR4, NECAP1, PLIN2, MRGPRX2, CD46, ABCB1, GPR183, LRRC55, NAT8, PNPLA3, MACF1, MGST2, MERTK, LRRN2, DENND5B, SORD, SLC25A15, OBSCN, AATK, MEGF8, PNKD, UGT2B4, SERP2, SELENOT, SLC3A1, TMEM132A, YIPF1, TFR2, TMEM260, VPS35L, ZDHHC8, UGT1A6, SYTL4, TMEM128, TMEM64, TNS2, TBCD, UBR4, TSPAN31, TNFRSF14, PTPRB, TMEM201, UGT2B15, TSC2, SLC25A25, ST7L, PCDH1, PTAFR, SUSD6, SLC39A14, SLC6A12, RUFY3, SERINC1, VTI1B, TMEM164, SLCO2B1, PPM1L, IMMT, NAPB, TSPAN2, TMEM219, SIGLEC9, TMEM171, VASN, CA2, CCR10, CDHR5, GGT1, JAM3, GSDMB, FKRP, FCER1A, GPC4, EVA1A, GHR, CX3CR1, DPY19L4, SLC48A1, CLEC16A, CYP2W1, CYP2D6, CYB5A, DMXL2, GJA5, ACSL5, CEACAM1, B3GNT5, CACNA1A, FYN, FPR2, GAS2, CADM4, FAR2, SLC2A2, CHDH, RASGRP2, HTR1A, TESC, CYP2A7, EPHA4, FOLH1, CYP1A2, CTNND1, DMTN, FXYD6, ARL8B, CNTFR, CYP3A4, FAM210A, CYP2A6, ELOVL1, CD1E, SLC37A4, FMO3, CYP4V2, CTXN2, DEPDC5, KCNMA1, GPRC5B, ABCB4, NINJ1, CYP2C8, DHCR24, CDH2, CTDNEP1, EVA1B, GPAM, KMO, GJA4, HSD17B2, GALNT16, MAL2, MAN1C1, AP3M1, CYP2C19, FADS3, AQP9, ABCA8, AIG1, BRI3, UGCG, AP1S2, CDHR2, CRB3, ANPEP, ENPEP, ASGR1, SLC31A1, ADRA2B, ABCB11, ATP1A2, CMKLR1, CYP2A13, ACKR3, CD63, CLDND1, CYP20A1, CYP2E1, GRAMD1A, CTSL, ARRDC1, ASIC5, TMEM63B, ACAD11, TMEM30B, BBS1, CMTM8, EPB41L5, HSD11B1, AGPS, BACE2, CD320, CYTH2, EBP, ASGR2, ATG2A, PIK3AP1, CYP3A43, HSD3B7, CHMP2B, ABCD3, B3GAT1, CASQ1, INTS2, CAVIN2, CYP2C9, IBTK, SMPD4, MOXD1, SLC9A3R2, GRB14, MPZL1, MYZAP, NBAS, LHFPL2, MFAP3L, GIPC1, LIMS2, GLUL, MTCH2, GPR15, GK, SLC2A6, NIPAL1, NIPAL4, APOOL, IL17RA, DPY19L1, SLC2A8, HIGD1A, LZTR1, ALOX12, HPS6, POMGNT1, SLC2A11, ICA 1, MFSD2A, OR10R2, ABCC2, PDLIM4, GPR63, PSKH1, LRRK2, SLC10A1, NPC1L1,
   e. Tissue protected: Kidney (glomeruli) AXL, SLC30A7, SFT2D2, TECR, ZAP70, CORO7, ACER3, CHST11, MGAT4C, ST3GAL6, SLC14A1, UBE2J1, SLC47A1, MAPT, SLC45A1, RNF144A, SFXN5, PEMT, RNF145, SLC19A3, PDGFC, PLPP1, PLEKHA3, WHAMM, KSR2, PARVB, PTPRT, RAP1GAP, PTPRG, MRGPRF, SEL1L3, SERP1, SCARA3, TPSG1, PECAM1, PTGDR, ADCYAP1R1, NECAP1, PLA2R1, CD46, PTH2R, KCNAB1, LRRC55, PNPLA3, MACF1, F2R, MGAT4A, MAP6, NOS3, DENND5B, NPHS2, SERP2, RMC1, VPS28, VPS35L, VAC14, TSPAN7, TNS2, STK10, PCDH1, PTAFR, PSD4, SUSD6, SLC6A12, RAB31, TRPM4, VTI1B, TNFRSF10A, TMEM164, STK38L, THSD7A, PITPNM3, PTPRO, SREBF1, SLC35B1, RTL1, NAPB, TMEM171, VASN, PRKCZ, JAM3, EVA1A, HYAL2, CX3CR1, EHD4, DMXL2, AIF1L, CDH6, CEACAM1, FYN, GAS2, CD93, FAM107A, GNB5, ADGRF1, CD34, RASGRP2, HTR1A, CTNND1, DMTN, ENG, CD99, CD248, DOCK5, FAM210A, ADGRE1, CR1, FGD2, HRH1, GPR82, KCNMA1, GPRC5B, EVA1B, MAS1, GJA4, GALNT16, ADGRL4, CLIC2, FADS3, ANO8, AQP1, CLN3, CRB3, ACKR1, ELMO2, ENPEP, CHRM1, CLDND1, CERS6, CYP20A1, EPB41L5, CACNA1C, PAM, BBS7, B3GAT1, SLC9A3R2, GRB14, HAS3, MPZL1, NBAS, GIPC1, LIMS2, ITGA8, MLIP, NIPAL4, LZTR1, NFXL1, HPS6, MFSD2A, NEU4, SLC16A7, DYSF, PSKH1, PDGFRA,
   f. Tissue protected: Kidney (tubules) TMEM231, TMEM243, VNN1, TRDN, AXL, ZDHHC2, SLC30A7, SFT2D2, SUN3, TRPM3, TECR, TMEM237, TBC1D3G, TMEM74B, TREH, TNFSF14, TTC17, APLNR, ATP8B2, CORO7, CHRM3, ACER3, ACY3, CHST11, FIBCD1, FCAMR, GABARAPL2, MGAT4C, ST3GAL6, THY1, SLC14A1, UBE2J1, ZGRF1, SLC47A1, MAPT, TMEM240, PIM1, ALPL, SLC6A4, SMIM24, SLC5A12, SLC5A2, STIMATE, SLC12A1, NAAA, PCDHB2, PITPNM1, RNF144A, SLC25A40, AHCYL1, SFXN5, SLC4A10, SCNN1D, PEX1, RDX, PEMT, SLC6A19, SLC22A11, S1PR2, NDUFS8, SLC34A3, NTRK3, NAV3, SLC19A3, PARD3, ARHGAP5, SLC28A2, RHOF, RDH11, NEGR1, PTH1R, PDGFC, PIGS, PLPP1, NOXA1, AGER, RHOH, PIGR, TPCN1, TULP3, TJP3, VPS26B, WSCD1, WIPI2, TCIRG1, WHAMM, PTPRD, PARVB, RHBDD2, DLC1, PCDHA4, LMAN2, PDE9A, PIGU, PTPRT, RALB, RAP1GAP, PDZK1, PSCA, LIN7A, MRGPRF, SEL1L3, LTBR, TMEM45A, UCHL1, SERP1, SCARA3, TRPM7, RASA4, SLCO4A1, SPPL3, STYK1, ITGA9, MAPKAP1, SLC9A3, IFI27, PTGDR, AD CYAP1R1, NECAP1, PLIN2, NOS1, PEX10, ABCC3, OSBPL7, CD46, ABCB1, GPR183, KCNAB1, LRRC55, NAT8, PNPLA3, SLC24A3, PLCD4, MACF1, MGST2, MGAT4A, MERTK, ITPR1, LRRN2, CNEP1R1, NTRK2, DENND5B, SORD, SLC25A15, OBSCN, AATK, MEGF8, NKAIN4, PNKD, RFTN2, UGT2B4, ATP6V1D, SLC22A6, SERP2, TMEM72, TNFAIP8L3, SELENOT, UGT3A1, XPNPEP2, SLC3A1, TMEM132A, VSIG10, YIPF1, VPS28, ATP6V0A4, SLC7A7, TMEM80, TMEM260, VPS35L, ZDHHC8, ZP2, VAC14, UGT1A6, SYTL4, TMEM252, SYTL3, TMEM128, TMEM174, TMEM64, TBCD, UBR4, TSPAN31, TMEM41A, TSPAN8, TNFRSF14, PLAUR, PTPRB, SYTL2, TMEM201, TMPRSS2, SLC46A2, TSC2, SLC28A1, SEMA6B, SNTB1, SIRT2, MCUR1, SLC22A2, PCDH1, RHOG, SLC28A3, SCAMP5, PTAFR, SLC22A8, SLC13A3, SUSD6, PIGZ, SLC39A14, SLC6A12, RUFY3, RAB31, SLC6A18, SLC13A2, TMEM213, TRPM4, VTI1B, TNFRSF10A, TMEM164, SLC5A1, STK38L, MTG2, TMEM184A, SLC4A4, SLC25A23, SEC11C, TMEM178B, SREBF1, PIK3R5, PKHD1, PRRT3, IMMT, SLC35G2, OPRD1, NAPB, TSPAN2, TMEM219, TMEM171, VASN, TAS2R38, TSPAN16, UBL3, TMEM39B, ADCK2, ATG2B, CA2, CCR10, CDHR 5, CHST3, ENPP6, EMC4, PRKCZ, GGT1, JAM3, IYD, DPEP1, ENPP3, CUBN, CLSTN2, GSDMB, DLK1, FKRP, FCER1A, GPC4, EVA1A, GHR, HYAL2, GSDMC, KIRREL2, CX3CR1, DPY19L4, EHD4, SLC48A1, CLEC16A, FCER1G, CKMT2, CPNE6, CYB5A, JAKMIP1, CLTRN, SLC44A2, DMXL2, AIF1L, ARL8A, GJA5, ACSL5, CDH6, CLCN5, FAM151A, B3GNT5, GAS2, CADM4, FAR2, FAM107A, FNDC5, SLC2A2, CHDH, DOCK2, GNB5, CLCNKB, HTR1A, ADGRG5, TESC, CLCNKA, CLN6, EPHA4, ENOX2, FAM169A, TM7SF2, ATRAID, CASR, FOLH1, CTNND1, DMTN, FXYD6, ARL8B, CNTFR, CD248, CLIC4, FAM210A, ADGRE1, ELOVL1, FRRS1L, ANKRD13A, CA4, CD1E, SLC37A4, FMO3, CYP4V2, CTXN2, DEPDC5, HRH1, GNG11, GLIPR1L2, LPXN, DGKQ, GPR82, KIAA0319, KCNMA1, GPRC5B, IGSF11, DHCR24, LPAR6, CDH2, CTDNEP1, KMO, EDNRB, LRFN4, CPTP, GJA4, GALNT16, MAL2, MAGI1, FPGS, MAN1C1, AP3M1, EPHB1, FADS3, ABHD16A, BSND, CXCR1, ANO8, AQP6, AQP1, ABCA8, AIG1, BRI3, CDC42EP5, NPR3, BCL2, MPP1, HTR4, UGCG, AP1S2, CDC42SE1, CDHR2, CRB3, ANK2, DENND5A, ANPEP, ATP9A, ENPEP, SLC7A9, CLDN10, DUSP15, SLC31A1, ALG2, SLC25A31, ATP11C, FXYD2, ACE, AP3S1, CATIP, CMKLR1, ACKR3, CD63, CLDND1, CLDN2, CERS6, CAV3, CYP20A1, CPT1B, GRAMD1A, CTSL, ARRDC1, ASIC5, AP3S2, TMEM63B, ACKR2, ANXA13, BBS1, COL13A1, CPNE2, ARHGEF4, CMTM8, EPB41L3, EPB41L5, AGPS, EBP, PIK3AP1, CACNA1C, CYP3A43, HSD3B7, CDH16, CHMP2B, CADM1, ABCD3, B3GAT1, CASQ1, INTS2, DIP2A, IBTK, MPP2, ABCC5, SMPD4, HMOX1, LRFN2, MOXD1, GRB14, HAS3, MPZL1, NCEH1, MYZAP, NBAS, LHFPL2, KCNJ10, KCNJ12, MFAP3L, GIPC1, MC4R, GFRA2, KCNK5, MCUB, MTCH2, SLC2A10, GK, MS4A4A, MLIP, SLC2A6, KIAA2013, NIPAL1, NIPAL4, APOOL, SLC16A10, IL17RA, FMO1, GAL3ST1, DPY19L1, HIGD1A, LRP2, LZTR1, ALOX12, NFXL1, HPS6, MYOC, MT-ND4, MFSD1, CHL1, POMGNT1, SLC2A11, ICA1, IL23R, MFSD2A, OR10R2, SLC16A7, MRC1, GRIN2D, NSMF, ABCC2, KCTD8, PDLIM4, DYSF, GPR63, IL4R, PSKH1, LRRK2, NPC1L1,
   g. Tissue protected: Heart (cardiomyocytes) TGM2, AXL, TCTN2, SLC30A7, SLC30A4, SLC27A6, SPAG4, TECR, TMEM237, TMEM74B, TNFSF14, TTC17, EFNB3, APLNR, ATP8B2, CAP2, CD300C, CHST11, FIBCD1, GABARAPL2, MGAT4C, SLC46A1, RASL10B, ST3GAL6, SLC14A1, TMEM106B, SLC47A1, MAPT, SNX24, NAAA, PITPNM1, RNF144A, SFXN5, SLC4A10, PEX1, SGCB, PEMT, NDUFS8, NTRK3, RNF145, SLC19A3, NEGR1, PIGS, BVES, RHOH, PLEKHA3, TPCN1, TJP3, VPS26B, WSCD1, TCIRG1, WHAMM, PTPRD, PARVB, POPDC2, P2RY6, PDE9A, PLCH2, PLN, RAP1GAP, GPIHBP1, MFSD10, MRGPRF, SEL1L3, SERP1, SCARA3, SHISA4, INPP5D, RASA4, SGCG, SLCO4A1, SRL, MAPKAP1, SLC9A3, ABCC3, OSBPL7, CD46, GPR183, LRRC55, ART3, SLC24A3, KLHL41, MACF1, MGST2, MGAT4A, MERTK, ITPR1, LRRN2, CNEP1R1, DENND5B, DMPK, SLC25A15, OBSCN, AATK, MEGF8, PNKD, RFTN2, SERP2, SIPA1, SELENOT, TMEM132A, YIPF1, TMEM236, SLC7A7, YRDC, TMEM143, TREML1, VPS35L, ZP2, VAC14, TMEM64, TBCD, UBR4, TSPAN31, TMEM41A, TNFRSF14, PTPRB, TMEM201, ZC4H2, TSC2, SEMA6B, SLC25A25, SNTB1, SIRT2, PCDH1, SCAMP5, PTAFR, RAB27A, RAB11FIP4, SUSD6, PIGZ, SLC39A14, SLC6A12, RYR1, SLAMF8, RUFY3, SERINC1, RAB31, TRPM4, VTI1B, TNFRSF10A, TMEM164, SLCO2B1, STK38L, MTG2, PPM1L, SLC25A23, TMEM178B, SEMA5B, ST8SIA2, IMMT, SLC35B1, SLC35G2, NAPB, TMEM219, TMEM171, VASN, TAS2R38, TCTN3, TRPC6, UBL3, TMEM39B, ATG2B, CCR10, JAM3, CNTN3, GSDMB, FKRP, FCER1A, GPC4, EVA1A, MMP27, CX3CR1, DPY19L4, EHD4, FPR1, CLEC16A, CKMT2, CYP2W1, GOLPH3, DMXL2, ABCG2, CDH6, CDH15, ADRA1A, B3GNT5, CADM4, GPX8, FAR2, FNDC5, CHDH, GNB5, ADGRF1, RASGRP2, HTR1A, CAVIN4, CLN6, SLC2A4, EPHA4, GPR84, TM7SF2, HHATL, CYB561A3, CTNND1, FCGR2B, FLNC, FXYD6, CHRNA1, ARL8B, CNTFR, CD248, ELOVL1, GPAT2, FGD2, ANKRD13A, CD1E, SLC37A4, CYP4V2, DEPDC5, HRH1, DGKQ, GPR82, CAMK2B, GPRC5B, IGSF11, CDH2, CTDNEP1, EVA1B, MBOAT7, CPTP, GJA4, GALNT 16, LEPROTL1, MAN1C1, ADGRL4, AP3M1, CLIC2, FADS3, ABHD16A, CXCR1, FCGRT, ANO8, ANKRD13B, B3GNT4, AIG1, B3GNT8, UGCG, ELOVL7, AP1S2, CDC42SE1, CD81, CDHR2, CLN3, ANK2, DENND5A, CNTNAP3, SLC31A1, CSF3R, C3AR1, ALG2, SLC25A31, ATP11C, AP3S1, ATP1A2, CMKLR1, ACKR3, CD63, CLDND1, CAV3, CD300A, CPT1B, CD1D, ARRDC1, AKAP6, ADGRG6, ADRB1, ASIC5, AP3S2, ACAD11, TMEM30B, BBS1, COL13A1, CPNE2, ARHGEF4, CMTM8, BCL2L13, BACE2, CNPPD1, CACNA1C, HSD3B7, PAM, ABCD3, CASQ1, IBTK, ABCC5, SMPD4, GRB14, MPZL1, NCEH1, MYZAP, NBAS, LHFPL2, MFAP3L, NCAM1, GIPC1, GPR182, LIMS2, MC4R, KCNK5, MCUB, MTCH2, SLC2A10, GPR15, GK, MLIP, NIPAL4, APOOL, SLC16A10, GAL3ST1, DPY19L1, SLC2A8, HIGD1A, IL18RAP, LZTR1, ALOX12, NFXL1, MT-ND4, POMGNT1, ICA1, OR10R2, MCEMP1, PDLIM4, DES, DYSF, GPR63, IL4R, PSKH1, LRRK2,
   h. Tissue protected: Thyroid TCTN2, SLC30A7, SLC30A4, SLC27A6, SFT2D2, TECR, TMEM237, TMEM74B, TNFSF14, TTC17, APLNR, ATP8B2, CHRM3, CHST11, FIBCD1, GABARAPL2, SLC46A1, ST3GAL6, TMEM100, TMEM106B, TRIM13, UBE2J1, ZGRF1, VPS33B, SLC47A1, TMEM251, PIM1, ALPL, SLITRK4, SNX24, SRI, TNFRSF11A, NAAA, PCDHB2, RNF144A, AHCYL1, SFXN5, SCNN1D, PEX1, RDX, PEMT, NDUFS8, NTRK3, RNF145, SLC19A3, PARD3, ARHGAP5, NEGR1, KCNQ5, PIGS, NOXA1, BVES, TPO, PLEKHA3, TPCN1, TULP3, TJP3, VPS26B, WSCD1, WIPI2, TCIRG1, WHAMM, RHBDD2, LMAN2, PIGU, RAP1GAP, GPIHBP1, LIN7A, SEL1L3, SERP1, SCARA3, INPP5D, SLCO4A1, MAPKAP1, IFI27, NMUR2, ABCC3, OSBPL7, CD46, GPR183, KCNAB1, PSD3, LRRC55, SLC24A3, MACF1, MGAT4A, LRRN2, CNEP1R1, NTRK2, DENND5B, SORD, SLC25A15, OBSCN, AATK, MEGF8, PORCN, PNKD, SERP2, SELENOT, YIPF1, VPS28, SLC7A7, YRDC, TMEM80, TMEM260, VPS35L, ZDHHC8, VAC14, SYTL4, SYTL3, TMEM128, TBCD, UBR4, TSPAN31, TMEM41A, TNFRSF14, TNFRSF10C, ZC4H2, TSC2, RTP5, SLC25A25, MCUR1, PCDH1, PTAFR, SLC26A4, SUSD6, SLC39A14, RUFY3, SERINC1, RAB31, VTI1B, TNFRSF10A, TMEM164, RNFT2, SLCO2B1, STK38L, MTG2, PPM1L, SLC25A23, SREBF1, PIK3R5, PRRT3, IMMT, SYN3, TMEM219, SIGLEC9, TMEM171, TCTN3, UBL3, TMEM39B, ADCK2, C19orf18, CCR10, CHST3, PRKCZ, JAM3, IYD, CNTN3, DUOX1, FCER1A, GPC4, EVA1A, GHR, CX3CR1, DPY19L4, EHD4, SLC48A1, CLEC16A, FCER1G, CYP2W1, GOLPH3, DMXL2, ABCG2, AIF1L, ARL8A, CDH6, B3GNT5, FYN, FPR2, CADM4, GPX8, FAR2, GNB5, CLCNKB, RASGRP2, HTR1A, TESC, CLCNKA, CLN6, EPHA4, ATRAID, CTNND1, DMTN, FCGR2B, FXYD6, ARL8B, CD99, CNTFR, GLMP, FAM210A, ADGRE1, ELOVL1, GPAT2, FGD2, ANKRD13A, CD1E, FMO3, IPCEF1, CTXN2, DEPDC5, DPP10, LPXN, DGKQ, CAMK2B, KCNMA1, IGSF11, LPAR6, CDH2, CTDNEP1, EVA1B, GJA4, GALNT16, MAGI1, ADGRL4, AP3M1, CLIC2, EPHB1, CXCR1, FER1L6, ANO8, ANKRD13B, PROCR, BCL2 , UGCG, CRB3, ANK2, DENND5A, ELMO2, AAK1, ATP9A, AQP4, ENPEP, C3AR1, ALG2, CMKLR1, CRHR1, ACKR3, CD63, CLDND1, CERS6, CD300A, CYP20A1, ARRDC1, TMEM63B, TMEM30B, ACKR2, BBS1, COL13A1, CPNE2, ARHGEF4, CMTM8, AGPS, CD320, CYTH2, CACNA1C, HSD3B7, CDH16, CDH10, CHMP2B, CEACAM3, CADM1, ABCD3, ARFGAP3, INTS2, CAVIN2, IBTK, MPP2, SMPD4, MPZL1, NCEH1, MYZAP, NBAS, LHFPL2, LRP1B, NCAM1, GIPC1, MC4R, GFRA2, GLUL, KCNK5, SLC2A10, GPR15, GK, SLC2A6, NIPAL4, APOOL, IL17RA, GAL3ST1, LZTR1, HPS6, MT-ND4, MFSD1, POMGNT1, SLC2A11, ICA1, MFSD2A, OR10R2, GRIN2D, NCS1, PDLIM4, GPR63, PSKH1,
   i. Tissue protected: Pancreatic endocrine cells (Islets) ZDHHC2, SLC30A4, SFT2D2, TNFSF14, TTC17, MAP1LC3A, CHRM3, ACER3, CHST11, FIBCD1, SCGN, UBE2J1, MAPT, PIM1, ALPL, STX1A, NAAA, RAB3A, PITPNM1, RNF144A, AHCYL1, RTN1, PEMT, NDUFS8, RNF145, SCG3, PARD3, ARHGAP5, NEGR1, PIGS, NOXA1, POMGNT2, TPCN1, TJP3, WSCD1, WIPI2, PTGDR2, PDE9A, PTPRT, RAP1GAP, RAB3B, UCHL1, SERP1, SCARA3, SPPL3, MAPKAP1, PTPRN, ABCC3, CD46, PTH2R, GPR183, LRRC55, MGST2, MGAT4A, LRRN2, CNEP1R1, OBSCN, AATK, NECAB2, PNKD, SLC17A6, ATP6V1D, SERP2, SHISAL2B, SV2A, YRDC, ZDHHC8, VAC14, SYTL4, TSPAN7, TMEM64, TNS2, UBR4, TSPAN31, TNFRSF14, PLAUR, SLC30A8, PRNP, TSC2, SLC25A25, STK10, PCDH1, SCAMP5, RAB27A, SYP, PTPRN2, RUFY3, RAB31, TRPM4, VTI1B, SLCO2B1, PPM1L, MAPK10, PRRT3, IMMT, SLC35G2, OPRD1, NAPB, TSPAN2, TMEM219, SIGLEC9, VASN, TMEM39B, ATG2B, CCR10, EMC4, PRKCZ, JAM3, DLK1, FFAR2, GPC4, DGCR2, CLEC16A, CYP2W1, GOLPH3, CLTRN, DMXL2, CDH6, CLCN5, B3GNT5, FAR2, GNB5, GNAO1, TESC, FAM169A, CASR, CTNND1, FXYD6, ARL8B, AQP8, CD99, CNTFR, ELOVL1, ANKRD13A, CYP4V2, DGKQ, IGSF11, LPAR6, CDH2, CTDNEP1, GJA4, MAGI1, CXCR1, UGCG, CD81, ENO2, ELMO2, ATP9A, CLU, C3AR1, ALG2, FXYD2, AMPH, ATP1A2, CRHR1, ACKR3, CD63, CLDND1, CYP20A1, CD1D, ARRDC1, ASIC5, TMEM30B, BBS1, ARHGEF4, EPB41L3, EPB41L5, CYTH2, PAM, BBS7, CADM1, GAD2, ABCD3, ARFGAP3, ALOX5, IBTK, MPP2, ABCC5, SMPD4, MPZL1, NBAS, GIPC1, MC4R, GFRA2, GLUL, MTCH2, SLC2A10, KIAA2013, APOOL, FMO1, GAL3ST1, HPS6, MT-ND4, ICA1, OR10R2, PDLIM4, PSKH1,
   j. Tissue protected: Pancreatic exocrine cells AXL, TCTN2, ZDHHC2, SLC30A7, SFT2D2, TMEM237, TNFSF14, ATP8B2, CLEC10A, CD300C, CORO7, CHRM3, CHST11, FIBCD1, GABARAPL2, SLC46A1, RASL10B, ST3GAL6, TBC1D3D, TMEM100, UBE2J1, ZGRF1, PIM1, ALPL, STIMATE, NAAA, AHCYL1, SFXN5, SLC4A10, PEX1, RDX, PEMT, TMEM97, NTRK3, RNF145, SLC19A3, PARD3, PDGFC, PIGS, PLPP1, NOXA1, POMGNT2, RHOH, PLEKHA3, TJP3, VPS26B, WSCD1, TCIRG1, WHAMM, PCDHA4, LMAN2, PDE9A, PIGU, PTPRT, RAP1GAP, PSCA, MFSD10, MRGPRF, SEL1L3, SERP1, SCARA3, SLCO4A1, STYK1, ITGA9, IFI27, MRGPRX2, ABCC3, CD46, PTH2R, GPR183, KCNAB1, LRRC55, SLC24A3, MACF1, MGST2, MGAT4A, MERTK, LRRN2, CNEP1R1, DENND5B, SORD, OBSCN, AATK, MEGF8, PNKD, ATP6V1D, SERP2, TMEM72, TNFAIP8L3, SELENOT, UGT3A1, SLC3A1, VSIG10, YRDC, TMEM80, ZDHHC8, VAC14, SYTL4, UNC13D, TMEM128, TSPAN31, TMEM41A, TSPAN8, PLAUR, TBC1D3E, PTPRB, SYTL2, TMEM201, TMPRSS2, TNFRSF10C, ZC4H2, SNTB1, PCDH1, SYCN, SLC28A3, SCAMP5, RAB27A, SUSD6, SLC39A14, RUFY3, SERINC1, RAB31, VTI1B, SLCO2B1, MTG2, SLC4A4, SLC25A23, SEC11C, SLC19A1, TMEM178B, PKHD1, IMMT, ARHGAP27, NAPB, TMEM219, TAS2R38, TCTN3, TMEM39B, ADCK2, CA2, CHST3, PRKCZ, GGT1, DPEP1, CUZD1, GPC4, EVA1A, GHR, GP2, SLC2A12, KIRREL2, DPY19L4, SLC48A1, CLEC16A, FCER1G, CYB5A, GOLPH3, DMXL2, ARL8A, ACSL5, CDH6, CYSLTR1, B3GNT5, CYSTM1, FPR2, FAR2, CHDH, ADGRF1, RASGRP2, TESC, CLN6, CTNND1, FCGR2B, FXYD6, A RL8B, AQP8, CNTFR, CD248, FAM210A, ELOVL1, GPAT2, FGD2, ANKRD13A, CA4, SLC37A4, FMO3, CYP4V2, DGKQ, GPR82, GPRC5B, IGSF11, LPAR6, CTDNEP1, EVA1B, GJA4, GALNT16, MAGI1, FPGS, LEPROTL1, MAN1C1, AP3M1, FADS3, AQP1, BRI3, BCL2, HTR4, KDELR3, CDH9, ELOVL7, AP1S2, CDC42SE1, CD81, CLN3, CRB3, DENND5A, ACE2, ANPEP, CNTNAP3, CFTR, CLDN10, ALG2, SLC25A31, ATP11C, FXYD2, CMKLR1, ACKR3, CD63, CLDND1, AQP5, CD300A, CYP20A1, CTSL, ARRDC1, TMEM30B, CHST4, COL13A1, CMTM8, AGPS, CYTH2, PIK3AP1, B3GNT7, HSD3B7, ALG14, BBS7, CHMP2B, CADM1, ABCD3, ARFGAP3, CASQ1, AQP12A, AQP12B, CAVIN2, IBTK, MPP2, SMPD4, MOXD1, GRB14, MPZL1, NCEH1, LRFN5, MYZAP, NBAS, NCAM1, GIPC1, MC4R, GFRA2, MCUB, MTCH2, SLC2A10, SLC2A6, KIAA2013, APOOL, SLC16A10, IL17RA, GAL3ST1, NFXL1, HPS6, MYOC, MT-ND4, POMGNT1, SLC2A11, ICA1, MFSD2A, OR10R2, SLC16A7, PDLIM4, PSKH1, LRRK2,
6. Cancer treated: colorectal cancer
   a. Tissue protected: Colon TMEM138, CD36, SCGN, ST3GAL6, UBE2J1, SNTG2, SYT13, PIM1, ALPL, STX1A, TMEM263, ENPP1, PITPNM1, SLC27A2, SOCS7, SEMA5A, SLC26A1, BVES, WHAMM, PTPRD, RAB27B, ITGB1, KLRG1, DLC1, PCDHA4, RECK, RAB3B, PSCA, KCNH8, MRGPRF, ADIPOR2, TRPM7, UPK3BL2, SLC18A1, MAPKAP1, ITLN1, SYNGR4, TAOK2, LRRC55, OBSCN, UGT2B4, SLC26A3, TMEM72, TMEM132A, SYT11, SLC7A7, TUB, ZP2, QTRT1, TMEM128, TMEM253, PLAUR, PRRG2, UPK3BL1, SEZ6, SCAMP5, SYP, SLC39A2, SLC6A12, RUFY3, SLC23A1, SLC35B1, PNLDC1, ARHGAP27, TMEM171, CA2, EFHD1, FKRP, IL9R, ABCG1, ABCG2, AIF1L, GPC5, CD177, GLP2R, B3GNT5, DCBLD1, EPHA4, GATM, ADGRA3, GJA1, CHODL, IGSF11, LRRC37B, HSD17B2, CDC42EP5, ANKFY1, SH3BP5, ANPEP, CNTNAP3, CLDN11, KCNIP3, CADPS, BIK, ARRDC1, DCXR, ACKR2, CMTM8, EPB41L3, COL6A2, CNPPD1, CASD1, B3GAT1, CYP2C9, DIP2A, HMOX1, S LC9A3R2, LRFN5, MYZAP, KCNN3, MS4A18, NDUFS1, MS4A12, GFRA2, IGFLR1, LZTR1, ALOX12, OR10R2, MRC1, KLRC3,
   b. Tissue protected: Lung SLC30A4, EFNB3, CD300C, CHRM3, ST3GAL6, TRIM13, UBE2J1, VIPR2, SLC22A15, TRPV4, SLC25A17, TGFBR3, RNF144A, SEMA4F, SLC26A1, NUS1, BVES, AGER, TMEM43, SLC18A2, WHAMM, RECK, REEP2, PTPRG, KIRREL3, EHD2, OBSCN, CD84, TMEM80, QTRT1, PTPRB, PRRG2, TMEM255B, P2RY2, SEZ6, RHOG, PSD4, SLC39A2, RUFY3, STEAP1, RYK, PNLDC1, CA2, EHD1, GJB4, EHD4, CD247, CCR7, CYB5A, ABCG1, CAVIN1, GPC5, AOC3, CDH11, DCBLD1, EPHA4, EPHX1, HCLS1, GJA1, CCDC88A, FES, CLIC2, DGKZ, ABCA8, AIG1, APBB1IP, ANKFY1, FCAR, ELMO2, CAV1, AQP4, CYB561D1, CACNA2D2, ACE, ANXA1, CMTM8, COL6A2, CAVIN2, LAMP3, EHD3, GK, SLC34A2, LRRK2,
   c. Tissue protected: Skin TMEM243, SUN3, TMEM138, CLEC10A, CD300C, CHRM3, ACER3, ST3GAL6, VIPR2, SOX10, SNTG2, TRPV4, SLC25A17, TGFBR3, ALPL, TMEM263, ENPP1, TMPRSS11D, SLC6A1, PITPNM1, TMEM97, SEMA5A, SLC26A1, NEGR1, NUS1, SLC26A9, TMEM43, RAB27B, MTMR2, REEP2, KCNH8, GPIHBP1, ADIPOR2, LTBR, TYROBP, MAPKAP1, TAOK2, LANCL2, LRRC55, LYPD3, KRT1, F2R, PLA2G4E, DMPK, OBSCN, RMC1, TMEM79, SLC7A7, TUB, YRDC, ZP2, TMEM128, THBD, PRRG2, TMEM255B, P2RY2, SLC39A2, NGFR, RYK, SLC19A1, PNLDC1, RIPK4, TMEM171, CLCA2, COL17A1, GJB4, FFAR2, FA2H, ABCG1, CAVIN1, GPC5, CDH6, CD177, GLP2R, ANO7, CDH11, DCBLD1, GPX8, EPHA4, GATM, ATRAID, FAT2, ADGRA3, CD99, CD248, GJA1, CAPNS2, GSDMA, LRRC37B, B3GNT4, DSG1, ABCA8, ADGRF4, ANKFY1, CD81, BST1, CAV1, CCR2, MS4A1, BIK, CD1D, ANXA1, ASPRV1, TMEM63B, DSG3, DCXR, ACKR2, CNPPD1, ATP12A, MYZAP, TACR1, ALOX12, CHL1, SLC2A11, OR10R2, NCS1, TMEM243, TCTN2, SLC30A4, ZDHHC6, TRIM13, UBE2J1, SYT13, ULBP3, TYR, RHBG, DLC1, PLN, KCNQ4, SLC25A15, TMEM80, QTRT1, DCT, PRNP, RHCG, SH2D3C, RHOG, RUFY3, PMEL, ARHGAP27, EHD1, GSDMC, EHD4, IGSF3, CYB5A, AIF1L, B3GNT5, ABHD17C, CYB561A3, FCGR2B, FAM210A, GPAT2, CD82, DNER, FGD2, IGSF11, DGKZ, BASP1, ELMO2, CYB561D1, CDH13, CMTM8, ADGRA1, CAVIN2, MPP2, MUC21, NDUFS1, GFRA2, EHD3, KIT, LZTR1, KIAA0040, ,
   d. Tissue protected: Liver SLC13A5, TMEM138, CLEC10A, CD300C, CHRM3, ST3GAL6, TRIM13, UBE2J1, SYT13, SLC25A17, PIM1, ALPL, SLC6A4, ENPP1, SYNE3, RNF144A, SLC25A40, SLC27A2, SLCO1B3, SLCO1B1, SEMA4F, SLC27A5, SEMA5A, SLC26A1, NEGR1, SLC18A2, PTPRD, RAB27B, KSR2, MTMR2, PTGDR2, RECK, RDH16, LIN7A, KCNQ4, MAPKAP1, SYNGR4, TAOK2, LRRC55, NAT8, KIRREL3, SLC25A15, OBSCN, UGT2B4, TMEM132A, YIPF1, TFR2, TUB, TMEM128, TNS2, ZDHHC13, PTPRB, ST7L, SLC39A2, SLC6A12, RUFY3, SLCO2B1, TEX261, TSPAN2, TMEM171, CA2, FKRP, EVA1A, GPR37, CYP2D6, CYB5A, ABCG1, GJA5, CD177, B3GNT5, CACNA1A, GAS2, SLC2A2, CREB3L2, CYP2A7, EPHA4, GATM, EPHX1, FOLH1, CYP1A2, ADGRA3, CYP3A4, FAM210A, CYP2A6, CYP2B6, FMO3, GPRC5B, NINJ1, CYP2C8, LRRC37B, CDH2, FES, KMO, HSD17B2, MAL2, CYP2C19, BCL2L10, AQP9, ABCA8, AIG1, ANKFY1, ANPEP, ASGR1, CYB561D1, ADRA2B, ABCB11, CYP2A13, CTSL, ARRDC1, TMEM63B, DCXR, CMTM8, BACE2, ASGR2, PIK3AP1, B3GAT1, CAVIN2, CYP2C9, SLC9A3R2, MADCAM1, MYZAP, MFAP3L, LIMS2, NDUFS1, GPR15, GK, IGFLR1, SLC2A8, LZTR1, ALOX12, SLC2A11, OR10R2, LRRK2, GAA, NPC1L1,
   e. Tissue protected: Kidney (glomeruli) ZDHHC16, TMEM138, ZDHHC6, ACER3, ST3GAL6, UBE2J1, VIPR2, KDR, S LC22A15, MAPT, SYT13, TRPV4, SLC25A17, TGFBR3, SYNE3, SLC45A1, RNF144A, PODXL, KIRREL1, TMEM43, WHAMM, ITGB1, KSR2, RECK, PTPRG, MRGPRF, PECAM1, SCUBE1, PLA2R1, PTH2R, LRRC55, F2R, KIRREL3, EHD2, NKD1, NPHS2, RMC1, TUB, TSPAN7, TNS2, PRRG2, TMEM255B, SNCA, PSD4, SLC6A12, THSD7A, RYK, PITPNM3, PTPRO, SLC35B1, RTL1, TMEM171, EFHD1, EHD1, EVA1A, EHD4, AIF1L, CAVIN1, GPC5, CDH6, CDH11, DCBLD1, GAS2, CD93, FAM107A, CD34, ENG, CD99, CD248, GJA1, DOCK5, FAM210A, CR1, FGD2, GPRC5B, MAS1, ADGRL4, CLIC2, ANKFY1, ELMO2, CAV1, CHRM1, BIK, CDH13, ANXA1, COL6A2, BBS7, B3GAT1, SLC9A3R2, LIMS2, ITGA8, EHD3, LZTR1, KIAA0040, DYSF, PDGFRA,
   f. Tissue protected: Kidney (tubules) TMEM243, SUN3, TRPM3, TMEM138, ZDHHC6, TBC1D3G, TREH, CHRM3, ACER3, ST3GAL6, THY1, UBE2J1, VIPR2, SLC22A15, SELPLG, MAPT, TMEM240, TRPV4, PIM1, ALPL, SLC6A4, TMEM263, ENPP1, SYNE3, SLC5A12, SLC5A2, SLC12A1, PCDHB2, PITPNM1, RNF144A, SLC25A40, SLC27A2, SLC6A19, SEMA4F, S1PR2, SLC34A3, SEMA5A, SLC26A1, RDH11, NEGR1, PTH1R, KIRREL1, AGER, RHBG, WHAMM, PTPRD, RAB27B, DLC1, PCDHA4, PDE2A, RECK, RALB, PSCA, KCNH8, KCNJ1, LIN7A, MRGPRF, LTBR, UCHL1, TRPM7, UPK3BL2, TMEM132E, KCNQ4, MAPKAP1, NOS1, LRRC55, NAT8, KIRREL3, NTRK2, EHD2, SLC25A15, OBSCN, NKAIN4, RFTN2, UGT2B4, ATP6V1D, SLC22A6, TMEM72, UGT3A1, TMEM132A, YIPF1, ATP6V0A4, SLC7A7, TUB, TMEM80, ZP2, QTRT1, TMEM252, TMEM128, TMEM174, PLAUR, ZDHHC13, PTPRB, RHCG, PRRG2, TMEM255B, UPK3BL1, SLC28A1, SLC22A2, SH2D3C, RHOG, SCAMP5, SLC22A8, SLC39A2, SLC6A12, RUFY3, SLC6A18, SLC13A2, TMEM213, SLC4A4, SLC23A1, RYK, TEX261, SLC35G2, OPRD1, PNLDC1, RIPK4, TSPAN2, TMEM171, TSPAN16, CA2, ENPP6, EMC4, EFHD1, IYD, CUBN, CLSTN2, EHD1, DLK1, FKRP, EVA1A, GSDMC, KIRREL2, EHD4, CPNE6, CYB5A, ABCG1, CLTRN, AIF1L, GJA5, GPC5, CDH6, CD177, FAM151A, CPT1C, B3GNT5, DCBLD1, GAS2, FAM107A, FNDC5, SLC2A2, ABHD17C, ADGRG5, CALCR, CREB3L2, EPHA4, FAM169A, GATM, ATRAID, CASR, FOLH1, ADGRA3, CD248, CLIC4, GJA1, FAM210A, FRRS1L, CYP2B6, FMO3, GLIPR1L2, KIAA0319, GPRC5B, IGSF11, LRRC37B, CDH2, FES, KMO, LRFN4, MAL2, BSND, BCL2L10, DSG1, ABCA8, AIG1, CDC42EP5, NPR3, ANKFY1, CDC42SE1, SH3BP5, ANPEP, BST1, CCR2, CLDN10, FXYD2, ACE, CATIP, BIK, CPT1B, COX7B, CTSL, ARRDC1, TMEM63B, DCXR, ACKR2, CMTM8, EPB41L3, COL6A2, ATP12A, PIK3AP1, CDH16, B3GAT1, DIP2A, MPP2, HMOX1, LRFN2, MYZAP, KCNJ10, MFAP3L, NDUFS1, GFRA2, KCNK5, EHD3, GK, MS4A4A, IGFLR1, FMO1, LRP2, LZTR1, ALOX12, KIAA0040, CHL1, NXPE2, SLC2A11, OR10R2, MRC1, MDGA2, KCTD8, DYSF, LRRK2, NPC1L1,
   g. Tissue protected: Heart (cardiomyocytes) TCTN2, SLC30A4, SLC27A6, SPAG4, TMEM138, ZDHHC6, UCP3, EFNB3, CAP2, CD300C, CD36, RASL10B, ST3GAL6, VIPR2, SLC22A15, MAPT, SYT13, TRPV4, SLC25A17, TGFBR3, PITPNM1, RNF144A, SGCB, NEGR1, NUS1, BVES, TMEM43, WHAMM, PTPRD, P2RY6, RECK, PLCH2, PLN, GPIHBP1, MRGPRF, ADIPOR2, SHISA4, FLT1, SGCG, SRL, KCNQ4, MAPKAP1, LANCL2, LRRC55, ART3, DMPK, EHD2, SLC25A15, OBSCN, RFTN2, TMEM132A, YIPF1, SLC7A7, YRDC, TREML1, ZP2, QTRT1, ZDHHC13, PTPRB, PRRG2, SH2D3C, SCAMP5, SLC39A2, SLC6A12, RYR1, SLAMF8, RUFY3, SLCO2B1, TEX261, SLC35B1, SLC35G2, TMEM171, EHD1, FKRP, EVA1A, MMP27, EHD4, ABCG1, ABCG2, CAVIN1, GPC5, CDH6, CDH15, CDH11, ADRA1A, B3GNT5, DCBLD1, GPX8, FNDC5, ABHD17C, CREB3L2, EPHA4, HHATL, FAM168B, CYB561A3, FCGR2B, CHRNA1, ADGRA3, CD248, GJA1, GPAT2, FGD2, GPRC5B, IGSF11, LRRC37B, CDH2, FES, MBOAT7, LEPROTL1, ADGRL4, CLIC2, FCGRT, B3GNT4, DGKZ, AIG1, ANKFY1, DMD, CDC42SE1, CD81, CNTNAP3, BST1, CAV1, CCR2, CADPS, BIK, CPT1B, COX7B, CDH13, CD1D, ARRDC1, ADRB1, CFC1, DCXR, CMTM8, BACE2, CNPPD1, MYZAP, MFAP3L, NCAM1, LIMS2, NDUFS1, KCNK5, EHD3, GPR15, GK, IGFLR1, SLC2A8, LZTR1, ALOX12, OR10R2, MCEMP1, DES, DYSF, LRRK2,
   h. Tissue protected: Thyroid TCTN2, SLC30A4, SLC27A6, TMEM138, ZDHHC6, CHRM3, ST3GAL6, TMEM100, TRIM13, UBE2J1, VIPR2, VPS33B, SLC22A15, SYT13, TRPV4, SLC25A17, TGFBR3, TMEM251, PIM1, ALPL, TMEM263, ENPP1, PCDHB2, RNF144A, SEMA4F, SEMA5A, NEGR1, NUS1, BVES, TPO, TMEM43, WHAMM, GPIHBP1, LIN7A, ADIPOR2, UPK3BL2, KCNQ4, MAPKAP1, TAOK2, LANCL2, LRRC55, NTRK2, SLC25A15, OBSCN, PORCN, YIPF1, SLC7A7, TUB, YRDC, TMEM80, QTRT1, TMEM128, PRRG2, TMEM255B, UPK3BL1, RTP5, SLC39A2, RUFY3, SLCO2B1, RYK, TEX261, PNLDC1, TMEM171, C19orf18, EFHD1, IYD, DUOX1, EHD1, EVA1A, GPR37, EHD4, ABCG1, ABCG2, AIF1L, CAVIN1, GPC5, CDH6, B3GNT5, DCBLD1, GPX8, CREB3L2, EPHA4, GATM, FAM168B, ATRAID, FCGR2B, ADGRA3, CD99, GJA1, FAM210A, GPAT2, FGD2, FMO3, IPCEF1, IGSF11, CDH2, FES, ADGRL4, CLIC2, ANKFY1, BASP1, ELMO2, AAK1, CAV1, AQP4, CRHR1, BIK, ANXA1, ARRDC1, TMEM63B, DCXR, ACKR2, CMTM8, CDH16, ARFGAP3, CAVIN2, MPP2, MTNR1B, MYZAP, NCAM1, NDUFS1, GFRA2, KCNK5, GPR15, GK, IGFLR1, LZTR1, SLC2A11, OR10R2, NCS1, GAA,
   i. Tissue protected: Pancreatic endocrine cells (Islets) SLC30A4, CHRM3, ACER3, SCGN, UBE2J1, MAPT, SYT13, TRPV4, SLC25A17, TGFBR3, PIM1, ALPL, STX1A, RAB3A, PITPNM1, RNF144A, RTN1, SCG3, NEGR1, PTGDR2, RAB3B, ADIPOR2, UCHL1, UPK3BL2, KCNQ4, MAPKAP1, TAOK2, PTPRN, LANCL2, PTH2R, LRRC55, KIRREL3, OBSCN, NECAB2, SLC17A6, ATP6V1D, SV2A, TUB, YRDC, TSPAN7, TNS2, PLAUR, ZDHHC13, SLC30A8, PRNP, PRRG2, TMEM255B, UPK3BL1, SCAMP5, SYP, SLC39A2, RUFY3, SLCO2B1, TEX261, MAPK10, SLC35G2, OPRD1, TSPAN2, EMC4, DLK1, FFAR2, DGCR2, CLTRN, CDH6, B3GNT5, GNAO1, CREB3L2, FAM169A, CASR, ADGRA3, CD99, CD82, CYP2B6, IGSF11, CDH2, DGKZ, ANKFY1, CD81, ELMO2, FXYD2, AMPH, CADPS, CRHR1, CD1D, ARRDC1, CFC1, EPB41L3, COL6A2, BBS7, GAD2, ARFGAP3, MPP2, GFRA2, IGFLR1, FMO1, OR10R2, MDGA2, GAA,
   j. Tissue protected: Pancreatic exocrine cells TCTN2, TMEM138, CLEC10A, CD300C, CHRM3, RASL10B, ST3GAL6, TBC1D3D, TMEM100, UBE2J1, SELPLG, SYT13, PIM1, ALPL, TMEM263, ENPP1, SLC27A2, TMEM97, SLC26A1, TMEM43, WHAMM, RAB27B, PCDHA4, PSCA, KCNH8, MRGPRF, ADIPOR2, UPK3BL2, TMEM132E, SCUBE1, PTH2R, LRRC55, OBSCN, ATP6V1D, TMEM72, UGT3A1, YRDC, TMEM80, UNC13D, TMEM128, PLAUR, TBC1D3E, ZDHHC13, PTPRB, TMEM255B, UPK3BL1, SYCN, SCAMP5, SLC39A2, RUFY3, SLCO2B1, SLC4A4, RYK, SLC19A1, PNLDC1, ARHGAP27, CA2, EFHD1, CUZD1, EVA1A, KIRREL2, FA2H, CYB5A, ABCG1, CDH6, B3GNT5, CYSTM1, CREB3L2, GATM, EPHX1, FCGR2B, CD248, FAM210A, GPAT2, FGD2, FMO3, GPRC5B, IGSF11, LRRC37B, FES, LEPROTL1, CDC42SE1, CD81, ANPEP, CNTNAP3, CLDN10, FXYD2, CADPS, AQP5, BIK, CTSL, ARRDC1, CHST4, CMTM8, COL6A2, PIK3AP1, BBS7, ARFGAP3, AQP12A, AQP12B, CAVIN2, MPP2, LRFN5, MYZAP, NCAM1, NDUFS1, GFRA2, IGFLR1, SLC2A11, OR10R2, LRRK2, GAA,
7. Cancer treated: liver cancer
   a. Tissue protected: Colon ZDHHC2, MS4A8, MGAT4C, SCGN, SLC14A1, UBE2J1, SNTG2, STX1A, SLC5A6, SLC26A2, SLC25A33, GOLGA8B, RNF145, MGAM, PILRA, MUC12, WHAMM, KLRG1, DLC1, PLCB3, RNF215, PSTPIP2, RAB3B, PSCA, KCNH8, TPSG1, TRPM7, UPK3BL2, SLC18A1, ITLN1, PTGDR, ADCYAP1R1, SYNGR4, OSBPL7, LRRC55, PLPP7, MAST2, SLC8A1, OBSCN, TMEM72, SLC3A1, SCARF1, XKR9, VSIG10, SYT11, VSTM5, ZP2, QTRT1, TMEM69, TMEM41A, UNC13B, PLAUR, UPK3BL1, SLC46A2, ARHGAP17, SEZ6, SCAMP5, RAB27A, RNF103, SYP, PTPRN2, SNX6, SEC11C, PHLPP2, TRAF3IP3, VTCN1, UBL3, ATG2B, C2CD2L, JAM3, DPEP1, GPBAR1, IGSF8, PHKA1, AIF1L, GPC5, CLCA1, CADM4, FAM126B, RASGRP2, DOCK8, ENOX2, CALN1, GOLGA8A, ADGRE1, CHODL, ANKRD13A, CARMIL2, EPCAM, CPTP, ABHD16A, CXCR1, FER1L6, BEST2, GAL3ST2, KDELR3, CLN3, SH3BP5, CLDN11, KCNIP3, ATP1A2, CADPS, B3GNT6, EPB41L3, ABHD12, COL6A2, BCL2L13, C12orf66, CNPPD1, PAM, ALG14, CASD1, B3GAT1, CASQ1, DIP2A, IBTK, ABCC5, LRFN5, MYZAP, IFFO1, MC4R, MS4A12, MARCKS, MCUB, GNG12, SLC16A10, LZTR1, MUC13, NFXL1, NEU4, OR10R2, MRC1, NDST2,
   b. Tissue protected: Lung VSIG4, SLC30A4, SLC14A1, TRIM13, UBE2J1, SLC22A15, TGFBR3, SLC6A14, RNF145, TRPV6, SLC18A2, WHAMM, REEP2, RNF215, PTPRG, MGAT4B, STX5, MXRA7, OSBPL7, MAST2, EHD2, OBSCN, VSTM5, QTRT1, P2RY2, SEZ6, RHOG, PSD4, RNF103, STEAP1, PHLPP2, UBL3, ATG2B, EHD1, GPBAR1, EHD4, CD247, CCR7, GPC5, ECEL1, CALN1, HCLS1, ANKRD13A, CCDC88A, CLIC2, CXCR1, DGKZ, ANK2, AQP4, ACE, BSN, AQP3, COL6A2, CACNA1C, IBTK, LAMP3, MC4R, MARCKS, EHD3, NSMF,
   c. Tissue protected: Skin TMEM243, ZDHHC2, SUN3, ACER3, MGAT4C, PERP, RXFP1, SOX10, SNTG2, TGFBR3, SLC5A6, SLC6A1, TMEM97, RNF145, MTMR2, REEP2, RNF215, KCNH8, LTBR, MGAT4B, TYROBP, STX5, SLC10A6, MXRA7, LANCL2, LRRC55, PLPP7, MAST2, LYPD3, PLA2G4E, SLC8A1, DMPK, OBSCN, RMC1, TMEM79, VSTM5, YRDC, ZP2, TMEM69, P2RY2, ARHGAP17, NGFR, SLC19A1, RIPK4, TRAF3IP3, C2CD2L, CLCA2, COL17A1, CYBA, IGSF8, FA2H, PHKA1, DSP, GPC5, CDH6, ANO7, FYN, GPX8, FAM126B, ENOX2, CALN1, FAT2, CD248, CAPNS2, ANKRD13A, CARMIL2, HCN1, GSDMA, B3GNT4, DSG1, KDELR3, ANK2, MS4A1, CD1D, ASPRV1, DSG3, AQP3, BCL2L13, CLEC2A, CNPPD1, ATP12A, PAM, IBTK, ABCC5, MYZAP, MC4R, MARCKS, MCUB, GNG12, TACR1, CHL1, OR10R2, NCS1, TMEM243, SLC30A4, ZDHHC6, MS4A8, SLC2A1, TRIM13, UBE2J1, SLC6A14, TYR, SLC25A33, GOLGA8B, PDGFC, RHBG, DLC1, PLN, RASA4, OSBPL7, XKR9, QTRT1, DCT, UNC13B, PRNP, SH2D3C, RHOG, S100A8, PMEL, SEMA5B, PHLPP2, VTCN1, UPK3A, EHD1, GSDMC, EHD4, AIF1L, ADCY7, HLA-DPA1, EVI2A, FCGR2B, GOLGA8A, ADGRE1, GPAT2, DNER, FGD2, CPTP , CXCR1, DGKZ, CDH13, CACNA1C, ALG14, ADGRA1, MPP2, LHFPL6, MUC21, IFFO1, EHD3, KIT, LZTR1, KIAA0040, ,
   d. Tissue protected: Liver SLC14A1, TRIM13, UBE2J1, SLC25A40, SLCO1B3, SLCO1B1, SLC25A33, SLC18A2, MTMR2, RNF215, MGAT4B, RASA4, STX5, SYNGR4, PRRT2, LRRC55, PNPLA3, MAST2, SLC8A1, OBSCN, SLC3A1, UNC13B, ARHGAP17, ST7L, JAM3, GPR37, GLRB, FYN, CADM4, RASGRP2, FOLH1, CARMIL2, NINJ1, ATP1A2, B3GAT1, CASQ1, IBTK, MYZAP, LIMS2, GPR15, LZTR1, OR10R2, NDST2,
   e. Tissue protected: Kidney (glomeruli) ZDHHC6, ACER3, MGAT4C, SLC14A1, UBE2J1, KDR, SLC22A15, TGFBR3, SLC45A1, RNF145, MGAM, PODXL, PDGFC, PILRA, TRPV6, WHAMM, RNF215, PTPRG, TPSG1, STX5, PTGDR, ADCYAP1R1, PLA2R1, PTH2R, LRRC55, PNPLA3, MAST2, MAP6, NOS3, EHD2, NPHS2, RMC1, SCARF1, VSTM5, TSPAN7, ARHGAP17, SNCA, PSD4, RNF103, SNX6, THSD7A, PITPNM3, PTPRO, SREBF1, RTL1, JAM3, EHD1, EHD4, AIF1L, GPC5, CDH6, FYN, CD93, FAM107A, CD34, FAM126B, RASGRP2, EVI2A, CALN1, ENG, CD248, DOCK8, ADGRE1, CR1, FGD2, MAS1, CLIC2, CLN3, CHRM1, BSN, CDH13, AKAP12, COL6A2, CACNA1C, PAM, B3GAT1, LIMS2, ITGA8, EHD3, GNG12, MLIP, LZTR1, NFXL1, KIAA0040, NEU4, PDGFRA,
   f. Tissue protected: Kidney (tubules) TMEM243, ZDHHC2, SUN3, ZDHHC6, TBC1D3G, TREH, MS4A8, ACER3, MGAT4C, THY1, SLC14A1, UBE2J1, SLC22A15, SELPLG, TMEM240, SLC5A12, SLC12A1, TLN2, PCDHB2, SLC25A40, S1PR2, SLC34A3, SLC25A33, GOLGA8B, MGAM, SLC28A2, RDH11, PTH1R, PDGFC, RHBG, WHAMM, RHBDD2, DLC1, PLCB3, PDE2A, RALB, RNF215, PSCA, KCNH8, LTBR, UCHL1, MGAT4B, TRPM7, UPK3BL2, RASA4, STX5, TMEM52B, PTGDR, ADCYAP1R1, NOS1, PRRT2, OSBPL7, LRRC55, PNPLA3, PLPP7, MAST2, SLC8A1, NTRK2, EHD2, OBSCN, RFTN2, SLC22A6, TMEM72, UGT3A1, SLC3A1, XKR9, VSIG10, ATP6V0A4, VSTM5, ZP2, QTRT1, TMEM252, TMEM174, TMEM41A, UNC13B, PLAUR, UPK3BL1, SLC46A2, ARHGAP17, SLC28A1, SEMA6B, SIRT2, SH2D3C, RHOG, SCAMP5, RNF103, SLC22A8, SNX6, SLC6A18, SLC13A2, SLC5A1, TMEM184A, SLC4A4, SEC11C, SREBF1, SLC35G2, RIPK4, VTCN1, TSPAN16, UPK3A, UBL3, ATG2B, ENPP6, EMC4, JAM3, IYD, DPEP1, ENPP3, CUBN, CLSTN2, EHD1, DLK1, GPBAR1, IGSF8, GLRB, GSDMC, KIRREL2, EHD4, PHKA1, AIF1L, GPC5, ADCY7, CDH6, CPT1C, CADM4, FAM107A, FNDC5, CLCNKB, FAM126B, ADGRG5, CLCNKA, DOCK8, ENOX2, EVI2A, CASR, FOLH1, GOLGA8A, CD248, CLIC4, ADGRE1, FRRS1L, ANKRD13A, CARMIL2, EPCAM, GLIPR1L2, KIAA0319, CPTP, EPHB1, ABHD16A, BSND, CXCR1, AQP6, DSG1, NPR3, CDC42SE1, ANK2, SH3BP5, SLC7A9, ACE, CATIP, BSN, CPT1B, AQP3, EPB41L3, COL6A2, C12orf66, ATP12A, C5AR1, CACNA1C, CDH16, B3GAT1, CASQ1, DIP2A, IBTK, MPP2, ABCC5, LRFN2, MYZAP, KCNJ10, KCNJ12, IFFO1, MC4R, MARCKS, MCUB, EHD3, GNG12, MLIP, SLC16A10, FMO1, LRP2, LZTR1, NFXL1, KIAA0040, MFSD1, CHL1, IL23R, OR10R2, MRC1, NSMF, NDST2,
   g. Tissue protected: Heart (cardiomyocytes) SLC30A4, SPAG4, ZDHHC6, UCP3, MGAT4C, RASL10B, SLC14A1, SLC22A15, TGFBR3, TLN2, SGCB, SLC25A33, RNF145, WHAMM, POPDC2, PLCH2, PLN, RNF215, SHISA4, MGAT4B, FLT1, RASA4, SGCG, SRL, MXRA7, PPP1R3A, PRRT2, OSBPL7, LANCL2, LRRC55, ART3, PLPP7, MAST2, SLC8A1, DMPK, EHD2, OBSCN, RFTN2, XKR9, YRDC, TREML1, ZP2, QTRT1, TMEM41A, ARHGAP17, SEMA6B, SIRT2, SH2D3C, SCAMP5, RAB27A, SLAMF8, SNX6, SEMA5B, SLC35G2, VTCN1, UPK3A, UBL3, ATG2B, JAM3, EHD1, MMP27, EHD4, PHKA1, DSP, GPC5, ADCY7, CDH6, CDH15, ADRA1A, CADM4, GPX8, FNDC5, FAM126B, RASGRP2, SLC2A4, HHATL, FAM168B, CALN1, FCGR2B, CHRNA1, CD248, GPAT2, FGD2, ANKRD13A, CARMIL2, CAMK2B, MBOAT7, CPTP, CLIC2, ABHD16A, CXCR1, FCGRT, B3GNT4, DGKZ, CDC42SE1, CLN3, ANK2, ATP1A3, ATP1A2, BSN, CADPS, CPT1B, CDH13, CD1D, AKAP6, BCL2L13, CNPPD1, CACNA1C, PAM, CASQ1, IBTK, ABCC5, MYZAP, NCAM1, GPR182, LIMS2, MC4R, MCUB, EHD3, GPR15, MLIP, SLC16A10, LZTR1, NFXL1, OR10R2, MCEMP1, DES, NDST2,
   h. Tissue protected: Thyroid SLC30A4, ZDHHC6, TRIM13, UBE2J1, VPS33B, SLC22A15, TGFBR3, TMEM251, SLITRK4, SLC5A5, SLC6A14, TLN2, PCDHB2, SLC25A33, RNF145, TPO, TRPV6, WHAMM, RHBDD2, RNF215, UPK3BL2, MXRA7, OSBPL7, LANCL2, PSD3, LRRC55, PLPP7, MAST2, SLC8A1, NTRK2, OBSCN, PORCN, XKR9, YRDC, QTRT1, TMEM41A, UNC13B, UPK3BL1, ARHGAP17, RTP5, RNF103, RNFT2, SREBF1, VTCN1, UBL3, C19orf18, C2CD2L, JAM3, IYD, DUOX1, EHD1, GPR37, EHD4, PHKA1, AIF1L, GPC5, CDH6, FYN, CADM4, GPX8, CLCNKB, FAM126B, RASGRP2, CLCNKA, FAM168B, CALN1, FCGR2B, ADGRE1, GPAT2, FGD2, ANKRD13A, CARMIL2, HCN1, IPCEF1, EPCAM, CAMK2B, CLIC2, EPHB1, CXCR1, FER1L6, ANK2, AAK1, AQP4, CRHR1, AKAP12, C12orf66, C5AR1, CACNA1C, CDH16, CDH10, AP3B2, IBTK, MPP2, MTNR1B, MYZAP, NCAM1, MC4R, GNG12, GPR15, LZTR1, MFSD1, OR10R2, NCS1, NDST2,
   i. Tissue protected: Pancreatic endocrine cells (Islets) ZDHHC2, SLC30A4, ACER3, SCGN, UBE2J1, TGFBR3, STX1A, RAB3A, RTN1, SLC25A33, RNF145, SCG3, RNF215, RAB3B, UCHL1, UPK3BL2, STX5, MXRA7, PTPRN, LANCL2, PTH2R, LRRC55, MAST2, SLC8A1, OBSCN, NECAB2, SLC17A6, XKR9, SV2A, YRDC, TMEM69, TSPAN7, UNC13B, PLAUR, SLC30A8, PRNP, UPK3BL1, SCAMP5, RAB27A, RNF103, SYP, PTPRN2, MAPK10, SLC35G2, ATG2B, C2CD2L, EMC4, JAM3, DLK1, DGCR2, PHKA1, CDH6, GNAO1, FBXO2, CASR, ANKRD13A, CARMIL2, EPCAM, CXCR1, DGKZ, ENO2, ATP1A2, CADPS, CRHR1, CD1D, EPB41L3, COL6A2, PAM, GAD2, IBTK, MPP2, ABCC5, MC4R, MARCKS, FMO1, OR10R2,
   j. Tissue protected: Pancreatic exocrine cells ZDHHC2, RASL10B, TBC1D3D, UBE2J1, SELPLG, TMEM97, SLC25A33, RNF145, PDGFC, WHAMM, RNF215, PSCA, KCNH8, UPK3BL2, STX5, MXRA7, PTH2R, LRRC55, PLPP7, MAST2, SLC8A1, OBSCN, TMEM72, UGT3A1, SLC3A1, VSIG10, VSTM5, YRDC, UNC13D, TMEM41A, UNC13B, PLAUR, TBC1D3E, UPK3BL1, ARHGAP17, SYCN, SCAMP5, RAB27A, RNF103, SLC4A4, SEC11C, SLC19A1, TRAF3IP3, DPEP1, CUZD1, GLRB, KIRREL2, FA2H, DSP, ADCY7, CDH6, RASGRP2, DOCK8, EVI2A, FBXO2, FCGR2B, CD248, GPAT2, FGD2, ANKRD13A, CARMIL2, EPCAM, KDELR3, CDC42SE1, CLN3, BSN, CADPS, CHST4, COL6A2, C12orf66, ALG14, CASQ1, AQP12A, AQP12B, IBTK, MPP2, LRFN5, MYZAP, NCAM1, IFFO1, MC4R, MARCKS, MCUB, GNG12, SLC16A10, NFXL1, OR10R2, NDST2,
8. Cancer treated: stomach cancer
   a. Tissue protected: Colon TMEM158, UGT2A3, TMEM138, TECR, MAP1LC3A, CACNB2, CD36, MGAT4C, SCGN, SLC14A1, TMEM245, TM4SF18, UBE2J1, SNTG2, SYT13, ALPL, STX1A, SUSD3, SLC26A2, TEX2, SLC27A2, SH3BP4, SCD5, RALGPS2, RNF145, SERINC3, SLC26A1, BVES, LDLR, P2RX6, PILRA, MUC12, TSPAN33, DLC1, PCDHA4, RECK, RRAD, PIGU, RAB3B, KCNH8, MRGPRF, TPSG1, UPK3BL2, SLC18A1, STYK1, TMEM134, ITGA9, PTGDR, ADCYAP1R1, SNX1, SYNGR4, SMPD2, NDUFAF5, OSBPL7, GNAI3, LRRC55, PALM, PLPP7, MSRA, MRC2, SORD, OBSCN, MFN2, SPRED1, UGT2B4, SLC26A3, TMEM72, SLC3A1, TMEM132A, UGT2B17, VSIG10, RNF112, SYT11, TMEM236, VSTM5, UTRN, UGT2B28, ZDHHC8, ZP2, QTRT1, TMEM69, SYTL3, TMEM128, TBCD, TMEM41A, PLAUR, ZFYVE9, UGT2B15, UPK3BL1, SCAMP5, SYP, SLC6A12, RUFY3, SNX6, TMEM204, SEC11C, PHLPP2, SLC35B1, TCTN3, ARHGEF12, ATG2B, C2CD2L, CHST3, GGT1, GNAI1, JAM3, HTR7, FMO5, IL9R, ABCG2, CD177, CLCA1, GLP2R, B3GNT5, FKBP2, GNB5, ADGRF1, FAM126B, HTR1A, ENOX2, FGFRL1, GABARAPL1, FMNL3, ADGRE1, CHODL, ANKRD13A, CARMIL2, CYP4X1, DEPDC5, DAG1, GBP2, LRRC37B, HSD17B2, ABHD16A, CXCR1, ANKRD13B, BEST2, ABCG8, CDC42EP5, BCL2, MPP1, GAL3ST2, CLN5, B3GALT5, CDHR2, CLN3, ACE2, SH3BP5, CNTNAP3, ANO9, SLC25A31, CLDN11, KCNIP3, ATP1A2, ATP2A2, ARRDC1, CIB1, ORAI1, ADGRG6, ASIC5, ACKR2, AP1S3, ASPHD1, EPB41L3, ABHD12, CNPPD1, ALG14, CASD1, DAPP1, B3GAT1, CYP2C9, DIP2A, IBTK, ABCC5, HMOX1, SLC9A3R2, LRFN5, SLC16A6, MYZAP, MS4A18, MS4A12, GFRA2, MCUB, JAML, NIPAL1, LZTR1, ALOX12, POMGNT1, NEU4, OR10R2, MRC1, PRKCA,
   b. Tissue protected: Lung TGM2, VSIG4, SLC30A4, EFNB3, CACNB2, CD300C, SLC14A1, TRIM13, TMEM245, UBE2J1, SLC22A15, TRPV4, SLC25A17, TGFBR3, SLC6A14, SUSD3, RNF144A, RNF145, SERINC3, SLC26A1, BVES, AGER, TSPAN33, TRPV6, SLC18A2, RECK, REEP2, PTPRG, MGAT4B, SNX1, SMPD2, OSBPL7, GNAI3, PALM, KIRREL3, MRC2, EHD2, OBSCN, MFN2, VSTM5, UTRN, QTRT1, TBC1D3, P2RY2, RHOG, PSD4, RUFY3, PHLPP2, SIGLEC9, ARHGEF12, ATG2B, GNAI1, EHD1, EHD4, CD247, CCR7, SLC44A2, AOC3, FKBP2, GNB5, FGFRL1, EPHX1, GABARAPL1, HCLS1, FMNL3, ANKRD13A, CCDC88A, DAG1, GBP2, AIFM2, CLEC2B, CLIC2, CXCR1, ANKRD13B, ABCA8, ABCG8, AIG1, ANK2, CAV1, AQP4, CACNA2D2, ACE, AP3S1, BSN, AP3S2, ASPHD1, CACNA1C, DAPP1, CXCR3, CAVIN2, IBTK, LAMP3, EHD3, PI4KA, SLC34A2, ICAM1, LRRK2,
   c. Tissue protected: Skin TMEM243, SUN3, TMEM138, CACNB2, CLEC10A, CD300C, MGAT4C, PERP, STRA6, TMEM245, RXFP1, SOX10, SNTG2, TRPV4, SLC25A17, TGFBR3, ALPL, TMPRSS11D, SLC6A1, RTN3, SH3BP4, TMEM97, RALGPS2, RNF145, SLC26A1, NEGR1, SLC26A9, MTMR2, PIGU, REEP2, KCNH8, GPIHBP1, MGAT4B, TYROBP, STYK1, TMEM134, ITGA9, PCDH19, SLC10A6, SNX1, SMPD2, LANCL2, GNAI3, LRRC55, PALM, PLPP7, LAX1, LYPD3, KRT1, F2R, PLA2G4E, MRC2, SORD, OBSCN, RMC1, TMEM79, VSTM5, YRDC, UTRN, ZP2, TMEM69, SYTL3, TMEM128, THBD, TBCD, TACSTD2, UGT2B15, P2RY2, S1PR5, NGFR, TMEM204, SIGLEC9, ARHGEF12, C2CD2L, CHST3, CLCA2, GNAI1, COL17A1, FFAR2, FA2H, CYP2W1, CD177, GLP2R, ANO7, FYN, FKBP2, FAM126B, GPR84, ENOX2, FGFRL1, GPR39, ATRAID, FAT2, CD99, CD248, FMNL3, CAPNS2, ANKRD13A, CARMIL2, MALL, DAG1, GBP2, DEF6, DSC3, LRRC37B, B3GNT4, DSG1, ABCA8, ABCG8, ADGRF4, CLN5, CD81, ANK2, BST1, CAV1, CCR2, ANO9, SLC25A31, MS4A1, CAV3, CD1D, ASPRV1, CIB1, ORAI1, ACSBG1, ASIC5, TMEM63B, DSG3, ACKR2, ASPHD1, CLEC2A, CNPPD1, ATP12A, DAPP1, IBTK, ABCC1, ABCC5, MYZAP, MCUB, FGFR3, NIPAL1, TACR1, ALOX12, CHL1, POMGNT1, PI4KA, SLC2A11, OR10R2, NCS1, TMEM243, TCTN2, SLC30A4, TECR, LRIG1, ARMCX2, SLC2A1, TRIM13, UBE2J1, SYT13, ULBP3, SLC6A14, SUSD3, TEX2, SCD5, RHBG, TSPAN33, WIPI2, DLC1, PLN, KCNQ4, TECTA, NDUFAF5, OSBPL7, ICAM3, MFN2, SPRED1, QTRT1, TBC1D3, DCT, PRNP, RHCG, SNTB1, SH2D3C, RHOG, S100A8, RUFY3, P MEL, SEMA5B, PHLPP2, TCTN3, UPK3A, DTL, EHD1, GSDMC, EHD4, ADCY7, HLA-DPA1, B3GNT5, EVI2A, FCGR2B, GLMP, FAM210A, ADGRE1, GPAT2, DNER, CYP4X1, CLEC2B, CXCR1, BCL2, BASP1, AP3S1, ATP2A2, CDH13, AP3S2, CACNA1C, ALG14, ADGRA1, CAVIN2, MPP2, LHFPL6, MUC21, GFRA2, EHD3, JAML, KIT, LZTR1, KIAA0040, ,
   d. Tissue protected: Liver SLC13A5, TMEM138, TECR, MAP1LC3A, CACNB2, CLEC10A, CD300C, SLC14A1, STRA6, TRIM13, TMEM245, TM4SF18, UBE2J1, SYT13, SLC25A17, ALPL, SLC6A4, SYNE3, SUSD3, RNF144A, SLC27A2, SLCO1B3, SCD5, SLCO1B1, RDX, SLC27A5, SERINC3, SLC26A1, NEGR1, LDLR, SLC18A2, KSR2, MTMR2, PTGDR2, RECK, RRAD, PIGU, PDZK1, RDH16, LIN7A, MGAT4B, ITGA9, KCNQ4, PCDH19, SNX1, SYNGR4, SMPD2, PRRT2, NDUFAF5, LRRC55, NAT8, MSRA, KIRREL3, SORD, OBSCN, SPRED1, UGT2B4, SLC3A1, TMEM132A, YIPF1, TFR2, UTRN, ZDHHC8, TMEM128, TNS2, TBCD, UGT2B15, SLC6A12, RUFY3, SLCO2B1, TMEM204, TEX261, TSPAN2, SIGLEC9, GGT1, JAM3, FMO5, GPR37, GLRB, CYP2W1, CYP2D6, GJA5, CD177, B3GNT5, FYN, GAS2, SLC2A2, HTR1A, CREB3L2, CYP2A7, HEPACAM, FGFRL1, EPHX1, GABARAPL1, FOLH1, CYP1A2, CYP3A4, FAM210A, FMNL3, CYP2A6, CARMIL2, FMO3, DEPDC5, MALL, GBP2, ABCB4, NINJ1, CYP2C8, LRRC37B, CDH2, CLEC2B, KMO, HSD17B2, MAL2, CYP2C19, BCL2L10, AQP9, ABCA8, ABCG8, AIG1, CLN5, CDHR2, ANO9, ASGR1, ADRA2B, ABCB11, ATP1A2, CMKLR1, CYP2A13, CTSL, ARRDC1, ASIC5, TMEM63B, ASPHD1, HSD11B1, BACE2, ASGR2, DAPP1, B3GAT1, INTS2, CAVIN2, CYP2C9, IBTK, SLC9A3R2, SLC16A6, MYZAP, MFAP3L, LIMS2, KCND1, GPR15, NIPAL1, LZTR1, ALOX12, POMGNT1, PI4KA, SLC2A11, OR10R2, ABCC2, LRRK2, GAA, NPC1L1,
   e. Tissue protected: Kidney (glomeruli) ZDHHC16, TMEM138, TECR, LRIG1, ARMCX2, CACNB2, MGAT4C, SLC14A1, TMEM245, TM4SF18, UBE2J1, KDR, SLC22A15, SYT13, TRPV4, SLC25A17, TGFBR3, SYNE3, SUSD3, SLC45A1, TEX2, RNF144A, SH3BP4, RALGPS2, RNF145, KIRREL1, P2RX6, PILRA, TSPAN33, TRPV6, KSR2, RECK, RRAD, PTPRG, MRGPRF, SLC34A1, TPSG1, SCUBE1, TECTA, PTGDR, ADCYAP1R1, SNX1, SMPD2, PLA2R1, NDUFAF5, PTH2R, GNAI3, LRRC55, PALM, F2R, KIRREL3, MAP6, MRC2, NOS3, EHD2, MPP5, NKD1, NPHS2, RMC1, VSTM5, UTRN, TSPAN7, TTC7B, TNS2, ZFYVE9, STK10, SNCA, PSD4, SLC6A12, SNX6, THSD7A, PITPNM3, PTPRO, SLC35B1, ARHGEF12, GNAI1, JAM3, HTR7, EHD1, EHD4, FYN, GAS2, CD93, FAM107A, GNB5, ADGRF1, CD34, FAM126B, HTR1A, EVI2A, GABARAPL1, ENG, CD99, CD248, DOCK8, FAM210A, FMNL3, ADGRE1, CR1, DAG1, GBP2, CLEC2B, MAS1, ADGRL4, CLIC2, ABCG8, B3GALT5, CLN3, ACKR1, CAV1, ANO9, CHRM1, BSN, CDH13, ASPHD1, CACNA1C, DAPP1, B3GAT1, ABCC1, SLC9A3R2, LIMS2, ITGA8, EHD3, MLIP, JAML, LZTR1, KIAA0040, PI4KA, NEU4, ICAM1, DYSF, PDGFRA,
   f. Tissue protected: Kidney (tubules) TMEM243, SUN3, TRPM3, TMEM138, TECR, TBC1D3G, TREH, ARMCX2, CACNB2, MGAT4C, THY1, SLC14A1, STRA6, TMEM245, TM4SF18, UBE2J1, SLC22A15, SELPLG, TMEM240, TRPV4, ALPL, SLC6A4, SYNE3, SLC5A12, SLC5A2, SUSD3, STIMATE, TEX2, SLC12A1, TLN2, PCDHB2, RNF144A, SLC27A2, SH3BP4, SCNN1D, SCD5, RDX, SLC6A19, S1PR2, SLC34A3, RALGPS2, SERINC3, SLC26A1, SLC28A2, RDH11, NEGR1, PTH1R, KIRREL1, LDLR, P2RX6, AGER, RHBG, WIPI2, RHBDD2, DLC1, PCDHA4, RECK, RRAD, PIGU, RALB, PDZK1, KCNH8, KCNJ1, LIN7A, MRGPRF, UCHL1, MGAT4B, SLC34A1, UPK3BL2, PRICKLE1, STYK1, TMEM134, TMEM52B, ITGA9, KCNQ4, PCDH19, PTGDR, ADCYAP1R1, SNX1, NOS1, SMPD2, PRRT2, NDUFAF5, OSBPL7, GNAI3, LRRC55, NAT8, PALM, PLPP7, MSRA, KIRREL3, MRC2, SORD, EHD2, MPP5, OBSCN, MFN2, RFTN2, SPRED1, UGT2B4, ATP6V1D, SLC22A6, TMEM72, UGT3A1, XPNPEP2, SLC3A1, TMEM132A, VSIG10, YIPF1, ATP6V0A4, VSTM5, UTRN, ZDHHC8, ZP2, QTRT1, TMEM252, SYTL3, TMEM128, TMEM174, TTC7B, TBC1D3, TBCD, TMEM41A, PLAUR, ZFYVE9, RHCG, UPK3BL1, SLC28A1, SEMA6B, SNTB1, SLC22A2, SH2D3C, RHOG, SCAMP5, SLC22A8, SLC6A12, RUFY3, SNX6, SLC6A18, SLC13A2, TMEM213, SLC5A1, SYT7, TMEM204, SLC4A4, SEC11C, TEX261, SLC35G2, OPRD1, TSPAN2, TSPAN16, UPK3A, ARHGEF12, ATG2B, CHST3, ENPP6, GGT1, GNAI1, JAM3, IYD, ENPP3, CUBN, CLSTN2, EHD1, DLK1, FMO5, GLRB, GSDMC, KIRREL2, EHD4, CKMT2, CPNE6, CLTRN, SLC44A2, GJA5, ADCY7, CD177, CPT1C, B3GNT5, GAS2, FAM107A, FKBP2, SLC2A2, GNB5, CLCNKB, FAM126B, HTR1A, ADGRG5, CALCR, CLCNKA, CREB3L2, ENOX2, EVI2A, FAM169A, FGFRL1, GABARAPL1, GPR39, ATRAID, CASR, FOLH1, CD248, CLIC4, FAM210A, ADGRE1, FRRS1L, ANKRD13A, CARMIL2, FMO3, DEPDC5, GLIPR1L2, LPXN, GBP2, KIAA0319, LRRC37B, CDH2, CLEC2B, KMO, LRFN4, MAL2, EPHB1, ABHD16A, BSND, BCL2L10, CXCR1, AQP6, DSG1, ABCA8, ABCG8, AIG1, CDC42EP5, NPR3, BCL2, MPP1, CLN5, CDC42SE1, CDHR2, ANK2, SH3BP5, BST1, CCR2, ANO9, SLC7A9, CLDN10, DUSP15, SLC25A31, FXYD2, ACE, AP3S1, CATIP, CMKLR1, ATP2A2, BSN, CAV3, CPT1B, COX7B, CTSL, ARRDC1, CIB1, ASIC5, AP3S2, TMEM63B, ACKR2, ANO5, AP1S3, ASPHD1, EPB41L3, ATP12A, C5AR1, CACNA1C, CDH16, B3GAT1, INTS2, DIP2A, IBTK, MPP2, ABCC5, HMOX1, LRFN2, SLC16A6, MYZAP, KCNJ10, KCNJ12, MFAP3L, GFRA2, KCND1, KCNK5, MCUB, EHD3, MLIP, JAML, NIPAL1, LRP2, LZTR1, ALOX12, KIAA0040, MFSD1, CHL1, POMGNT1, PI4KA, SLC2A11, IL23R, OR10R2, MRC1, ABCC2, LRRC24, MDGA2, PRKCA, DYSF, LRRK2, NPC1L1,
   g. Tissue protected: Heart (cardiomyocytes) TGM2, TCTN2, SLC30A4, SLC27A6, SPAG4, TMEM138, TECR, UCP3, EFNB3, ARMCX2, CAP2, CACNB2, CD300C, CD36, MGAT4C, SLC14A1, STRA6, TMEM245, SLC22A15, SYT13, TRPV4, SLC25A17, TGFBR3, SUSD3, TEX2, TLN2, RNF144A, RTN3, SCD5, SGCB, RALGPS2, RNF145, SERINC3, NEGR1, BVES, LDLR, P2RX6, TSPAN33, POPDC2, P2RY6, RECK, PLN, RRAD, GPIHBP1, MRGPRF, SHISA4, MGAT4B, SGCG, SRL, KCNQ4, PCDH19, TECTA, SNX1, SMPD2, PRRT2, NDUFAF5, OSBPL7, LANCL2, GNAI3, LRRC55, ART3, PLPP7, MSRA, MRC2, EHD2, OBSCN, MFN2, RFTN2, SPRED1, TMEM132A, YIPF1, TMEM236, YRDC, TREML1, UTRN, ZP2, QTRT1, TTC7B, TBC1D3, TBCD, TMEM41A, SEMA6B, SNTB1, SH2D3C, SCAMP5, SLC6A12, RYR1, SLAMF8, RUFY3, SNX6, SLCO2B1, TMEM204, SEMA5B, TEX261, SLC35B1, SLC35G2, TCTN3, UPK3A, ARHGEF12, ATG2B, GNAI1, JAM3, EHD1, EHD4, CKMT2, CYP2W1, ABCG2, ADCY7, CDH15, ADRA1A, B3GNT5, GNB5, ADGRF1, FAM126B, HTR1A, CAVIN4, CREB3L2, SLC2A4, GPR84, FGFRL1, HHATL, GABARAPL1, GPR39, FCGR2B, CHRNA1, CD248, FMNL3, GPAT2, ANKRD13A, CARMIL2, CYP4X1, DEPDC5, MALL, DAG1, GBP2, CAMK2B, LRRC37B, CDH2, CLEC2B, MBOAT7, LEPROTL1, ADGRL4, CLIC2, ABHD16A, CXCR1, FCGRT, ANKRD13B, B3GNT4, ABCG8, AIG1, CLN5, DMD, CDC42SE1, CD81, CDHR2, CLN3, ANK2, CNTNAP3, BST1, CAV1, CCR2, ANO9, CSF3R, SLC25A31, AP3S1, ATP1A2, CMKLR1, ATP2A2, BSN, CAV3, CPT1B, COX7B, CDH13, CD1D, ARRDC1, CIB1, ORAI1, AKAP6, ADGRG6, ADRB1, ASIC5, AP3S2, CFC1, ANO5, AP1S3, ASPHD1, BACE2, CNPPD1, CACNA1C, DAPP1, IBTK, ABCC5, SLC16A6, MYZAP, MFAP3L, NCAM1, GPR182, LIMS2, KCND1, KCNK5, MCUB, EHD3, GPR15, MLIP, IL18RAP, LZTR1, ALOX12, POMGNT1, PI4KA, OR10R2, LRRC24, MCEMP1, PRKCA, DES, DYSF, LRRK2,
   h. Tissue protected: Thyroid TMEM158, TCTN2, SLC30A4, SLC27A6, TMEM138, TECR, ARMCX2, CACNB2, TMEM100, STRA6, TRIM13, TMEM245, TM4SF18, UBE2J1, VPS33B, SLC22A15, SYT13, TRPV4, SLC25A17, TGFBR3, TMEM251, ALPL, SLITRK4, SLC6A14, TEX2, TLN2, PCDHB2, RNF144A, RTN3, SH3BP4, SCNN1D, SCD5, RDX, RNF145, SERINC3, NEGR1, BVES, TPO, TSPAN33, TRPV6, WIPI2, RHBDD2, RRAD, PIGU, GPIHBP1, LIN7A, UPK3BL2, PRICKLE1, KCNQ4, PCDH19, SNX1, SMPD2, OSBPL7, LANCL2, PSD3, GNAI3, LRRC55, PALM, PLPP7, MSRA, MRC2, SORD, OBSCN, MFN2, PORCN, SPRED1, YIPF1, YRDC, UTRN, ZDHHC8, QTRT1, SYTL3, TMEM128, TTC7B, TBC1D3, TBCD, TMEM41A, ZFYVE9, UPK3BL1, TNFRSF10C, RTP5, RUFY3, SLCO2B1, TMEM204, TEX261, SIGLEC9, TCTN3, ARHGEF12, C19orf18, C2CD2L, CHST3, GNAI1, JAM3, IYD, DUOX1, HTR7, EHD1, GPR37, EHD4, CYP2W1, ABCG2, B3GNT5, FYN, FKBP2, GNB5, CLCNKB, FAM126B, HTR1A, CLCNKA, CREB3L2, FGFRL1, GABARAPL1, ATRAID, FCGR2B, CD99, GLMP, FAM210A, FMNL3, ADGRE1, GPAT2, ANKRD13A, CARMIL2, FMO3, IPCEF1, CYP4X1, DEPDC5, MALL, DAG1, LPXN, CAMK2B, CDH2, ADGRL4, CLIC2, EPHB1, CXCR1, ANKRD13B, ABCG8, BCL2, CLN5, ANK2, BASP1, AAK1, CAV1, ANO9, AQP4, CMKLR1, ATP2A2, CRHR1, ARRDC1, CIB1, ORAI1, TMEM63B, ACKR2, AP1S3, C5AR1, CACNA1C, CDH16, CDH10, INTS2, AP3B2, CAVIN2, IBTK, MPP2, MTNR1B, MYZAP, NCAM1, GFRA2, KCNK5, GPR15, JAML, LZTR1, MFSD1, POMGNT1, PI4KA, SLC2A11, OR10R2, LRRC24, NCS1, PRKCA, GAA,
   i. Tissue protected: Pancreatic endocrine cells (Islets) SLC30A4, LRIG1, MAP1LC3A, ARMCX2, CACNB2, SCGN, TMEM245, UBE2J1, SYT13, TRPV4, SLC25A17, TGFBR3, ALPL, STX1A, TEX2, RNF144A, RTN1, RNF145, SCG3, NEGR1, POMGNT2, WIPI2, PTGDR2, RRAD, RAB3B, UCHL1, UPK3BL2, PRICKLE1, KCNQ4, SMPD2, PTPRN, LANCL2, PTH2R, GNAI3, LRRC55, KIRREL3, OBSCN, MFN2, NECAB2, SLC17A6, SPRED1, ATP6V1D, SV2A, YRDC, UTRN, ZDHHC8, TMEM69, TSPAN7, TTC7B, TNS2, TBC1D3, PLAUR, ZFYVE9, SLC30A8, PRNP, UPK3BL1, STK10, SCAMP5, SYP, RUFY3, SLCO2B1, SYT7, TMEM204, TEX261, MAPK10, SLC35G2, OPRD1, TSPAN2, SIGLEC9, ARHGEF12, ATG2B, C2CD2L, GNAI1, JAM3, HTR7, DLK1, FFAR2, FMO5, DGCR2, CYP2W1, CLTRN, B3GNT5, FKBP2, GNB5, GNAO1, CREB3L2, FAM169A, FGFRL1, CASR, CD99, FMNL3, ANKRD13A, CARMIL2, GBP2, CDH2, CXCR1, ABCG8, CLN5, CD81, ENO2, FXYD2, AMPH, ATP1A2, ATP2A2, CRHR1, CD1D, ARRDC1, ASIC5, CFC1, AP1S3, EPB41L3, GAD2, DAPP1, IBTK, MPP2, ABCC5, SLC16A6, GFRA2, KCND1, PI4KA, OR10R2, LRRC24, MDGA2, PRKCA, GPRIN1, GAA,
   j. Tissue protected: Pancreatic exocrine cells TCTN2, TMEM138, ARMCX2, CLEC10A, CD300C, TBC1D3D, TMEM100, STRA6, TMEM245, TM4SF18, UBE2J1, SELPLG, SYT13, ALPL, STIMATE, TEX2, SLC27A2, SH3BP4, SCD5, RDX, TMEM97, RNF145, SLC26A1, POMGNT2, TSPAN33, PCDHA4, RRAD, PIGU, KCNH8, MRGPRF, UPK3BL2, STYK1, ITGA9, SCUBE1, SNX1, SMPD2, PTH2R, GNAI3, LRRC55, PALM, PLPP7, MSRA, MRC2, SORD, OBSCN, SPRED1, ATP6V1D, TMEM72, UGT3A1, SLC3A1, VSIG10, VSTM5, YRDC, UTRN, ZDHHC8, UNC13D, TMEM128, TTC7B, TBC1D3, TMEM41A, PLAUR, TBC1D3E, ZFYVE9, TACSTD2, UPK3BL1, TNFRSF10C, SNTB1, SYCN, SCAMP5, RUFY3, SLCO2B1, SYT7, TMEM204, SLC4A4, SEC11C, TCTN3, ARHGEF12, CHST3, DTL, GGT1, GNAI1, CUZD1, GLRB, KIRREL2, FA2H, ADCY7, B3GNT5, CYSTM1, FKBP2, ADGRF1, CREB3L2, EVI2A, FGFRL1, EPHX1, FCGR2B, CD248, FAM210A, FMNL3, GPAT2, ANKRD13A, CARMIL2, FMO3, CYP4X1, MALL, DAG1, GBP2, LRRC37B, LEPROTL1, BCL2, CLN5, CDH9, CDC42SE1, CD81, CLN3, ACE2, CNTNAP3, CFTR, CLDN10, SLC25A31, FXYD2, CMKLR1, BSN, CTSL, ARRDC1, CIB1, CHST4, AP1S3, ASPHD1, ALG14, DAPP1, CAVIN2, IBTK, ABCC1, MPP2, LRFN5, MYZAP, NCAM1, GFRA2, MCUB, POMGNT1, SLC2A11, OR10R2, PRKCA, LRRK2, GAA,
9. Cancer treated: cervical cancer
   a. Tissue protected: Colon TRDN, TTYH3, TMED6, TMEM158, SCN4B, TMEM138, SLC7A6, DST, MAP1LC3A, CACNB2, CCND1, FCAMR, GABARAPL2, SCGN, SLC14A1, TMEM106B, UBE2J1, SLC47A1, SNTG2, SYT13, PCSK5, PIM1, ALPL, SLC22A16, STX1A, RAB17, SNX24, ENPP1, SMIM24, SLC38A9, SUSD3, PITPNM1, SLC27A2, SPIRE1, SIRPB1, MGAM, ARHGAP5, BVES, PILRA, PAG1, MUC12, TPCN1, PTPRD, ITGB1, KLRG1, DLC1, PLCB3, PCDHA4, RECK, SLC25A20, PVR, RNF144B, RAB3B, PSCA, KCNH8, MRGPRF, SEL1L3, TPSG1, TRPM7, SLC18A1, SLC25A36, SPATA9, STYK1, MAPKAP1, SLC9A3, ITLN1, PTGDR, ADCYAP1R1, PCDHB15, SNX7, SYNGR4, SLC11A1, MPC2, PEX10, MGLL, OSBPL7, ABCB1, PCDH11X, ITPR2, MFSD11, CNEP1R1, OBSCN, UGT2B4, TMEM72, SLC3A1, TMEM132A, VSIG10, TMEM236, SLC7A7, UST, ZP2, QTRT1, TMEM128, TMEM41A, PLAUR, ZFYVE9, RINT1, TMEM86A, SLC46A2, C2orf88, SPHK1, SCAMP5, RAB27A, SYP, PIGZ, PTPRN2, SLC6A12, RUFY3, SNX6, VTI1B, SLC38A5, SLC25A23, SEC11C, SUSD1, PHLPP2, ARHGAP21, REEP4, TMEM154, VASN, TCTN3, TRPC6, TMEM39B, ACVR1B, EFHD1, GGT1, DPEP1, FKRP, FCER1A, GREB1, SLC2A12, KCNMB3, CX3CR1, SLC48A1, CLEC16A, FCER1G, CLCA1, DAB2, DCBLD1, CADM4, ADGRF1, RASGRP2, CLN6, FGFRL1, CALN1, DMTN, FXYD6, ADGRA3, CHODL, CA4, CD1E, HHAT, DEPDC5, LRRC37B, CPTP, HSD17B2, MAGI1, MAN1C1, AP3M1, CDK14, ABHD16A, CXCR1, ANKRD13B, AQP1, CDC42EP5, MPP1, GAL3ST2, AP1S2, APOBR, CLN3, ACE2, SH3BP5, ANPEP, ATP9A, CNTNAP3, ENPEP, SLC25A31, CLDN11, KCNIP3, ADCK5, ATP1A2, CADPS, ASIC5, ACVRL1, ARL4C, ANXA13, BBS1, TMEM37, CHST5, ARHGEF4, EPB41L3, ABHD12, ADGRV1, ATP8B1, BCL2L13, EBP, ATG9A, PAM, ALG14, CASD1, B3GAT1, CYP2C9, DIP2A, IBTK, ABCC5, HMOX1, SLC9A3R2, NFASC, LRFN5, MYZAP, LFNG, LHFPL2, KCNN3, MS4A18, NDUFS1, GPR153, MS4A12, GFRA2, KIAA2013, NIPAL1, IGFLR1, IL17RA, LY9, GPA33, RPS6KC1, LZTR1, ALOX12, MUC13, NFXL1, MRAP, MT-ND4, NEU4, OR10R2, MRC1, NDUFB9, KLRC3, GPR63, NDST2,
   b. Tissue protected: Lung VSIG4, SLC30A4, SLC7A6, DST, EFNB3, CACNB2, SLC14A1, TRIM13, UBE2J1, SLC22A15, SLC25A17, TGFBR3, RAB17, SUSD3, RNF144A, SPIRE1, BVES, AGER, PAG1, VAT1, SLC18A2, IL10RB, RECK, SLC25A20, REEP2, RNF144B, PTPRG, SLC25A36, STX5, MPC2, MXRA7, MGLL, OSBPL7, ITPR2, KIRREL3, MFSD11, OBSCN, CD84, QTRT1, RINT1, TMEM86A, RHOG, PSD4, RUFY3, VTI1B, STEAP1, PHLPP2, REEP4, ACVR1B, GJB4, SLC48A1, CD247, FCER1G, CCR7, CYB5A, SLC44A2, DAB2, AOC3, DCBLD1, ECEL1, FGFRL1, CALN1, DMTN, CCDC88A, AIFM2, FES, AP3M1, CXCR1, ANKRD13B, AQP1, ABCA8, APBB1IP, FCAR, ELMO2, AQP4, ENPEP, CACNA2D2, ACE, ADCK5, BSN, CACNA1C, CXCR3, CAVIN2, IBTK, LAMP3, LFNG, GK, LDLRAD2, GALNT13, MRAP, PI4KA, NSMF, SLC34A2, LRRK2,
   c. Tissue protected: Skin TRDN, TTYH3, TMED6, SCN4B, SUN3, TMEM138, DST, CACNB2, CCND1, ACER3, GABARAPL2, SLC47A1, SOX10, SNTG2, SLC25A17, TGFBR3, ALPL, RAB17, ENPP1, SLC6A1, PITPNM1, SPIRE1, SIRPB1, TMEM97, ARHGAP5, NEGR1, SLC26A9, VAT1, IL10RB, REEP2, RNF144B, KCNH8, GPIHBP1, STX5, STYK1, MAPKAP1, SLC9A3, PCDHB15, SNX7, SLC11A1, MXRA7, MGLL, LANCL2, ITPR2, LAX1, F2R, DMPK, OBSCN, RMC1, SLC7A7, TMEM260, ZP2, TMEM128, TMEM86A, C2orf88, SPHK1, VTI1B, SLC38A5, SLC19A1, ARHGAP21, REEP4, TRPC6, TMEM39B, CYBA, GJB4, GABBR1, FA2H, CLEC16A, DSP, DAB2, CACNA2D1, DCBLD1, FYN, FGFRL1, CALN1, DMTN, FAT2, FXYD6, ADGRA3, CD99, CD248, HCN1, MALL, GSDMA, LRRC37B, AP3M1, CDK14, B3GNT4, DSG1, ABCA8, CLTCL1, APOBR, SLC25A31, ADCK5, MS4A1, CAV3, CD1D, ASPRV1, ASIC5, TMEM63B, ADGRV1, ATP8B1, BCL2L13, CLEC2A, ATP12A, PAM, IBTK, ABCC5, NFASC, MYZAP, LFNG, GPR153, NIPAL1, LDLRAD2, RPS6KC1, ALOX12, PI4KA, OR10R2, NDUFB9, NCS1, TRDN, TCTN2, SLC30A4, APLNR, TRIM13, UBE2J1, SYT13, SLC22A16, SLC38A9, SUSD3, TYR, TPCN1, DLC1, PLN, SEL1L3, KCNQ4, MPC2, OSBPL7, SLC25A15, UST, QTRT1, DCT, RINT1, RHOG, RUFY3, PMEL, SEMA5B, PHLPP2, TCTN3, FCER1A, GREB1, KCNMB3, IGSF3, CYB5A, EVI2A, CYB561A3, FCGR2B, GLMP, GPAT2, DNER, FGD2, CPTP, CXCR1, ELMO2, ATP9A, CDH13, BBS1, AP4E1, ATG2A, CACNA1C, ALG14, ADGRA1, CAVIN2, MPP2, LHFPL2, MUC21, NDUFS1, GFRA2, IL17RA, KIT, LZTR1, KIAA0040, MRAP, MT-ND4, GPR63, ,
   d. Tissue protected: Liver VNN1, TRDN, TTYH3, SLC13A5, SCN4B, TMEM138, DST, MAP1LC3A, APLNR, CACNB2, CCND1, CREB3L3, FCAMR, GABARAPL2, SLC14A1, TRIM13, UBE2J1, SLC47A1, SYT13, PCSK5, SLC25A17, PIM1, ALPL, SLC22A16, RAB17, SNX24, ENPP1, SYNE3, SLC38A9, SUSD3, RNF144A, SLC25A40, SLC27A2, SLCO1B3, SPIRE1, SLCO1B1, SIRPB1, SLC27A5, NEGR1, PAG1, TPCN1, VAT1, SLC18A2, PTPRD, RECK, SLC25A20, RNF144B, RDH16, LIN7A, SEL1L3, SLC25A36, STX5, KCNQ4, MAPKAP1, PCDHB15, SYNGR4, MPC2, PRRT2, MGLL, ABCB1, ITPR2, NAT8, PNPLA3, KIRREL3, MFSD11, MME, SLC25A15, OBSCN, UGT2B4, SLC3A1, TMEM132A, TFR2, TMEM260, TMEM128, TNS2, RINT1, TMEM86A, ST7L, C2orf88, SPHK1, SLC6A12, RUFY3, VTI1B, SLCO2B1, TEX261, ARHGAP21, TSPAN2, VASN, ACVR1B, GGT1, FKRP, FCER1A, EVA1A, GPR37, GREB1, KCNMB3, CX3CR1, SLC48A1, CLEC16A, CYP2D6, CYB5A, GJA5, CACNA2D1, CACNA1A, FYN, GAS2, CADM4, SLC2A2, RASGRP2, CREB3L2, CYP2A7, HEPACAM, FGFRL1, FOLH1, CYP1A2, DMTN, FXYD6, ADGRA3, CYP3A4, CYP2A6, CD1E, HHAT, DEPDC5, MALL, KCNJ16, ABCB4, NINJ1, CYP2C8, LRRC37B, CDH2, FES, GPAM, HSD17B2, MAL2, MAN1C1, AP3M1, CDK14, CYP2C19, BCL2L10, AQP9, ABCA8, AP1S2, ANPEP, ENPEP, ASGR1, ADRA2B, ABCB11, ADCK5, ATP1A2, CMKLR1, CYP2A13, ASIC5, TMEM63B, BBS1, HSD11B1, ADGRV1, ATP8B1, BACE2, EBP, ASGR2, ATG2A, PIK3AP1, CYP3A43, B3GAT1, INTS2, CAVIN2, CYP2C9, IBTK, SLC9A3R2, NFASC, MADCAM1, MYZAP, LFNG, LHFPL2, MFAP3L, LIMS2, NDUFS1, KCND1, GPR15, GK, NIPAL1, IGFLR1, IL17RA, LDLRAD2, SLC2A8, RPS6KC1, LZTR1, ALOX12, MRAP, PI4KA, OR10R2, NDUFB9, ABCC2, DPP4, GPR63, LRRK2, NDST2, SLC10A1, NPC1L1,
   e. Tissue protected: Kidney (glomeruli) SCN4B, TMEM138, DST, CACNB2, ACER3, SLC14A1, UBE2J1, SLC22A15, SLC47A1, MAPT, SYT13, SLC25A17, TGFBR3, RAB17, SYNE3, SUSD3, SLC45A1, RNF144A, SPIRE1, SIRPB1, MGAM, KIRREL1, PILRA, IL10RB, ITGB1, RECK, PVR, PTPRG, MRGPRF, SEL1L3, TPSG1, SLC25A36, STX5, SCUBE1, PTGDR, ADCYAP1R1, SLC11A1, MPC2, PLA2R1, MGLL, PTH2R, ITPR2, PNPLA3, F2R, KIRREL3, NOS3, MME, NKD1, NPHS2, RMC1, TSPAN7, TTC7B, TNS2, ZFYVE9, TMEM86A, C2orf88, STK10, SNCA, PSD4, SLC6A12, SNX6, VTI1B, SLC38A5, THSD7A, PITPNM3, PTPRO, SREBF1, VASN, EFHD1, EVA1A, HYAL2, CX3CR1, DAB2, DCBLD1, FYN, GAS2, CD93, FAM107A, ADGRF1, RASGRP2, EVI2A, CALN1, DMTN, ENG, CD99, CD248, DOCK8, CR1, FGD2, MAS1, ADGRL4, AQP1, CLN3, ELMO2, ENPEP, CHRM1, ADCK5, BSN, CDH13, ATP8B1, CACNA1C, PAM, BBS7, B3GAT1, SLC9A3R2, LIMS2, GPR153, ITGA8, LDLRAD2, LZTR1, NFXL1, NPHS1, KIAA0040, MRAP, PI4KA, NEU4, DYSF, DPP4, PDGFRA,
   f. Tissue protected: Kidney (tubules) TMEM231, VNN1, TRDN, TTYH3, TMED6, SCN4B, SUN3, TRPM3, TMEM138, TBC1D3G, TREH, DST, APLNR, CACNB2, CCND1, ACER3, FCAMR, GABARAPL2, THY1, SLC14A1, UBE2J1, SLC22A15, SLC47A1, MAPT, TMEM240, PCSK5, PIM1, ALPL, SLC22A16, RAB17, ENPP1, SMIM24, SYNE3, SLC5A12, SLC5A2, SUSD3, STIMATE, SLC12A1, PCDHB2, PITPNM1, RNF144A, SLC25A40, SLC27A2, SLC4A10, SCNN1D, SPIRE1, SIRPB1, SLC6A19, S1PR2, SLC34A3, NAV3, MGAM, ARHGAP5, SLC28A2, RDH11, NEGR1, PTH1R, KIRREL1, AGER, TPCN1, VAT1, VHL, PTPRD, IL10RB, RHBDD2, DLC1, PLCB3, PCDHA4, RECK, SLC25A20, PVR, RALB, RNF144B, PSCA, KCNH8, LIN7A, MRGPRF, SEL1L3, UCHL1, TRPM7, SLC25A36, STX5, STYK1, KCNQ4, MAPKAP1, SLC9A3, PTGDR, ADCYAP1R1, SNX7, SLC11A1, NOS1, MPC2, PEX10, PRRT2, MGLL, OSBPL7, ABCB1, ITPR2, NAT8, PNPLA3, PANX2, PLCD4, KIRREL3, MFSD11, CNEP1R1, NTRK2, MME, SLC25A15, OBSCN, RFTN2, UGT2B4, ATP6V1D, SLC22A6, TMEM72, XPNPEP2, SLC3A1, TMEM132A, VSIG10, ATP6V0A4, SLC7A7, TMEM260, UST, ZP2, QTRT1, TMEM252, TMEM128, TMEM174, TTC7B, TMEM41A, PLAUR, ZFYVE9, RINT1, TMPRSS2, TMEM86A, SLC46A2, SLC28A1, SEMA6B, SIRT2, SLC22A2, C2orf88, SPHK1, RHOG, SCAMP5, SLC22A8, PIGZ, SLC6A12, RUFY3, SNX6, SLC6A18, SLC13A2, TMEM213, VTI1B, SLC38A5, SLC5A1, SLC4A4, SLC25A23, SEC11C, TEX261, SREBF1, ARHGAP21, REEP4, SLC35G2, OPRD1, TSPAN2, VASN, TSPAN16, TMEM39B, ENPP6, EMC4, ACVR1B, EFHD1, GGT1, IYD, DPEP1, ENPP3, CUBN, CLSTN2, DLK1, FKRP, FCER1A, EVA1A, GABBR1, GREB1, HYAL2, KCNMB3, KIRREL2, CX3CR1, SLC48A1, CLEC16A, FCER1G, CKMT2, CPNE6, CYB5A, CLTRN, SLC44A2, GJA5, DAB2, CPT1C, DCBLD1, GAS2, CADM4, FAM107A, SLC2A2, CLCNKB, ADGRG5, CLCNKA, CLN6, CREB3L2, EVI2A, FAM169A, FGFRL1, CASR, FOLH1, DMTN, FXYD6, ADGRA3, CD248, CLIC4, FRRS1L, CA4, CD1E, HHAT, DEPDC5, GLIPR1L2, LPXN, KIAA0319, KCNJ16, LRRC37B, CDH2, FES, CPTP, MAL2, MAGI1, MAN1C1, AP3M1, CDK14, ABHD16A, BSND, BCL2L10, CXCR 1, AQP6, AQP1, DSG1, ABCA8, CDC42EP5, CLTCL1, NPR3, MPP1, AP1S2, CDC42SE1, SH3BP5, ANPEP, ATP9A, ENPEP, SLC7A9, CLDN10, SLC25A31, FXYD2, ACE, ADCK5, CATIP, CMKLR1, BSN, CAV3, CPT1B, ASIC5, TMEM63B, ACVRL1, ARL4C, ANO5, ANXA13, BBS1, TMEM37, ARHGEF4, AP4E1, EPB41L3, ADGRV1, ATP8B1, EBP, ATP12A, ATG9A, PIK3AP1, CACNA1C, CYP3A43, B3GAT1, INTS2, DIP2A, IBTK, MPP2, ABCC5, HMOX1, MYZAP, LFNG, LHFPL2, KCNJ10, KCNJ12, MFAP3L, NDUFS1, GFRA2, KCND1, KCNK5, GK, MS4A4A, KIAA2013, NIPAL1, IGFLR1, IL17RA, FMO1, LDLRAD2, RPS6KC1, LRP2, LZTR1, ALOX12, NFXL1, NPHS1, KIAA0040, MRAP, MT-ND4, MFSD1, NXPE2, PI4KA, IL23R, OR10R2, MRC1, GRIN2D, NDUFB9, NSMF, ABCC2, LRRC24, KCTD8, DYSF, DPP4, GPR63, LRRK2, NDST2, NPC1L1,
   g. Tissue protected: Heart (cardiomyocytes) TTYH3, TMED6, TCTN2, SLC30A4, SCN4B, SPAG4, TMEM138, SLC7A6, UCP3, DST, EFNB3, APLNR, CACNB2, GABARAPL2, RASL10B, SLC14A1, TMEM106B, SLC22A15, SLC47A1, MAPT, SYT13, PCSK5, SLC25A17, TGFBR3, SLC22A16, RAB17, SNX24, SUSD3, PITPNM1, RNF144A, SLC4A10, SPIRE1, SIRPB1, SGCB, NEGR1, BVES, PAG1, TPCN1, PTPRD, IL10RB, P2RY6, RECK, SLC25A20, PLN, PVR, RNF144B, GPIHBP1, MRGPRF, SEL1L3, SHISA4, INPP5D, FLT1, SLC25A36, SPATA9, SGCG, KCNQ4, MAPKAP1, SLC9A3, PCDHB15, SNX7, MPC2, MXRA7, PPP1R3A, PRRT2, OSBPL7, LANCL2, ITPR2, MFSD11, CNEP1R1, DMPK, S LC25A15, OBSCN, RFTN2, TMEM132A, TMEM236, SLC7A7, UST, TREML1, ZP2, QTRT1, TTC7B, TMEM41A, RINT1, TMEM86A, SEMA6B, SIRT2, C2orf88, SPHK1, SCAMP5, RAB27A, PIGZ, SLC6A12, RYR1, RUFY3, SNX6, VTI1B, SLCO2B1, SLC25A23, SUSD1, SEMA5B, ST8SIA2, TEX261, ARHGAP21, SLC35G2, ST8SIA5, VASN, TCTN3, TRPC6, TMEM39B, FKRP, FCER1A, EVA1A, GABBR1, GREB1, MMP27, KCNMB3, CX3CR1, CLEC16A, CKMT2, DSP, CACNA2D1, CDH15, ADRA1A, DCBLD1, CADM4, ADGRF1, RASGRP2, CAVIN4, CLN6, CREB3L2, FGFRL1, HHATL, CALN1, CYB561A3, FCGR2B, FXYD6, CHRNA1, ADGRA3, CD248, FXYD7, GPAT2, FGD2, CD1E, HHAT, DEPDC5, MALL, CAMK2B, KCNJ16, LRRC37B, CDH2, FES, MBOAT7, CPTP, LEPROTL1, MAN1C1, ADGRL4, AP3M1, CDK14, ABHD16A, CXCR1, FCGRT, ANKRD13B, B3GNT4, DMD, AP1S2, CDC42SE1, CLN3, CNTNAP3, CSF3R, SLC25A31, ADCK5, ATP1A2, CMKLR1, BSN, CADPS, CAV3, CPT1B, CDH13, CD1D, AKAP6, ADRB1, ASIC5, ARL4C, ANO5, BBS1, ARHGEF4, AP4E1, ATP8B1, BCL2L13, BACE2, ATG9A, CACNA1C, PAM, IBTK, ABCC5, NFASC, MYZAP, LFNG, LHFPL2, MFAP3L, NCAM1, GPR182, LIMS2, NDUFS1, KCND1, KCNK5, GPR15, GK, IGFLR1, LDLRAD2, SLC2A8, IL18RAP, RPS6KC1, LZTR1, ALOX12, NFXL1, MRAP, MT-ND4, PI4KA, OR10R2, NDUFB9, LRRC24, MCEMP1, DES, DYSF, GPR63, LRRK2, NDST2,
   h. Tissue protected: Thyroid TMED6, TMEM158, TCTN2, SLC30A4, SCN4B, TMEM138, DST, APLNR, CACNB2, GABARAPL2, TMEM100, TMEM106B, TRIM13, UBE2J1, VPS33B, SLC22A15, SLC47A1, SYT13, PCSK5, SLC25A17, TGFBR3, TMEM251, PIM1, ALPL, S LC22A16, SLITRK4, RAB17, SNX24, ENPP1, PCDHB2, RNF144A, SCNN1D, SPIRE1, SIRPB1, ARHGAP5, NEGR1, BVES, TPO, PAG1, TPCN1, VHL, IL10RB, RHBDD2, SLC25A20, PVR, RNF144B, GPIHBP1, LIN7A, SEL1L3, INPP5D, KCNQ4, MAPKAP1, PCDHB15, SNX7, SLC11A1, MPC2, MXRA7, MGLL, OSBPL7, LANCL2, PSD3, ITPR2, MFSD11, CNEP1R1, NTRK2, SLC25A15, OBSCN, SLC7A7, TMEM260, UST, QTRT1, TMEM128, TTC7B, TMEM41A, ZFYVE9, RINT1, TMEM86A, RTP5, SPHK1, SLC26A4, RUFY3, VTI1B, SLCO2B1, SLC25A23, SUSD1, TEX261, SREBF1, ARHGAP21, REEP4, TCTN3, TMEM39B, C19orf18, ACVR1B, EFHD1, IYD, FCER1A, EVA1A, GABBR1, GPR37, GREB1, CX3CR1, SLC48A1, CLEC16A, FCER1G, DAB2, DCBLD1, FYN, CADM4, CLCNKB, RASGRP2, CLCNKA, CLN6, CREB3L2, FGFRL1, CALN1, DMTN, FCGR2B, FXYD6, ADGRA3, CD99, GLMP, GPAT2, FGD2, CD1E, HCN1, IPCEF1, DEPDC5, MALL, LPXN, CAMK2B, KCNJ16, CDH2, FES, MAGI1, ADGRL4, AP3M1, CDK14, CXCR1, ANKRD13B, CLTCL1, ELMO2, AAK1, ATP9A, AQP4, ENPEP, ADCK5, CMKLR1, CRHR1, TMEM63B, ACVRL1, ARL4C, BBS1, ARHGEF4, AP4E1, ADGRV1, ATP8B1, CACNA1C, INTS2, CAVIN2, IBTK, MPP2, NFASC, MYZAP, LFNG, LHFPL2, NCAM1, NDUFS1, GFRA2, KCNK5, GPR15, GK, IGFLR1, IL17RA, LZTR1, MRAP, MT-ND4, MFSD1, PI4KA, OR10R2, GRIN2D, NDUFB9, LRRC24, NCS1, GPR63, NDST2,
   i. Tissue protected: Pancreatic endocrine cells (Islets) SLC30A4, SCN4B, MAP1LC3A, CACNB2, ACER3, SCGN, UBE2J1, MAPT, SYT13, SLC25A17, TGFBR3, PIM1, ALPL, STX1A, RAB3A, PITPNM1, RNF144A, RTN1, SCG3, ARHGAP5, NEGR1, POMGNT2, PAG1, TPCN1, VAT1, VHL, IL10RB, SLC25A20, PVR, RAB3B, UCHL1, SLC25A36, STX5, KCNQ4, MAPKAP1, PCDHB15, MPC2, MXRA7, PTPRN, LANCL2, PTH2R, KIRREL3, MFSD11, CNEP1R1, OBSCN, NECAB2, SLC17A6, ATP6V1D, SV2A, TSPAN7, TTC7B, TNS2, PLAUR, ZFYVE9, SLC30A8, RINT1, TMEM86A, STK10, SCAMP5, RAB27A, SYP, PTPRN2, RUFY3, VTI1B, SLC38A5, SLCO2B1, TEX261, MAPK10, ARHGAP21, SLC35G2, OPRD1, TSPAN2, VASN, TMEM39B, EMC4, DLK1, GREB1, DGCR2, KCNMB3, CLEC16A, CLTRN, GNAO1, CREB3L2, FAM169A, FGFRL1, CASR, FXYD6, ADGRA3, CD99, FKBP11, CDH2, MAGI1, CDK14, CXCR1, ENO2, ELMO2, ATP9A, CLU, FXYD2, ADCK5, AMPH, ATP1A2, CADPS, CRHR1, CD1D,ASIC5, ACVRL1, ARL4C, BBS1, ARHGEF4, EPB41L3, ADGRV1, ATP8B1, ATG9A, PAM, BBS7, GAD2, ALOX5, IBTK, MPP2, ABCC5, NFASC, LFNG, GNAZ, GPR153, GFRA2, KCND1, KIAA2013, IGFLR1, FMO1, LDLRAD2, MRAP, MT-ND4, PI4KA, OR10R2, NDUFB9, LRRC24, DPP4,
   j. Tissue protected: Pancreatic exocrine cells TMED6, TCTN2, SCN4B, TMEM138, GABARAPL2, RASL10B, TBC1D3D, TMEM100, UBE2J1, SYT13, PCSK5, PIM1, ALPL, SLC22A16, RAB17, ENPP1, SLC38A9, STIMATE, SLC27A2, SLC4A10, SPIRE1, SIRPB1, TMEM97, SEC16B, POMGNT2, PAG1, VAT1, VHL, IL10RB, PCDHA4, SLC25A20, PVR, RNF144B, PSCA, KCNH8, MRGPRF, SEL1L3, SLC25A36, SPATA9, STX5, STYK1, SCUBE1, PCDHB15, SNX7, MPC2, MXRA7, MGLL, PTH2R, NEURL1, ITPR2, MFSD11, CNEP1R1, OBSCN, ATP6V1D, TMEM72, SLC3A1, VSIG10, UST, UNC13D, TMEM128, TTC7B, TMEM41A, PLAUR, TBC1D3E, ZFYVE9, RINT1, TMPRSS2, C2orf88, SPHK1, SYCN, SCAMP5, RAB27A, RUFY3, VTI1B, SLC38A5, SLCO2B1, SLC4A4, SLC25A23, SEC11C, SLC19A1, SUSD1, ARHGAP21, REEP4, TCTN3, TMEM39B, ACVR1B, EFHD1, GGT1, DPEP1, CUZD1, EVA1A, GP2, SLC2A12, KIRREL2, FA2H, SLC48A1, CLEC16A, FCER1G, CYB5A, DSP, DAB2, CYSTM1, ADGRF1, RASGRP2, CLN6, CREB3L2, EVI2A, FGFRL1, FCGR2B, FXYD6, CD248, GPAT2, FKBP11, FGD2, CA4, HHAT, MALL, KCNJ16, LRRC37B, FES, MAGI1, LEPROTL1, MAN1C1, AP3M1, CDK14, AQP1, CDH9, AP1S2, CDC42SE1, CLN3, ACE2, ANPEP, CNTNAP3, CFTR, CLDN10, SLC25A31, FXYD2, ADCK5, CMKLR1, BSN, CADPS, CHST4, ARL4C, AP4E1, ADGRV1, ATP8B1, ATG9A, PIK3AP1, ALG14, BBS7, AQP12A, AQP12B, CAVIN2, IBTK, MPP2, LRFN5, MYZAP, LFNG, NCAM1, NDUFS1, GFRA2, KIAA2013, IGFLR1, IL17RA, RPS6KC1, NFXL1, MRAP, MT-ND4, OR10R2, NDUFB9, LRRK2, NDST2,
10. Cancer treated: skin cancer
   a. Tissue protected: Colon TSPAN12, AXL, TBC1D9B, TSPAN3, SCN4B, SFT2D2, SNX4, SMAGP, TMEM74B, SLC7A6, TNFSF14, MAP1LC3A, ATP8B2, C2CD2, CORO7, ACY3, FCAMR, G ABARAPL2, MGAT4C, SLC46A1, SCGN, SLC14A1, TMEM106B, TM4SF18, UBE2J1, SLC47A1, SYT13, PIM1, PLPP2, SELENBP1, STX1A, TREM2, RAB17, SEZ6L2, SNX24, TMEM263, ENPP1, SMIM24, SUSD3, TMEM94, TNFRSF11A, NAAA, PITPNM1, SLC27A2, RHOA, SOCS7, SMIM1, PEX1, PLXNA1, PEMT, SLC25A33, SLC25A46, NPDC1, NTRK1, ARHGAP5, RNF5, MIEF2, PEX11G, HCRTR1, SRD5A3, NAGPA, NBEA, BVES, P2RX6, PAG1, PIGR, PLEKHA3, RGS16, MUC12, TPCN1, TVP23B, TSPAN33, TULP3, TFPI, TJP3, WSCD1, PTPRD, RAB27B, KLRG1, RAB20, DLC1, RASD2, PCDHA4, LMAN2, PIGU, RNF139, PSCA, MRGPRF, SERP1, SPCS3, TPSG1, TRPM7, UPK3BL2, SLC18A1, SLC25A36, SPATA9, SLCO4A1, SPPL3, SLC9A3, IFI27, ITLN1, PTGDR, ADCYAP1R1, SYNGR4, TAOK2, MITD1, MPC2, PEX10, MRGPRX2, CD46, ABCB1, MGAT1, PCDH11X, KCNK1, MYORG, POMT2, HEG1, ILDR1, LYPLA1, KCNQ1, MGAT4A, MFSD11, NAALADL2, CNEP1R1, DENND5B, OBSCN, TMEM159, LDLRAD3, AATK, MEGF8, PROM1, NUCB2, PRIMA1, UGT2B4, SERP2, TMEM72, TNFAIP8L3, SELENOT, VPS11, SLC3A1, SIGIRR, TMEM132A, VSIG10, SYT11, VPS28, SLC7A7, TVP23C, VAMP1, TM4SF20, TMEM143, UTRN, VPS35L, ZP2, VAC14, QTRT1, SYTL4, TMEM64, TBCD, TSPAN31, TMEM41A, UNC13B, PLAUR, ZFYVE9, SYTL2, TMEM53, UPK3BL1, SLC46A2, TSC2, SLC35A3, MCUR1, SEZ6, OCLN, RNF125, SPHK1, SLC28A3, SCAMP5, RAB27A, RNF103, SYP, SUSD6, PIGZ, PTPRN2, SLC6A12, RUFY3 , SLC6A15, SERINC1, SLC19A2, TGFBR1, VTI1B, SIGLEC14, TMEM204, MTG2, PPM1L, SLC25A23, NAPG, SRPRB, SEC11C, SUSD1, TECPR1, PRRT3, IMMT, SLC35E1, SLC35B1, ARHGAP27, NAPB, RNF186, TMEM154, TMIGD2, VASN, TCTN3, TRPC6, VPS41, UBL3, TMEM39B, ADCK2, ATG2B, CDHR5, ARL4D, ACVR1B, GGT1, DPEP1, HTR7, FKRP, FMO5, IL9R, GHR, GREB1, FKTN, SLC2A12, KCNMB3, KIFC3, FPR1, SLC48A1, FCER1G, GOLPH3, CYP2S1, DMXL2, ABCG2, ACSL5, CLCA1, FAM171A1, GLP2R, CEACAM1, GCNT3, B4GALT4, B3GNT5, CACNA1B, FPR2, DOK7, FKBP2, CHDH, ENTPD7, GNB5, ADGRF1, HTR1A, CLN6, EMC10, DGKE, GATM, FZD2, HLA-DQB1, FXYD6, AQP8, CLMN, CNTFR, COQ2, DIAPH1, ADGRE1, CDIPT, ELOVL1, WDPCP, CHODL, CA12, CA4, CD1E, C6orf89, CERS4, HHAT, DEPDC5, HRH1, GNG11, DGKQ, LRRC37B, CTDNEP1, CPTP, DGAT1, HSD17B2, LIME1, MAGI1, ENTPD8, FPGS, LPP, MALRD1, MAN1C1, AP3M1, CDK14, ABHD16A, CXCR1, FER1L6, ANO8, BEST2, ENTPD1, BRI3, CYP26B1, CDC42EP5, MPP1, GAL3ST2, HTR4, B3GNT8, KDELR3, FIBP, ELOVL7, ERBIN, APOBR, CHPF2, CDHR2, SLC25A13, CRB3, ACE2, ANPEP, ATP9A, CNTNAP3, CDH17, CD300LF, CPM, SLC31A1, ATL2, CLCA4, CYB561, ALG2, SLC25A31, CLDN11, KCNIP3, ATP1A2, CADPS, ACKR3, COG8, ANKRD46, GRAMD1A, ARRDC1, ATRN, ADGRG6, ASIC5, ADGRL1, B3GNT6, CEACAM6, ACVRL1, BBS5, ACKR2, ARL4C, ANO1, ANXA13, BBS1, CHST5, ARHGEF4, ASPHD1, EPB41L3, ABHD12, AGPS, BCL2L13, CDH26, C12orf66, ATG9A, BLNK, ALG14, CASD1, CHMP2B, CEACAM3, DAPP1, B3GAT1, CASQ1, UGT8, DIP2A, FGFR4, IBTK, ABCC5, SMPD4, HMOX1, SLC9A3R2, PDZD3, NCEH1, KCNA5, LRFN5, MYZAP, LFNG, LHFPL2, MS4A18, GIPC1, NDUFS1, MC4R, MS4A12, GLCE, GFRA2, MCUB, SLC16A9, MSTO1, GNG12, GLRA2, SLC2A6, KIAA2013, KCNK10, NIPAL1, NIPAL4, SLC16A10, IGFLR1, LY9, LYN, GPA33, DPY19L1, LZTR1, ALOX12, MUC13 , NOD2, HPS6, MYOC, ICA1, NEU4, OR10R2, SLC16A7, MRC1, SLC16A4, NDUFB9, KLRC3, ITSN1, PRKCA, PDLIM4, GPR63, PSKH1, NDST2,
   b. Tissue protected: Lung TSPAN12, TGM2, AXL, VSIG4, SLC30A4, SFT2D2, SMAGP, TMEM74B, SLC7A6, EFNB3, LRP1, CD300C, CHRM3, SLC14A1, TRIM13, UBE2J1, SLC22A15, TGFBR3, SELENBP1, TREM2, RAB17, SUSD3, RNF144A, RHOA, PLXNA1, RNF5, MIEF2, BVES, AGER, PAG1, PIGR, PLLP, TVP23B, TSPAN33, TFPI, TJP3, SLC18A2, RASD2, REEP2, PTPRG, SPCS3, MGAT4B, SLC25A36, STX5, MPC2, MRGPRX2, CD46, KCNK1, POMT2, KCNQ1, MFSD11, EHD2, OBSCN, CD84, VPS11, SIGIRR, TMEM80, TVP23C, UTRN, QTRT1, PTPRB, TMEM255B, SEZ6, OCLN, RNF125, RHOG, RNF103, SUSD6, RUFY3, SLC6A15, SLC19A2, VTI1B, STEAP1, NAPG, SRPRB, TECPR1, VPS41, UBL3, ATG2B, ACVR1B, EHD1, FKTN, EHD4, FPR1, SLC48A1, CD247, FCER1G, CCR7, CYB5A, SLC44A2, DMXL2, ACSL5, FAM171A1, AOC3, ECEL1, FKBP2, CLDN18, GNB5, DGKE, EPHX1, HCLS1, HLA-DQB1, COQ2, CDIPT, ELOVL1, CERS4, CCDC88A, AIFM2, FES, LIME1, AP3M1, CAPN2, CXCR1, ABCA8, APBB1IP, CHPF2, ANK2, AQP4, ATL2, CYB561, CACNA2D2, ACE, BSN, ADGRL1, CEACAM6, ASPHD1, CACNA1C, CADM1, DAPP1, CXCR3, CAVIN2, IBTK, NCEH1, LAMP3, LFNG, MC4R, EHD3, MSTO1, GK, LDLRAD2, GALNT13, HPS6, PI4KA, SLC16A7, SLC16A4, ITSN1, SLC34A2, PSKH1, LRRK2,
   c. Tissue protected: Skin SCN4B, SFT2D2, SMAGP, SUN3, TMEM74B, C2CD2, CD300C, CHRM3, GABARAPL2, MGAT4C, SLC47A1, TGFBR3, PLPP2, RAB17, TMEM263, ENPP1, SLC6A1, PITPNM1, PLXNA1, TMEM97, NPDC1, ARHGAP5, RNF5, MIEF2, SRD5A3, NEGR1, SLC26A9, RGS16, TULP3, TFPI, RAB27B, PIGU, REEP2, RNF139, SERP1, SPCS3, MGAT4B, TYROBP, STX5, PCDH19, SLC9A3, IFI27, TAOK2, MITD1, MGAT1, KCNK1, HEG1, NAALADL2, DMPK, OBSCN, AATK, SERP2, SELENOT, VPS11, VPS28, SLC7A7, YRDC, TMEM260, VAMP1, UTRN, ZP2, VAC14, TBCD, TMEM255B, TSC2, SLC35A3, OCLN, RNF125, SPHK1, SLC28A3, S1PR5, SUSD6, SLC19A2, VTI1B, TMEM204, SLC19A1, PRRT3, NAPB, TRPC6, VPS41, TMEM39B, ADCK2, CYBA, FFAR2, FA2H, FPR1, CYP2W1, DSP, DMXL2, GLP2R, ANO7, CACNA2D1, FPR2, FKBP2, GATM, ATRAID, FXYD6, CD99, CD248, COX14, ELOVL1, WDPCP, CA12, C6orf89, CERS4, HCN1, DGKQ, LRRC37B, CTDNEP1, EDNRB, DGAT1, LPP, MALRD1, AP3M1, CDK14, B3GNT4, ABCA8, BRI3, KDELR3, FIBP, ELOVL7, APOBR, CD81, SLC25A13, ANK2, CYB561, SLC25A31, MS4A1, CAV3, COG8, CD1D, ASPRV1, ASIC5, ADGRL1, TMEM63B, BBS5, ACKR2, ASPHD1, AGPS, BCL2L13, ATP12A, BLNK, DAPP1, IBTK, ABCC1, ABCC5, MYZAP, LFNG, GIPC1, MC4R, MCUB, SLC16A9, M STO1, GNG12, NIPAL1, NIPAL4, LDLRAD2, ALOX12, NOD2, PI4KA, OR10R2, SLC16A4, NDUFB9, NCS1, PSKH1, TSPAN12, TBC1D9B, TCTN2, SLC30A4, SCN4B, SNX4, CORO7, TRIM13, UBE2J1, SYT13, SUSD3, TNFRSF11A, TYR, PEMT, SLC25A33, NTRK1, NAGPA, PDGFC, PLLP, TPCN1, TSPAN33, TJP3, WIPI2, DLC1, MPP6, RASA4, SLCO4A1, SUSD4, KCNQ4, MPC2, CD46, MYORG, KCNQ1, DENND5B, SLC25A15, NUCB2, PRIMA1, SIGIRR, TMEM80, TMEM143, QTRT1, TMEM64, DCT, TSPAN31, UNC13B, PRNP, TMEM53, SNTB1, MCUR1, RHOG, RUFY3, SERINC1, RAB11FIP5, MTG2, NAPG, TECPR1, PMEL, ARHGAP27, TMIGD2, TCTN3, EHD1, GREB1, GSDMC, KCNMB3, KIFC3, EHD4, CYB5A, FAM171A1, B4GALT4, B3GNT5, CACNA1B, DOK7, EMC10, EVI2A, FZD2, CYB561A3, FCGR2B, CNTFR, GLMP, DIAPH1, ADGRE1, GPAT2, FGD2, HRH1, SLC2A5, CPTP, CXCR1, FNBP1, B3GNT8, ERBIN, CHPF2, ATP9A, SLC31A1, ATL2, ATRN, ANO1, BBS1, CPNE2, AP4E1, ATG2A, CACNA1C, ALG14, CHMP2B, CAVIN2, HAS3, NCEH1, KCNA5, LHFPL2, MUC21, NDUFS1, GLCE, GFRA2, EHD3, KIT, LZTR1, PDLIM4, GPR63,
   d. Tissue protected: Liver TSPAN12, VNN1, AXL, TBC1D9B, VDAC3, SCN4B, SFT2D2, SNX4, SMAGP, TNFSF14, UPK1B, LRP1, MAP1LC3A, C2CD2, CD300C, CREB3L3, CHRM3, ACY3, FCAMR, GABARAPL2, SLC14A1, TRIM13, TM4SF18, UBE2J1, SLC47A1, SYT13, PIM1, PLPP2, SELENBP1, TREM2, RAB17, SLC6A4, SNX24, ENPP1, SYNE3, SUSD3, TMEM94, NAAA, RNF144A, SLC25A40, SLC27A2, SLCO1B3, SLCO1B1, PLXNA1, PEMT, SLC25A33, SLC25A46, SLC25A43, SLC27A5, NPDC1, NTRK1, RNF5, PEX11G, SRD5A3, NAGPA, NEGR1, PAG1, PLEKHA3, TPCN1, TFPI, TJP3, SLC18A2, PTPRD, RAB27B, KSR2, RAB20, PTGDR2, LMAN2, PIGU, LIN7A, SERP1, SPCS3, MGAT4B, RASA4, SLC25A36, STX5, SPPL3, KCNQ4, PCDH19, SYNGR4, TAOK2, MITD1, MPC2, PRRT2, MRGPRX2, CD46, ABCB1, MGAT1, KCNK1, NAT8, MYORG, HEG1, MAGI2, ILDR1, LYPLA1, KCNQ1, MFSD11, DENND5B, SLC25A15, OBSCN, TMEM159, LDLRAD3, AATK, MEGF8, NUCB2, UGT2B4, SERP2, SELENOT, SLC3A1, SIGIRR, TMEM132A, YIPF1, TMEM260, VAMP1, UTRN, VPS35L, SYTL4, TMEM64, TNS2, TBCD, TSPAN31, UNC13B, ZDHHC13, PTPRB, TMEM53, TSC2, ST7L, OCLN, SPHK1, SUSD6, SLC6A12, SNX19, RUFY3, SERINC1, SLC19A2, TGFBR1, VTI1B, SLCO2B1, TMEM204, PPM1L, NAPG, SRPRB, TECPR1, TEX261, IMMT, SLC35E1, NAPB, TSPAN2, VASN, VPS41, TM4SF4, CDHR5, ACVR1B, GGT1, FKRP, FMO5, EVA1A, GHR, GPR37, GREB1, FKTN, KCNMB3, KIFC3, SLC48A1, CYP2W1, CYP2D6, CYB5A, CYP2S1, DMXL2, GJA5, ACSL5, FAM171A1, CACNA2D1, CEACAM1, B4GALT4, B3GNT5, CACNA1B, CACNA1A, FPR2, GAS2, DOK7, SLC2A2, CHDH, ENTPD7, HTR1A, CREB3L2, EMC10, CYP2A7, GATM, EPHX1, FZD2, FOLH1, CYP1A2, FXYD6, CNTFR, COQ2, COX14, CYP3A4, DIAPH1, CDIPT, CYP2A6, ELOVL1, CYP2B6, CD1E, CERS4, FMO3, HHAT, CYP4V2, DEPDC5, KCNJ16, NINJ1, CYP2C8, DHCR24, LRRC37B, CDH2, CTDNEP1, FES, KMO, DGAT1, HSD17B2, LIME1, MAL2, ENTPD8, MALRD1, MAN1C1, AP3M1, CDK14, CYP2C19, BCL2L10, AQP9, ENTPD1, ABCA8, BRI3, CYP26B1, FIBP, ERBIN, CHPF2, CDHR2, SLC25A13, CRB3, ANPEP, ASGR1, CPM, SLC31A1 , ADRA2B, ABCB11, ATP1A2, CMKLR1, CYP2A13, ACKR3, COG8, CYP2E1, GRAMD1A, ARRDC1, ATRN, ASIC5, ADGRL1, TMEM63B, ANO1, BBS1, ASPHD1, EPB41L5, AGPS, BACE2, CD320, ASGR2, ATG2A, CYP3A43, CHMP2B, DAPP1, B3GAT1, CASQ1, UGT8, INTS2, CAVIN2, FGFR4, IBTK, SMPD4, MOXD1, SLC9A3R2, MYZAP, LFNG, LHFPL2, MFAP3L, GIPC1, LIMS2, NDUFS1, GLCE, KCND1, SLC16A9, MSTO1, GPR15, GK, SLC2A6, NIPAL1, NIPAL4, IGFLR1, LDLRAD2, DPY19L1, SLC2A8, LZTR1, ALOX12, HPS6, PI4KA, ICA1, OR10R2, SLC16A4, NDUFB9, ITSN1, ABCC2, PDLIM4, DPP4, GPR63, PSKH1, LRRK2, NDST2, SLC10A1, NPC1L1,
   e. Tissue protected: Kidney (glomeruli) AXL, SCN4B, SFT2D2, SNX4, CORO7, MGAT4C, SLC14A1, TM4SF18, UBE2J1, SLC22A15, SLC47A1, SYT13, TGFBR3, TREM2, RAB17, SYNE3, SUSD3, TMEM94, SLC45A1, RNF144A, RHOA, PLXNA1, PEMT, PODXL, KIRREL1, PDGFC, P2RX6, PDGFRB, PLEKHA3, TVP23B, TSPAN33, TFPI, KSR2, RNF139, PTPRG, MRGPRF, SERP1, SPCS3, SLC34A1, TPSG1, SLC25A36, STX5, PTGDR, ADCYAP1R1, MPC2, PLA2R1, CD46, MGAT1, PTH2R, KCNK1, POMT2, MAGI2, MAST1, KCNQ1, MGAT4A, MAP6, NOS3, DENND5B, EHD2, MPP5, NKD1, LDLRAD3, NPHS2, SERP2, VPS11, SIGIRR, VPS28, TVP23C, UTRN, VPS35L, VAC14, TSPAN7, TTC7B, TNS2, ZFYVE9, TMEM255B, TMEM53, STK10, RNF125, SNCA, RNF103, SUSD6, SLC6A12, SLC19A2, VTI1B, THSD7A, NAPG, PTPRO, SLC35B1, NAPB, VASN, VPS41, ARL4D, HTR7, EHD1, EVA1A, HYAL2, EHD4, DMXL2, FAM171A1, CEACAM1, CACNA1B, GAS2, CD93, FAM107A, GNB5, ADGRF1, CD34, HTR1A, EMC10, EVI2A, FZD2, HLA-DQB1, ENG, CD99, CD248, DOCK8, ADGRE1, CR1, FGD2, HRH1, MAS1, LIME1, LPP, ADGRL4, CAPN2, ANO8, FNBP1, CRB3, ACKR1, ATL2, CLCA4, CHRM1, BSN, ATRN, ASPHD1, EPB41L5, AKAP12, CACNA1C, DAPP1, B3GAT1, ABCC1, SLC9A3R2, HAS3, KCNA5, GIPC1, LIMS2, ITGA8, SLC16A9, EHD3, MSTO1, GNG12, MLIP, NIPAL4, LDLRAD2, LZTR1, NPHS1, HPS6, PI4KA, NEU4, SLC16A7, SLC16A4, DYSF, DPP4, PSKH1, PDGFRA,
   f. Tissue protected: Kidney (tubules) TMEM231, TSPAN12, VNN1, AXL, TBC1D9B, TSPAN3, VDAC3, SCN4B, SFT2D2, SNX4, SMAGP, SUN3, TRPM3, TBC1D3G, TMEM74B, TREH, TNFSF14, ATP8B2, C2CD2, CORO7, CHRM3, ACY3, FCAMR, GABARAPL2, MGAT4C, THY1, SLC14A1, TM4SF18, UBE2J1, SLC22A15, SLC47A1, SELPLG, PIM1, PLPP2, SELENBP1, TREM2, RAB17, SLC6A4, SEZ6L2, TMEM263, ENPP1, SMIM24, SYNE3, SLC5A12, SLC5A2, SUSD3, TMEM94, STIMATE, SLC12A1, NAAA, PCDHB2, PITPNM1, RNF144A, SLC25A40, SLC27A2, SLC4A10, SCNN1D, RHOA, SMIM1, PEX1, PLXNA1, PEMT, SLC6A19, SLC22A11, S1PR2, SLC34A3, SLC25A33, SLC25A46, NAV3, NTRK1, ARHGAP5, RNF5, MIEF2, PEX11G, SLC28A2, HCRTR1, SRD5A3, RDH11, NAGPA, NEGR1, PTH1R, KIRREL1, PDGFC, NBEA, P2RX6, AGER, PIGR, RGS16, PLLP, TPCN1, TVP23B, TULP3, TFPI, TJP3, WSCD1, WIPI2, VHL, PTPRD, RAB27B, RAB20, DLC1, RASD2, MPP6, PCDHA4, PDE2A, LMAN2, PIGU, RALB, RNF139, PSCA, LIN7A, MRGPRF, UCHL1, SERP1, SPCS3, MGAT4B, SLC34A1, TRPM7, UPK3BL2, RASA4, SLC25A36, STX5, PRICKLE1, SLCO4A1, SPPL3, SUSD4, KCNQ4, PCDH19, SLC9A3, IFI27, PTGDR, ADCYAP1R1, MITD1, NOS1, MPC2, PEX10, PRRT2, CD46, ABCB1, MGAT1, KCNK1, NAT8, MYORG, PANX2, POMT2, HEG1, MAGI2, ILDR1, LYPLA1, KCNQ1, MGAT4A, ITPR1, MFSD11, NAALADL2, CNEP1R1, NTRK2, DENND5B, EHD2, MPP5, SLC25A15, OBSCN, TMEM159, LDLRAD3, AATK, MEGF8, PROM1, PRIMA1, UGT2B4, ATP6V1B1, SLC22A6, SERP2, TMEM72, TNFAIP8L3, SELENOT, VPS11, SLC3A1, SIGIRR, TMEM132A, VSIG10, YIPF1, VPS28, ATP6V0A4, SLC7A7, TMEM80, TMEM260, TVP23C, VAMP1, UTRN, VPS35L, ZP2, VAC14, QTRT1, SYTL4, TMEM252, TMEM174, TMEM64, TTC7B, TBCD, TSPAN31, TMEM41A, UNC13B, PLAUR, ZFYV E9, ZDHHC13, PTPRB, SYTL2, TMPRSS2, TMEM255B, TMEM53, FASLG, UPK3BL1, SLC46A2, TSC2, SLC28A1, SEMA6B, SNTB1, SIRT2, MCUR1, SLC22A2, OCLN, RNF125, SPHK1, RHOG, SLC28A3, SCAMP5, RNF103, SLC22A8, SLC13A3, SUSD6, PIGZ, SLC6A12, SNX19, RUFY3, RAB11FIP5, SLC19A2, SLC6A18, SLC13A2, TGFBR1, TMEM213, VTI1B, SLC5A1, SIGLEC14, SYT7, TMEM204, MTG2, TMEM184A, SLC4A4, SLC25A23, NAPG, SRPRB, SEC11C, TECPR1, TEX261, PKHD1, PRRT3, IMMT, SLC35E1, SLC35G2, OPRD1, NAPB, TRPV5, TSPAN2, TMIGD2, VASN, VPS41, UBL3, TMEM39B, ADCK2, ATG2B, CDHR5, ARL4D, ENPP6, EMC4, ACVR1B, GGT1, IYD, DPEP1, ENPP3, CUBN, CLSTN2, EHD1, FKRP, FMO5, EVA1A, GHR, GREB1, HYAL2, FKTN, GSDMC, KCNMB3, KIFC3, KIRREL2, EHD4, SLC48A1, FCER1G, CKMT2, CPNE6, CYB5A, JAKMIP1, CLTRN, CYP2S1, SLC44A2, DMXL2, GJA5, ACSL5, FAM171A1, B4GALT4, B3GNT5, CACNA1B, GAS2, DOK7, FAM107A, FNDC5, FKBP2, SLC2A2, CHDH, DOCK2, GNB5, CLCNKB, HTR1A, ADGRG5, CALCR, CLCNKA, CLN6, CREB3L2, EMC10, EVI2A, FAM169A, GATM, FZD2, ATRAID, CASR, FOLH1, HLA-DQB1, FXYD6, CLMN, CNTFR, CD248, COQ2, COX14, DIAPH1, ADGRE1, CDIPT, ELOVL1, WDPCP, FRRS1L, CYP2B6, CA12, CA4, CD1E, C6orf89, CERS4, FMO3, HHAT, CYP4V2, DEPDC5, HRH1, GNG11, GLIPR1L2, LPXN, DGKQ, KIAA0319, KCNJ16, DHCR24, LRRC37B, CDH2, CTDNEP1, FES, KMO, EDNRB, SLC2A5, LRFN4, CPTP, DGAT1, LIME1, MAL2, MAGI1, FPGS, LPP, MALRD1, MAN1C1, AP3M1, CDK14, ABHD16A, BSND, BCL2L10, CAPN2, CXCR1, ANO8, AQP6, ABCA8, BRI3, CYP26B1, CDC42EP5, NPR3, MPP1, HTR4, FIBP, ERBIN, CDC42SE1, CHPF2, CDHR2, SLC25A13, CRB3, ADAM28, ANK2, ANPEP, ATP9A, SLC7A9, CPM, CLDN10, DUSP15, SLC31A1, ATL2, CLCA4, ALG2, SLC25A31, FXYD2, ACE, CATIP, CMKLR1, BSN, ACKR3, CLDN2, CAV3, COG8, CPT1B, COX7B, ANKRD46, GRAMD1A, ARRDC1, ATRN, ASIC5, ADGRL1, TMEM63B, ACVRL1, BBS5, ACKR2, ARL4C, ANO1, ANXA1 3, BBS1, CPNE2, ARHGEF4, AP4E1, ASPHD1, EPB41L3, EPB41L5, AGPS, C12orf66, ATP12A, ATG9A, BLNK, C5AR1, CACNA1C, CYP3A43, CDH16, CHMP2B, CADM1, B3GAT1, CASQ1, UGT8, INTS2, DIP2A, FGFR4, IBTK, ABCC5, NLGN1, SMPD4, HMOX1, LRFN2, MOXD1, PDZD3, HAS3, NCEH1, KCNA5, MYZAP, LFNG, LHFPL2, KCNJ10, KCNJ12, MFAP3L, GIPC1, NDUFS1, MC4R, GLCE, GFRA2, KCND1, KCNK5, MCUB, SLC16A9, EHD3, MSTO1, GNG12, GK, MS4A4A, MLIP, SLC2A6, KIAA2013, KCNK10, NIPAL1, NIPAL4, SLC16A10, IGFLR1, FAM234B, FMO1, LDLRAD2, DPY19L1, LRP2, LZTR1, ALOX12, NPHS1, HPS6, MYOC, MFSD1, PI4KA, ICA1, IL23R, OR10R2, SLC16A7, MRC1, SLC16A4, GRIN2D, NDUFB9, ITSN1, ABCC2, LRRC24, MDGA2, KCTD8, PRKCA, PDLIM4, DYSF, DPP4, GPR63, PSKH1, LRRK2, NDST2, NPC1L1,
   g. Tissue protected: Heart (cardiomyocytes) TSPAN12, TGM2, AXL, TBC1D9B, TCTN2, TSPAN3, VDAC3, SLC30A4, SLC27A6, SCN4B, SMAGP, SPAG4, TMEM74B, SLC7A6, TNFSF14, UCP3, EFNB3, ATP8B2, CAP2, CD300C, GABARAPL2, MGAT4C, SLC46A1, SLC14A1, TMEM106B, SLC22A15, SLC47A1, SYT13, TGFBR3, PLPP2, TREM2, RAB17, SEZ6L2, SNX24, SUSD3, TMEM94, NAAA, PITPNM1, RNF144A, SLC4A10, RHOA, PEX1, PLXNA1, SGCB, PEMT, SLC25A33, NPDC1, NTRK1, MIEF2, PEX11G, SRD5A3, NAGPA, NEGR1, BVES, P2RX6, PAG1, PLEKHA3, TPCN1, TVP23B, TSPAN33, TFPI, TJP3, WSCD1, PTPRD, RAB20, POPDC2, MPP6, P2RY6, MRGPRF, SERP1, SPCS3, SHISA4, INPP5D, MGAT4B, FLT1, RASA4, SLC25A36, SPATA9, SGCG, SLCO4A1, SRL, KCNQ4, PCDH19, SLC9A3, MITD1, MPC2, PRRT2, CD46, MGAT1, KCNK1, ART3, MYORG, POMT2, MAGI2, ILDR1, LYPLA1, KCNQ1, MGAT4A, ITPR1, MFSD11, CNEP1R1, DENND5B, DMPK, EHD2, SLC25A15, OBSCN, TMEM159, AATK, MEGF8, NUCB2, PRIMA1, SERP2, SIPA1, RYR2, SELENOT, VPS11, SIGIRR, TMEM132A, YIPF1, SLC7A7, YRDC, TVP23C, TMEM143, TREML1, UTRN, VPS35L, ZP2, VAC14, QTRT1, TMEM64, TTC7B, TBCD, TSPAN31, TMEM41A, ZDHHC13, PTPRB, TSC2, SEMA6B, SNTB1, SIRT2, SPHK1, SCAMP5, RAB27A, RAB11FIP4, SUSD6, PIGZ, SLC6A12, SNX19, SLAMF8, RUFY3, SERINC1, SLC19A2, TGFBR1, VTI1B, SLCO2B1, TMEM204, MTG2, PPM1L, SLC25A23, NAPG, SRPRB, SUSD1, TECPR1, TEX261, IMMT, SLC35B1, SLC35G2, NAPB, ST8SIA5, VASN, TCTN3, TRPC6, VPS41, UBL3, TMEM39B, ATG2B, EHD1, FKRP, EVA1A, GREB1, MMP27, FKTN, KCNMB3, EHD4, FPR1, CKMT2, CYP2W1, GOLPH3, CYP2S1, DSP, DMXL2, ABCG2, FAM171A1, CACNA2D1, CDH15, ADRA1A, B4GALT4, B3GNT5, CACNA1B, DOK7, FNDC5, CHDH, GNB5, ADGRF1, HTR1A, CAVIN4, CLN6, CREB3L2, EMC10, SLC2A4, DGKE, HHATL, FZD2, CYB561A3, HLA-DQB1, FCGR2B, FXYD6, CHRNA1, CNTFR, CD248, CDIPT, ELOVL1, WDPCP, GPAT2, FGD2, CD1E, C6orf89, CERS4, HHAT, CYP4V2, DEPDC5, HRH1, DGKQ, CAMK2B, KCNJ16, LRRC37B, CDH2, CTDNEP1, FES, MBOAT7, CPTP, DGAT1, LIME1, LEPROTL1, LPP, MALRD1, MAN1C1, ADGRL4, AP3M1, CDK14, ABHD16A, CXCR1, FCGRT, ANO8, B3GNT4, ENTPD1, B3GNT8, FIBP, DMD, ELOVL7, ERBIN, CDC42SE1, CD81, CHPF2, CDHR2, SLC25A13, ADAM28, ANK2, CNTNAP3, SLC31A1, CSF3R, ATL2, CL CA4, CYB561, ALG2, SLC25A31, ATP1A2, CMKLR1, BSN, CADPS, ACKR3, CCR4, CAV3, COG8, CPT1B, COX7B, ANKRD46, CD1D, ARRDC1, AKAP6, ADGRG6, ADRB1, ASIC5, BBS5, ARL4C, ANO1, BBS1, CPNE2, ARHGEF4, AP4E1, ASPHD1, BCL2L13, BACE2, ATG9A, BLNK, CACNA1C, DAPP1, CASQ1, UGT8, IBTK, JPH1, ABCC5, NLGN1, SMPD4, NCEH1, KCNA5, MYZAP, LFNG, LHFPL2, MFAP3L, NCAM1, GIPC1, GPR182, LIMS2, NDUFS1, MC4R, GLCE, KCND1, KCNK5, MCUB, SLC16A9, EHD3, MSTO1, GPR15, GK, MLIP, NIPAL4, SLC16A10, IGFLR1, LDLRAD2, DPY19L1, SLC2A8, IL18RAP, LZTR1, ALOX12, NOD2, PI4KA, ICA1, OR10R2, SLC16A4, NDUFB9, ITSN1, LRRC24, MCEMP1, PRKCA, PDLIM4, DES, DYSF, GPR63, PSKH1, LRRK2, NDST2,
   h. Tissue protected: Thyroid TSPAN12, TBC1D9B, TCTN2, TSPAN3, SLC30A4, SLC27A6, SCN4B, SFT2D2, SNX4, SMAGP, TMEM74B, TNFSF14, ATP8B2, C2CD2, CHRM3, GABARAPL2, SLC46A1, TMEM100, TMEM106B, TRIM13, TM4SF18, UBE2J1, VPS33B, SLC22A15, SLC47A1, SYT13, TGFBR3, TMEM251, PIM1, PLPP2, SELENBP1, SLITRK4, TREM2, RAB17, SNX24, TMEM263, ENPP1, TMEM94, TNFRSF11A, NAAA, PCDHB2, RNF144A, SCNN1D, RHOA, SMIM1, PEX1, PLXNA1, PEMT, SLC25A33, SLC25A46, NPDC1, NTRK1, ARHGAP5, RNF5, MIEF2, PEX11G, SRD5A3, NAGPA, NEGR1, KCNQ5, NBEA, BVES, TPO, PAG1, PLEKHA3, RGS16, TPCN1, TVP23B, TSPAN33, TULP3, TFPI, TJP3, WSCD1, WIPI2, VHL, RAB20, RASD2, MPP6, LMAN2, PIGU, RNF139, LIN7A, SERP1, SPCS3, INPP5D, UPK3BL2, PRICKLE1, SLCO4A1, SUSD4, KCNQ4, PCDH19, IFI27, TAOK2, MITD1, MPC2, CD46, KCNK1, PSD3, POMT2, HEG1, MAGI2, ILDR1, LYPLA1, MAST1, KCNQ1, MGAT4A, MFSD11, CNEP1R1, NTRK2, DENND5B, SLC25A15, OBSCN, TMEM159, LDLRAD3, AATK, MEGF8, PROM1, NUCB2, PORCN, PRIMA1, SERP2, SELENOT, VPS11, SIGIRR, YIPF1, VPS28, SLC7A7, YRDC, TMEM80, TMEM260, TVP23C, UTRN, VPS35L, VAC14, QTRT1, SYTL4, TTC7B, TBCD, TSPAN31, TMEM41A, UNC13B, ZFYVE9, TMEM255B, TMEM53, FASLG, UPK3BL1, TNFRSF10C, TSC2, RTP5, MCUR1, OCLN, RNF125, SPHK1, RNF103, SLC26A4, SUSD6, SNX19, RUFY3, SERINC1, SLC19A2, VTI1B, RNFT2, SLCO2B1, TMEM204, MTG2, PPM1L, SLC25A23, NAPG, SRPRB, SUSD1, TECPR1, TEX261, PRRT3, IMMT, SLC35E1, TRPV5, TMIGD2, TCTN3, VPS41, UBL3, TMEM39B, ADCK2, C19orf18, ACVR1B, IYD, DUOX1, HTR7, EHD1, EVA1A, GHR, GPR37, GREB1, FKTN, KIFC3, EHD4, SLC48A1, FCER1G, CYP2W1, GOLPH3, CYP2S1, DMXL2, ABCG2, FAM171A1, B3GNT5, FPR2, DOK7, FKBP2, GNB5, CLCNKB, EPHX4, HTR1A, CLCNKA, CLN6, CREB3L2, EMC10, FCRL1, DGKE, GATM, FZD2, ATRAID, HLA-DQB1, FCGR2B, FXYD6, CD99, CLMN, CNTFR, GLMP, COQ2, COX14, DIAPH1, ADGRE1, CDIPT, ELOVL1, WDPCP, GPAT2, FGD2, CD1E, C6orf89, CERS4, HCN1, FMO3, IPCEF1, DEPDC5, LPXN, DGKQ, CAMK2B, KCNJ16, CDH2, CTDNEP1, FES, SLC2A5, DGAT1, LIME1, MAGI1, MALRD1, ADGRL4, AP3M1, CDK14, FZD5, CAPN2, CXCR1, FER1L6, ANO8, ENTPD1, FNBP1, FIBP, ERBIN, CHPF2, SLC25A13, CRB3, ANK2, AAK1, ATP9A, AQP4, CLCA4, CYB561, ALG2, CMKLR1, CRHR1, ACKR3, COG8, ANKRD46, ARRDC1, ATRN, ADGRL1, TMEM63B, ACVRL1, BBS5, ACKR2, ARL4C, BBS1, CPNE2, ARHGE F4, AP4E1, AKAP12, AGPS, CDH26, CD320, C12orf66, BLNK, C5AR1, CACNA1C, CDH16, CDH10, CHMP2B, CEACAM3, CADM1, ARFGAP3, UGT8, INTS2, AP3B2, CAVIN2, IBTK, NLGN1, SMPD4, MTNR1B, NCEH1, KCNA5, MYZAP, LFNG, LHFPL2, NCAM1, GIPC1, NDUFS1, MC4R, GLCE, GFRA2, KCNK5, SLC16A9, MSTO1, GNG12, GPR15, GK, SLC2A6, NIPAL4, IGFLR1, LYN, FAM234B, LZTR1, HPS6, MFSD1, PI4KA, ICA1, OR10R2, SLC16A4, GRIN2D, NDUFB9, LRRC24, NCS1, PRKCA, PDLIM4, GPR63, PSKH1, NDST2,
   i. Tissue protected: Pancreatic endocrine cells (Islets) TSPAN12, TBC1D9B, TSPAN3, VDAC3, SLC30A4, SCN4B, SFT2D2, SNX4, SMAGP, STXBP1, TNFSF14, MAP1LC3A, CHRM3, SCGN, UBE2J1, SYT13, TGFBR3, PIM1, PLPP2, STX1A, TREM2, SEZ6L2, NAAA, RAB3A, PITPNM1, RNF144A, RHOA, SMIM1, PLXNA1, RTN1, PEMT, SLC25A33, SLC25A46, NPDC1, NTRK1, SCG3, ARHGAP5, RNF5, MIEF2, HCRTR1, SRD5A3, NEGR1, NBEA, POMGNT2, PAG1, RGS16, TPCN1, TVP23B, TFPI, TJP3, WSCD1, WIPI2, VHL, RAB20, RASD2, MPP6, PTGDR2, RNF139, UCHL1, SERP1, SPCS3, UPK3BL2, SLC25A36, STX5, PRICKLE1, SPPL3, KCNQ4, TAOK2, MITD1, MPC2, PTPRN, CD46, PTH2R, KCNK1, MYORG, POMT2, HEG1, MAGI2, ILDR1, MGAT4A, MFSD11, CNEP1R1, OBSCN, TMEM159, AATK, NECAB2, NUCB2, SLC17A6, ATP6V1B1, SERP2, SHISAL2B, VPS11, SV2A, YRDC, TVP23C, VAMP1, TM4SF20, UTRN, VAC14, SYTL4, TSPAN7, TMEM64, TTC7B, TNS2, TSPAN31, UNC13B, PLAUR, ZFYVE9, ZDHHC13, SLC30A8, PRNP, TMEM255B, TMEM53, FASLG, UPK3BL1, TSC2, SLC35A3, STK10, RNF125, SCAMP5, RAB27A, RNF103, SYP, PTPRN2, SNX19, RUFY3, SLC19A2, VTI1B, SLCO2B1, SIGLEC14, SYT7, TMEM204, PPM1L, NAPG, SRPRB, TECPR1, TEX261, MAPK10, PRRT3, IMMT, SLC35G2, OPRD1, NAPB, TSPAN2, VASN, VPS41, TMEM39B, ATG2B, EMC4, HTR7, FFAR2, FMO5, GREB1, DGCR2, KCNMB3, CYP2W1, GOLPH3, CLTRN, DMXL2, FAM171A1, B4GALT4, B3GNT5, CACNA1B, DOK7, FKBP2, GNB5, GNAO1, CREB3L2, FAM169A, FBXO2, CASR, FXYD6, AQP8, CD99, CLMN, CNTFR, COQ2, DIAPH1, CDIPT, ELOVL1, WDPCP, CYP2B6, FKBP11, C6orf89, CERS4, CYP4V2, DGKQ, CDH2, CTDNEP1, MAGI1, MALRD1, CDK14, CAPN2, CXCR1, FIBP, CD81, CHPF2, SLC25A13, ENO2, ATP9A, CLU, ATL2, CYB561, ALG2, FXYD2, AMPH, ATP1A2, CADPS, CRHR1, ACKR3, COG8, ANKRD46, CD1D, ARRDC1, ATRN, ASIC5, ADGRL1, ACVRL1, BBS5, ARL4C, BBS1, ARHGEF4, EPB41L3, EPB41L5, CDH26, ATG9A, CADM1, GAD2, DAPP1, ARFGAP3, ALOX5, FGFR4, IBTK, ABCC5, NLGN1, SMPD4, LFNG, GNAZ, GIPC1, MC4R, GLCE, GFRA2, KCND1, MSTO1, KIAA2013, IGFLR1, FMO1, LDLRAD2, HPS6, PI4KA, ICA1, OR10R2, NDUFB9, LRRC24, MDGA2, PRKCA, PDLIM4, DPP4, PSKH1,
   j. Tissue protected: Pancreatic exocrine cells TSPAN12, AXL, TBC1D9B, TCTN2, VDAC3, SCN4B, SFT2D2, SMAGP, TNFSF14, ATP8B2, C2CD2, CD300C, CORO7, CHRM3, GABARAPL2, SLC46A1, TBC1D3D, TMEM100, TM4SF18, UBE2J1, SELPLG, SYT13, PIM1, PLPP2, TREM2, RAB17, SEZ6L2, TMEM263, ENPP1, TMEM94, STIMATE, NAAA, SLC27A2, SLC4A10, RHOA, SMIM1, PEX1, PLXNA1, PEMT, TMEM97, SLC25A33, SLC25A46, NTRK1, SEC16B, RNF5, MIEF2, SRD5A3, PDGFC, NBEA, POMGNT2, PAG1, PLEKHA3, RGS16, PLLP, TVP23B, TSPAN33, TFPI, TJP3, WSCD1, VHL, RAB27B, RASD2, MPP6, PCDHA4, LMAN2, PIGU, RNF139, PSCA, MRGPRF, SERP1, SPCS3, UPK3BL2, SLC25A36, SPATA9, STX5, SLCO4A1, SUSD4, IFI27, MITD1, MPC2, MRGPRX2, CD46, MGAT1, PTH2R, NEURL1, KCNK1, MYORG, POMT2, HEG1, LYPLA1, KCNQ1, MGAT4A, MFSD11, CNEP1R1, DENND5B, OBSCN, TMEM159, LDLRAD3, AATK, MEGF8, PROM1, NUCB2, PRIMA1, SERP2, TMEM72, TNFAIP8L3, SELENOT, VPS11, SLC3A1, SIGIRR, VSIG10, YRDC, TMEM80, TVP23C, VAMP1, UTRN, VAC14, SYTL4, UNC13D, TTC7B, TRAT1, TSPAN31, TMEM41A, UNC13B, PLAUR, TBC1D3E, ZFYVE9, ZDHHC13, PTPRB, SYTL2, TMPRSS2, TMEM255B, TMEM53, FASLG, UPK3BL1, TNFRSF10C, SNTB1, OCLN, RNF125, SPHK1, SYCN, SLC28A3, SCAMP5, RAB27A, RNF103, SUSD6, SNX19, RUFY3, SERINC1, RAB11FIP5, SLC19A2, VTI1B, SLCO2B1, SYT7, TMEM204, MTG2, SLC4A4, SLC25A23, NAPG, SRPRB, SEC11C, SLC19A1, SUSD1, TECPR1, PKHD1, IMMT, ARHGAP27, NAPB, TRPV5, TCTN3, TM4SF4, TMEM39B, ADCK2, ARL4D, ACVR1B, GGT1, DPEP1, CUZD1, EVA1A, GHR, FKTN, GP2, SLC2A12, KIFC3, KIRREL2, FA2H, SLC48A1, FCER1G, CYB5A, GOLPH3, DSP, DMXL2, ACSL5, FAM171A1, B4GALT4, B3GNT5, CACNA1B, FPR2, DOK7, FKBP2, CHDH, ADGRF1, CLN6, CREB3L2, EMC10, DGKE, EVI2A, FBXO2, GATM, EPHX1, FZD2, FCGR2B, FXYD6, AQP8, CLMN, CNTFR, CD248, COQ2, COX14, DIAPH1, CDIPT, ELOVL1, WDPCP, GPAT2, FKBP11, FGD2, CA12, CA4, CERS4, FMO3, HHAT, CYP4V2, DGKQ, KCNJ16, LRRC37B, CTDNEP1, FES, DGAT1, LIME1, MAGI1, FPGS, LEPROTL1, MALRD1, MAN1C1, AP3M1, CDK14, FZD5, BRI3, CYP26B1, HTR4, KDELR3, FIBP, ELOVL7, ERBIN, CDC42SE1, CD81, CHPF2, SLC25A13, CRB3, ADAM28, ACE2, ANPEP, CNTNAP3, CFTR, CLDN10, ATL2, CLCA4, CYB561, ALG2, SLC25A31, FXYD2, CMKLR1, BSN, CADPS, ACKR3, CCR4, AQP5, COG8, ANKRD46, ARRDC1, ADGRL1, BBS5, C HST4, ARL4C, AP4E1, ASPHD1, AGPS, C12orf66, ATG9A, ALG14, CHMP2B, CADM1, DAPP1, ARFGAP3, CASQ1, AQP12A, AQP12B, CAVIN2, FGFR4, IBTK, ABCC1, SMPD4, MOXD1, NCEH1, KCNA5, LRFN5, MYZAP, LFNG, NCAM1, GIPC1, NDUFS1, MC4R, GLCE, GFRA2, MCUB, SLC16A9, MSTO1, GNG12, SLC2A6, KIAA2013, KCNK10, SLC16A10, IGFLR1, LYN, FAM234B, NOD2, HPS6, MYOC, ICA1, OR10R2, SLC16A7, NDUFB9, PRKCA, PDLIM4, PSKH1, LRRK2, NDST2,
11. Cancer treated: endometrial cancer
   a. Tissue protected: Colon TRDN, TMED6, TMEM158, UGT2A3, TMEM138, SLC7A6, TMEM60, MAP1LC3A, CD36, MGAT4C, SCGN, SLC14A1, TMEM106B, UBE2J1, SNTG2, TMEM135, PIM1, STBD1, STX1A, ENPP1, SMIM24, SUSD3, SOCS7, NTRK3, RAC1, PCDHGA10, SLC26A1, HCRTR1, PPP1R16B, BVES, LDLR, PILRA, RAC3, MUC12, KLRG1, PARVB, DLC1, PLCB3, PCDHA4, SDR16C5, RDH13, RECK, PTPRT, RAB28, PSCA, KCNH8, MRGPRF, SEL1L3, TMEM45A, SERP1, TPSG1, UPK3BL2, SLC18A1, SPATA9, SPPL3, MAPKAP1, SLC9A3, PTGDR, ADCYAP1R1, SYNGR4, PRKN, ABCB1, MYORG, PLPPR2, PLPP7, SLC8A1, OBSCN, UGT2B4, SERP2, TMEM72, SLC3A1, UGT2B17, VSIG10, SLC7A7, TMEM106C, TMEM143, VAMP5, UTRN, VPS35L, UGT2B28, ZP2, QTRT1, TMEM128, TMEM253, TOMM40L, TBCD, UNC13B, PLAUR, SYTL2, PRRG2, TMX2, UGT2B15, UPK3BL1, SCAMP5, RAB43, SYP, PIGZ, PTPRN2, SLC39A2, SLC39A14, SLC6A15, SNX6, TGFBR1, SLC38A5, SEC11C, PHLPP2, SCN7A, REEP4, MSMO1, ARHGAP27, NAPB, TSNARE1, TRAF3IP3, TMEM154, TYRO3, UBL3, TMEM39B, CDHR5, EFHD1, DPEP1, EMC9, GSDMB, IGSF8, CX3CR1, FPR1, GRAP, PHKA1, ABCG1, CYP2S1, ABCG2, GPC5, CD177, CLCA1, DAB2, GLP2R, FPR2, CADM4, ENTPD7, RASGRP2, CLN6, ENOX2, CALN1, DMTN, AQP8, CHODL, CA4, DEPDC5, GDAP1, DGKQ, IGSF11, CPTP, HSD17B2, LIME1, SLC7A8, LPP, MAN1C1, ABHD16A, CXCR1, EPHA10, FER1L6, ANO8, ANKRD13B, AQP1, BEST2, BRI3, CDC42EP5, MPP1, GAL3ST2, B3GNT8, KDELR3, FAM162A, DENND5A, ACE2, SH3BP5, CNTNAP3, CDH17, CD300LF, AFG1L, ENPEP, SLC25A31, CLDN11, KCNIP3, ATP1A2, CADPS, CD300A, ADGRG6, B3GNT6, CEACAM6, TMEM30B, DCXR, ACKR2, AP1S3, CMTM8, EPB41L3, ABHD12, BCL2L13, C12orf66, CNPPD1, COMTD1, HSD3B7, ALG14, CEACAM3, B3GAT1, CASQ1, PYCARD, CYP2C9, DIP2A, HMOX1, SLC16A6, KCNN3, MS4A18, GPR153, MS4A12, GFRA2, MCUB, KIAA2013, LY9, GPA33, DPY19L1, ALOX12, MUC13, NOD2, OR10R2, MRC1, KLRC3, MAPK8IP1,
   b. Tissue protected: Lung VSIG4, SLC30A4, SLC7A6, CD300C, SLC14A1, TRIM13, UBE2J1, VIPR2, SLC22A15, SLC25A17, TGFBR3, SUSD3, SLC26A1, NUS1, PPP1R16B, BVES, AGER, SLC18A2, PCDH10, SDR16C5, RECK, PTPRT, RAB28, PTPRG, MGAT4B, EHD2, OBSCN, VAMP5, UTRN, QTRT1, PRRG2, RHOG, PSD4, RAB43, SLC39A2, SLC6A15, STEAP1, PHLPP2, SCN7A, REEP4, SIGLEC9, UBL3, EHD1, GJB4, EHD4, FPR1, CD247, CCR7, CYB5A, ABCG1, CAVIN1, GPC5, DAB2, AOC3, ECEL1, CLDN18, CALN1, HCLS1, DMTN, CCDC88A, AIFM2, CLEC2B, FES, LIME1, CLIC2, CAPN2, CXCR1, ANKRD13B, AQP1, DGKZ, ABCA8, APBB1IP, FAM162A, FCAR, ELMO2, CAV1, ENPEP, CACNA2D2, ACE, AP3S1, BSN, CAV2, AP3S2, CEACAM6, CMTM8, COMTD1, BDKRB2, CXCR3, LAMP3, EHD3, MAP1LC3B, NSMF, LRRK2,
   c. Tissue protected: Skin TRDN, TMED6, SUN3, TMEM138, CLEC10A, CD300C, MGAT4C, PERP, VIPR2, RXFP1, SOX10, SNTG2, TMEM135, SLC25A17, TGFBR3, ENPP1, TMPRSS11D, SLC6A1, RAC1, SLC26A1, NEGR1, NUS1, PPP1R16B, SLC26A9, RAC3, TMEM40, MTMR2, SDR16C5, RDH13, RAB28, KCNH8, LTBR, TMEM45A, SERP1, MGAT4B, TYROBP, SLC10A6, MAPKAP1, SLC9A3, LANCL2, PLPP7, LAX1, LYPD3, KRT1, SLC8A1, DMPK, OBSCN, SERP2, TRPM2, TMEM79, SLC7A7, YRDC, VAMP5, UTRN, ZP2, TMEM128, TBCD, PRRG2, TMX2, UGT2B15, S1PR5, SLC39A2, NGFR, SLC38A5, LY6D, REEP4, MSMO1, NAPB, RIPK4, TSNARE1, SIGLEC9, TRAF3IP3, TYRO3, TMEM39B, EMC9, CYBA, GJB4, FFAR2, IGSF8, FA2H, FPR1, GRAP, PHKA1, CYP2W1, ABCG1, DSP, CAVIN1, GPC5, CD177, DAB2, GLP2R, ANO7, FYN, FPR2, ENOX2, ATRAID, CALN1, DMTN, FAT2, CAPNS2, HCN1, MALL, GDAP1, DGKQ, DSC3, SLC7A8, LPP, EPHA10, B3GNT4, DSG1, ABCA8, BRI3, KDELR3, BST1, CAV1, AFG1L, SLC25A31, MS4A1, CAV2, CAV3, CD1D, ASPRV1, TMEM63B, DSG3, TMEM30B, DCXR, ACKR2, BCL2L13, CLEC2A, CNPPD1, COMTD1, ATP12A, HSD3B7, GPR153, MCUB, MAP1LC3B, ALOX12, NOD2, CHL1, OR10R2, NCS1, TRDN, SLC30A4, TRIM13, UBE2J1, ULBP3, SUSD3, TYR, RHBG, DLC1, PLN, PTPRT, SEL1L3, ICAM3, MYORG, PLPPR2, TMEM143, QTRT1, DCT, UNC13B, PRNP, RHCG, SH2D3C, RHOG, S100A8, PMEL, SEMA5B, PHLPP2, ARHGAP27, EHD1, GSDMC, EHD4, CYB5A, TM7SF2, CYB561A3, FCGR2B, FAM210A, GPAT2, CD82, DNER, FGD2, IGSF11, CLEC2B, CPTP, CXCR1, DGKZ, PROCR, B3GNT8, FAM162A, ELMO2, AP3S1, CDH13, AP3S2, CMTM8, ATG2A, BDKRB2, ALG14, ADGRA1, PYCARD, MPP2, HAS3, LHFPL6, MUC21, GFRA2, EHD3, KIT, KIAA0040, MAPK8IP1, ,
   d. Tissue protected: Liver VNN1, TRDN, TMEM138, MAP1LC3A, CLEC10A, CD300C, CREB3L3, SLC14A1, TRIM13, UBE2J1, TMEM135, SLC25A17, PIM1, STBD1, SLC6A4, ENPP1, SUSD3, SLC25A40, SLCO1B3, SLCO1B1, NTRK3, SLC27A5, SLC26A1, NEGR1, PPP1R16B, LDLR, SLC18A2, KSR2, MTMR2, PTGDR2, RDH13, RECK, PTPRT, RDH16, LIN7A, SEL1L3, TMEM45A, SERP1, MGAT4B, SPPL3, MAPKAP1, SYNGR4, PRRT2, ABCB1, NAT8, MYORG, PLPPR2, PNPLA3, SLC8A1, MME, OBSCN, UGT2B4, SERP2, SLC3A1, TFR2, TMEM106C, VAMP5, UTRN, VPS35L, TMEM128, TNS2, TBCD, UNC13B, TMX2, UGT2B15, ST7L, RAB43, SLC39A2, SLC39A14, TGFBR1, SLCO2B1, MSMO1, NAPB, TSNARE1, SIGLEC9, TYRO3, CDHR5, GSDMB, EVA1A, GPR37, GLRB, CX3CR1, CYP2W1, CYP2D6, CYB5A, ABCG1, CYP2S1, GJA5, CD177, CACNA1A, FYN, FPR2, GAS2, CADM4, SLC2A2, ENTPD7, RASGRP2, CREB3L2, CYP2A7, HEPACAM, CYP1A2, DMTN, CYP3A4, FAM210A, CYP2A6, CYP2B6, FMO3, CYP4V2, DEPDC5, MALL, ABCB4, NINJ1, CYP2C8, DHCR24, CLEC2B, FES, HSD17B2, LIME1, MAL2, MAN1C1, CYP2C19, BCL2L10, AQP9, ABCA8, BRI3, AFG1L, ENPEP, ASGR1, ADRA2B, ABCB11, ATP1A2, CYP2A13, TMEM63B, TMEM30B, DCXR, CMTM8, BACE2, ASGR2, ATG2A, PIK3AP1, CYP3A43, HSD3B7, B3GAT1, CASQ1, CYP2C9, MADCAM1, SLC16A6, MFAP3L, LIMS2, GPR15, DPY19L1, SLC2A8, ALOX12, OR10R2, ABCC2, LRRK2, SLC10A1, NPC1L1,
   e. Tissue protected: Kidney (glomeruli) ZDHHC16, TMEM138, ZAP70, MGAT4C, SLC14A1, UBE2J1, VIPR2, KDR, S LC22A15, TMEM135, SLC25A17, TGFBR3, SUSD3, SLC45A1, RAC1, PILRA, RAC3, KSR2, PARVB, PCDH10, RECK, PTPRT, RAB28, PTPRG, MRGPRF, SEL1L3, SERP1, SLC34A1, TPSG1, PTGDR, ADCYAP1R1, PLA2R1, PRKN, PTH2R, PNPLA3, NOS3, EHD2, MME, NKD1, NPHS2, SERP2, VAMP5, UTRN, VPS35L, TSPAN7, TNS2, PRRG2, STK10, SNCA, PSD4, SNX6, SLC38A5, PITPNM3, PTPRO, SREBF1, SCN7A, RTL1, NAPB, TYRO3, EFHD1, EHD1, EVA1A, HYAL2, CX3CR1, EHD4, CAVIN1, GPC5, DAB2, FYN, GAS2, CD93, CD34, RASGRP2, CALN1, DMTN, ENG, DOCK5, FAM210A, FGD2, CLEC2B, LIME1, LPP, ADGRL4, CLIC2, CAPN2, ANO8, AQP1, ELMO2, CAV1, AFG1L, ENPEP, BSN, CAV2, CDH13, BBS7, B3GAT1, HAS3, SLC43A2, LIMS2, GPR153, ITGA8, EHD3, MLIP, MAP1LC3B, NPHS1, KIAA0040, PDGFRA,
   f. Tissue protected: Kidney (tubules) VNN1, TRDN, TMED6, SUN3, TRPM3, TMEM138, TBC1D3G, TREH, MGAT4C, THY1, SLC14A1, UBE2J1, VIPR2, SLC22A15, SELPLG, TMEM135, TMEM240, PIM1, SLC6A4, ENPP1, SMIM24, SLC5A12, SLC5A2, SUSD3, STIMATE, SLC12A1, SLC25A40, SLC4A10, SCNN1D, SLC6A19, SLC22A11, SLC34A3, NTRK3, SLC26A1, SLC28A2, HCRTR1, RDH11, NEGR1, PPP1R16B, PTH1R, LDLR, AGER, RHBG, PARVB, RHBDD2, DLC1, PLCB3, PCDHA4, PCDH10, PDE2A, RDH13, RECK, PTPRT, RALB, RAB28, PSCA, KCNH8, LIN7A, MRGPRF, SEL1L3, LTBR, TMEM45A, SERP1, MGAT4B, SLC34A1, UPK3BL2, SPPL3, MAPKAP1, SLC9A3, PTGDR, ADCYAP1R1, NOS1, PRRT2, PRKN, ABCB1, NAT8, MYORG, PLPPR2, PNPLA3, PLPP7, SLC8A1, NTRK2, EHD2, MME, OBSCN, NKAIN4, RFTN2, UGT2B4, SLC22A6, SERP2, TMEM72, TRPM2, UGT3A1, XPNPEP2, SLC3A1, VSIG10, ATP6V0A4, SLC7A7, TMEM106C, VAMP5, UTRN, VPS35L, ZP2, QTRT1, TMEM252, TMEM128, TMEM174, TOMM40L, TBCD, UNC13B, PLAUR, RHCG, SYTL2, PRRG2, TMPRSS2, TMX2, UPK3BL1, SLC28A1, SEMA6B, SIRT2, SLC22A2, SH2D3C, RHOG, SCAMP5, RAB43, SLC22A8, SLC13A3, PIGZ, SLC39A2, SLC39A14, SNX6, SLC6A18, SLC13A2, TGFBR1, TMEM213, SLC38A5, SLC5A1, SLC4A4, SEC11C, SREBF1, REEP4, SLC35G2, MSMO1, OPRD1, NAPB, RIPK4, TSNARE1, TYRO3, TSPAN16, UBL3, TMEM39B, CDHR5, ENPP6, EFHD1, IYD, DPEP1, EMC9, CUBN, CLSTN2, EHD1, GSDMB, EVA1A, IGSF8, HYAL2, GLRB, GSDMC, KIRREL2, CX3CR1, EHD4, GRAP, CKMT2, PHKA1, CPNE6, CYB5A, ABCG1, CLTRN, CYP2S1, GJA5, GPC5, CD177, DAB2, FAM151A, CPT1C, GAS2, CADM4, SLC2A2, DOCK2, CLCNKB, ADGRG5, CLCNKA, CLN6, CREB3L2, ENOX2, TM7SF2, ATRAID, CASR, DMTN, FAM210A, FRRS1L, CYP2B6, CA4, FMO3, CYP4V2, DEPDC5, GDAP1, GLIPR1L2, LPXN, DGKQ, KIAA0319, IGSF11, DHCR24, CLEC2B, FES, CPTP, LIME1, SLC7A8, MAL2, LPP, MAN1C1, EPHB1, ABHD16A, BSND, BCL2L10, CAPN2, CXCR1, EPHA10, ANO8, AQP6, AQP1, DSG1, ABCA8, BRI3, CDC42EP5, NPR3, MPP1, FAM162A, CDC42SE1, DENND5A, SH3BP5, BST1, AFG1L, ENPEP, SLC7A9, DUSP15, SLC25A31, FXYD2, ACE, AP3S1, CATIP, BSN, CLDN2, CAV3, AP3S2, TMEM63B, DCXR, ACKR2, ANO5, AP1S3, CMTM8, EPB41L3, C12orf66, COMTD1, ATP12A, PIK3AP1, CYP3A43, HSD3B7, B3GAT1, CASQ1, PYCARD, DIP2A, MPP2, HMOX1, LRFN2, HAS3, SLC43A2, SLC16A6, KCNJ10, KCNJ12, MFAP3L, GFRA2, MCUB, EHD3, MLIP, KIAA2013, MAP1LC3B, FMO1, DPY19L1, LRP2, ALOX12, NPHS1, KIAA0040, CHL1, NXPE2, IL23R, OR10R2, MRC1, GRIN2D, NSMF, MAPK8IP1, ABCC2, KCTD8, LRRK2, NPC1L1,
   g. Tissue protected: Heart (cardiomyocytes) TMED6, SLC30A4, SPAG4, TMEM138, SLC7A6, UCP3, CD300C, CD36, MGAT4C, RASL10B, SLC14A1, TMEM106B, VIPR2, SLC22A15, SLC25A17, TGFBR3, STBD1, SUSD3, SLC4A10, NTRK3, RAC1, NEGR1, NUS1, PPP1R16B, BVES, LDLR, RAC3, PARVB, POPDC2, PCDH10, RECK, PLCH2, PLN, RAB28, MRGPRF, SEL1L3, SERP1, SHISA4, INPP5D, MGAT4B, FLT1, SPATA9, SGCG, SRL, MAPKAP1, SLC9A3, PRRT2, PRKN, LANCL2, ART3, MYORG, PLPP7, SLC8A1, DMPK, EHD2, OBSCN, RFTN2, SERP2, SIPA1, SLC7A7, YRDC, TMEM106C, TMEM143, VAMP5, TREML1, UTRN, VPS35L, ZP2, QTRT1, TOMM40L, TBCD, PRRG2, TMX2, SEMA6B, SIRT2, SH2D3C, SCAMP5, PIGZ, SLC39A2, SLC39A14, RYR1, SLAMF8, SNX6, TGFBR1, SLCO2B1, SEMA5B, ST8SIA2, SCN7A, SLC35G2, MSMO1, NAPB, TSNARE1, TYRO3, UBL3, TMEM39B, EMC9, EHD1, GSDMB, EVA1A, MMP27, CX3CR1, EHD4, FPR1, GRAP, CKMT2, PHKA1, CYP2W1, ABCG1, CYP2S1, DSP, ABCG2, CAVIN1, GPC5, CDH15, ADRA1A, CADM4, RASGRP2, CAVIN4, CLN6, CREB3L2, TM7SF2, HHATL, FAM168B, CALN1, CYB561A3, FCGR2B, CHRNA1, GPAT2, FGD2, CYP4V2, DEPDC5, MALL, DGKQ, CAMK2B, IGSF11, CLEC2B, FES, MBOAT7, CPTP, LIME1, L EPROTL1, LPP, MAN1C1, ADGRL4, CLIC2, ABHD16A, CXCR1, EPHA10, FCGRT, ANO8, ANKRD13B, B3GNT4, DGKZ, B3GNT8, CDC42SE1, ATP1A3, DENND5A, CNTNAP3, BST1, CAV1, AFG1L, SLC25A31, AP3S1, ATP1A2, BSN, CADPS, CAV2, CAV3, CD300A, CDH13, CD1D, AKAP6, ADGRG6, ADRB1, AP3S2, TMEM30B, CFC1, DCXR, ANO5, AP1S3, CMTM8, BCL2L13, BACE2, CNPPD1, COMTD1, HSD3B7, CASQ1, SLC16A6, MFAP3L, NCAM1, GPR182, LIMS2, MCUB, EHD3, GPR15, MLIP, DPY19L1, SLC2A8, ALOX12, NOD2, OR10R2, MCEMP1, DES, LRRK2,
   h. Tissue protected: Thyroid TMED6, TMEM158, SLC30A4, TMEM138, TMEM106B, TRIM13, UBE2J1, VIPR2, SLC22A15, TMEM135, SLC25A17, TGFBR3, PIM1, SLITRK4, ENPP1, SCNN1D, NTRK3, NEGR1, NUS1, PPP1R16B, KCNQ5, BVES, TPO, TMEM40, RHBDD2, PCDH10, RDH13, RAB28, LIN7A, SEL1L3, SERP1, INPP5D, UPK3BL2, MAPKAP1, PRKN, LANCL2, PSD3, PLPP7, SLC8A1, NTRK2, OBSCN, SERP2, TRPM2, SLC7A7, YRDC, TMEM106C, UTRN, VPS35L, QTRT1, TMEM128, TBCD, UNC13B, PRRG2, UPK3BL1, RTP5, RAB43, SLC39A2, SLC39A14, RNFT2, SLCO2B1, SREBF1, REEP4, MSMO1, TSNARE1, SIGLEC9, TYRO3, UBL3, TMEM39B, C19orf18, EFHD1, IYD, DUOX1, EMC9, EHD1, EVA1A, GPR37, CX3CR1, EHD4, GRAP, PHKA1, CYP2W1, ABCG1, CYP2S1, ABCG2, CAVIN1, GPC5, DAB2, FYN, FPR2, CADM4, CLCNKB, RASGRP2, CLCNKA, CLN6, CREB3L2, FAM168B, ATRAID, CALN1, DMTN, FCGR2B, FAM210A, GPAT2, FGD2, HCN1, FMO3, DEPDC5, MALL, GDAP1, LPXN, DGKQ, CAMK2B, IGSF11, FES, LIME1, ADGRL4, CLIC2, EPHB1, CAPN2, CXCR1, EPHA10, FER1L6, ANO8, ANKRD13B, PROCR, DENND5A, ELMO2, AAK1, CAV1, AFG1L, ENPEP, CRHR1, CAV2, CD300A, TMEM63B, TMEM30B, DCXR, ACKR2, AP1S3, CMTM8, C12orf66, COMTD1, HSD3B7, CEACAM3, PYCARD, MPP2, NCAM1, GFRA2, GPR15, MAP1LC3B, OR10R2, GRIN2D, MAPK8IP1, NCS1,
   i. Tissue protected: Pancreatic endocrine cells (Islets) SLC30A4, MAP1LC3A, SCGN, UBE2J1, SLC25A17, TGFBR3, PIM1, STX1A, RAB3A, RTN1, SCG3, HCRTR1, NEGR1, POMGNT2, PTGDR2, RDH13, PTPRT, SERP1, UPK3BL2, SPPL3, MAPKAP1, PTPRN, PRKN, LANCL2, PTH2R, MYORG, SLC8A1, OBSCN, NECAB2, SLC17A6, SERP2, TRPM2, YRDC, UTRN, TSPAN7, TNS2, UNC13B, PLAUR, SLC30A8, PRNP, PRRG2, UPK3BL1, STK10, SCAMP5, SYP, PTPRN2, SLC39A2, SLC38A5, SLCO2B1, MAPK10, SLC35G2, MSMO1, OPRD1, NAPB, TSNARE1, SIGLEC9, TMEM39B, FFAR2, DGCR2, PHKA1, CYP2W1, CLTRN, GNAO1, CREB3L2, CASR, AQP8, CD82, CYP2B6, CYP4V2, GDAP1, DGKQ, IGSF11, SLC7A8, CAPN2, CXCR1, EPHA10, DGKZ, ENO2, ELMO2, AFG1L, FXYD2, AMPH, ATP1A2, CADPS, CRHR1, CD1D, TMEM30B, CFC1, AP1S3, EPB41L3, BBS7, GAD2, ALOX5, MPP2, SLC16A6, GPR153, GFRA2, KIAA2013, MAP1LC3B, FMO1, OR10R2,
   j. Tissue protected: Pancreatic exocrine cells TMED6, TMEM138, CLEC10A, CD300C, RASL10B, TBC1D3D, UBE2J1, SELPLG, PIM1, ENPP1, STIMATE, SLC4A10, NTRK3, SLC26A1, PPP1R16B, POMGNT2, TMEM40, PCDHA4, RDH13, PTPRT, PSCA, KCNH8, MRGPRF, SEL1L3, SERP1, UPK3BL2, SPATA9, PRKN, PTH2R, NEURL1, MYORG, PLPP7, SLC8A1, OBSCN, SERP2, TMEM72, TRPM2, UGT3A1, SLC3A1, VSIG10, YRDC, TMEM106C, VAMP5, UTRN, UNC13D, TMEM128, UNC13B, PLAUR, TBC1D3E, SYTL2, TMPRSS2, TMX2, UPK3BL1, SYCN, SCAMP5, SLC39A2, SLC39A14, SLC38A5, SLCO2B1, SLC4A4, SEC11C, SCN7A, REEP4, ARHGAP27, NAPB, TSNARE1, TRAF3IP3, TMEM39B, EFHD1, DPEP1, EMC9, CUZD1, EVA1A, GLRB, KIRREL2, FA2H, GRAP, CYB5A, ABCG1, DSP, DAB2, FPR2, RASGRP2, CLN6, CREB3L2, FCGR2B, AQP8, FAM210A, GPAT2, FGD2, CA4, FMO3, CYP4V2, MALL, DGKQ, IGSF11, FES, LIME1, LEPROTL1, MAN1C1, EPHA10, AQP1, BRI3, KDELR3, CDH9, FAM162A, CDC42SE1, DENND5A, ACE2, CNTNAP3, AFG1L, SLC25A31, FXYD2, BSN, CADPS, CAV2, CD300A, TMEM30B, AP1S3, CMTM8, C12orf66, COMTD1, PIK3AP1, HSD3B7, ALG14, BBS7, CASQ1, AQP12A, AQP12B, PYCARD, MPP2, NCAM1, GFRA2, MCUB, KIAA2013, NOD2, OR10R2, MAPK8IP1, LRRK2,
12. Cancer treated: breast cancer
   a. Tissue protected: Colon TTYH3, ZDHHC2, UGT2A3, TMEM138, TMEM60, MS4A8, C2CD2, FCAMR, RMDN2, SNTG2, SYT13, STX1A, LPCAT2, SLC26A2, PITPNM1, SOCS7, PCDHB5, NDUFS8, SLC19A3, MGAM, ARHGAP5, PLPP1, MUC12, VPS26B, WHAMM, PTPRD, ITGB1, KLRG1, DLC1, PLCB3, SLC25A20, PVR, RAB3B, PSCA, KCNH8, SEL1L3, TMEM45A, TPSG1, TRPM7, UPK3BL2, SPPL3, STYK1, TMEM134, MAPKAP1, SLC9A3, IFI27, ITLN1, PTGDR, SYNGR4, PEX10, OSBPL7, ABCB1, LRRC55, PLPP7, OBSCN, PLOD1, NUP54, UGT2B4, TMEM72, UGT2B17, VSIG10, SYT11, SLC7A7, TMEM143, UGT2B28, ZP2, QTRT1, TMEM69, SYTL3, TMEM253, PLAUR, UGT2B15, UPK3BL1, SLC22A1, SEZ6, SCAMP5, SYP, PIGZ, SLC39A2, SLC39A14, SLC6A15, SNX6, SDCBP2, SEC11C, SH3KBP1, PHLPP2, REEP4, SLC35B1, TMEM171, VASN, TCTN3, ATG2B, CRK, CDHR5, DPEP1, HTR7, GSDMB, FGFR2, IGSF8, SLC2A12, KIFC3, FPR1, ABCG1, ABCG2, ACSL5, CD177, CLCA1, GLP2R, CEACAM1, GBA2, FPR2, RASGRP2, HTR1A, ENOX2, GPR34, DMTN, CEACAM5, CHODL, CA4, GDAP1, GNG11, IGSF11, CPTP, HSD17B2, SLC7A8, FADS3, EPHA10, FER1L6, ANKRD13B, AQP1, BEST2, BRI3, CDC42EP5, MPP1, GAL3ST2, B3GNT8, KDELR3, CDHR2, ACE2, SH3BP5, CNTNAP3, CDH17, ENPEP, CLDN11, KCNIP3, CADPS, C1orf210, ORAI1, ADGRG6, ASIC5, DCXR, ACKR2, ARHGEF4, EPB41L2, ABHD12, BCL2L13, CDH26, BLNK, CYP2C9, DIP2A, IBTK, ABCC5, HMOX1, GRB14, GPR153, MS4A12, SLC16A9, KIAA2013, ITGB4, LY9, GPA33, LZTR1, MUC13, NFXL1, LBR, OR10R2, MRC1, NDUFB9,
   b. Tissue protected: Lung SLC30A4, LRP1, CD300C, VIPR2, RMDN2, SLC22A15, SLC25A17, LPCAT2, SLC19A3, NUS1, AGER, TRPV6, SLC18A2, WHAMM, SLC25A20, REEP2, PTPRG, MGAT4B, SGIP1, OSBPL7, KIRREL3, EHD2, OBSCN, QTRT1, P2RY2, SEZ6, RHOG, SLC39A2, SLC6A15, STEAP1, RYK, PHLPP2, REEP4, ATG2B, HLA-DRB1, EHD1, FGFR2, EHD4, FPR1, CD247, CCR7, ABCG1, CAVIN1, ACSL5, GBA2, CLDN18, GPR34, HCLS1, DMTN, CEACAM5, CCDC88A, AIFM2, FES, CLIC2, ANKRD13B, AQP1, ABCA8, AIG1, APBB1IP, DGKG, FCAR, ELMO2, CAV1, AQP4, ENPEP, CACNA2D2, ACE, CAV2, ANXA1, EPB41L2, BDKRB2, CXCR3, CAVIN2, IBTK, GRB14, LAMP3, EHD3, MMP2, GALNT13, NSMF,
   c. Tissue protected: Skin TMEM243, TTYH3, ZDHHC2, SUN3, TMEM138, C2CD2, CLEC10A, CD300C, ACER3, PERP, VIPR2, SOX10, SNTG2, SLC25A17, TMPRSS11D, SLC6A1, PITPNM1, PCDHB5, TMEM97, ARHGAP5, NEGR1, NUS1, NTM, TMEM40, VPS26B, REEP2, KCNH8, GPIHBP1, TMEM45A, MGAT4B, TYROBP, STYK1, TMEM134, SLC10A6, MAPKAP1, SLC9A3, IFI27, LRRC55, PLPP7, LAX1, KRT1, PLA2G4E, OBSCN, RMC1, SLC7A7, SRC, ZP2, TMEM69, SYTL3, TACSTD2, UGT2B15, P2RY2, SLC22A1, SLC39A2, SDCBP2, RYK, SH3KBP1, SLC19A1, LY6D, REEP4, RIPK4, TMEM171, CRK, COL17A1, CYBA, FGFR2, IGSF8, FA2H, FPR1, ABCG1, DSP, CAVIN1, CDH6, CD177, GLP2R, CACNA2D1, GBA2, FPR2, ENOX2, GPR34, DMTN, FAT2, CD99, CD248, HCN1, GDAP1, DEF6, DSC3, FGFBP1, SLC7A8, FADS3, EPHA10, B3GNT4, DSG1, ABCA8, BRI3, KDELR3, CAV1, CCR2, MS4A1, CAV2, CAV3, CD1D, ANXA1, ASPRV1, C1orf210, ORAI1, ASIC5, DSG3, DCXR, ACKR2, BCL2L13, ATP12A, BLNK, IBTK, ABCC1, ABCC5, GPR153, SLC16A9, TACR1, ITGB4, OR10R2, NDUFB9, NCS1, TMEM243, SLC30A4, ZDHHC6, MS4A8, SLC2A1, RMDN2, SYT13, LPCAT2, RETSAT, RHBG, DLC1, PLN, SEL1L3, TECTA, SGIP1, OSBPL7, NUP54, TMEM143, QTRT1, DCT, RHCG, RHOG, PMEL, SEMA5B, PHLPP2, TCTN3, EHD1, GSDMC, KIFC3, EHD4, IGSF3, HLA-DPA1, GSG1L, CYB561A3, FCGR2B, CEACAM5, GPAT2, DNER, FGD2, IGSF11, CPTP, B3GNT8, ELMO2, CDH13, EPB41L2, ATG2A, BDKRB2, ADGRA1, CAVIN2, LHFPL6, MUC21, EHD3, KIT, LZTR1, ,
   d. Tissue protected: Liver TTYH3, TMEM138, LRP1, C2CD2, CLEC10A, CD300C, CREB3L3, FCAMR, RMDN2, SYT13, SLC25A17, LPCAT2, SLC25A40, SLCO1B3, SLCO1B1, NDUFS8, SLC25A43, SLC27A5, SLC19A3, RETSAT, NEGR1, SLC18A2, PTPRD, KSR2, PTGDR2, SLC25A20, LIN7A, SEL1L3, TMEM45A, MGAT4B, SPPL3, MAPKAP1, SYNGR4, PRRT2, ABCB1, LRRC55, NAT8, KIRREL3, MME, OBSCN, UGT2B4, TFR2, UGT1A6, ZDHHC13, UGT2B15, ST7L, SLC22A1, SLC39A2, SLC39A14, SLCO2B1, SDCBP2, TEX261, TMEM171, VASN, CRK, CDHR5, GSDMB, FGFR2, EVA1A, GPR37, KIFC3, CYP2D6, ABCG1, GJA5, ACSL5, CD177, CACNA2D1, CEACAM1, GBA2, CACNA1A, FPR2, GAS2, SLC2A2, RASGRP2, HTR1A, CREB3L2, CYP2A7, GPR34, FOLH1, CYP1A2, DMTN, CYP3A4, CEACAM5, CYP2B6, FMO3, GPRC5B, NINJ1, CYP2C8, CDH2, FES, HSD17B2, MAL2, CYP2C19, FADS3, BCL2L10, AQP9, ABCA8, AIG1, BRI3, CDHR2, ENPEP, ASGR1, ABCB11, CTSL, C1orf210, ASIC5, ACAD11, DCXR, HSD11B1, ASGR2, ATG2A, PIK3AP1, CAVIN2, CYP2C9, IBTK, GRB14, MADCAM1, LIMS2, SLC16A9, GPR15, ITGB4, SLC2A8, LZTR1, OR10R2, NDUFB9, ABCC2, DPP4, NPC1L1,
   e. Tissue protected: Kidney (glomeruli) ZDHHC16, TMEM138, ZDHHC6, ACER3, VIPR2, KDR, RMDN2, SLC22A15, SYT13, SLC25A17, SLC45A1, PCDHB5, SLC19A3, MGAM, PODXL, KIRREL1, PLPP1, TRPV6, WHAMM, ITGB1, KSR2, PVR, PTPRG, SEL1L3, TPSG1, SCUBE1, TECTA, PTGDR, PLA2R1, PTH2R, LRRC55, KIRREL3, MAP6, NOS3, EHD2, MME, NKD1, NPHS2, RMC1, ZC3H12A, TSPAN7, TTC7B, SLC22A1, SNCA, SNX6, SDCBP2, RYK, PITPNM3, PTPRO, SLC35B1, TMEM171, VASN, HTR7, EHD1, FGFR2, EVA1A, HYAL2, EHD4, CAVIN1, CDH6, CEACAM1, GAS2, CD93, FAM107A, CD34, RASGRP2, HTR1A, GPR34, DMTN, ENG, CD99, CD248, FGD2, GPRC5B, MAS1, ADGRL4, CLIC2, FADS3, AQP1, ELMO2, CAV1, ENPEP, CHRM1, CAV2, CDH13, ANXA1, EPB41L2, BBS7, ABCC1, GRB14, SLC43A2, LIMS2, GPR153, ITGA8, SLC16A9, EHD3, MLIP, MMP2, LZTR1, NFXL1, IKBIP, DYSF, DPP4, PDGFRA,
   f. Tissue protected: Kidney (tubules) TMEM243, TTYH3, ZDHHC2, SUN3, TRPM3, TMEM138, ZDHHC6, TBC1D3G, TREH, MS4A8, C2CD2, ACER3, FCAMR, THY1, VIPR2, RMDN2, SLC22A15, TMEM240, SLC5A12, SLC12A1, PITPNM1, SLC25A40, PCDHB5, SLC6A19, S1PR2, NDUFS8, SLC34A3, SLC19A3, MGAM, ARHGAP5, SLC28A2, RDH11, RETSAT, NEGR1, KIRREL1, PLPP1, AGER, RHBG, VPS26B, VHL, WHAMM, PTPRD, DLC1, PLCB3, PDE2A, SLC25A20, PVR, RALB, PSCA, KCNH8, KCNJ1, LIN7A, SEL1L3, TMEM45A, UCHL1, MGAT4B, TRPM7, UPK3BL2, TMEM132E, PRICKLE1, SPPL3, STYK1, TMEM134, MAPKAP1, SLC9A3, IFI27, PTGDR, SGIP1, NOS1, PEX10, PRRT2, OSBPL7, ABCB1, LRRC55, NAT8, PLPP7, KIRREL3, EHD2, MME, OBSCN, PLOD1, NUP54, RFTN2, UGT2B4, SLC22A6, TMEM72, UGT3A1, VSIG10, A TP6V0A4, SLC7A7, SRC, ZC3H12A, ZP2, QTRT1, UGT1A6, TMEM252, SYTL3, TMEM174, TTC7B, PLAUR, ZDHHC13, RHCG, TMPRSS2, FASLG, UPK3BL1, SLC28A1, SLC22A1, SLC22A2, RHOG, SCAMP5, SLC22A8, SLC13A3, PIGZ, SLC39A2, SLC39A14, SNX6, SLC6A18, SLC13A2, TMEM213, SLC5A1, SLC4A4, SDCBP2, RYK, SEC11C, TEX261, REEP4, SLC35G2, RIPK4, TMEM171, VASN, ATG2B, CRK, CDHR5, ENPP6, EMC4, IYD, DPEP1, ENPP3, CUBN, CLSTN2, EHD1, GSDMB, FGFR2, EVA1A, IGSF8, HYAL2, GSDMC, KIFC3, KIRREL2, EHD4, CKMT2, ABCG1, CLTRN, GJA5, ACSL5, CDH6, CD177, FAM151A, GBA2, GAS2, FAM107A, FNDC5, GSG1L, SLC2A2, HTR1A, ADGRG5, CREB3L2, ENOX2, FAM169A, GPR34, CASR, FOLH1, DMTN, CD248, CLIC4, CYP2B6, CA4, FMO3, GDAP1, GNG11, GLIPR1L2, KIAA0319, GPRC5B, IGSF11, CDH2, FES, CPTP, SLC7A8, MAL2, FADS3, BSND, BCL2L10, EPHA10, AQP6, AQP1, DSG1, ABCA8, AIG1, BRI3, CDC42EP5, NPR3, MPP1, CDC42SE1, CDHR2, ADAM28, SH3BP5, CCR2, ENPEP, DUSP15, FXYD2, ACE, CLDN2, CAV3, CPT1B, COX7B, CTSL, C1orf210, A SIC5, DCXR, ACKR2, ARHGEF4, EPB41L2, ATP12A, BLNK, PIK3AP1, CDH16, DIP2A, IBTK, ABCC5, HMOX1, LRFN2, GRB14, SLC43A2, KCNJ10, KCNJ12, KCNK5, SLC16A9, EHD3, MLIP, KIAA2013, LZTR1, NFXL1, NXPE2, IL23R, OR10R2, MRC1, NDUFB9, NSMF, IKBIP, ABCC2, KCTD8, DYSF, DPP4, NPC1L1,
   g. Tissue protected: Heart (cardiomyocytes) TTYH3, SLC30A4, SPAG4, TMEM138, ZDHHC6, UCP3, CD300C, VIPR2, RMDN2, SLC22A15, SYT13, SLC25A17, LPCAT2, PITPNM1, PCDHB5, SGCB, NDUFS8, SLC19A3, RETSAT, NEGR1, NUS1, VPS26B, WHAMM, PTPRD, P2RY6, SLC25A20, PLN, PVR, GPIHBP1, SEL1L3, MGAT4B, SGCG, SRL, MAPKAP1, SLC9A3, TECTA, SGIP1, PRRT2, OSBPL7, LRRC55, ART3, PLPP7, EHD2, OBSCN, RFTN2, SIPA1, SLC7A7, SRC, TMEM143, TREML1, ZC3H12A, ZP2, QTRT1, TTC7B, ZDHHC13, SLC22A1, SCAMP5, RAB11FIP4, PIGZ, SLC39A2, SLC39A14, RYR1, SLAMF8, SNX6, SLCO2B1, SEMA5B, TEX261, SLC35B1, SLC35G2, TMEM171, VASN, TCTN3, ATG2B, CRK, EHD1, GSDMB, FGFR2, EVA1A, EHD4, FPR1, CKMT2, ABCG1, DSP, ABCG2, CAVIN1, CDH6, CACNA2D1, CDH15, GBA2, ADRA1A, FNDC5, RASGRP2, HTR1A, CREB3L2, HHATL, FAM168B, GPR34, CYB561A3, FCGR2B, CHRNA1, CD248, GPAT2, FGD2, CAMK2B, GPRC5B, IGSF11, CDH2, FES, CPTP, LEPROTL1, ADGRL4, CLIC2, FADS3, EPHA10, FCGRT, ANKRD13B, B3GNT4, AIG1, B3GNT8, DGKG, CDC42SE1, CDHR2, ADAM28, CNTNAP3, CAV1, CCR2, CSF3R, CADPS, CAV2, CAV3, CPT1B, COX7B, CDH13, CD1D, ORAI1, ADGRG6, ADRB1, ASIC5, ACAD11, CFC1, DCXR, ARHGEF4, BCL2L13, BLNK, IBTK, ABCC5, GRB14, GPR182, LIMS2, KCNK5, SLC16A9, EHD3, GPR15, MLIP, MMP2, ITGB4, SLC2A8, LZTR1, NFXL1, OR10R2, NDUFB9, MCEMP1, DES, DYSF,
   h. Tissue protected: Thyroid SLC30A4, TMEM138, ZDHHC6, C2CD2, TMEM100, VIPR2, RMDN2, SLC22A15, SYT13, SLC25A17, SLITRK4, SLC5A5, LPCAT2, PCDHB5, NDUFS8, SLC19A3, ARHGAP5, RETSAT, NEGR1, NUS1, TPO, TRPV6, TMEM40, VPS26B, VHL, WHAMM, SLC25A20, PVR, GPIHBP1, LIN7A, SEL1L3, UPK3BL2, PRICKLE1, MAPKAP1, IFI27, OSBPL7, PSD3, LRRC55, PLPP7, OBSCN, PORCN, SLC7A7, ZC3H12A, QTRT1, SYTL3, TTC7B, FASLG, UPK3BL1, RTP5, SLC22A1, SLC26A4, SLC39A2, SLC39A14, SLCO2B1, SDCBP2, RYK, SH3KBP1, TEX261, REEP4, TMEM171, TCTN3, IYD, HTR7, EHD1, FGFR2, EVA1A, GPR37, KIFC3, EHD4, ABCG1, ABCG2, CAVIN1, CDH6, GBA2, FPR2, RASGRP2, HTR1A, CREB3L2, FAM168B, GPR34, DMTN, FCGR2B, CD99, GPAT2, FGD2, HCN1, FMO3, IPCEF1, GDAP1, CAMK2B, IGSF11, CDH2, FES, ADGRL4, CLIC2, EPHA10, FER1L6, ANKRD13B, ELMO2, AAK1, CAV1, AQP4, ENPEP, CRHR1, CAV2, ANXA1, ORAI1, DCXR, ACKR2, ARHGEF4, EPB41L2, CDH26, BLNK, CDH16, CDH10, AP3B2, CAVIN2, IBTK, MTNR1B, KCNK5, SLC16A9, GPR15, ITGB4, LZTR1, OR10R2, NDUFB9, IKBIP, NCS1,
   i. Tissue protected: Pancreatic endocrine cells (Islets) ZDHHC2, SLC30A4, STXBP1, ACER3, SYT13, SLC25A17, STX1A, RAB3A, PITPNM1, PCDHB5, RTN1, NDUFS8, SCG3, ARHGAP5, RETSAT, NEGR1, POMGNT2, VHL, PTGDR2, SLC25A20, PVR, RAB3B, UCHL1, UPK3BL2, PRICKLE1, SPPL3, MAPKAP1, PTPRN, PTH2R, LRRC55, KIRREL3, OBSCN, NECAB2, SLC17A6, SHISAL2B, SV2A, ZC3H12A, TMEM69, TSPAN7, TTC7B, PLAUR, ZDHHC13, SLC30A8, FASLG, UPK3BL1, SLC22A1, SCAMP5, SYP, SLC39A2, SLCO2B1, SDCBP2, TEX261, MAPK10, SLC35G2, VASN, ATG2B, CRK, EMC4, HTR7, FGFR2, DGCR2, CLTRN, CDH6, GBA2, GNAO1, CREB3L2, FAM169A, FBXO2, GPR34, CASR, CD99, CYP2B6, GDAP1, IGSF11, CDH2, SLC7A8, EPHA10, ELMO2, FXYD2, AMPH, CADPS, CRHR1, CD1D, ASIC5, CFC1, ARHGEF4, EPB41L2, CDH26, BBS7, GAD2, ALOX5, IBTK, ABCC5, GPR153, KIAA2013, OR10R2, NDUFB9, DPP4,
   j. Tissue protected: Pancreatic exocrine cells ZDHHC2, TMEM138, C2CD2, CLEC10A, CD300C, TBC1D3D, TMEM100, RMDN2, SYT13, TMEM97, SLC19A3, SEC16B, RETSAT, PLPP1, POMGNT2, TMEM40, VPS26B, VHL, WHAMM, SLC25A20, PVR, PSCA, KCNH8, SEL1L3, UPK3BL2, TMEM132E, STYK1, SCUBE1, IFI27, PTH2R, NEURL1, LRRC55, PLPP7, OBSCN, NUP54, TMEM72, UGT3A1, VSIG10, ZC3H12A, TTC7B, PLAUR, TBC1D3E, ZDHHC13, TACSTD2, TMPRSS2, FASLG, UPK3BL1, SLC22A1, SYCN, SCAMP5, SLC39A2, SLC39A14, SLCO2B1, SLC4A4, SDCBP2, RYK, SEC11C, SLC19A1, REEP4, TCTN3, CRK, DPEP1, CUZD1, FGFR2, EVA1A, SLC2A12, KIFC3, KIRREL2, FA2H, ABCG1, DSP, ACSL5, CDH6, GBA2, CYSTM1, FPR2, RASGRP2, CREB3L2, FBXO2, GPR34, FCGR2B, CD248, GPAT2, FGD2, CA4, FMO3, GPRC5B, IGSF11, FES, LEPROTL1, FADS3, EPHA10, AQP1, BRI3, KDELR3, CDH9, DGKG, CDC42SE1, ADAM28, ACE2, CNTNAP3, CFTR, FXYD2, CADPS, CAV2, CTSL, C1orf210, CHST4, EPB41L2, PIK3AP1, BBS7, AQP12A, AQP12B, CAVIN2, IBTK, ABCC1, GRB14, SLC16A9, KIAA2013, NFXL1, OR10R2, NDUFB9,

TABLE 3B

List of proteins that meet criteria and have at least medium expression on tissues where immune cell inhibition is desired (Ensembl version 92.38 ID for is provided for each gene symbol)
AXL(ENSG00000167601), VPS52(ENSG00000223501), SCGN(ENSG00000079689), SLC9A2(ENSG00000115616), PCSK5(ENSG00000099139), SELENBP1(ENSG00000143416), SNX24(ENSG00000064652), SMIM24(ENSG00000095932), SMURF1(ENSG00000198742), NAAA(ENSG00000138744), SEC13(ENSG00000157020), SLC41A2(ENSG00000136052), SOCS7(ENSG00000274211), QRFPR(ENSG00000186867), RPH3AL(ENSG00000181031), RAC1(ENSG00000136238), NFAM1(ENSG00000235568), LDLR(ENSG00000130164), GPR65(ENSG00000140030), RAC3(ENSG00000169750), WSCD1(ENSG00000179314), HTRA1(ENSG00000166033), RAB27B(ENSG00000041353), DLC1(ENSG00000164741), PLCB3(ENSG00000149782), MUC1(ENSG00000185499), PSTPIP2(ENSG00000152229), RAB3B(ENSG00000169213),
SDC1(ENSG00000115884), TMEM45A(ENSG00000181458), SPPL3(ENSG00000157837), STYK1(ENSG00000060140), MAPKAP1(ENSG00000119487), IFI27(ENSG00000165949), ITLN1(ENSG00000179914), PTGDR(ENSG00000168229), SGMS1(ENSG00000198964), ITGB6(ENSG00000115221), MUC15(ENSG00000169550), PEX10(ENSG00000157911), OSBPL7(ENSG00000006025), CD46(ENSG00000117335), PROM2(ENSG00000155066), PLPP7(ENSG00000160539), MGAT4A(ENSG00000071073), LRRN2(ENSG00000170382), OBSCN(ENSG00000154358), PROM1(ENSG00000007062), VSIG10(ENSG00000176834), TMEM236(ENSG00000148483), VPS35L(ENSG00000103544), SYTL4(ENSG00000102362), SYTL3(ENSG00000164674), TMEM41A(ENSG00000163900), TSPAN8(ENSG00000127324), UNC13B(ENSG00000198722), SYTL2(ENSG00000137501), TPRA1(ENSG00000163870), OCLN(ENSG00000197822), RNF170(ENSG00000120925), SPHK1(ENSG00000176170), SLC28A3(ENSG00000197506), PTAFR(ENSG00000169403), SYP(ENSG00000102003), PIGZ(ENSG00000119227), PTPRN2(ENSG00000155093), RSAD2(ENSG00000134321), SLC39A14(ENSG00000104635), SLC6A12(ENSG00000111181), SLC6A15(ENSG00000072041), RAB25(ENSG00000132698), SLC20A2(ENSG00000168575), TGFBR1(ENSG00000106799), STK16(ENSG00000115661), TRAF4(ENSG00000076604), SIGLEC14(ENSG00000254415), MTG2(ENSG00000101181), SPTLC3(ENSG00000172296), MARK2(ENSG00000072518), RNF186(ENSG00000178828), TSPAN15(ENSG00000099282), TRAF3IP3(ENSG00000009790), VTCN1(ENSG00000134258), TAS2R38(ENSG00000257138), TMEM39B(ENSG00000121775), CA2(ENSG00000104267), CDHR5(ENSG00000099834), CLDN4(ENSG00000189143), ARL4D(ENSG00000175906), ACVR1B(ENSG00000135503), DPEP1(ENSG00000015413), GSDMB(ENSG00000073605), GPBAR1(ENSG00000179921), SYK(ENSG00000165025), ADCY10(ENSG00000143199), ACSL5(ENSG00000197142), CD177(ENSG00000204936), CLCA1(ENSG00000016490), GLP2R(ENSG00000065325), DSC2(ENSG00000134755), ADGRF1(ENSG00000153292), CLDN5(ENSG00000184113), DSG2(ENSG00000046604), AQP8(ENSG00000103375), CEACAM5(ENSG00000105388), CHST6(ENSG00000183196), CA4(ENSG00000167434), EPCAM(ENSG00000119888), GNG11(ENSG00000127920), CTDNEP1(ENSG00000175826), MAGI1(ENSG00000151276), ENTPD8(ENSG00000188833), FPGS(ENSG00000136877), MAN1C1(ENSG00000117643), FADS2(ENSG00000134824), FER1L6(ENSG00000214814), BEST2(ENSG00000039987), BRI3(ENSG00000164713), CLDN8(ENSG00000156284), GAL3ST2(ENSG00000154252), HTR4(ENSG00000164270), B3GNT8(ENSG00000177191), ELOVL7(ENSG00000164181), B3GALT5(ENSG00000183778), CDHR2(ENSG00000074276), CLDN7(ENSG00000181885), ACE2(ENSG00000130234), CNTNAP3(ENSG00000106714), CDH17(ENSG00000079112), ATP11C(ENSG00000101974), CIB1(ENSG00000185043), ASIC5(ENSG00000256394), DCXR(ENSG00000169738), AKAP10(ENSG00000108599), ARL4C(ENSG00000188042), ANO1(ENSG00000131620), CHST14(ENSG00000169105), ABHD12(ENSG00000100997), C12orf66(ENSG00000174206), ALG14(ENSG00000172339), HTR2B(ENSG00000135914), B3GAT1(ENSG00000109956), HHLA2(ENSG00000114455), MAOA(ENSG00000189221), FGFR4(ENSG00000160867), IBTK(ENSG00000005700), SLC9A3R2(ENSG00000065054), NCEH1(ENSG00000144959), KCNN3(ENSG00000143603), MC4R(ENSG00000166603), MCUB(ENSG00000005059), EPPK1(ENSG00000261150), MSTO1(ENSG00000125459), SLC2A6(ENSG00000160326), KIAA2013(ENSG00000116685), NIPAL1(ENSG00000163293), IL17RA(ENSG00000177663), ITGB4(ENSG00000132470), GPA33(ENSG00000143167), MUC13(ENSG00000173702), OR10R2(ENSG00000198965), PSKH1(ENSG00000159792), NDST2(ENSG00000166507), TGM2(ENSG00000198959), AXL(ENSG00000167601), SLC30A4(ENSG00000104154), SLC22A15(ENSG00000163393), SCEL(ENSG00000136155), AGER(ENSG00000204305), RECK(ENSG00000122707), SCTR(ENSG00000080293), EHD2(ENSG00000024422), PRRG2(ENSG00000126460), SLC39A10(ENSG00000196950), TM4SF1(ENSG00000169908), STEAP1(ENSG00000164647), EHD1(ENSG00000110047), CCR7(ENSG00000126353), CYB5A(ENSG00000166347), AIFM2(ENSG00000042286), LPAR6(ENSG00000139679), AIG1(ENSG00000146416), AQP4(ENSG00000171885), CYB561D1(ENSG00000174151), CACNA2D2(ENSG00000007402), ACE(ENSG00000159640), AQP3(ENSG00000165272), CADM1(ENSG00000182985), LAMP3(ENSG00000078081), EHD3(ENSG00000013016), NSMF(ENSG00000165802), SLC34A2(ENSG00000157765), LRRK2(ENSG00000188906), CLEC10A(ENSG00000132514), PERP(ENSG00000112378), SCEL(ENSG00000136155), SLC41A2(ENSG00000136052), RAC1(ENSG00000136238), RNF145(ENSG00000145860), NFAM1(ENSG00000235568), RAC3(ENSG00000169750), MTMR2(ENSG00000087053), REEP2(ENSG00000132563), TMEM134(ENSG00000172663), SLC10A6(ENSG00000145283), SGMS1(ENSG00000198964), OLFM2(ENSG00000105088), PROM2(ENSG00000155066), KRT1(ENSG00000167768), PLA2G4E(ENSG00000188089), OBSCN(ENSG00000154358), ZP2(ENSG00000103310), SYTL3(ENSG00000164674), TACSTD2(ENSG00000184292), STK16(ENSG00000115661), TRAF3IP3(ENSG00000009790), GJB4(ENSG00000189433), KCNJ8(ENSG00000121361), SYK(ENSG00000165025), FA2H(ENSG00000103089), DSP(ENSG00000096696), CD177(ENSG00000204936), ANO7(ENSG00000146205), CLDN5(ENSG00000184113), EPHA4(ENSG00000116106), CAPNS2(ENSG00000256812), DSC3(ENSG00000134762), GSDMA(ENSG00000167914), FGFBP1(ENSG00000137440), FADS3(ENSG00000221968), FADS2(ENSG00000134824), B3GNT4(ENSG00000176383), DSG1(ENSG00000134760), ADGRF4(ENSG00000153294), ASPRV1(ENSG00000244617), CIB1(ENSG00000185043), DSG3(ENSG00000134757), DCXR(ENSG00000169738), AQP3(ENSG00000165272), CNPPD1(ENSG00000115649), EPPK1(ENSG00000261150), FGFR3(ENSG00000068078), OR10R2(ENSG00000198965), PSKH1(ENSG00000159792), VPS52(ENSG00000223501), MS4A8(ENSG00000166959), MGAT4C(ENSG00000182050), SEC13(ENSG00000157020), TMEM45A(ENSG00000181458), IFI27(ENSG00000165949), ICAM3(ENSG00000076662), PLPP7(ENSG00000160539), PPL(ENSG00000118898), RHCG(ENSG00000140519), TPRA1(ENSG00000163870), RSAD2(ENSG00000134321), S100A8(ENSG00000143546), RAB25(ENSG00000132698), MARK2(ENSG00000072518), CLDN4(ENSG00000189143), DSC2(ENSG00000134755), GPX8(ENSG00000164294), HCN1(ENSG00000164588), CYB561D1(ENSG00000174151), CLEC2A(ENSG00000188393), HTR2B(ENSG00000135914), MAOA(ENSG00000189221), ITGB4(ENSG00000132470), ALOX12(ENSG00000108839), (), CLEC10A(ENSG00000132514), PI4KB(ENSG00000143393), ALPL(ENSG00000162551), ST6GAL1(ENSG00000073849), SLC25A40(ENSG00000075303), SLC27A2(ENSG00000140284), SLCO1B3(ENSG00000111700), SLCO1B1(ENSG00000134538), SLC25A43(ENSG00000077713), SLC27A5(ENSG00000083807), SERINC3(ENSG00000132824), PDZK1(ENSG00000174827), RDH16(ENSG00000139547), SLC25A15(ENSG00000102743), UGT2B4(ENSG00000156096), YIPF1(ENSG00000058799), TFR2(ENSG00000106327), UGT1A6(ENSG00000167165), TNS2(ENSG00000111077), TSPAN2(ENSG00000134198), SLC30A10(ENSG00000196660), GHR(ENSG00000112964), KCNJ8(ENSG00000121361), CYP2D6(ENSG00000100197), FYN(ENSG00000010810), GAS2(ENSG00000148935), SLC2A2(ENSG00000163581), CYP2A7(ENSG00000198077), CYP1A2(ENSG00000140505), CYP3A4(ENSG00000160868), CYP4V2(ENSG00000145476), NINJ1(ENSG00000131669), CYP2C8(ENSG00000138115), DHCR24(ENSG00000116133), CYP2C19(ENSG00000165841), AQP9(ENSG00000103569), ASGR1(ENSG00000141505), ABCB11(ENSG00000073734), ACAD11(ENSG00000240303), ASGR2(ENSG00000161944), CYP3A43(ENSG00000021461), INTS2(ENSG00000108506), CYP2C9(ENSG00000138109), LHFPL2(ENSG00000145685), MFAP3L(ENSG00000198948), ABCC2(ENSG00000023839), DPP4(ENSG00000197635), NPC1L1(ENSG00000015520), SLC45A1(ENSG00000162426), PODXL(ENSG00000128567), TPSG1(ENSG00000116176), PECAM1(ENSG00000261371), SCUBE1(ENSG00000159307), PLA2R1(ENSG00000153246), PNPLA3(ENSG00000100344), MAP6(ENSG00000171533), NPHS2(ENSG00000116218), PTPRO(ENSG00000151490), HYAL2(ENSG00000068001),
CD34(ENSG00000174059), EVI2A(ENSG00000126860), ENG(ENSG00000106991), CR1(ENSG00000203710), FADS3(ENSG00000221968), CRB3(ENSG00000130545), TTYH3(ENSG00000136295), TRPM3(ENSG00000083067), TREH(ENSG00000118094), FCAMR(ENSG00000162897), TM4SF18(ENSG00000163762), SELPLG(ENSG00000110876), SLC5A12(ENSG00000148942), SLC12A1(ENSG00000074803), SLC6A19(ENSG00000174358), SLC22A11(ENSG00000168065), S1PR2(ENSG00000267534), ARHGAP5(ENSG00000100852), RHBG(ENSG00000132677), PLLP(ENSG00000102934), VHL(ENSG00000134086), PARVB(ENSG00000188677), KCNH8(ENSG00000183960), LIN7A(ENSG00000111052), MGAT4B(ENSG00000161013), TMEM132E(ENSG00000181291), OLFM2(ENSG00000105088), NAT8(ENSG00000144035), MPP5(ENSG00000072415), SLC22A6(ENSG00000197901), TMEM72(ENSG00000187783), UGT3A1(ENSG00000145626), ATP6V0A4(ENSG00000105929), SLC7A7(ENSG00000155465), TMEM80(ENSG00000177042), ZP2(ENSG00000103310), TMEM252(ENSG00000181778), TMEM174(ENSG00000164325), RHCG(ENSG00000140519), SLC28A1(ENSG00000156222), SLC22A2(ENSG00000112499), SCAMP5(ENSG00000198794), SLC22A8(ENSG00000149452), RAB11FIP5(ENSG00000135631), SLC6A18(ENSG00000164363), SLC13A2(ENSG00000007216), TMEM213(ENSG00000214128), SYT7(ENSG00000011347), SLC4A4(ENSG00000080493), TMEM178B(ENSG00000261115), PKHD1(ENSG00000170927), SLC35G2(ENSG00000168917), TSPAN16(ENSG00000130167), ENPP6(ENSG00000164303), GGT1(ENSG00000100031), ENPP3(ENSG00000154269), CUBN(ENSG00000107611), GSDMC(ENSG00000147697), CPNE6(ENSG00000100884), CDH6(ENSG00000113361), CALCR(ENSG00000004948), CASR(ENSG00000036828), FOLH1(ENSG00000086205), CNTFR(ENSG00000122756), LRFN4(ENSG00000173621), AQP6(ENSG00000086159), CDC42EP5(ENSG00000167617), SH3BP5(ENSG00000131370), SLC7A9(ENSG00000021488), CLDN10(ENSG00000134873), FXYD2(ENSG00000137731), CMKLR1(ENSG00000174600), CLDN2(ENSG00000165376), ANXA13(ENSG00000104537), CDH16(ENSG00000166589), MOXD1(ENSG00000079931), KCND1(ENSG00000102057), FMO1(ENSG00000010932), LRP2(ENSG00000081479), ALOX12(ENSG00000108839), GRIN2D(ENSG00000105464), NECAP2(ENSG00000157191), MDGA2(ENSG00000139915), SLC27A6(ENSG00000113396), UCP3(ENSG00000175564), CD36(ENSG00000135218), MGAT4C(ENSG00000182050), RASL10B(ENSG00000270885), SLC4A10(ENSG00000144290), RNF145(ENSG00000145860), POPDC2(ENSG00000121577), PLN(ENSG00000198523), SGCG(ENSG00000102683), SRL(ENSG00000185739), DMPK(ENSG00000104936), YRDC(ENSG00000196449), TMEM143(ENSG00000161558), RAB11FIP4(ENSG00000131242), SLCO2B1(ENSG00000137491), TEX261(ENSG00000144043), SCN7A(ENSG00000136546), DSP(ENSG00000096696), CDH15(ENSG00000129910), GNB5(ENSG00000069966), RASGRP2(ENSG00000068831), CAVIN4(ENSG00000170681), CHRNA1(ENSG00000138435), CD248(ENSG00000174807), GPAT2(ENSG00000186281), LEPROTL1(ENSG00000104660), CDC42SE1(ENSG00000197622), ADAM28(ENSG00000042980), CSF3R(ENSG00000119535), CDH13(ENSG00000140945), ADGRG6(ENSG00000112414), ADRB1(ENSG00000043591), AP4E1(ENSG00000081014), CNPPD1(ENSG00000115649), MYZAP(ENSG00000263155), GPR182(ENSG00000166856), GPR15(ENSG00000154165), SLC16A10(ENSG00000112394), PRKCA(ENSG00000154229), DES(ENSG00000175084), TMEM100(ENSG00000166292), SLC5A5(ENSG00000105641), MPP7(ENSG00000150054), TPO(ENSG00000115705), PSD3(ENSG00000156011), IYD(ENSG00000009765), DUOX1(ENSG00000137857), EPHA4(ENSG00000116106), HCN1(ENSG00000164588), IPCEF1(ENSG00000074706), AAK1(ENSG00000115977), LRP1B(ENSG00000168702), STX1A(ENSG00000106089), RTN1(ENSG00000139970), SCG3(ENSG00000104112), PTGDR2(ENSG00000183134), PTPRN(ENSG00000054356), NECAB2(ENSG00000103154), SLC17A6(ENSG00000091664), SV2A(ENSG00000159164), TSPAN7(ENSG00000156298), SLC30A8(ENSG00000164756), DGCR2(ENSG00000070413), GNAO1(ENSG00000087258), FAM169A(ENSG00000198780), ATP9A(ENSG00000054793), CLU(ENSG00000120885), ATP1A2(ENSG00000018625), GAD2(ENSG00000136750), ALOX5(ENSG00000012779), TMEM97(ENSG00000109084), SYCN(ENSG00000179751), CUZD1(ENSG00000138161), GP2(ENSG00000169347), KIRREL2(ENSG00000126259), FA2H(ENSG00000103089), CFTR(ENSG00000001626), PIK3AP1(ENSG00000155629), AQP12A(ENSG00000184945), AQP12B(ENSG00000185176), UGT2A3(ENSG00000135220), CORO7(ENSG00000262246), GABARAPL2(ENSG00000034713), SPCS1(ENSG00000114902), TNFSF12(ENSG00000239697), PLEKHA3(ENSG00000116095), TVP23B(ENSG00000171928), WHAMM(ENSG00000156232), SLCO4A1(ENSG00000101187), SLC9A3(ENSG00000066230), PCDH11X(ENSG00000102290), PALM(ENSG00000099864), VEPH1(ENSG00000197415), CNEP1R1(ENSG00000205423), GOLGA2(ENSG00000167110), AATK(ENSG00000181409), SLC26A3(ENSG00000091138), UGT2B17(ENSG00000197888), SERAC1(ENSG00000122335), TVP23C(ENSG00000175106), UGT2B28(ENSG00000135226), TMEM253(ENSG00000232070), UGT2B15(ENSG00000196620), ARHGAP17(ENSG00000140750), C2orf88(ENSG00000187699), GOPC(ENSG00000047932), SNX6(ENSG00000129515), PHACTR2(ENSG00000112419), NAPG(ENSG00000134265), NAPB(ENSG00000125814), TMEM171(ENSG00000157111), TMEM30A(ENSG00000112697), CLSTN1(ENSG00000171603), CEACAM1(ENSG00000079385), FPR2(ENSG00000171049), FKBP2(ENSG00000173486), CHDH(ENSG00000016391), FAM126B(ENSG00000155744), HTR1A(ENSG00000178394), GLT8D2(ENSG00000120820), GATM(ENSG00000171766), FLNC(ENSG00000128591), DIAPH1(ENSG00000131504), CA12(ENSG00000074410), DPP10(ENSG00000175497), DGKQ(ENSG00000145214), GBP2(ENSG00000162645), LRRC37B(ENSG00000185158), AP3M1(ENSG00000185009), ANKRD27(ENSG00000105186), ANO8(ENSG00000074855), ARRB1(ENSG00000137486), C5AR2(ENSG00000134830), EBP(ENSG00000147155), HSD3B7(ENSG00000099377), SMPD4(ENSG00000136699), SLC16A6(ENSG00000108932), GFRA2(ENSG00000168546), GNG12(ENSG00000172380), GLRA2(ENSG00000101958), NIPA2(ENSG00000140157), DPY19L1(ENSG00000173852), RPS6KC1(ENSG00000136643), TRIM13(ENSG00000204977), PLSCR4(ENSG00000114698), SLC25A17(ENSG00000100372), PTPRG(ENSG00000144724), SGIP1(ENSG00000118473), PSD4(ENSG00000125637), PHLPP2(ENSG00000040199), EHD4(ENSG00000103966), SLC44A2(ENSG00000129353), AOC3(ENSG00000131471), ELMO2(ENSG00000062598), LDLRAD2(ENSG00000187942), PI4KA(ENSG00000241973), CACNB2(ENSG00000165995), TGFBR3(ENSG00000069702), PALM(ENSG00000099864), VEPH1(ENSG00000197415), GOLGA2(ENSG00000167110), AATK(ENSG00000181409), TRPM2(ENSG00000142185), RMC1(ENSG00000141452), SERAC1(ENSG00000122335), C2orf88(ENSG00000187699), GOPC(ENSG00000047932), RAB29(ENSG00000117280), TMEM171(ENSG00000157111), FAM126B(ENSG00000155744), GATM(ENSG00000171766), CD99(ENSG00000002586), CA12(ENSG00000074410), GBP2(ENSG00000162645), AP3M1(ENSG00000185009), ARRB1(ENSG00000137486), RPS6KC1(ENSG00000136643), CACNB2(ENSG00000165995), TNFSF12(ENSG00000239697), TYR(ENSG00000077498), DCT(ENSG00000080166), ZC4H2(ENSG00000126970), RYK(ENSG00000163785), PMEL(ENSG00000185664), CYB561A3(ENSG00000162144), FAM210A(ENSG00000177150), MALL(ENSG00000144063), BST1(ENSG00000109743), HSD3B7(ENSG00000099377), SYT13(ENSG00000019505), SVEP1(ENSG00000165124), KCNQ4(ENSG00000117013), SORD(ENSG00000140263), MME(ENSG00000196549), SPRED1(ENSG00000166068), TBCD(ENSG00000141556), FCER1A(ENSG00000179639), GCHFR(ENSG00000137880), AADAC(ENSG00000114771), FAM210A(ENSG00000177150), FMO3(ENSG00000007933), ABCB4(ENSG00000005471), FES(ENSG00000182511), KMO(ENSG00000117009), BCL2L10(ENSG00000137875), UGCG(ENSG00000148154), ENPEP(ENSG00000138792), CTSL(ENSG00000135047), HSD11B1(ENSG00000117594), IGFLR1(ENSG00000126246), SLC10A1(ENSG00000100652), ZAP70(ENSG00000115085), KDR(ENSG00000128052), MAPT(ENSG00000186868), TGFBR3(ENSG00000069702), KIRREL1(ENSG00000183853),
KSR2(ENSG00000171435), MRGPRF(ENSG00000172935), PTH2R(ENSG00000144407), PRKAR2B(ENSG00000005249), RMC1(ENSG00000141452), SNCA(ENSG00000145335), RYK(ENSG00000163785), SLC35B1(ENSG00000121073), CD99(ENSG00000002586), AQP1(ENSG00000240583), LIMS2(ENSG00000072163), ITGA8(ENSG00000077943), VNN1(ENSG00000112299), ABCD4(ENSG00000119688), UBE2J1(ENSG00000198833), SLC5A2(ENSG00000140675), SLC34A3(ENSG00000198569), NEGR1(ENSG00000172260), PTH1R(ENSG00000160801), PTPRD(ENSG00000153707), MPP6(ENSG00000105926), SLC34A1(ENSG00000131183), PRICKLE1(ENSG00000139174), KIRREL3(ENSG00000149571), XPNPEP2(ENSG00000122121), VAC14(ENSG00000103043), QTRT1(ENSG00000213339), TSPAN13(ENSG00000106537), TTC7B(ENSG00000165914), SLC13A3(ENSG00000158296), RAB29(ENSG00000117280), PEAR1(ENSG00000187800), PRRT3(ENSG00000163704), OPRD1(ENSG00000116329), ADCY7(ENSG00000121281), FAM151A(ENSG00000162391), CREB3L2(ENSG00000182158), TM7SF2(ENSG00000149809), L1CAM(ENSG00000198910), BSND(ENSG00000162399), MPP1(ENSG00000130830), COX7B(ENSG00000131174), AP1S3(ENSG00000152056), EPB41L3(ENSG00000082397), ABCC5(ENSG00000114770), SLC43A2(ENSG00000167703), NDUFS1(ENSG00000023228), GAL3ST1(ENSG00000128242), CHL1(ENSG00000134121), TCTN2(ENSG00000168778), CAP2(ENSG00000112186), TLN2(ENSG00000171914), SGCB(ENSG00000163069), BVES(ENSG00000112276), P2RX6(ENSG00000099957), P2RY6(ENSG00000171631), LANCL2(ENSG00000132434), SIPA1(ENSG00000213445), TBC1D3(ENSG00000274611), SLC25A25(ENSG00000148339), CNTN3(ENSG00000113805), CKMT2(ENSG00000131730), CACNA2D1(ENSG00000153956), FNDC5(ENSG00000160097), SLC2A4(ENSG00000181856), HHATL(ENSG00000010282), CAMK2B(ENSG00000058404), ABHD16A(ENSG00000204427), DMD(ENSG00000198947), CPT1B(ENSG00000205560), AKAP6(ENSG00000151320), NCAM1(ENSG00000149294), MLIP(ENSG00000146147), VPS33B(ENSG00000184056), SCNN1D(ENSG00000162572), NUS1(ENSG00000153989), ZC4H2(ENSG00000126970), RNFT2(ENSG00000135119), MALL(ENSG00000144063), ARRDC1(ENSG00000197070), MTNR1B(ENSG00000134640), FAM234B(ENSG00000084444), SHISAL2B(ENSG00000145642), CD82(ENSG00000085117), CADPS(ENSG00000163618), STIMATE(ENSG00000213533), MED28(ENSG00000118579), NEURL1(ENSG00000107954), CDC42EP1(ENSG00000128283), TJAP1(ENSG00000137221), TMEM138(ENSG00000149483), TMEM237(ENSG00000155755), SLC7A6(ENSG00000103064), ATP8B2(ENSG00000143515), SLC46A1(ENSG00000076351), ST3GAL6(ENSG00000064225), LPCAT2(ENSG00000087253), SLC6A6(ENSG00000131389), PEX1(ENSG00000127980), PEMT(ENSG00000133027), NPDC1(ENSG00000107281), NTRK1(ENSG00000198400), GOLGA8B(ENSG00000215252), GRIK2(ENSG00000164418), PCDHGA10(ENSG00000253846), HRAS(ENSG00000174775), PILRA(ENSG00000085514), PKHD1L1(ENSG00000205038), USP8(ENSG00000138592), YIF1B(ENSG00000167645), RHBDL2(ENSG00000158315), SLC25A20(ENSG00000178537), PLA2G2A(ENSG00000188257), KRAS(ENSG00000133703), STXBP6(ENSG00000168952), NRAS(ENSG00000213281), TENM3(ENSG00000218336), STXBP3(ENSG00000116266), TAOK2(ENSG00000149930), MITD1(ENSG00000158411), PRSS8(ENSG00000052344), GNPNAT1(ENSG00000100522), LRRC55(ENSG00000183908), NUTF2(ENSG00000102898), KCNQ1(ENSG00000053918), NECTIN1(ENSG00000110400), NALCN(ENSG00000102452), SLC51B(ENSG00000186198), SLC43A3(ENSG00000134802), SELENOT(ENSG00000198843), SLC9A1(ENSG00000090020), ST7(ENSG00000004866), SNTA1(ENSG00000101400), TUB(ENSG00000166402), TEX264(ENSG00000164081), ZNRF1(ENSG00000186187), TMEM69(ENSG00000159596), ZFYVE9(ENSG00000157077), SLC35A3(ENSG00000117620), PCDH1(ENSG00000156453), RNF125(ENSG00000101695), QPCTL(ENSG00000011478), SLC45A4(ENSG00000022567), RAB27A(ENSG00000069974), SLC12A6(ENSG00000140199), PARD3B(ENSG00000116117), SMIM6(ENSG00000259120), MIA3(ENSG00000154305), STK17B(ENSG00000081320), SSTR1(ENSG00000139874), TMEM204(ENSG00000131634), SNX18(ENSG00000178996), PIK3R5(ENSG00000141506), RHOU(ENSG00000116574), SLC4A5(ENSG00000188687), MSMO1(ENSG00000052802), TMIGD2(ENSG00000167664), TYRO3(ENSG00000092445), PRKCZ(ENSG00000067606), EMC9(ENSG00000100908), FAM83B(ENSG00000168143), FKRP(ENSG00000181027), FMO5(ENSG00000131781), EMC6(ENSG00000127774), KCTD3(ENSG00000136636), GOLPH3(ENSG00000113384), ATP2C2(ENSG00000064270), ABCG2(ENSG00000118777), EPHB3(ENSG00000182580), DGKE(ENSG00000153933), CYP4F11(ENSG00000171903), GBF1(ENSG00000107862), DMTN(ENSG00000158856), GOLGA8A(ENSG00000175265), WDPCP(ENSG00000143951), CNR1(ENSG00000118432), ARFGEF3(ENSG00000112379), CARMIL2(ENSG00000159753), CHMP5(ENSG00000086065), KPNA2(ENSG00000182481), GPR82(ENSG00000171657), IGSF11(ENSG00000144847), IL17RC(ENSG00000163702), EVA1B(ENSG00000142694), LPP(ENSG00000145012), EPHA10(ENSG00000183317), FERMT1(ENSG00000101311), ABCA10(ENSG00000154263), DTX3L(ENSG00000163840), CYB561(ENSG00000008283), AVEN(ENSG00000169857), CXADR(ENSG00000154639), GRAMD1A(ENSG00000089351), B3GNT7(ENSG00000156966), CHMP2B(ENSG00000083937), ALDH3A2(ENSG00000072210), NISCH(ENSG00000010322), GIPC1(ENSG00000123159), FUT3(ENSG00000171124), GLUL(ENSG00000135821), IL17RE(ENSG00000163701), MARCKSL1(ENSG00000175130), EFHD2(ENSG00000142634), FUT6(ENSG00000156413), GPER1(ENSG00000164850), HCN3(ENSG00000143630), MBTPS1(ENSG00000140943), JAML(ENSG00000160593), GPC1(ENSG00000063660), LZTR1(ENSG00000099949), NEU4(ENSG00000204099), MUC4(ENSG00000145113), NAPEPLD(ENSG00000161048), IL4R(ENSG00000077238), STX5(ENSG00000162236), S100A9(ENSG00000163220), ARHGEF1(ENSG00000076928), GPC5(ENSG00000179399), DCBLD1(ENSG00000164465), CLIC2(ENSG00000155962), TMEM243(ENSG00000135185), TJAP1(ENSG00000137221), TMEM138(ENSG00000149483), TMEM237(ENSG00000155755), RXFP1(ENSG00000171509), SLC5A6(ENSG00000138074), SLC6A6(ENSG00000131389), GRIK2(ENSG00000164418), TMEM40(ENSG00000088726), LYPD3(ENSG00000124466), SLC8A1(ENSG00000183023), SLC43A3(ENSG00000134802), SLC9A1(ENSG00000090020), SNTA1(ENSG00000101400), SRC(ENSG00000197122), TUB(ENSG00000166402), ZNRF1(ENSG00000186187), TMEM69(ENSG00000159596), THBD(ENSG00000178726), SLC35A3(ENSG00000117620), RNF125(ENSG00000101695), TMTC4(ENSG00000125247), QPCTL(ENSG00000011478), PARD3B(ENSG00000116117), SNX18(ENSG00000178996), LY6D(ENSG00000167656), S100A9(ENSG00000163220), MSMO1(ENSG00000052802), EMC9(ENSG00000100908), FAM83B(ENSG00000168143), FRS3(ENSG00000137218), EPHB3(ENSG00000182580), DMTN(ENSG00000158856), ARFGEF3(ENSG00000112379), KPNA2(ENSG00000182481), EVA1B(ENSG00000142694), LPP(ENSG00000145012), EPHA10(ENSG00000183317), FERMT1(ENSG00000101311), ABCA10(ENSG00000154263), CDH5(ENSG00000179776), AVEN(ENSG00000169857), CXADR(ENSG00000154639), ORAI1(ENSG00000276045), ALDH3A2(ENSG00000072210), NISCH(ENSG00000010322), MARCKSL1(ENSG00000175130), GPER1(ENSG00000164850), TACR1(ENSG00000115353), GPC1(ENSG00000063660), TMEM243(ENSG00000135185), NTRK1(ENSG00000198400), GOLGA8B(ENSG00000215252), PKHD1L1(ENSG00000205038), STXBP3(ENSG00000116266), LRRC55(ENSG00000183908), KCNQ1(ENSG00000053918), NECTIN1(ENSG00000110400), ST7(ENSG00000004866), SMIM6(ENSG00000259120), RHOU(ENSG00000116574), TYRO3(ENSG00000092445), KCTD3(ENSG00000136636), ATP2C2(ENSG00000064270), GPC5(ENSG00000179399), GBF1(ENSG00000107862), FAT2(ENSG00000086570), GOLGA8A(ENSG00000175265), CARMIL2(ENSG00000159753), FAM241B(ENSG00000171224), IL17RC(ENSG00000163702), GIPC1(ENSG00000123159), TECR(ENSG00000099797), TMCC1(ENSG00000172765), SLC8A1(ENSG00000183023), RFTN1(ENSG00000131378), TM4SF4(ENSG00000169903), CYP2W1(ENSG00000073067), CACNA1A(ENSG00000141837), CYP4F3(ENSG00000186529),
EPHX1(ENSG00000143819), CYP2A6(ENSG00000255974), ATP5F1C(ENSG00000165629), CYP4F2(ENSG00000186115), CDH5(ENSG00000179776), CYP2A13(ENSG00000197838), CYP2E1(ENSG00000130649), EPB41L5(ENSG00000115109), KCNJ11(ENSG00000187486), MTCH2(ENSG00000109919), PDGFRB(ENSG00000113721), TRPV6(ENSG00000165125), MCAM(ENSG00000076706), NEU3(ENSG00000162139), KCNAB1(ENSG00000169282), LDLRAD3(ENSG00000179241), SMIM18(ENSG00000253457), SORT1(ENSG00000134243), ZBED3(ENSG00000132846), FAM241B(ENSG00000171224), GPRC5B(ENSG00000167191), GPR153(ENSG00000158292), NFXL1(ENSG00000170448), KIAA0040(ENSG00000235750), STRA6(ENSG00000137868), RGS16(ENSG00000143333), VPS26B(ENSG00000151502), RASA4(ENSG00000105808), SRC(ENSG00000197122), SLC52A3(ENSG00000101276), TMEM184A(ENSG00000164855), SNORC(ENSG00000182600), SLC23A1(ENSG00000170482), TMEM242(ENSG00000215712), GJA5(ENSG00000265107), SLC7A8(ENSG00000092068), SLC25A31(ENSG00000151475), ATP12A(ENSG00000075673), NXPE2(ENSG00000204361), SLC14A1(ENSG00000141469), TMEM106B(ENSG00000106460), SUSD3(ENSG00000157303), PITPNM1(ENSG00000110697), RYR2(ENSG00000198626), TMEM119(ENSG00000183160), PPM1L(ENSG00000163590), FAM168B(ENSG00000152102), ATP1A3(ENSG00000105409), ATP2A2(ENSG00000174437), CAV3(ENSG00000182533), ORAI1(ENSG00000276045), KIAA1109(ENSG00000138688), RHBDD2(ENSG00000005486), TMTC4(ENSG00000125247), GDAP1(ENSG00000104381), FZD5(ENSG00000163251), CD300A(ENSG00000167851), STXBP1(ENSG00000136854), GCSAM(ENSG00000174500), DGKZ(ENSG00000149091), SELENOK(ENSG00000113811), ATP11B(ENSG00000058063), KDELR3(ENSG00000100196), MYOC(ENSG00000034971), TMEM60(ENSG00000135211), SLC47A1(ENSG00000142494), SH3BP4(ENSG00000130147), SLC19A3(ENSG00000135917), RNF144B(ENSG00000137393), SYBU(ENSG00000147642), MYORG(ENSG00000164976), MCOLN1(ENSG00000090674), SLC16A14(ENSG00000163053), RAB43(ENSG00000172780), TMEM219(ENSG00000149932), CRK(ENSG00000167193), BMPR1A(ENSG00000107779), KCNMA1(ENSG00000156113), ABCG8(ENSG00000143921), BCL2(ENSG00000171791), ANPEP(ENSG00000166825), NIPAL4(ENSG00000172548), HPS6(ENSG00000166189), ILK(ENSG00000166333), IL10RB(ENSG00000243646), MXRA7(ENSG00000182534), ENPP1(ENSG00000197594), F2R(ENSG00000181104), MCOLN1(ENSG00000090674), SLC16A14(ENSG00000163053), CRK(ENSG00000167193), ATRAID(ENSG00000138085), ABCG8(ENSG00000143921), SH3BP4(ENSG00000130147), ARHGAP21(ENSG00000107863), TMEM219(ENSG00000149932), BCL2(ENSG00000171791), BBS1(ENSG00000174483), APLNR(ENSG00000134817), RAB17(ENSG00000124839), ENPP1(ENSG00000197594), PEX11G(ENSG00000104883), AKR1A1(ENSG00000117448), CYP2B6(ENSG00000197408), AP1S2(ENSG00000182287), BBS1(ENSG00000174483), ASPHD1(ENSG00000174939), F2R(ENSG00000181104), SREBF1(ENSG00000072310), VASN(ENSG00000168140), AIF1L(ENSG00000126878), ABCC1(ENSG00000103222), DYSF(ENSG00000135636), MGAM(ENSG00000257335), TMEM52B(ENSG00000165685), ATP6V1D(ENSG00000100554), UST(ENSG00000111962), RUFY3(ENSG00000018189), ARHGAP21(ENSG00000107863), AP4M1(ENSG00000221838), ATRAID(ENSG00000138085), CLIC4(ENSG00000169504), ANK2(ENSG00000145362), ST8SIA2(ENSG00000140557), PAM(ENSG00000145730), SLC26A4(ENSG00000091137), CSK(ENSG00000103653), CYP4X1(ENSG00000186377), ZDHHC2(ENSG00000104219), SLC30A7(ENSG00000162695), SFT2D2(ENSG00000213064), TMEM74B(ENSG00000125895), TNFSF14(ENSG00000125735), TTC17(ENSG00000052841), ACY3(ENSG00000132744), CHST11(ENSG00000171310), FIBCD1(ENSG00000130720), ZGRF1(ENSG00000138658), SRI(ENSG00000075142), SLC26A2(ENSG00000155850), SFXN5(ENSG00000144040), NDUFS8(ENSG00000110717), PARD3(ENSG00000148498), PLPP1(ENSG00000067113), RHOH(ENSG00000168421), PIGR(ENSG00000162896), TPCN1(ENSG00000186815), TULP3(ENSG00000078246), TJP3(ENSG00000105289), TCIRG1(ENSG00000110719), PIGU(ENSG00000101464), RAP1GAP(ENSG00000076864), MFSD10(ENSG00000109736), SEL1L3(ENSG00000091490), SERP1(ENSG00000120742), SCARA3(ENSG00000168077), NECAPl(ENSG00000089818), PLIN2(ENSG00000147872), ABCC3(ENSG00000108846), GPR183(ENSG00000169508), SLC24A3(ENSG00000185052), MACF1(ENSG00000127603), MGST2(ENSG00000085871), DENND5B(ENSG00000170456), MEGF8(ENSG00000105429), PNKD(ENSG00000127838), SERP2(ENSG00000151778), TNFAIP8L3(ENSG00000183578), SLC3A1(ENSG00000138079), VPS28(ENSG00000160948), TMEM64(ENSG00000180694), TSPAN31(ENSG00000135452), TNFRSF14(ENSG00000157873), TMEM201(ENSG00000188807), MCUR1(ENSG00000050393), SUSD6(ENSG00000100647), RAB31(ENSG00000168461), TRPM4(ENSG00000130529), VTI1B(ENSG00000100568), TNFRSF10A(ENSG00000104689), TMEM164(ENSG00000157600), STK38L(ENSG00000211455), IMMT(ENSG00000132305), ADCK2(ENSG00000133597), ATG2B(ENSG00000066739), CCR10(ENSG00000184451), GPC4(ENSG00000076716), CX3CR1(ENSG00000168329), DPY19L4(ENSG00000156162), SLC48A1(ENSG00000211584), CLEC16A(ENSG00000038532), FCER1G(ENSG00000158869), DMXL2(ENSG00000104093), FAR2(ENSG00000064763), CLN6(ENSG00000128973), CTNND1(ENSG00000198561), FXYD6(ENSG00000137726), ARL8B(ENSG00000134108), ELOVL1(ENSG00000066322), SLC37A4(ENSG00000137700), HRH1(ENSG00000196639), GJA4(ENSG00000187513), GALNT16(ENSG00000100626), DENND5A(ENSG00000184014), ALG2(ENSG00000119523), CLDND1(ENSG00000080822), CYP20A1(ENSG00000119004), B3GNT6(ENSG00000198488), TMEM30B(ENSG00000182107), AGPS(ENSG00000018510), CASD1(ENSG00000127995), ABCD3(ENSG00000117528), MPZL1(ENSG00000197965), NBAS(ENSG00000151779), SLC2A10(ENSG00000197496), APOOL(ENSG00000155008), HIGD1A(ENSG00000181061), MT-ND4(ENSG00000198886), MFSD2A(ENSG00000168389), PDLIM4(ENSG00000131435), RNF144A(ENSG00000151692), PIGS(ENSG00000087111), CTXN2(ENSG00000233932), AP3S1(ENSG00000177879), CD63(ENSG00000135404), AP3S2(ENSG00000157823), CACNA1C(ENSG00000151067), CAVIN2(ENSG00000168497), ZDHHC2(ENSG00000104219), ACER3(ENSG00000078124), CHST11(ENSG00000171310), TULP3(ENSG00000078246), ITGA9(ENSG00000144668), PLIN2(ENSG00000147872), ABCC3(ENSG00000108846), GPR183(ENSG00000169508), MGST2(ENSG00000085871), PNKD(ENSG00000127838), TMEM260(ENSG00000070269), UBR4(ENSG00000127481), VTI1B(ENSG00000100568), TMEM164(ENSG00000157600), DMXL2(ENSG00000104093), ARL8A(ENSG00000143862), TESC(ENSG00000088992), CTNND1(ENSG00000198561), ARL8B(ENSG00000134108), ELOVL1(ENSG00000066322), ANKRD13A(ENSG00000076513), GALNT16(ENSG00000100626), COL13A1(ENSG00000197467), AGPS(ENSG00000018510), ABCD3(ENSG00000117528), MPZL1(ENSG00000197965), NBAS(ENSG00000151779), APOOL(ENSG00000155008), MFSD2A(ENSG00000168389), TCIRG1(ENSG00000110719), RAP1GAP(ENSG00000076864), SEL1L3(ENSG00000091490), MERTK(ENSG00000153208), VPS28(ENSG00000160948), MCUR1(ENSG00000050393), B3GNT5(ENSG00000176597), FAR2(ENSG00000064763), SLC37A4(ENSG00000137700), SLC2A11(ENSG00000133460), MERTK(ENSG00000153208), TMEM260(ENSG00000070269), ZDHHC8(ENSG00000099904), UBR4(ENSG00000127481), TSC2(ENSG00000103197), SERINC1(ENSG00000111897), EVA1A(ENSG00000115363), B3GNT5(ENSG00000176597), TESC(ENSG00000088992), CDH2(ENSG00000170558), ABCA8(ENSG00000141338), SLC31A1(ENSG00000136868), CMTM8(ENSG00000170293), CYTH2(ENSG00000105443), GRB14(ENSG00000115290), GK(ENSG00000198814), POMGNT1(ENSG00000085998), ACER3(ENSG00000078124), RDX(ENSG00000137710), PDGFC(ENSG00000145431), NOXA1(ENSG00000188747), UCHL1(ENSG00000154277), ITGA9(ENSG00000144668), PLCD4(ENSG00000115556), TMPRSS2(ENSG00000184012), SLC25A23(ENSG00000125648), JAM3(ENSG00000166086), ANKRD13A(ENSG00000076513), ACKR3(ENSG00000144476), TMEM63B(ENSG00000137216), HAS3(ENSG00000103044), KCNK5(ENSG00000164626), SLC2A11(ENSG00000133460),
NTRK3(ENSG00000140538), PTPRB(ENSG00000127329), BCL2L13(ENSG00000099968), ICA1(ENSG00000003147), GPR63(ENSG00000112218), AHCYL1(ENSG00000168710), LMAN2(ENSG00000169223), ARL8A(ENSG00000143862), CLCN5(ENSG00000171365), ENO2(ENSG00000111674), CHRM3(ENSG00000133019), TMEM128(ENSG00000132406), COL13A1(ENSG00000197467), ARFGAP3(ENSG00000242247), SLC16A7(ENSG00000118596), TMEM263(ENSG00000151135), SEMA5A(ENSG00000112902), SLC26A1(ENSG00000145217), ADIPOR2(ENSG00000006831), SLC39A2(ENSG00000165794), EFHD1(ENSG00000115468), ABCG1(ENSG00000160179), ADGRA3(ENSG00000152990), ANKFY1(ENSG00000185722), BIK(ENSG00000100290), VIPR2(ENSG00000106018), TMEM43(ENSG00000170876), CAVIN1(ENSG00000177469), CDH11(ENSG00000140937), FCAR(ENSG00000186431), CAV1(ENSG00000105974), ANXA1(ENSG00000135046), VIPR2(ENSG00000106018), TMEM263(ENSG00000151135), SEMA5A(ENSG00000112902), TMEM43(ENSG00000170876), CLCA2(ENSG00000137975), CAVIN1(ENSG00000177469), ADGRA3(ENSG00000152990), GJA1(ENSG00000152661), CAV1(ENSG00000105974), ANXA1(ENSG00000135046), SLC26A1(ENSG00000145217), ADIPOR2(ENSG00000006831), GAA(ENSG00000171298), ZDHHC6(ENSG00000023041), ITGB1(ENSG00000150093), GJA1(ENSG00000152661), COL6A2(ENSG00000142173), KCNJ1(ENSG00000151704), UPK3BL2(ENSG00000284981), TMEM255B(ENSG00000184497), UPK3BL1(ENSG00000267368), ABHD17C(ENSG00000136379), TRPV4(ENSG00000111199), CFC1(ENSG00000136698), RNF215(ENSG00000099999), MAST2(ENSG00000086015), VSTM5(ENSG00000214376), C2CD2L(ENSG00000172375), PHKA1(ENSG00000067177), IFFO1(ENSG00000010295), MARCKS(ENSG00000277443), SLC6A14(ENSG00000268104), RNF103(ENSG00000239305), C2CD2L(ENSG00000172375), IGSF8(ENSG00000162729), PHKA1(ENSG00000067177), MARCKS(ENSG00000277443), SLC6A14(ENSG00000268104), SLC25A33(ENSG00000171612), XKR9(ENSG00000221947), SLC25A33(ENSG00000171612), AKAP12(ENSG00000131016), XKR9(ENSG00000221947), PPP1R3A(ENSG00000154415), TMEM245(ENSG00000106771), TEX2(ENSG00000136478), SCD5(ENSG00000145284), RRAD(ENSG00000166592), SNX1(ENSG00000028528), SMPD2(ENSG00000135587), MSRA(ENSG00000175806), MFN2(ENSG00000116688), UTRN(ENSG00000152818), ARHGEF12(ENSG00000196914), FGFRL1(ENSG00000127418), GABARAPL1(ENSG00000139112), FMNL3(ENSG00000161791), CLN5(ENSG00000102805), GNAI3(ENSG00000065135), GNAI1(ENSG00000127955), DAPP1(ENSG00000070190), ICAM1(ENSG00000090339), SNX1(ENSG00000028528), GNAI3(ENSG00000065135), ARHGEF12(ENSG00000196914), GNAI1(ENSG00000127955), FMNL3(ENSG00000161791), CLN5(ENSG00000102805), CLEC2B(ENSG00000110852), NDUFAF5(ENSG00000101247), CLEC2B(ENSG00000110852), LRIG1(ENSG00000144749), TSPAN33(ENSG00000158457), MRC2(ENSG00000011028), ARMCX2(ENSG00000184867), DAG1(ENSG00000173402), LRRC24(ENSG00000254402), TMED6(ENSG00000157315), SCN4B(ENSG00000177098), DST(ENSG00000151914), SLC22A16(ENSG00000004809), SPIRE1(ENSG00000134278), SIRPB1(ENSG00000101307), PVR(ENSG00000073008), SLC25A36(ENSG00000114120), PCDHB15(ENSG00000113248), SNX7(ENSG00000162627), SLC11A1(ENSG00000018280), MPC2(ENSG00000143158), MGLL(ENSG00000074416), ITPR2(ENSG00000123104), MFSD11(ENSG00000092931), RINT1(ENSG00000135249), TMEM86A(ENSG00000151117), SLC38A5(ENSG00000017483), SUSD1(ENSG00000106868), REEP4(ENSG00000168476), KCNMB3(ENSG00000171121), HHAT(ENSG00000054392), CDK14(ENSG00000058091), APOBR(ENSG00000184730), ADCK5(ENSG00000173137), TMEM37(ENSG00000171227), ATP8B1(ENSG00000081923), LFNG(ENSG00000106003), MRAP(ENSG00000170262), NDUFB9(ENSG00000147684), PAG1(ENSG00000076641), VAT1(ENSG00000108828), SPIRE1(ENSG00000134278), VAT1(ENSG00000108828), MGLL(ENSG00000074416), TMEM86A(ENSG00000151117), APOBR(ENSG00000184730), ATP8B1(ENSG00000081923), SCN4B(ENSG00000177098), SLC11A1(ENSG00000018280), MPC2(ENSG00000143158), ITPR2(ENSG00000123104), REEP4(ENSG00000168476), CCND1(ENSG00000110092), SLC38A9(ENSG00000177058), KCNJ16(ENSG00000153822), NPHS1(ENSG00000161270), GREB1(ENSG00000196208), DAB2(ENSG00000153071), ACVRL1(ENSG00000139567), ATG9A(ENSG00000198925), FXYD7(ENSG00000221946), FKBP11(ENSG00000134285), TSPAN12(ENSG00000106025), TBC1D9B(ENSG00000197226), TSPAN3(ENSG00000140391), SNX4(ENSG00000114520), SMAGP(ENSG00000170545), C2CD2(ENSG00000157617), PLPP2(ENSG00000141934), TREM2(ENSG00000095970), TMEM94(ENSG00000177728), PLXNA1(ENSG00000114554), SLC25A46(ENSG00000164209), RNF5(ENSG00000204308), NAGPA(ENSG00000103174), NBEA(ENSG00000172915), TFPI(ENSG00000003436), RAB20(ENSG00000139832), RASD2(ENSG00000100302), SPCS3(ENSG00000129128), MGAT1(ENSG00000131446), KCNK1(ENSG00000135750), POMT2(ENSG00000009830), HEG1(ENSG00000173706), ILDR1(ENSG00000145103), LYPLA1(ENSG00000120992), TMEM159(ENSG00000011638), NUCB2(ENSG00000070081), VPS11(ENSG00000160695), SIGIRR(ENSG00000185187), TMEM53(ENSG00000126106), SLC19A2(ENSG00000117479), TECPR1(ENSG00000205356), VPS41(ENSG00000006715), FKTN(ENSG00000106692), FAM171A1(ENSG00000148468), GCNT3(ENSG00000140297), B4GALT4(ENSG00000121578), CACNA1B(ENSG00000148408), DOK7(ENSG00000175920), FZD2(ENSG00000180340), HLA-DQB1(ENSG00000179344), CLMN(ENSG00000165959), COQ2(ENSG00000173085), C6orf89(ENSG00000198663), DGAT1(ENSG00000185000), LIME1(ENSG00000203896), MALRD1(ENSG00000204740), ERBIN(ENSG00000112851), CHPF2(ENSG00000033100), SLC25A13(ENSG00000004864), ATL2(ENSG00000119787), CLCA4(ENSG00000016602), COG8(EENNSSGG0000000000221732368107), ANKRD46(ENSG00000186106), ATRN(ENSG00000088812), ADGRL1(ENSG00000072071), CDH26(ENSG00000124215), UGT8(ENSG00000174607), KCNA5(ENSG00000130037), GLCE(ENSG00000138604), SLC16A9(ENSG00000165449), KCNK10(ENSG00000100433), NOD2(ENSG00000167207), SLC16A4(ENSG00000168679), ITSN1(ENSG00000205726), CERS4(ENSG00000090661), SMAGP(ENSG00000170545), MIEF2(ENSG00000177427), TFPI(ENSG00000003436), SPCS3(ENSG00000129128), RNF139(ENSG00000170881), SUSD4(ENSG00000143502), NUCB2(ENSG00000070081), VPS11(ENSG00000160695), DOK7(ENSG00000175920), FZD2(ENSG00000180340), MALRD1(ENSG00000204740), ATRN(ENSG00000088812), BBS5(ENSG00000163093), SLC16A9(ENSG00000165449), VDAC3(ENSG00000078668), LRP1(ENSG00000123384), VAMP1(ENSG00000139190), SNX19(ENSG00000120451), SLC35E1(ENSG00000127526), COX14(ENSG00000178449), CYP26B1(ENSG00000003137), RNF139(ENSG00000170881), MAGI2(ENSG00000187391), CAPN2(ENSG00000162909), SMIM1(ENSG00000235169), MIEF2(ENSG00000177427), SRD5A3(ENSG00000128039), ATP6V1B1(ENSG00000116039), SRPRB(ENSG00000144867), TRPV5(ENSG00000127412), EMC10(ENSG00000161671), CDIPT(ENSG00000103502), SLC2A5(ENSG00000142583), CPM(ENSG00000135678), BBS5(ENSG00000163093), PDZD3(ENSG00000172367), SEZ6L2(ENSG00000174938), PRIMA1(ENSG00000175785), RHOA(ENSG00000067560), SUSD4(ENSG00000143502), KIFC3(ENSG00000140859), ENTPD1(ENSG00000138185), FIBP(ENSG00000172500), NLGN1(ENSG00000169760), CCR4(ENSG00000183813), SDR16C5(ENSG00000170786), RDH13(ENSG00000160439), RAB28(ENSG00000157869), PRKN(ENSG00000185345), PLPPR2(ENSG00000105520), TMEM106C(ENSG00000134291), VAMP5(ENSG00000168899), TOMM40L(ENSG00000158882), TMX2(ENSG00000213593), TSNARE1(ENSG00000171045), GRAP(ENSG00000154016), FAM162A(ENSG00000114023), AFG1L(ENSG00000135537), COMTD1(ENSG00000165644), PYCARD(ENSG00000103490), MAPK8IP1(ENSG00000121653), CAV2(ENSG00000105971), PPP1R16B(ENSG00000101445), SDR16C5(ENSG00000170786), RAB28(ENSG00000157869), TMX2(ENSG00000213593), TSNARE1(ENSG00000171045), CAV2(ENSG00000105971), MAP1LC3B(ENSG00000140941), GRAP(ENSG00000154016), FAM162A(ENSG00000114023), AFG1L(ENSG00000135537),
COMTD1(ENSG00000165644), PYCARD(ENSG00000103490), MAPK8IP1(ENSG00000121653), STBD1(ENSG00000118804), PPP1R16B(ENSG00000101445), MAP1LC3B(ENSG00000140941), RMDN2(ENSG00000115841), PLOD1(ENSG00000083444), SLC22A1(ENSG00000175003), SDCBP2(ENSG00000125775), GBA2(ENSG00000070610), GPR34(ENSG00000171659), Clorf210(ENSG00000253313), EPB41L2(ENSG00000079819), LBR(ENSG00000143815), MMP2(ENSG00000087245), PCDHB5(ENSG00000113209), FGFR2(ENSG00000066468), GPR34(ENSG00000171659), SDCBP2(ENSG00000125775), EPB41L2(ENSG00000079819), RETSAT(ENSG00000042445), FGFR2(ENSG00000066468), PCDHB5(ENSG00000113209), ZC3H12A(ENSG00000163874), IKBIP(ENSG00000166130), NUP54(ENSG00000138750)

### List of All Proteins in the List Above Separately Categorized by Applicable Use

(Ensembl version 9238 ID for the symbols below can be found in the table above):
1. Cancer treated: melanoma
   a. Tissue protected: Colon AXL, VPS52, SCGN, SLC9A2, PCSK5, SELENBP1, SNX24, SMIM24, SMURF1, NAAA, SEC13, SLC41A2, SOCS7, QRFPR, RPH3AL, RAC1, NFAM1, LDLR, GPR65, RAC3, WSCD1, HTRA1, RAB27B, DLC1, PLCB3, MUC1, PSTPIP2, RAB3B, SDC1, TMEM45A, SPPL3, STYK1, MAPKAP1, IFI27, ITLN1, PTGDR, SGMS1, ITGB6, MUC15, PEX10, OSBPL7, CD46, PROM2, PLPP7, MGAT4A, LRRN2, OBSCN, PROM1, VSIG10, TMEM236, VPS35L, SYTL4, SYTL3, TMEM41A, TSPAN8, UNC13B, SYTL2, TPRA1, OCLN, RNF170, SPHK1, SLC28A3, PTAFR, SYP, PIGZ, PTPRN2, RSAD2, SLC39A14, SLC6A12, SLC6A15, RAB25, SLC20A2, TGFBR1, STK16, TRAF4, SIGLEC14, MTG2, SPTLC3, MARK2, RNF186, TSPAN15, TRAF3IP3, VTCN1, TAS2R38, TMEM39B, CA2, CDHR5, CLDN4, ARL4D, ACVR1B, DPEP1, GSDMB, GPBAR1, SYK, ADCY10, ACSL5, CD177, CLCA1, GLP2R, DSC2, ADGRF1, CLDN5, DSG2, AQP8, CEACAM5, CHST6, CA4, EPCAM, GNG11, CTDNEP1, MAGI1, ENTPD8, FPGS, MAN1C1, FADS2, FER1L6, BEST2, BRI3, CLDN8, GAL3ST2, HTR4, B3GNT8, ELOVL7, B3GALT5, CDHR2, CLDN7, ACE2, CNTNAP3, CDH17, ATP11C, CIB1, ASIC5, DCXR, AKAP10, ARL4C, ANO1, CHST14, ABHD12, C12orf66, ALG14, HTR2B, B3GAT1, HHLA2, MAOA, FGFR4, IBTK, SLC9A3R2, NCEH1, KCNN3, MC4R, MCUB, EPPK1, MSTO1, SLC2A6, KIAA2013, NIPAL1, IL17RA, ITGB4, GPA33, MUC13, OR10R2, PSKH1, NDST2,
   b. Tissue protected: Lung TGM2, AXL, SLC30A4, VPS52, SLC22A15, SELENBP1, SCEL, SMURF1, NFAM1, AGER, RECK, MUC1, SCTR, ITGB6, CD46, EHD2, PRRG2, RNF170, RSAD2, SLC6A15, RAB25, SLC39A10, TM4SF1, STEAP1, CLDN4, EHD1, GPBAR1, CCR7, CYB5A, ACSL5, CLDN5, AIFM2, LPAR6, FADS2, AIG1, CLDN8, AQP4, CYB561D1, CACNA2D2, ACE, AQP3, CHST14, CADM1, MAOA, IBTK, NCEH1, LAMP3, MC4R, EHD3, NSMF, SLC34A2, PSKH1, LRRK2,
   c. Tissue protected: Skin VPS52, CLEC10A, PERP, SCEL, SLC41A2, RAC1, RNF145, NFAM1, RAC3, MTMR2, REEP2, TMEM134, SLC10A6, SGMS1, OLFM2, PROM2, KRT1, PLA2G4E, OBSCN, ZP2, SYTL3, TACSTD2, STK16, TRAF3IP3, GJB4, KCNJ8, SYK, FA2H, DSP, CD177, ANO7, CLDN5, EPHA4, CAPNS2, DSC3, GSDMA, FGFBP1, FADS3, FADS2, B3GNT4, DSG1, ADGRF4, ASPRV1, CIB1, DSG3, DCXR, AQP3, CNPPD1, EPPK1, FGFR3, OR10R2, PSKH1, VPS52, MS4A8, MGAT4C, SEC13, TMEM45A, IFI27, ICAM3, PLPP7, PPL, RHCG, TPRA1, RSAD2, S100A8, RAB25, MARK2, CLDN4, DSC2, GPX8, HCN1, CYB561D1, CLEC2A, HTR2B, MAOA, ITGB4, ALOX12, ,
   d. Tissue protected: Liver VPS52, CLEC10A, PCSK5, PI4KB, ALPL, SELENBP1, ST6GAL1, NAAA, SLC25A40, SLC27A2, SLCO1B3, SLCO1B1, SLC25A43, SLC27A5, SERINC3, NFAM1, LDLR, GPR65, PDZK1, RDH16, SDC1, TMEM45A, SPPL3, MAPKAP1, CD46, LRRN2, SLC25A15, UGT2B4, YIPF1, TFR2, VPS35L, UGT1A6, TNS2, UNC13B, PTAFR, RSAD2, SLC39A14, SLC6A12, TSPAN2, SLC30A10, CA2, ACVR1B, GHR, KCNJ8, CYP2D6, CYB5A, ACSL5, CD177, FYN, GAS2, SLC2A2, CYP2A7, CYP1A2, CYP3A4, CYP4V2, NINJ1, CYP2C8, DHCR24, CTDNEP1, CYP2C19, FADS2, AQP9, AIG1, BRI3, CDHR2, ASGR1, ABCB11, ASIC5, ACAD11, DCXR, ANO1, ASGR2, CYP3A43, INTS2, MAOA, CYP2C9, FGFR4, IBTK, LHFPL2, MFAP3L, MSTO1, SLC2A6, ITGB4, ABCC2, DPP4, PSKH1, NPC1L1,
   e. Tissue protected: Kidney (glomeruli) SLC9A2, SLC45A1, PODXL, NFAM1, RECK, TPSG1, PECAM1, SCUBE1, PLA2R1, CD46, PROM2, PNPLA3, MAP6, EHD2, NPHS2, TNS2, PRRG2, PTPRO, ARL4D, EHD1, HYAL2, ADGRF1, CD34, CLDN5, EVI2A, ENG, CR1, FADS3, CRB3, SLC9A3R2, EHD3, DPP4, PSKH1,
   f. Tissue protected: Kidney (tubules) TTYH3, AXL, TRPM3, VPS52, TREH, FCAMR, SLC9A2, TM4SF18, SELPLG, PCSK5, ALPL, SELENBP1, SMIM24, SLC5A12, ST6GAL1, SLC12A1, SMURF1, NAAA, SLC25A40, SLC27A2, SLC41A2, RPH3AL, SLC6A19, SLC22A11, S1PR2, SERINC3, ARHGAP5, NFAM1, LDLR, RHBG, PLLP, WSCD1, VHL, HTRA1, RAB27B, PARVB, DLC1, MUC1, PDZK1, KCNH8, LIN7A, SDC1, MGAT4B, TMEM132E, SPPL3, SCTR, MAPKAP1, SGMS1, MUC15, OSBPL7, CD46, OLFM2, PROM2, NAT8, PNPLA3, MGAT4A, LRRN2, MPP5, SLC25A15, OBSCN, PROM1, UGT2B4, SLC22A6, TMEM72, UGT3A1, VSIG10, ATP6V0A4, SLC7A7, TMEM80, VPS35L, ZP2, UGT1A6, TMEM252, TMEM174, TSPAN8, UNC13B, RHCG, SYTL2, SLC28A1, SLC22A2, OCLN, RNF170, SPHK1, SLC28A3, SCAMP5, PTAFR, SLC22A8, PIGZ, RSAD2, SLC39A14, SLC6A12, RAB25, RAB11FIP5, SLC20A2, SLC6A18, SLC13A2, TGFBR1, TMEM213, SYT7, SPTLC3, SLC4A4, MARK2, TMEM178B, PKHD1, SLC35G2, TSPAN2, VTCN1, TSPAN16, TMEM39B, CA2, CDHR5, ARL4D, ENPP6, GGT1, DPEP1, ENPP3, CUBN, GHR, HYAL2, KCNJ8, GSDMC, SYK, CPNE6, CYB5A, ADCY10, ACSL5, CDH6, GAS2, SLC2A2, CALCR, DSG2, CASR, FOLH1, CNTFR, EPCAM, GNG11, CTDNEP1, LRFN4, MAGI1, FADS2, AQP6, CDC42EP5, CLDN8, CDHR2, CLDN7, SH3BP5, SLC7A9, CLDN10, FXYD2, ACE, CMKLR1, CLDN2, DCXR, AKAP10, ARL4C, ANXA13, AQP3, C12orf66, CYP3A43, CDH16, CADM1, B3GAT1, INTS2, MAOA, FGFR4, MOXD1, LHFPL2, MC4R, KCND1, MCUB, MSTO1, SLC2A6, KIAA2013, NIPAL1, FMO1, LRP2, ALOX12, OR10R2, GRIN2D, NECAP2, NSMF, ABCC2, MDGA2, DPP4, PSKH1, LRRK2, NDST2,
   g. Tissue protected: Heart (cardiomyocytes) TGM2, TTYH3, SLC30A4, SLC27A6, UCP3, CD36, MGAT4C, RASL10B, SLC22A15, PCSK5, PI4KB, SNX24, SLC4A10, SLC41A2, RNF145, NFAM1, HTRA1, PARVB, POPDC2, PLN, SGCG, SRL, MAPKAP1, SGMS1, ITGB6, OLFM2, PLPP7, DMPK, SLC25A15, OBSCN, YRDC, TMEM143, ZP2, PRRG2, TPRA1, RAB11FIP4, S LC39A14, SLC6A12, SLC20A2, SLCO2B1, TRAF4, MARK2, TMEM178B, TEX261, SCN7A, TSPAN15, TMEM39B, DSP, CDH6, CDH15, DSC2, GNB5, RASGRP2, CAVIN4, DSG2, CHRNA1, CNTFR, CD248, GPAT2, CHST6, CYP4V2, CTDNEP1, LEPROTL1, FADS2, CDC42SE1, CDHR2, ADAM28, CNTNAP3, CSF3R, ATP11C, CDH13, CIB1, ADGRG6, ADRB1, ACAD11, ARL4C, AP4E1, CNPPD1, MAOA, NCEH1, MYZAP, MFAP3L, GPR182, MC4R, KCND1, MSTO1, GPR15, SLC16A10, ITGB4, OR10R2, PRKCA, DES, PSKH1,
   h. Tissue protected: Thyroid SLC30A4, VPS52, SLC9A2, TMEM100, SLC22A15, SELENBP1, SLC5A5, ST6GAL1, QRFPR, MPP7, RNF145, NFAM1, TPO, MAPKAP1, IFI27, MUC15, OSBPL7, CD46, OLFM2, PSD3, PROM2, MGAT4A, OBSCN, PROM1, VPS35L, SYTL4, SYTL3, TMEM41A, UNC13B, PRRG2, OCLN, RNF170, PTAFR, RSAD2, RAB25, SLC20A2, SLC39A10, TRAF4, MARK2, TSPAN15, TMEM39B, CLDN4, IYD, DUOX1, GHR, KCNJ8, SYK, ADCY10, CDH6, FYN, CLDN5, EPHA4, DSG2, HCN1, IPCEF1, EPCAM, LPAR6, CTDNEP1, MAGI1, FADS2, CLDN8, CLDN7, AAK1, DCXR, AKAP10, AP4E1, CDH16, CADM1, MAOA, IBTK, LHFPL2, LRP1B, MC4R, MSTO1, IL17RA, OR10R2, GRIN2D, PSKH1,
   i. Tissue protected: Pancreatic endocrine cells (Islets) SCGN, PI4KB, STX1A, NAAA, RTN1, SCG3, VHL, PTGDR2, RAB3B, PTPRN, CD46, MGAT4A, OBSCN, NECAB2, SLC17A6, SV2A, YRDC, TSPAN7, SLC30A8, PRRG2, SYP, PTPRN2, RSAD2, RAB25, SIGLEC14, TEX261, SLC35G2, DGCR2, CDH6, GNAO1, CLDN5, FAM169A, DSG2, CASR, CNTFR, CTDNEP1, CLDN7, ATP9A, CLU, FXYD2, ATP1A2, ASIC5, CADM1, GAD2, ALOX5, MAOA, MC4R, KCND1, MSTO1, OR10R2, MDGA2, DPP4, PSKH1,
   j. Tissue protected: Pancreatic exocrine cells VPS52, RASL10B, SELPLG, PCSK5, PI4KB, SMURF1, NAAA, SLC4A10, SEC13, RPH3AL, TMEM97, NFAM1, VHL, RAB27B, MUC1, SCTR, IFI27, SGMS1, MUC15, OLFM2, PROM2, MGAT4A, LRRN2, OBSCN, PROM1, VSIG10, YRDC, TMEM80, SYTL4, TSPAN8, SYTL2, TPRA1, OCLN, RNF170, SPHK1, SYCN, SLC28A3, RSAD2, SLC39A14, RAB25, SLC20A2, TM4SF1, SLCO2B1, STK16, MTG2, SPTLC3, SLC4A4, MARK2, PKHD1, SCN7A, TSPAN15, TAS2R38, CA2, CLDN4, ARL4D, ACVR1B, DPEP1, CUZD1, KCNJ8, GP2, SYK, KIRREL2, FA2H, ADCY10, CDH6, DSC2, CLDN5, EVI2A, DSG2, AQP8, CNTFR, GPAT2, CHST6, CYP4V2, LPAR6, CTDNEP1, MAGI1, LEPROTL1, FADS2, CLDN8, ELOVL7, CLDN7, CRB3, ACE2, CFTR, CLDN10, FXYD2, AKAP10, ARL4C, AP4E1, PIK3AP1, CADM1, MAOA, AQP12A, AQP12B, FGFR4, IBTK, MOXD1, MC4R, MCUB, SLC2A6, SLC16A10, IL17RA, OR10R2, PSKH1,
2. Cancer treated: lung cancer
   a. Tissue protected: Colon AXL, UGT2A3, CORO7, GABARAPL2, SCGN, PCSK5, SMIM24, SPCS1, TNFSF12, NAAA, PLEKHA3, TVP23B, WHAMM, DLC1, PLCB3, RAB3B, TMEM45A, SLCO4A1, STYK1, SLC9A3, ITLN1, PTGDR, PEX10, PCDH11X, PALM, PLPP7, VEPH1, CNEP1R1, GOLGA2, OBSCN, AATK, SLC26A3, UGT2B17, VSIG10, SERAC1, TVP23C, UGT2B28, TMEM253, UGT2B15, ARHGAP17, C2orf88, GOPC, SPHK1, PTPRN2, SLC39A14, SLC6A12, SNX6, PHACTR2, NAPG, NAPB, TRAF3IP3, TMEM171, TMEM39B, TMEM30A, CA2, DPEP1, CLSTN1, CLCA1, GLP2R, CEACAM1, FPR2, FKBP2, CHDH, FAM126B, HTR1A, GLT8D2, GATM, FLNC, AQP8, DIAPH1, CA12, CA4, DPP10, DGKQ, GBP2, LRRC37B, MAGI1, ENTPD8, MAN1C1, AP3M1, ANKRD27, ANO8, BEST2, BRI3, GAL3ST2, ELOVL7, CDHR2, ACE2, CNTNAP3, ARRB1, C5AR2, ASIC5, AKAP10, ABHD12, EBP, HSD3B7, ALG14, B3GAT1, IBTK, SMPD4, SLC16A6, GFRA2, MCUB, GNG12, GLRA2, SLC2A6, KIAA2013, NIPA2, NIPAL1, ITGB4, DPY19L1, RPS6KC1, OR10R2, PSKH1,
   b. Tissue protected: Lung AXL, SLC30A4, TRIM13, PLSCR4, SLC25A17, AGER, TVP23B, WHAMM, RECK, PTPRG, SGIP1, PALM, EHD2, SERAC1, TVP23C, PSD4, PHLPP2, EHD1, EHD4, CCR7, SLC44A2, AOC3, GLT8D2, GBP2, AIFM2, AP3M1, ANKRD27, ELMO2, CACNA2D2, ACE, ARRB1, IBTK, EHD3, NIPA2, LDLRAD2, PI4KA, PSKH1, LRRK2,
   c. Tissue protected: Skin CACNB2, CLEC10A, TGFBR3, REEP2, PALM, VEPH1, GOLGA2, OBSCN, AATK, TRPM2, RMC1, SERAC1, ZP2, C2orf88, GOPC, RAB29, TRAF3IP3, TMEM171, GJB4, DSP, ANO7, FAM126B, GATM, CD99, CAPNS2, CA12, GBP2, GSDMA, AP3M1, FADS3, B3GNT4, ARRB1, ASPRV1, DSG3, CNPPD1, RPS6KC1, OR10R2, PSKH1, MS4A8, CACNB2, MGAT4C, TNFSF12, TYR, TMEM45A, PLPP7, DCT, ZC4H2, RYK, PMEL, GPX8, CYB561A3, FAM210A, HCN1, MALL, BST1, HSD3B7, ITGB4, ALOX12, ,
   d. Tissue protected: Liver CACNB2, CLEC10A, TRIM13, SYT13, PCSK5, PI4KB, SVEP1, SPCS1, NAAA, SLC27A2, SLCO1B3, SLCO1B1, SLC27A5, TMEM45A, KCNQ4, VEPH1, SORD, MME, SLC25A15, AATK, SPRED1, UGT2B4, YIPF1, TFR2, SERAC1, TNS2, TBCD, SLC39A14, SLC6A12, TMEM171, CA2, FCER1A, CYP2D6, FYN, GAS2, SLC2A2, HTR1A, CYP2A7, GLT8D2, GATM, CYP1A2, GCHFR, AADAC, CYP3A4, DIAPH1, FAM210A, FMO3, GBP2, ABCB4, NINJ1, CYP2C8, FES, KMO, CYP2C19, BCL2L10, AQP9, BRI3, UGCG, CDHR2, ENPEP, ASGR1, ABCB11, CTSL, ASIC5, HSD11B1, EBP, ASGR2, CYP3A43, HSD3B7, INTS2, CYP2C9, IBTK, SLC16A6, LHFPL2, MFAP3L, SLC2A6, IGFLR1, ITGB4, DPY19L1, ABCC2, PSKH1, SLC10A1, NPC1L1,
   e. Tissue protected: Kidney (glomeruli) ZAP70, CORO7, KDR, MAPT, SYT13, TGFBR3, TNFSF12, SLC45A1, PODXL, KIRREL1, WHAMM, KSR2, RECK, PTPRG, MRGPRF, TPSG1, PECAM1, SCUBE1, PLA2R1, PTH2R, PNPLA3, PRKAR2B, EHD2, MME, NPHS2, RMC1, TNS2, A RHGAP17, SNCA, PSD4, PHACTR2, NAPG, RYK, SLC35B1, TMEM171, CLSTN1, EHD1, HYAL2, EHD4, CD34, GLT8D2, EVI2A, ENG, CD99, FAM210A, CR1, GBP2, FADS3, ANKRD27, ANO8, AQP1, ENPEP, ARRB1, LIMS2, ITGA8, EHD3, NIPA2, LDLRAD2, PI4KA, PSKH1,
   f. Tissue protected: Kidney (tubules) VNN1, AXL, TRPM3, TREH, CACNB2, ABCD4, FCAMR, GABARAPL2, TM4SF18, UBE2J1, SELPLG, MAPT, PCSK5, SVEP1, SMIM24, SLC5A12, SLC5A2, SPCS1, TNFSF12, SLC12A1, NAAA, SLC27A2, SLC6A19, S1PR2, SLC34A3, NEGR1, PTH1R, WHAMM, PTPRD, DLC1, MPP6, KCNH8, LIN7A, MRGPRF, SLC34A1, PRICKLE1, SLCO4A1, KCNQ4, NAT8, PNPLA3, PALM, VEPH1, KIRREL3, CNEP1R1, SORD, GOLGA2, MPP5, MME, SLC25A15, OBSCN, AATK, UGT2B4, SLC22A6, TMEM72, UGT3A1, XPNPEP2, VSIG10, ATP6V0A4, SLC7A7, SERAC1, ZP2, VAC14, QTRT1, TMEM252, TSPAN13, TMEM174, TTC7B, ARHGAP17, SLC28A1, SLC22A2, GOPC, SPHK1, SCAMP5, SLC22A8, SLC13A3, SLC39A14, SLC6A12, SNX6, PHACTR2, RAB11FIP5, SLC6A18, SLC13A2, TMEM213, SYT7, RAB29, PEAR1, SLC4A4, NAPG, RYK, PKHD1, PRRT3, SLC35G2, OPRD1, NAPB, TMEM171, TSPAN16, TMEM39B, TMEM30A, CA2, ENPP6, GGT1, DPEP1, ENPP3, CUBN, CLSTN1, FCER1A, HYAL2, SLC44A2, ADCY7, FAM151A, GAS2, FKBP2, SLC2A2, CHDH, FAM126B, HTR1A, CREB3L2, GLT8D2, TM7SF2, GATM, CASR, FOLH1, GCHFR, FAM210A, CA12, DGKQ, GBP2, FES, KMO, LRFN4, L1CAM, MAGI1, AP3M1, ANKRD27, BSND, BCL2L10, ANO8, AQP6, AQP1, CDC42EP5, MPP1, UGCG, CDHR2, SH3BP5, ENPEP, SLC7A9, FXYD2, ACE, CMKLR1, COX7B, AKAP10, ANXA13, AP1S3, EPB41L3, CYP3A43, HSD3B7, CDH16, B3GAT1, INTS2, ABCC5, SMPD4, SLC43A2, SLC16A6, LHFPL2, NDUFS1, GFRA2, MCUB, GNG12, SLC2A6, KIAA2013, NIPA2, NIPAL1, FMO1, GAL3ST1, DPY19L1, LRP2, ALOX12, CHL1, PI4KA, OR10R2, GRIN2D, ABCC2, PSKH1, LRRK2,
   g. Tissue protected: Heart (cardiomyocytes) TCTN2, SLC30A4, CAP2, CD36, GABARAPL2, MGAT4C, RASL10B, PCSK5, SLC25A17, PI4KB, TNFSF12, TLN2, SGCB, NEGR1, BVES, P2RX6, POPDC2, P2RY6, PLN, SGCG, SLCO4A1, SRL, KCNQ4, LANCL2, PLPP7, VEPH1, CNEP1R1, DMPK, GOLGA2, SLC25A15, OBSCN, AATK, SIPA1, SERAC1, YRDC, ZP2, VAC14, TBC1D3, ARHGAP17, SLC25A25, C2orf88, RAB11FIP4, SLC39A14, SLC6A12, SLCO2B1, RAB29, TEX261, TMEM171, TMEM39B, TMEM30A, CNTN3, CKMT2, DSP, ADCY7, CACNA2D1, CDH15, FNDC5, GNB5, FAM126B, HTR1A, CAVIN4, SLC2A4, GLT8D2, HHATL, FLNC, CHRNA1, CD248, GPAT2, DGKQ, GBP2, CAMK2B, LRRC37B, FES, LEPROTL1, AP3M1, ABHD16A, ANKRD27, DMD, CDHR2, CNTNAP3, CSF3R, CPT1B, COX7B, CDH13, AKAP6, ADGRG6, ADRB1, AP1S3, CNPPD1, ABCC5, SMPD4, SLC16A6, MYZAP, MFAP3L, NCAM1, GPR182, NDUFS1, GPR15, MLIP, IGFLR1, ITGB4, LDLRAD2, DPY19L1, PI4KA, OR10R2, PRKCA, DES, PSKH1,
   h. Tissue protected: Thyroid SLC30A4, TMEM100, VPS33B, TGFBR3, TNFSF12, SCNN1D, NUS1, TVP23B, LANCL2, PSD3, VEPH1, CNEP1R1, SORD, GOLGA2, OBSCN, AATK, SERAC1, TVP23C, TSPAN13, TBC1D3, TBCD, ZC4H2, ARHGAP17, GOPC, RNFT2, NAPG, RYK, TMEM39B, TMEM30A, IYD, DUOX1, CLSTN1, FYN, FKBP2, FAM126B, GLT8D2, CD99, DIAPH1, HCN1, MALL, FES, MAGI1, AP3M1, ANKRD27, AAK1, ARRB1, ARRDC1, AKAP10, AP1S3, CDH16, IBTK, MTNR1B, LHFPL2, NCAM1, GFRA2, GNG12, NIPA2, IGFLR1, FAM234B, OR10R2, GRIN2D, PSKH1,
   i. Tissue protected: Pancreatic endocrine cells (Islets) CACNB2, SCGN, PLSCR4, SLC25A17, TGFBR3, PI4KB, SVEP1, STX1A, NAAA, SCG3, TVP23B, MPP6, PTGDR2, RAB3B, KCNQ4, PTPRN, PTH2R, VEPH1, GOLGA2, OBSCN, AATK, NECAB2, SLC17A6, SHISAL2B, SERAC1, YRDC, TVP23C, VAC14, TSPAN7, TSPAN13, TBC1D3, SLC30A8, SLC25A25, PTPRN2, PHACTR2, NAPG, TEX261, SLC35G2, NAPB, DGCR2, FAM169A, CASR, CD99, DIAPH1, CD82, GBP2, UGCG, ELMO2, CLU, FXYD2, CADPS, ARRDC1, ASIC5, EPB41L3, GAD2, ALOX5, IGFLR1, LDLRAD2, PI4KA, OR10R2, PSKH1,
   j. Tissue protected: Pancreatic exocrine cells GABARAPL2, RASL10B, UBE2J1, SELPLG, PCSK5, PLSCR4, PI4KB, SVEP1, SPCS1, STIMATE, NAAA, TMEM97, PLEKHA3, TVP23B, MED28, MPP6, PTH2R, NEURL1, VEPH1, GOLGA2, OBSCN, AATK, SPRED1, VSIG10, SERAC1, YRDC, TVP23C, VAC14, TSPAN13, TBC1D3, ZC4H2, GOPC, SPHK1, SYCN, SLC39A14, PHACTR2, SLCO2B1, RAB29, SLC4A4, NAPG, RYK, PKHD1, CA2, DPEP1, CUZD1, KIRREL2, ADCY7, FKBP2, GLT8D2, EVI2A, GATM, AQP8, DIAPH1, FAM210A, GPAT2, CA12, DGKQ, GBP2, FES, MAGI1, LEPROTL1, AP3M1, AQP1, CDC42EP1, ELOVL7, ACE2, CFTR, FXYD2, ARRB1, ARRDC1, AKAP10, PIK3AP1, AQP12A, AQP12B, IBTK, SMPD4, MCUB, SLC2A6, NIPA2, FAM234B, OR10R2, PSKH1,
3. Cancer treated: renal cancer
   a. Tissue protected: Colon TJAP1, UGT2A3, TMEM138, TMEM237, SLC7A6, ATP8B2, SLC46A1, ST3GAL6, PCSK5, LPCAT2, SMIM24, SLC6A6, SEC13, SLC41A2, SOCS7, PEX1, PEMT, QRFPR, RPH3AL, NPDC1, NTRK1, GOLGA8B, GRIK2, RAC1, PCDHGA10, HRAS, PILRA, RAC3, PKHD1L1, USP8, YIF1B, WHAMM, PLCB3, RHBDL2, SLC25A20, PSTPIP2, RAB3B, PLA2G2A, KRAS, TMEM45A, STXBP6, NRAS, STYK1, SLC9A3, IFI27, ITLN1, TENM3, STXBP3, TAOK2, MITD1, MUC15, PRSS8, PEX10, GNPNAT1, OSBPL7, CD46, LRRC55, PROM2, NUTF2, PALM, PLPP7, KCNQ1, NECTIN1, OBSCN, NALCN, SLC51B, SLC26A3, SLC43A3, SELENOT, SLC9A1, ST7, SNTA1, UGT2B17, VSIG10, TMEM236, TUB, TEX264, VPS35L, UGT2B28, ZNRF1, TMEM69, TMEM253, TMEM41A, TSPAN8, ZFYVE9, UGT2B15, SLC35A3, GOPC, PCDH1, RNF125, QPCTL, SLC28A3, SLC45A4, RAB27A, SYP, SLC12A6, PARD3B, PIGZ, PTPRN2, SNX6, SMIM6, PHACTR2, MIA3, STK17B, SIGLEC14, SSTR1, TMEM204, SNX18, PIK3R5, RHOU, SLC4A5, MSMO1, NAPB, RNF186, TRAF3IP3, TMIGD2, TYRO3, TAS2R38, CA2, PRKCZ, ACVR1B, DPEP1, EMC9, FAM83B, GSDMB, GPBAR1, FKRP, FMO5, EMC6, SYK, KCTD3, GOLPH3, ATP2C2, ABCG2, CD177, CLCA1, GLP2R, CEACAM1, EPHB3, ADGRF1, FAM126B, DGKE, CYP4F11, GBF1, DMTN, GOLGA8A, AQP8, CEACAM5, WDPCP, CNR1, ARFGEF3, CARMIL2, CA4, CHMP5, KPNA2, GNG11, GPR82, IGSF11, IL17RC, LRRC37B, EVA1B, MAGI1, FPGS, LPP, MAN1C1, EPHA10, FER1L6, BEST2, BRI3, GAL3ST2, FERMT1, ABCA10, CNTNAP3, CDH17, DTX3L, CYB561, AVEN, CXADR, GRAMD1A, CIB1, DCXR, ARL4C, ABHD12, EBP, B3GNT7, ALG14, CHMP2B, ALDH3A2, IBTK, SMPD4, SLC9A3R2, NISCH, SLC16A6, KCNN3, GIPC1, FUT3, GFRA2, GLUL, IL17RE, MCUB, MARCKSL1, EFHD2, FUT6, GPER1, HCN3, MBTPS1, JAML, NIPAL1, IL17RA, ITGB4, GPA33, GPC1, RPS6KC1, LZTR1, MUC13, NEU4, OR10R2, MUC4, NAPEPLD, IL4R,
   b. Tissue protected: Lung SLC30A4, ST3GAL6, TRIM13, LPCAT2, WHAMM, PTPRG, STX5, CD46, PALM, KCNQ1, EHD2, NECTIN1, RNF125, PSD4, TM4SF1, STEAP1, S100A9, PHLPP2, RHOU, EHD1, GPBAR1, ARHGEF1, CCR7, SLC44A2, GPC5, DCBLD1, AIFM2, CLIC2, CYB561D1, DTX3L, CYB561, CACNA2D2, ACE, IBTK, LAMP3, EFHD2, EHD3, GPER1, NSMF,
   c. Tissue protected: Skin TMEM243, TJAP1, TMEM138, TMEM237, CLEC10A, PERP, RXFP1, SLC5A6, SLC6A6, SLC41A2, GRIK2, RAC1, RAC3, TMEM40, MTMR2, REEP2, TMEM134, SLC10A6, OLFM2, PROM2, PALM, LYPD3, KRT1, PLA2G4E, SLC8A1, OBSCN, SLC43A3, TRPM2, SLC9A1, SNTA1, SRC, TUB, ZP2, ZNRF1, TMEM69, THBD, TACSTD2, SLC35A3, GOPC, RNF125, TMTC4, QPCTL, PARD3B, SNX18, LY6D, S100A9, MSMO1, TRAF3IP3, EMC9, FAM83B, GJB4, SYK, FA2H, DSP, CD177, ANO7, FRS3, EPHB3, FAM126B, EPHA4, DMTN, CD99, CAPNS2, ARFGEF3, KPNA2, DSC3, GSDMA, FGFBP1, EVA1B, LPP, FADS3, EPHA10, B3GNT4, DSG1, FERMT1, ABCA10, CDH5, AVEN, CXADR, ASPRV1, CIB1, ORAI1, DSG3, DCXR, CNPPD1, ALDH3A2, NISCH, MARCKSL1, FGFR3, GPER1, TACR1, GPC1, RPS6KC1, OR10R2, TMEM243, MS4A8, MGAT4C, TYR, SEC13, NTRK1, GOLGA8B, PKHD1L1, TMEM45A, IFI27, STXBP3, ICAM3, LRRC55, PLPP7, KCNQ1, NECTIN1, PPL, ST7, DCT, SMIM6, PMEL, RHOU, TYRO3, KCTD3, ATP2C2, GPC5, GBF1, CYB561A3, FAT2, GOLGA8A, FAM210A, CARMIL2, MALL, FAM241B, IL17RC, CYB561D1, CLEC2A, GIPC1, ITGB4, ALOX12, ,
   d. Tissue protected: Liver TMEM138, TECR, TMCC1, CLEC10A, ST3GAL6, TRIM13, PCSK5, SLC25A40, SLCO1B3, SLCO1B1, SLC27A5, SERINC3, SLC25A20, RDH16, TMEM45A, STX5, TAOK2, GNPNAT1, CD46, LRRC55, KCNQ1, SLC8A1, SORD, RFTN1, TFR2, TUB, VPS35L, ZNRF1, TNS2, TMEM204, MSMO1, TM4SF4, SLC30A10, CA2, ACVR1B, FMO5, CYP2W1, CYP2D6, CD177, CACNA1A, FYN, GAS2, CYP4F3, CYP2A7, EPHX1, CYP4F11, CYP1A2, CYP3A4, FAM210A, CYP2A6, CYP4V2, KPNA2, NINJ1, CYP2C8, DHCR24, CYP2C19, AQP9, ATP5F1C, BRI3, CYP4F2, ASGR1, CDH5, ABCB11, CYP2A13, CYP2E1, CXADR, DCXR, EPB41L5, HSD11B1, EBP, ASGR2, CYP3A43, CHMP2B, ALDH3A2, KCNJ11, CYP2C9, IBTK, SLC16A6, LHFPL2, MFAP3L, GIPC1, GLUL, MTCH2, IGFLR1, ITGB4, ABCC2, NPC1L1,
   e. Tissue protected: Kidney (glomeruli) TMEM138, TECR, TMCC1, ZAP70, ST3GAL6, KDR, SLC45A1, PODXL, PDGFRB, TRPV6, YIF1B, WHAMM, KSR2, PTPRG, MRGPRF, TPSG1, STX5, PECAM1, SCUBE1, PLA2R1, CD46, PTH2R, MCAM, NEU3, KCNAB1, PROM2, NUTF2, MAP6, EHD2, NECTIN1, LDLRAD3, NPHS2, ST7, SMIM18, TNS2, SNCA, PSD4, SORT1, PARD3B, PHACTR2, SNX18, PTPRO, TYRO3, ZBED3, PRKCZ, EHD1, ATP2C2, DCBLD1, ADGRF1, CD34, EVI2A, DMTN, ENG, CD99, FAM210A, CR1, ARFGEF3, FAM241B, GPR82, GPRC5B, EVA1B, LPP, FADS3, CRB3, DTX3L, CXADR, EPB41L5, SLC9A3R2, LIMS2, GPR153, IL17RE, ITGA8, MARCKSL1, EFHD2, EHD3, GPER1, LZTR1, NFXL1, KIAA0040, NEU4,
   f. Tissue protected: Kidney (tubules) TMEM243, TJAP1, TRPM3, TMEM237, TMCC1, TREH, ST3GAL6, STRA6, UBE2J1, SELPLG, PCSK5, SMIM24, SLC6A6, SLC12A1, SLC25A40, SLC41A2, PEX1, PEMT, RPH3AL, SLC34A3, GRIK2, SERINC3, HRAS, PTH1R, PKHD1L1, RHBG, RGS16, USP8, VPS26B, YIF1B, WHAMM, PTPRD, PARVB, RHBDL2, SLC25A20, KRAS, LIN7A, MRGPRF, STXBP6, MGAT4B, SLC34A1, RASA4, STX5, PRICKLE1, NRAS, TENM3, MITD1, MUC15, GNPNAT1, OSBPL7, CD46, NEU3, OLFM2, LRRC55, PROM2, NUTF2, PALM, KCNQ1, SLC8A1, SORD, MPP5, NECTIN1, OBSCN, LDLRAD3, RFTN1, SLC9A1, UGT3A1, ST7, SNTA1, VSIG10, ATP6V0A4, SLC7A7, SRC, TMEM80, VPS35L, ZP2, ZNRF1, QTRT1, TMEM252, TMEM174, TTC7B, TSPAN8, ZFYVE9, SLC22A2, SLC52A3, GOPC, PCDH1, SLC28A3, SLC45A4, SCAMP5, SLC22A8, PARD3B, PIGZ, SNX6, SMIM6, PHACTR2, SLC6A18, SLC13A2, TMEM213, MIA3, STK17B, TMEM204, TMEM184A, SLC4A4, SNORC, SLC23A1, PIK3R5, PKHD1, RHOU, SLC35G2, SLC4A5, OPRD1, NAPB, TMEM242, TMIGD2, TYRO3, TSPAN16, CA2, ENPP6, DPEP1, EMC9, FKRP, EMC6, GSDMC, SYK, CPNE6, ATP2C2, SLC44A2, GJA5, GPC5, ADCY7, GAS2, EPHB3, FAM126B, TM7SF2, CASR, CYP4F11, FOLH1, GBF1, DMTN, FAM210A, CARMIL2, CHMP5, GNG11, GPR82, GPRC5B, IGSF11, LRFN4, SLC7A8, MAGI1, BSND, AQP6, ATP5F1C, CDC42EP5, MPP1, CYP4F2, FERMT1, CDH5, CLDN10, SLC25A31, ACE, COX7B, GRAMD1A, DCXR, ARL4C, ATP12A, CYP3A43, CHMP2B, ALDH3A2, ABCC5, SMPD4, NISCH, SLC16A6, LHFPL2, GIPC1, NDUFS1, GFRA2, IL17RE, MCUB, MTCH2, GPER1, MBTPS1, NIPAL1, FMO1, GPC1, LZTR1, ALOX12, NXPE2, OR10R2, NSMF, ABCC2, NAPEPLD,
   g. Tissue protected: Heart (cardiomyocytes) TJAP1, TCTN2, SLC30A4, TMEM138, TECR, TMCC1, SLC7A6, CD36, MGAT4C, RASL10B, ST3GAL6, SLC14A1, TMEM106B, PCSK5, SUSD3, SLC6A6, TLN2, PITPNM1, SLC41A2, PEMT, GRIK2, PKHD1L1, YIF1B, PARVB, POPDC2, P2RY6, SLC25A20, PLN, SGCG, SRL, MITD1, OLFM2, LRRC55, PLPP7, KCNQ1, SLC8A1, DMPK, NECTIN1, OBSCN, SLC43A3, SIPA1, RYR2, ST7, SNTA1, YRDC, TMEM143, TMEM119, ZP2, ZNRF1, TBC1D3, SLC25A25, SLC45A4, RAB11FIP4, SORT1, SLCO2B1, STK17B, TMEM204, PPM1L, TEX261, RHOU, MSMO1, CNTN3, EMC9, EMC6, CYP2W1, DSP, ADCY7, CDH15, DCBLD1, FNDC5, FAM126B, RASGRP2, CAVIN4, SLC2A4, DGKE, HHATL, FAM168B, GBF1, CHRNA1, CD248, WDPCP, GPAT2, ARFGEF3, CYP4V2, KPNA2, FAM241B, GPR82, CAMK2B, IGSF11, IL17RC, LRRC37B, EVA1B, ABHD16A, EPHA10, ATP5F1C, DMD, CDC42SE1, ATP1A3, CNTNAP3, DTX3L, CSF3R, ATP2A2, AVEN, CAV3, CPT1B, COX7B, CDH13, CIB1, ORAI1, AKAP6, ADGRG6, ARL4C, CNPPD1, ALDH3A2, ABCC5, SMPD4, NISCH, SLC16A6, MYZAP, MFAP3L, GIPC1, GPR182, NDUFS1, IL17RE, KIAA1109, MTCH2, GPER1, GPR15, HCN3, MBTPS1, MLIP, IGFLR1, ITGB4, NFXL1, OR10R2, NAPEPLD, DES,
   h. Tissue protected: Thyroid TJAP1, SLC30A4, TMEM237, TMCC1, TMEM100, TMEM106B, VPS33B, LPCAT2, SLC6A6, PEMT, QRFPR, NPDC1, NTRK1, GRIK2, MPP7, TPO, TRPV6, VPS26B, RHBDD2, STXBP6, IFI27, MUC15, OSBPL7, CD46, NEU3, OLFM2, PSD3, LRRC55, PROM2, NUTF2, KCNQ1, SLC8A1, SORD, NECTIN1, OBSCN, SLC43A3, SLC9A1, ST7, TUB, VPS35L, TBC1D3, TMEM41A, ZFYVE9, GOPC, RNF125, TMTC4, SLC45A4, SLC12A6, SORT1, SMIM6, MIA3, PPM1L, SNX18, PIK3R5, RHOU, TYRO3, IYD, DUOX1, EMC9, SYK, KCTD3, CYP2W1, FYN, EPHB3, FAM126B, EPHA4, DGKE, GBF1, CD99, CARMIL2, IPCEF1, KPNA2, MALL, GDAP1, IL17RC, EVA1B, MAGI1, CLIC2, FZD5, FERMT1, ABCA10, DTX3L, AVEN, CD300A, CXADR, DCXR, ALDH3A2, IBTK, NISCH, MTNR1B, LHFPL2, GIPC1, GFRA2, GLUL, IL17RE, KIAA1109, MARCKSL1, HCN3, MBTPS1, IGFLR1, IL17RA, OR10R2, NAPEPLD,
   i. Tissue protected: Pancreatic endocrine cells (Islets) STXBP1, STX1A, RTN1, NPDC1, NTRK1, SCG3, PTGDR2, RHBDL2, RAB3B, TAOK2, PTPRN, CD46, PTH2R, LRRC55, SLC8A1, OBSCN, NECAB2, SLC17A6, SV2A, YRDC, ZNRF1, TMEM69, TSPAN7, TBC1D3, ZFYVE9, SLC30A8, SLC25A25, SYP, SLC12A6, PTPRN2, PHACTR2, SIGLEC14, PPM1L, TEX261, SLC35G2, MSMO1, NAPB, DGCR2, CYP2W1, GNAO1, FAM169A, GCSAM, CASR, CD99, CD82, ARFGEF3, CHMP5, IL17RC, SLC7A8, DGKZ, FERMT1, CDH5, CYB561, ATP1A2, CADPS, AVEN, GAD2, KCNJ11, GIPC1, GPER1, IGFLR1, OR10R2, NAPEPLD,
   j. Tissue protected: Pancreatic exocrine cells TJAP1, TMEM237, RASL10B, UBE2J1, SELPLG, PCSK5, SLC6A6, STIMATE, SEC13, PEMT, RPH3AL, TMEM97, NTRK1, YIF1B, RHBDL2, SLC25A20, STX5, IFI27, TENM3, STXBP3, MITD1, MUC15, GNPNAT1, PTH2R, NEURL1, OLFM2, LRRC55, PROM2, NUTF2, KCNQ1, NECTIN1, OBSCN, SELENOT, ST7, SNTA1, VSIG10, YRDC, TMEM80, ZNRF1, TBC1D3, TSPAN8, GOPC, TMTC4, SYCN, SLC28A3, SLC12A6, PARD3B, PHACTR2, TM4SF1, MIA3, SLCO2B1, SSTR1, TMEM204, SLC4A4, PKHD1, RHOU, SELENOK, TAS2R38, CA2, PRKCZ, ACVR1B, DPEP1, EMC9, GP2, SYK, KCTD3, FA2H, GOLPH3, ADCY7, EVI2A, EPHX1, AQP8, ATP11B, FAM210A, WDPCP, GPAT2, ARFGEF3, CHMP5, CYP4V2, KPNA2, GPRC5B, MAGI1, CDC42EP1, KDELR3, FERMT1, CRB3, ABCA10, CFTR, CLDN10, CYB561, AVEN, CD300A, CXADR, ARL4C, B3GNT7, AQP12A, AQP12B, IBTK, SMPD4, GIPC1, IL17RE, KIAA1109, MCUB, MARCKSL1, EFHD2, GPER1, MBTPS1, IL17RA, MYOC, OR10R2,
4. Cancer treated: urothelial cancer
   a. Tissue protected: Colon UGT2A3, TMEM60, SCGN, SLC47A1, PCSK5, SMIM24, SH3BP4, SLC19A3, PILRA, DLC1, RNF144B, RAB3B, SLCO4A1, STYK1, SLC9A3, IFI27, ITLN1, PTGDR, SYBU, STXBP3, MITD1, PEX10, OSBPL7, MYORG, MCOLN1, LRRN2, OBSCN, AATK, UGT2B17, VSIG10, TMEM236, UGT2B28, SLC16A14, TMEM253, UNC13B, UGT2B15, SLC35A3, RAB27A, RAB43, PTPRN2, SNX6, PIK3R5, NAPB, RNF186, TMEM219, TRAF3IP3, TMEM39B, CRK, CA2, PRKCZ, DPEP1, GSDMB, ADCY10, BMPR1A, CLCA1, GLP2R, FPR2, FKBP2, CHDH, ADGRF1, FAM126B, HTR1A, CA4, GNG11, KCNMA1, LRRC37B, EVA1B, MAGI1, BEST2, ABCG8, BCL2, GAL3ST2, HTR4, B3GNT8, ELOVL7, CDHR2, ANPEP, CNTNAP3, CDH17, GRAMD1A, ASIC5, ABHD12, C12orf66, ALG14, ALDH3A2, B3GAT1, IBTK, MCUB, NIPAL4, GPA33, RPS6KC1, LZTR1, MUC13, HPS6, NEU4, OR10R2,
   b. Tissue protected: Lung SLC30A4, ILK, TRIM13, SLC22A15, SLC25A17, IL10RB, RNF144B, PTPRG, SGIP1, MXRA7, EHD2, PRRG2, PSD4, RAB43, STEAP1, EHD1, CCR7, SLC44A2, AOC3, AIFM2, CLIC2, AQP4, CACNA2D2, ACE, CADM1, IBTK, LAMP3, EHD3, NSMF, LRRK2,
   c. Tissue protected: Skin CACNB2, CLEC10A, PERP, ENPP1, REEP2, F2R, MCOLN1, OBSCN, AATK, TRPM2, RMC1, SLC16A14, SLC35A3, TRAF3IP3, CRK, FA2H, FAM126B, ATRAID, EVA1B, B3GNT4, DSG1, ABCG8, ASPRV1, DSG3, ALDH3A2, RPS6KC1, OR10R2, MS4A8, CACNB2, MGAT4C, TYR, SH3BP4, IFI27, STXBP3, DCT, PMEL, ARHGAP21, TMEM219, CYB561A3, MALL, BCL2, BBS1, ,
   d. Tissue protected: Liver APLNR, CACNB2, CLEC10A, TRIM13, SYT13, PCSK5, RAB17, ENPP1, SLC27A2, SLCO1B3, SLCO1B1, SLC27A5, SLC19A3, PEX11G, RNF144B, KCNQ4, MYORG, MCOLN1, LRRN2, SLC25A15, AATK, UGT2B4, TFR2, TNS2, UNC13B, RAB43, TSPAN2, TM4SF4, CRK, CA2, CYP2W1, CYP2D6, BMPR1A, CACNA1A, FYN, GAS2, SLC2A2, HTR1A, AKR1A1, CYP2A7, CYP1A2, CYP3A4, CYP2A6, CYP2B6, FMO3, NINJ1, CYP2C8, FES, KMO, CYP2C19, BCL2L10, AQP9, ABCG8, AP1S2, CDHR2, ANPEP, ASGR1, CYP2A13, ASIC5, ACAD11, BBS1, ASPHD1, HSD11B1, ASGR2, CYP3A43, ALDH3A2, INTS2, CYP2C9, IBTK, MFAP3L, NIPAL4, IGFLR1, HPS6, ABCC2, DPP4, SLC10A1, NPC1L1,
   e. Tissue protected: Kidney (glomeruli) ZAP70, ILK, MAPT, SYT13, RAB17, SLC45A1, KIRREL1, KSR2, PTPRG, MRGPRF, TPSG1, PLA2R1, PTH2R, F2R, MAP6, EHD2, LDLRAD3, NPHS2, RMC1, S LC16A14, TNS2, PRRG2, SNCA, PSD4, PTPRO, SREBF1, VASN, PRKCZ, EHD1, AIF1L, ADGRF1, CD34, ENG, CR1, GPRC5B, EVA1B, AQP1, ABCG8, CRB3, ABCC1, LIMS2, ITGA8, EHD3, NIPAL4, LZTR1, HPS6, NEU4, DYSF, DPP4,
   f. Tissue protected: Kidney (tubules) VNN1, TRPM3, TREH, CACNB2, ILK, STRA6, UBE2J1, SLC47A1, SELPLG, MAPT, PCSK5, RAB17, ENPP1, SMIM24, SLC5A12, SLC5A2, SLC12A1, SLC27A2, S1PR2, SLC34A3, SLC19A3, MGAM, PEX11G, PTH1R, VPS26B, PARVB, DLC1, RNF144B, LIN7A, MRGPRF, TMEM132E, PRICKLE1, SLCO4A1, TMEM52B, KCNQ4, MITD1, OSBPL7, NAT8, MYORG, MCOLN1, KIRREL3, LRRN2, MPP5, SLC25A15, OBSCN, LDLRAD3, AATK, UGT2B4, ATP6V1D, SLC22A6, TMEM72, UGT3A1, VSIG10, ATP6V0A4, SLC7A7, UST, QTRT1, TMEM252, SLC16A14, TMEM174, UNC13B, SLC28A1, SLC22A2, SCAMP5, SLC22A8, SLC13A3, RUFY3, SNX6, SLC6A18, SLC13A2, TMEM213, SLC4A4, SREBF1, PIK3R5, PKHD1, ARHGAP21, SLC35G2, OPRD1, NAPB, TSPAN2, TMEM219, VASN, TMEM39B, CRK, CA2, GGT1, DPEP1, ENPP3, CUBN, SLC44A2, AIF1L, ADCY10, CDH6, FAM151A, AP4M1, GAS2, FKBP2, SLC2A2, CHDH, FAM126B, HTR1A, AKR1A1, TM7SF2, ATRAID, FOLH1, CLIC4, GNG11, KCNMA1, GPRC5B, FES, KMO, MAGI1, BCL2L10, AQP1, ABCG8, CDC42EP5, BCL2, MPP1, AP1S2, CDHR2, ANK2, ANPEP, SLC7A9, CLDN10, FXYD2, ACE, CLDN2, GRAMD1A, ANXA13, BBS1, ASPHD1, C12orf66, CYP3A43, CDH16, CADM1, ALDH3A2, B3GAT1, INTS2, NDUFS1, KCND1, MCUB, GAL3ST1, LRP2, LZTR1, HPS6, OR10R2, GRIN2D, NSMF, ABCC2, MDGA2, DYSF, DPP4, LRRK2,
   g. Tissue protected: Heart (cardiomyocytes) TCTN2, SLC30A4, UCP3, APLNR, CD36, ILK, MGAT4C, TMEM106B, SLC22A15, PCSK5, SLC25A17, RAB17, SLC19A3, BVES, P2RX6, PARVB, POPDC2, P2RY6, PLN, RNF144B, SGCG, SLCO4A1, SRL, KCNQ4, MITD1, MXRA7, LANCL2, MYORG, MCOLN1, SLC25A15, OBSCN, AATK, SIPA1, RYR2, YRDC, UST, TBC1D3, PRRG2, SLC25A25, RAB11FIP4, SLCO2B1, ST8SIA2, TEX261, SCN7A, ARHGAP21, TMEM219, VASN, TMEM39B, CKMT2, CYP2W1, BMPR1A, CDH6, CACNA2D1, CDH15, FAM126B, HTR1A, CAVIN4, HHATL, CHRNA1, GPAT2, CAMK2B, LRRC37B, EVA1B, FES, LEPROTL1, ABCG8, AP1S2, CDHR2, ANK2, CNTNAP3, CPT1B, CDH13, AKAP6, ADGRG6, ADRB1, ACAD11, BBS1, AP4E1, PAM, ALDH3A2, MYZAP, MFAP3L, NCAM1, GPR182, NDUFS1, KCND1, GPR15, NIPAL4, IGFLR1, OR10R2, DES, DYSF,
   h. Tissue protected: Thyroid SLC30A4, APLNR, ILK, TMEM100, TMEM106B, SLC22A15, RAB17, ENPP1, SCNN1D, SLC19A3, TPO, VPS26B, RHBDD2, IFI27, SYBU, MXRA7, OSBPL7, LANCL2, PSD3, MCOLN1, OBSCN, AATK, UST, SLC16A14, TBC1D3, UNC13B, PRRG2, RAB43, SLC26A4, SREBF1, PIK3R5, ARHGAP21, TMEM39B, CSK, IYD, CYP2W1, ADCY10, BMPR1A, CDH6, FYN, FKBP2, FAM126B, AKR1A1, IPCEF1, CYP4X1, MALL, KCNMA1, EVA1B, FES, MAGI1, CLIC2, ABCG8, BCL2, BBS1, AP4E1, CDH16, CADM1, ALDH3A2, IBTK, NCAM1, IGFLR1, HPS6, OR10R2, GRIN2D,
   i. Tissue protected: Pancreatic endocrine cells (Islets) STXBP1, CACNB2, SCGN, SLC25A17, STX1A, RTN1, SCG3, RAB3B, KCNQ4, MXRA7, PTPRN, PTH2R, MCOLN1, OBSCN, AATK, NECAB2, SLC17A6, ATP6V1D, SHISAL2B, SV2A, YRDC, TSPAN7, TBC1D3, SLC30A8, PRRG2, SLC25A25, PTPRN2, RUFY3, TEX261, SLC35G2, NAPB, DGCR2, CYP2W1, CDH6, GNAO1, FAM169A, ABCG8, FXYD2, ATP1A2, CADPS, ASIC5, BBS1, PAM, CADM1, GAD2, KCND1, IGFLR1, HPS6, OR10R2, MDGA2, DPP4,
   j. Tissue protected: Pancreatic exocrine cells UBE2J1, SELPLG, PCSK5, RAB17, ENPP1, SH3BP4, SLC19A3, RNF144B, IFI27, SYBU, STXBP3, MITD1, MXRA7, PTH2R, MCOLN1, LRRN2, OBSCN, AATK, VSIG10, YRDC, SLC16A14, TBC1D3, SYCN, SLCO2B1, SLC4A4, PKHD1, SCN7A, TMEM219, CRK, CSK, CA2, PRKCZ, DPEP1, CUZD1, GP2, KIRREL2, FA2H, ADCY10, BMPR1A, CDH6, AP4M1, FKBP2, AKR1A1, GPAT2, GPRC5B, FES, MAGI1, LEPROTL1, AQP1, ELOVL7, AP1S2, CRB3, ANPEP, CFTR, CLDN10, FXYD2, AP4E1, ASPHD1, PIK3AP1, CADM1, AQP12A, AQP12B, IBTK, MCUB, HPS6, OR10R2,
5. Cancer treated: head and neck cancer
   a. Tissue protected: Colon AXL, ZDHHC2, SLC30A7, SFT2D2, UGT2A3, TMEM237, TMEM74B, TNFSF14, TTC17, ATP8B2, CORO7, ACY3, CHST11, FIBCD1, GABARAPL2, SLC46A1, SCGN, ST3GAL6, ZGRF1, SLC47A1, SNX24, SMIM24, SRI, SLC26A2, NAAA, SFXN5, SOCS7, PEX1, PEMT, NDUFS8, SLC19A3, PARD3, PLPP1, RHOH, PIGR, PLEKHA3, TPCN1, TULP3, TJP3, WSCD1, TCIRG1, WHAMM, DLC1, PIGU, RAP1GAP, PSTPIP2, RAB3B, MFSD10, SEL1L3, TMEM45A, SERP1, SCARA3, SLCO4A1, SPPL3, STYK1, MAPKAP1, SLC9A3, IFI27, ITLN1, PTGDR, NECAP1, PLIN2, PEX10, ABCC3, OSBPL7, CD46, PCDH11X, GPR183, LRRC55, SLC24A3, MACF1, MGST2, MGAT4A, LRRN2, CNEP1R1, DENND5B, OBSCN, AATK, MEGF8, PNKD, SLC26A3, SERP2, TNFAIP8L3, SELENOT, SLC3A1, UGT2B17, VSIG10, TMEM236, VPS28, VPS35L, UGT2B28, SYTL4, SYTL3, TMEM64, TMEM253, TSPAN31, TMEM41A, TSPAN8, TNFRSF14, SYTL2, TMEM201, UGT2B15, MCUR1, PCDH1, SLC28A3, PTAFR, RAB27A, SYP, SUSD6, PIGZ, PTPRN2, SLC39A14, SLC6A12, SLC6A15, RAB31, TRPM4, VTI1B, TNFRSF10A, TMEM164, STK38L, MTG2, PIK3R5, IMMT, NAPB, RNF186, TMEM219, TMEM171, TAS2R38, TMEM39B, ADCK2, ATG2B, CA2, CCR10, CDHR5, PRKCZ, DPEP1, GSDMB, FKRP, GPC4, CX3CR1, DPY19L4, SLC48A1, CLEC16A, FCER1G, GOLPH3, DMXL2, ABCG2, ACSLS, CLCA1, GLP2R, CEACAM1, FPR2, FAR2, CHDH, ADGRF1, HTR1A, CLN6, CTNND1, DMTN, FLNC, FXYD6, ARL8B, AQP8, ELOVL1, CA4, SLC37A4, DPP10, HRH1, GNG11, DGKQ, GPR82, KCNMA1, IGSF11, CTDNEP1, EVA1B, GJA4, GALNT16, MAGI1, FPGS, MAN1C1, AP3M1, FER1L6, ANO8, BEST2, BRI3, BCL2, HTR4, B3GNT8, ELOVL7, CDHR2, DENND5A, ACE2, ANPEP, CNTNAP3, CDH17, ALG2, ATP11C, CLDND1, CYP20A1, GRAMD1A, ASIC5, B3GNT6, TMEM30B, ABHD12, AGPS, EBP, B3GNT7, HSD3B7, ALG14, CASD1, CHMP2B, ABCD3, B3GAT1, HHLA2, IBTK, SMPD4, SLC9A3R2, MPZL1, NCEH1, NBAS, GIPC1, MC4R, GFRA2, GLUL, MCUB, SLC2A10, SLC2A6, KIAA2013, NIPAL1, NIPAL4, APOOL, IL17RA, GPA33, DPY19L1, HIGD1A, LZTR1, MUC13, HPS6, MT-ND4, MFSD2A, NEU4, OR10R2, MUC4, PDLIM4, IL4R, PSKH1,
   b. Tissue protected: Lung TGM2, AXL, SLC30A7, SLC30A4, CHST11, ST3GAL6, TRIM13, RNF144A, PIGS, AGER, WHAMM, RAP1GAP, PTPRG, MFSD10, CD46, GPR183, PSD4, SLC6A15, VTI1B, TMEM164, STEAP1, STK38L, EHD4, FCER1G, CYB5A, SLC44A2, ACSL5, AOC3, CTXN2, AIFM2, LPAR6, AP3M1, CLIC2, AIG1, ELMO2, AQP4, CACNA2D2, ACE, AP3S1, CD63, CYP20A1, AP3S2, CACNA1C, CADM1, ABCD3, CAVIN2, IBTK, NCEH1, LAMP3, MC4R, NSMF, SLC34A2, PSKH1, LRRK2,
   c. Tissue protected: Skin TMEM243, ZDHHC2, TMEM237, CLEC10A, ACER3, CHST11, PERP, RNF145, TULP3, REEP2, ITGA9, PLIN2, ABCC3, GPR183, MGST2, F2R, OBSCN, AATK, PNKD, RMC1, TMEM260, ZP2, SYTL3, UBR4, VTI1B, TMEM164, TMEM171, DMXL2, ARL8A, ANO7, TESC, EPHA4, ATRAID, CTNND1, DMTN, ARL8B, CD99, ELOVL1, CAPNS2, ANKRD13A, GSDMA, FGFBP1, EVA1B, GALNT16, AP3M1, FADS3, B3GNT4, ADGRF4, ASPRV1, DSG3, COL13A1, AGPS, CNPPD1, ABCD3, MPZL1, NBAS, APOOL, MFSD2A, OR10R2, PSKH1, TMEM243, MGAT4C, TYR, TCIRG1, RAP1GAP, SEL1L3, TMEM45A, IFI27, ICAM3, LRRC55, MERTK, VPS28, DCT, ZC4H2, MCUR1, PMEL, TMEM219, B3GNT5, GPX8, FAR2, CYB561A3, FAT2, FAM210A, SLC37A4, BCL2, BBS1, CLEC2A, HSD3B7, GIPC1, ALOX12, SLC2A11, ,
   d. Tissue protected: Liver TECR, TNFSF14, APLNR, CLEC10A, ST3GAL6, TRIM13, ALPL, NAAA, RNF144A, SLC25A40, SLCO1B3, SLCO1B1, SLC25A43, SLC27A5, SLC19A3, PIGS, RHOH, TPCN1, PIGU, PDZK1, RDH16, TMEM45A, SCARA3, SPPL3, MAPKAP1, CD46, GPR183, LRRC55, MGST2, MERTK, LRRN2, DENND5B, SORD, SLC25A15, AATK, MEGF8, UGT2B4, YIPF1, TFR2, TMEM260, VPS35L, ZDHHC8, UGT1A6, TMEM64, TNS2, TBCD, UBR4, TMEM201, TSC2, PTAFR, SLC39A14, SLC6A12, SERINC1, VTI1B, TMEM164, TSPAN2, TMEM171, CA2, FCER1A, GPC4, EVA1A, GHR, DPY19L4, SLC48A1, CLEC16A, CYP2W1, CYP2D6, CYB5A, ACSL5, B3GNT5, CACNA1A, FYN, GAS2, FAR2, SLC2A2, HTR1A, TESC, CYP2A7, CYP1A2, CTNND1, FXYD6, CYP3A4, FAM210A, CYP2A6, SLC37A4, FMO3, CYP4V2, ABCB4, NINJ1, CYP2C8, DHCR24, CDH2, CTDNEP1, KMO, GJA4, CYP2C19, AQP9, ABCA8, AIG1, BRI3, UGCG, AP1S2, CDHR2, ANPEP, ENPEP, ASGR1, SLC31A1, ABCB11, CYP2A13, CLDND1, CYP20A1, CYP2E1, CTSL, ASIC5, ACAD11, TMEM30B, BBS1, CMTM8, EPB41L5, HSD11B1, CYTH2, EBP, ASGR2, CYP3A43, HSD3B7, CHMP2B, ABCD3, INTS2, CYP2C9, IBTK, GRB14, MPZL1, NBAS, LHFPL2, MFAP3L, GIPC1, GLUL, MTCH2, GK, SLC2A6, NIPAL4, APOOL, DPY19L1, HIGD1A, HPS6, POMGNT1, ABCC2, PSKH1, SLC10A1, NPC1L1,
   e. Tissue protected: Kidney (glomeruli) SLC30A7, TECR, ZAP70, CORO7, ACER3, ST3GAL6, MAPT, SLC45A1, RNF144A, PLPP1, WHAMM, KSR2, RAP1GAP, PTPRG, MRGPRF, SCARA3, TPSG1, PECAM1, PLA2R1, CD46, PTH2R, KCNAB1, PNPLA3, MACF1, F2R, MAP6, NPHS2, RMC1, TNS2, PSD4, TNFRSF10A, PTPRO, SREBF1, SLC35B1, TMEM171, VASN, PRKCZ, HYAL2, CX3CR1, EHD4, AIF1L, ADGRF1, CD34, DMTN, ENG, CD99, FAM210A, CR1, GPR82, GPRC5B, EVA1B, GJA4, GALNT16, FADS3, ANO8, AQP1, CRB3, ENPEP, EPB41L5, CACNA1C, SLC9A3R2, MPZL1, LIMS2, ITGA8, NIPAL4, LZTR1, NFXL1, HPS6, MFSD2A, NEU4, DYSF, PSKH1,
   f. Tissue protected: Kidney (tubules) TMEM243, VNN1, AXL, ZDHHC2, SLC30A7, SFT2D2, TRPM3, TMEM237, TMEM74B, TREH, TNFSF14, TTC17, ACER3, ACY3, FIBCD1, FCAMR, GABARAPL2, ST3GAL6, UBE2J1, ZGRF1, SLC47A1, MAPT, ALPL, SMIM24, SLC5A12, SLC5A2, SLC12A1, NAAA, RNF144A, SLC25A40, SFXN5, PEX1, RDX, PEMT, SLC6A19, SLC22A11, S1PR2, NDUFS8, SLC34A3, SLC19A3, PARD3, ARHGAP5, NEGR1, PTH1R, PDGFC, PIGS, NOXA1, PIGR, TPCN1, VPS26B, WSCD1, TCIRG1, WHAMM, PTPRD, PARVB, DLC1, PIGU, RAP1GAP, PDZK1, LIN7A, MRGPRF, SEL1L3, UCHL1, SERP1, SCARA3, RASA4, SLCO4A1, SPPL3, ITGA9, MAPKAP1, NECAP1, ABCC3, OSBPL7, CD46, GPR183, LRRC55, NAT8, PNPLA3, SLC24A3, PLCD4, MACF1, MGST2, MGAT4A, MERTK, LRRN2, CNEP1R1, DENND5B, SORD, SLC25A15, OBSCN, AATK, MEGF8, PNKD, UGT2B4, ATP6V1D, SLC22A6, SERP2, TMEM72, TNFAIP8L3, UGT3A1, XPNPEP2, SLC3A1, VSIG10, VPS28, ATP6V0A4, SLC7A7, TMEM80, TMEM260, VPS35L, ZDHHC8, ZP2, VAC14, UGT1A6, TMEM252, TMEM174, TMEM64, UBR4, TSPAN31, TSPAN8, TNFRSF14, SYTL2, TMEM201, TMPRSS2, TSC2, SLC28A1, MCUR1, SLC22A2, PCDH1, SLC28A3, SCAMP5, PTAFR, SLC22A8, SLC13A3, PIGZ, SLC39A14, SLC6A12, RUFY3, RAB31, SLC6A18, SLC13A2, TMEM213, TRPM4, VTI1B, TNFRSF10A, TMEM164, TMEM184A, SLC4A4, SLC25A23, TMEM178B, SREBF1, PIK3R5, PKHD1, PRRT3, IMMT, SLC35G2, OPRD1, NAPB, TSPAN2, TMEM219, TMEM171, VASN, TSPAN16, TMEM39B, ADCK2, CA2, CDHR5, ENPP6, GGT1, JAM3, DPEP1, ENPP3, CUBN, FKRP, FCER1A, GPC4, EVA1A, GHR, HYAL2, GSDMC, CX3CR1, DPY19L4, SLC48A1, CLEC16A, CPNE6, CYB5A, SLC44A2, DMXL2, AIF1L, GJA5, ACSL5, CDH6, FAM151A, B3GNT5, GAS2, SLC2A2, CHDH, HTR1A, TESC, CLN6, TM7SF2, ATRAID, CASR, FOLH1, CTNND1, DMTN, FXYD6, ARL8B, CNTFR, CLIC4, FAM210A, ELOVL1, ANKRD13A, SLC37A4, HRH1, GNG11, DGKQ, GPR82, KCNMA1, GPRC5B, IGSF11, CDH2, CTDNEP1, KMO, LRFN4, GJA4, GALNT16, MAGI1, AP3M1, BSND, ANO8, AQP6, AQP1, CDC42EP5, BCL2, MPP1, UGCG, AP1S2, CDHR2, ANK2, DENND5A, ANPEP, ENPEP, SLC7A9, CLDN10, SLC31A1, ALG2, SLC25A31, FXYD2, ACE, CMKLR1, ACKR3, CLDND1, CLDN2, CYP20A1, GRAMD1A, TMEM63B, ANXA13, BBS1, EPB41L3, AGPS, CACNA1C, CYP3A43, HSD3B7, CDH16, CHMP2B, CADM1, ABCD3, B3GAT1, INTS2, ABCC5, SMPD4, MOXD1, HAS3, MPZL1, NBAS, LHFPL2, GIPC1, MC4R, GFRA2, KCNK5, MCUB, MTCH2, SLC2A10, GK, SLC2A6, KIAA2013, NIPAL1, APOOL, FMO1, GAL3ST1, DPY19L1, HIGD1A, LRP2, LZTR1, ALOX12, HPS6, MT-ND 4, CHL1, POMGNT1, SLC2A11, MFSD2A, OR10R2, GRIN2D, NSMF, ABCC2, PDLIM4, DYSF, PSKH1, LRRK2,
   g. Tissue protected: Heart (cardiomyocytes) TGM2, TCTN2, SLC30A7, SLC30A4, SLC27A6, TECR, TTC17, APLNR, CAP2, GABARAPL2, MGAT4C, RASL10B, ST3GAL6, SLC14A1, TMEM106B, SNX24, PITPNM1, RNF144A, SFXN5, SLC4A10, SGCB, PEMT, NDUFS8, NTRK3, RNF145, SLC19A3, NEGR1, BVES, TPCN1, TCIRG1, PARVB, POPDC2, P2RY6, PLN, RAP1GAP, MFSD10, SERP1, SCARA3, SGCG, SLCO4A1, SRL, MAPKAP1, GPR183, LRRC55, MACF1, MERTK, CNEP1R1, DENND5B, DMPK, SLC25A15, OBSCN, AATK, MEGF8, PNKD, SERP2, SIPA1, YRDC, TMEM143, ZP2, VAC14, TMEM64, UBR4, TSPAN31, PTPRB, TSC2, SLC25A25, RAB11FIP4, SUSD6, SLC39A14, SLC6A12, SERINC1, VTI1B, TNFRSF10A, TMEM164, SLCO2B1, STK38L, PPM1L, TMEM178B, ST8SIA2, IMMT, TMEM219, TMEM171, VASN, TMEM39B, ATG2B, CCR10, CNTN3, DPY19L4, CLEC16A, CKMT2, CYP2W1, DMXL2, CDH6, CDH15, FNDC5, GNB5, RASGRP2, HTR1A, CAVIN4, SLC2A4, HHATL, FLNC, FXYD6, CHRNA1, CNTFR, CD248, ELOVL1, GPAT2, SLC37A4, CYP4V2, DGKQ, GPR82, CAMK2B, IGSF11, CDH2, CTDNEP1, EVA1B, LEPROTL1, AP3M1, ABHD16A, AP1S2, CDC42SE1, CDHR2, ANK2, DENND5A, CNTNAP3, SLC31A1, CSF3R, ATP11C, CAV3, CPT1B, AKAP6, ADGRG6, ADRB1, ACAD11, BBS1, BCL2L13, CNPPD1, CACNA1C, PAM, ABCD3, ABCC5, SMPD4, GRB14, MPZL1, NCEH1, MYZAP, NBAS, MFAP3L, NCAM1, GIPC1, GPR182, MC4R, MTCH2, SLC2A10, GPR15, MLIP, NIPAL4, APOOL, SLC16A10, DPY19L1, HIGD1A, NFXL1, MT-ND4, POMGNT1, ICA1, OR10R2, DES, DYSF, GPR63, PSKH1,
   h. Tissue protected: Thyroid SLC30A7, SLC30A4, SFT2D2, TMEM237, TMEM74B, TNFSF14, APLNR, FIBCD1, TMEM100, TMEM106B, VPS33B, RNF144A, AHCYL1, SFXN5, SCNN1D, PEMT, NTRK3, RNF145, SLC19A3, TPO, TPCN1, TULP3, TJP3, VPS26B, TCIRG1, RHBDD2, LMAN2, PIGU, RAP1GAP, SEL1L3, SERP1, SCARA3, MAPKAP1, IFI27, OSBPL7, CD46, GPR183, PSD3, LRRC55, SLC24A3, MACF1, MGAT4A, CNEP1R1, DENND5B, SORD, OBSCN, AATK, PNKD, SERP2, VPS28, TMEM260, VPS35L, SYTL4, SYTL3, TBCD, UBR4, TMEM41A, ZC4H2, TSC2, PTAFR, SLC26A4, RAB31, VTI1B, TMEM164, RNFT2, STK38L, PPM1L, SLC25A23, SREBF1, PIK3R5, IMMT, TMEM39B, IYD, DUOX1, GPC4, GHR, DPY19L4, CLEC16A, FCER1G, CYP2W1, ARL8A, CDH6, B3GNT5, FYN, FAR2, CLN6, EPHA4, CTNND1, FXYD6, ARL8B, CD99, ANKRD13A, IPCEF1, KCNMA1, LPAR6, CTDNEP1, EVA1B, GJA4, GALNT16, MAGI1, AP3M1, CLIC2, BCL2, AAK1, ALG2, ACKR3, CD63, CD300A, CYP20A1, ARRDC1, BBS1, CMTM8, AGPS, CDH16, CADM1, ABCD3, CAVIN2, IBTK, MPZL1, NBAS, LHFPL2, LRP1B, NCAM1, GIPC1, MC4R, GFRA2, GLUL, GK, IL17RA, HPS6, POMGNT1, ICA1, MFSD2A, OR10R2, GRIN2D, PDLIM4, PSKH1,
   i. Tissue protected: Pancreatic endocrine cells (Islets) ZDHHC2, TTC17, SCGN, STX1A, NAAA, RNF144A, RTN1, SCG3, PARD3, PTGDR2, RAP1GAP, RAB3B, UCHL1, SERP1, PTPRN, CD46, PTH2R, GPR183, LRRC55, MGST2, MGAT4A, OBSCN, AATK, NECAB2, PNKD, SLC17A6, ATP6V1D, SHISAL2B, SV2A, YRDC, ZDHHC8, VAC14, TSPAN7, TMEM64, UBR4, SLC30A8, SLC25A25, SYP, PTPRN2, RUFY3, RAB31, VTI1B, PPM1L, IMMT, SLC35G2, NAPB, ATG2B, GPC4, DGCR2, CYP2W1, DMXL2, CDH6, CLCN5, B3GNT5, FAR2, GNAO1, TESC, FAM169A, CASR, CD99, CNTFR, CDH2, CTDNEP1, UGCG, ENO2, ELMO2, ATP9A, CLU, ALG2, FXYD2, ATP1A2, CLDND1, CYP20A1, ARRDC1, ASIC5, BBS1, EPB41L3, PAM, CADM1, GAD2, ABCD3, ALOX5, NBAS, GIPC1, MC4R, APOOL, HPS6, MT-ND4, OR10R2, PDLIM4, PSKH1,
   j. Tissue protected: Pancreatic exocrine cells ZDHHC2, TMEM237, TNFSF14, CHRM3, GABARAPL2, RASL10B, UBE2J1, ZGRF1, STIMATE, NAAA, AHCYL1, SFXN5, SLC4A10, PEMT, TMEM97, SLC19A3, PARD3, PIGS, NOXA1, PLEKHA3, TJP3, TCIRG1, RAP1GAP, MFSD10, SEL1L3, SCARA3, IFI27, ABCC3, PTH2R, LRRC55, SLC24A3, MGST2, MGAT4A, MERTK, LRRN2, OBSCN, AATK, MEGF8, PNKD, SELENOT, SLC3A1, VSIG10, YRDC, TMEM80, ZDHHC8, VAC14, SYTL4, TMEM128, TSPAN31, TSPAN8, PTPRB, SYTL2, TMPRSS2, ZC4H2, SYCN, SLC28A3, SUSD6, SLC39A14, SERINC1, VTI1B, SLCO2B1, MTG2, SLC4A4, PKHD1, IMMT, TMEM219, TAS2R38, CA2, PRKCZ, DPEP1, CUZD1, GPC4, GP2, KIRREL2, DPY19L4, CLEC16A, FCER1G, GOLPH3, DMXL2, CDH6, B3GNT5, FAR2, TESC, CLN6, CTNND1, ARL8B, AQP8, CNTFR, FAM210A, GPAT2, CYP4V2, DGKQ, GPRC5B, LPAR6, CTDNEP1, MAGI1, LEPROTL1, AP3M1, AQP1, KDELR3, ELOVL7, AP1S2, CRB3, ACE2, ANPEP, CFTR, CLDN10, ALG2, FXYD2, ACKR3, CLDND1, CD300A, CYP20A1, ARRDC1, COL13A1, CMTM8, PIK3AP1, B3GNT7, CADM1, ABCD3, ARFGAP3, AQP12A, AQP12B, IBTK, SMPD4, MOXD1, GRB14, MPZL1, NBAS, GIPC1, MC4R, MCUB, SLC2A10, SLC2A6, APOOL, SLC16A10, IL17RA, HPS6, MYOC, MT-ND4, POMGNT1, SLC2A11, OR10R2, SLC16A7, PDLIM4, PSKH1,
6. Cancer treated: colorectal cancer
   a. Tissue protected: Colon TMEM138, SCGN, ST3GAL6, TMEM263, SOCS7, SEMA5A, SLC26A1, WHAMM, RAB27B, DLC1, RAB3B, ADIPOR2, MAPKAP1, ITLN1, TAOK2, LRRC55, OBSCN, SLC26A3, TUB, TMEM253, SYP, SLC39A2, SLC6A12, TMEM171, CA2, EFHD1, FKRP, ABCG1, ABCG2, CD177, GLP2R, GATM, ADGRA3, IGSF11, LRRC37B, ANKFY1, ANPEP, CNTNAP3, BIK, DCXR, CASD1, B3GAT1, SLC9A3R2, KCNN3, GFRA2, LZTR1, OR10R2,
   b. Tissue protected: Lung SLC30A4, ST3GAL6, TRIM13, VIPR2, SLC22A15, SLC25A17, RNF144A, AGER, TMEM43, WHAMM, RECK, PTPRG, EHD2, PRRG2, PSD4, STEAP1, EHD1, EHD4, CCR7, CYB5A, CAVIN1, GPC5, AOC3, CDH11, DCBLD1, CLIC2, AIG1, FCAR, ELMO2, CAV1, AQP4, CYB561D1, CACNA2D2, ACE, ANXA1, CAVIN2, LAMP3, EHD3, SLC34A2, LRRK2,
   c. Tissue protected: Skin TMEM243, TMEM138, CLEC10A, ACER3, VIPR2, TGFBR3, TMEM263, ENPP1, SEMA5A, TMEM43, MTMR2, REEP2, LYPD3, KRT1, F2R, PLA2G4E, OBSCN, RMC1, TUB, ZP2, THBD, TMEM171, CLCA2, GJB4, FA2H, CAVIN1, CD177, ANO7, EPHA4, GATM, ATRAID, ADGRA3, CD99, GJA1, CAPNS2, GSDMA, B3GNT4, DSG1, ADGRF4, CAV1, ANXA1, ASPRV1, DSG3, DCXR, CNPPD1, TACR1, OR10R2, TMEM243, TYR, SLC26A1, ADIPOR2, LRRC55, DCT, RHCG, RYK, PMEL, GPC5, B3GNT5, GPX8, CYB561A3, FAT2, FAM210A, BST1, CYB561D1, ALOX12, SLC2A11, ,
   d. Tissue protected: Liver TMEM138, CLEC10A, ST3GAL6, TRIM13, SYT13, ALPL, ENPP1, RNF144A, SLC25A40, SLC27A2, SLCO1B3, SLCO1B1, SLC27A5, RDH16, KCNQ4, MAPKAP1, TAOK2, LRRC55, SLC25A15, UGT2B4, YIPF1, TFR2, TUB, TNS2, SLC39A2, SLC6A12, TSPAN2, TMEM171, CA2, EVA1A, CYP2D6, CYB5A, ABCG1, CD177, B3GNT5, CACNA1A, GAS2, SLC2A2, CYP2A7, GATM, EPHX1, CYP1A2, CYP3A4, FAM210A, CYP2A6, CYP2B6, FMO3, NINJ1, CYP2C8, CDH2, FES, KMO, CYP2C19, BCL2L10, AQP9, ABCA8, AIG1, ANKFY1, ANPEP, ASGR1, ABCB11, CYP2A13, CTSL, DCXR, CMTM8, ASGR2, CYP2C9, MFAP3L, GK, IGFLR1, GAA, NPC1L1,
   e. Tissue protected: Kidney (glomeruli) TMEM138, ZDHHC6, ACER3, ST3GAL6, VIPR2, KDR, MAPT, SYT13, TGFBR3, SLC45A1, RNF144A, PODXL, KIRREL1, WHAMM, ITGB1, KSR2, RECK, PTPRG, MRGPRF, PECAM1, SCUBE1, PLA2R1, PTH2R, F2R, EHD2, NPHS2, RMC1, TNS2, PRRG2, SNCA, PSD4, RYK, PTPRO, SLC35B1, TMEM171, EHD1, EHD4, AIF1L, CAVIN1, DCBLD1, CD34, ENG, CD99, GJA1, FAM210A, CR1, GPRC5B, CAV1, BIK, ANXA1, COL6A2, SLC9A3R2, LIMS2, ITGA8, EHD3, LZTR1, KIAA0040, DYSF,
   f. Tissue protected: Kidney (tubules) TMEM243, TRPM3, ZDHHC6, TREH, ACER3, ST3GAL6, UBE2J1, SELPLG, MAPT, ALPL, TMEM263, ENPP1, SLC5A12, SLC5A2, SLC12A1, RNF144A, SLC25A40, SLC27A2, SLC6A19, S1PR2, SLC34A3, SEMA5A, SLC26A1, NEGR1, PTH1R, RHBG, WHAMM, PTPRD, RAB27B, DLC1, KCNH8, KCNJ1, LIN7A, MRGPRF, UCHL1, UPK3BL2, TMEM132E, KCNQ4, MAPKAP1, LRRC55, NAT8, KIRREL3, SLC25A15, OBSCN, UGT2B4, ATP6V1D, SLC22A6, TMEM72, UGT3A1, ATP6V0A4, SLC7A7, TMEM80, ZP2, QTRT1, TMEM252, TMEM174, RHCG, TMEM255B, UPK3BL1, SLC28A1, SLC22A2, SCAMP5, SLC22A8, SLC39A2, SLC6A12, RUFY3, SLC6A18, SLC13A2, TMEM213, SLC4A4, SLC23A1, RYK, SLC35G2, OPRD1, TSPAN2, TMEM171, TSPAN16, CA2, ENPP6, EFHD1, CUBN, FKRP, EVA1A, GSDMC, CPNE6, CYB5A, ABCG1, AIF1L, GJA5, GPC5, CDH6, FAM151A, B3GNT5, GAS2, SLC2A2, ABHD17C, CALCR, CREB3L2, GATM, ATRAID, CASR, FOLH1, CLIC4, GJA1, FAM210A, GPRC5B, IGSF11, CDH2, FES, KMO, LRFN4, BSND, BCL2L10, CDC42EP5, ANKFY1, SH3BP5, ANPEP, CLDN10, FXYD2, ACE, COX7B, TMEM63B, DCXR, EPB41L3, ATP12A, CDH16, B3GAT1, NDUFS1, GFRA2, KCNK5, GK, FMO1, LRP2, LZTR1, ALOX12, CHL1, NXPE2, SLC2A11, OR10R2, MDGA2, DYSF, LRRK2,
   g. Tissue protected: Heart (cardiomyocytes) TCTN2, SLC30A4, SLC27A6, TMEM138, UCP3, CAP2, CD36, RASL10B, ST3GAL6, VIPR2, SLC22A15, TRPV4, SLC25A17, PITPNM1, RNF144A, SGCB, NEGR1, BVES, TMEM43, P2RY6, PLN, ADIPOR2, SGCG, SRL, KCNQ4, MAPKAP1, LANCL2, LRRC55, DMPK, SLC25A15, OBSCN, YRDC, ZP2, PTPRB, PRRG2, SLC6A12, SLCO2B1, TEX261, TMEM171, CDH6, CDH15, DCBLD1, FNDC5, HHATL, FAM168B, CHRNA1, ADGRA3, CD248, GJA1, GPAT2, IGSF11, LRRC37B, CDH2, FES, LEPROTL1, ANKFY1, DMD, CDC42SE1, CNTNAP3, CAV1, BIK, CPT1B, COX7B, CDH13, ADRB1, CNPPD1, MYZAP, MFAP3L, NCAM1, NDUFS1, GPR15, IGFLR1, OR10R2, DES, DYSF,
   h. Tissue protected: Thyroid SLC30A4, ZDHHC6, TMEM100, VIPR2, VPS33B, SLC22A15, TRPV4, TGFBR3, TMEM263, ENPP1, RNF144A, NUS1, TPO, TMEM43, ADIPOR2, MAPKAP1, LANCL2, LRRC55, OBSCN, TUB, PRRG2, SLC39A2, RYK, IYD, DUOX1, ABCG1, CAVIN1, CDH6, B3GNT5, EPHA4, CD99, GJA1, IPCEF1, FES, CLIC2, AAK1, CAV1, BIK, ARRDC1, DCXR, CMTM8, CDH16, CAVIN2, MTNR1B, NCAM1, GFRA2, GK, IGFLR1, OR10R2, GAA,
   i. Tissue protected: Pancreatic endocrine cells (Islets) SCGN, TRPV4, SLC25A17, TGFBR3, STX1A, RNF144A, RTN1, SCG3, PTGDR2, RAB3B, UCHL1, KCNQ4, TAOK2, PTPRN, PTH2R, LRRC55, OBSCN, NECAB2, SLC17A6, ATP6V1D, SV2A, YRDC, TSPAN7, SLC30A8, PRRG2, TMEM255B, SYP, SLC39A2, RUFY3, TEX261, SLC35G2, DGCR2, CDH6, B3GNT5, GNAO1, FAM169A, CASR, CD99, CD82, CDH2, DGKZ, ELMO2, FXYD2, CADPS, ARRDC1, CFC1, EPB41L3, GAD2, IGFLR1, OR10R2, MDGA2, GAA,
   j. Tissue protected: Pancreatic exocrine cells CHRM3, RASL10B, UBE2J1, SELPLG, TMEM263, ENPP1, TMEM97, RAB27B, ADIPOR2, UPK3BL2, PTH2R, LRRC55, OBSCN, YRDC, TMEM80, TMEM128, PTPRB, TMEM255B, SYCN, SLC39A2, SLCO2B1, SLC4A4, RYK, CA2, CUZD1, KIRREL2, FA2H, CDH6, B3GNT5, GATM, EPHX1, FAM210A, GPAT2, GPRC5B, FES, L EPROTL1, ANPEP, CLDN10, FXYD2, BIK, ARRDC1, CMTM8, PIK3AP1, ARFGAP3, AQP12A, AQP12B, SLC2A11, OR10R2, GAA,
7. Cancer treated: liver cancer
   a. Tissue protected: Colon ZDHHC2, SCGN, SLC26A2, GOLGA8B, PILRA, WHAMM, DLC1, PLCB3, RNF215, PSTPIP2, RAB3B, ITLN1, PTGDR, OSBPL7, LRRC55, PLPP7, MAST2, OBSCN, SLC3A1, VSIG10, VSTM5, TMEM69, TMEM41A, UNC13B, ARHGAP17, RAB27A, SYP, PTPRN2, SNX6, TRAF3IP3, VTCN1, ATG2B, C2CD2L, DPEP1, GPBAR1, PHKA1, CLCA1, FAM126B, GOLGA8A, CARMIL2, EPCAM, FER1L6, BEST2, GAL3ST2, B3GNT6, ABHD12, C12orf66, ALG14, CASD1, B3GAT1, IBTK, IFFO1, MC4R, MARCKS, MCUB, GNG12, LZTR1, MUC13, NEU4, OR10R2, NDST2,
   b. Tissue protected: Lung SLC30A4, TRIM13, SLC22A15, SLC6A14, WHAMM, PTPRG, STX5, MXRA7, EHD2, PSD4, RNF103, STEAP1, PHLPP2, EHD1, GPBAR1, EHD4, CCR7, GPC5, CLIC2, AQP4, ACE, AQP3, CACNA1C, IBTK, LAMP3, MC4R, MARCKS, EHD3, NSMF,
   c. Tissue protected: Skin TMEM243, ZDHHC2, ACER3, PERP, RXFP1, TGFBR3, SLC5A6, RNF145, MTMR2, REEP2, SLC10A6, LYPD3, PLA2G4E, SLC8A1, OBSCN, RMC1, ZP2, TMEM69, TRAF3IP3, C2CD2L, CLCA2, IGSF8, FA2H, PHKA1, DSP, ANO7, FAM126B, CAPNS2, ANKRD13A, GSDMA, B3GNT4, DSG1, ASPRV1, DSG3, AQP3, CNPPD1, MARCKS, TACR1, OR10R2, TMEM243, MS4A8, MGAT4C, SLC6A14, TYR, SLC25A33, GOLGA8B, LRRC55, PLPP7, XKR9, DCT, S100A8, PMEL, GPC5, GPX8, FAT2, GOLGA8A, CARMIL2, HCN1, CLEC2A, ,
   d. Tissue protected: Liver TRIM13, SLC25A40, SLCO1B3, SLCO1B1, SLC25A33, STX5, LRRC55, SLC8A1, UNC13B, FYN, NINJ1, IBTK,
   e. Tissue protected: Kidney (glomeruli) ZDHHC6, ACER3, KDR, TGFBR3, SLC45A1, PODXL, TRPV6, WHAMM, RNF215, PTPRG, TPSG1, STX5, PLA2R1, PTH2R, PNPLA3, MAST2, MAP6, EHD2, NPHS2, RMC1, VSTM5, ARHGAP17, SNCA, PSD4, PTPRO, SREBF1, EHD1, EHD4, AIF1L, CD34, EVI2A, ENG, CR1, AKAP12, COL6A2, CACNA1C, LIMS2, ITGA8, EHD3, LZTR1, NFXL1, KIAA0040, NEU4,
   f. Tissue protected: Kidney (tubules) TMEM243, ZDHHC2, ZDHHC6, TREH, ACER3, UBE2J1, SELPLG, SLC5A12, SLC12A1, SLC25A40, S1PR2, SLC34A3, SLC25A33, MGAM, PTH1R, PDGFC, RHBG, WHAMM, DLC1, RNF215, KCNH8, UCHL1, MGAT4B, UPK3BL2, RASA4, STX5, TMEM52B, OSBPL7, LRRC55, PNPLA3, SLC8A1, OBSCN, SLC22A6, TMEM72, UGT3A1, SLC3A1, XKR9, VSIG10, ATP6V0A4, VSTM5, ZP2, QTRT1, TMEM252, TMEM174, UNC13B, UPK3BL1, ARHGAP17, SLC28A1, SCAMP5, SLC22A8, SNX6, SLC6A18, SLC13A2, TMEM184A, SLC4A4, SREBF1, SLC35G2, VTCN1, TSPAN16, ENPP6, JAM3, DPEP1, ENPP3, CUBN, GSDMC, PHKA1, AIF1L, GPC5, ADCY7, CDH6, FAM126B, CASR, FOLH1, CLIC4, ANKRD13A, CARMIL2, EPCAM, BSND, AQP6, ANK2, SH3BP5, SLC7A9, ACE, AQP3, EPB41L3, C12orf66, ATP12A, CACNA1C, CDH16, B3GAT1, ABCC5, IFFO1, MC4R, MARCKS, MCUB, GNG12, FMO1, LRP2, LZTR1, CHL1, OR10R2, NSMF, NDST2,
   g. Tissue protected: Heart (cardiomyocytes) SLC30A4, UCP3, MGAT4C, RASL10B, SLC14A1, SLC22A15, TLN2, SGCB, SLC25A33, RNF145, POPDC2, PLN, SGCG, SRL, MXRA7, PPP1R3A, LANCL2, LRRC55, PLPP7, SLC8A1, DMPK, OBSCN, YRDC, ZP2, ARHGAP17, ATG2B, PHKA1, DSP, ADCY7, CDH6, CDH15, FNDC5, FAM126B, RASGRP2, SLC2A4, HHATL, FAM168B, CHRNA1, CD248, GPAT2, CAMK2B, ABHD16A, CDC42SE1, ANK2, ATP1A3, CPT1B, CDH13, AKAP6, BCL2L13, CNPPD1, CACNA1C, PAM, ABCC5, MYZAP, NCAM1, GPR182, MC4R, GPR15, MLIP, SLC16A10, NFXL1, OR10R2, DES,
   h. Tissue protected: Thyroid SLC30A4, ZDHHC6, VPS33B, SLC22A15, TGFBR3, SLC5A5, SLC25A33, RNF145, TPO, TRPV6, RHBDD2, RNF215, MXRA7, OSBPL7, LANCL2, PSD3, LRRC55, SLC8A1, OBSCN, TMEM41A, UNC13B, ARHGAP17, RNFT2, SREBF1, IYD, DUOX1, CDH6, FYN, FAM126B, ANKRD13A, CARMIL2, HCN1, IPCEF1, EPCAM, CLIC2, AAK1, CDH16, IBTK, MTNR1B, NCAM1, MC4R, GNG12, OR10R2,
   i. Tissue protected: Pancreatic endocrine cells (Islets) ZDHHC2, SCGN, TGFBR3, STX1A, RTN1, SCG3, RNF215, RAB3B, UCHL1, MXRA7, PTPRN, PTH2R, LRRC55, MAST2, SLC8A1, OBSCN, NECAB2, SLC17A6, XKR9, SV2A, YRDC, TMEM69, TSPAN7, SLC30A8, RNF103, SYP, PTPRN2, SLC35G2, ATG2B, C2CD2L, DGCR2, PHKA1, CDH6, GNAO1, CASR, DGKZ, ENO2, ATP1A2, CADPS, EPB41L3, PAM, GAD2, MC4R, MARCKS, OR10R2,
   j. Tissue protected: Pancreatic exocrine cells ZDHHC2, RASL10B, UBE2J1, SELPLG, TMEM97, RNF215, UPK3BL2, STX5, MXRA7, PTH2R, LRRC55, MAST2, OBSCN, SLC3A1, VSIG10, YRDC, SYCN, SLC4A4, DPEP1, CUZD1, KIRREL2, FA2H, ADCY7, CDH6, EVI2A, GPAT2, KDELR3, AQP12A, AQP12B, IBTK, IFFO1, MC4R, MARCKS, MCUB, SLC16A10, OR10R2,
8. Cancer treated: stomach cancer
   a. Tissue protected: Colon UGT2A3, TMEM138, SCGN, TMEM245, SLC26A2, TEX2, SH3BP4, SCD5, SLC26A1, LDLR, PILRA, DLC1, RRAD, PIGU, RAB3B, STYK1, PTGDR, SNX1, SMPD2, OSBPL7, LRRC55, PALM, PLPP7, MSRA, OBSCN, MFN2, SLC26A3, SLC3A1, UGT2B17, VSIG10, TMEM236, VSTM5, UTRN, UGT2B28, TMEM69, SYTL3, TMEM41A, ZFYVE9, UGT2B15, SYP, SLC6A12, SNX6, TMEM204, ARHGEF12, ATG2B, C2CD2L, FMO5, ABCG2, CD177, CLCA1, GLP2R, FKBP2, ADGRF1, FAM126B, HTR1A, FGFRL1, GABARAPL1, FMNL3, CARMIL2, GBP2, LRRC37B, BEST2, ABCG8, BCL2, GAL3ST2, CLN5, B3GALT5, CDHR2, ACE2, CNTNAP3, CIB1, ASIC5, ABHD12, ALG14, CASD1, B3GAT1, IBTK, SLC9A3R2, SLC16A6, GFRA2, MCUB, JAML, NIPAL1, LZTR1, NEU4, OR10R2,
   b. Tissue protected: Lung TGM2, SLC30A4, TRIM13, TMEM245, SLC22A15, SLC25A17, SLC6A14, RNF144A, AGER, RECK, PTPRG, SNX1, GNAI3, PALM, EHD2, UTRN, PSD4, PHLPP2, GNAI1, EHD1, EHD4, CCR7, SLC44A2, AOC3, GABARAPL1, GBP2, AIFM2, CLIC2, AIG1, CAV1, AQP4, CACNA2D2, ACE, AP3S1, AP3S2, CACNA1C, DAPP1, CAVIN2, IBTK, LAMP3, EHD3, PI4KA, SLC34A2, ICAM1, LRRK2,
   c. Tissue protected: Skin TMEM243, TMEM138, CACNB2, CLEC10A, PERP, RXFP1, TGFBR3, RNF145, MTMR2, REEP2, TMEM134, ITGA9, SLC10A6, SNX1, GNAI3, PALM, LYPD3, KRT1, F2R, PLA2G4E, OBSCN, RMC1, ZP2, TMEM69, SYTL3, THBD, TACSTD2, ARHGEF12, C2CD2L, CLCA2, GNAI1, FA2H, CD177, ANO7, FAM126B, ATRAID, CD99, FMNL3, CAPNS2, ANKRD13A, GBP2, DSC3, B3GNT4, DSG1, ABCG8, ADGRF4, CLN5, CAV1, ASPRV1, CIB1, ORAI1, DSG3, CNPPD1, FGFR3, TACR1, OR10R2, TMEM243, MGAT4C, SLC6A14, SH3BP4, SLC26A1, ICAM3, LRRC55, PLPP7, DCT, RHCG, S100A8, PMEL, B3GNT5, FAT2, FAM210A, CARMIL2, MALL, CLEC2B, BCL2, BST1, CLEC2A, ALOX12, SLC2A11, ,
   d. Tissue protected: Liver TMEM138, TECR, CACNB2, CLEC10A, TRIM13, TMEM245, SYT13, ALPL, RNF144A, SLC27A2, SLCO1B3, SLCO1B1, SLC27A5, SERINC3, LDLR, RRAD, PIGU, PDZK1, RDH16, KCNQ4, SMPD2, NDUFAF5, LRRC55, SORD, SPRED1, UGT2B4, YIPF1, TFR2, ZDHHC8, TNS2, TBCD, SLC6A12, TMEM204, TSPAN2, FMO5, CYP2W1, CYP2D6, CD177, B3GNT5, FYN, GAS2, SLC2A2, HTR1A, CYP2A7, FGFRL1, EPHX1, CYP1A2, CYP3A4, FAM210A, FMNL3, CYP2A6, FMO3, GBP2, ABCB4, NINJ1, CYP2C8, CDH2, CLEC2B, KMO, CYP2C19, BCL2L10, AQP9, ABCA8, ABCG8, AIG1, CLN5, CDHR2, ASGR1, ABCB11, CYP2A13, CTSL, ASIC5, ASPHD1, HSD11B1, ASGR2, DAPP1, INTS2, CYP2C9, IBTK, SLC16A6, MFAP3L, POMGNT1, ABCC2, GAA, NPC1L1,
   e. Tissue protected: Kidney (glomeruli) TMEM138, TECR, LRIG1, TMEM245, KDR, SYT13, TGFBR3, SLC45A1, RNF144A, KIRREL1, TSPAN33, TRPV6, KSR2, RECK, PTPRG, MRGPRF, TPSG1, SCUBE1, SNX1, PLA2R1, PTH2R, F2R, MAP6, MRC2, EHD2, NPHS2, RMC1, VSTM5, UTRN, TNS2, SNCA, PSD4, PTPRO, SLC35B1, ARHGEF12, EHD1, EHD4, ADGRF1, CD34, EVI2A, ENG, CD99, FAM210A, FMNL3, CR1, GBP2, CLEC2B, ABCG8, CAV1, CACNA1C, ABCC1, SLC9A3R2, LIMS2, ITGA8, EHD3, LZTR1, KIAA0040, PI4KA, NEU4, ICAM1, DYSF,
   f. Tissue protected: Kidney (tubules) TMEM243, TRPM3, TREH, ARMCX2, CACNB2, STRA6, TMEM245, TM4SF18, UBE2J1, SELPLG, ALPL, SLC5A12, SLC5A2, TEX2, SLC12A1, RNF144A, SLC27A2, SCD5, RDX, SLC6A19, S1PR2, SLC34A3, SERINC3, SLC26A1, NEGR1, PTH1R, LDLR, RHBG, DLC1, RRAD, PIGU, PDZK1, KCNH8, KCNJ1, LIN7A, MRGPRF, UCHL1, MGAT4B, SLC34A1, UPK3BL2, PRICKLE1, TMEM52B, ITGA9, KCNQ4, SNX1, SMPD2, NDUFAF5, OSBPL7, GNAI3, LRRC55, NAT8, PALM, MSRA, KIRREL3, SORD, MPP5, OBSCN, MFN2, UGT2B4, ATP6V1D, SLC22A6, TMEM72, UGT3A1, XPNPEP2, SLC3A1, VSIG10, ATP6V0A4, VSTM5, UTRN, ZDHHC8, ZP2, QTRT1, TMEM252, TMEM174, TTC7B, ZFYVE9, RHCG, UPK3BL1, SLC28A1, SLC22A2, SCAMP5, SLC22A8, SLC6A12, RUFY3, SNX6, SLC6A18, SLC13A2, TMEM213, SYT7, TMEM204, SLC4A4, SLC35G2, OPRD1, TSPAN2, TSPAN16, ARHGEF12, ENPP6, GGT1, GNAI1, JAM3, ENPP3, CUBN, GSDMC, CPNE6, SLC44A2, GJA5, ADCY7, B3GNT5, GAS2, FKBP2, SLC2A2, FAM126B, HTR1A, CALCR, CREB3L2, FGFRL1, GABARAPL1, ATRAID, CASR, FOLH1, CLIC4, FAM210A, ANKRD13A, CARMIL2, GBP2, CDH2, CLEC2B, KMO, LRFN4, BSND, BCL2L10, AQP6, ABCG8, CDC42EP5, BCL2, MPP1, CLN5, CDHR2, ANK2, SH3BP5, SLC7A9, CLDN10, SLC25A31, FXYD2, ACE, CMKLR1, COX7B, TMEM63B, AP1S3, ASPHD1, EPB41L3, ATP12A, CACNA1C, CDH16, B3GAT1, INTS2, ABCC5, SLC16A6, GFRA2, KCND1, KCNK5, MCUB, NIPAL1, LRP2, LZTR1, ALOX12, CHL1, POMGNT1, PI4KA, SLC2A11, OR10R2, ABCC2, MDGA2, DYSF, LRRK2,
   g. Tissue protected: Heart (cardiomyocytes) TGM2, TCTN2, SLC30A4, SLC27A6, TMEM138, TECR, UCP3, ARMCX2, CAP2, CD36, MGAT4C, SLC14A1, SLC22A15, TRPV4, SLC25A17, SUSD3, TEX2, TLN2, RNF144A, SCD5, SGCB, RNF145, NEGR1, BVES, P2RX6, TSPAN33, POPDC2, P2RY6, PLN, RRAD, SGCG, SRL, KCNQ4, SMPD2, NDUFAF5, LANCL2, LRRC55, PLPP7, OBSCN, MFN2, YRDC, UTRN, ZP2, TBC1D3, SLC6A12, SLCO2B1, TMEM204, TEX261, ATG2B, CKMT2, CYP2W1, ADCY7, CDH15, GNB5, FAM126B, HTR1A, CAVIN4, SLC2A4, FGFRL1, HHATL, GABARAPL1, CHRNA1, CD248, FMNL3, GPAT2, DAG1, GBP2, CAMK2B, LRRC37B, CDH2, CLEC2B, LEPROTL1, ABHD16A, ABCG8, DMD, CDC42SE1, CDHR2, ANK2, CNTNAP3, CAV1, CSF3R, ATP2A2, CAV3, CPT1B, COX7B, CDH13, CIB1, ORAI1, AKAP6, ADGRG6, ADRB1, AP1S3, CNPPD1, CACNA1C, ABCC5, SLC16A6, MYZAP, MFAP3L, NCAM1, GPR182, KCND1, GPR15, MLIP, POMGNT1, PI4KA, OR10R2, PRKCA, DES, DYSF,
   h. Tissue protected: Thyroid SLC30A4, ARMCX2, TMEM100, TMEM245, VPS33B, SLC22A15, TRPV4, TGFBR3, TEX2, RNF144A, SCNN1D, RNF145, TPO, TSPAN33, TRPV6, RHBDD2, PIGU, SNX1, OSBPL7, LANCL2, PSD3, LRRC55, MSRA, SORD, OBSCN, MFN2, UTRN, SYTL3, TBC1D3, TBCD, TMEM41A, ZFYVE9, ARHGEF12, IYD, DUOX1, CYP2W1, B3GNT5, FYN, FKBP2, FAM126B, FGFRL1, GABARAPL1, CD99, FMNL3, ANKRD13A, CARMIL2, IPCEF1, CYP4X1, MALL, CLIC2, ABCG8, BCL2, CLN5, AAK1, CAV1, ARRDC1, AP1S3, CDH16, CAVIN2, IBTK, MTNR1B, NCAM1, GFRA2, POMGNT1, OR10R2, GAA,
   i. Tissue protected: Pancreatic endocrine cells (Islets) LRIG1, ARMCX2, CACNB2, SCGN, TRPV4, SLC25A17, TGFBR3, STX1A, RNF144A, RTN1, SCG3, PTGDR2, RAB3B, UCHL1, KCNQ4, SMPD2, PTPRN, PTH2R, GNAI3, LRRC55, OBSCN, MFN2, NECAB2, SLC17A6, ATP6V1D, SV2A, YRDC, ZDHHC8, TMEM69, TSPAN7, TBC1D3, ZFYVE9, SLC30A8, SYP, RUFY3, TEX261, SLC35G2, ARHGEF12, ATG2B, C2CD2L, GNAI1, DGCR2, CYP2W1, B3GNT5, GNAO1, FAM169A, FGFRL1, CASR, CD99, FMNL3, GBP2, CDH2, ABCG8, ENO2, FXYD2, ATP1A2, ARRDC1, ASIC5, CFC1, EPB41L3, GAD2, DAPP1, KCND1, PI4KA, OR10R2, LRRC24, MDGA2, GAA,
   j. Tissue protected: Pancreatic exocrine cells TMEM245, UBE2J1, SELPLG, STIMATE, TEX2, SH3BP4, TMEM97, TSPAN33, UPK3BL2, SNX1, SMPD2, PTH2R, LRRC55, MSRA, OBSCN, SPRED1, SLC3A1, VSIG10, YRDC, UTRN, ZDHHC8, TMEM128, TBC1D3, SYCN, SLCO2B1, TMEM204, SLC4A4, ARHGEF12, CUZD1, KIRREL2, FA2H, ADCY7, B3GNT5, FKBP2, EVI2A, FGFRL1, EPHX1, FAM210A, GPAT2, GBP2, LEPROTL1, CLN5, ACE2, CFTR, CLDN10, FXYD2, ARRDC1, ASPHD1, DAPP1, IBTK, MCUB, POMGNT1, SLC2A11, OR10R2, GAA,
9. Cancer treated: cervical cancer
   a. Tissue protected: Colon TMED6, SCN4B, TMEM138, SLC7A6, DST, GABARAPL2, SCGN, SLC47A1, PCSK5, SLC22A16, SNX24, SMIM24, SPIRE1, SIRPB1, PILRA, TPCN1, DLC1, PLCB3, SLC25A20, PVR, RNF144B, RAB3B, SEL1L3, SLC25A36, STYK1, MAPKAP1, SLC9A3, ITLN1, PTGDR, PCDHB15, SNX7, SLC11A1, MPC2, PEX10, MGLL, OSBPL7, PCDH11X, ITPR2, MFSD11, CNEP1R1, OBSCN, SLC3A1, VSIG10, TMEM236, TMEM41A, ZFYVE9, RINT1, TMEM86A, C2orf88, SPHK1, RAB27A, SYP, PIGZ, PTPRN2, SLC6A12, SNX6, VTI1B, SLC38A5, SUSD1, REEP4, TMEM39B, ACVR1B, EFHD1, DPEP1, FKRP, KCNMB3, CX3CR1, SLC48A1, CLEC16A, FCER1G, CLCA1, ADGRF1, CLN6, FGFRL1, DMTN, FXYD6, ADGRA3, CA4, HHAT, LRRC37B, MAGI1, MAN1C1, AP3M1, CDK14, GAL3ST2, APOBR, ACE2, ANPEP, CNTNAP3, ADCK5, ASIC5, ARL4C, TMEM37, ABHD12, ATP8B1, EBP, ALG14, CASD1, B3GAT1, IBTK, SLC9A3R2, LFNG, KCNN3, GFRA2, KIAA2013, NIPAL1, IL17RA, GPA33, RPS6KC1, LZTR1, MUC13, MRAP, MT-ND4, NEU4, OR10R2, NDUFB9, NDST2,
   b. Tissue protected: Lung SLC30A4, TRIM13, SLC22A15, SLC25A17, RNF144A, AGER, PAG1, VAT1, IL10RB, RECK, RNF144B, PTPRG, STX5, MXRA7, PSD4, VTI1B, STEAP1, PHLPP2, FCER1G, CCR7, CYB5A, SLC44A2, AOC3, DCBLD1, AIFM2, AP3M1, FCAR, ELMO2, AQP4, CACNA2D2, ACE, CACNA1C, CAVIN2, IBTK, LAMP3, LFNG, LDLRAD2, PI4KA, NSMF, SLC34A2, LRRK2,
   c. Tissue protected: Skin SCN4B, TMEM138, CACNB2, ACER3, TGFBR3, ENPP1, SPIRE1, VAT1, REEP2, MGLL, F2R, OBSCN, RMC1, TMEM260, ZP2, TMEM86A, C2orf88, VTI1B, GJB4, FA2H, DSP, DMTN, ADGRA3, CD99, GSDMA, AP3M1, B3GNT4, DSG1, APOBR, ASPRV1, ATP8B1, RPS6KC1, OR10R2, SCN4B, TYR, SEL1L3, SLC11A1, MPC2, ITPR2, DCT, PMEL, A RHGAP21, REEP4, CYB561A3, FAT2, HCN1, MALL, BBS1, CLEC2A, ALOX12, ,
   d. Tissue protected: Liver TMEM138, APLNR, CACNB2, CCND1, TRIM13, SYT13, PCSK5, ALPL, SLC22A16, RAB17, ENPP1, SLC38A9, RNF144A, SLC25A40, SLC27A2, SLCO1B3, SPIRE1, SLCO1B1, SLC27A5, TPCN1, VAT1, SLC25A20, RNF144B, RDH16, SLC25A36, STX5, KCNQ4, MAPKAP1, MPC2, MGLL, ITPR2, MFSD11, MME, SLC25A15, UGT2B4, TFR2, TMEM260, TNS2, RINT1, SLC6A12, VTI1B, TSPAN2, ACVR1B, FCER1A, EVA1A, KCNMB3, SLC48A1, CLEC16A, CYP2D6, CYB5A, CACNA1A, FYN, GAS2, SLC2A2, CYP2A7, FGFRL1, CYP1A2, FXYD6, CYP3A4, CYP2A6, HHAT, KCNJ16, ABCB4, NINJ1, CYP2C8, CDH2, FES, CDK14, CYP2C19, BCL2L10, AQP9, ABCA8, AP1S2, ANPEP, ENPEP, ASGR1, ABCB11, CYP2A13, ASIC5, BBS1, HSD11B1, ATP8B1, EBP, ASGR2, CYP3A43, INTS2, CYP2C9, IBTK, LHFPL2, MFAP3L, GK, IGFLR1, MRAP, ABCC2, DPP4, SLC10A1, NPC1L1,
   e. Tissue protected: Kidney (glomeruli) TMEM138, DST, ACER3, MAPT, SYT13, TGFBR3, RAB17, SLC45A1, RNF144A, SPIRE1, KIRREL1, ITGB1, RECK, PVR, PTPRG, MRGPRF, TPSG1, STX5, SCUBE1, PLA2R1, PTH2R, ITPR2, PNPLA3, F2R, MME, NPHS2, RMC1, TNS2, SNCA, PSD4, PTPRO, SREBF1, VASN, HYAL2, CX3CR1, DCBLD1, ADGRF1, EVI2A, DMTN, ENG, CD99, CR1, AQP1, ENPEP, CACNA1C, SLC9A3R2, LIMS2, GPR153, ITGA8, LDLRAD2, LZTR1, NFXL1, NPHS1, KIAA0040, PI4KA, NEU4, DYSF, DPP4,
   f. Tissue protected: Kidney (tubules) VNN1, TTYH3, SCN4B, TRPM3, TREH, DST, CACNB2, ACER3, FCAMR, GABARAPL2, UBE2J1, SLC47A1, MAPT, PCSK5, ALPL, SLC22A16, RAB17, ENPP1, SMIM24, SLC5A12, SLC5A2, SLC12A1, RNF144A, SLC25A40, SLC27A2, SPIRE1, SLC6A19, S1PR2, SLC34A3, MGAM, ARHGAP5, NEGR1, PTH1R, TPCN1, VAT1, VHL, PTPRD, DLC1, SLC25A20, RNF144B, KCNH8, LIN7A, MRGPRF, SEL1L3, UCHL1, S LC25A36, STX5, KCNQ4, MAPKAP1, SNX7, MPC2, MGLL, OSBPL7, ITPR2, NAT8, PNPLA3, PLCD4, KIRREL3, MFSD11, CNEP1R1, MME, SLC25A15, OBSCN, UGT2B4, ATP6V1D, SLC22A6, TMEM72, XPNPEP2, SLC3A1, VSIG10, ATP6V0A4, SLC7A7, TMEM260, UST, ZP2, QTRT1, TMEM252, TMEM174, TTC7B, ZFYVE9, RINT1, TMPRSS2, SLC28A1, SLC22A2, SPHK1, SCAMP5, SLC22A8, PIGZ, SLC6A12, RUFY3, SNX6, SLC6A18, SLC13A2, TMEM213, VTI1B, SLC4A4, SLC25A23, SREBF1, ARHGAP21, REEP4, SLC35G2, OPRD1, TSPAN2, VASN, TSPAN16, TMEM39B, ENPP6, EFHD1, GGT1, DPEP1, ENPP3, CUBN, FKRP, FCER1A, EVA1A, GREB1, HYAL2, KCNMB3, CX3CR1, SLC48A1, CLEC16A, CPNE6, CYB5A, SLC44A2, GJA5, DAB2, GAS2, SLC2A2, CLN6, CREB3L2, FGFRL1, CASR, FOLH1, DMTN, FXYD6, CLIC4, HHAT, KCNJ16, CDH2, FES, MAGI1, AP3M1, CDK14, BSND, BCL2L10, AQP6, AQP1, CDC42EP5, MPP1, AP1S2, SH3BP5, ANPEP, ENPEP, SLC7A9, CLDN10, SLC25A31, FXYD2, ACE, ADCK5, CMKLR1, TMEM63B, ACVRL1, ARL4C, ANXA13, BBS1, TMEM37, EPB41L3, ATP8B1, ATP12A, ATG9A, CACNA1C, CYP3A43, B3GAT1, INTS2, ABCC5, LFNG, LHFPL2, NDUFS1, GFRA2, KCND1, KCNK5, GK, KIAA2013, NIPAL1, FMO1, LRP2, LZTR1, ALOX12, MRAP, MT-ND4, NXPE2, PI4KA, OR10R2, GRIN2D, NDUFB9, NSMF, ABCC2, DYSF, DPP4, LRRK2, NDST2,
   g. Tissue protected: Heart (cardiomyocytes) TTYH3, TCTN2, SLC30A4, SCN4B, TMEM138, SLC7A6, UCP3, DST, APLNR, GABARAPL2, RASL10B, SLC14A1, TMEM106B, SLC22A15, PCSK5, SLC25A17, SLC22A16, RAB17, SNX24, SUSD3, PITPNM1, RNF144A, SLC4A10, SIRPB1, SGCB, NEGR1, BVES, TPCN1, P2RY6, SLC25A20, PLN, PVR, RNF144B, SGCG, KCNQ4, MAPKAP1, SNX7, MPC2, MXRA7, PPP1R3A, LANCL2, ITPR2, MFSD11, CNEP1R1, DMPK, SLC25A15, OBSCN, UST, ZP2, RINT1, TMEM86A, C2orf88, SLC6A12, VTI1B, SLCO2B1, ST8SIA2, TEX261, ARHGAP21, VASN, TMEM39B, GREB1, CLEC16A, CKMT2, DSP, CACNA2D1, CDH15, DCBLD1, RASGRP2, CAVIN4, FGFRL1, HHATL, FXYD6, CHRNA1, ADGRA3, CD248, FXYD7, GPAT2, HHAT, CAMK2B, KCNJ16, LRRC37B, CDH2, FES, LEPROTL1, AP3M1, ABHD16A, DMD, AP1S2, CDC42SE1, CNTNAP3, CSF3R, ADCK5, CAV3, CPT1B, CDH13, AKAP6, ADRB1, ARL4C, BBS1, AP4E1, ATP8B1, BCL2L13, ATG9A, CACNA1C, PAM, ABCC5, MYZAP, LFNG, MFAP3L, NCAM1, GPR182, NDUFS1, KCND1, GPR15, IGFLR1, LDLRAD2, NFXL1, MRAP, MT-ND4, PI4KA, OR10R2, NDUFB9, DES, DYSF, GPR63,
   h. Tissue protected: Thyroid SLC30A4, SCN4B, APLNR, TMEM100, TMEM106B, VPS33B, SLC22A15, TGFBR3, RAB17, ENPP1, RNF144A, SCNN1D, TPO, TPCN1, RHBDD2, PVR, SEL1L3, MAPKAP1, SNX7, MPC2, MXRA7, OSBPL7, LANCL2, PSD3, MFSD11, CNEP1R1, OBSCN, TMEM260, UST, TMEM41A, ZFYVE9, TMEM86A, SLC26A4, VTI1B, SLC25A23, SUSD1, SREBF1, ARHGAP21, TMEM39B, IYD, CLEC16A, FCER1G, DAB2, FYN, CLN6, FGFRL1, FXYD6, CD99, HCN1, IPCEF1, MALL, KCNJ16, FES, MAGI1, AP3M1, CDK14, AAK1, ADCK5, ACVRL1, BBS1, AP4E1, ATP8B1, CAVIN2, IBTK, LFNG, LHFPL2, NCAM1, GFRA2, GK, IGFLR1, IL17RA, MRAP, OR10R2, GRIN2D,
   i. Tissue protected: Pancreatic endocrine cells (Islets) CACNB2, SCGN, SLC25A17, TGFBR3, STX1A, RNF144A, RTN1, SCG3, VAT1, VHL, PVR, RAB3B, UCHL1, KCNQ4, MPC2, MXRA7, PTPRN, PTH2R, MFSD11, OBSCN, NECAB2, SLC17A6, ATP6V1D, SV2A, TSPAN7, ZFYVE9, SLC30A8, SYP, PTPRN2, RUFY3, VTI1B, TEX261, SLC35G2, GREB1, DGCR2, GNAO1, FAM169A, FGFRL1, CASR, CD99, FKBP11, CDH2, ENO2, ELMO2, ATP9A, CLU, FXYD2, ADCK5, ATP1A2, CADPS, ASIC5, BBS1, EPB41L3, ATP8B1, PAM, GAD2, ALOX5, LFNG, KCND1, IGFLR1, LDLRAD2, MT-ND4, PI4KA, OR10R2, LRRC24, DPP4,
   j. Tissue protected: Pancreatic exocrine cells TMED6, GABARAPL2, RASL10B, UBE2J1, PCSK5, SLC22A16, RAB17, ENPP1, STIMATE, SLC4A10, SPIRE1, TMEM97, VAT1, VHL, SLC25A20, RNF144B, SEL1L3, SLC25A36, STX5, SNX7, MXRA7, PTH2R, NEURL1, ITPR2, OBSCN, SLC3A1, VSIG10, TMEM128, TMPRSS2, SPHK1, SYCN, VTI1B, SLC38A5, SLCO2B1, SLC4A4, SUSD1, REEP4, ACVR1B, DPEP1, CUZD1, GP2, KIRREL2, FA2H, CLEC16A, FCER1G, CLN6, EVI2A, FGFRL1, GPAT2, FKBP11, FES, MAGI1, LEPROTL1, AP3M1, AQP1, AP1S2, ACE2, ANPEP, CFTR, CLDN10, FXYD2, ADCK5, ARL4C, AP4E1, ATP8B1, ATG9A, PIK3AP1, AQP12A, AQP12B, IBTK, LFNG, IL17RA, MRAP, MT-ND4, OR10R2, NDUFB9,
10. Cancer treated: skin cancer
   a. Tissue protected: Colon TSPAN12, AXL, TBC1D9B, TSPAN3, SCN4B, SFT2D2, SNX4, SMAGP, TMEM74B, SLC7A6, TNFSF14, ATP8B2, C2CD2, CORO7, ACY3, GABARAPL2, SLC46A1, SCGN, SLC47A1, PLPP2, SELENBP1, TREM2, SNX24, TMEM263, SMIM24, TMEM94, NAAA, SOCS7, PEX1, PLXNA1, PEMT, SLC25A46, NPDC1, NTRK1, RNF5, NAGPA, NBEA, PIGR, PLEKHA3, TPCN1, TVP23B, TULP3, TFPI, TJP3, WSCD1, RAB27B, RAB20, DLC1, RASD2, PIGU, SERP1, SPCS3, SLC25A36, SLCO4A1, SPPL3, SLC9A3, IFI27, ITLN1, PTGDR, TAOK2, MITD1, MPC2, PEX10, CD46, MGAT1, PCDH11X, KCNK1, MYORG, POMT2, HEG1, ILDR1, LYPLA1, KCNQ1, MGAT4A, MFSD11, CNEP1R1, DENND5B, OBSCN, TMEM159, AATK, MEGF8, PROM1, NUCB2, SERP2, TNFAIP8L3, SELENOT, VPS11, SLC3A1, SIGIRR, VSIG10, VPS28, TVP23C, UTRN, VPS35L, SYTL4, TMEM64, TSPAN31, TMEM41A, UNC13B, ZFYVE9, SYTL2, TMEM53, SLC35A3, MCUR1, OCLN, RNF125, SPHK1, SLC28A3, RAB27A, SYP, SUSD6, PIGZ, PTPRN2, SLC6A12, SLC6A15, SLC19A2, TGFBR1, VTI1B, SIGLEC14, TMEM204, MTG2, NAPG, SUSD1, TECPR1, IMMT, NAPB, RNF186, TMIGD2, VPS41, TMEM39B, ADCK2, ATG2B, CDHR5, ARL4D, ACVR1B, DPEP1, FKRP, FMO5, FKTN, KCNMB3, SLC48A1, FCER1G, GOLPH3, DMXL2, ABCG2, ACSL5, CLCA1, FAM171A1, GLP2R, CEACAM1, GCNT3, B4GALT4, CACNA1B, FPR2, DOK7, FKBP2, CHDH, ADGRF1, HTR1A, CLN6, DGKE, GATM, FZD2, HLA-DQB1, FXYD6, AQP8, CLMN, COQ2, DIAPH1, ELOVL1, WDPCP, CA12, CA4, C6orf89, HHAT, HRH1, GNG11, DGKQ, LRRC37B, CTDNEP1, DGAT1, LIME1, MAGI1, ENTPD8, FPGS, LPP, MALRD1, MAN1C1, AP3M1, CDK14, FER1L6, ANO8, BEST2, BRI3, GAL3ST2, HTR4, B3GNT8, ELOVL7, ERBIN, APOBR, CHPF2, CDHR2, SLC25A13, ACE2, ANPEP, CNTNAP3, CDH17, ATL2, CLCA4, CYB561, ALG2, COG8, ANKRD46, GRAMD1A, ATRN, ASIC5, ADGRL1, B3GNT6, ARL4C, ANO1, ABHD12, AGPS, CDH26, C12orf66, ALG14, CASD1, CHMP2B, B3GAT1, UGT8, FGFR4, IBTK, SMPD4, SLC9A3R2, NCEH1, KCNA5, LFNG, GIPC1, MC4R, GLCE, GFRA2, MCUB, SLC16A9, MSTO1, GNG12, GLRA2, SLC2A6, KIAA2013, KCNK10, NIPAL1, NIPAL4, GPA33, DPY19L1, LZTR1, MUC13, NOD2, HPS6, NEU4, OR10R2, SLC16A4, NDUFB9, ITSN1, PDLIM4, PSKH1, NDST2,
   b. Tissue protected: Lung TGM2, AXL, SLC30A4, TRIM13, SLC22A15, SELENBP1, TREM2, RNF144A, PLXNA1, AGER, PAG1, TVP23B, TFPI, PTPRG, SPCS3, STX5, CD46, KCNQ1, EHD2, SIGIRR, TVP23C, UTRN, RNF125, RNF103, SLC6A15, VTI1B, STEAP1, EHD1, EHD4, FCER1G, CCR7, CYB5A, SLC44A2, ACSL5, AOC3, HLA-DQB1, COQ2, CERS4, AIFM2, AP3M1, AQP4, CYB561, CACNA2D2, ACE, ADGRL1, CACNA1C, CADM1, DAPP1, CAVIN2, IBTK, NCEH1, LAMP3, LFNG, MC4R, EHD3, LDLRAD2, PI4KA, ITSN1, SLC34A2, PSKH1, LRRK2,
   c. Tissue protected: Skin SCN4B, SMAGP, TGFBR3, TMEM263, ENPP1, MIEF2, TULP3, TFPI, REEP2, SPCS3, OBSCN, AATK, TMEM260, ZP2, SLC35A3, RNF125, VTI1B, FA2H, DSP, DMXL2, ANO7, GATM, ATRAID, CD99, ELOVL1, CA12, LPP, AP3M1, B3GNT4, APOBR, ASPRV1, AGPS, OR10R2, PSKH1, SCN4B, MGAT4C, TYR, SLC25A33, NTRK1, RNF139, SUSD4, IFI27, MPC2, KCNQ1, NUCB2, VPS11, VPS28, DCT, MCUR1, PMEL, B3GNT5, DOK7, FZD2, CYB561A3, HCN1, MALRD1, ATRN, BBS5, BBS1, GIPC1, SLC16A9, ALOX12, ,
   d. Tissue protected: Liver TSPAN12, TBC1D9B, VDAC3, TNFSF14, LRP1, TRIM13, SYT13, SELENBP1, RAB17, ENPP1, TMEM94, NAAA, RNF144A, SLC25A40, SLC27A2, SLCO1B3, SLCO1B1, SLC25A33, SLC25A46, SLC25A43, SLC27A5, RNF5, PEX11G, NAGPA, TPCN1, TFPI, RAB20, PIGU, SLC25A36, STX5, SPPL3, KCNQ4, TAOK2, MPC2, CD46, KCNK1, MYORG, HEG1, ILDR1, LYPLA1, KCNQ1, MFSD11, DENND5B, SLC25A15, TMEM159, AATK, MEGF8, UGT2B4, SIGIRR, YIPF1, TMEM260, VAMP1, VPS35L, TMEM64, TNS2, TBCD, UNC13B, TSC2, SLC6A12, SNX19, SERINC1, SLC19A2, VTI1B, TMEM204, SLC35E1, TSPAN2, VPS41, TM4SF4, ACVR1B, FMO5, EVA1A, GHR, FKTN, KCNMB3, SLC48A1, CYP2W1, CYP2D6, CYB5A, ACSL5, FAM171A1, B3GNT5, CACNA1B, CACNA1A, GAS2, SLC2A2, HTR1A, CYP2A7, GATM, EPHX1, FZD2, CYP1A2, FXYD6, COX14, CYP3A4, DIAPH1, CYP2A6, CYP2B6, CERS4, FMO3, HHAT, CYP4V2, KCNJ16, NINJ1, CYP2C8, DHCR24, CDH2, CTDNEP1, FES, KMO, DGAT1, LIME1, MALRD1, CDK14, CYP2C19, BCL2L10, AQP9, ABCA8, BRI3, CYP26B1, ERBIN, CHPF2, CDHR2, SLC25A13, ANPEP, ASGR1, SLC31A1, ABCB11, CYP2A13, COG8, CYP2E1, ASIC5, ANO1, BBS1, ASPHD1, EPB41L5, ASGR2, CYP3A43, CHMP2B, DAPP1, INTS2, FGFR4, IBTK, LHFPL2, MFAP3L, GIPC1, GLCE, MSTO1, GK, SLC2A6, NIPAL4, IGFLR1, DPY19L1, HPS6, ABCC2, DPP4, PSKH1, SLC10A1, NPC1L1,
   e. Tissue protected: Kidney (glomeruli) CORO7, SYT13, TGFBR3, TREM2, RAB17, SLC45A1, RNF144A, PODXL, KIRREL1, PDGFRB, TSPAN33, TFPI, KSR2, RNF139, PTPRG, MRGPRF, TPSG1, STX5, PLA2R1, CD46, MGAT1, PTH2R, POMT2, MAG12, MAP6, EHD2, LDLRAD3, NPHS2, UTRN, TNS2, SNCA, NAPG, PTPRO, SLC35B1, VASN, ARL4D, EHD1, HYAL2, EHD4, ADGRF1, CD34, EVI2A, HLA-DQB1, ENG, CD99, CR1, LPP, CAPN2, ANO8, CRB3, ATL2, CLCA4, EPB41L5, AKAP12, CACNA1C, ABCC1, SLC9A3R2, LIMS2, ITGA8, EHD3, NIPAL4, LDLRAD2, LZTR1, NPHS1, HPS6, PI4KA, NEU4, DYSF, DPP4, PSKH1,
   f. Tissue protected: Kidney (tubules) TSPAN12, VNN1, AXL, TBC1D9B, VDAC3, SCN4B, SFT2D2, SNX4, TRPM3, TMEM74B, TREH, TNFSF14, C2CD2, ACY3, FCAMR, GABARAPL2, TM4SF18, UBE2J1, SLC47A1, SELPLG, PLPP2, SELENBP1, TREM2, RAB17, TMEM263, ENPP1, SMIM24, SLC5A12, SLC5A2, TMEM94, SLC12A1, NAAA, RNF144A, SLC25A40, SLC27A2, SMIM1, PEX1, PEMT, SLC6A19, SLC22A11, SIPR2, SLC34A3, SLC25A33, SLC25A46, ARHGAP5, RNF5, MIEF2, PEX11G, SRD5A3, NAGPA, NEGR1, PTH1R, PDGFC, NBEA, PIGR, RGS16, PLLP, TPCN1, TFPI, WSCD1, VHL, PTPRD, RAB27B, RAB20, DLC1, RASD2, MPP6, PIGU, LIN7A, MRGPRF, UCHL1, SERP1, SPCS3, MGAT4B, SLC34A1, UPK3BL2, RASA4, S LC25A36, STX5, PRICKLE1, SLCO4A1, SPPL3, KCNQ4, MITD1, MPC2, CD46, KCNK1, NAT8, MYORG, POMT2, ILDR1, LYPLA1, KCNQ1, MGAT4A, MFSD11, CNEP1R1, DENND5B, MPP5, SLC25A15, OBSCN, TMEM159, LDLRAD3, AATK, MEGF8, PROM1, UGT2B4, ATP6V1B1, SLC22A6, SERP2, TMEM72, TNFAIP8L3, VPS11, SLC3A1, SIGIRR, VSIG10, VPS28, ATP6V0A4, SLC7A7, TMEM80, TMEM260, VAMP1, UTRN, VPS35L, ZP2, VAC14, QTRT1, TMEM252, TMEM174, TMEM64, TTC7B, TSPAN31, UNC13B, ZFYVE9, SYTL2, TMPRSS2, TMEM255B, UPK3BL1, TSC2, SLC28A1, MCUR1, SLC22A2, OCLN, SPHK1, SLC28A3, SCAMP5, SLC22A8, SLC13A3, PIGZ, SLC6A12, SNX19, RUFY3, RAB11FIP5, SLC19A2, SLC6A18, SLC13A2, TGFBR1, TMEM213, VTI1B, SYT7, TMEM204, TMEM184A, SLC4A4, SLC25A23, NAPG, SRPRB, TECPR1, PKHD1, PRRT3, IMMT, SLC35E1, SLC35G2, OPRD1, NAPB, TRPV5, TSPAN2, TMIGD2, VASN, VPS41, TMEM39B, ADCK2, CDHR5, ARL4D, ENPP6, GGT1, DPEP1, ENPP3, CUBN, FKRP, EVA1A, GHR, GREB1, HYAL2, FKTN, GSDMC, KCNMB3, SLC48A1, CPNE6, CYB5A, SLC44A2, DMXL2, GJA5, ACSL5, FAM171A1, B4GALT4, B3GNT5, CACNA1B, GAS2, DOK7, FKBP2, SLC2A2, CHDH, HTR1A, CALCR, CLN6, CREB3L2, EMC10, GATM, FZD2, ATRAID, CASR, FOLH1, FXYD6, CLMN, CNTFR, COQ2, COX14, CDIPT, ELOVL1, CA12, CERS4, HHAT, HRH1, GNG11, DGKQ, KCNJ16, CDH2, CTDNEP1, FES, KMO, SLC2A5, LRFN4, LIME1, MAGI1, MALRD1, AP3M1, CDK14, BSND, BCL2L10, CAPN2, ANO8, AQP6, CDC42EP5, MPP1, CHPF2, CDHR2, SLC25A13, ANK2, ANPEP, SLC7A9, CPM, CLDN10, SLC31A1, ATL2, CLCA4, ALG2, S LC25A31, FXYD2, ACE, CMKLR1, ACKR3, CLDN2, COG8, COX7B, ANKRD46, GRAMD1A, ATRN, ADGRL1, TMEM63B, ACVRL1, BBS5, ARL4C, ANXA13, BBS1, ASPHD1, EPB41L3, AGPS, C12orf66, ATP12A, ATG9A, CACNA1C, CYP3A43, CDH16, CHMP2B, CADM1, B3GAT1, UGT8, INTS2, FGFR4, ABCC5, SMPD4, MOXD1, PDZD3, HAS3, KCNA5, LFNG, LHFPL2, GIPC1, NDUFS1, MC4R, GLCE, GFRA2, KCND1, KCNK5, MCUB, SLC16A9, MSTO1, GNG12, GK, SLC2A6, KIAA2013, NIPAL1, FMO1, DPY19L1, LRP2, LZTR1, ALOX12, HPS6, PI4KA, OR10R2, SLC16A4, GRIN2D, NDUFB9, ITSN1, ABCC2, MDGA2, PDLIM4, DYSF, DPP4, PSKH1, LRRK2, NDST2,
   g. Tissue protected: Heart (cardiomyocytes) TGM2, TBC1D9B, TCTN2, TSPAN3, VDAC3, SLC30A4, SLC27A6, SCN4B, SMAGP, SLC7A6, UCP3, CAP2, GABARAPL2, MGAT4C, SLC14A1, TMEM106B, SLC22A15, RAB17, SEZ6L2, SNX24, SUSD3, TMEM94, PITPNM1, RNF144A, SLC4A10, PLXNA1, SGCB, PEMT, SLC25A33, MIEF2, SRD5A3, NAGPA, NEGR1, BVES, P2RX6, TPCN1, TSPAN33, TFPI, RAB20, POPDC2, P2RY6, SERP1, SGCG, SLCO4A1, SRL, KCNQ4, MITD1, MPC2, MGAT1, MYORG, KCNQ1, MFSD11, CNEP1R1, DENND5B, DMPK, SLC25A15, OBSCN, TMEM159, AATK, MEGF8, NUCB2, PRIMA1, SERP2, SIPA1, RYR2, VPS11, YRDC, TMEM143, UTRN, ZP2, VAC14, TMEM64, TSPAN31, PTPRB, TSC2, RAB11FIP4, SUSD6, SLC6A12, SNX19, SERINC1, SLC19A2, VTI1B, SLCO2B1, TMEM204, PPM1L, SRPRB, TECPR1, TEX261, IMMT, VASN, VPS41, TMEM39B, ATG2B, GREB1, CKMT2, CYP2W1, DSP, DMXL2, FAM171A1, CACNA2D1, CDH15, DOK7, FNDC5, GNB5, HTR1A, CAVIN4, EMC10, SLC2A4, DGKE, HHATL, FZD2, FXYD6, CHRNA1, CNTFR, CD248, CDIPT, ELOVL1, WDPCP, GPAT2, C6orf89, HHAT, CYP4V2, DGKQ, CAMK2B, KCNJ16, LRRC37B, CDH2, CTDNEP1, FES, LIME1, LEPROTL1, AP3M1, ABHD16A, DMD, ERBIN, CDC42SE1, CHPF2, CDHR2, SLC25A13, ADAM28, ANK2, CNTNAP3, SLC31A1, CSF3R, ATL2, CAV3, COG8, CPT1B, COX7B, ANKRD46, AKAP6, ADGRG6, ADRB1, ARL4C, BBS1, AP4E1, BCL2L13, ATG9A, CACNA1C, UGT8, ABCC5, SMPD4, NCEH1, KCNA5, MYZAP, LFNG, MFAP3L, NCAM1, GIPC1, G PR182, NDUFS1, MC4R, GLCE, KCND1, MSTO1, GPR15, MLIP, NIPAL4, S LC16A10, IGFLR1, LDLRAD2, DPY19L1, PI4KA, ICA1, OR10R2, SLC16A4, NDUFB9, ITSN1, PRKCA, DES, DYSF, GPR63, PSKH1,
   h. Tissue protected: Thyroid TSPAN12, TBC1D9B, TSPAN3, SLC30A4, SCN4B, SFT2D2, SMAGP, TMEM74B, TNFSF14, C2CD2, TMEM100, TMEM106B, VPS33B, SLC22A15, TGFBR3, SELENBP1, TREM2, RAB17, TMEM263, ENPP1, TMEM94, RNF144A, SCNN1D, RHOA, PLXNA1, PEMT, SLC25A33, SLC25A46, NPDC1, NTRK1, RNF5, MIEF2, NAGPA, NBEA, TPO, TPCN1, TVP23B, TSPAN33, TULP3, TFPI, TJP3, LMAN2, PIGU, RNF139, SERP1, SPCS3, SUSD4, IFI27, MPC2, CD46, KCNK1, PSD3, POMT2, HEG1, ILDR1, KCNQ1, MGAT4A, MFSD11, CNEP1R1, DENND5B, OBSCN, AATK, PROM1, NUCB2, PRIMA1, SERP2, VPS28, TMEM260, TVP23C, UTRN, VPS35L, SYTL4, TBCD, TMEM41A, UNC13B, ZFYVE9, TMEM53, TSC2, OCLN, RNF125, SLC26A4, SLC19A2, VTI1B, RNFT2, PPM1L, SLC25A23, NAPG, SUSD1, TECPR1, IMMT, SLC35E1, VPS41, TMEM39B, IYD, DUOX1, GHR, FKTN, KIFC3, FCER1G, CYP2W1, F AM171A1, B3GNT5, FKBP2, CLN6, DGKE, HLA-DQB1, FXYD6, CD99, COQ2, DIAPH1, C6orf89, HCN1, IPCEF1, KCNJ16, CTDNEP1, FES, DGAT1, LIME1, MAGI1, AP3M1, CDK14, FZD5, ENTPD1, FIBP, ERBIN, CHPF2, AAK1, CLCA4, ALG2, ACKR3, COG8, ARRDC1, ATRN, ADGRL1, ACVRL1, BBS5, BBS1, AP4E1, AGPS, CDH16, CADM1, CAVIN2, IBTK, NLGN1, MTNR1B, LFNG, LHFPL2, NCAM1, GIPC1, MC4R, GFRA2, MSTO1, GNG12, GK, IGFLR1, FAM234B, HPS6, ICA1, OR10R2, GRIN2D, PDLIM4, PSKH1,
   i. Tissue protected: Pancreatic endocrine cells (Islets) TSPAN12, TSPAN3, VDAC3, SMAGP, STXBP1, SCGN, TGFBR3, PLPP2, STX1A, SEZ6L2, NAAA, RNF144A, RHOA, RTN1, NPDC1, NTRK1, SCG3, MIEF2, NBEA, TVP23B, TFPI, VHL, MPP6, PTGDR2, UCHL1, SERP1, KCNQ4, TAOK2, MPC2, PTPRN, CD46, PTH2R, KCNK1, POMT2, MGAT4A, MFSD11, OBSCN, AATK, NECAB2, NUCB2, SLC17A6, ATP6V1B1, SHISAL2B, VPS11, SV2A, YRDC, TVP23C, VAMP1, VAC14, TSPAN7, TMEM64, ZFYVE9, SLC30A8, TMEM255B, TMEM53, RNF103, SYP, PTPRN2, RUFY3, SLC19A2, VTI1B, SIGLEC14, PPM1L, NAPG, TECPR1, TEX261, IMMT, SLC35G2, NAPB, VPS41, ATG2B, GREB1, DGCR2, CYP2W1, DMXL2, FAM171A1, B3GNT5, DOK7, GNAO1, FAM169A, CASR, CD99, CNTFR, COQ2, DIAPH1, FKBP11, C6orf89, CDH2, CTDNEP1, MALRD1, FIBP, ENO2, ATP9A, CLU, CYB561, ALG2, FXYD2, ATP1A2, CADPS, COG8, ARRDC1, ASIC5, ADGRL1, BBS1, EPB41L3, CDH26, CADM1, GAD2, DAPP1, ALOX5, NLGN1, LFNG, GIPC1, MC4R, GLCE, KCND1, MSTO1, IGFLR1, LDLRAD2, HPS6, PI4KA, OR10R2, LRRC24, MDGA2, PDLIM4, DPP4, PSKH1,
   j. Tissue protected: Pancreatic exocrine cells TBC1D9B, SMAGP, TNFSF14, C2CD2, CHRM3, GABARAPL2, UBE2J1, SELPLG, PLPP2, RAB17, TMEM263, ENPP1, STIMATE, NAAA, SLC4A10, PLXNA1, PEMT, TMEM97, NTRK1, RNF5, NBEA, PLEKHA3, TVP23B, TSPAN33, TFPI, TJP3, VHL, RAB27B, MPP6, RNF139, SPCS3, UPK3BL2, SLC25A36, STX5, IFI27, MITD1, PTH2R, NEURL1, KCNK1, HEG1, KCNQ1, MGAT4A, OBSCN, TMEM159, AATK, MEGF8, PROM1, NUCB2, PRIMA1, SELENOT, VPS11, SLC3A1, SIGIRR, VSIG10, YRDC, TMEM80, TVP23C, VAMP1, UTRN, VAC14, SYTL4, TSPAN31, PTPRB, SYTL2, TMPRSS2, TMEM255B, TMEM53, OCLN, SPHK1, SYCN, SLC28A3, SUSD6, SERINC1, SLC19A2, VTI1B, SLCO2B1, TMEM204, MTG2, SLC4A4, NAPG, SRPRB, SUSD1, PKHD1, IMMT, ARL4D, ACVR1B, DPEP1, CUZD1, FKTN, GP2, KIFC3, KIRREL2, FA2H, FCER1G, GOLPH3, DMXL2, FAM171A1, B3GNT5, CACNA1B, DOK7, FKBP2, CLN6, EVI2A, GATM, EPHX1, AQP8, CLMN, CNTFR, COQ2, DIAPH1, WDPCP, GPAT2, FKBP11, CA12, CERS4, CYP4V2, DGKQ, CTDNEP1, FES, DGAT1, MAGI1, LEPROTL1, MALRD1, AP3M1, CYP26B1, KDELR3, ELOVL7, ERBIN, CHPF2, SLC25A13, CRB3, ACE2, ANPEP, CFTR, CLDN10, CYB561, ALG2, FXYD2, ACKR3, CCR4, COG8, ANKRD46, ARRDC1, ADGRL1, BBS5, ARL4C, AP4E1, ASPHD1, ATG9A, CADM1, DAPP1, ARFGAP3, AQP12A, AQP12B, FGFR4, IBTK, SMPD4, MOXD1, KCNA5, LFNG, GIPC1, MC4R, GLCE, MCUB, SLC2A6, SLC16A10, FAM234B, NOD2, HPS6, MYOC, OR10R2, SLC16A7, NDUFB9, PDLIM4, PSKH1,
11. Cancer treated: endometrial cancer
   a. Tissue protected: Colon TMED6, UGT2A3, TMEM138, SLC7A6, TMEM60, SCGN, SMIM24, SOCS7, RAC1, PCDHGA10, SLC26A1, LDLR, PILRA, RAC3, DLC1, PLCB3, SDR16C5, RDH13, RAB28, SEL1L3, TMEM45A, SERP1, SPPL3, MAPKAP1, SLC9A3, PTGDR, PRKN, MYORG, PLPPR2, PLPP7, OBSCN, SERP2, SLC3A1, UGT2B17, VSIG10, TMEM106C, VAMP5, UTRN, VPS35L, UGT2B28, TMEM253, TOMM40L, UNC13B, SYTL2, TMX2, UGT2B15, RAB43, SYP, PIGZ, PTPRN2, SLC39A2, SLC39A14, SLC6A15, SNX6, TGFBR1, SLC38A5, REEP4, MSMO1, NAPB, TSNARE1, TRAF3IP3, TYRO3, TMEM39B, CDHR5, EFHD1, DPEP1, EMC9, GSDMB, CX3CR1, GRAP, PHKA1, ABCG1, ABCG2, CD177, CLCA1, GLP2R, FPR2, CLN6, DMTN, AQP8, CA4, DGKQ, IGSF11, LIME1, LPP, MAN1C1, EPHA10, FER1L6, ANO8, BEST2, BRI3, GAL3ST2, B3GNT8, FAM162A, DENND5A, ACE2, CNTNAP3, CDH17, AFG1L, B3GNT6, TMEM30B, DCXR, ABHD12, C12orf66, COMTD1, HSD3B7, ALG14, B3GAT1, PYCARD, SLC16A6, KCNN3, GFRA2, MCUB, KIAA2013, GPA33, DPY19L1, MUC13, NOD2, OR10R2, MAPK8IP1,
   b. Tissue protected: Lung SLC30A4, TRIM13, VIPR2, SLC22A15, SLC25A17, AGER, SDR16C5, RECK, PTPRG, EHD2, VAMP5, UTRN, PRRG2, PSD4, RAB43, SLC6A15, STEAP1, PHLPP2, EHD1, EHD4, CCR7, CYB5A, CAVIN1, GPC5, AOC3, AIFM2, CLIC2, FCAR, ELMO2, CAV1, CACNA2D2, ACE, AP3S1, CAV2, AP3S2, LAMP3, EHD3, NSMF, LRRK2,
   c. Tissue protected: Skin TMEM138, CLEC10A, PERP, VIPR2, RXFP1, TGFBR3, ENPP1, RAC1, PPP1R16B, RAC3, TMEM40, MTMR2, SDR16C5, RAB28, SLC10A6, LYPD3, KRT1, SLC8A1, OBSCN, TRPM2, ZP2, TMX2, LY6D, MSMO1, TSNARE1, TRAF3IP3, EMC9, GJB4, IGSF8, FA2H, PHKA1, DSP, CAVIN1, CD177, ANO7, ATRAID, DMTN, CAPNS2, DSC3, LPP, EPHA10, B3GNT4, DSG1, CAV1, CAV2, ASPRV1, DSG3, DCXR, CNPPD1, MAP1LC3B, OR10R2, MGAT4C, TYR, SLC26A1, SEL1L3, TMEM45A, ICAM3, PLPP7, DCT, RHCG, S100A8, PMEL, REEP4, TYRO3, GRAP, GPC5, CYB561A3, FAT2, FAM210A, HCN1, MALL, CLEC2B, FAM162A, BST1, AFG1L, CLEC2A, COMTD1, HSD3B7, PYCARD, ALOX12, MAPK8IP1, ,
   d. Tissue protected: Liver TMEM138, CLEC10A, TRIM13, STBD1, ENPP1, SLC25A40, SLCO1B3, SLCO1B1, SLC27A5, PPP1R16B, LDLR, RDH13, RDH16, TMEM45A, SPPL3, MAPKAP1, MYORG, PLPPR2, SLC8A1, MME, UGT2B4, TFR2, VAMP5, VPS35L, TNS2, TBCD, UNC13B, RAB43, SLC39A2, SLC39A14, MSMO1, TSNARE1, EVA1A, CYP2W1, CYP2D6, CYB5A, ABCG1, CD177, CACNA1A, FYN, GAS2, SLC2A2, CYP2A7, CYP1A2, CYP3A4, FAM210A, CYP2A6, CYP2B6, FMO3, CYP4V2, ABCB4, NINJ1, CYP2C8, DHCR24, CLEC2B, FES, LIME1, CYP2C19, BCL2L10, AQP9, ABCA8, BRI3, ENPEP, ASGR1, ABCB11, CYP2A13, TMEM30B, DCXR, CMTM8, ASGR2, CYP3A43, HSD3B7, CYP2C9, SLC16A6, MFAP3L, DPY19L1, ABCC2, SLC10A1, NPC1L1,
   e. Tissue protected: Kidney (glomeruli) TMEM138, ZAP70, VIPR2, KDR, TGFBR3, SLC45A1, KSR2, RECK, PTPRG, MRGPRF, TPSG1, PLA2R1, PRKN, PTH2R, PNPLA3, EHD2, MME, NPHS2, VAMP5, UTRN, TNS2, PRRG2, SNCA, PSD4, PTPRO, SREBF1, TYRO3, EHD1, HYAL2, CX3CR1, EHD4, CAVIN1, CD34, DMTN, ENG, FAM210A, CLEC2B, LPP, CAPN2, ANO8, AQP1, CAV1, ENPEP, CAV2, LIMS2, GPR153, ITGA8, EHD3, NPHS1, KIAA0040,
   f. Tissue protected: Kidney (tubules) VNN1, TRPM3, TREH, UBE2J1, SELPLG, ENPP1, SMIM24, SLC5A12, SLC5A2, SLC12A1, SLC25A40, SLC6A19, SLC22A11, SLC34A3, SLC26A1, NEGR1, PPP1R16B, PTH1R, LDLR, RHBG, PARVB, DLC1, RDH13, RAB28, KCNH8, LIN7A, MRGPRF, SEL1L3, SERP1, MGAT4B, SLC34A1, UPK3BL2, SPPL3, MAPKAP1, PRKN, NAT8, MYORG, PLPPR2, PNPLA3, SLC8A1, MME, OBSCN, UGT2B4, SLC22A6, SERP2, TMEM72, UGT3A1, XPNPEP2, SLC3A1, VSIG10, ATP6V0A4, SLC7A7, TMEM106C, UTRN, VPS35L, ZP2, QTRT1, TMEM252, TMEM174, UNC13B, RHCG, SYTL2, TMPRSS2, TMX2, UPK3BL1, SLC28A1, SLC22A2, SCAMP5, SLC22A8, SLC13A3, PIGZ, SLC39A2, SLC39A14, SNX6, SLC6A18, SLC13A2, TGFBR1, TMEM213, SLC4A4, SREBF1, REEP4, SLC35G2, OPRD1, NAPB, TSNARE1, TYRO3, TSPAN16, TMEM39B, CDHR5, ENPP6, EFHD1, DPEP1, EMC9, CUBN, EVA1A, HYAL2, GSDMC, CX3CR1, GRAP, PHKA1, CPNE6, CYB5A, ABCG1, GJA5, GPC5, DAB2, FAM151A, GAS2, SLC2A2, CLN6, CREB3L2, TM7SF2, ATRAID, CASR, DMTN, FAM210A, DGKQ, IGSF11, CLEC2B, FES, LIME1, SLC7A8, BSND, BCL2L10, CAPN2, ANO8, AQP6, AQP1, CDC42EP5, MPP1, DENND5A, SH3BP5, AFG1L, ENPEP, SLC7A9, SLC25A31, FXYD2, ACE, CLDN2, TMEM63B, DCXR, AP1S3, EPB41L3, C12orf66, COMTD1, ATP12A, CYP3A43, HSD3B7, B3GAT1, HAS3, SLC43A2, SLC16A6, GFRA2, MCUB, KIAA2013, MAP1LC3B, FMO1, DPY19L1, LRP2, ALOX12, CHL1, NXPE2, OR10R2, GRIN2D, NSMF, MAPK8IP1, ABCC2, LRRK2,
   g. Tissue protected: Heart (cardiomyocytes) SLC30A4, TMEM138, SLC7A6, UCP3, CD36, MGAT4C, RASL10B, SLC14A1, TMEM106B, VIPR2, SLC22A15, SLC25A17, STBD1, SUSD3, SLC4A10, NTRK3, NEGR1, PPP1R16B, BVES, PARVB, POPDC2, PLN, RAB28, SERP1, SGCG, SRL, MAPKAP1, LANCL2, MYORG, PLPP7, SLC8A1, DMPK, OBSCN, SERP2, SIPA1, YRDC, TMEM106C, TMEM143, VAMP5, UTRN, ZP2, TOMM40L, PRRG2, TMX2, SLC39A14, SLCO2B1, ST8SIA2, SCN7A, MSMO1, TSNARE1, TMEM39B, EMC9, GRAP, CKMT2, PHKA1, CYP2W1, DSP, CDH15, RASGRP2, CAVIN4, HHATL, FAM168B, CHRNA1, GPAT2, CYP4V2, DGKQ, CAMK2B, IGSF11, CLEC2B, FES, LIME1, LEPROTL1, ABHD16A, EPHA10, CDC42SE1, ATP1A3, DENND5A, CNTNAP3, CAV1, AFG1L, CAV2, CAV3, CDH13, AKAP6, ADGRG6, ADRB1, AP1S3, BCL2L13, CNPPD1, COMTD1, SLC16A6, MFAP3L, NCAM1, GPR182, GPR15, MLIP, DPY19L1, OR10R2, DES,
   h. Tissue protected: Thyroid SLC30A4, TMEM106B, VIPR2, SLC22A15, TGFBR3, ENPP1, SCNN1D, NTRK3, NUS1, TPO, RHBDD2, RDH13, RAB28, SEL1L3, SERP1, MAPKAP1, PRKN, LANCL2, PSD3, SLC8A1, OBSCN, SERP2, TMEM106C, UTRN, VPS35L, TBCD, UNC13B, PRRG2, RAB43, SLC39A2, RNFT2, SREBF1, TSNARE1, TYRO3, TMEM39B, IYD, DUOX1, EMC9, GRAP, CYP2W1, ABCG1, CAVIN1, DAB2, FYN, CLN6, HCN1, MALL, GDAP1, FES, LIME1, CLIC2, AAK1, CAV1, CAV2, CD300A, DCXR, AP1S3, CMTM8, COMTD1, NCAM1, GFRA2, MAP1LC3B, OR10R2, GRIN2D, MAPK8IP1,
   i. Tissue protected: Pancreatic endocrine cells (Islets) SCGN, SLC25A17, TGFBR3, STX1A, RTN1, SCG3, PTGDR2, SERP1, PTPRN, PTH2R, SLC8A1, OBSCN, NECAB2, SLC17A6, YRDC, TSPAN7, SLC30A8, PRRG2, SYP, PTPRN2, SLC39A2, SLC35G2, MSMO1, NAPB, DGCR2, PHKA1, CYP2W1, GNAO1, CASR, CD82, SLC7A8, DGKZ, ENO2, ELMO2, FXYD2, ATP1A2, CADPS, CFC1, EPB41L3, GAD2, ALOX5, MAP1LC3B, OR10R2,
   j. Tissue protected: Pancreatic exocrine cells TMED6, RASL10B, UBE2J1, SELPLG, ENPP1, STIMATE, SLC4A10, PPP1R16B, SEL1L3, UPK3BL2, PTH2R, NEURL1, OBSCN, SLC3A1, VSIG10, YRDC, TMEM106C, VAMP5, UTRN, TMEM128, SYTL2, TMPRSS2, TMX2, SYCN, SLC39A2, SLC39A14, SLC38A5, SLCO2B1, SLC4A4, SCN7A, REEP4, DPEP1, EMC9, CUZD1, KIRREL2, FA2H, GRAP, CLN6, AQP8, FAM210A, GPAT2, CYP4V2, DGKQ, FES, LEPROTL1, AQP1, KDELR3, FAM162A, ACE2, FXYD2, CAV2, CD300A, CMTM8, COMTD1, PIK3AP1, AQP12A, AQP12B, MCUB, NOD2, OR10R2,
12. Cancer treated: breast cancer
   a. Tissue protected: Colon ZDHHC2, UGT2A3, TMEM138, TMEM60, C2CD2, RMDN2, LPCAT2, SLC26A2, SOCS7, NDUFS8, SLC19A3, PLPP1, WHAMM, DLC1, PLCB3, SLC25A20, PVR, RAB3B, SEL1L3, TMEM45A, SPPL3, STYK1, MAPKAP1, SLC9A3, IFI27, ITLN1, PTGDR, PEX10, OSBPL7, LRRC55, PLPP7, OBSCN, PLOD1, UGT2B17, VSIG10, UGT2B28, TMEM69, SYTL3, TMEM253, UGT2B15, SLC22A1, SYP, PIGZ, SLC39A2, S LC39A14, SLC6A15, SNX6, SDCBP2, REEP4, TMEM171, ATG2B, CRK, CDHR5, DPEP1, GSDMB, ABCG1, ABCG2, ACSLS, CD177, CLCA1, GLP2R, CEACAM1, GBA2, FPR2, HTR1A, GPR34, DMTN, CEACAM5, CA4, GNG11, IGSF11, EPHA10, FER1L6, BEST2, BRI3, GAL3ST2, B3GNT8, CDHR2, ACE2, CNTNAP3, CDH17, C1orf210, ASIC5, DCXR, EPB41L2, ABHD12, CDH26, IBTK, SLC16A9, KIAA2013, ITGB4, GPA33, LZTR1, MUC13, LBR, OR10R2, NDUFB9,
   b. Tissue protected: Lung SLC30A4, VIPR2, SLC22A15, SLC25A17, LPCAT2, AGER, WHAMM, PTPRG, SGIP1, EHD2, SLC6A15, STEAP1, PHLPP2, EHD1, EHD4, CCR7, CAVIN1, ACSL5, AIFM2, CLIC2, AIG1, FCAR, ELMO2, CAV1, AQP4, CACNA2D2, ACE, CAV2, ANXA1, EPB41L2, CAVIN2, IBTK, LAMP3, EHD3, MMP2, NSMF,
   c. Tissue protected: Skin TMEM243, ZDHHC2, TMEM138, CLEC10A, ACER3, PERP, VIPR2, PCDHB5, TMEM40, REEP2, TMEM134, SLC10A6, KRT1, PLA2G4E, OBSCN, RMC1, SRC, ZP2, TMEM69, SYTL3, TACSTD2, LY6D, TMEM171, CRK, FGFR2, IGSF8, FA2H, DSP, CAVIN1, CD177, GPR34, DMTN, CD99, DSC3, FGFBP1, FADS3, EPHA10, B3GNT4, DSG1, CAV1, CAV2, ANXA1, ASPRV1, ORAI1, DSG3, DCXR, TACR1, OR10R2, TMEM243, MS4A8, SEL1L3, TMEM45A, IFI27, LRRC55, PLPP7, DCT, RHCG, SDCBP2, RYK, PMEL, REEP4, CYB561A3, FAT2, HCN1, EPB41L2, SLC16A9, ITGB4, ,
   d. Tissue protected: Liver TMEM138, LRP1, CLEC10A, RMDN2, SYT13, SLC25A40, SLCO1B3, SLCO1B1, SLC25A43, SLC27A5, SLC19A3, RETSAT, SLC25A20, TMEM45A, SPPL3, MAPKAP1, LRRC55, MME, UGT2B4, TFR2, UGT1A6, SLC22A1, SLC39A2, SLC39A14, TMEM171, CRK, FGFR2, EVA1A, CYP2D6, ABCG1, ACSL5, CD177, GBA2, CACNA1A, GAS2, SLC2A2, HTR1A, CYP2A7, GPR34, CYP1A2, CYP3A4, CYP2B6, FM03, NINJ1, CYP2C8, CDH2, FES, CYP2C19, BCL2L10, AQP9, ABCA8, AIG1, BRI3, CDHR2, ENPEP, ASGR1, ABCB11, CTSL, ASIC5, ACAD11, DCXR, HSD11B1, ASGR2, CYP2C9, IBTK, GRB14, ITGB4, ABCC2, DPP4, NPC1L1,
   e. Tissue protected: Kidney (glomeruli) TMEM138, ZDHHC6, ACER3, VIPR2, KDR, SYT13, SLC45A1, PCDHB5, PODXL, KIRREL1, PLPP1, TRPV6, WHAMM, ITGB1, KSR2, PVR, PTPRG, TPSG1, SCUBE1, PLA2R1, PTH2R, MAP6, EHD2, MME, NPHS2, RMC1, SLC22A1, SNCA, SDCBP2, RYK, PTPRO, SLC35B1, TMEM171, VASN, EHD1, HYAL2, EHD4, CAVIN1, CD34, GPR34, DMTN, ENG, CD99, GPRC5B, FADS3, AQP1, CAV1, ENPEP, CAV2, ANXA1, EPB41L2, ABCC1, LIMS2, GPR153, ITGA8, EHD3, LZTR1, NFXL1, DYSF, DPP4,
   f. Tissue protected: Kidney (tubules) TMEM243, TTYH3, ZDHHC2, TRPM3, ZDHHC6, TREH, C2CD2, ACER3, FCAMR, RMDN2, SLC5A12, SLC12A1, SLC25A40, PCDHB5, SLC6A19, S1PR2, NDUFS8, SLC34A3, SLC19A3, MGAM, ARHGAP5, RETSAT, NEGR1, RHBG, VPS26B, VHL, WHAMM, PTPRD, DLC1, SLC25A20, KCNH8, KCNJ1, LIN7A, SEL1L3, UCHL1, MGAT4B, UPK3BL2, TMEM132E, PRICKLE1, SPPL3, MAPKAP1, OSBPL7, LRRC55, NAT8, KIRREL3, MME, OBSCN, UGT2B4, SLC22A6, TMEM72, UGT3A1, VSIG10, ATP6V0A4, SLC7A7, SRC, ZC3H12A, ZP2, QTRT1, UGT1A6, TMEM252, TMEM174, TTC7B, RHCG, TMPRSS2, UPK3BL1, SLC28A1, SLC22A1, SLC22A2, SCAMP5, SLC22A8, SLC13A3, PIGZ, SLC39A2, SLC39A14, SNX6, SLC6A18, SLC13A2, TMEM213, SLC4A4, SDCBP2, RYK, REEP4, SLC35G2, TMEM171, VASN, CRK, CDHR5, ENPP6, DPEP1, ENPP3, CUBN, EVA1A, HYAL2, GSDMC, ABCG1, GJA5, ACSL5, CDH6, FAM151A, GBA2, GAS2, SLC2A2, HTR1A, CREB3L2, GPR34, CASR, FOLH1, DMTN, CLIC4, GNG11, GPRC5B, IGSF11, CDH2, FES, SLC7A8, BSND, BCL2L10, AQP6, AQP1, CDC42EP5, MPP1, CDHR2, SH3BP5, ENPEP, FXYD2, ACE, CLDN2, COX7B, C1orf210, DCXR, ATP12A, CDH16, ABCC5, SLC43A2, KCNK5, SLC16A9, KIAA2013, LZTR1, NXPE2, OR10R2, NDUFB9, NSMF, ABCC2, DYSF, DPP4,
   g. Tissue protected: Heart (cardiomyocytes) TTYH3, SLC30A4, TMEM138, UCP3, VIPR2, RMDN2, SLC22A15, SLC25A17, PITPNM1, PCDHB5, SGCB, NDUFS8, SLC19A3, RETSAT, NEGR1, P2RY6, S LC25A20, PLN, PVR, SGCG, SRL, MAPKAP1, LRRC55, PLPP7, OBSCN, SIPA1, TMEM143, ZC3H12A, ZP2, SLC22A1, RAB11FIP4, SLC39A14, SLCO2B1, TEX261, TMEM171, VASN, ATG2B, FGFR2, CKMT2, DSP, CDH6, CACNA2D1, CDH15, GBA2, FNDC5, RASGRP2, HTR1A, HHATL, FAM168B, GPR34, CHRNA1, CD248, GPAT2, CAMK2B, IGSF11, CDH2, FES, LEPROTL1, EPHA10, CDC42SE1, CDHR2, ADAM28, CNTNAP3, CAV1, CSF3R, CAV2, CAV3, CPT1B, COX7B, CDH13, ORAI1, ADGRG6, ADRB1, ACAD11, BCL2L13, ABCC5, GRB14, GPR182, GPR15, MLIP, ITGB4, NFXL1, OR10R2, NDUFB9, DES, DYSF,
   h. Tissue protected: Thyroid SLC30A4, ZDHHC6, C2CD2, TMEM100, VIPR2, RMDN2, SLC22A15, SLC5A5, LPCAT2, PCDHB5, SLC19A3, RETSAT, NUS1, TPO, TRPV6, VPS26B, PVR, SEL1L3, MAPKAP1, IFI27, OSBPL7, PSD3, LRRC55, OBSCN, ZC3H12A, SYTL3, SLC26A4, SLC39A2, SDCBP2, RYK, IYD, FGFR2, KIFC3, ABCG1, CAVIN1, CDH6, GBA2, GPR34, CD99, HCN1, IPCEF1, GDAP1, FES, CLIC2, AAK1, CAV1, CAV2, DCXR, CDH16, CAVIN2, IBTK, MTNR1B, OR10R2, IKBIP,
   i. Tissue protected: Pancreatic endocrine cells (Islets) ZDHHC2, STXBP1, SLC25A17, STX1A, RTN1, SCG3, VHL, PTGDR2, PVR, RAB3B, UCHL1, PTPRN, PTH2R, LRRC55, OBSCN, NECAB2, SLC17A6, SHISAL2B, SV2A, TMEM69, TSPAN7, SLC30A8, SYP, SLC39A2, SDCBP2, TEX261, SLC35G2, ATG2B, FGFR2, DGCR2, CDH6, GBA2, GNAO1, FAM169A, GPR34, CASR, CD99, CDH2, SLC7A8, ELMO2, FXYD2, CADPS, ASIC5, CFC1, CDH26, GAD2, ALOX5, OR10R2, DPP4,
   j. Tissue protected: Pancreatic exocrine cells ZDHHC2, C2CD2, RMDN2, TMEM97, SLC19A3, VHL, SLC25A20, SEL1L3, UPK3BL2, IFI27, PTH2R, NEURL1, LRRC55, OBSCN, NUP54, VSIG10, ZC3H12A, TMPRSS2, SYCN, SLC39A2, SLC39A14, SLCO2B1, SLC4A4, RYK, REEP4, CRK, DPEP1, CUZD1, KIFC3, KIRREL2, FA2H, CDH6, GBA2, GPR34, GPAT2, GPRC5B, FES, LEPROTL1, AQP1, KDELR3, ACE2, CFTR, FXYD2, CAV2, PIK3AP1, AQP12A, AQP12B, IBTK, GRB14, OR10R2, NDUFB9,

TABLE 3C

List of proteins that meet criteria and have at least high expression on tissues where immune cell inhibition is desired (Ensembl version 92.38 ID for is provided for each gene symbol):
SELENBP1(ENSG00000143416), SMIM24(ENSG00000095932), RAB27B(ENSG00000041353), MUC1(ENSG00000185499),
SDC1(ENSG00000115884), TMEM45A(ENSG00000181458), PEX10(ENSG00000157911), CD46(ENSG00000117335),
TMEM236(ENSG00000148483), VPS35L(ENSG00000103544), TSPAN8(ENSG00000127324), SYTL2(ENSG00000137501),
SLC28A3(ENSG00000197506), PTAFR(ENSG00000169403), PIGZ(ENSG00000119227), SLC39A14(ENSG00000104635),
SLC6A15(ENSG00000072041), RAB25(ENSG00000132698), SLC20A2(ENSG00000168575), SPTLC3(ENSG00000172296),
MARK2(ENSG00000072518), CA2(ENSG00000104267), CDHR5(ENSG00000099834), CLDN4(ENSG00000189143),
DPEP1(ENSG0000001S413), ACSL5(ENSG00000197142), CLCA1(ENSG00000016490), DSG2(ENSG00000046604),
CEACAM5(ENSG00000105388), EPCAM(ENSG00000119888), BRI3(ENSG00000164713), CLDN8(ENSG00000156284),
GAL3ST2(ENSG00000154252), B3GNT8(ENSG00000177191), ELOVL7(ENSG00000164181), CLDN7(ENSG00000181885),
CDH17(ENSG00000079112), ATP11C(ENSG00000101974), C12orf66(ENSG00000174206), HHLA2(ENSG00000114455),
MAOA(ENSG00000189221), SLC9A3R2(ENSG00000065054), SLC2A6(ENSG00000160326), GPA33(ENSG00000143167),
MUC13(ENSG00000173702), SLC22A15(ENSG00000163393), SCEL(ENSG00000136155), SMURF1(ENSG00000198742),
AGER(ENSG00000204305), STEAP1(ENSG00000164647), CCR7(ENSG00000126353), CYB5A(ENSG00000166347),
FADS2(ENSG00000134824), LAMP3(ENSG00000078081), SLC34A2(ENSG00000157765), PERP(ENSG00000112378),
RAC1(ENSG00000136238), MTMR2(ENSG00000087053), TMEM134(ENSG00000172663), PROM2(ENSG00000155066),
KRT1(ENSG00000167768), STK16(ENSG00000115661), GJB4(ENSG00000189433), KCNJ8(ENSG00000121361),
DSP(ENSG00000096696), DSC3(ENSG00000134762), GSDMA(ENSG00000167914), DSG1(ENSG00000134760),
ADGRF4(ENSG00000153294), AQP3(ENSG00000165272), FGFR3(ENSG00000068078), PSKH1(ENSG00000159792),
SCEL(ENSG00000136155), SLC41A2(ENSG00000136052), ICAM3(ENSG00000076662), S100A8(ENSG00000143546),
DSC2(ENSG00000134755), FGFBP1(ENSG00000137440), OR10R2(ENSG00000198965), (), SELENBP1(ENSG00000143416),
SLCO1B3(ENSG00000111700), SLC25A43(ENSG00000077713), SLC27A5(ENSG00000083807), RDH16(ENSG00000139547),
TFR2(ENSG00000106327), RSAD2(ENSG00000134321), SLC30A10(ENSG00000196660), GHR(ENSG00000112964),
KCNJ8(ENSG00000121361), CYP2D6(ENSG00000100197), SLC2A2(ENSG00000163581), CYP2A7(ENSG00000198077),
CYP1A2(ENSG00000140505), CYP3A4(ENSG00000160868), CYP2C8(ENSG00000138115), CYP2C19(ENSG00000165841),
AIG1(ENSG00000146416), ASGR1(ENSG00000141505), ABCB11(ENSG00000073734), DCXR(ENSG00000169738),
CYP2C9(ENSG00000138109), ABCC2(ENSG00000023839), DPP4(ENSG00000197635), PODXL(ENSG00000128567),
PECAM1(ENSG00000261371), PLA2R1(ENSG00000153246), MAP6(ENSG00000171533), NPHS2(ENSG00000116218),
TNS2(ENSG00000111077), PTPRO(ENSG00000151490), EHD1(ENSG00000110047), CD34(ENSG00000174059),
ENG(ENSG00000106991), EHD3(ENSG00000013016), TRPM3(ENSG00000083067), VPS52(ENSG00000223501),
FCAMR(ENSG00000162897), SLC5A12(ENSG00000148942), SLC12A1(ENSG00000074803), SLC27A2(ENSG00000140284),
SLC6A19(ENSG00000174358), SLC22A11(ENSG00000168065), NFAM1(ENSG00000235568), PLLP(ENSG00000102934),
VHL(ENSG00000134086), PDZK1(ENSG00000174827), MGAT4B(ENSG00000161013), PROM2(ENSG00000155066),
NAT8(ENSG00000144035), UGT2B4(ENSG00000156096), SLC22A6(ENSG00000197901), TMEM72(ENSG00000187783),
SLC7A7(ENSG00000155465), UGT1A6(ENSG00000167165), TMEM174(ENSG00000164325), SLC28A1(ENSG00000156222),
RAB11FIP5(ENSG00000135631), SLC13A2(ENSG00000007216), SYT7(ENSG00000011347), SLC4A4(ENSG00000080493),
PKHD1(ENSG00000170927), ENPP6(ENSG00000164303), GGT1(ENSG00000100031), CUBN(ENSG00000107611),
HYAL2(ENSG00000068001), CDH6(ENSG00000113361), AQP6(ENSG00000086159), SLC7A9(ENSG00000021488),
FXYD2(ENSG00000137731), ACE(ENSG00000159640), AKAP10(ENSG00000108599), AQP3(ENSG00000165272),
CDH16(ENSG00000166589), INTS2(ENSG00000108506), MOXD1(ENSG00000079931), MC4R(ENSG00000166603),
LRP2(ENSG00000081479), OR10R2(ENSG00000198965), LRRK2(ENSG00000188906), NDST2(ENSG00000166507),
SLC27A6(ENSG00000113396), CD36(ENSG00000135218), RASL10B(ENSG00000270885), PARVB(ENSG00000188677),
PLN(ENSG00000198523), SGCG(ENSG00000102683), TMEM143(ENSG00000161558), PRRG2(ENSG00000126460),
DSP(ENSG00000096696), DSC2(ENSG00000134755), CAVIN4(ENSG00000170681), CHRNA1(ENSG00000138435),
CNTFR(ENSG00000122756), ARL4C(ENSG00000188042), MYZAP(ENSG00000263155), MSTO1(ENSG00000125459),
SLC16A10(ENSG00000112394), DES(ENSG00000175084), SLC5A5(ENSG00000105641), MPP7(ENSG00000150054),
TPO(ENSG00000115705), IFI27(ENSG00000165949), MUC15(ENSG00000169550), OLFM2(ENSG00000105088),
UNC13B(ENSG00000198722), IYD(ENSG00000009765), IL17RA(ENSG00000177663), SCGN(ENSG00000079689),
SCG3(ENSG00000104112), PTGDR2(ENSG00000183134), RAB3B(ENSG00000169213), PTPRN(ENSG00000054356),
MGAT4A(ENSG00000071073), SV2A(ENSG00000159164), YRDC(ENSG00000196449), TSPAN7(ENSG00000156298),
SLC30A8(ENSG00000164756), SYP(ENSG00000102003), PTPRN2(ENSG00000155093), DGCR2(ENSG00000070413),
GNAO1(ENSG00000087258), CASR(ENSG00000036828), ATP9A(ENSG00000054793), CADM1(ENSG00000182985),
TMEM97(ENSG00000109084), SGMS1(ENSG00000198964), SYCN(ENSG00000179751), CUZD1(ENSG00000138161),
GP2(ENSG00000169347), KIRREL2(ENSG00000126259), EVI2A(ENSG00000126860), AQP8(ENSG00000103375),
GPAT2(ENSG00000186281), LPAR6(ENSG00000139679), CFTR(ENSG00000001626), AQP12A(ENSG00000184945),
AQP12B(ENSG00000185176), UGT2A3(ENSG00000135220), CORO7(ENSG00000262246), PLEKHA3(ENSG00000116095),
UGT2B17(ENSG00000197888), SERAC1(ENSG00000122335), UGT2B28(ENSG00000135226), TMEM253(ENSG00000232070),
UGT2B15(ENSG00000196620), C2orf88(ENSG00000187699), FPR2(ENSG00000171049), HTR1A(ENSG00000178394),
CA12(ENSG00000074410), HSD3B7(ENSG00000099377), RPS6KC1(ENSG00000136643), SLC25A17(ENSG00000100372),
PSD4(ENSG00000125637), AOC3(ENSG00000131471), GLT8D2(ENSG00000120820), GBP2(ENSG00000162645),
AP3M1(ENSG00000185009), GJB4(ENSG00000189433), TNFSF12(ENSG00000239697), GBP2(ENSG00000162645),
SORD(ENSG00000140263), GATM(ENSG00000171766), GCHFR(ENSG00000137880), FMO3(ENSG00000007933),
ABCB4(ENSG00000005471), BCL2L10(ENSG00000137875), UGCG(ENSG00000148154), HSD11B1(ENSG00000117594),
KDR(ENSG00000128052), KIRREL1(ENSG00000183853), PTH2R(ENSG00000144407), MME(ENSG00000196549),
SNCA(ENSG00000145335), RYK(ENSG00000163785), SLC35B1(ENSG00000121073), CLSTN1(ENSG00000171603),
ENPEP(ENSG00000138792), ITGA8(ENSG00000077943), SLC34A3(ENSG00000198569), PALM(ENSG00000099864),
XPNPEP2(ENSG00000122121), SLC13A3(ENSG00000158296), ADCY7(ENSG00000121281), FAM151A(ENSG00000162391),
CHDH(ENSG00000016391), FAM210A(ENSG00000177150), L1CAM(ENSG00000198910), BSND(ENSG00000162399),
AQP1(ENSG00000240583), SMPD4(ENSG00000136699), CAP2(ENSG00000112186), GABARAPL2(ENSG00000034713),
TNFSF12(ENSG00000239697), SGCB(ENSG00000163069), BVES(ENSG00000112276), CKMT2(ENSG00000131730),
SLC2A4(ENSG00000181856), CPT1B(ENSG00000205560), COX7B(ENSG00000131174), MLIP(ENSG00000146147),
SHISAL2B(ENSG00000145642), CD99(ENSG00000002586), DIAPH1(ENSG00000131504), CADPS(ENSG00000163618),
FKBP2(ENSG00000173486), ATP8B2(ENSG00000143515), HRAS(ENSG00000174775), RHBDL2(ENSG00000158315),
KRAS(ENSG00000133703), NRAS(ENSG00000213281), STXBP3(ENSG00000116266), NECTIN1(ENSG00000110400),
SLC9A1(ENSG00000090020), SNTA1(ENSG00000101400), PCDH1(ENSG00000156453), RAB27A(ENSG00000069974),
EMC9(ENSG00000100908), AVEN(ENSG00000169857), B3GNT7(ENSG00000156966), MARCKSL1(ENSG00000175130),
GPER1(ENSG00000164850), HCN3(ENSG00000143630), MUC4(ENSG00000145113), NAPEPLD(ENSG00000161048),
GPC5(ENSG00000179399), LYPD3(ENSG00000124466), SRC(ENSG00000197122), PARD3B(ENSG00000116117),
LY6D(ENSG00000167656), FAM83B(ENSG00000168143), FRS3(ENSG00000137218), EPHA10(ENSG00000183317),
ORAI1(ENSG00000276045), NISCH(ENSG00000010322), MARCKSL1(ENSG00000175130), TMEM243(ENSG00000135185),
NECTIN1(ENSG00000110400), S100A9(ENSG00000163220), FAT2(ENSG00000086570), SLC25A20(ENSG00000178537),
GNPNAT1(ENSG00000100522), TM4SF4(ENSG00000169903), CYP4F3(ENSG00000186529), EPHX1(ENSG00000143819),
CYP4F11(ENSG00000171903), CYP2A6(ENSG00000255974), ATP5F1C(ENSG00000165629), CYP4F2(ENSG00000186115),
CYP2A13(ENSG00000197838), CYP2E1(ENSG00000130649), GIPC1(ENSG00000123159), ST3GAL6(ENSG00000064225),
IL17RE(ENSG00000163701), STRA6(ENSG00000137868), VPS26B(ENSG00000151502), MITD1(ENSG00000158411),
LRRC55(ENSG00000183908), PARD3B(ENSG00000116117), SLC23A1(ENSG00000170482), SLC7A8(ENSG00000092068),
FERMT1(ENSG00000101311), TMCC1(ENSG00000172765), PPM1L(ENSG00000163590), CYP2W1(ENSG00000073067),
FAM168B(ENSG00000152102), KPNA2(ENSG00000182481), IL17RC(ENSG00000163702), ATP2A2(ENSG00000174437),
NISCH(ENSG00000010322), MBTPS1(ENSG00000140943), LPCAT2(ENSG00000087253), KCNQ1(ENSG00000053918),
SLC8A1(ENSG00000183023), CDH5(ENSG00000179776), STX5(ENSG00000162236), SLC12A6(ENSG00000140199),
‘KCTD3(ENSG00000136636), TMEM60(ENSG00000135211), RNF144B(ENSG00000137393), ABCG8(ENSG00000143921),
HPS6(ENSG00000166189), CRK(ENSG00000167193), CYP2B6(ENSG00000197408), ANPEP(ENSG00000166825),
ILK(ENSG00000166333), AIF1L(ENSG00000126878), NIPAL4(ENSG00000172548), DYSF(ENSG00000135636),
SLC47A1(ENSG00000142494), ATP6V1D(ENSG00000100554), SLC16A14(ENSG00000163053), TMEM219(ENSG00000149932),
AKR1A1(ENSG00000117448), ATRAID(ENSG00000138085), CLIC4(ENSG00000169504), ANK2(ENSG00000145362),
MYORG(ENSG00000164976), PAM(ENSG00000145730), ENPP1(ENSG00000197594), RAB43(ENSG00000172780),
BCL2(ENSG00000171791), MXRA7(ENSG00000182534), BMPR1A(ENSG00000107779), ZDHHC2(ENSG00000104219),
SLC30A7(ENSG00000162695), SRI(ENSG00000075142), SLC26A2(ENSG00000155850), SFXN5(ENSG00000144040),
PIGR(ENSG00000162896), TULP3(ENSG00000078246), TJP3(ENSG00000105289), TCIRG1(ENSG00000110719),
RAP1GAP(ENSG00000076864), ABCC3(ENSG00000108846), GPR183(ENSG00000169508), SLC24A3(ENSG00000185052),
MGST2(ENSG00000085871), TNFAIP8L3(ENSG00000183578), VPS28(ENSG00000160948), TMEM201(ENSG00000188807),
TRPM4(ENSG00000130529), FCER1G(ENSG00000158869), CLN6(ENSG00000128973), GALNT16(ENSG00000100626),
ABCD3(ENSG00000117528), NBAS(ENSG00000151779), MFSD10(ENSG00000109736), VTI1B(ENSG00000100568),
TMEM164(ENSG00000157600), CYP20A1(ENSG00000119004), GALNT16(ENSG00000100626), MERTK(ENSG00000153208),
ELOVL1(ENSG00000066322), COL13A1(ENSG00000197467), PIGU(ENSG00000101464), GPC4(ENSG00000076716),
EVA1A(ENSG00000115363), FXYD6(ENSG00000137726), CDH2(ENSG00000170558), GK(ENSG00000198814),
ACER3(ENSG00000078124), PLPP1(ENSG00000067113), GJA4(ENSG00000187513), CACNA1C(ENSG00000151067),
TTC17(ENSG00000052841), ACY3(ENSG00000132744), RNF144A(ENSG00000151692), RDX(ENSG00000137710),
SEL1L3(ENSG00000091490), UCHL1(ENSG00000154277), SCARA3(ENSG00000168077), SLC3A1(ENSG00000138079),
TNFRSF14(ENSG00000157873), TMPRSS2(ENSG00000184012), IMMT(ENSG00000132305), ADCK2(ENSG00000133597),
SLC48A1(ENSG00000211584), TESC(ENSG00000088992), ELOVL1(ENSG00000066322), SLC37A4(ENSG00000137700),
TMEM63B(ENSG00000137216), MT-ND4(ENSG00000198886), NDUFS8(ENSG00000110717), SUSD6(ENSG00000100647),
SLC2A10(ENSG00000197496), APOOL(ENSG00000155008), ICA1(ENSG00000003147), TPCN1(ENSG00000186815),
ANKRD13A(ENSG00000076513), SERP1(ENSG00000120742), TNFSF14(ENSG00000125735), MERTK(ENSG00000153208),
FAR2(ENSG00000064763), SLC2A11(ENSG00000133460), PDLIM4(ENSG00000131435), TMEM43(ENSG00000170876),
CAV1(ENSG00000105974), MTMR2(ENSG00000087053), CLCA2(ENSG00000137975), ADGRA3(ENSG00000152990),
GJA1(ENSG00000152661), CAV1(ENSG00000105974), GAA(ENSG00000171298), ZDHHC6(ENSG00000023041),
VIPR2(ENSG00000106018), KCNJ1(ENSG00000151704), EFHD1(ENSG00000115468), GJA1(ENSG00000152661),
TRPV4(ENSG00000111199), TMEM263(ENSG00000151135), MARCKS(ENSG00000277443), AKAP12(ENSG00000131016),
PPP1R3A(ENSG00000154415), SNX1(ENSG00000028528), ICAM1(ENSG00000090339), SNX1(ENSG00000028528),
TMEM245(ENSG00000106771), CLEC2B(ENSG00000110852), MFN2(ENSG00000116688), GNAI1(ENSG00000127955),
TEX2(ENSG00000136478), SMPD2(ENSG00000135587), UTRN(ENSG00000152818), LRRC24(ENSG00000254402),
MGLL(ENSG00000074416), KCNMB3(ENSG00000171121), APOBR(ENSG00000184730), ATP8B1(ENSG00000081923),
MRAP(ENSG00000170262), PAG1(ENSG00000076641), APOBR(ENSG00000184730), SLC38A9(ENSG00000177058),
SPIRE1(ENSG00000134278), HHAT(ENSG00000054392), NPHS1(ENSG00000161270), VAT1(ENSG00000108828),
ITPR2(ENSG00000123104), DAB2(ENSG00000153071), ACVRL1(ENSG00000139567), TMEM37(ENSG00000171227),
LFNG(ENSG00000106003), NDUFB9(ENSG00000147684), PVR(ENSG00000073008), FXYD7(ENSG00000221946),
MFSD11(ENSG00000092931), FKBP11(ENSG00000134285), TMED6(ENSG00000157315), SLC38A5(ENSG00000017483),
SMAGP(ENSG00000170545), TMEM94(ENSG00000177728), PLXNA1(ENSG00000114554), SLC25A46(ENSG00000164209),
TFPI(ENSG00000003436), LYPLA1(ENSG00000120992), NUCB2(ENSG00000070081), VPS11(ENSG00000160695),
SIGIRR(ENSG00000185187), TECPR1(ENSG00000205356), VPS41(ENSG00000006715), CLMN(ENSG00000165959),
SLC25A13(ENSG00000004864), ANKRD46(ENSG00000186106), CDH26(ENSG00000124215), ADGRL1(ENSG00000072071),
RAB20(ENSG00000139832), C2CD2(ENSG00000157617), NAGPA(ENSG00000103174), KCNK1(ENSG00000135750),
TMEM159(ENSG00000011638), ATP6V1B1(ENSG00000116039), TRPV5(ENSG00000127412), CACNA1B(ENSG00000148408),
COQ2(ENSG00000173085), COX14(ENSG00000178449), SLC2A5(ENSG00000142583), CPM(ENSG00000135678),
SLC16A4(ENSG00000168679), VDAC3(ENSG00000078668), DOK7(ENSG00000175920), TSPAN3(ENSG00000140391),
HEG1(ENSG00000173706), HLA-DQB1(ENSG00000179344), NBEA(ENSG00000172915), VAMP1(ENSG00000139190),
SRPRB(ENSG00000144867), KIFC3(ENSG00000140859), TMEM106C(ENSG00000134291), TSNARE1(ENSG00000171045),
FAM162A(ENSG00000114023), PYCARD(ENSG00000103490), SDR16C5(ENSG00000170786), CAV2(ENSG00000105971),
PPP1R16B(ENSG00000101445), SDR16C5(ENSG00000170786), TSNARE1(ENSG00000171045), CAV2(ENSG00000105971),
PYCARD(ENSG00000103490), PLPPR2(ENSG00000105520), PRKN(ENSG00000185345), TOMM40L(ENSG00000158882),
TMX2(ENSG00000213593), MAP1LC3B(ENSG00000140941), PPP1R16B(ENSG00000101445), PLOD1(ENSG00000083444), SDCBP2(ENSG00000125775), GBA2(ENSG00000070610), EPB41L2(ENSG00000079819), FGFR2(ENSG00000066468),
RETSAT(ENSG00000042445)

### List of All Proteins in the List Above Separately Categorized by Applicable Use

(Ensembl version 92.38 ID for the symbols below can be found in the table above):
1. Cancer treated: melanoma
   a. Tissue protected: Colon SELENBP1, SMIM24, RAB27B, MUC1, SDC1, TMEM45A, PEX10, CD46, TMEM236, VPS35L, TSPAN8, SYTL2, SLC28A3, PTAFR, PIGZ, SLC39A14, SLC6A15, RAB25, SLC20A2, SPTLC3, MARK2, CA2, CDHR5, CLDN4, DPEP1, ACSLS, CLCA1, DSG2, CEACAM5, EPCAM, BRI3, CLDN8, GAL3ST2, B3GNT8, ELOVL7, CLDN7, CDH17, ATP11C, C12orf66, HHLA2, MAOA, SLC9A3R2, SLC2A6, GPA33, MUC13,
   b. Tissue protected: Lung SLC22A15, SCEL, SMURF1, AGER, SLC6A15, RAB25, STEAP1, CCR7, CYB5A, FADS2, MAOA, LAMP3, SLC34A2,
   c. Tissue protected: Skin PERP, RAC1, MTMR2, TMEM134, PROM2, KRT1, STK16, GJB4, KCNJ8, DSP, DSC3, GSDMA, DSG1, ADGRF4, AQP3, FGFR3, PSKH1, SCEL, SLC41A2, ICAM3, S100A8, DSC2, FGFBP1, OR10R2,
   d. Tissue protected: Liver SELENBP1, SLCO1B3, SLC25A43, SLC27A5, RDH16, TFR2, RSAD2, SLC30A10, GHR, KCNJ8, CYP2D6, ACSLS, SLC2A2, CYP2A7, CYP1A2, CYP3A4, CYP2C8, CYP2C19, FADS2, AIG1, ASGR1, ABCB11, DCXR, CYP2C9, ABCC2, DPP4,
   e. Tissue protected: Kidney (glomeruli) PODXL, PECAM1, PLA2R1, MAP6, NPHS2, TNS2, PTPRO, EHD1, CD34, ENG, SLC9A3R2, EHD3,
   f. Tissue protected: Kidney (tubules) TRPM3, VPS52, FCAMR, SMIM24, SLC5A12, SLC12A1, SLC27A2, SLC6A19, SLC22A11, NFAM1, PLLP, VHL, RAB27B, PDZK1, SDC1, MGAT4B, PROM2, NAT8, UGT2B4, SLC22A6, TMEM72, SLC7A7, UGT1A6, TMEM174, SYTL2, SLC28A1, PTAFR, PIGZ, RAB25, RAB11FIP5, SLC13A2, SYT7, SLC4A4, MARK2, PKHD1, CA2, CDHR5, ENPP6, GGT1, DPEP1, CUBN, HYAL2, KCNJ8, ACSLS, CDH6, AQP6, CLDN8, CLDN7, SLC7A9, FXYD2, ACE, DCXR, AKAP10, AQP3, CDH16, INTS2, MAOA, MOXD1, MC4R, SLC2A6, LRP2, OR10R2, ABCC2, DPP4, LRRK2, NDST2,
   g. Tissue protected: Heart (cardiomyocytes) SLC27A6, CD36, RASL10B, SLC22A15, NFAM1, PARVB, PLN, SGCG, TMEM143, PRRG2, DSP, DSC2, CAVIN4, DSG2, CHRNA1, CNTFR, ARL4C, MYZAP, MSTO1, SLC16A10, OR10R2, DES,
   h. Tissue protected: Thyroid SELENBP1, SLC5A5, MPP7, TPO, IFI27, MUC15, OLFM2, UNC13B, RAB25, IYD, CLDN7, CDH16, MAOA, MSTO1, IL17RA,
   i. Tissue protected: Pancreatic endocrine cells (Islets) SCGN, SCG3, PTGDR2, RAB3B, PTPRN, MGAT4A, SV2A, YRDC, TSPAN7, SLC30A8, SYP, PTPRN2, RAB25, DGCR2, GNAO1, CASR, CLDN7, ATP9A, CADM1, MAOA,
   j. Tissue protected: Pancreatic exocrine cells VPS52, RASL10B, TMEM97, IFI27, SGMS1, OLFM2, YRDC, SYCN, RSAD2, RAB25, SLC20A2, SLC4A4, CLDN4, DPEP1, CUZD1, GP2, KIRREL2, EVI2A, AQP8, GPAT2, LPAR6, FADS2, CLDN8, ELOVL7, CLDN7, CFTR, FXYD2, MAOA, AQP12A, AQP12B, MOXD1, MC4R, IL17RA,
2. Cancer treated: lung cancer
   a. Tissue protected: Colon UGT2A3, CORO7, SMIM24, PLEKHA3, TMEM45A, PEX10, UGT2B17, SERAC1, UGT2B28, TMEM253, UGT2B15, C2orf88, SLC39A14, CA2, DPEP1, CLCA1, FPR2, HTR1A, CA12, BRI3, GAL3ST2, ELOVL7, HSD3B7, SLC2A6, RPS6KC1,
   b. Tissue protected: Lung SLC25A17, AGER, PSD4, CCR7, AOC3, GLT8D2, GBP2, AP3M1,
   c. Tissue protected: Skin GJB4, DSP, GSDMA, PSKH1, TNFSF12, GBP2, OR10R2,
   d. Tissue protected: Liver SLCO1B3, SLC27A5, SORD, TFR2, CYP2D6, SLC2A2, CYP2A7, GLT8D2, GATM, CYP1A2, GCHFR, CYP3A4, FMO3, ABCB4, CYP2C8, CYP2C19, BCL2L10, UGCG, ASGR1, ABCB11, HSD11B1, CYP2C9, ABCC2,
   e. Tissue protected: Kidney (glomeruli) KDR, PODXL, KIRREL1, PECAM1, PLA2R1, PTH2R, MME, NPHS2, TNS2, SNCA, RYK, SLC35B1, CLSTN1, EHD1, CD34, GLT8D2, ENG, ENPEP, ITGA8, EHD3,
   f. Tissue protected: Kidney (tubules) TRPM3, FCAMR, SMIM24, SLC5A12, SLC12A1, SLC27A2, SLC6A19, SLC34A3, NAT8, PALM, MME, UGT2B4, SLC22A6, TMEM72, XPNPEP2, SLC7A7, SERAC1, TMEM174, SLC28A1, SLC13A3, RAB11FIP5, SLC13A2, SYT7, SLC4A4, PKHD1, CA2, ENPP6, GGT1, DPEP1, CUBN, HYAL2, ADCY7, FAM151A, CHDH, HTR1A, GLT8D2, GATM, FAM210A, CA12, L1CAM, BSND, BCL2L10, AQP6, AQP1, UGCG, ENPEP, SLC7A9, FXYD2, ACE, AKAP10, HSD3B7, CDH16, INTS2, SMPD4, SLC2A6, LRP2, OR10R2, ABCC2, LRRK2,
   g. Tissue protected: Heart (cardiomyocytes) CAP2, CD36, GABARAPL2, RASL10B, TNFSF12, SGCB, BVES, PLN, SGCG, SERAC1, CKMT2, DSP, CAVIN4, SLC2A4, GLT8D2, CHRNA1, GBP2, CPT1B, COX7B, MYZAP, MLIP, OR10R2, DES,
   h. Tissue protected: Thyroid SORD, IYD, CLSTN1, CDH16,
   i. Tissue protected: Pancreatic endocrine cells (Islets) SCGN, SCG3, PTGDR2, RAB3B, PTPRN, SHISAL2B, YRDC, TSPAN7, SLC30A8, PTPRN2, DGCR2, CASR, CD99, DIAPH1, GBP2, CADPS,
   j. Tissue protected: Pancreatic exocrine cells RASL10B, TMEM97, PLEKHA3, PTH2R, YRDC, SYCN, SLC4A4, DPEP1, CUZD1, KIRREL2, ADCY7, FKBP2, EVI2A, GATM, AQP8, DIAPH1, GPAT2, CA12, GBP2, AQP1, ELOVL7, CFTR, FXYD2, AQP12A, AQP12B,
3. Cancer treated: renal cancer
   a. Tissue protected: Colon UGT2A3, ATP8B2, SMIM24, HRAS, RHBDL2, KRAS, TMEM45A, NRAS, STXBP3, PEX10, CD46, NECTIN1, SLC9A1, SNTA1, UGT2B17, TMEM236, VPS35L, UGT2B28, TMEM253, TSPAN8, UGT2B15, PCDH1, SLC28A3, RAB27A, PIGZ, CA2, DPEP1, EMC9, CLCA1, CEACAM5, BRI3, GAL3ST2, CDH17, AVEN, B3GNT7, SLC9A3R2, MARCKSL1, GPER1, HCN3, GPA33, RPS6KC1, MUC13, MUC4, NAPEPLD,
   b. Tissue protected: Lung NECTIN1, PSD4, STEAP1, CCR7, GPC5, LAMP3,
   c. Tissue protected: Skin PERP, RAC1, MTMR2, TMEM134, PROM2, LYPD3, KRT1, SRC, PARD3B, LY6D, FAM83B, GJB4, DSP, FRS3, DSC3, GSDMA, EPHA10, DSG1, ORAI1, NISCH, MARCKSL1, FGFR3, TMEM243, SLC41A2, ICAM3, NECTIN1, S100A9, FAT2, FGFBP1, OR10R2,
   d. Tissue protected: Liver SLCO1B3, SLC27A5, SLC25A20, RDH16, GNPNAT1, SORD, TFR2, TM4SF4, SLC30A10, CYP2D6, CYP4F3, CYP2A7, EPHX1, CYP4F11, CYP1A2, CYP3A4, CYP2A6, CYP2C8, CYP2C19, ATP5F1C, CYP4F2, ASGR1, ABCB11, CYP2A13, CYP2E1, DCXR, HSD11B1, CYP2C9, GIPC1, ABCC2,
   e. Tissue protected: Kidney (glomeruli) ST3GAL6, KDR, PODXL, PECAM1, PLA2R1, PTH2R, MAP6, NPHS2, TNS2, SNCA, PTPRO, EHD1, CD34, ENG, SLC9A3R2, IL17RE, ITGA8, EHD3,
   f. Tissue protected: Kidney (tubules) TRPM3, STRA6, SMIM24, SLC12A1, SLC34A3, VPS26B, MGAT4B, MITD1, LRRC55, PROM2, PALM, SLC7A7, TMEM174, PARD3B, PIGZ, SLC13A2, SLC4A4, SLC23A1, PKHD1, CA2, ENPP6, DPEP1, ADCY7, FAM210A, SLC7A8, BSND, AQP6, ATP5F1C, CYP4F2, FERMT1, ACE, DCXR, SMPD4, GIPC1, OR10R2, ABCC2, NAPEPLD,
   g. Tissue protected: Heart (cardiomyocytes) TMCC1, CD36, RASL10B, PARVB, PLN, SGCG, TMEM143, PPM1L, CYP2W1, DSP, CAVIN4, SLC2A4, FAM168B, CHRNA1, KPNA2, IL17RC, ATP2A2, CPT1B, COX7B, ARL4C, NISCH, MYZAP, MBTPS1, MLIP, OR10R2, DES,
   h. Tissue protected: Thyroid LPCAT2, MPP7, TPO, IFI27, MUC15, OLFM2, LRRC55, KCNQ1, SLC8A1, SORD, IYD, EMC9, CYP2W1, IL17RC, GIPC1, MARCKSL1, IL17RA,
   i. Tissue protected: Pancreatic endocrine cells (Islets) SCG3, PTGDR2, RAB3B, PTPRN, SV2A, YRDC, TSPAN7, SLC30A8, SYP, PTPRN2, DGCR2, CYP2W1, GNAO1, CASR, CD99, CDH5, CADPS,
   j. Tissue protected: Pancreatic exocrine cells RASL10B, TMEM97, STX5, IFI27, STXBP3, MITD1, GNPNAT1, PTH2R, OLFM2, NECTIN1, YRDC, SYCN, SLC12A6, SLC4A4, DPEP1, EMC9, GP2, KCTD3, ADCY7, EVI2A, AQP8, GPAT2, CFTR, AVEN, AQP12A, AQP12B, IL17RA,
4. Cancer treated: urothelial cancer
   a. Tissue protected: Colon UGT2A3, TMEM60, SMIM24, RNF144B, STXBP3, PEX10, UGT2B17, TMEM236, UGT2B28, TMEM253, UGT2B15, RAB27A, CA2, DPEP1, CLCA1, FPR2, HTR1A, ABCG8, GAL3ST2, B3GNT8, ELOVL7, CDH17, C12orf66, GPA33, RPS6KC1, MUC13, HPS6,
   b. Tissue protected: Lung SLC22A15, SLC25A17, PSD4, STEAP1, CCR7, AOC3, LAMP3,
   c. Tissue protected: Skin PERP, DSG1, OR10R2,
   d. Tissue protected: Liver SLCO1B3, SLC27A5, RNF144B, TFR2, TM4SF4, CRK, CYP2D6, SLC2A2, CYP2A7, CYP1A2, CYP3A4, CYP2A6, CYP2B6, FMO3, CYP2C8, CYP2C19, BCL2L10, ANPEP, ASGR1, CYP2A13, HSD11B1, CYP2C9, HPS6, ABCC2, DPP4,
   e. Tissue protected: Kidney (glomeruli) ILK, KIRREL1, PLA2R1, PTH2R, MAP6, NPHS2, TNS2, SNCA, PTPRO, EHD1, AIF1L, CD34, ENG, ITGA8, EHD3, NIPAL4, HPS6, DYSF,
   f. Tissue protected: Kidney (tubules) TRPM3, STRA6, SLC47A1, SMIM24, SLC5A12, SLC12A1, SLC27A2, SLC34A3, VPS26B, RNF144B, MITD1, NAT8, UGT2B4, ATP6V1D, SLC22A6, TMEM72, SLC7A7, SLC16A14, TMEM174, SLC28A1, SLC13A3, SLC13A2, SLC4A4, PKHD1, TMEM219, CRK, CA2, GGT1, DPEP1, CUBN, AIF1L, CDH6, FAM151A, CHDH, HTR1A, AKR1A1, ATRAID, CLIC4, BCL2L10, AQP1, ANK2, ANPEP, SLC7A9, FXYD2, ACE, CDH16, INTS2, LRP2, HPS6, OR10R2, ABCC2, DPP4, LRRK2,
   g. Tissue protected: Heart (cardiomyocytes) CD36, SLC22A15, BVES, PARVB, PLN, SGCG, MYORG, PRRG2, CKMT2, CYP2W1, CAVIN4, CHRNA1, CPT1B, PAM, MYZAP, OR10R2, DES,
   h. Tissue protected: Thyroid ENPP1, TPO, IFI27, SLC16A14, UNC13B, RAB43, IYD, CYP2W1, ABCG8, BCL2, CDH16,
   i. Tissue protected: Pancreatic endocrine cells (Islets) SCGN, SCG3, RAB3B, MXRA7, PTPRN, SHISAL2B, SV2A, YRDC, TSPAN7, SLC30A8, PTPRN2, DGCR2, CYP2W1, GNAO1, CADPS, CADM1,
   j. Tissue protected: Pancreatic exocrine cells IFI27, STXBP3, MITD1, PTH2R, YRDC, SYCN, SLC4A4, DPEP1, CUZD1, GP2, KIRREL2, BMPR1A, FKBP2, GPAT2, AQP1, ELOVL7, ANPEP, CFTR, FXYD2, AQP12A, AQP12B, HPS6,
5. Cancer treated: head and neck cancer
   a. Tissue protected: Colon ZDHHC2, SLC30A7, UGT2A3, ATP8B2, CORO7, SMIM24, SRI, SLC26A2, SFXN5, PIGR, PLEKHA3, TULP3, TJP3, TCIRG1, RAP1GAP, TMEM45A, PEX10, ABCC3, CD46, GPR183, SLC24A3, MGST2, TNFAIP8L3, UGT2B17, TMEM236, VPS28, VPS35L, UGT2B28, TMEM253, TSPAN8, SYTL2, TMEM201, UGT2B15, PCDH1, SLC28A3, PTAFR, RAB27A, PIGZ, SLC39A14, SLC6A15, TRPM4, CA2, CDHR5, DPEP1, FCER1G, ACSL5, CLCA1, FPR2, HTR1A, CLN6, GALNT16, BRI3, B3GNT8, ELOVL7, CDH17, ATP11C, B3GNT7, HSD3B7, ABCD3, HHLA2, SLC9A3R2, NBAS, SLC2A6, GPA33, MUC13, HPS6, MUC4,
   b. Tissue protected: Lung AGER, MFSD10, PSD4, SLC6A15, VTI1B, TMEM164, STEAP1, CYB5A, AOC3, AP3M1, CYP20A1, ABCD3, LAMP3, SLC34A2,
   c. Tissue protected: Skin PERP, GSDMA, GALNT16, ADGRF4, PSKH1, TMEM243, ICAM3, MERTK, FAT2, ELOVL1, FGFBP1, COL13A1, OR10R2,
   d. Tissue protected: Liver SLCO1B3, SLC25A43, SLC27A5, PIGU, RDH16, MGST2, SORD, TFR2, TMEM201, GPC4, EVA1A, GHR, CYP2D6, ACSL5, SLC2A2, CYP2A7, CYP1A2, FXYD6, CYP3A4, CYP2A6, FMO3, ABCB4, CYP2C8, CDH2, CYP2C19, AIG1, UGCG, ANPEP, ASGR1, ABCB11, CYP2A13, CYP2E1, HSD11B1, ABCD3, CYP2C9, NBAS, GIPC1, GK, HPS6, ABCC2,
   e. Tissue protected: Kidney (glomeruli) ACER3, ST3GAL6, PLPP1, PECAM1, PLA2R1, PTH2R, MAP6, NPHS2, TNS2, PTPRO, SLC35B1, AIF1L, CD34, ENG, GJA4, ENPEP, CACNA1C, SLC9A3R2, ITGA8, NIPAL4, HPS6, DYSF,
   f. Tissue protected: Kidney (tubules) TRPM3, TTC17, ACY3, FCAMR, SLC47A1, SMIM24, SLC5A12, SLC12A1, RNF144A, RDX, SLC6A19, SLC22A11, SLC34A3, PIGR, VPS26B, PIGU, PDZK1, SEL1L3, UCHL1, SCARA3, GPR183, LRRC55, NAT8, SLC24A3, UGT2B4, ATP6V1D, SLC22A6, TMEM72, TNFAIP8L3, XPNPEP2, SLC3A1, SLC7A7, UGT1A6, TMEM174, TNFRSF14, SYTL2, TMPRSS2, SLC28A1, PTAFR, SLC13A3, PIGZ, SLC13A2, VTI1B, SLC4A4, PKHD1, IMMT, TMEM219, ADCK2, CA2, CDHR5, ENPP6, GGT1, DPEP1, CUBN, GPC4, HYAL2, SLC48A1, AIF1L, ACSLS, CDH6, FAM151A, CHDH, HTR1A, TESC, CLN6, ATRAID, FXYD6, CLIC4, FAM210A, ELOVL1, SLC37A4, CDH2, BSND, AQP6, AQP1, UGCG, ANK2, ANPEP, ENPEP, SLC7A9, FXYD2, ACE, TMEM63B, HSD3B7, CDH16, ABCD3, INTS2, SMPD4, MOXD1, NBAS, GIPC1, MC4R, SLC2A6, LRP2, HPS6, MT-ND4, OR10R2, ABCC2, LRRK2,
   g. Tissue protected: Heart (cardiomyocytes) SLC27A6, CAP2, GABARAPL2, RASL10B, RNF144A, SFXN5, SGCB, NDUFS8, BVES, PARVB, PLN, RAP1GAP, SGCG, GPR183, TMEM143, SUSD6, VTI1B, TMEM164, PPM1L, IMMT, CKMT2, CYP2W1, CAVIN4, SLC2A4, CHRNA1, CNTFR, CDH2, CPT1B, PAM, ABCD3, MYZAP, NBAS, SLC2A10, MLIP, APOOL, SLC16A10, ICA1, OR10R2, DES,
   h. Tissue protected: Thyroid SFXN5, TPO, TPCN1, TCIRG1, RAP1GAP, IFI27, LRRC55, SLC24A3, SORD, VTI1B, IYD, GPC4, CYP2W1, ANKRD13A, BCL2, CYP20A1, CDH16, NBAS, GIPC1, IL17RA,
   i. Tissue protected: Pancreatic endocrine cells (Islets) ZDHHC2, TTC17, SCGN, SCG3, PTGDR2, RAB3B, UCHL1, SERP1, PTPRN, MGAT4A, SHISAL2B, SV2A, YRDC, TSPAN7, SLC30A8, SYP, PTPRN2, VTI1B, DGCR2, CYP2W1, GNAO1, CASR, CD99, ATP9A, CADM1,
   j. Tissue protected: Pancreatic exocrine cells TNFSF14, RASL10B, SFXN5, TMEM97, PLEKHA3, RAP1GAP, IFI27, PTH2R, MERTK, YRDC, SYCN, VTI1B, SLC4A4, DPEP1, CUZD1, GPC4, GP2, KIRREL2, FAR2, TESC, AQP8, GPAT2, LPAR6, AQP1, ELOVL7, ANPEP, CFTR, FXYD2, CYP20A1, AQP12A, AQP12B, MOXD1, NBAS, MC4R, APOOL, IL17RA, HPS6, SLC2A11, PDLIM4,
6. Cancer treated: colorectal cancer
   a. Tissue protected: Colon RAB27B, TMEM253, CA2, SLC9A3R2,
   b. Tissue protected: Lung SLC22A15, SLC25A17, AGER, TMEM43, PSD4, STEAP1, CCR7, CYB5A, GPC5, AOC3, CAV1, LAMP3, SLC34A2,
   c. Tissue protected: Skin MTMR2, LYPD3, KRT1, CLCA2, GJB4, ADGRA3, GJA1, GSDMA, DSG1, ADGRF4, CAV1, TMEM243, MTMR2, FAT2, OR10R2,
   d. Tissue protected: Liver SLCO1B3, SLC27A5, RDH16, TFR2, EVA1A, CYP2D6, SLC2A2, CYP2A7, GATM, EPHX1, CYP1A2, CYP3A4, CYP2A6, CYP2B6, FMO3, CYP2C8, CDH2, CYP2C19, BCL2L10, AIG1, ANPEP, ASGR1, ABCB11, CYP2A13, DCXR, CYP2C9, GK, GAA,
   e. Tissue protected: Kidney (glomeruli) ZDHHC6, ACER3, ST3GAL6, VIPR2, KDR, PODXL, KIRREL1, PECAM1, PLA2R1, PTH2R, NPHS2, TNS2, SNCA, RYK, PTPRO, SLC35B1, EHD1, AIF1L, CD34, ENG, SLC9A3R2, ITGA8, EHD3, DYSF,
   f. Tissue protected: Kidney (tubules) TRPM3, SLC5A12, SLC12A1, RNF144A, SLC27A2, SLC6A19, SLC34A3, RAB27B, KCNJ1, UCHL1, LRRC55, NAT8, UGT2B4, ATP6V1D, SLC22A6, TMEM72, SLC7A7, TMEM174, SLC28A1, SLC13A2, SLC4A4, SLC23A1, CA2, ENPP6, EFHD1, CUBN, AIF1L, CDH6, FAM151A, GATM, ATRAID, CLIC4, FAM210A, CDH2, BSND, BCL2L10, ANPEP, FXYD2, ACE, TMEM63B, DCXR, CDH16, LRP2, OR10R2, LRRK2,
   g. Tissue protected: Heart (cardiomyocytes) SLC27A6, CAP2, CD36, RASL10B, SLC22A15, RNF144A, SGCB, BVES, PLN, SGCG, PRRG2, FAM168B, CHRNA1, GJA1, CDH2, CPT1B, COX7B, MYZAP, OR10R2, DES,
   h. Tissue protected: Thyroid ZDHHC6, ENPP1, TPO, LRRC55, IYD, CDH16, GAA,
   i. Tissue protected: Pancreatic endocrine cells (Islets) SCGN, TRPV4, SCG3, PTGDR2, RAB3B, UCHL1, PTPRN, SV2A, YRDC, TSPAN7, SLC30A8, SYP, DGCR2, GNAO1, CASR, CD99, CADPS,
   j. Tissue protected: Pancreatic exocrine cells RASL10B, TMEM263, TMEM97, PTH2R, YRDC, SYCN, SLC4A4, CUZD1, KIRREL2, GATM, GPAT2, ANPEP, FXYD2, AQP12A, AQP12B, SLC2A11, GAA,
7. Cancer treated: liver cancer
   a. Tissue protected: Colon ZDHHC2, SLC26A2, RAB27A, DPEP1, CLCA1, EPCAM, GAL3ST2, C12orf66, MARCKS, MUC13,
   b. Tissue protected: Lung SLC22A15, PSD4, STEAP1, CCR7, GPC5, LAMP3,
   c. Tissue protected: Skin PERP, MTMR2, LYPD3, CLCA2, DSP, GSDMA, DSG1, AQP3, TMEM243, S100A8, FAT2, OR10R2,
   d. Tissue protected: Liver SLCO1B3,
   e. Tissue protected: Kidney (glomeruli) ZDHHC6, ACER3, KDR, PODXL, PLA2R1, PTH2R, MAP6, NPHS2, SNCA, PTPRO, EHD1, AIF1L, CD34, ENG, AKAP12, CACNA1C, ITGA8, EHD3,
   f. Tissue protected: Kidney (tubules) SLC5A12, SLC12A1, SLC34A3, UCHL1, MGAT4B, LRRC55, SLC22A6, TMEM72, SLC3A1, TMEM174, SLC28A1, SLC13A2, SLC4A4, ENPP6, DPEP1, CUBN, AIF1L, ADCY7, CDH6, CLIC4, BSND, AQP6, ANK2, SLC7A9, ACE, AQP3, CDH16, MC4R, MARCKS, LRP2, OR10R2, NDST2,
   g. Tissue protected: Heart (cardiomyocytes) RASL10B, SLC22A15, SGCB, PLN, SGCG, PPP1R3A, DSP, SLC2A4, FAM168B, CHRNA1, CPT1B, PAM, MYZAP, MLIP, SLC16A10, OR10R2, DES,
   h. Tissue protected: Thyroid ZDHHC6, SLC5A5, TPO, LRRC55, SLC8A1, UNC13B, IYD, ANKRD13A, CDH16,
   i. Tissue protected: Pancreatic endocrine cells (Islets) ZDHHC2, SCGN, SCG3, RAB3B, UCHL1, MXRA7, PTPRN, SV2A, YRDC, TSPAN7, SLC30A8, SYP, PTPRN2, DGCR2, GNAO1, CASR, CADPS,
   j. Tissue protected: Pancreatic exocrine cells RASL10B, TMEM97, STX5, PTH2R, YRDC, SYCN, SLC4A4, DPEP1, CUZD1, KIRREL2, ADCY7, EVI2A, GPAT2, AQP12A, AQP12B, MC4R,
8. Cancer treated: stomach cancer
   a. Tissue protected: Colon UGT2A3, SLC26A2, SNX1, UGT2B17, TMEM236, UGT2B28, UGT2B15, CLCA1, HTR1A, ABCG8, GAL3ST2, SLC9A3R2,
   b. Tissue protected: Lung SLC22A15, SLC25A17, AGER, PSD4, CCR7, AOC3, GBP2, CAV1, LAMP3, SLC34A2, ICAM1,
   c. Tissue protected: Skin PERP, MTMR2, TMEM134, LYPD3, KRT1, CLCA2, DSC3, DSG1, ADGRF4, CAV1, ORAI1, FGFR3, TMEM243, SNX1, ICAM3, S100A8, FAT2, GBP2, OR10R2,
   d. Tissue protected: Liver SLCO1B3, SLC27A5, PIGU, RDH16, SORD, TFR2, CYP2D6, SLC2A2, CYP2A7, EPHX1, CYP1A2, CYP3A4, CYP2A6, FMO3, ABCB4, CYP2C8, CDH2, CYP2C19, BCL2L10, AIG1, ASGR1, ABCB11, CYP2A13, HSD11B1, CYP2C9, ABCC2, GAA,
   e. Tissue protected: Kidney (glomeruli) TMEM245, KDR, KIRREL1, PLA2R1, PTH2R, MAP6, NPHS2, TNS2, SNCA, PTPRO, SLC35B1, EHD1, CD34, ENG, CLEC2B, CACNA1C, SLC9A3R2, ITGA8, EHD3, ICAM1, DYSF,
   f. Tissue protected: Kidney (tubules) TRPM3, STRA6, SLC5A12, SLC12A1, RNF144A, SLC27A2, RDX, SLC6A19, SLC34A3, PIGU, PDZK1, KCNJ1, UCHL1, MGAT4B, LRRC55, NAT8, PALM, MFN2, UGT2B4, ATP6V1D, SLC22A6, TMEM72, XPNPEP2, SLC3A1, TMEM174, SLC28A1, SLC13A2, SYT7, SLC4A4, ENPP6, GGT1, GNAI1, CUBN, ADCY7, HTR1A, ATRAID, CLIC4, FAM210A, CDH2, BSND, BCL2L10, AQP6, ANK2, SLC7A9, FXYD2, ACE, TMEM63B, CDH16, INTS2, LRP2, OR10R2, ABCC2, LRRK2,
   g. Tissue protected: Heart (cardiomyocytes) SLC27A6, CAP2, CD36, SLC22A15, TEX2, RNF144A, SGCB, BVES, PLN, SGCG, SMPD2, CKMT2, CYP2W1, CAVIN4, SLC2A4, CHRNA1, GBP2, CDH2, CLEC2B, ATP2A2, CPT1B, COX7B, MYZAP, MLIP, OR10R2, DES,
   h. Tissue protected: Thyroid TMEM245, TPO, LRRC55, SORD, UTRN, IYD, CYP2W1, ANKRD13A, ABCG8, BCL2, CDH16, GAA,
   i. Tissue protected: Pancreatic endocrine cells (Islets) SCGN, TRPV4, SCG3, PTGDR2, RAB3B, UCHL1, SMPD2, PTPRN, SV2A, YRDC, TSPAN7, SLC30A8, SYP, DGCR2, CYP2W1, GNAO1, CASR, CD99, GBP2, LRRC24,
   j. Tissue protected: Pancreatic exocrine cells TEX2, TMEM97, PTH2R, YRDC, SYCN, SLC4A4, CUZD1, KIRREL2, ADCY7, FKBP2, EVI2A, GPAT2, GBP2, CFTR, FXYD2, SLC2A11, GAA,
9. Cancer treated: cervical cancer
   a. Tissue protected: Colon SMIM24, RNF144B, PEX10, MGLL, TMEM236, C2orf88, RAB27A, PIGZ, DPEP1, KCNMB3, FCER1G, CLCA1, CLN6, GAL3ST2, APOBR, ATP8B1, SLC9A3R2, GPA33, RPS6KC1, MUC13, MRAP,
   b. Tissue protected: Lung SLC22A15, SLC25A17, AGER, PAG1, PSD4, VTI1B, STEAP1, CCR7, CYB5A, AOC3, AP3M1, LAMP3, SLC34A2,
   c. Tissue protected: Skin GJB4, DSP, ADGRA3, GSDMA, DSG1, APOBR, FAT2, OR10R2,
   d. Tissue protected: Liver SLC38A9, SLCO1B3, SPIRE1, SLC27A5, SLC25A20, RNF144B, RDH16, TFR2, EVA1A, CYP2D6, SLC2A2, CYP2A7, CYP1A2, FXYD6, CYP3A4, CYP2A6, HHAT, ABCB4, CYP2C8, CDH2, CYP2C19, BCL2L10, ANPEP, ASGR1, ABCB11, CYP2A13, HSD11B1, CYP2C9, GK, ABCC2, DPP4,
   e. Tissue protected: Kidney (glomeruli) ACER3, KIRREL1, PLA2R1, PTH2R, MME, NPHS2, TNS2, SNCA, PTPRO, ENG, ENPEP, CACNA1C, SLC9A3R2, ITGA8, NPHS1, DYSF,
   f. Tissue protected: Kidney (tubules) TRPM3, FCAMR, SLC47A1, SMIM24, SLC5A12, SLC12A1, RNF144A, SLC27A2, SPIRE1, SLC6A19, SLC34A3, VAT1, VHL, RNF144B, SEL1L3, UCHL1, ITPR2, NAT8, MME, UGT2B4, ATP6V1D, SLC22A6, TMEM72, XPNPEP2, SLC3A1, SLC7A7, TMEM174, TMPRSS2, SLC28A1, PIGZ, SLC13A2, VTI1B, SLC4A4, ENPP6, EFHD1, GGT1, DPEP1, CUBN, HYAL2, KCNMB3, SLC48A1, DAB2, CLN6, FXYD6, CLIC4, HHAT, CDH2, BSND, BCL2L10, AQP6, AQP1, ANPEP, ENPEP, SLC7A9, FXYD2, ACE, TMEM63B, ACVRL1, TMEM37, INTS2, LFNG, LRP2, MRAP, MT-ND4, OR10R2, NDUFB9, ABCC2, DPP4, LRRK2, NDST2,
   g. Tissue protected: Heart (cardiomyocytes) GABARAPL2, RASL10B, SLC22A15, RNF144A, SGCB, BVES, PLN, PVR, SGCG, PPP1R3A, VTI1B, CKMT2, DSP, CAVIN4, CHRNA1, FXYD7, CDH2, CPT1B, ARL4C, ATP8B1, PAM, MYZAP, MRAP, OR10R2, NDUFB9, DES,
   h. Tissue protected: Thyroid ENPP1, TPO, TPCN1, MFSD11, VTI1B, IYD, IL17RA,
   i. Tissue protected: Pancreatic endocrine cells (Islets) SCGN, SCG3, VAT1, RAB3B, UCHL1, MXRA7, PTPRN, SV2A, TSPAN7, SLC30A8, SYP, PTPRN2, VTI1B, DGCR2, GNAO1, CASR, CD99, FKBP11, ATP9A, CADPS, LRRC24,
   j. Tissue protected: Pancreatic exocrine cells TMED6, RASL10B, SPIRE1, TMEM97, STX5, PTH2R, SYCN, VTI1B, SLC38A5, SLC4A4, DPEP1, CUZD1, GP2, KIRREL2, EVI2A, GPAT2, FKBP11, AQP1, ANPEP, CFTR, FXYD2, AQP12A, AQP12B, IL17RA, MRAP,
10. Cancer treated: skin cancer
   a. Tissue protected: Colon SMAGP, ATP8B2, CORO7, SELENBP1, SMIM24, TMEM94, PLXNA1, SLC25A46, PIGR, PLEKHA3, TULP3, TFPI, TJP3, RAB27B, PEX10, CD46, LYPLA1, NUCB2, TNFAIP8L3, VPS11, SIGIRR, VPS28, VPS35L, SYTL2, SLC28A3, RAB27A, PIGZ, SLC6A15, TECPR1, VPS41, CDHR5, DPEP1, KCNMB3, FCER1G, ACSL5, CLCA1, FPR2, HTR1A, CLN6, CLMN, CA12, BRI3, GAL3ST2, B3GNT8, ELOVL7, APOBR, SLC25A13, CDH17, ANKRD46, CDH26, C12orf66, SLC9A3R2, SLC2A6, GPA33, MUC13, HPS6,
   b. Tissue protected: Lung SLC22A15, AGER, PAG1, SLC6A15, VTI1B, STEAP1, CCR7, CYB5A, AOC3, AP3M1, ADGRL1, LAMP3, SLC34A2,
   c. Tissue protected: Skin DSP, APOBR, PSKH1, ELOVL1, OR10R2,
   d. Tissue protected: Liver SELENBP1, TMEM94, SLCO1B3, SLC25A43, SLC27A5, RAB20, PIGU, SIGIRR, TM4SF4, EVA1A, GHR, CYP2D6, ACSL5, SLC2A2, CYP2A7, GATM, EPHX1, CYP1A2, FXYD6, CYP3A4, CYP2A6, CYP2B6, FMO3, HHAT, CYP2C8, CDH2, CYP2C19, BCL2L10, SLC25A13, ANPEP, ASGR1, ABCB11, CYP2A13, CYP2E1, GIPC1, GK, HPS6, ABCC2, DPP4,
   e. Tissue protected: Kidney (glomeruli) PODXL, KIRREL1, PLA2R1, PTH2R, MAP6, NPHS2, TNS2, SNCA, PTPRO, SLC35B1, EHD1, CD34, ENG, AKAP12, CACNA1C, SLC9A3R2, ITGA8, EHD3, NIPAL4, NPHS1, HPS6, DYSF,
   f. Tissue protected: Kidney (tubules) TRPM3, C2CD2, ACY3, FCAMR, SLC47A1, SMIM24, SLC5A12, TMEM94, SLC12A1, RNF144A, SLC27A2, SLC6A19, SLC22A11, SLC34A3, NAGPA, PIGR, PLLP, TFPI, VHL, RAB27B, PIGU, UCHL1, MGAT4B, MITD1, KCNK1, NAT8, TMEM159, UGT2B4, ATP6V1B1, SLC22A6, TMEM72, TNFAIP8L3, SLC3A1, SLC7A7, TMEM174, SYTL2, TMPRSS2, SLC28A1, SLC13A3, PIGZ, RAB11FIP5, SLC13A2, VTI1B, SYT7, SLC4A4, PKHD1, IMMT, TRPV5, VPS41, ADCK2, CDHR5, ENPP6, GGT1, DPEP1, CUBN, HYAL2, KCNMB3, SLC48A1, ACSL5, CACNA1B, CHDH, HTR1A, CLN6, GATM, ATRAID, FXYD6, CLMN, COQ2, COX14, ELOVL1, CA12, HHAT, CDH2, SLC2A5, BSND, BCL2L10, AQP6, SLC25A13, ANK2, ANPEP, SLC7A9, CPM, FXYD2, ACE, ADGRL1, TMEM63B, ACVRL1, CDH16, INTS2, SMPD4, MOXD1, LFNG, GIPC1, MC4R, SLC2A6, LRP2, HPS6, OR10R2, SLC16A4, NDUFB9, ABCC2, DPP4, LRRK2, NDST2,
   g. Tissue protected: Heart (cardiomyocytes) VDAC3, SLC27A6, CAP2, GABARAPL2, SLC22A15, RNF144A, SGCB, BVES, RAB20, SGCG, MYORG, TMEM143, SUSD6, VTI1B, PPM1L, TECPR1, IMMT, CKMT2, CYP2W1, DSP, DOK7, CAVIN4, SLC2A4, CHRNA1, CNTFR, CDH2, CPT1B, COX7B, ARL4C, MYZAP, MSTO1, MLIP, SLC16A10, ICA1, OR10R2, NDUFB9, DES,
   h. Tissue protected: Thyroid TSPAN3, SELENBP1, ENPP1, SLC25A46, TPO, TPCN1, IFI27, HEG1, KCNQ1, MFSD11, UTRN, UNC13B, VTI1B, VPS41, IYD, CYP2W1, HLA-DQB1, COQ2, CDH16, GIPC1, MSTO1,
   i. Tissue protected: Pancreatic endocrine cells (Islets) TSPAN3, SCGN, SCG3, NBEA, PTGDR2, UCHL1, SERP1, PTPRN, MGAT4A, SHISAL2B, SV2A, YRDC, TSPAN7, SLC30A8, SYP, PTPRN2, VTI1B, DGCR2, CYP2W1, GNAO1, CASR, CD99, DIAPH1, FKBP11, ATP9A, CADPS, CADM1, LRRC24,
   j. Tissue protected: Pancreatic exocrine cells TNFSF14, C2CD2, TMEM263, TMEM97, PLEKHA3, STX5, IFI27, MITD1, PTH2R, VPS11, YRDC, VAMP1, SYCN, VTI1B, SLC4A4, SRPRB, DPEP1, CUZD1, GP2, KIFC3, KIRREL2, FKBP2, EVI2A, GATM, AQP8, DIAPH1, GPAT2, FKBP11, CA12, ELOVL7, ANPEP, CFTR, FXYD2, ADGRL1, AQP12A, AQP12B, MOXD1, MC4R, HPS6, PDLIM4,
11. Cancer treated: endometrial cancer
   a. Tissue protected: Colon UGT2A3, TMEM60, SMIM24, TMEM45A, UGT2B17, TMEM106C, VPS35L, UGT2B28, TMEM253, SYTL2, UGT2B 15, PIGZ, SLC39A14, SLC6A15, TSNARE1, CDHR5, DPEP1, EMC9, CLCA1, FPR2, CLN6, BRI3, GAL3ST2, B3GNT8, FAM162A, CDH17, C12orf66, HSD3B7, PYCARD, GPA33, MUC13,
   b. Tissue protected: Lung SLC22A15, SLC25A17, AGER, SDR16C5, PSD4, SLC6A15, STEAP1, CCR7, CYB5A, GPCS, AOC3, CAV1, CAV2, LAMP3,
   c. Tissue protected: Skin PERP, RAC1, PPP1R16B, MTMR2, SDR16C5, LYPD3, KRT1, LY6D, TSNARE1, GJB4, DSP, DSC3, EPHA10, DSG1, CAV1, CAV2, ICAM3, S100A8, FAT2, PYCARD, OR10R2,
   d. Tissue protected: Liver SLCO1B3, SLC27A5, RDH16, PLPPR2, TFR2, TSNARE1, EVA1A, CYP2D6, SLC2A2, CYP2A7, CYP1A2, CYP3A4, CYP2A6, CYP2B6, FMO3, ABCB4, CYP2C8, CYP2C19, BCL2L10, ASGR1, ABCB11, CYP2A13, DCXR, CYP2C9, ABCC2,
   e. Tissue protected: Kidney (glomeruli) VIPR2, KDR, PLA2R1, PTH2R, MME, NPHS2, TNS2, SNCA, PTPRO, EHD1, CD34, ENG, CLEC2B, ENPEP, ITGA8, EHD3, NPHS1,
   f. Tissue protected: Kidney (tubules) TRPM3, SMIM24, SLC5A12, SLC12A1, SLC6A19, SLC22A11, SLC34A3, SEL1L3, MGAT4B, PRKN, NAT8, MME, UGT2B4, SLC22A6, TMEM72, XPNPEP2, SLC3A1, SLC7A7, TMEM106C, TMEM174, SYTL2, TMPRSS2, SLC28A1, SLC13A3, PIGZ, SLC13A2, SLC4A4, TSNARE1, CDHR5, ENPP6, EFHD1, DPEP1, CUBN, HYAL2, DAB2, FAM151A, CLN6, ATRAID, FAM210A, SLC7A8, BSND, BCL2L10, AQP6, AQP1, ENPEP, SLC7A9, FXYD2, ACE, TMEM63B, DCXR, HSD3B7, LRP2, OR10R2, ABCC2, LRRK2,
   g. Tissue protected: Heart (cardiomyocytes) CD36, RASL10B, SLC22A15, BVES, PARVB, PLN, SGCG, MYORG, TMEM106C, TMEM143, TOMM40L, PRRG2, TMX2, CKMT2, CYP2W1, DSP, CAVIN4, FAM168B, CHRNA1, CLEC2B, CAV2, MLIP, OR10R2, DES,
   h. Tissue protected: Thyroid ENPP1, TPO, SLC8A1, UTRN, UNC13B, RAB43, IYD, EMC9, CYP2W1, MAP1LC3B,
   i. Tissue protected: Pancreatic endocrine cells (Islets) SCGN, SCG3, PTGDR2, SERP1, PTPRN, YRDC, TSPAN7, SLC30A8, SYP, PTPRN2, DGCR2, CYP2W1, GNAO1, CASR, CADPS,
   j. Tissue protected: Pancreatic exocrine cells TMED6, RASL10B, PPP1R16B, PTH2R, YRDC, SYCN, SLC38A5, SLC4A4, DPEP1, EMC9, CUZD1, KIRREL2, AQP8, GPAT2, AQP1, FAM162A, FXYD2, AQP12A, AQP12B,
12. Cancer treated: breast cancer
   a. Tissue protected: Colon ZDHHC2, UGT2A3, TMEM60, SLC26A2, TMEM45A, PEX10, PLOD1, UGT2B17, UGT2B28, TMEM253, UGT2B15, PIGZ, SLC39A14, SLC6A15, SDCBP2, CDHR5, DPEP1, ACSL5, CLCA1, GBA2, FPR2, HTR1A, CEACAM5, BRI3, GAL3ST2, B3GNT8, CDH17, EPB41L2, CDH26, GPA33, MUC13,
   b. Tissue protected: Lung SLC22A15, SLC25A17, AGER, SLC6A15, STEAP1, CCR7, CAV1, CAV2, EPB41L2, LAMP3,
   c. Tissue protected: Skin PERP, TMEM134, KRT1, SRC, LY6D, FGFR2, DSP, DSC3, EPHA10, DSG1, CAV1, CAV2, ORAI1, TMEM243, FAT2, FGFBP1, OR10R2,
   d. Tissue protected: Liver SLCO1B3, SLC25A43, SLC27A5, SLC25A20, TFR2, CRK, EVA1A, CYP2D6, ACSL5, SLC2A2, CYP2A7, CYP1A2, CYP3A4, CYP2B6, FMO3, CYP2C8, CDH2, CYP2C19, BCL2L10, AIG1, ASGR1, ABCB11, DCXR, HSD11B1, CYP2C9, ABCC2, DPP4,
   e. Tissue protected: Kidney (glomeruli) ZDHHC6, ACER3, VIPR2, KDR, PODXL, KIRREL1, PLPP1, PLA2R1, PTH2R, MAP6, MME, NPHS2, SNCA, RYK, PTPRO, SLC35B1, EHD1, CD34, ENG, ENPEP, ITGA8, EHD3, DYSF,
   f. Tissue protected: Kidney (tubules) TRPM3, C2CD2, FCAMR, SLC5A12, SLC12A1, SLC6A19, SLC34A3, VPS26B, VHL, KCNJ1, SEL1L3, UCHL1, MGAT4B, LRRC55, NAT8, MME, UGT2B4, SLC22A6, TMEM72, SLC7A7, UGT1A6, TMEM174, TMPRSS2, SLC28A1, SLC13A3, PIGZ, SLC13A2, SLC4A4, CRK, CDHR5, ENPP6, DPEP1, CUBN, HYAL2, ACSL5, CDH6, FAM151A, HTR1A, CLIC4, CDH2, SLC7A8, BSND, BCL2L10, AQP6, AQP1, ENPEP, FXYD2, ACE, DCXR, CDH16, OR10R2, NDUFB9, ABCC2, DPP4,
   g. Tissue protected: Heart (cardiomyocytes) SLC22A15, SGCB, NDUFS8, PLN, PVR, SGCG, TMEM143, CKMT2, DSP, FAM168B, CHRNA1, CDH2, CAV2, CPT1B, COX7B, MLIP, OR10R2, NDUFB9, DES,
   h. Tissue protected: Thyroid ZDHHC6, SLC5A5, LPCAT2, RETSAT, TPO, IFI27, LRRC55, IYD, GBA2, CDH16,
   i. Tissue protected: Pancreatic endocrine cells (Islets) ZDHHC2, SCG3, PTGDR2, RAB3B, UCHL1, PTPRN, SHISAL2B, SV2A, TSPAN7, SLC30A8, SYP, DGCR2, GNAO1, CASR, CD99, CADPS,
   j. Tissue protected: Pancreatic exocrine cells C2CD2, TMEM97, IFI27, PTH2R, SYCN, SLC4A4, DPEP1, CUZD1, KIFC3, KIRREL2, GPAT2, AQP1, CFTR, FXYD2, AQP12A, AQP12B,

Based on the description of biophysical and expression criteria for PD-1 binding moiety and surface antigen-binding moiety pairs, the following are representative, nonlimiting, and additional examples of additional LoSTIM constructs are provided in Table 4B below.

TABLE 4B

Anti-EpCAM clone 9C4
Heavy Chain:
QIQLVQSGPELKKPGETVKISCKASGYTFTNYLLNWVKQAPGKGLKWMGWINTYTGEPTYTDDFKGRFAFSLATSASTAYLQI NNLKNEDTATYFCVRLAGTKDYWGQGTTLTVSS

Light Chain:
DlVMTQAAFSNPVTLGTSASISCRSSKSLLHSNGiTYLYWYLQKPGQSPQLLIYQMSNLASGVPDRFSSSGSGTDFTLRISRVE AEDVGVYYCAQNLELPRTFGGGTKLEIK

Anti-EpCAM clone MOC31
Heavy Chain:
QVKLQQSGPELKKPGETVKISCKASGYTFTNYGMNWVKQAPGKGLKWMGWINTYTGESTYADDFKGRFAFSLETSASAAYL QINNLKNEDTATYFCARFAIKGDYWGQGTTVTVSS

Light Chain:
DIVLTQSPFSNPVTLGTSASISCRSTKSLLHSNGITYLYWYLQKPGQSPQLLIYQMSNLASGVPDRFSSSGSGTDFTLRISRVE AEDVGVYYCAQNLEIPRTFGGGTKLEIK

Anti-EpCAM adecatumumab
Heavy Chain:
EVQLLESGGGVVQPGRSLRLSCAASGFTFSSYGMHWVRQAPGKGLEWVAVISYDGSNKYYADSVKGRFTISRDNSKNTLYL QMNSLRAEDTAVYYCAKDMGWGSGWRPYYYYGMDVWGQGTTVTVSS Light Chain: ELQMTQSPSSLSASVGDRVTITCRTSQSISSYLNWYQQKPGQPPKLLIYWASTRESGVPDRFSGSGSGTDFTLTISSLQPEDS ATYYCQQSYDIPYTFGQGTKLEIK

Anti-HLA-A2 clone BB7.2
Heavy Chain:
QVQLQQSGPELVKPGASVKMSCKASGYTFTSYHIQWVKQRPGQGLEWIGWIYPGDGSTQYNEKFKGKTTLTADKSSSTAYM LLSSLTSEDSAIYFCAREGTYYAMDYWGQGTSVTVSS Light Chain: DVLMTQTPLSLPVSLGDQVSISCRSSQSIVHSNGNTYLEWYLQKPGQSPKLLIYKVSNRFSGVPDRFSGSGSGTDFTLKISRV EAEDLGVYYCFQGSHVPRTFGGGTKLEIK

Anti-MHC II (pan-allele) clone 9.49
Heavy Chain:
EVQLQESGPSLVRPSQTLSLTCSVTGDSITSSYWNWIRKFPGNKLEYMGYITYSGNTYYTPSLKSRISITRDTSKNQYFLQLNS VTTEDTATYYCSRDYGRGDYAMVYWGQGTSVTVSS Light Chain: DVVLTQSPATLSVTPGDSVSLSCRASQSIINNLHWYHQKSHESPRLLIKYASQSISGIPSRFSGSGSGTDFTLSINSVETEDFGV YFCQQSGSWPYTFGGGSNLEIK

Anti-PD-1 (pembrolizumab)
Heavy Chain:
QVQLVQSGVEVKKPGASVKVSCKASGYTFTNYYMYWVRQAPGQGLEWMGGINPSNGGTNFNEKFKNRVTLTTDSSTTTAY MELKSLQFDDTAVYYCARRDYRFDMGFDYWGQGTTVTVSS

Light Chain:
EIVLTQSPATLSLSPGERATLSCRASKGVSTSGYSYLHWYQQKPGQAPRLLIYLASYLESGVPARFSGSGSGTDFTLTISSLEP EDFAVYYCQHSRDLPLTFGGGTKVEIK

Anti-PD-1 (nivolumab)
Heavy Chain:
QVQLVESGGGVVQPGRSLRLDCKASGITFSNSGMHWVRQAPGKGLEWVAVIWYDGSKRYYADSVKGRFTISRDNSKNTLFL QMNSLRAEDTAVYYCATNDDYWGQGTLVTVSS

Light Chain:
EIVLTQSPATLSLSPGERATLSCRASQSVSSYLAWYQQKPGQAPRLLIYDASNRATGIPARFSGSGSGTDFTLTISSLEPEDFA VYYCQQSSNWPRTFGQGTKVEIK

Kappa liqht chain constant region
RTVAAPSVFIFPPSDEQLKSGTASVVCLLNNFYPREAKVQWKVDNALQSGNSQESVTEQDSKDSTYSLSSTLTLSKADYEKH KVYACEVTHQGLSSPVTKSFNRGEC

IqG4-SPLE
ASTKGPSVFPLAPCSRSTSESTAALGCLVKDYFPEPVTVSWNSGALTSGVHTFPAVLQSSGLYSLSSVVTVPSSSLGTKTYTC NVDHKPSNTKVDKRVESKYGPPCPSCPAPEFLGGPSVFLFPPKPKDTLMISRTPEVTCVVVDVSQEDPEVQFNWYVDGVEV HNAKTKPREEQFNSTYRVVSVLTVLHQDWLNGKEYKCKVSNKGLPSSIEKTISKAKGQPREPQVYTLPPSQEEMTKNQVSLT CLVKGFYPSDIAVEWESNGQPENNYKTTPPVLDSDGSFFLYSRLTVDKSRWQEGNVFSCSVMHEALHNHYTQKSLSLSLGK

IgG1-LALA-KnoblT366V1O
ASTKGPSVFPLAPSSKSTSGGTAALGCLVKDYFPEPVTVSWNSGALTSGVHTFPAVLQSSGLYSLSSVVTVPSSSLGTQTYIC NVNHKPSNTKVDKKVEPKSCDKTHTCPPCPAPEAAGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTISKAKGQPREPQVYTLPPSRDELTKNQVSL WCLVKGFYPSDIAVEWESNGQPENNYKTTPPVLDSDGSFFLYSKLTVDKSRWQQGNVFSCSVMHEALHNHYTQKSLSLSPG K

Hole(T366S,L368AandY407V):
ASTKGPSVFPLAPSSKSTSGGTAALGCLVKDYFPEPVTVSWNSGALTSGVHTFPAVLQSSGLYSLSSVVTVPSSSLGTQTYIC NVNHKPSNTKVDKKVEPKSCDKTHTCPPCPAPEAAGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTISKAKGQPREPQVYTLPPSRDELTKNQVSL SCAVKGFYPSDIAVEWESNGQPENNYKTTPPVLDSDGSFFLVSKLTVDKSRWQQGNVFSCSVMHEALHNHYTQKSLSLSPG K

G4S linker to connect VH and VL regions to construct scFv
GGGGSGGGGSGGGGS

G4AG4 linker to separate domains
GGGGAGGGG

### > Seq 32: 9C4-Pembrolizumab (EpCAM-PD-1)IgG4-SPLE C-Terminal scFv Bivalent Bispecific sAg-PD-1 scFv Orientation 1

> Heavy Chain:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPC SRSTSESTAALGCLVKDY
FPEPVTVSWNSGALTSGVHTFPAVLQSSGLYSLSSVVTVPSSSLGTK TY
TCNVDHKPSNTKVDKRVESKYGPPCPSCPAPEFLGGPSVFLFPPKPKDTL
MISRTPEVTCVVV DVSQEDPEVQFNWYVDGVEVHNAKTKPREEQFNSTY
RVVSVLTVLHQDWLNGKEYKCKVSNKGLP SSIEKTISKAKGQPREPQVY
TLPPSQEEMTKNQVSLTCLVKGFYPSDIAVEWESNGQPENNYKTT PPVL
DSDGSFFLYSRLTVDKSRWQEGNVFSCSVMHEALHNHYTQKSLSLSLGKG
GGGAGGGGQVQ LVQSGVEVKKPGASVKVSCKASGYTFTNYYMYWVRQAP
GQGLEWMGGINPSNGGTNFNEKFKNRV TLTTDSSTTTAYMELKSLQFDD
TAVYYCARRDYRFDMGFDYWGQGTTVTVSSGGGGSGGGGSGGG GSEIVL
TQSPATLSLSPGERATLSCRASKGVSTSGYSYLHWYQQKPGQAPRLLIYL
ASYLESGVP ARFSGSGSGTDFTLTISSLEPEDFAVYYCQHSRDLPLTFG
GGTKVEIK

### > Seq 33: 9C4-Pembrolizumab (EpCAM-PD-1)IgG4-SPLE C-terminal scFv Bivalent Bispecific sAg-PD-1 scFv Orientation 1

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 34: 9C4-Pembrolizumab IgG4-SPLE C-Terminal scFv Bivalent Bispecific sAg-PD-1 scFv Orientation 2

> Heavy Chain:

QIQLVQSGPELKKPGETVKISCKASGYTFTNYLLNWVKQAPGKGLKWMG
WINTYTGEPTYTDDFK GRFAFSLATSASTAYLQINNLKNEDTATYFCVR
LAGTKDYWGQGTTLTVSSASTKGPSVFPLAPC SRSTSESTAALGCLVKD
YFPEPVTVSWNSGALTSGVHTFPAVLQSSGLYSLSSVVTVPSSSLGTK T
YTCNVDHKPSNTKVDKRVESKYGPPCPSCPAPEFLGGPSVFLFPPKPKDT
LMISRTPEVTCVVV DVSQEDPEVQFNWYVDGVEVHNAKTKPREEQFNST
YRVVSVLTVLHQDWLNGKEYKCKVSNKGLP SSIEKTISKAKGQPREPQV
YTLPPSQEEMTKNQVSLTCLVKGFYPSDIAVEWESNGQPENNYKTT PPV
LDSDGSFFLYSRLTVDKSRWQEGNVFSCSVMHEALHNHYTQKSLSLSLGK
GGGGAGGGGEIV LTQSPATLSLSPGERATLSCRASKGVSTSGYSYLHWY
QQKPGQAPRLLIYLASYLESGVPARFSG SGSGTDFTLTISSLEPEDFAV
YYCQHSRDLPLTFGGGTKVEIKGGGGSGGGGSGGGGSQVQLVQS GVEVK
KPGASVKVSCKASGYTFTNYYMYWVRQAPGQGLEWMGGINPSNGGTNFNE
KFKNRVTLTT DSSTTTAYMELKSLQFDDTAVYYCARRDYRFDMGFDYWG
QGTTVTVSS

### > Seq 35: 9C4-Pembrolizumab (EpCAM-PD-1)IgG4-SPLE C-terminal scFv Bivalent Bispecific sAg-PD-1 scFv Orientation 2

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 36: 9C4-Pembrolizumab IgG4-SPLE C-Terminal scFv Bivalent Bispecific PD-1-sAg scFv Orientation 1

> Heavy Chain:

QVQLVQSGVEVKKPGASVKVSCKASGYTFTNYYMYWVRQAPGQGLEWMG
GINPSNGGTNFNEKFK NRVTLTTDSSTTTAYMELKSLQFDDTAVYYCAR
RDYRFDMGFDYWGQGTTVTVSSASTKGPSVFP LAPCSRSTSESTAALGC
LVKDYFPEPVTVSWNSGALTSGVHTFPAVLQSSGLYSLSSVVTVPSSS L
GTKTYTCNVDHKPSNTKVDKRVESKYGPPCPSCPAPEFLGGPSVFLFPPK
PKDTLMISRTPEVT CVVVDVSQEDPEVQFNWYVDGVEVHNAKTKPREEQ
FNSTYRVVSVLTVLHQDWLNGKEYKCKVSN KGLPSSIEKTISKAKGQPR
EPQVYTLPPSQEEMTKNQVSLTCLVKGFYPSDIAVEWESNGQPENN YKT
TPPVLDSDGSFFLYSRLTVDKSRWQEGNVFSCSVMHEALHNHYTQKSLSL
SLGKGGGGAGGG GQIQLVQSGPELKKPGETVKISCKASGYTFTNYLLNW
VKQAPGKGLKWMGWINTYTGEPTYTDDF KGRFAFSLATSASTAYLQINN
LKNEDTATYFCVRLAGTKDYWGQGTTLTVSSGGGGSGGGGSGGG GSDIV
MTQAAFSNPVTLGTSASISCRSSKSLLHSNGITYLYWYLQKPGQSPQLLI
YQMSNLASGV PDRFSSSGSGTDFTLRISRVEAEDVGVYYCAQNLELPRT
FGGGTKLEIK

### > Seq 37: 9C4-Pembrolizumab (EpCAM-PD-1)IgG4-SPLE C-Terminal scFv Bivalent Bispecific PD-1-sAg scFv Orientation 1

> Light Chain:

EIVLTQSPATLSLSPGERATLSCRASKGVSTSGYSYLHWYQQKPGQAPR
LLIYLASYLESGVPAR FSGSGSGTDFTLTISSLEPEDFAVYYCQHSRDL
PLTFGGGTKVEIKRTVAAPSVFIFPPSDEQLK SGTASVVCLLNNFYPRE
AKVQWKVDNALQSGNSQESVTEQDSKDSTYSLSSTLTLSKADYEKHKV Y
ACEVTHQGLSSPVTKSFNRGEC

### > Seq 38: 9C4-Pembrolizumab IgG4-SPLE C-Terminal scFv Bivalent Bispecific PD1-sAg scFv Orientation 2

> Heavy Chain:

QVQLVQSGVEVKKPGASVKVSCKASGYTFTNYYMYWVRQAPGQGLEWMG
GINPSNGGTNFNEKFK NRVTLTTDSSTTTAYMELKSLQFDDTAVYYCAR
RDYRFDMGFDYWGQGTTVTVSSASTKGPSVFP LAPCSRSTSESTAALGC
LVKDYFPEPVTVSWNSGALTSGVHTFPAVLQSSGLYSLSSVVTVPSSS L
GTKTYTCNVDHKPSNTKVDKRVESKYGPPCPSCPAPEFLGGPSVFLFPPK
PKDTLMISRTPEVT CVVVDVSQEDPEVQFNWYVDGVEVHNAKTKPREEQ
FNSTYRVVSVLTVLHQDWLNGKEYKCKVSN KGLPSSIEKTISKAKGQPR
EPQVYTLPPSQEEMTKNQVSLTCLVKGFYPSDIAVEWESNGQPENN YKT
TPPVLDSDGSFFLYSRLTVDKSRWQEGNVFSCSVMHEALHNHYTQKSLSL
SLGKGGGGAGGG GDIVMTQAAFSNPVTLGTSASISCRSSKSLLHSNGIT
YLYWYLQKPGQSPQLLIYQMSNLASGVP DRFSSSGSGTDFTLRISRVEA
EDVGVYYCAQNLELPRTFGGGTKLEIKGGGGSGGGGSGGGGSQI QLVQS
GPELKKPGETVKISCKASGYTFTNYLLNWVKQAPGKGLKWMGWINTYTGE
PTYTDDFKGR FAFSLATSASTAYLQINNLKNEDTATYFCVRLAGTKDYW
GQGTTLTVSS

### > Seq 39: 9C4-Pembrolizumab (EpCAM-PD-1)IgG4-SPLE C-Terminal scFv Bivalent Bispecific PD1-sAg scFv Orientation 2

> Light Chain:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 40: 9C4-Pembrolizumab IgG1-KIH-LALA C-Terminal scFv PD1 Monovalent, sAg Bivalent Bispecific 1

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG QVQLVQSGVEVKKPGASVKVSCKASGYTFTNYYMYWVR
QAPGQGLEWMGGINPSNGGTNFNEKFK NRVTLTTDSSTTTAYMELKSLQ
FDDTAVYYCARRDYRFDMGFDYWGQGTTVTVSSGGGGSGGGGS GGGGSE
IVLTQSPATLSLSPGERATLSCRASKGVSTSGYSYLHWYQQKPGQAPRLL
IYLASYLES GVPARFSGSGSGTDFTLTISSLEPEDFAVYYCQHSRDLPL
TFGGGTKVEIK

### > Seq 41: 9C4-Pembrolizumab IgG1-KIH-LALA C-Terminal scFv PD1 Monovalent, sAg Bivalent Bispecific 1

> Heavy Chain 2:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 42: 9C4-Pembrolizumab (EpCAM-PD-1)IgG1-KIH-LALA C-Terminal scFv PD1 Monovalent, sAg Bivalent Bispecific 1

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 43: 9C4-Pembrolizumab IgG1-KIH-LALA C-Terminal scFv PD1 Monovalent, sAg Bivalent Bispecific 2

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG EIVLTQSPATLSLSPGERATLSCRASKGVSTSGYSYLH
WYQQKPGQAPRLLIYLASYLESGVPAR FSGSGSGTDFTLTISSLEPEDF
AVYYCQHSRDLPLTFGGGTKVEIKGGGGSGGGGSGGGGSQVQL VQSGVE
VKKPGASVKVSCKASGYTFTNYYMYWVRQAPGQGLEWMGGINPSNGGTNF
NEKFKNRVT LTTDSSTTTAYMELKSLQFDDTAVYYCARRDYRFDMGFDY
WGQGTTVTVSS

### > Seq 43: 9C4-Pembrolizumab IgG1-KIH-LALA C-Terminal scFv PD1 Monovalent, sAg Bivalent Bispecific 2

> Heavy Chain 2:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 44: 9C4-Pembrolizumab (EpCAM-PD-1)IgG1-KIH-LALA C-Terminal scFv PD1 Monovalent, sAg Bivalent Bispecific 2

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 45: 9C4-Pembrolizumab IgG1-KIH-LALA C-Terminal scFv PD1 Monovalent, sAg Bivalent Bispecific 3

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG QVQLVQSGVEVKKPGASVKVSCKASGYTFTNYYMYWVR
QAPGQGLEWMGGINPSNGGTNFNEKFK NRVTLTTDSSTTTAYMELKSLQ
FDDTAVYYCARRDYRFDMGFDYWGQGTTVTVSSGGGGSGGGGS GGGGSE
IVLTQSPATLSLSPGERATLSCRASKGVSTSGYSYLHWYQQKPGQAPRLL
IYLASYLES GVPARFSGSGSGTDFTLTISSLEPEDFAVYYCQHSRDLPL
TFGGGTKVEIK

### > Seq 46: 9C4-Pembrolizumab IgG1-KIH-LALA C-Terminal scFv PD1 Monovalent, sAg Bivalent Bispecific 3

> Heavy Chain 2:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 47: 9C4-Pembrolizumab (EpCAM-PD-1)IgG1-KIH-LALA C-Terminal scFv PD1 Monovalent, sAg Bivalent Bispecific 3

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 48: 9C4-Pembrolizumab IgG1-KIH-LALA C-Terminal scFv PD1 Monovalent, sAg Bivalent Bispecific 4

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG EIVLTQSPATLSLSPGERATLSCRASKGVSTSGYSYLH
WYQQKPGQAPRLLIYLASYLESGVPAR FSGSGSGTDFTLTISSLEPEDF
AVYYCQHSRDLPLTFGGGTKVEIKGGGGSGGGGSGGGGSQVQL VQSGVE
VKKPGASVKVSCKASGYTFTNYYMYWVRQAPGQGLEWMGGINPSNGGTNF
NEKFKNRVT LTTDSSTTTAYMELKSLQFDDTAVYYCARRDYRFDMGFDY
WGQGTTVTVSS

### > Seq 49: 9C4-Pembrolizumab (EpCAM-PD-1)IgG1-KIH-LALA C-Terminal scFv PD1 Monovalent, sAg Bivalent Bispecific 4

> Heavy Chain 2:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 50: 9C4-Pembrolizumab (EpCAM-PD-1)IgG1-KIH-LALA C-Terminal scFv PD1 Monovalent, sAg Bivalent Bispecific 4

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 51: 9C4-Pembrolizumab IgG1-KIH-LALA Monovalent Bispecific 1

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 52: 9C4-Pembrolizumab (EpCAM-PD-1)IgG1-KIH-LALA Monovalent Bispecific 1

> Heavy Chain 2:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPSSKSTSGGTAALGCL
VKDYFPEPVTVSWNSGALTSGVHTFPAVLQSSGLYSLSSVVTVPSSS LG
TQTYICNVNHKPSNTKVDKKVEPKSCDKTHTCPPCPAPEAAGGPSVFLFP
PKPKDTLMISRTP EVTCVVVDVSHEDPEVKFNWYVDGVEVHNAKTKPRE
EQYNSTYRVVSVLTVLHQDWLNGKEYKCK VSNKALPAPIEKTISKAKGQ
PREPQVYTLPPSRDELTKNQVSLWCLVKGFYPSDIAVEWESNGQP ENNY
KTTPPVLDSDGSFFLYSKLTVDKSRWQQGNVFSCSVMHEALHNHYTQKSL
SLSPGK

### > Seq 53: 9C4-Pembrolizumab (EpCAM-PD-1)IgG1-KIH-LALA Monovalent Bispecific 1

> Light Chain 1:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 54: 9C4-Pembrolizumab (EpCAM-PD-1)IgG1-KIH-LALA Monovalent Bispecific 1

> Light Chain 2:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 55: 9C4-Pembrolizumab IgG1-KIH-LALA Monovalent Bispecific 2

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 56: 9C4-Pembrolizumab (EpCAM-PD-1)IgG1-KIH-LALA Monovalent Bispecific 2

> Heavy Chain 2:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPSSKSTSGGTAALGCL
VKDYFPEPVTVSWNSGALTSGVHTFPAVLQSSGLYSLSSVVTVPSSS LG
TQTYICNVNHKPSNTKVDKKVEPKSCDKTHTCPPCPAPEAAGGPSVFLFP
PKPKDTLMISRTP EVTCVVVDVSHEDPEVKFNWYVDGVEVHNAKTKPRE
EQYNSTYRVVSVLTVLHQDWLNGKEYKCK VSNKALPAPIEKTISKAKGQ
PREPQVYTLPPSRDELTKNQVSLWCLVKGFYPSDIAVEWESNGQP ENNY
KTTPPVLDSDGSFFLYSKLTVDKSRWQQGNVFSCSVMHEALHNHYTQKSL
SLSPGK

### > Seq 57: 9C4-Pembrolizumab (EpCAM-PD-1)IgG1-KIH-LALA Monovalent Bispecific 2

> Light Chain 1:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 58: 9C4-Pembrolizumab (EpCAM-PD-1)IgG1-KIH-LALA Monovalent Bispecific 2

> Light Chain 2:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 59: 9C4-Nivolumab (EpCAM-PD-1)IgG4-SPLE C-Terminal scFv Bivalent Bispecific sAg-PD-1 scFv Orientation 1

> Heavy Chain:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPC SRSTSESTAALGCLVKDY
FPEPVTVSWNSGALTSGVHTFPAVLQSSGLYSLSSVVTVPSSSLGTK TY
TCNVDHKPSNTKVDKRVESKYGPPCPSCPAPEFLGGPSVFLFPPKPKDTL
MISRTPEVTCVVV DVSQEDPEVQFNWYVDGVEVHNAKTKPREEQFNSTY
RVVSVLTVLHQDWLNGKEYKCKVSNKGLP SSIEKTISKAKGQPREPQVY
TLPPSQEEMTKNQVSLTCLVKGFYPSDIAVEWESNGQPENNYKTT PPVL
DSDGSFFLYSRLTVDKSRWQEGNVFSCSVMHEALHNHYTQKSLSLSLGKG
GGGAGGGGQVQ LVESGGGVVQPGRSLRLDCKASGITFSNSGMHWVRQAP
GKGLEWVAVIWYDGSKRYYADSVKGRF TISRDNSKNTLFLQMNSLRAED
TAVYYCATNDDYWGQGTLVTVSSGGGGSGGGGSGGGGSEIVLT QSPATL
SLSPGERATLSCRASQSVSSYLAWYQQKPGQAPRLLIYDASNRATGIPAR
FSGSGSGTD FTLTISSLEPEDFAVYYCQQSSNWPRTFGQGTKVEIK

### > Seq 60: 9C4-Nivolumab (EpCAM-PD-1)IgG4-SPLE C-Terminal scFv Bivalent Bispecific sAg-PD-1 scFv Orientation 1

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 61: 9C4-Nivolumab IgG4-SPLE C-Terminal scFv Bivalent Bispecific sAg-PD-1 scFv Orientation 2

> Heavy Chain:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPC SRSTSESTAALGCLVKDY
FPEPVTVSWNSGALTSGVHTFPAVLQSSGLYSLSSVVTVPSSSLGTK TY
TCNVDHKPSNTKVDKRVESKYGPPCPSCPAPEFLGGPSVFLFPPKPKDTL
MISRTPEVTCVVV DVSQEDPEVQFNWYVDGVEVHNAKTKPREEQFNSTY
RVVSVLTVLHQDWLNGKEYKCKVSNKGLP SSIEKTISKAKGQPREPQVY
TLPPSQEEMTKNQVSLTCLVKGFYPSDIAVEWESNGQPENNYKTT PPVL
DSDGSFFLYSRLTVDKSRWQEGNVFSCSVMHEALHNHYTQKSLSLSLGKG
GGGAGGGGEIV LTQSPATLSLSPGERATLSCRASQSVSSYLAWYQQKPG
QAPRLLIYDASNRATGIPARFSGSGSG TDFTLTISSLEPEDFAVYYCQQ
SSNWPRTFGQGTKVEIKGGGGSGGGGSGGGGSQVQLVESGGGV VQPGRS
LRLDCKASGITFSNSGMHWVRQAPGKGLEWVAVIWYDGSKRYYADSVKGR
FTISRDNSK NTLFLQMNSLRAEDTAVYYCATNDDYWGQGTLVTVSS

### > Seq 62: 9C4-Nivolumab (EpCAM-PD-1)IgG4-SPLE C-Terminal scFv Bivalent Bispecific sAg-PD-1 scFv Orientation 2

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 63: 9C4-Nivolumab IgG4-SPLE C-Terminal scFv Bivalent Bispecific PD-1-sAg scFv Orientation 1

> Heavy Chain:

QVQLVESGGGVVQPGRSLRLDCKASGITFSNSGMHWVRQAPGKGLEWVA
VIWYDGSKRYYADSVK GRFTISRDNSKNTLFLQMNSLRAEDTAVYYCAT
NDDYWGQGTLVTVSSASTKGPSVFPLAPCSRS TSESTAALGCLVKDYFP
EPVTVSWNSGALTSGVHTFPAVLQSSGLYSLSSVVTVPSSSLGTKTYT C
NVDHKPSNTKVDKRVESKYGPPCPSCPAPEFLGGPSVFLFPPKPKDTLMI
SRTPEVTCVVVDVS QEDPEVQFNWYVDGVEVHNAKTKPREEQFNSTYRV
VSVLTVLHQDWLNGKEYKCKVSNKGLPSSI EKTISKAKGQPREPQVYTL
PPSQEEMTKNQVSLTCLVKGFYPSDIAVEWESNGQPENNYKTTPPV LDS
DGSFFLYSRLTVDKSRWQEGNVFSCSVMHEALHNHYTQKSLSLSLGKGGG
GAGGGGQIQLVQ SGPELKKPGETVKISCKASGYTFTNYLLNWVKQAPGK
GLKWMGWINTYTGEPTYTDDFKGRFAFS LATSASTAYLQINNLKNEDTA
TYFCVRLAGTKDYWGQGTTLTVSSGGGGSGGGGSGGGGSDIVMT QAAFS
NPVTLGTSASISCRSSKSLLHSNGITYLYWYLQKPGQSPQLLIYQMSNLA
SGVPDRFSSS GSGTDFTLRISRVEAEDVGVYYCAQNLELPRTFGGGTKL
EIK

### > Seq 64: 9C4-nivolumab (EpCAM-PD-1)IgG4-SPLE C-Terminal scFv Bivalent Bispecific PD-1-sAg scFv Orientation 1

> Light Chain:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 65: 9C4-Nivolumab IgG4-SPLE C-Terminal scFv Bivalent Bispecific PD1-sAg scFv Orientation 2

> Heavy Chain:

QVQLVESGGGVVQPGRSLRLDCKASGITFSNSGMHWVRQAPGKGLEWVA
VIWYDGSKRYYADSVK GRFTISRDNSKNTLFLQMNSLRAEDTAVYYCAT
NDDYWGQGTLVTVSSASTKGPSVFPLAPCSRS TSESTAALGCLVKDYFP
EPVTVSWNSGALTSGVHTFPAVLQSSGLYSLSSVVTVPSSSLGTKTYT C
NVDHKPSNTKVDKRVESKYGPPCPSCPAPEFLGGPSVFLFPPKPKDTLMI
SRTPEVTCVVVDVS QEDPEVQFNWYVDGVEVHNAKTKPREEQFNSTYRV
VSVLTVLHQDWLNGKEYKCKVSNKGLPSSI EKTISKAKGQPREPQVYTL
PPSQEEMTKNQVSLTCLVKGFYPSDIAVEWESNGQPENNYKTTPPV LDS
DGSFFLYSRLTVDKSRWQEGNVFSCSVMHEALHNHYTQKSLSLSLGKGGG
GAGGGGDIVMTQ AAFSNPVTLGTSASISCRSSKSLLHSNGITYLYWYLQ
KPGQSPQLLIYQMSNLASGVPDRFSSSG SGTDFTLRISRVEAEDVGVYY
CAQNLELPRTFGGGTKLEIKGGGGSGGGGSGGGGSQIQLVQSGP ELKKP
GETVKISCKASGYTFTNYLLNWVKQAPGKGLKWMGWINTYTGEPTYTDDF
KGRFAFSLAT SASTAYLQINNLKNEDTATYFCVRLAGTKDYWGQGTTLT
VSS

### > Seq 66: 9C4-Nivolumab (EpCAM-PD-1)IgG4-SPLE C-Terminal scFv Bivalent Bispecific PD1-sAg scFv Orientation 2

> Light Chain:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 67: 9C4-Nivolumab IgG1-KIH-LALA C-Terminal scFv PD1 Monovalent, sAg Bivalent Bispecific 1

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG QVQLVESGGGVVQPGRSLRLDCKASGITFSNSGMHWVR
QAPGKGLEWVAVIWYDGSKRYYADSVK GRFTISRDNSKNTLFLQMNSLR
AEDTAVYYCATNDDYWGQGTLVTVSSGGGGSGGGGSGGGGSEI VLTQSP
ATLSLSPGERATLSCRASQSVSSYLAWYQQKPGQAPRLLIYDASNRATGI
PARFSGSGS GTDFTLTISSLEPEDFAVYYCQQSSNWPRTFGQGTKVEIK

### > Seq 68: 9C4-Nivolumab IgG1-KIH-LALA C-Terminal scFv PD1 Monovalent, sAg Bivalent Bispecific 1

> Heavy Chain 2:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 69: 9C4-Nivolumab (EpCAM-PD-1)IgG1-KIH-LALA C-Terminal scFv PD1 Monovalent, sAg Bivalent Bispecific 1

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 70: 9C4-Nivolumab IgG1-KIH-LALA C-Terminal scFv PD1 Monovalent, sAg Bivalent Bispecific 2

> Heavy Chain 1:

QIQLVQSGPELKKPGETVKISCKASGYTFTNYLLNWVKQAPGKGLKWMG
WINTYTGEPTYTDDFK GRFAFSLATSASTAYLQINNLKNEDTATYFCVR
LAGTKDYWGQGTTLTVSSASTKGPSVFPLAPS SKSTSGGTAALGCLVKD
YFPEPVTVSWNSGALTSGVHTFPAVLQSSGLYSLSSVVTVPSSSLGTQ T
YICNVNHKPSNTKVDKKVEPKSCDKTHTCPPCPAPEAAGGPSVFLFPPKP
KDTLMISRTPEVTC VVVDVSHEDPEVKFNWYVDGVEVHNAKTKPREEQY
NSTYRVVSVLTVLHQDWLNGKEYKCKVSNK ALPAPIEKTISKAKGQPRE
PQVYTLPPSRDELTKNQVSLWCLVKGFYPSDIAVEWESNGQPENNY KTT
PPVLDSDGSFFLYSKLTVDKSRWQQGNVFSCSVMHEALHNHYTQKSLSLS
PGKGGGGAGGGG EIVLTQSPATLSLSPGERATLSCRASQSVSSYLAWYQ
QKPGQAPRLLIYDASNRATGIPARFSGS GSGTDFTLTISSLEPEDFAVY
YCQQSSNWPRTFGQGTKVEIKGGGGSGGGGSGGGGSQVQLVESG GGVVQ
PGRSLRLDCKASGITFSNSGMHWVRQAPGKGLEWVAVIWYDGSKRYYADS
VKGRFTISRD NSKNTLFLQMNSLRAEDTAVYYCATNDDYWGQGTLVTVS
S

### > Seq 70: 9C4-Nivolumab IgG1-KIH-LALA C-Terminal scFv PD1 Monovalent, sAg Bivalent Bispecific 2

> Heavy Chain 2:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 71: 9C4-Nivolumab (EpCAM-PD-1)IgG1-KIH-LALA C-Terminal scFv PD1 Monovalent, sAg Bivalent Bispecific 2

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 72: 9C4-Nivolumab IgG1-KIH-LALA C-Terminal scFv PD1 Monovalent, sAg Bivalent Bispecific 3

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG QVQLVESGGGVVQPGRSLRLDCKASGITFSNSGMHWVR
QAPGKGLEWVAVIWYDGSKRYYADSVK GRFTISRDNSKNTLFLQMNSLR
AEDTAVYYCATNDDYWGQGTLVTVSSGGGGSGGGGSGGGGSEI VLTQSP
ATLSLSPGERATLSCRASQSVSSYLAWYQQKPGQAPRLLIYDASNRATGI
PARFSGSGS GTDFTLTISSLEPEDFAVYYCQQSSNWPRTFGQGTKVEIK

### > Seq 73: 9C4-Nivolumab IgG1-KIH-LALA C-Terminal scFv PD1 Monovalent, sAg Bivalent Bispecific 3

> Heavy Chain 2:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 74: 9C4-Nivolumab (EpCAM-PD-1)IgG1-KIH-LALA C-Terminal scFv PD1 Monovalent, sAg Bivalent Bispecific 3

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 75: 9C4-Nivolumab IgG1-KIH-LALA C-Terminal scFv PD1 Monovalent, sAg Bivalent Bispecific 4

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG EIVLTQSPATLSLSPGERATLSCRASQSVSSYLAWYQQ
KPGQAPRLLIYDASNRATGIPARFSGS GSGTDFTLTISSLEPEDFAVYY
CQQSSNWPRTFGQGTKVEIKGGGGSGGGGSGGGGSQVQLVESG GGVVQP
GRSLRLDCKASGITFSNSGMHWVRQAPGKGLEWVAVIWYDGSKRYYADSV
KGRFTISRD NSKNTLFLQMNSLRAEDTAVYYCATNDDYWGQGTLVTVSS

### > Seq 76: 9C4-Nivolumab (EpCAM-PD-1)IgG1-KIH-LALA C-Terminal scFv PD1 Monovalent, sAg Bivalent Bispecific 4

> Heavy Chain 2:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 77: 9C4-Nivolumab (EpCAM-PD-1)IgG1-KIH-LALA C-Terminal scFv PD1 Monovalent, sAg Bivalent Bispecific 4

> Light Chain:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 78: 9C4-Nivolumab IgG1-KIH-LALA Monovalent Bispecific 1

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 79: 9C4-Nivolumab (EpCAM-PD-1)IgG1-KIH-LALA Monovalent Bispecific 1

> Heavy Chain 2:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPSSKS TSGGTAALGCLVKDYFPE
PVTVSWNSGALTSGVHTFPAVLQSSGLYSLSSVVTVPSSSLGTQTYI CN
VNHKPSNTKVDKKVEPKSCDKTHTCPPCPAPEAAGGPSVFLFPPKPKDTL
MISRTPEVTCVVV DVSHEDPEVKFNWYVDGVEVHNAKTKPREEQYNSTY
RVVSVLTVLHQDWLNGKEYKCKVSNKALP APIEKTISKAKGQPREPQVY
TLPPSRDELTKNQVSLWCLVKGFYPSDIAVEWESNGQPENNYKTT PPVL
DSDGSFFLYSKLTVDKSRWQQGNVFSCSVMHEALHNHYTQKSLSLSPGK

### > Seq 80: 9C4-Nivolumab (EpCAM-PD-1)IgG1-KIH-LALA Monovalent Bispecific 1

> Light Chain 1:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 81: 9C4-Nivolumab (EpCAM-PD-1)IgG1-KIH-LALA Monovalent Bispecific 1

> Light Chain 2:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 82: 9C4-Nivolumab IgG1-KIH-LALA Monovalent Bispecific 2

> Heavy Chain 1:

QIQLVQSGPELKKPGETVKISCKASGYTFTNYLLNWVKQAPGKGLKWMGW
INTYTGEPTYTDDFK GRFAFSLATSASTAYLQINNLKNEDTATYFCVRL
AGTKDYWGQGTTLTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 83: 9C4-Nivolumab (EpCAM-PD-1)IgG1-KIH-LALA Monovalent Bispecific 2

> Heavy Chain 2:

QVQLVESGGGVVQPGRSLRLDCKASGITFSNSGMHWVRQAPGKGLEWVA
VIWYDGSKRYYADSVK GRFTISRDNSKNTLFLQMNSLRAEDTAVYYCAT
NDDYWGQGTLVTVSSASTKGPSVFPLAPSSKS TSGGTAALGCLVKDYFP
EPVTVSWNSGALTSGVHTFPAVLQSSGLYSLSSVVTVPSSSLGTQTYI C
NVNHKPSNTKVDKKVEPKSCDKTHTCPPCPAPEAAGGPSVFLFPPKPKDT
LMISRTPEVTCVVV DVSHEDPEVKFNWYVDGVEVHNAKTKPREEQYNST
YRVVSVLTVLHQDWLNGKEYKCKVSNKALP APIEKTISKAKGQPREPQV
YTLPPSRDELTKNQVSLWCLVKGFYPSDIAVEWESNGQPENNYKTT PPV
LDSDGSFFLYSKLTVDKSRWQQGNVFSCSVMHEALHNHYTQKSLSLSPGK

### > Seq 84: 9C4-Nivolumab (EpCAM-PD-1)IgG1-KIH-LALA Monovalent Bispecific 2

> Light Chain 1:

DIVMTQAAFSNPVTLGTSASISCRSSKSLLHSNGITYLYWYLQKPGQSPQ
LLIYQMSNLASGVPD RFSSSGSGTDFTLRISRVEAEDVGVYYCAQNLEL
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 85: 9C4-Nivolumab (EpCAM-PD-1)IgG1-KIH-LALA Monovalent Bispecific 2

> Light Chain 2:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 86: MOC31-Pembrolizumab (EpCAM-PD-1)IgG4-SPLE C-Terminal scFv Bivalent Bispecific sAg-PD-1 scFv Orientation 1

> Heavy Chain:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPC SRSTSESTAALGCLVKDY
FPEPVTVSWNSGALTSGVHTFPAVLQSSGLYSLSSVVTVPSSSLGTK TY
TCNVDHKPSNTKVDKRVESKYGPPCPSCPAPEFLGGPSVFLFPPKPKDTL
MISRTPEVTCVVV DVSQEDPEVQFNWYVDGVEVHNAKTKPREEQFNSTY
RVVSVLTVLHQDWLNGKEYKCKVSNKGLP SSIEKTISKAKGQPREPQVY
TLPPSQEEMTKNQVSLTCLVKGFYPSDIAVEWESNGQPENNYKTT PPVL
DSDGSFFLYSRLTVDKSRWQEGNVFSCSVMHEALHNHYTQKSLSLSLGKG
GGGAGGGGQVQ LVQSGVEVKKPGASVKVSCKASGYTFTNYYMYWVRQAP
GQGLEWMJIGGINPSNGGTNFNEKFKNRV TLTTDSSTTTAYMELKSLQF
DDTAVYYCARRDYRFDMGFDYWGQGTTVTVSSGGGGSGGGGSGGG GSEI
VLTQSPATLSLSPGERATLSCRASKGVSTSGYSYLHWYQQKPGQAPRLLI
YLASYLESGVP ARFSGSGSGTDFTLTISSLEPEDFAVYYCQHSRDLPLT
FGGGTKVEIK

### > Seq 87: MOC31-Pembrolizumab (EpCAM-PD-1)IgG4-SPLE C-Terminal scFv Bivalent Bispecific sAg-PD-1 scFv Orientation 1

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 88: MOC31-Pembrolizumab IgG4-SPLE C-Terminal scFv Bivalent Bispecific sAg-PD-1 scFv Orientation 2

> Heavy Chain:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPC SRSTSESTAALGCLVKDY
FPEPVTVSWNSGALTSGVHTFPAVLQSSGLYSLSSVVTVPSSSLGTK TY
TCNVDHKPSNTKVDKRVESKYGPPCPSCPAPEFLGGPSVFLFPPKPKDTL
MISRTPEVTCVVV DVSQEDPEVQFNWYVDGVEVHNAKTKPREEQFNSTY
RVVSVLTVLHQDWLNGKEYKCKVSNKGLP SSIEKTISKAKGQPREPQVY
TLPPSQEEMTKNQVSLTCLVKGFYPSDIAVEWESNGQPENNYKTT PPVL
DSDGSFFLYSRLTVDKSRWQEGNVFSCSVMHEALHNHYTQKSLSLSLGKG
GGGAGGGGEIV LTQSPATLSLSPGERATLSCRASKGVSTSGYSYLHWYQ
QKPGQAPRLLIYLASYLESGVPARFSG SGSGTDFTLTISSLEPEDFAVY
YCQHSRDLPLTFGGGTKVEIKGGGGSGGGGSGGGGSQVQLVQS GVEVKK
PGASVKVSCKASGYTFTNYYMYWVRQAPGQGLEWMGGINPSNGGTNFNEK
FKNRVTLTT DSSTTTAYMELKSLQFDDTAVYYCARRDYRFDMGFDYWGQ
GTTVTVSS

### > Seq 89: MOC31-Pembrolizumab (EpCAM-PD-1)IgG4-SPLE C-Terminal scFv Bivalent Bispecific sAg-PD-1 scFv Orientation 2

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 90: MOC31-Pembrolizumab IgG4-SPLE C-Terminal scFv Bivalent Bispecific PD-1-sAg scFv Orientation 1

> Heavy Chain:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPCSRSTSESTAALGCL
VKDYFPEPVTVSWNSGALTSGVHTFPAVLQSSGLYSLSSVVTVPSSS LG
TKTYTCNVDHKPSNTKVDKRVESKYGPPCPSCPAPEFLGGPSVFLFPPKP
KDTLMISRTPEVT CVVVDVSQEDPEVQFNWYVDGVEVHNAKTKPREEQF
NSTYRVVSVLTVLHQDWLNGKEYKCKVSN KGLPSSIEKTISKAKGQPRE
PQVYTLPPSQEEMTKNQVSLTCLVKGFYPSDIAVEWESNGQPENN YKTT
PPVLDSDGSFFLYSRLTVDKSRWQEGNVFSCSVMHEALHNHYTQKSLSLS
LGKGGGGAGGG GQVKLQQSGPELKKPGETVKISCKASGYTFTNYGMNWV
KQAPGKGLKWMGWINTYTGESTYADDF KGRFAFSLETSASAAYLQINNL
KNEDTATYFCARFAIKGDYWGQGTTVTVSSGGGGSGGGGSGGG GSDIVL
TQSPFSNPVTLGTSASISCRSTKSLLHSNGITYLYWYLQKPGQSPQLLIY
QMSNLASGV PDRFSSSGSGTDFTLRISRVEAEDVGVYYCAQNLEIPRTF
GGGTKLEIK

### > Seq 91: MOC31-Pembrolizumab (EpCAM-PD-1)IgG4-SPLE C-Terminal scFv Bivalent Bispecific PD-1-sAg scFv Orientation 1

> Light Chain:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 92: MOC31-Pembrolizumab IgG4-SPLE C-Terminal scFv Bivalent Bispecific PD1-sAg scFv Orientation 2

> Heavy Chain:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPCSRSTSESTAALGCL
VKDYFPEPVTVSWNSGALTSGVHTFPAVLQSSGLYSLSSVVTVPSSS LG
TKTYTCNVDHKPSNTKVDKRVESKYGPPCPSCPAPEFLGGPSVFLFPPKP
KDTLMISRTPEVT CVVVDVSQEDPEVQFNWYVDGVEVHNAKTKPREEQF
NSTYRVVSVLTVLHQDWLNGKEYKCKVSN KGLPSSIEKTISKAKGQPRE
PQVYTLPPSQEEMTKNQVSLTCLVKGFYPSDIAVEWESNGQPENN YKTT
PPVLDSDGSFFLYSRLTVDKSRWQEGNVFSCSVMHEALHNHYTQKSLSLS
LGKGGGGAGGG GDIVLTQSPFSNPVTLGTSASISCRSTKSLLHSNGITY
LYWYLQKPGQSPQLLIYQMSNLASGVP DRFSSSGSGTDFTLRISRVEAE
DVGVYYCAQNLEIPRTFGGGTKLEIKGGGGSGGGGSGGGGSQV KLQQSG
PELKKPGETVKISCKASGYTFTNYGMNWVKQAPGKGLKWMGWINTYTGES
TYADDFKGR FAFSLETSASAAYLQINNLKNEDTATYFCARFAIKGDYWG
QGTTVTVSS

### > Seq 93: MOC31-Pembrolizumab (EpCAM-PD-1)IgG4-SPLE C-Terminal scFv Bivalent Bispecific PD1-sAg scFv Orientation 2

> Light Chain:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 94: MOC31-Pembrolizumab IgG1-KIH-LALA C-Terminal scFv PD1 Monovalent, sAg Bivalent Bispecific 1

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG QVQLVQSGVEVKKPGASVKVSCKASGYTFTNYYMYWVR
QAPGQGLEWMGGINPSNGGTNFNEKFK NRVTLTTDSSTTTAYMELKSLQ
FDDTAVYYCARRDYRFDMGFDYWGQGTTVTVSSGGGGSGGGGS GGGGSE
IVLTQSPATLSLSPGERATLSCRASKGVSTSGYSYLHWYQQKPGQAPRLL
IYLASYLES GVPARFSGSGSGTDFTLTISSLEPEDFAVYYCQHSRDLPL
TFGGGTKVEIK

### > Seq 95: MOC31-Pembrolizumab IgG1-KIH-LALA C-Terminal scFv PD1 Monovalent, sAg Bivalent Bispecific 1

> Heavy Chain 2:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 96: MOC31-Pembrolizumab (EpCAM-PD-1)IgG1-KIH-LALA C-Terminal scFv PD1 Monovalent, sAg Bivalent Bispecific 1

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 97: MOC31-Pembrolizumab IgG1-KIH-LALA C-Terminal scFv PD1 Monovalent, sAg Bivalent Bispecific 2

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG EIVLTQSPATLSLSPGERATLSCRASKGVSTSGYSYLH
WYQQKPGQAPRLLIYLASYLESGVPAR FSGSGSGTDFTLTISSLEPEDF
AVYYCQHSRDLPLTFGGGTKVEIKGGGGSGGGGSGGGGSQVQL VQSGVE
VKKPGASVKVSCKASGYTFTNYYMYWVRQAPGQGLEWMGGINPSNGGTNF
NEKFKNRVT LTTDSSTTTAYMELKSLQFDDTAVYYCARRDYRFDMGFDY
WGQGTTVTVSS

### > Seq 97: MOC31-Pembrolizumab IgG1-KIH-LALA C-Terminal scFv PD1 Monovalent, sAg Bivalent Bispecific 2

> Heavy Chain 2:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 98: MOC31-Pembrolizumab (EpCAM-PD-1)IgG1-KIH-LALA C-Terminal scFv PD1 Monovalent, sAg Bivalent Bispecific 2

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 99: MOC31-Pembrolizumab IgG1-KIH-LALA C-Terminal scFv PD1 Monovalent, sAg Bivalent Bispecific 3

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG QVQLVQSGVEVKKPGASVKVSCKASGYTFTNYYMYWVR
QAPGQGLEWMGGINPSNGGTNFNEKFK NRVTLTTDSSTTTAYMELKSLQ
FDDTAVYYCARRDYRFDMGFDYWGQGTTVTVSSGGGGSGGGGS GGGGSE
IVLTQSPATLSLSPGERATLSCRASKGVSTSGYSYLHWYQQKPGQAPRLL
IYLASYLES GVPARFSGSGSGTDFTLTISSLEPEDFAVYYCQHSRDLPL
TFGGGTKVEIK

### > Seq 100: MOC31-Pembrolizumab IgG1-KIH-LALA C-Terminal scFv PD1 Monovalent, sAg Bivalent Bispecific 3

> Heavy Chain 2:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 101: MOC31-Pembrolizumab (EpCAM-PD-1)IgG1-KIH-LALA C-Terminal scFv PD1 Monovalent, sAg Bivalent Bispecific 3

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 102: MOC31-Pembrolizumab IgG1-KIH-LALA C-Terminal scFv PD1 Monovalent, sAg Bivalent Bispecific 4

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG EIVLTQSPATLSLSPGERATLSCRASKGVSTSGYSYLH
WYQQKPGQAPRLLIYLASYLESGVPAR FSGSGSGTDFTLTISSLEPEDF
AVYYCQHSRDLPLTFGGGTKVEIKGGGGSGGGGSGGGGSQVQL VQSGVE
VKKPGASVKVSCKASGYTFTNYYMYWVRQAPGQGLEWMGGINPSNGGTNF
NEKFKNRVT LTTDSSTTTAYMELKSLQFDDTAVYYCARRDYRFDMGFDY
WGQGTTVTVSS

### > Seq 103: MOC31-Pembrolizumab (EpCAM-PD-1)IgG1-KIH-LALA C-Terminal scFv PD1 Monovalent, sAg Bivalent Bispecific 4

> Heavy Chain 2:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 104: MOC31-Pembrolizumab (EpCAM-PD-1)IgG1-KIH-LALA C-Terminal scFv PD1 Monovalent, sAg Bivalent Bispecific 4

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 105: MOC31-Pembrolizumab IgG1-KIH-LALA Monovalent Bispecific 1

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 106: MOC31-Pembrolizumab (EpCAM-PD-1)IgG1-KIH-LALA Monovalent Bispecific 1

> Heavy Chain 2:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPSSKSTSGGTAALGCL
VKDYFPEPVTVSWNSGALTSGVHTFPAVLQSSGLYSLSSVVTVPSSS LG
TQTYICNVNHKPSNTKVDKKVEPKSCDKTHTCPPCPAPEAAGGPSVFLFP
PKPKDTLMISRTP EVTCVVVDVSHEDPEVKFNWYVDGVEVHNAKTKPRE
EQYNSTYRVVSVLTVLHQDWLNGKEYKCK VSNKALPAPIEKTISKAKGQ
PREPQVYTLPPSRDELTKNQVSLWCLVKGFYPSDIAVEWESNGQP ENNY
KTTPPVLDSDGSFFLYSKLTVDKSRWQQGNVFSCSVMHEALHNHYTQKSL
SLSPGK

### > Seq 107: MOC31-Pembrolizumab (EpCAM-PD-1)IgG1-KIH-LALA Monovalent Bispecific 1

> Light Chain 1:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 108: MOC31-Pembrolizumab (EpCAM-PD-1)IgG1-KIH-LALA Monovalent Bispecific 1

> Light Chain 2:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 109: MOC31-Pembrolizumab IgG1-KIH-LALA Monovalent Bispecific 2

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 110: MOC31-Pembrolizumab (EpCAM-PD-1)IgG1-KIH-LALA Monovalent Bispecific 2

> Heavy Chain 2:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPSSKSTSGGTAALGCL
VKDYFPEPVTVSWNSGALTSGVHTFPAVLQSSGLYSLSSVVTVPSSS LG
TQTYICNVNHKPSNTKVDKKVEPKSCDKTHTCPPCPAPEAAGGPSVFLFP
PKPKDTLMISRTP EVTCVVVDVSHEDPEVKFNWYVDGVEVHNAKTKPRE
EQYNSTYRVVSVLTVLHQDWLNGKEYKCK VSNKALPAPIEKTISKAKGQ
PREPQVYTLPPSRDELTKNQVSLWCLVKGFYPSDIAVEWESNGQP ENNY
KTTPPVLDSDGSFFLYSKLTVDKSRWQQGNVFSCSVMHEALHNHYTQKSL
SLSPGK

### > Seq 111: MOC31-Pembrolizumab (EpCAM-PD-1)IgG1-KIH-LALA Monovalent Bispecific 2

> Light Chain 1:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 112: MOC31-Pembrolizumab (EpCAM-PD-1)IgG1-KIH-LALA Monovalent Bispecific 2

> Light Chain 2:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 113: MOC31-Nivolumab (EpCAM-PD-1)IgG4-SPLE C-Terminal scFv Bivalent Bispecific sAg-PD-1 scFv Orientation 1

> Heavy Chain:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPC SRSTSESTAALGCLVKDY
FPEPVTVSWNSGALTSGVHTFPAVLQSSGLYSLSSVVTVPSSSLGTK TY
TCNVDHKPSNTKVDKRVESKYGPPCPSCPAPEFLGGPSVFLFPPKPKDTL
MISRTPEVTCVVV DVSQEDPEVQFNWYVDGVEVHNAKTKPREEQFNSTY
RVVSVLTVLHQDWLNGKEYKCKVSNKGLP SSIEKTISKAKGQPREPQVY
TLPPSQEEMTKNQVSLTCLVKGFYPSDIAVEWESNGQPENNYKTT PPVL
DSDGSFFLYSRLTVDKSRWQEGNVFSCSVMHEALHNHYTQKSLSLSLGKG
GGGAGGGGQVQ LVESGGGVVQPGRSLRLDCKASGITFSNSGMHWVRQAP
GKGLEWVAVIWYDGSKRYYADSVKGRF TISRDNSKNTLFLQMNSLRAED
TAVYYCATNDDYWGQGTLVTVSSGGGGSGGGGSGGGGSEIVLT QSPATL
SLSPGERATLSCRASQSVSSYLAWYQQKPGQAPRLLIYDASNRATGIPAR
FSGSGSGTD FTLTISSLEPEDFAVYYCQQSSNWPRTFGQGTKVEIK

### > Seq 114: MOC31-Nivolumab (EpCAM-PD-1)IgG4-SPLE C-Terminal scFv Bivalent Bispecific sAg-PD-1 scFv Orientation 1

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 115: MOC31-Nivolumab IgG4-SPLE C-Terminal scFv Bivalent Bispecific sAg-PD-1 scFv Orientation 2

> Heavy Chain:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPC SRSTSESTAALGCLVKDY
FPEPVTVSWNSGALTSGVHTFPAVLQSSGLYSLSSVVTVPSSSLGTK TY
TCNVDHKPSNTKVDKRVESKYGPPCPSCPAPEFLGGPSVFLFPPKPKDTL
MISRTPEVTCVVV DVSQEDPEVQFNWYVDGVEVHNAKTKPREEQFNSTY
RVVSVLTVLHQDWLNGKEYKCKVSNKGLP SSIEKTISKAKGQPREPQVY
TLPPSQEEMTKNQVSLTCLVKGFYPSDIAVEWESNGQPENNYKTT PPVL
DSDGSFFLYSRLTVDKSRWQEGNVFSCSVMHEALHNHYTQKSLSLSLGKG
GGGAGGGGEIV LTQSPATLSLSPGERATLSCRASQSVSSYLAWYQQKPG
QAPRLLIYDASNRATGIPARFSGSGSG TDFTLTISSLEPEDFAVYYCQQ
SSNWPRTFGQGTKVEIKGGGGSGGGGSGGGGSQVQLVESGGGV VQPGRS
LRLDCKASGITFSNSGMHWVRQAPGKGLEWVAVIWYDGSKRYYADSVKGR
FTISRDNSK NTLFLQMNSLRAEDTAVYYCATNDDYWGQGTLVTVSS

### > Seq 116: MOC31-Nivolumab (EpCAM-PD-1)IgG4-SPLE C-Terminal scFv Bivalent Bispecific sAg-PD-1 scFv Orientation 2

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 117: MOC31-Nivolumab IgG4-SPLE C-Terminal scFv Bivalent Bispecific PD-1-sAg scFv Orientation 1

> Heavy Chain:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPCSRS TSESTAALGCLVKDYFPE
PVTVSWNSGALTSGVHTFPAVLQSSGLYSLSSVVTVPSSSLGTKTYT CN
VDHKPSNTKVDKRVESKYGPPCPSCPAPEFLGGPSVFLFPPKPKDTLMIS
RTPEVTCVVVDVS QEDPEVQFNWYVDGVEVHNAKTKPREEQFNSTYRVV
SVLTVLHQDWLNGKEYKCKVSNKGLPSSI EKTISKAKGQPREPQVYTLP
PSQEEMTKNQVSLTCLVKGFYPSDIAVEWESNGQPENNYKTTPPV LDSD
GSFFLYSRLTVDKSRWQEGNVFSCSVMHEALHNHYTQKSLSLSLGKGGGG
AGGGGQVKLQQ SGPELKKPGETVKISCKASGYTFTNYGMNWVKQAPGKG
LKWMGWINTYTGESTYADDFKGRFAFS LETSASAAYLQINNLKNEDTAT
YFCARFAIKGDYWGQGTTVTVSSGGGGSGGGGSGGGGSDIVLT QSPFSN
PVTLGTSASISCRSTKSLLHSNGITYLYWYLQKPGQSPQLLIYQMSNLAS
GVPDRFSSS GSGTDFTLRISRVEAEDVGVYYCAQNLEIPRTFGGGTKLE
IK

### > Seq 118: MOC31-Nivolumab (EpCAM-PD-1)IgG4-SPLE C-Terminal scFv Bivalent Bispecific PD-1-sAg scFv Orientation 1

> Light Chain:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 119: MOC31-Nivolumab IgG4-SPLE C-Terminal scFv Bivalent Bispecific PD1-sAg scFv Orientation 2

> Heavy Chain:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPCSRS TSESTAALGCLVKDYFPE
PVTVSWNSGALTSGVHTFPAVLQSSGLYSLSSVVTVPSSSLGTKTYT CN
VDHKPSNTKVDKRVESKYGPPCPSCPAPEFLGGPSVFLFPPKPKDTLMIS
RTPEVTCVVVDVS QEDPEVQFNWYVDGVEVHNAKTKPREEQFNSTYRVV
SVLTVLHQDWLNGKEYKCKVSNKGLPSSI EKTISKAKGQPREPQVYTLP
PSQEEMTKNQVSLTCLVKGFYPSDIAVEWESNGQPENNYKTTPPV LDSD
GSFFLYSRLTVDKSRWQEGNVFSCSVMHEALHNHYTQKSLSLSLGKGGGG
AGGGGDIVLTQ SPFSNPVTLGTSASISCRSTKSLLHSNGITYLYWYLQK
PGQSPQLLIYQMSNLASGVPDRFSSSG SGTDFTLRISRVEAEDVGVYYC
AQNLEIPRTFGGGTKLEIKGGGGSGGGGSGGGGSQVKLQQSGP ELKKPG
ETVKISCKASGYTFTNYGMNWVKQAPGKGLKWMGWINTYTGESTYADDFK
GRFAFSLET SASAAYLQINNLKNEDTATYFCARFAIKGDYWGQGTTVTV
SS

### > Seq 120: MOC31-Nivolumab (EpCAM-PD-1)IgG4-SPLE C-Terminal scFv Bivalent Bispecific PD1-sAg scFv Orientation 2

> Light Chain:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 121: MOC31-Nivolumab IgG1-KIH-LALA C-Terminal scFv PD1 Monovalent, sAg Bivalent Bispecific 1

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG QVQLVESGGGVVQPGRSLRLDCKASGITFSNSGMHWVR
QAPGKGLEWVAVIWYDGSKRYYADSVK GRFTISRDNSKNTLFLQMNSLR
AEDTAVYYCATNDDYWGQGTLVTVSSGGGGSGGGGSGGGGSEI VLTQSP
ATLSLSPGERATLSCRASQSVSSYLAWYQQKPGQAPRLLIYDASNRATGI
PARFSGSGS GTDFTLTISSLEPEDFAVYYCQQSSNWPRTFGQGTKVEIK

### > Seq 122: MOC31-Nivolumab IgG1-KIH-LALA C-Terminal scFv PD1 Monovalent, sAg Bivalent Bispecific 1

> Heavy Chain 2:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 123: MOC31-Nivolumab (EpCAM-PD-1)IgG1-KIH-LALA C-Terminal scFv PD1 Monovalent, sAg Bivalent Bispecific 1

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 124: MOC31-Nivolumab IgG1-KIH-LALA C-Terminal scFv PD1 Monovalent, sAg Bivalent Bispecific 2

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG EIVLTQSPATLSLSPGERATLSCRASQSVSSYLAWYQQ
KPGQAPRLLIYDASNRATGIPARFSGS GSGTDFTLTISSLEPEDFAVYY
CQQSSNWPRTFGQGTKVEIKGGGGSGGGGSGGGGSQVQLVESG GGVVQP
GRSLRLDCKASGITFSNSGMHWVRQAPGKGLEWVAVIWYDGSKRYYADSV
KGRFTISRD NSKNTLFLQMNSLRAEDTAVYYCATNDDYWGQGTLVTVSS

### > Seq 124: MOC31-Nivolumab IgG1-KIH-LALA C-Terminal scFv PD1 Monovalent, sAg Bivalent Bispecific 2

> Heavy Chain 2:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 125: MOC31-Nivolumab (EpCAM-PD-1)IgG1-KIH-LALA C-Terminal scFv PD1 Monovalent, sAg Bivalent Bispecific 2

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 126: MOC31-Nivolumab IgG1-KIH-LALA C-Terminal scFv PD1 Monovalent, sAg Bivalent Bispecific 3

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG QVQLVESGGGVVQPGRSLRLDCKASGITFSNSGMHWVR
QAPGKGLEWVAVIWYDGSKRYYADSVK GRFTISRDNSKNTLFLQMNSLR
AEDTAVYYCATNDDYWGQGTLVTVSSGGGGSGGGGSGGGGSEI VLTQSP
ATLSLSPGERATLSCRASQSVSSYLAWYQQKPGQAPRLLIYDASNRATGI
PARFSGSGS GTDFTLTISSLEPEDFAVYYCQQSSNWPRTFGQGTKVEIK

### > Seq 127: MOC31-Nivolumab IgG1-KIH-LALA C-Terminal scFv PD1 Monovalent, sAg Bivalent Bispecific 3

> Heavy Chain 2:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 128: MOC31-Nivolumab (EpCAM-PD-1)IgG1-KIH-LALA C-Terminal scFv PD1 Monovalent, sAg Bivalent Bispecific 3

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 129: MOC31-Nivolumab IgG1-KIH-LALA C-Terminal scFv PD1 Monovalent, sAg Bivalent Bispecific 4

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GKGGGGAGGGG EIVLTQSPATLSLSPGERATLSCRASQSVSSYLAWYQQ
KPGQAPRLLIYDASNRATGIPARFSGS GSGTDFTLTISSLEPEDFAVYY
CQQSSNWPRTFGQGTKVEIKGGGGSGGGGSGGGGSQVQLVESG GGVVQP
GRSLRLDCKASGITFSNSGMHWVRQAPGKGLEWVAVIWYDGSKRYYADSV
KGRFTISRD NSKNTLFLQMNSLRAEDTAVYYCATNDDYWGQGTLVTVSS

### > Seq 130: MOC31-Nivolumab (EpCAM-PD-1)IgG1-KIH-LALA C-Terminal scFv PD1 Monovalent, sAg Bivalent Bispecific 4

> Heavy Chain 2:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 131: MOC31-Nivolumab (EpCAM-PD-1)IgG1-KIH-LALA C-Terminal scFvPD1 Monovalent, sAg Bivalent Bispecific 4

> Light Chain:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 132: MOC31-Nivolumab IgG1-KIH-LALA Monovalent Bispecific 1

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 133: MOC31-Nivolumab (EpCAM-PD-1)IgG1-KIH-LALA Monovalent Bispecific 1

> Heavy Chain 2:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPSSKS TSGGTAALGCLVKDYFPE
PVTVSWNSGALTSGVHTFPAVLQSSGLYSLSSVVTVPSSSLGTQTYI CN
VNHKPSNTKVDKKVEPKSCDKTHTCPPCPAPEAAGGPSVFLFPPKPKDTL
MISRTPEVTCVVV DVSHEDPEVKFNWYVDGVEVHNAKTKPREEQYNSTY
RVVSVLTVLHQDWLNGKEYKCKVSNKALP APIEKTISKAKGQPREPQVY
TLPPSRDELTKNQVSLWCLVKGFYPSDIAVEWESNGQPENNYKTT PPVL
DSDGSFFLYSKLTVDKSRWQQGNVFSCSVMHEALHNHYTQKSLSLSPGK

### > Seq 134: MOC31-Nivolumab (EpCAM-PD-1)IgG1-KIH-LALA Monovalent Bispecific 1

> Light Chain 1:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 135: MOC31-Nivolumab (EpCAM-PD-1)IgG1-KIH-LALA Monovalent Bispecific 1

> Light Chain 2:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 136: MOC31-Nivolumab IgG1-KIH-LALA Monovalent Bispecific 2

> Heavy Chain 1:

QVKLQQSGPELKKPGETVKISCKASGYTFTNYGMNWVKQAPGKGLKWMGW
INTYTGESTYADDFK GRFAFSLETSASAAYLQINNLKNEDTATYFCARF
AIKGDYWGQGTTVTVSSASTKGPSVFPLAPS SKSTSGGTAALGCLVKDY
FPEPVTVSWNSGALTSGVHTFPAVLQSSGLYSLSSVVTVPSSSLGTQ TY
ICNVNHKPSNTKVDKKVEPKSCDKTHTCPPCPAPEAAGGPSVFLFPPKPK
DTLMISRTPEVTC VVVDVSHEDPEVKFNWYVDGVEVHNAKTKPREEQYN
STYRVVSVLTVLHQDWLNGKEYKCKVSNK ALPAPIEKTISKAKGQPREP
QVYTLPPSRDELTKNQVSLWCLVKGFYPSDIAVEWESNGQPENNY KTTP
PVLDSDGSFFLYSKLTVDKSRWQQGNVFSCSVMHEALHNHYTQKSLSLSP
GK

### > Seq 137: MOC31-Nivolumab (EpCAM-PD-1)IgG1-KIH-LALA Monovalent Bispecific 2

> Heavy Chain 2:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPSSKS TSGGTAALGCLVKDYFPE
PVTVSWNSGALTSGVHTFPAVLQSSGLYSLSSVVTVPSSSLGTQTYI CN
VNHKPSNTKVDKKVEPKSCDKTHTCPPCPAPEAAGGPSVFLFPPKPKDTL
MISRTPEVTCVVV DVSHEDPEVKFNWYVDGVEVHNAKTKPREEQYNSTY
RVVSVLTVLHQDWLNGKEYKCKVSNKALP APIEKTISKAKGQPREPQVY
TLPPSRDELTKNQVSLWCLVKGFYPSDIAVEWESNGQPENNYKTT PPVL
DSDGSFFLYSKLTVDKSRWQQGNVFSCSVMHEALHNHYTQKSLSLSPGK

### > Seq 138: MOC31-Nivolumab (EpCAM-PD-1)IgG1-KIH-LALA Monovalent Bispecific 2

> Light Chain 1:

DIVLTQSPFSNPVTLGTSASISCRSTKSLLHSNGITYLYWYLQKPGQSPQ
LLIYQMSNLASGVPD RFSSSGSGTDFTLRISRVEAEDVGVYYCAQNLEI
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 139: MOC31-Nivolumab (EpCAM-PD-1)IgG1-KIH-LALA Monovalent Bispecific 2

> Light Chain 2:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 140: Adecatumumab-Pembrolizumab (EpCAM-PD-1)IgG4-SPLE C-Terminal scFv Bivalent Bispecific sAg-PD-1 scFv Orientation 1

> Heavy Chain:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPCSRSTSES
TAALGCLVKDYFPEPVTVSWNSGALTSGVHTFPAVLQSSGLYSLSSV VT
VPSSSLGTKTYTCNVDHKPSNTKVDKRVESKYGPPCPSCPAPEFLGGPSV
FLFPPKPKDTLMI SRTPEVTCVVVDVSQEDPEVQFNWYVDGVEVHNAKT
KPREEQFNSTYRVVSVLTVLHQDWLNGKE YKCKVSNKGLPSSIEKTISK
AKGQPREPQVYTLPPSQEEMTKNQVSLTCLVKGFYPSDIAVEWES NGQP
ENNYKTTPPVLDSDGSFFLYSRLTVDKSRWQEGNVFSCSVMHEALHNHYT
QKSLSLSLGKG GGGAGGGGQVQLVQSGVEVKKPGASVKVSCKASGYTFT
NYYMYWVRQAPGQGLEWMGGINPSNGG TNFNEKFKNRVTLTTDSSTTTA
YMELKSLQFDDTAVYYCARRDYRFDMGFDYWGQGTTVTVSSGG GGSGGG
GSGGGGSEIVLTQSPATLSLSPGERATLSCRASKGVSTSGYSYLHWYQQK
PGQAPRLLI YLASYLESGVPARFSGSGSGTDFTLTISSLEPEDFAVYYC
QHSRDLPLTFGGGTKVEIK

### > Seq 141: Adecatumumab-Pembrolizumab (EpCAM-PD-1)IgG4-SPLE C-Terminal scFv Bivalent Bispecific sAg-PD-1 scFv Orientation 1

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 142: Adecatumumab-Pembrolizumab IgG4-SPLE C-Terminal scFv Bivalent Bispecific sAg-PD-1 scFv Orientation 2

> Heavy Chain:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPCSRSTSES
TAALGCLVKDYFPEPVTVSWNSGALTSGVHTFPAVLQSSGLYSLSSV VT
VPSSSLGTKTYTCNVDHKPSNTKVDKRVESKYGPPCPSCPAPEFLGGPSV
FLFPPKPKDTLMI SRTPEVTCVVVDVSQEDPEVQFNWYVDGVEVHNAKT
KPREEQFNSTYRVVSVLTVLHQDWLNGKE YKCKVSNKGLPSSIEKTISK
AKGQPREPQVYTLPPSQEEMTKNQVSLTCLVKGFYPSDIAVEWES NGQP
ENNYKTTPPVLDSDGSFFLYSRLTVDKSRWQEGNVFSCSVMHEALHNHYT
QKSLSLSLGKG GGGAGGGGEIVLTQSPATLSLSPGERATLSCRASKGVS
TSGYSYLHWYQQKPGQAPRLLIYLASY LESGVPARFSGSGSGTDFTLTI
SSLEPEDFAVYYCQHSRDLPLTFGGGTKVEIKGGGGSGGGGSG GGGSQV
QLVQSGVEVKKPGASVKVSCKASGYTFTNYYMYWVRQAPGQGLEWMGGIN
PSNGGTNFN EKFKNRVTLTTDSSTTTAYMELKSLQFDDTAVYYCARRDY
RFDMGFDYWGQGTTVTVSS

### > Seq 143: Adecatumumab-Pembrolizumab (EpCAM-PD-1)IgG4-SPLE C-Terminal scFv Bivalent Bispecific sAg-PD-1 scFv Orientation 2

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 144: Adecatumumab-Pembrolizumab IgG4-SPLE C-Terminal scFv Bivalent Bispecific PD-1-sAg scFv Orientation 1

> Heavy Chain:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPCSRSTSESTAALGCL
VKDYFPEPVTVSWNSGALTSGVHTFPAVLQSSGLYSLSSVVTVPSSS LG
TKTYTCNVDHKPSNTKVDKRVESKYGPPCPSCPAPEFLGGPSVFLFPPKP
KDTLMISRTPEVT CVVVDVSQEDPEVQFNWYVDGVEVHNAKTKPREEQF
NSTYRVVSVLTVLHQDWLNGKEYKCKVSN KGLPSSIEKTISKAKGQPRE
PQVYTLPPSQEEMTKNQVSLTCLVKGFYPSDIAVEWESNGQPENN YKTT
PPVLDSDGSFFLYSRLTVDKSRWQEGNVFSCSVMHEALHNHYTQKSLSLS
LGKGGGGAGGG GEVQLLESGGGVVQPGRSLRLSCAASGFTFSSYGMHWV
RQAPGKGLEWVAVISYDGSNKYYADSV KGRFTISRDNSKNTLYLQMNSL
RAEDTAVYYCAKDMGWGSGWRPYYYYGMDVWGQGTTVTVSSGG GGSGGG
GSGGGGSELQMTQSPSSLSASVGDRVTITCRTSQSISSYLNWYQQKPGQP
PKLLIYWAS TRESGVPDRFSGSGSGTDFTLTISSLQPEDSATYYCQQSY
DIPYTFGQGTKLEIK

### > Seq 145: Adecatumumab-Pembrolizumab (EpCAM-PD-1)IgG4-SPLE C-Terminal scFv Bivalent Bispecific PD-1-sAg scFv Orientation 1

> Light Chain:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 146: Adecatumumab-Pembrolizumab IgG4-SPLE C-Terminal scFv Bivalent Bispecific PD1-sAg scFv Orientation 2

> Heavy Chain:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPCSRSTSESTAALGCL
VKDYFPEPVTVSWNSGALTSGVHTFPAVLQSSGLYSLSSVVTVPSSS LG
TKTYTCNVDHKPSNTKVDKRVESKYGPPCPSCPAPEFLGGPSVFLFPPKP
KDTLMISRTPEVT CVVVDVSQEDPEVQFNWYVDGVEVHNAKTKPREEQF
NSTYRVVSVLTVLHQDWLNGKEYKCKVSN KGLPSSIEKTISKAKGQPRE
PQVYTLPPSQEEMTKNQVSLTCLVKGFYPSDIAVEWESNGQPENN YKTT
PPVLDSDGSFFLYSRLTVDKSRWQEGNVFSCSVMHEALHNHYTQKSLSLS
LGKGGGGAGGG GELQMTQSPSSLSASVGDRVTITCRTSQSISSYLNWYQ
QKPGQPPKLLIYWASTRESGVPDRFSG SGSGTDFTLTISSLQPEDSATY
YCQQSYDIPYTFGQGTKLEIKGGGGSGGGGSGGGGSEVQLLES GGGVVQ
PGRSLRLSCAASGFTFSSYGMHWVRQAPGKGLEWVAVISYDGSNKYYADS
VKGRFTISR DNSKNTLYLQMNSLRAEDTAVYYCAKDMGWGSGWRPYYYY
GMDVWGQGTTVTVSS

### > Seq 147: Adecatumumab-Pembrolizumab (EpCAM-PD-1)IgG4-SPLE C-Terminal scFv Bivalent Bispecific PD1-sAg scFv Orientation 2

> Light Chain:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 148: Adecatumumab-Pembrolizumab IgG1-KIH-LALA C-Terminal scFv PD1 Monovalent, sAg Bivalent Bispecific 1

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GKGGGGAGGGGQVQLVQSGVEVKKPGASVKVSCKASGY
TFTNYYMYWVRQAPGQGLEWMGGINPS NGGTNFNEKFKNRVTLTTDSST
TTAYMELKSLQFDDTAVYYCARRDYRFDMGFDYWGQGTTVTVS SGGGGS
GGGGSGGGGSEIVLTQSPATLSLSPGERATLSCRASKGVSTSGYSYLHWY
QQKPGQAPR LLIYLASYLESGVPARFSGSGSGTDFTLTISSLEPEDFAV
YYCQHSRDLPLTFGGGTKVEIK

### > Seq 149: Adecatumumab-Pembrolizumab IgG1-KIH-LALA C-Terminal scFv PD1 Monovalent, sAg Bivalent Bispecific 1

> Heavy Chain 2:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 150: Adecatumumab-Pembrolizumab (EpCAM-PD-1)IgG1-KIH-LALA C-Terminal scFv PD1 Monovalent, sAg Bivalent Bispecific 1

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 151: Adecatumumab-Pembrolizumab IgG1-KIH-LALA C-Terminal scFv PD1 Monovalent, sAg Bivalent Bispecific 2

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GKGGGGAGGGGEIVLTQSPATLSLSPGERATLSCRASK
GVSTSGYSYLHWYQQKPGQAPRLLIYL ASYLESGVPARFSGSGSGTDFT
LTISSLEPEDFAVYYCQHSRDLPLTFGGGTKVEIKGGGGSGGG GSGGGG
SQVQLVQSGVEVKKPGASVKVSCKASGYTFTNYYMYWVRQAPGQGLEWMG
GINPSNGGT NFNEKFKNRVTLTTDSSTTTAYMELKSLQFDDTAVYYCAR
RDYRFDMGFDYWGQGTTVTVSS

### > Seq 151: Adecatumumab-Pembrolizumab IgG1-KIH-LALA C-Terminal scFv PD1 Monovalent, sAg Bivalent Bispecific 2

> Heavy Chain 2:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 152: Adecatumumab-Pembrolizumab (EpCAM-PD-1)IgG1-KIH-LALA C-Terminal scFv PD1 Monovalent, sAg Bivalent Bispecific 2

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 153: Adecatumumab-Pembrolizumab IgG1-KIH-LALA C-Terminal scFv PD1 Monovalent, sAg Bivalent Bispecific 3

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GKGGGGAGGGGQVQLVQSGVEVKKPGASVKVSCKASGY
TFTNYYMYWVRQAPGQGLEWMGGINPS NGGTNFNEKFKNRVTLTTDSST
TTAYMELKSLQFDDTAVYYCARRDYRFDMGFDYWGQGTTVTVS SGGGGS
GGGGSGGGGSEIVLTQSPATLSLSPGERATLSCRASKGVSTSGYSYLHWY
QQKPGQAPR LLIYLASYLESGVPARFSGSGSGTDFTLTISSLEPEDFAV
YYCQHSRDLPLTFGGGTKVEIK

### > Seq 154: Adecatumumab-Pembrolizumab IgG1-KIH-LALA C-Terminal scFv PD1 Monovalent, sAg Bivalent Bispecific 3

> Heavy Chain 2:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 155: Adecatumumab-Pembrolizumab (EpCAM-PD-1)IgG1-KIH-LALA C-Terminal scFv PD1 Monovalent, sAg Bivalent Bispecific 3

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 156: Adecatumumab-Pembrolizumab IgG1-KIH-LALA C-Terminal scFv PD1 Monovalent, sAg Bivalent Bispecific 4

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GKGGGGAGGGGEIVLTQSPATLSLSPGERATLSCRASK
GVSTSGYSYLHWYQQKPGQAPRLLIYL ASYLESGVPARFSGSGSGTDFT
LTISSLEPEDFAVYYCQHSRDLPLTFGGGTKVEIKGGGGSGGG GSGGGG
SQVQLVQSGVEVKKPGASVKVSCKASGYTFTNYYMYWVRQAPGQGLEWMG
GINPSNGGT NFNEKFKNRVTLTTDSSTTTAYMELKSLQFDDTAVYYCAR
RDYRFDMGFDYWGQGTTVTVSS

### > Seq 157: Adecatumumab-Pembrolizumab (EpCAM-PD-1)IgG1-KIH-LALA C-Terminal scFv PD1 Monovalent, sAg Bivalent Bispecific 4

> Heavy Chain 2:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 158: Adecatumumab-Pembrolizumab (EpCAM-PD-1)IgG1-KIH-LALA C-Terminal scFv PD1 Monovalent, sAg Bivalent Bispecific 4

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 159: Adecatumumab-Pembrolizumab IgG1-KIH-LALA Monovalent Bispecific 1

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 160: Adecatumumab-Pembrolizumab (EpCAM-PD-1)IgG1-KIH-LALA Monovalent Bispecific 1

> Heavy Chain 2:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPSSKSTSGGTAALGCL
VKDYFPEPVTVSWNSGALTSGVHTFPAVLQSSGLYSLSSVVTVPSSS LG
TQTYICNVNHKPSNTKVDKKVEPKSCDKTHTCPPCPAPEAAGGPSVFLFP
PKPKDTLMISRTP EVTCVVVDVSHEDPEVKFNWYVDGVEVHNAKTKPRE
EQYNSTYRVVSVLTVLHQDWLNGKEYKCK VSNKALPAPIEKTISKAKGQ
PREPQVYTLPPSRDELTKNQVSLWCLVKGFYPSDIAVEWESNGQP ENNY
KTTPPVLDSDGSFFLYSKLTVDKSRWQQGNVFSCSVMHEALHNHYTQKSL
SLSPGK

### > Seq 161: Adecatumumab-Pembrolizumab (EpCAM-PD-1)IgG1-KIH-LALA Monovalent Bispecific 1

> Light Chain 1:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 162: Adecatumumab-Pembrolizumab (EpCAM-PD-1)IgG1-KIH-LALA Monovalent Bispecific 1

> Light Chain 2:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 163: Adecatumumab-Pembrolizumab IgG1-KIH-LALA Monovalent Bispecific 2

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 164: Adecatumumab-Pembrolizumab (EpCAM-PD-1)IgG1-KIH-LALA Monovalent Bispecific 2

> Heavy Chain 2:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPSSKSTSGGTAALGCL
VKDYFPEPVTVSWNSGALTSGVHTFPAVLQSSGLYSLSSVVTVPSSS LG
TQTYICNVNHKPSNTKVDKKVEPKSCDKTHTCPPCPAPEAAGGPSVFLFP
PKPKDTLMISRTP EVTCVVVDVSHEDPEVKFNWYVDGVEVHNAKTKPRE
EQYNSTYRVVSVLTVLHQDWLNGKEYKCK VSNKALPAPIEKTISKAKGQ
PREPQVYTLPPSRDELTKNQVSLWCLVKGFYPSDIAVEWESNGQP ENNY
KTTPPVLDSDGSFFLYSKLTVDKSRWQQGNVFSCSVMHEALHNHYTQKSL
SLSPGK

### > Seq 165: Adecatumumab-Pembrolizumab (EpCAM-PD-1)IgG1-KIH-LALA Monovalent Bispecific 2

> Light Chain 1:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 166: Adecatumumab-Pembrolizumab (EpCAM-PD-1)IgG1-KIH-LALA Monovalent Bispecific 2

> Light Chain 2:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 167: Adecatumumab-Nivolumab (EpCAM-PD-1)IgG4-SPLE C-Terminal scFv Bivalent Bispecific sAg-PD-1 scFv Orientation 1

> Heavy Chain:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPCSRSTSES
TAALGCLVKDYFPEPVTVSWNSGALTSGVHTFPAVLQSSGLYSLSSV VT
VPSSSLGTKTYTCNVDHKPSNTKVDKRVESKYGPPCPSCPAPEFLGGPSV
FLFPPKPKDTLMI SRTPEVTCVVVDVSQEDPEVQFNWYVDGVEVHNAKT
KPREEQFNSTYRVVSVLTVLHQDWLNGKE YKCKVSNKGLPSSIEKTISK
AKGQPREPQVYTLPPSQEEMTKNQVSLTCLVKGFYPSDIAVEWES NGQP
ENNYKTTPPVLDSDGSFFLYSRLTVDKSRWQEGNVFSCSVMHEALHNHYT
QKSLSLSLGKG GGGAGGGGQVQLVESGGGVVQPGRSLRLDCKASGITFS
NSGMHWVRQAPGKGLEWVAVIWYDGSK RYYADSVKGRFTISRDNSKNTL
FLQMNSLRAEDTAVYYCATNDDYWGQGTLVTVSSGGGGSGGGG SGGGGS
EIVLTQSPATLSLSPGERATLSCRASQSVSSYLAWYQQKPGQAPRLLIYD
ASNRATGIP ARFSGSGSGTDFTLTISSLEPEDFAVYYCQQSSNWPRTFG
QGTKVEIK

### > Seq 168: Adecatumumab-Nivolumab (EpCAM-PD-1)IgG4-SPLE C-Terminal scFv Bivalent Bispecific sAg-PD-1 scFv Orientation 1

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 169: Adecatumumab-Nivolumab IgG4-SPLE C-Terminal scFv Bivalent Bispecific sAg-PD-1 scFv Orientation 2

> Heavy Chain:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPCSRSTSES
TAALGCLVKDYFPEPVTVSWNSGALTSGVHTFPAVLQSSGLYSLSSV VT
VPSSSLGTKTYTCNVDHKPSNTKVDKRVESKYGPPCPSCPAPEFLGGPSV
FLFPPKPKDTLMI SRTPEVTCVVVDVSQEDPEVQFNWYVDGVEVHNAKT
KPREEQFNSTYRVVSVLTVLHQDWLNGKE YKCKVSNKGLPSSIEKTISK
AKGQPREPQVYTLPPSQEEMTKNQVSLTCLVKGFYPSDIAVEWES NGQP
ENNYKTTPPVLDSDGSFFLYSRLTVDKSRWQEGNVFSCSVMHEALHNHYT
QKSLSLSLGKG GGGAGGGGEIVLTQSPATLSLSPGERATLSCRASQSVS
SYLAWYQQKPGQAPRLLIYDASNRATG IPARFSGSGSGTDFTLTISSLE
PEDFAVYYCQQSSNWPRTFGQGTKVEIKGGGGSGGGGSGGGGS QVQLVE
SGGGVVQPGRSLRLDCKASGITFSNSGMHWVRQAPGKGLEWVAVIWYDGS
KRYYADSVK GRFTISRDNSKNTLFLQMNSLRAEDTAVYYCATNDDYWGQ
GTLVTVSS

### > Seq 170: Adecatumumab-Nivolumab (EpCAM-PD-1)IgG4-SPLE C-Terminal scFv Bivalent Bispecific sAg-PD-1 scFv Orientation 2

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 171: Adecatumumab-Nivolumab IgG4-SPLE C-Terminal scFv Bivalent Bispecific PD-1-sAg scFv Orientation 1

> Heavy Chain:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPCSRS TSESTAALGCLVKDYFPE
PVTVSWNSGALTSGVHTFPAVLQSSGLYSLSSVVTVPSSSLGTKTYT CN
VDHKPSNTKVDKRVESKYGPPCPSCPAPEFLGGPSVFLFPPKPKDTLMIS
RTPEVTCVVVDVS QEDPEVQFNWYVDGVEVHNAKTKPREEQFNSTYRVV
SVLTVLHQDWLNGKEYKCKVSNKGLPSSI EKTISKAKGQPREPQVYTLP
PSQEEMTKNQVSLTCLVKGFYPSDIAVEWESNGQPENNYKTTPPV LDSD
GSFFLYSRLTVDKSRWQEGNVFSCSVMHEALHNHYTQKSLSLSLGKGGGG
AGGGGEVQLLE SGGGVVQPGRSLRLSCAASGFTFSSYGMHWVRQAPGKG
LEWVAVISYDGSNKYYADSVKGRFTIS RDNSKNTLYLQMNSLRAEDTAV
YYCAKDMGWGSGWRPYYYYGMDVWGQGTTVTVSSGGGGSGGGG SGGGGS
ELQMTQSPSSLSASVGDRVTITCRTSQSISSYLNWYQQKPGQPPKLLIYW
ASTRESGVP DRFSGSGSGTDFTLTISSLQPEDSATYYCQQSYDIPYTFG
QGTKLEIK

### > Seq 172: Adecatumumab-Nivolumab (EpCAM-PD-1)IgG4-SPLE C-Terminal scFv Bivalent Bispecific PD-1-sAg scFv Orientation 1

> Light Chain:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 173: Adecatumumab-Nivolumab IgG4-SPLE C-Terminal scFv Bivalent Bispecific PD1-sAg scFv Orientation 2

> Heavy Chain:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPCSRS TSESTAALGCLVKDYFPE
PVTVSWNSGALTSGVHTFPAVLQSSGLYSLSSVVTVPSSSLGTKTYT CN
VDHKPSNTKVDKRVESKYGPPCPSCPAPEFLGGPSVFLFPPKPKDTLMIS
RTPEVTCVVVDVS QEDPEVQFNWYVDGVEVHNAKTKPREEQFNSTYRVV
SVLTVLHQDWLNGKEYKCKVSNKGLPSSI EKTISKAKGQPREPQVYTLP
PSQEEMTKNQVSLTCLVKGFYPSDIAVEWESNGQPENNYKTTPPV LDSD
GSFFLYSRLTVDKSRWQEGNVFSCSVMHEALHNHYTQKSLSLSLGKGGGG
AGGGGELQMTQ SPSSLSASVGDRVTITCRTSQSISSYLNWYQQKPGQPP
KLLIYWASTRESGVPDRFSGSGSGTDF TLTISSLQPEDSATYYCQQSYD
IPYTFGQGTKLEIKGGGGSGGGGSGGGGSEVQLLESGGGVVQP GRSLRL
SCAASGFTFSSYGMHWVRQAPGKGLEWVAVISYDGSNKYYADSVKGRFTI
SRDNSKNTL YLQMNSLRAEDTAVYYCAKDMGWGSGWRPYYYYGMDVWGQ
GTTVTVSS

### > Seq 174: Adecatumumab-Nivolumab (EpCAM-PD-1)IgG4-SPLE C-Terminal scFv Bivalent Bispecific PD1-sAg scFv Orientation 2

> Light Chain:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 175: Adecatumumab-Nivolumab IgG1-KIH-LALA C-Terminal scFv PD1 Monovalent, sAg Bivalent Bispecific 1

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GKGGGGAGGGGQVQLVESGGGVVQPGRSLRLDCKASGI
TFSNSGMHWVRQAPGKGLEWVAVIWYD GSKRYYADSVKGRFTISRDNSK
NTLFLQMNSLRAEDTAVYYCATNDDYWGQGTLVTVSSGGGGSG GGGSGG
GGSEIVLTQSPATLSLSPGERATLSCRASQSVSSYLAWYQQKPGQAPRLL
IYDASNRAT GIPARFSGSGSGTDFTLTISSLEPEDFAVYYCQQSSNWPR
TFGQGTKVEIK

### > Seq 176: Adecatumumab-Nivolumab IgG1-KIH-LALA C-Terminal scFv PD1 Monovalent, sAg Bivalent Bispecific 1

> Heavy Chain 2:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 177: Adecatumumab-Nivolumab (EpCAM-PD-1)IgG1-KIH-LALA C-Terminal scFv PD1 Monovalent, sAg Bivalent Bispecific 1

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 178: Adecatumumab-Nivolumab IgG1-KIH-LALA C-Terminal scFv PD1 Monovalent, sAg Bivalent Bispecific 2

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GKGGGGAGGGGEIVLTQSPATLSLSPGERATLSCRASQ
SVSSYLAWYQQKPGQAPRLLIYDASNR ATGIPARFSGSGSGTDFTLTIS
SLEPEDFAVYYCQQSSNWPRTFGQGTKVEIKGGGGSGGGGSGG GGSQVQ
LVESGGGVVQPGRSLRLDCKASGITFSNSGMHWVRQAPGKGLEWVAVIWY
DGSKRYYAD SVKGRFTISRDNSKNTLFLQMNSLRAEDTAVYYCATNDDY
WGQGTLVTVSS

### > Seq 178: Adecatumumab-Nivolumab IgG1-KIH-LALA C-Terminal scFv PD1 Monovalent, sAg Bivalent Bispecific 2

> Heavy Chain 2:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 179: Adecatumumab-Nivolumab (EpCAM-PD-1)IgG1-KIH-LALA C-Terminal scFv PD1 Monovalent, sAg Bivalent Bispecific 2

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 180: Adecatumumab-Nivolumab IgG1-KIH-LALA C-Terminal scFv PD1 Monovalent, sAg Bivalent Bispecific 3

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GKGGGGAGGGGQVQLVESGGGVVQPGRSLRLDCKASGI
TFSNSGMHWVRQAPGKGLEWVAVIWYD GSKRYYADSVKGRFTISRDNSK
NTLFLQMNSLRAEDTAVYYCATNDDYWGQGTLVTVSSGGGGSG GGGSGG
GGSEIVLTQSPATLSLSPGERATLSCRASQSVSSYLAWYQQKPGQAPRLL
IYDASNRAT GIPARFSGSGSGTDFTLTISSLEPEDFAVYYCQQSSNWPR
TFGQGTKVEIK

### > Seq 181: Adecatumumab-Nivolumab IgG1-KIH-LALA C-Terminal scFv PD1 Monovalent, sAg Bivalent Bispecific 3

> Heavy Chain 2:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 182: Adecatumumab-Nivolumab (EpCAM-PD-1)IgG1-KIH-LALA C-Terminal scFv PD1 Monovalent, sAg Bivalent Bispecific 3

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 183: Adecatumumab-Nivolumab IgG1-KIH-LALA C-Terminal scFv PD1 Monovalent, sAg Bivalent Bispecific 4

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GKGGGGAGGGGEIVLTQSPATLSLSPGERATLSCRASQ
SVSSYLAWYQQKPGQAPRLLIYDASNR ATGIPARFSGSGSGTDFTLTIS
SLEPEDFAVYYCQQSSNWPRTFGQGTKVEIKGGGGSGGGGSGG GGSQVQ
LVESGGGVVQPGRSLRLDCKASGITFSNSGMHWVRQAPGKGLEWVAVIWY
DGSKRYYAD SVKGRFTISRDNSKNTLFLQMNSLRAEDTAVYYCATNDDY
WGQGTLVTVSS

### > Seq 184: Adecatumumab-Nivolumab (EpCAM-PD-1)IgG1-KIH-LALA C-Terminal scFv PD1 Monovalent, sAg Bivalent Bispecific 4

> Heavy Chain 2:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 185: Adecatumumab-Nivolumab (EpCAM-PD-1)IgG1-KIH-LALA C-Terminal scFv PD1 Monovalent, sAg Bivalent Bispecific 4

> Light Chain:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 186: Adecatumumab-Nivolumab IgG1-KIH-LALA Monovalent Bispecific 1

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 187: Adecatumumab-Nivolumab (EpCAM-PD-1)IgG1-KIH-LALA Monovalent Bispecific 1

> Heavy Chain 2:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPSSKS TSGGTAALGCLVKDYFPE
PVTVSWNSGALTSGVHTFPAVLQSSGLYSLSSVVTVPSSSLGTQTYI CN
VNHKPSNTKVDKKVEPKSCDKTHTCPPCPAPEAAGGPSVFLFPPKPKDTL
MISRTPEVTCVVV DVSHEDPEVKFNWYVDGVEVHNAKTKPREEQYNSTY
RVVSVLTVLHQDWLNGKEYKCKVSNKALP APIEKTISKAKGQPREPQVY
TLPPSRDELTKNQVSLWCLVKGFYPSDIAVEWESNGQPENNYKTT PPVL
DSDGSFFLYSKLTVDKSRWQQGNVFSCSVMHEALHNHYTQKSLSLSPGK

### > Seq 188: Adecatumumab-Nivolumab (EpCAM-PD-1)IgG1-KIH-LALA Monovalent Bispecific 1

> Light Chain 1:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 189: Adecatumumab-Nivolumab (EpCAM-PD-1)IgG1-KIH-LALA Monovalent Bispecific 1

> Light Chain 2:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 190: Adecatumumab-Nivolumab IgG1-KIH-LALA Monovalent Bispecific 2

> Heavy Chain 1:

EVQLLESGGGVVQPGRSLRLSCAASGFTFSSYGMHWVRQAPGKGLEWVAV
ISYDGSNKYYADSVK GRFTISRDNSKNTLYLQMNSLRAEDTAVYYCAKD
MGWGSGWRPYYYYGMDVWGQGTTVTVSSAST KGPSVFPLAPSSKSTSGG
TAALGCLVKDYFPEPVTVSWNSGALTSGVHTFPAVLQSSGLYSLSSV VT
VPSSSLGTQTYICNVNHKPSNTKVDKKVEPKSCDKTHTCPPCPAPEAAGG
PSVFLFPPKPKDT LMISRTPEVTCVVVDVSHEDPEVKFNWYVDGVEVHN
AKTKPREEQYNSTYRVVSVLTVLHQDWLN GKEYKCKVSNKALPAPIEKT
ISKAKGQPREPQVYTLPPSRDELTKNQVSLWCLVKGFYPSDIAVE WESN
GQPENNYKTTPPVLDSDGSFFLYSKLTVDKSRWQQGNVFSCSVMHEALHN
HYTQKSLSLSP GK

### > Seq 191: Adecatumumab-Nivolumab (EpCAM-PD-1)IgG1-KIH-LALA Monovalent Bispecific 2

> Heavy Chain 2:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPSSKS TSGGTAALGCLVKDYFPE
PVTVSWNSGALTSGVHTFPAVLQSSGLYSLSSVVTVPSSSLGTQTYI CN
VNHKPSNTKVDKKVEPKSCDKTHTCPPCPAPEAAGGPSVFLFPPKPKDTL
MISRTPEVTCVVV DVSHEDPEVKFNWYVDGVEVHNAKTKPREEQYNSTY
RVVSVLTVLHQDWLNGKEYKCKVSNKALP APIEKTISKAKGQPREPQVY
TLPPSRDELTKNQVSLWCLVKGFYPSDIAVEWESNGQPENNYKTT PPVL
DSDGSFFLYSKLTVDKSRWQQGNVFSCSVMHEALHNHYTQKSLSLSPGK

### > Seq 192: Adecatumumab-Nivolumab (EpCAM-PD-1)IgG1-KIH-LALA Monovalent Bispecific 2

> Light Chain 1:

ELQMTQSPSSLSASVGDRVTITCRTSQSISSYLNWYQQKPGQPPKLLIYW
ASTRESGVPDRFSGS GSGTDFTLTISSLQPEDSATYYCQQSYDIPYTFG
QGTKLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 193: Adecatumumab-Nivolumab (EpCAM-PD-1)IgG1-KIH-LALA Monovalent Bispecific 2

> Light Chain 2:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 194: BB7.2-Pembrolizumab (HLA-A2-PD-1)IgG4-SPLE C-Terminal scFv Bivalent Bispecific sAg-PD-1 scFv Orientation 1

> Heavy Chain:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PCSRSTSESTAALGCLVK
DYFPEPVTVSWNSGALTSGVHTFPAVLQSSGLYSLSSVVTVPSSSLG TK
TYTCNVDHKPSNTKVDKRVESKYGPPCPSCPAPEFLGGPSVFLFPPKPKD
TLMISRTPEVTCV VVDVSQEDPEVQFNWYVDGVEVHNAKTKPREEQFNS
TYRVVSVLTVLHQDWLNGKEYKCKVSNKG LPSSIEKTISKAKGQPREPQ
VYTLPPSQEEMTKNQVSLTCLVKGFYPSDIAVEWESNGQPENNYK TTPP
VLDSDGSFFLYSRLTVDKSRWQEGNVFSCSVMHEALHNHYTQKSLSLSLG
KGGGGAGGGGQ VQLVQSGVEVKKPGASVKVSCKASGYTFTNYYMYWVRQ
APGQGLEWMGGINPSNGGTNFNEKFKN RVTLTTDSSTTTAYMELKSLQF
DDTAVYYCARRDYRFDMGFDYWGQGTTVTVSSGGGGSGGGGSG GGGSEI
VLTQSPATLSLSPGERATLSCRASKGVSTSGYSYLHWYQQKPGQAPRLLI
YLASYLESG VPARFSGSGSGTDFTLTISSLEPEDFAVYYCQHSRDLPLT
FGGGTKVEIK

### > Seq 195: BB7.2-Pembrolizumab (HLA-A2-PD-1)IgG4-SPLE C-Terminal scFv Bivalent Bispecific sAg-PD-1 scFv Orientation 1

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 196: BB7.2-Pembrolizumab IgG4-SPLE C-Terminal scFv Bivalent Bispecific sAg-PD-1 scFv Orientation 2

> Heavy Chain:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PCSRSTSESTAALGCLVK
DYFPEPVTVSWNSGALTSGVHTFPAVLQSSGLYSLSSVVTVPSSSLG TK
TYTCNVDHKPSNTKVDKRVESKYGPPCPSCPAPEFLGGPSVFLFPPKPKD
TLMISRTPEVTCV VVDVSQEDPEVQFNWYVDGVEVHNAKTKPREEQFNS
TYRVVSVLTVLHQDWLNGKEYKCKVSNKG LPSSIEKTISKAKGQPREPQ
VYTLPPSQEEMTKNQVSLTCLVKGFYPSDIAVEWESNGQPENNYK TTPP
VLDSDGSFFLYSRLTVDKSRWQEGNVFSCSVMHEALHNHYTQKSLSLSLG
KGGGGAGGGGE IVLTQSPATLSLSPGERATLSCRASKGVSTSGYSYLHW
YQQKPGQAPRLLIYLASYLESGVPARF SGSGSGTDFTLTISSLEPEDFA
VYYCQHSRDLPLTFGGGTKVEIKGGGGSGGGGSGGGGSQVQLV QSGVEV
KKPGASVKVSCKASGYTFTNYYMYWVRQAPGQGLEWMGGINPSNGGTNFN
EKFKNRVTL TTDSSTTTAYMELKSLQFDDTAVYYCARRDYRFDMGFDYW
GQGTTVTVSS

### > Seq 197: BB7.2-Pembrolizumab (HLA-A2-PD-1)IgG4-SPLE C-Terminal scFv Bivalent Bispecific sAg-PD-1 scFv Orientation 2

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 198: BB7.2-Pembrolizumab IgG4-SPLE C-Terminal scFv Bivalent Bispecific PD-1-sAg scFv Orientation 1

> Heavy Chain:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPCSRSTSESTAALGCL
VKDYFPEPVTVSWNSGALTSGVHTFPAVLQSSGLYSLSSVVTVPSSS LG
TKTYTCNVDHKPSNTKVDKRVESKYGPPCPSCPAPEFLGGPSVFLFPPKP
KDTLMISRTPEVT CVVVDVSQEDPEVQFNWYVDGVEVHNAKTKPREEQF
NSTYRVVSVLTVLHQDWLNGKEYKCKVSN KGLPSSIEKTISKAKGQPRE
PQVYTLPPSQEEMTKNQVSLTCLVKGFYPSDIAVEWESNGQPENN YKTT
PPVLDSDGSFFLYSRLTVDKSRWQEGNVFSCSVMHEALHNHYTQKSLSLS
LGKGGGGAGGG GQVQLQQSGPELVKPGASVKMSCKASGYTFTSYHIQWV
KQRPGQGLEWIGWIYPGDGSTQYNEKF KGKTTLTADKSSSTAYMLLSSL
TSEDSAIYFCAREGTYYAMDYWGQGTSVTVSSGGGGSGGGGSG GGGSDV
LMTQTPLSLPVSLGDQVSISCRSSQSIVHSNGNTYLEWYLQKPGQSPKLL
IYKVSNRFS GVPDRFSGSGSGTDFTLKISRVEAEDLGVYYCFQGSHVPR
TFGGGTKLEIK

### > Seq 199: BB7.2-Pembrolizumab (HLA-A2-PD-1)IgG4-SPLE C-Terminal scFv Bivalent Bispecific PD-1-sAg scFv Orientation 1

> Light Chain:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 200: BB7.2-Pembrolizumab IgG4-SPLE C-Terminal scFv Bivalent Bispecific PD1-sAg scFv Orientation 2

> Heavy Chain:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPCSRSTSESTAALGCL
VKDYFPEPVTVSWNSGALTSGVHTFPAVLQSSGLYSLSSVVTVPSSS LG
TKTYTCNVDHKPSNTKVDKRVESKYGPPCPSCPAPEFLGGPSVFLFPPKP
KDTLMISRTPEVT CVVVDVSQEDPEVQFNWYVDGVEVHNAKTKPREEQF
NSTYRVVSVLTVLHQDWLNGKEYKCKVSN KGLPSSIEKTISKAKGQPRE
PQVYTLPPSQEEMTKNQVSLTCLVKGFYPSDIAVEWESNGQPENN YKTT
PPVLDSDGSFFLYSRLTVDKSRWQEGNVFSCSVMHEALHNHYTQKSLSLS
LGKGGGGAGGG GDVLMTQTPLSLPVSLGDQVSISCRSSQSIVHSNGNTY
LEWYLQKPGQSPKLLIYKVSNRFSGVP DRFSGSGSGTDFTLKISRVEAE
DLGVYYCFQGSHVPRTFGGGTKLEIKGGGGSGGGGSGGGGSQV QLQQSG
PELVKPGASVKMSCKASGYTFTSYHIQWVKQRPGQGLEWIGWIYPGDGST
QYNEKFKGK TTLTADKSSSTAYMLLSSLTSEDSAIYFCAREGTYYAMDY
WGQGTSVTVSS

### > Seq 201: BB7.2-Pembrolizumab (HLA-A2-PD-1)IgG4-SPLE C-Terminal scFv Bivalent Bispecific PD1-sAg scFv Orientation 2

> Light Chain:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 202: BB7.2-Pembrolizumab IgG1-KIH-LALA C-Terminal scFv PD1 Monovalent, sAg Bivalent Bispecific 1

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGKGGGGAGG GGQVQLVQSGVEVKKPGASVKVSCKASGYTFTNYYMYW
VRQAPGQGLEWMGGINPSNGGTNFNEK FKNRVTLTTDSSTTTAYMELKS
LQFDDTAVYYCARRDYRFDMGFDYWGQGTTVTVSSGGGGSGGG GSGGGG
SEIVLTQSPATLSLSPGERATLSCRASKGVSTSGYSYLHWYQQKPGQAPR
LLIYLASYL ESGVPARFSGSGSGTDFTLTISSLEPEDFAVYYCQHSRDL
PLTFGGGTKVEIK

### > Seq 203: BB7.2-Pembrolizumab IgG1-KIH-LALA C-Terminal scFv PD1 Monovalent, sAg Bivalent Bispecific 1

> Heavy Chain 2:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 204: BB7.2-Pembrolizumab (HLA-A2-PD-1)IgG1-KIH-LALA C-Terminal scFv PD1 Monovalent, sAg Bivalent Bispecific 1

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 205: BB7.2-Pembrolizumab IgG1-KIH-LALA C-Terminal scFv PD1 Monovalent, sAg Bivalent Bispecific 2

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGKGGGGAGG GGEIVLTQSPATLSLSPGERATLSCRASKGVSTSGYSY
LHWYQQKPGQAPRLLIYLASYLESGVP ARFSGSGSGTDFTLTISSLEPE
DFAVYYCQHSRDLPLTFGGGTKVEIKGGGGSGGGGSGGGGSQV QLVQSG
VEVKKPGASVKVSCKASGYTFTNYYMYWVRQAPGQGLEWMGGINPSNGGT
NFNEKFKNR VTLTTDSSTTTAYMELKSLQFDDTAVYYCARRDYRFDMGF
DYWGQGTTVTVSS

### > Seq 205: BB7.2-Pembrolizumab IgG1-KIH-LALA C-Terminal scFv PD1 Monovalent, sAg Bivalent Bispecific 2

> Heavy Chain 2:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 206: BB7.2-Pembrolizumab (HLA-A2-PD-1)IgG1-KIH-LALA C-Terminal scFv PD1 Monovalent, sAg Bivalent Bispecific 2

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 207: BB7.2-Pembrolizumab IgG1-KIH-LALA C-Terminal scFv PD1 Monovalent, sAg Bivalent Bispecific 3

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGKGGGGAGG GGQVQLVQSGVEVKKPGASVKVSCKASGYTFTNYYMYW
VRQAPGQGLEWMGGINPSNGGTNFNEK FKNRVTLTTDSSTTTAYMELKS
LQFDDTAVYYCARRDYRFDMGFDYWGQGTTVTVSSGGGGSGGG GSGGGG
SEIVLTQSPATLSLSPGERATLSCRASKGVSTSGYSYLHWYQQKPGQAPR
LLIYLASYL ESGVPARFSGSGSGTDFTLTISSLEPEDFAVYYCQHSRDL
PLTFGGGTKVEIK

### > Seq 208: BB7.2-Pembrolizumab IgG1-KIH-LALA C-Terminal scFv PD1 Monovalent, sAg Bivalent Bispecific 3

> Heavy Chain 2:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 209: BB7.2-Pembrolizumab (HLA-A2-PD-1)IgG1-KIH-LALA C-Terminal scFv PD1 Monovalent, sAg Bivalent Bispecific 3

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 210: BB7.2-Pembrolizumab IgG1-KIH-LALA C-Terminal scFv PD1 Monovalent, sAg Bivalent Bispecific 4

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGKGGGGAGG GGEIVLTQSPATLSLSPGERATLSCRASKGVSTSGYSY
LHWYQQKPGQAPRLLIYLASYLESGVP ARFSGSGSGTDFTLTISSLEPE
DFAVYYCQHSRDLPLTFGGGTKVEIKGGGGSGGGGSGGGGSQV QLVQSG
VEVKKPGASVKVSCKASGYTFTNYYMYWVRQAPGQGLEWMGGINPSNGGT
NFNEKFKNR VTLTTDSSTTTAYMELKSLQFDDTAVYYCARRDYRFDMGF
DYWGQGTTVTVSS

### > Seq 211: BB7.2-Pembrolizumab (HLA-A2-PD-1)IgG1-KIH-LALA C-Terminal scFv PD1 Monovalent, sAg Bivalent Bispecific 4

> Heavy Chain 2:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 212: BB7.2-Pembrolizumab (HLA-A2-PD-1)IgG1-KIH-LALA C-Terminal scFv PD1 Monovalent, sAg Bivalent Bispecific 4

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 213: BB7.2-Pembrolizumab IgG1-KIH-LALA Monovalent Bispecific 1

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 214: BB7.2-Pembrolizumab (HLA-A2-PD-1)IgG1-KIH-LALA Monovalent Bispecific 1

> Heavy Chain 2:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPSSKSTSGGTAALGCL
VKDYFPEPVTVSWNSGALTSGVHTFPAVLQSSGLYSLSSVVTVPSSS LG
TQTYICNVNHKPSNTKVDKKVEPKSCDKTHTCPPCPAPEAAGGPSVFLFP
PKPKDTLMISRTP EVTCVVVDVSHEDPEVKFNWYVDGVEVHNAKTKPRE
EQYNSTYRVVSVLTVLHQDWLNGKEYKCK VSNKALPAPIEKTISKAKGQ
PREPQVYTLPPSRDELTKNQVSLWCLVKGFYPSDIAVEWESNGQP ENNY
KTTPPVLDSDGSFFLYSKLTVDKSRWQQGNVFSCSVMHEALHNHYTQKSL
SLSPGK

### > Seq 215: BB7.2-Pembrolizumab (HLA-A2-PD-1)IgG1-KIH-LALA Monovalent Bispecific 1

> Light Chain 1:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 216: BB7.2-Pembrolizumab (HLA-A2-PD-1)IgG1-KIH-LALA Monovalent Bispecific 1

> Light Chain 2:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 217: BB7.2-Pembrolizumab IgG1-KIH-LALA Monovalent Bispecific 2

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 218: BB7.2-Pembrolizumab (HLA-A2-PD-1)IgG1-KIH-LALA Monovalent Bispecific 2

> Heavy Chain 2:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPSSKSTSGGTAALGCL
VKDYFPEPVTVSWNSGALTSGVHTFPAVLQSSGLYSLSSVVTVPSSS LG
TQTYICNVNHKPSNTKVDKKVEPKSCDKTHTCPPCPAPEAAGGPSVFLFP
PKPKDTLMISRTP EVTCVVVDVSHEDPEVKFNWYVDGVEVHNAKTKPRE
EQYNSTYRVVSVLTVLHQDWLNGKEYKCK VSNKALPAPIEKTISKAKGQ
PREPQVYTLPPSRDELTKNQVSLWCLVKGFYPSDIAVEWESNGQP ENNY
KTTPPVLDSDGSFFLYSKLTVDKSRWQQGNVFSCSVMHEALHNHYTQKSL
SLSPGK

### > Seq 219: BB7.2-Pembrolizumab (HLA-A2-PD-1)IgG1-KIH-LALA Monovalent Bispecific 2

> Light Chain 1:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 220: BB7.2-Pembrolizumab (HLA-A2-PD-1)IgG1-KIH-LALA Monovalent Bispecific 2

> Light Chain 2:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 221: BB7.2-Nivolumab (HLA-A2-PD-1)IgG4-SPLE C-Terminal scFv Bivalent Bispecific sAg-PD-1 scFv Orientation 1

> Heavy Chain:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PCSRSTSESTAALGCLVK
DYFPEPVTVSWNSGALTSGVHTFPAVLQSSGLYSLSSVVTVPSSSLG TK
TYTCNVDHKPSNTKVDKRVESKYGPPCPSCPAPEFLGGPSVFLFPPKPKD
TLMISRTPEVTCV VVDVSQEDPEVQFNWYVDGVEVHNAKTKPREEQFNS
TYRVVSVLTVLHQDWLNGKEYKCKVSNKG LPSSIEKTISKAKGQPREPQ
VYTLPPSQEEMTKNQVSLTCLVKGFYPSDIAVEWESNGQPENNYK TTPP
VLDSDGSFFLYSRLTVDKSRWQEGNVFSCSVMHEALHNHYTQKSLSLSLG
KGGGGAGGGGQ VQLVESGGGVVQPGRSLRLDCKASGITFSNSGMHWVRQ
APGKGLEWVAVIWYDGSKRYYADSVKG RFTISRDNSKNTLFLQMNSLRA
EDTAVYYCATNDDYWGQGTLVTVSSGGGGSGGGGSGGGGSEIV LTQSPA
TLSLSPGERATLSCRASQSVSSYLAWYQQKPGQAPRLLIYDASNRATGIP
ARFSGSGSG TDFTLTISSLEPEDFAVYYCQQSSNWPRTFGQGTKVEIK

### > Seq 222: BB7.2-Nivolumab (HLA-A2-PD-1)IgG4-SPLE C-Terminal scFv Bivalent Bispecific sAg-PD-1 scFv Orientation 1

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 223: BB7.2-Nivolumab IgG4-SPLE C-Terminal scFv Bivalent Bispecific sAg-PD-1 scFv Orientation 2

> Heavy Chain:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PCSRSTSESTAALGCLVK
DYFPEPVTVSWNSGALTSGVHTFPAVLQSSGLYSLSSVVTVPSSSLG TK
TYTCNVDHKPSNTKVDKRVESKYGPPCPSCPAPEFLGGPSVFLFPPKPKD
TLMISRTPEVTCV VVDVSQEDPEVQFNWYVDGVEVHNAKTKPREEQFNS
TYRVVSVLTVLHQDWLNGKEYKCKVSNKG LPSSIEKTISKAKGQPREPQ
VYTLPPSQEEMTKNQVSLTCLVKGFYPSDIAVEWESNGQPENNYK TTPP
VLDSDGSFFLYSRLTVDKSRWQEGNVFSCSVMHEALHNHYTQKSLSLSLG
KGGGGAGGGGE IVLTQSPATLSLSPGERATLSCRASQSVSSYLAWYQQK
PGQAPRLLIYDASNRATGIPARFSGSG SGTDFTLTISSLEPEDFAVYYC
QQSSNWPRTFGQGTKVEIKGGGGSGGGGSGGGGSQVQLVESGG GVVQPG
RSLRLDCKASGITFSNSGMHWVRQAPGKGLEWVAVIWYDGSKRYYADSVK
GRFTISRDN SKNTLFLQMNSLRAEDTAVYYCATNDDYWGQGTLVTVSS

### > Seq 224: BB7.2-Nivolumab (HLA-A2-PD-1)IgG4-SPLE C-Terminal scFv Bivalent Bispecific sAg-PD-1 scFv Orientation 2

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 225: BB7.2-Nivolumab IgG4-SPLE C-Terminal scFv Bivalent Bispecific PD-1-sAg scFv Orientation 1

> Heavy Chain:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPCSRS TSESTAALGCLVKDYFPE
PVTVSWNSGALTSGVHTFPAVLQSSGLYSLSSVVTVPSSSLGTKTYT CN
VDHKPSNTKVDKRVESKYGPPCPSCPAPEFLGGPSVFLFPPKPKDTLMIS
RTPEVTCVVVDVS QEDPEVQFNWYVDGVEVHNAKTKPREEQFNSTYRVV
SVLTVLHQDWLNGKEYKCKVSNKGLPSSI EKTISKAKGQPREPQVYTLP
PSQEEMTKNQVSLTCLVKGFYPSDIAVEWESNGQPENNYKTTPPV LDSD
GSFFLYSRLTVDKSRWQEGNVFSCSVMHEALHNHYTQKSLSLSLGKGGGG
AGGGGQVQLQQ SGPELVKPGASVKMSCKASGYTFTSYHIQWVKQRPGQG
LEWIGWIYPGDGSTQYNEKFKGKTTLT ADKSSSTAYMLLSSLTSEDSAI
YFCAREGTYYAMDYWGQGTSVTVSSGGGGSGGGGSGGGGSDVL MTQTPL
SLPVSLGDQVSISCRSSQSIVHSNGNTYLEWYLQKPGQSPKLLIYKVSNR
FSGVPDRFS GSGSGTDFTLKISRVEAEDLGVYYCFQGSHVPRTFGGGTK
LEIK

### > Seq 226: BB7.2-Nivolumab (HLA-A2-PD-1)IgG4-SPLE C-Terminal scFv Bivalent Bispecific PD-1-sAg scFv Orientation 1

> Light Chain:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 227: BB7.2-Nivolumab IgG4-SPLE C-Terminal scFv Bivalent Bispecific PD1-sAg scFv Orientation 2

> Heavy Chain:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPCSRS TSESTAALGCLVKDYFPE
PVTVSWNSGALTSGVHTFPAVLQSSGLYSLSSVVTVPSSSLGTKTYT CN
VDHKPSNTKVDKRVESKYGPPCPSCPAPEFLGGPSVFLFPPKPKDTLMIS
RTPEVTCVVVDVS QEDPEVQFNWYVDGVEVHNAKTKPREEQFNSTYRVV
SVLTVLHQDWLNGKEYKCKVSNKGLPSSI EKTISKAKGQPREPQVYTLP
PSQEEMTKNQVSLTCLVKGFYPSDIAVEWESNGQPENNYKTTPPV LDSD
GSFFLYSRLTVDKSRWQEGNVFSCSVMHEALHNHYTQKSLSLSLGKGGGG
AGGGGDVLMTQ TPLSLPVSLGDQVSISCRSSQSIVHSNGNTYLEWYLQK
PGQSPKLLIYKVSNRFSGVPDRFSGSG SGTDFTLKISRVEAEDLGVYYC
FQGSHVPRTFGGGTKLEIKGGGGSGGGGSGGGGSQVQLQQSGP ELVKPG
ASVKMSCKASGYTFTSYHIQWVKQRPGQGLEWIGWIYPGDGSTQYNEKFK
GKTTLTADK SSSTAYMLLSSLTSEDSAIYFCAREGTYYAMDYWGQGTSV
TVSS

### > Seq 228: BB7.2-Nivolumab (HLA-A2-PD-1)IgG4-SPLE C-Terminal scFv Bivalent Bispecific PD1-sAg scFv Orientation 2

> Light Chain:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 229: BB7.2-Nivolumab IgG1-KIH-LALA C-Terminal scFv PD1 Monovalent, sAg Bivalent Bispecific 1

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGKGGGGAGG GGQVQLVESGGGVVQPGRSLRLDCKASGITFSNSGMHW
VRQAPGKGLEWVAVIWYDGSKRYYADS VKGRFTISRDNSKNTLFLQMNS
LRAEDTAVYYCATNDDYWGQGTLVTVSSGGGGSGGGGSGGGGS EIVLTQ
SPATLSLSPGERATLSCRASQSVSSYLAWYQQKPGQAPRLLIYDASNRAT
GIPARFSGS GSGTDFTLTISSLEPEDFAVYYCQQSSNWPRTFGQGTKVE
IK

### > Seq 230: BB7.2-Nivolumab IgG1-KIH-LALA C-Terminal scFv PD1 Monovalent, sAg Bivalent Bispecific 1

> Heavy Chain 2:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 231: BB7.2-Nivolumab (HLA-A2-PD-1)IgG1-KIH-LALA C-Terminal scFv PD1 Monovalent, sAg Bivalent Bispecific 1

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 232: BB7.2-Nivolumab IgG1-KIH-LALA C-Terminal scFv PD1 Monovalent, sAg Bivalent Bispecific 2

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGKGGGGAGG GGEIVLTQSPATLSLSPGERATLSCRASQSVSSYLAWY
QQKPGQAPRLLIYDASNRATGIPARFS GSGSGTDFTLTISSLEPEDFAV
YYCQQSSNWPRTFGQGTKVEIKGGGGSGGGGSGGGGSQVQLVE SGGGVV
QPGRSLRLDCKASGITFSNSGMHWVRQAPGKGLEWVAVIWYDGSKRYYAD
SVKGRFTIS RDNSKNTLFLQMNSLRAEDTAVYYCATNDDYWGQGTLVTV
SS

### > Seq 232: BB7.2-Nivolumab IgG1-KIH-LALA C-Terminal scFv PD1 Monovalent, sAg Bivalent Bispecific 2

> Heavy Chain 2:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 233: BB7.2-Nivolumab (HLA-A2-PD-1)IgG1-KIH-LALA C-Terminal scFv PD1 Monovalent, sAg Bivalent Bispecific 2

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 234: BB7.2-Nivolumab IgG1-KIH-LALA C-Terminal scFv PD1 Monovalent, sAg Bivalent Bispecific 3

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGKGGGGAGG GGQVQLVESGGGVVQPGRSLRLDCKASGITFSNSGMHW
VRQAPGKGLEWVAVIWYDGSKRYYADS VKGRFTISRDNSKNTLFLQMNS
LRAEDTAVYYCATNDDYWGQGTLVTVSSGGGGSGGGGSGGGGS EIVLTQ
SPATLSLSPGERATLSCRASQSVSSYLAWYQQKPGQAPRLLIYDASNRAT
GIPARFSGS GSGTDFTLTISSLEPEDFAVYYCQQSSNWPRTFGQGTKVE
IK

### > Seq 235: BB7.2-Nivolumab IgG1-KIH-LALA C-Terminal scFv PD1 Monovalent, sAg Bivalent Bispecific 3

> Heavy Chain 2:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 236: BB7.2-Nivolumab (HLA-A2-PD-1)IgG1-KIH-LALA C-Terminal scFv PD1 Monovalent, sAg Bivalent Bispecific 3

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 237: BB7.2-Nivolumab IgG1-KIH-LALA C-Terminal scFv PD1 Monovalent, sAg Bivalent Bispecific 4

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGKGGGGAGG GGEIVLTQSPATLSLSPGERATLSCRASQSVSSYLAWY
QQKPGQAPRLLIYDASNRATGIPARFS GSGSGTDFTLTISSLEPEDFAV
YYCQQSSNWPRTFGQGTKVEIKGGGGSGGGGSGGGGSQVQLVE SGGGVV
QPGRSLRLDCKASGITFSNSGMHWVRQAPGKGLEWVAVIWYDGSKRYYAD
SVKGRFTIS RDNSKNTLFLQMNSLRAEDTAVYYCATNDDYWGQGTLVTV
SS

### > Seq 238: BB7.2-Nivolumab (HLA-A2-PD-1)IgG1-KIH-LALA C-Terminal scFv PD1 Monovalent, sAg Bivalent Bispecific 4

> Heavy Chain 2:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 239: BB7.2-Nivolumab (HLA-A2-PD-1)IgG1-KIH-LALA C-Terminal scFv PD1 Monovalent, sAg Bivalent Bispecific 4

> Light Chain:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 240: BB7.2-Nivolumab IgG1-KIH-LALA Monovalent Bispecific 1

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 241: BB7.2-Nivolumab (HLA-A2-PD-1)IgG1-KIH-LALA Monovalent Bispecific 1

> Heavy Chain 2:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPSSKS TSGGTAALGCLVKDYFPE
PVTVSWNSGALTSGVHTFPAVLQSSGLYSLSSVVTVPSSSLGTQTYI CN
VNHKPSNTKVDKKVEPKSCDKTHTCPPCPAPEAAGGPSVFLFPPKPKDTL
MISRTPEVTCVVV DVSHEDPEVKFNWYVDGVEVHNAKTKPREEQYNSTY
RVVSVLTVLHQDWLNGKEYKCKVSNKALP APIEKTISKAKGQPREPQVY
TLPPSRDELTKNQVSLWCLVKGFYPSDIAVEWESNGQPENNYKTT PPVL
DSDGSFFLYSKLTVDKSRWQQGNVFSCSVMHEALHNHYTQKSLSLSPGK

### > Seq 242: BB7.2-Nivolumab (HLA-A2-PD-1)IgG1-KIH-LALA Monovalent Bispecific 1

> Light Chain 1:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 243: BB7.2-Nivolumab (HLA-A2-PD-1)IgG1-KIH-LALA Monovalent Bispecific 1

> Light Chain 2:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 244: BB7.2-Nivolumab IgG1-KIH-LALA Monovalent Bispecific 2

> Heavy Chain 1:

QVQLQQSGPELVKPGASVKMSCKASGYTFTSYHIQWVKQRPGQGLEWIGW
IYPGDGSTQYNEKFK GKTTLTADKSSSTAYMLLSSLTSEDSAIYFCARE
GTYYAMDYWGQGTSVTVSSASTKGPSVFPLA PSSKSTSGGTAALGCLVK
DYFPEPVTVSWNSGALTSGVHTFPAVLQSSGLYSLSSVVTVPSSSLG TQ
TYICNVNHKPSNTKVDKKVEPKSCDKTHTCPPCPAPEAAGGPSVFLFPPK
PKDTLMISRTPEV TCVVVDVSHEDPEVKFNWYVDGVEVHNAKTKPREEQ
YNSTYRVVSVLTVLHQDWLNGKEYKCKVS NKALPAPIEKTISKAKGQPR
EPQVYTLPPSRDELTKNQVSLWCLVKGFYPSDIAVEWESNGQPEN NYKT
TPPVLDSDGSFFLYSKLTVDKSRWQQGNVFSCSVMHEALHNHYTQKSLSL
SPGK

### > Seq 245: BB7.2-Nivolumab (HLA-A2-PD-1)IgG1-KIH-LALA Monovalent Bispecific 2

> Heavy Chain 2:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPSSKS TSGGTAALGCLVKDYFPE
PVTVSWNSGALTSGVHTFPAVLQSSGLYSLSSVVTVPSSSLGTQTYI CN
VNHKPSNTKVDKKVEPKSCDKTHTCPPCPAPEAAGGPSVFLFPPKPKDTL
MISRTPEVTCVVV DVSHEDPEVKFNWYVDGVEVHNAKTKPREEQYNSTY
RVVSVLTVLHQDWLNGKEYKCKVSNKALP APIEKTISKAKGQPREPQVY
TLPPSRDELTKNQVSLWCLVKGFYPSDIAVEWESNGQPENNYKTT PPVL
DSDGSFFLYSKLTVDKSRWQQGNVFSCSVMHEALHNHYTQKSLSLSPGK

### > Seq 246: BB7.2-Nivolumab (HLA-A2-PD-1)IgG1-KIH-LALA Monovalent Bispecific 2

> Light Chain 1:

DVLMTQTPLSLPVSLGDQVSISCRSSQSIVHSNGNTYLEWYLQKPGQSPK
LLIYKVSNRFSGVPD RFSGSGSGTDFTLKISRVEAEDLGVYYCFQGSHV
PRTFGGGTKLEIKRTVAAPSVFIFPPSDEQL KSGTASVVCLLNNFYPRE
AKVQWKVDNALQSGNSQESVTEQDSKDSTYSLSSTLTLSKADYEKHK VY
ACEVTHQGLSSPVTKSFNRGEC

### > Seq 247: BB7.2-Nivolumab (HLA-A2-PD-1)IgG1-KIH-LALA Monovalent Bispecific 2

> Light Chain 2:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 248: 9.49-Pembrolizumab (MHC II (Pan-Allele)-PD-1)IgG4-SPLE C-Terminal scFv Bivalent Bispecific sAg-PD-1 scFv Orientation 1

> Heavy Chain:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APCSRSTSESTAALGCLV
KDYFPEPVTVSWNSGALTSGVHTFPAVLQSSGLYSLSSVVTVPSSSL GT
KTYTCNVDHKPSNTKVDKRVESKYGPPCPSCPAPEFLGGPSVFLFPPKPK
DTLMISRTPEVTC VVVDVSQEDPEVQFNWYVDGVEVHNAKTKPREEQFN
STYRVVSVLTVLHQDWLNGKEYKCKVSNK GLPSSIEKTISKAKGQPREP
QVYTLPPSQEEMTKNQVSLTCLVKGFYPSDIAVEWESNGQPENNY KTTP
PVLDSDGSFFLYSRLTVDKSRWQEGNVFSCSVMHEALHNHYTQKSLSLSL
GKGGGGAGGGG QVQLVQSGVEVKKPGASVKVSCKASGYTFTNYYMYWVR
QAPGQGLEWMGGINPSNGGTNFNEKFK NRVTLTTDSSTTTAYMELKSLQ
FDDTAVYYCARRDYRFDMGFDYWGQGTTVTVSSGGGGSGGGGS GGGGSE
IVLTQSPATLSLSPGERATLSCRASKGVSTSGYSYLHWYQQKPGQAPRLL
IYLASYLES GVPARFSGSGSGTDFTLTISSLEPEDFAVYYCQHSRDLPL
TFGGGTKVEIK

### > Seq 249: 9.49-Pembrolizumab (MHC II (Pan-Allele)-PD-1)IgG4-SPLE C-Terminal scFv Bivalent Bispecific sAg-PD-1 scFv Orientation 1

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 250: 9.49-Pembrolizumab IgG4-SPLE C-Terminal scFv Bivalent Bispecific sAg-PD-1 scFv Orientation 2

> Heavy Chain:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APCSRSTSESTAALGCLV
KDYFPEPVTVSWNSGALTSGVHTFPAVLQSSGLYSLSSVVTVPSSSL GT
KTYTCNVDHKPSNTKVDKRVESKYGPPCPSCPAPEFLGGPSVFLFPPKPK
DTLMISRTPEVTC VVVDVSQEDPEVQFNWYVDGVEVHNAKTKPREEQFN
STYRVVSVLTVLHQDWLNGKEYKCKVSNK GLPSSIEKTISKAKGQPREP
QVYTLPPSQEEMTKNQVSLTCLVKGFYPSDIAVEWESNGQPENNY KTTP
PVLDSDGSFFLYSRLTVDKSRWQEGNVFSCSVMHEALHNHYTQKSLSLSL
GKGGGGAGGGG EIVLTQSPATLSLSPGERATLSCRASKGVSTSGYSYLH
WYQQKPGQAPRLLIYLASYLESGVPAR FSGSGSGTDFTLTISSLEPEDF
AVYYCQHSRDLPLTFGGGTKVEIKGGGGSGGGGSGGGGSQVQL VQSGVE
VKKPGASVKVSCKASGYTFTNYYMYWVRQAPGQGLEWMGGINPSNGGTNF
NEKFKNRVT LTTDSSTTTAYMELKSLQFDDTAVYYCARRDYRFDMGFDY
WGQGTTVTVSS

### > Seq 251: 9.49-Pembrolizumab (MHC II (Pan-Allele)-PD-1)IgG4-SPLE C-Terminal scFv Bivalent Bispecific sAg-PD-1 scFv Orientation 2

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 252: 9.49-Pembrolizumab IgG4-SPLE C-Terminal scFv Bivalent Bispecific PD-1-sAg scFv Orientation 1

> Heavy Chain:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPCSRSTSESTAALGCL
VKDYFPEPVTVSWNSGALTSGVHTFPAVLQSSGLYSLSSVVTVPSSS LG
TKTYTCNVDHKPSNTKVDKRVESKYGPPCPSCPAPEFLGGPSVFLFPPKP
KDTLMISRTPEVT CVVVDVSQEDPEVQFNWYVDGVEVHNAKTKPREEQF
NSTYRVVSVLTVLHQDWLNGKEYKCKVSN KGLPSSIEKTISKAKGQPRE
PQVYTLPPSQEEMTKNQVSLTCLVKGFYPSDIAVEWESNGQPENN YKTT
PPVLDSDGSFFLYSRLTVDKSRWQEGNVFSCSVMHEALHNHYTQKSLSLS
LGKGGGGAGGG GEVQLQESGPSLVRPSQTLSLTCSVTGDSITSSYWNWI
RKFPGNKLEYMGYITYSGNTYYTPSLK SRISITRDTSKNQYFLQLNSVT
TEDTATYYCSRDYGRGDYAMVYWGQGTSVTVSSGGGGSGGGGS GGGGSD
VVLTQSPATLSVTPGDSVSLSCRASQSIINNLHWYHQKSHESPRLLIKYA
SQSISGIPS RFSGSGSGTDFTLSINSVETEDFGVYFCQQSGSWPYTFGG
GSNLEIK

### > Seq 253: 9.49-Pembrolizumab (MHC II (Pan-Allele)-PD-1)IgG4-SPLE C-Terminal scFv Bivalent Bispecific PD-1-sAg scFv Orientation 1

> Light Chain:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 254: 9.49-Pembrolizumab IgG4-SPLE C-Terminal scFv Bivalent Bispecific PD1-sAg scFv Orientation 2

> Heavy Chain:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPCSRSTSESTAALGCL
VKDYFPEPVTVSWNSGALTSGVHTFPAVLQSSGLYSLSSVVTVPSSS LG
TKTYTCNVDHKPSNTKVDKRVESKYGPPCPSCPAPEFLGGPSVFLFPPKP
KDTLMISRTPEVT CVVVDVSQEDPEVQFNWYVDGVEVHNAKTKPREEQF
NSTYRVVSVLTVLHQDWLNGKEYKCKVSN KGLPSSIEKTISKAKGQPRE
PQVYTLPPSQEEMTKNQVSLTCLVKGFYPSDIAVEWESNGQPENN YKTT
PPVLDSDGSFFLYSRLTVDKSRWQEGNVFSCSVMHEALHNHYTQKSLSLS
LGKGGGGAGGG GDVVLTQSPATLSVTPGDSVSLSCRASQSIINNLHWYH
QKSHESPRLLIKYASQSISGIPSRFSG SGSGTDFTLSINSVETEDFGVY
FCQQSGSWPYTFGGGSNLEIKGGGGSGGGGSGGGGSEVQLQES GPSLVR
PSQTLSLTCSVTGDSITSSYWNWIRKFPGNKLEYMGYITYSGNTYYTPSL
KSRISITRD TSKNQYFLQLNSVTTEDTATYYCSRDYGRGDYAMVYWGQG
TSVTVSS

### > Seq 255: 9.49-Pembrolizumab (MHC II (Pan-Allele)-PD-1)IgG4-SPLE C-Terminal scFv Bivalent Bispecific PD1-sAg scFv Orientation 2

> Light Chain:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 256: 9.49-Pembrolizumab IgG1-KIH-LALA C-Terminal scFv PD1 Monovalent, sAg Bivalent Bispecific 1

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGKGGGGAG GGGQVQLVQSGVEVKKPGASVKVSCKASGYTFTNYYMY
WVRQAPGQGLEWMGGINPSNGGTNFNE KFKNRVTLTTDSSTTTAYMELK
SLQFDDTAVYYCARRDYRFDMGFDYWGQGTTVTVSSGGGGSGG GGSGGG
GSEIVLTQSPATLSLSPGERATLSCRASKGVSTSGYSYLHWYQQKPGQAP
RLLIYLASY LESGVPARFSGSGSGTDFTLTISSLEPEDFAVYYCQHSRD
LPLTFGGGTKVEIK

### > Seq 257: 9.49-Pembrolizumab IgG1-KIH-LALA C-Terminal scFv PD1 Monovalent, sAg Bivalent Bispecific 1

> Heavy Chain 2:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 258: 9.49-Pembrolizumab (MHC II (Pan-Allele)-PD-1)IgG1-KIH-LALA C-Terminal scFv PD1 Monovalent, sAg Bivalent Bispecific 1

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 259: 9.49-Pembrolizumab IgG1-KIH-LALA C-Terminal scFv PD1 Monovalent, sAg Bivalent Bispecific 2

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGKGGGGAG GGGEIVLTQSPATLSLSPGERATLSCRASKGVSTSGYS
YLHWYQQKPGQAPRLLIYLASYLESGV PARFSGSGSGTDFTLTISSLEP
EDFAVYYCQHSRDLPLTFGGGTKVEIKGGGGSGGGGSGGGGSQ VQLVQS
GVEVKKPGASVKVSCKASGYTFTNYYMYWVRQAPGQGLEWMGGINPSNGG
TNFNEKFKN RVTLTTDSSTTTAYMELKSLQFDDTAVYYCARRDYRFDMG
FDYWGQGTTVTVSS

### > Seq 259: 9.49-Pembrolizumab IgG1-KIH-LALA C-Terminal scFv PD1 Monovalent, sAg Bivalent Bispecific 2

> Heavy Chain 2:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 260: 9.49-Pembrolizumab (MHC II (Pan-Allele)-PD-1)IgG1-KIH-LALA C-Terminal scFv PD1 Monovalent, sAg Bivalent Bispecific 2

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 261: 9.49-Pembrolizumab IgG1-KIH-LALA C-Terminal scFv PD1 Monovalent, sAg Bivalent Bispecific 3

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGKGGGGAG GGGQVQLVQSGVEVKKPGASVKVSCKASGYTFTNYYMY
WVRQAPGQGLEWMGGINPSNGGTNFNE KFKNRVTLTTDSSTTTAYMELK
SLQFDDTAVYYCARRDYRFDMGFDYWGQGTTVTVSSGGGGSGG GGSGGG
GSEIVLTQSPATLSLSPGERATLSCRASKGVSTSGYSYLHWYQQKPGQAP
RLLIYLASY LESGVPARFSGSGSGTDFTLTISSLEPEDFAVYYCQHSRD
LPLTFGGGTKVEIK

### > Seq 262: 9.49-Pembrolizumab IgG1-KIH-LALA C-Terminal scFv PD1 Monovalent, sAg Bivalent Bispecific 3

> Heavy Chain 2:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 263: 9.49-Pembrolizumab (MHC II (Pan-Allele)-PD-1)IgG1-KIH-LALA C-Terminal scFv PD1 Monovalent, sAg Bivalent Bispecific 3

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 264: 9.49-Pembrolizumab IgG1-KIH-LALA C-Terminal scFv PD1 Monovalent, sAg Bivalent Bispecific 4

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGKGGGGAG GGGEIVLTQSPATLSLSPGERATLSCRASKGVSTSGYS
YLHWYQQKPGQAPRLLIYLASYLESGV PARFSGSGSGTDFTLTISSLEP
EDFAVYYCQHSRDLPLTFGGGTKVEIKGGGGSGGGGSGGGGSQ VQLVQS
GVEVKKPGASVKVSCKASGYTFTNYYMYWVRQAPGQGLEWMGGINPSNGG
TNFNEKFKN RVTLTTDSSTTTAYMELKSLQFDDTAVYYCARRDYRFDMG
FDYWGQGTTVTVSS

### > Seq 265: 9.49-Pembrolizumab (MHC II (Pan-Allele)-PD-1)IgG1-KIH-LALA C-Terminal scFv PD1 Monovalent, sAg Bivalent Bispecific 4

> Heavy Chain 2:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 266: 9.49-Pembrolizumab (MHC II (Pan-Allele)-PD-1)IgG1-KIH-LALA C-Terminal scFv PD1 Monovalent, sAg Bivalent Bispecific 4

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 267: 9.49-Pembrolizumab IgG1-KIH-LALA Monovalent Bispecific 1

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 268: 9.49-Pembrolizumab (MHC II (Pan-Allele)-PD-1)IgG1-KIH-LALA Monovalent Bispecific 1

> Heavy Chain 2:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPSSKSTSGGTAALGCL
VKDYFPEPVTVSWNSGALTSGVHTFPAVLQSSGLYSLSSVVTVPSSS LG
TQTYICNVNHKPSNTKVDKKVEPKSCDKTHTCPPCPAPEAAGGPSVFLFP
PKPKDTLMISRTP EVTCVVVDVSHEDPEVKFNWYVDGVEVHNAKTKPRE
EQYNSTYRVVSVLTVLHQDWLNGKEYKCK VSNKALPAPIEKTISKAKGQ
PREPQVYTLPPSRDELTKNQVSLWCLVKGFYPSDIAVEWESNGQP ENNY
KTTPPVLDSDGSFFLYSKLTVDKSRWQQGNVFSCSVMHEALHNHYTQKSL
SLSPGK

### > Seq 269: 9.49-Pembrolizumab (MHC II (Pan-Allele)-PD-1)IgG1-KIH-LALA Monovalent Bispecific 1

> Light Chain 1:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 270: 9.49-Pembrolizumab (MHC II (Pan-Allele)-PD-1)IgG1-KIH-LALA Monovalent Bispecific 1

> Light Chain 2:

EVLTQSPATLSLSPGERATLSCRASKGVSTSGYSYLHWYQQKPGQAPRLL
IYLASYLESGVPAR FSGSGSGTDFTLTISSLEPEDFAVYYCQHSRDLPL
TFGGGTKVEIKRTVAAPSVFIFPPSDEQLK SGTASVVCLLNNFYPREAK
VQWKVDNALQSGNSQESVTEQDSKDSTYSLSSTLTLSKADYEKHKV YAC
EVTHQGLSSPVTKSFNRGEC

### > Seq 271: 9.49-Pembrolizumab IgG1-KIH-LALA Monovalent Bispecific 2

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 272: 9.49-Pembrolizumab (MHC II (Pan-Allele)-PD-1)IgG1-KIH-LALA Monovalent Bispecific 2

> Heavy Chain 2:

QVQLVQSGVEVKKPGASVKVSCKASGYTFTNYYMYWVRQAPGQGLEWMGG
INPSNGGTNFNEKFK NRVTLTTDSSTTTAYMELKSLQFDDTAVYYCARR
DYRFDMGFDYWGQGTTVTVSSASTKGPSVFP LAPSSKSTSGGTAALGCL
VKDYFPEPVTVSWNSGALTSGVHTFPAVLQSSGLYSLSSVVTVPSSS LG
TQTYICNVNHKPSNTKVDKKVEPKSCDKTHTCPPCPAPEAAGGPSVFLFP
PKPKDTLMISRTP EVTCVVVDVSHEDPEVKFNWYVDGVEVHNAKTKPRE
EQYNSTYRVVSVLTVLHQDWLNGKEYKCK VSNKALPAPIEKTISKAKGQ
PREPQVYTLPPSRDELTKNQVSLWCLVKGFYPSDIAVEWESNGQP ENNY
KTTPPVLDSDGSFFLYSKLTVDKSRWQQGNVFSCSVMHEALHNHYTQKSL
SLSPGK

### > Seq 273: 9.49-Pembrolizumab (MHC II (Pan-Allele)-PD-1)IgG1-KIH-LALA Monovalent Bispecific 2

> Light Chain 1:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 274: 9.49-Pembrolizumab (MHC II (Pan-Allele)-PD-1)IgG1-KIH-LALA Monovalent Bispecific 2

> Light Chain 2:

EIVLTQSPATLSLSPGERATLSCRASKGVSTSGYSYLHWYQQKPGQAPRL
LIYLASYLESGVPAR FSGSGSGTDFTLTISSLEPEDFAVYYCQHSRDLP
LTFGGGTKVEIKRTVAAPSVFIFPPSDEQLK SGTASVVCLLNNFYPREA
KVQWKVDNALQSGNSQESVTEQDSKDSTYSLSSTLTLSKADYEKHKV YA
CEVTHQGLSSPVTKSFNRGEC

### > Seq 275: 9.49-Nivolumab (MHC II (Pan-Allele)-PD-1)IgG4-SPLE C-Terminal scFv Bivalent Bispecific sAg-PD-1 scFv Orientation 1

> Heavy Chain:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APCSRSTSESTAALGCLV
KDYFPEPVTVSWNSGALTSGVHTFPAVLQSSGLYSLSSVVTVPSSSL GT
KTYTCNVDHKPSNTKVDKRVESKYGPPCPSCPAPEFLGGPSVFLFPPKPK
DTLMISRTPEVTC VVVDVSQEDPEVQFNWYVDGVEVHNAKTKPREEQFN
STYRVVSVLTVLHQDWLNGKEYKCKVSNK GLPSSIEKTISKAKGQPREP
QVYTLPPSQEEMTKNQVSLTCLVKGFYPSDIAVEWESNGQPENNY KTTP
PVLDSDGSFFLYSRLTVDKSRWQEGNVFSCSVMHEALHNHYTQKSLSLSL
GKGGGGAGGGG QVQLVESGGGVVQPGRSLRLDCKASGITFSNSGMHWVR
QAPGKGLEWVAVIWYDGSKRYYADSVK GRFTISRDNSKNTLFLQMNSLR
AEDTAVYYCATNDDYWGQGTLVTVSSGGGGSGGGGSGGGGSEI VLTQSP
ATLSLSPGERATLSCRASQSVSSYLAWYQQKPGQAPRLLIYDASNRATGI
PARFSGSGS GTDFTLTISSLEPEDFAVYYCQQSSNWPRTFGQGTKVEIK

### > Seq 276: 9.49-Nivolumab (MHC II (Pan-Allele)-PD-1)IgG4-SPLE C-Terminal scFv Bivalent Bispecific sAg-PD-1 scFv Orientation 1

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 277: 9.49-Nivolumab IgG4-SPLE C-Terminal scFv Bivalent Bispecific sAg-PD-1 scFv Orientation 2

> Heavy Chain:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APCSRSTSESTAALGCLV
KDYFPEPVTVSWNSGALTSGVHTFPAVLQSSGLYSLSSVVTVPSSSL GT
KTYTCNVDHKPSNTKVDKRVESKYGPPCPSCPAPEFLGGPSVFLFPPKPK
DTLMISRTPEVTC VVVDVSQEDPEVQFNWYVDGVEVHNAKTKPREEQFN
STYRVVSVLTVLHQDWLNGKEYKCKVSNK GLPSSIEKTISKAKGQPREP
QVYTLPPSQEEMTKNQVSLTCLVKGFYPSDIAVEWESNGQPENNY KTTP
PVLDSDGSFFLYSRLTVDKSRWQEGNVFSCSVMHEALHNHYTQKSLSLSL
GKGGGGAGGGG EIVLTQSPATLSLSPGERATLSCRASQSVSSYLAWYQQ
KPGQAPRLLIYDASNRATGIPARFSGS GSGTDFTLTISSLEPEDFAVYY
CQQSSNWPRTFGQGTKVEIKGGGGSGGGGSGGGGSQVQLVESG GGVVQP
GRSLRLDCKASGITFSNSGMHWVRQAPGKGLEWVAVIWYDGSKRYYADSV
KGRFTISRD NSKNTLFLQMNSLRAEDTAVYYCATNDDYWGQGTLVTVSS

### > Seq 278: 9.49-Nivolumab (MHC II (Pan-Allele)-PD-1)IgG4-SPLE C-Terminal scFv Bivalent Bispecific sAg-PD-1 scFv Orientation 2

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 279: 9.49-Nivolumab IgG4-SPLE C-Terminal scFv Bivalent Bispecific PD-1-sAg scFv Orientation 1

> Heavy Chain:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPCSRS TSESTAALGCLVKDYFPE
PVTVSWNSGALTSGVHTFPAVLQSSGLYSLSSVVTVPSSSLGTKTYT CN
VDHKPSNTKVDKRVESKYGPPCPSCPAPEFLGGPSVFLFPPKPKDTLMIS
RTPEVTCVVVDVS QEDPEVQFNWYVDGVEVHNAKTKPREEQFNSTYRVV
SVLTVLHQDWLNGKEYKCKVSNKGLPSSI EKTISKAKGQPREPQVYTLP
PSQEEMTKNQVSLTCLVKGFYPSDIAVEWESNGQPENNYKTTPPV LDSD
GSFFLYSRLTVDKSRWQEGNVFSCSVMHEALHNHYTQKSLSLSLGKGGGG
AGGGGEVQLQE SGPSLVRPSQTLSLTCSVTGDSITSSYWNWIRKFPGNK
LEYMGYITYSGNTYYTPSLKSRISITR DTSKNQYFLQLNSVTTEDTATY
YCSRDYGRGDYAMVYWGQGTSVTVSSGGGGSGGGGSGGGGSDV VLTQSP
ATLSVTPGDSVSLSCRASQSIINNLHWYHQKSHESPRLLIKYASQSISGI
PSRFSGSGS GTDFTLSINSVETEDFGVYFCQQSGSWPYTFGGGSNLEIK

### > Seq 280: 9.49-Nivolumab (MHC II (Pan-Allele)-PD-1)IgG4-SPLE C-Terminal scFv Bivalent Bispecific PD-1-sAg scFv Orientation 1

> Light Chain:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 281: 9.49-Nivolumab IgG4-SPLE C-Terminal scFv Bivalent Bispecific PD1-sAg scFv Orientation 2

> Heavy Chain:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPCSRS TSESTAALGCLVKDYFPE
PVTVSWNSGALTSGVHTFPAVLQSSGLYSLSSVVTVPSSSLGTKTYT CN
VDHKPSNTKVDKRVESKYGPPCPSCPAPEFLGGPSVFLFPPKPKDTLMIS
RTPEVTCVVVDVS QEDPEVQFNWYVDGVEVHNAKTKPREEQFNSTYRVV
SVLTVLHQDWLNGKEYKCKVSNKGLPSSI EKTISKAKGQPREPQVYTLP
PSQEEMTKNQVSLTCLVKGFYPSDIAVEWESNGQPENNYKTTPPV LDSD
GSFFLYSRLTVDKSRWQEGNVFSCSVMHEALHNHYTQKSLSLSLGKGGGG
AGGGGDVVLTQ SPATLSVTPGDSVSLSCRASQSIINNLHWYHQKSHESP
RLLIKYASQSISGIPSRFSGSGSGTDF TLSINSVETEDFGVYFCQQSGS
WPYTFGGGSNLEIKGGGGSGGGGSGGGGSEVQLQESGPSLVRP SQTLSL
TCSVTGDSITSSYWNWIRKFPGNKLEYMGYITYSGNTYYTPSLKSRISIT
RDTSKNQYF LQLNSVTTEDTATYYCSRDYGRGDYAMVYWGQGTSVTVSS

### > Seq 282: 9.49-Nivolumab (MHC II (Pan-Allele)-PD-1)IgG4-SPLE C-Terminal scFv Bivalent Bispecific PD1-sAg scFv Orientation 2

> Light Chain:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 283: 9.49-Nivolumab IgG1-KIH-LALA C-Terminal scFv PD1 Monovalent, sAg Bivalent Bispecific 1

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGKGGGGAG GGGQVQLVESGGGVVQPGRSLRLDCKASGITFSNSGMH
WVRQAPGKGLEWVAVIWYDGSKRYYAD SVKGRFTISRDNSKNTLFLQMN
SLRAEDTAVYYCATNDDYWGQGTLVTVSSGGGGSGGGGSGGGG SEIVLT
QSPATLSLSPGERATLSCRASQSVSSYLAWYQQKPGQAPRLLIYDASNRA
TGIPARFSG SGSGTDFTLTISSLEPEDFAVYYCQQSSNWPRTFGQGTKV
EIK

### > Seq 284: 9.49-Nivolumab IgG1-KIH-LALA C-Terminal scFv PD1 Monovalent, sAg Bivalent Bispecific 1

> Heavy Chain 2:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 285: 9.49-Nivolumab (MHC II (Pan-Allele)-PD-1)IgG1-KIH-LALA C-Terminal scFv PD1 Monovalent, sAg Bivalent Bispecific 1

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 286: 9.49-Nivolumab IgG1-KIH-LALA C-Terminal scFv PD1 Monovalent, sAg Bivalent Bispecific 2

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGKGGGGAG GGGEIVLTQSPATLSLSPGERATLSCRASQSVSSYLAW
YQQKPGQAPRLLIYDASNRATGIPARF SGSGSGTDFTLTISSLEPEDFA
VYYCQQSSNWPRTFGQGTKVEIKGGGGSGGGGSGGGGSQVQLV ESGGGV
VQPGRSLRLDCKASGITFSNSGMHWVRQAPGKGLEWVAVIWYDGSKRYYA
DSVKGRFTI SRDNSKNTLFLQMNSLRAEDTAVYYCATNDDYWGQGTLVT
VSS

### > Seq 286: 9.49-Nivolumab IgG1-KIH-LALA C-Terminal scFv PD1 Monovalent, sAg Bivalent Bispecific 2

> Heavy Chain 2:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 287: 9.49-Nivolumab (MHC II (Pan-Allele)-PD-1)IgG1-KIH-LALA C-Terminal scFv PD1 Monovalent, sAg Bivalent bispecific 2

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 288: 9.49-Nivolumab IgG1-KIH-LALA C-Terminal scFv PD1 Monovalent, sAg Bivalent Bispecific 3

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGKGGGGAG GGGQVQLVESGGGVVQPGRSLRLDCKASGITFSNSGMH
WVRQAPGKGLEWVAVIWYDGSKRYYAD SVKGRFTISRDNSKNTLFLQMN
SLRAEDTAVYYCATNDDYWGQGTLVTVSSGGGGSGGGGSGGGG SEIVLT
QSPATLSLSPGERATLSCRASQSVSSYLAWYQQKPGQAPRLLIYDASNRA
TGIPARFSG SGSGTDFTLTISSLEPEDFAVYYCQQSSNWPRTFGQGTKV
EIK

### > Seq 289: 9.49-Nivolumab IgG1-KIH-LALA C-Terminal scFv PD1 Monovalent, sAg Bivalent Bispecific 3

> Heavy Chain 2:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 290: 9.49-Nivolumab (MHC II (Pan-Allele)-PD-1)IgG1-KIH-LALA C-Terminal scFv PD1 Monovalent, sAg Bivalent Bispecific 3

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 291: 9.49-Nivolumab IgG1-KIH-LALA C-Terminal scFv PD1 Monovalent, sAg Bivalent Bispecific 4

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGKGGGGAG GGGEIVLTQSPATLSLSPGERATLSCRASQSVSSYLAW
YQQKPGQAPRLLIYDASNRATGIPARF SGSGSGTDFTLTISSLEPEDFA
VYYCQQSSNWPRTFGQGTKVEIKGGGGSGGGGSGGGGSQVQLV ESGGGV
VQPGRSLRLDCKASGITFSNSGMHWVRQAPGKGLEWVAVIWYDGSKRYYA
DSVKGRFTI SRDNSKNTLFLQMNSLRAEDTAVYYCATNDDYWGQGTLVT
VSS

### > Seq 292: 9.49-Nivolumab (MHC II (Pan-Allele)-PD-1)IgG1-KIH-LALA C-Terminal scFv PD1 Monovalent, sAg Bivalent Bispecific 4

> Heavy Chain 2:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 293: 9.49-Nivolumab (MHC II (Pan-Allele)-PD-1)IgG1-KIH-LALA C-Terminal scFv PD1 Monovalent, sAg Bivalent bispecific 4

> Light Chain:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 294: 9.49-Nivolumab IgG1-KIH-LALA Monovalent Bispecific 1

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 295: 9.49-Nivolumab (MHC II (Pan-Allele)-PD-1)IgG1-KIH-LALA Monovalent Bispecific 1

> Heavy Chain 2:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPSSKS TSGGTAALGCLVKDYFPE
PVTVSWNSGALTSGVHTFPAVLQSSGLYSLSSVVTVPSSSLGTQTYI CN
VNHKPSNTKVDKKVEPKSCDKTHTCPPCPAPEAAGGPSVFLFPPKPKDTL
MISRTPEVTCVVV DVSHEDPEVKFNWYVDGVEVHNAKTKPREEQYNSTY
RVVSVLTVLHQDWLNGKEYKCKVSNKALP APIEKTISKAKGQPREPQVY
TLPPSRDELTKNQVSLWCLVKGFYPSDIAVEWESNGQPENNYKTT PPVL
DSDGSFFLYSKLTVDKSRWQQGNVFSCSVMHEALHNHYTQKSLSLSPGK

### > Seq 296: 9.49-Nivolumab (MHC II (Pan-Allele)-PD-1)IgG1-KIH-LALA Monovalent Bispecific 1

> Light Chain 1:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 297: 9.49-Nivolumab (MHC II (Pan-Allele)-PD-1)IgG1-KIH-LALA Monovalent Bispecific 1

> Light Chain 2:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 298: 9.49-Nivolumab IgG1-KIH-LALA Monovalent Bispecific 2

> Heavy Chain 1:

EVQLQESGPSLVRPSQTLSLTCSVTGDSITSSYWNWIRKFPGNKLEYMGY
ITYSGNTYYTPSLKS RISITRDTSKNQYFLQLNSVTTEDTATYYCSRDY
GRGDYAMVYWGQGTSVTVSSASTKGPSVFPL APSSKSTSGGTAALGCLV
KDYFPEPVTVSWNSGALTSGVHTFPAVLQSSGLYSLSSVVTVPSSSL GT
QTYICNVNHKPSNTKVDKKVEPKSCDKTHTCPPCPAPEAAGGPSVFLFPP
KPKDTLMISRTPE VTCVVVDVSHEDPEVKFNWYVDGVEVHNAKTKPREE
QYNSTYRVVSVLTVLHQDWLNGKEYKCKV SNKALPAPIEKTISKAKGQP
REPQVYTLPPSRDELTKNQVSLWCLVKGFYPSDIAVEWESNGQPE NNYK
TTPPVLDSDGSFFLYSKLTVDKSRWQQGNVFSCSVMHEALHNHYTQKSLS
LSPGK

### > Seq 299: 9.49-Nivolumab (MHC II (Pan-Allele)-PD-1)IgG1-KIH-LALA Monovalent Bispecific 2

> Heavy Chain 2:

QVQLVESGGGVVQPGRSLRLDCKASGITFSNSGMHWVRQAPGKGLEWVAV
IWYDGSKRYYADSVK GRFTISRDNSKNTLFLQMNSLRAEDTAVYYCATN
DDYWGQGTLVTVSSASTKGPSVFPLAPSSKS TSGGTAALGCLVKDYFPE
PVTVSWNSGALTSGVHTFPAVLQSSGLYSLSSVVTVPSSSLGTQTYI CN
VNHKPSNTKVDKKVEPKSCDKTHTCPPCPAPEAAGGPSVFLFPPKPKDTL
MISRTPEVTCVVV DVSHEDPEVKFNWYVDGVEVHNAKTKPREEQYNSTY
RVVSVLTVLHQDWLNGKEYKCKVSNKALP APIEKTISKAKGQPREPQVY
TLPPSRDELTKNQVSLWCLVKGFYPSDIAVEWESNGQPENNYKTT PPVL
DSDGSFFLYSKLTVDKSRWQQGNVFSCSVMHEALHNHYTQKSLSLSPGK

### > Seq 300: 9.49-Nivolumab (MHC II (Pan-Allele)-PD-1)IgG1-KIH-LALA Monovalent Bispecific 2

> Light Chain 1:

DVVLTQSPATLSVTPGDSVSLSCRASQSIINNLHWYHQKSHESPRLLIKY
ASQSISGIPSRFSGS GSGTDFTLSINSVETEDFGVYFCQQSGSWPYTFG
GGSNLEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

### > Seq 301: 9.49-Nivolumab (MHC II (Pan-Allele)-PD-1)IgG1-KIH-LALA Monovalent Bispecific 2

> Light Chain 2:

EIVLTQSPATLSLSPGERATLSCRASQSVSSYLAWYQQKPGQAPRLLIYD
ASNRATGIPARFSGS GSGTDFTLTISSLEPEDFAVYYCQQSSNWPRTFG
QGTKVEIKRTVAAPSVFIFPPSDEQLKSGTA SVVCLLNNFYPREAKVQW
KVDNALQSGNSQESVTEQDSKDSTYSLSSTLTLSKADYEKHKVYACE VT
HQGLSSPVTKSFNRGEC

It is to be understood that each of these LoSTIM constructs can be further engineered using standard and well-known methods in the art and described herein to further comprise sequences of interest described herein, including, without limitation, humanization, antibody formats of interest, engineered Fc domains, linkers, and the like.

Numerous designs and strategies for producing bispecific antibodies described herein are contemplated and may be applied to any embodiment in any combination. For example, some embodiments include Fc domains and others do not, this results in advantages and disadvantages from a pharmacokinetic and drug distribution standpoint. Some embodiments are multivalent, that is they have multiple binding sites for each antigen, and some are monovalent, that is they have a single binding site for each antigen.

Some embodiments have a single binding site for one antigen and multiple binding sites for the other antigen. Some embodiments involve asymmetric pairing of heavy chains using “knob-in-hole” or electrostatic steering techniques and others involve linking single-chain variable fragments (scFv) to aminotermini or carboxytermini of the heavy or light chains. A diversity of artificial linkers can be used to connect scFv to the heavy or light chains, such glycine-serine and glycine-alanine linkers. Variable regions for the two binding partners can also be connected to one another without an Fc domain in a variety of orientations by artificial linkers, resulting in designs such as diabodies and “bi-specific T-cell engagers.” The plethora of bispecific designs is the subject of a number of review articles, which include a more comprehensive listing of known bispecific antibody designs (see, for example, Brinkmann and Kontermann (2017) MABS 9(2): 182-212).

In some embodiments, the cell type-specific marker is MHC class II. MHC class II is a cell type-specific marker for professional antigen presenting cells (APCs). Other cells or tissue types do not express this marker. Thus, a class II MHC-specific LoSTIM selectively prevents T-cell activation by these cells, which prevents or inhibits T-cell activation by professional APCs and resulting in inhibition of T-cell priming and inhibition of T-cell responses against antigens that are being presented by those APCs. Another result is inhibition of B-cell class-switching and affinity maturation, since B-cells are professional APCs that express MHC class II and T-cell help is necessary for these processes, which requires direct interaction with T-cells. Thus, an MHC class II-specific LoSTIM may be used as an immunosuppressant to engender T-cell (cell mediated) and B-cell (humoral) tolerance against antigens. These may be antigens that are naturally presented by the body during autoimmune disease or undesirable allogeneic immune responses, such as in organ transplantation. Similarly, antigens may be exogenously administered concurrently with an MHC class II-specific LoSTIM to engender immunologic tolerance against those antigens.

### Example 4: LoSTIM Agents May Be Used to Treat Clinical Indications

LoSTIM agents may be used to treat subjects at risk of, suspected of having, or afflicted with a variety of clinical indications depending on the LoSTIM’s design, as described herein, such as cancer, autoimmune disease, GVHD, irAE, and the like, using routine procedures described herein and well-known in the art.

For example, colitis is a common irAE following PD-1/PD-L1 inhibition (Alessandrino et al. (2019) Abdom. Radiol. (NY) 44(5):1917-1927). A variant of the experimental autoimmune colitis model is dependent upon CD8 T-cell activity (Punit et al. (2015) Gastroenterol. 149(4):993-1005; Tajima et al. (2008) J. Exp. Med. 205:1019-1027). CD8⁺C44^{low}CD62L⁺ cells are purified from C57BL/6 peripheral nodes via flow cytometry sorting and adoptively transferred to immunodeficient C57BL/6 Rag2-/- recipients as previously described in Punit et al. (2015) Gastroenterol. 149(4):993-1005. The onset of colitis typically occurs 4-5 weeks after adoptive transfer. Either before the onset (prophylactic studies) or after the onset (therapeutic studies), treatment is initiatied with an H-2Kb-specific LoSTIM, AF6-88.5.3 (parental anti-H-2Kb), 29F.1A12 (parental anti-PD1), or PBS. This experiment is performed separately with an EpCAM-specific LoSTIM along with appropriate controls including parent anti-EpCAM monospecific and parent anti-PD1 monospecific antibodies and PBS as a vehicle control. Since C57BL/6 tissues express H-2K^{b} and also express EpCAM, the aforementioned LoSTIMs will bind to their surface and are expected to inhibit T-cell activity and ameliorate immune-mediated colitis in this model.

### Example 5: Materials and Methods

### Design and Production of the LoSTIM

The proof-of-concept LoSTIM was created in a bispecific antibody format. To do so, first the sequence of the variable regions of the heavy chain (V_{H}) and light chain (V_{L}) of the AF6-88.5.3 hybridoma were determined, which expresses a mouse IgG2a kappa anti-H-2K^{b} monoclonal antibody. The AF6-88.5.3 hybridoma was obtained from the American Type Culture Collection (ATCC). cDNAs containing the V_{H} and V_{L} were prepared by Rapid Amplification of cDNA Ends (RACE). The resultant antibody variable region cDNA fragments were cloned into a TOPO vector (LakePharma, Inc). A total of 14 clones were amplified by polymerase chain reaction (PCR) and purified by gel electrophoresis and recovery. Each amplified and purified clone was subjected to Sanger sequencing, resulting in a single V_{H} and a single V_{L} consensus sequence.

The LoSTIM bispecific antibody protein sequence was designed as follows. The amino acid translation of the AF6-88.5.3 V_{H} sequence was appended to the aminoterminus of an amino acid sequence of the mouse IgG2a CH1 domain, hinge region, and Fc domain. The Fc domain included point substitutions known to decrease Fcγ receptor binding and C1q binding: L235E, E318A, K320A, and K322A (Duncan et al. (1988) Nature 332(6164):563-4; Duncan and Winter (1988) Nature 332(6166):738-40; Steurer et al. (1995) J Immunol. 155(3):1165-74). To create a LoSTIM capable of binding to both H-2K^{b} and PD-1, a single-chain variable fragment (scFv) sequence containing the amino acid sequences of the variable regions of the anti-PD-1 monoclonal antibody 29F.1A12 was constructed and appended to the carboxyterminus of the Fc domain, with the two sequences separated by the flexible linker sequence GGGGAGGGG (SEQ ID NO: 16). The V_{H} and V_{L} sequences of 29F.1A12 had been previously obtained by the method described in the preceding paragraph. To create the scFv sequence, the 29F.1A12 V_{L} sequence was appended to the aminoterminus of the 29F.1A12 V_{H} sequence, with the two sequences being separated by the flexible linker sequence GGGGSGGGGSGGGGS (SEQ ID NO: 17). To create the light chain amino acid sequence, the AF6-88.5.3 V_{L} was appended directly to the aminoterminus of the mouse kappa-chain constant region. The nucleotide sequences encoding the heavy chain and light chain of the LoSTIM were obtained by computational reverse-transcription and codon optimization. A nucleotide sequence encoding the signal peptide MEWSWVFLFFLSVTTGVHS (SEQ ID NO: 18) was appended immediately 5′ to both the heavy and light chain nucleotide sequences.

AF6-88.5.3 V_{H} nucleotide sequence (SEQ ID NO: 19)

GATGTGCAGCTGGTGGAGTCTGGGGGAGGCTTAGTGCAGCCTGGAGGGTC
CCGGAAACTCTCCTGTGCAGCCTCTGGATTCACTTTCAGTAGCTTTGGAA
TGCACTGGGTTCGTCAGGCTCCAGAGAAGGGGCTGGAGTGGGTCGCATAC
ATTAGTAGTGGCAGTAATACCATCTACTATGCAGACACAGTGAAGGGCCG
ATTCACCATCTCCAGAGACAATCCCAAGAACACCCTGTTCCTGCAAATGA
CCAGTCTAAGGTCTGAGGACACGGCCATGTATTATTGTGCACTTACGACG
GGGTCCTGGTTTGCTTACTGGGGCCAAGGGACTCTGGTCACTGTCTCTGC
A

AF6-88.5.3 V_{L} nucleotide sequence (SEQ ID NO: 20)

GACATCTTGCTGACTCAGTCTCCAGCCATCCTGTCTGTGCGTCCAGGAGA
AAGAGTCAGTTTCTCCTGCAGGGCCAGTCAGAGCCTTGGCACAAGCATAC
ACTGGTATCAGCAAAGAACAGATGGTTCTCCAAGGCTTCTCATAAAATAT
GCTTCTGAGTCTATCTCTGGGATCCCTTCCAGGTTTAGTGGCAGTGGATC
AGGGACAGATTTTACTCTTAGCATCAACAGTGTGGAGTCTGAAGATATTG
CAGATTATTACTGTCAACAAAGTAATAGCTGGCCAATCACGTTCGGTGCT
GGGACCAAGCTGGAGCTGAAA

AF6-88.5.3 V_{H} amino acid sequence (SEQ ID NO: 21)

DVQLVESGGGLVQPGGSRKLSCAASGFTFSSFGMHWVRQAPEKGLEWVAY
ISSGSNTIYYADTVKGRFTISRDNPKNTLFLQMTSLRSEDTAMYYCALTT
GSWFAYWGQGTLVTVSA

AF6-88.5.3 V_{L} amino acid sequence (SEQ ID NO: 22)

DILLTQSPAILSVRPGERVSFSCRASQSLGTSIHWYQQRTDGSPRLLIKY
ASESISGIPSRFSGSGSGTDFTLSINSVESEDIADYYCQQSNSWPITFGA
GTKLELK

mIgG2a CH1, hinge region, and Fc domain including L235E, E318A, K320A, and K322A substitutions (SEQ ID NO: 23)

AKTTAPSVYPLAPVCGDTTGSSVTLGCLVKGYFPEPVTLTWNSGSLSSGV
HTFPAVLQSDLYTLSSSVTVTSSTWPSQSITCNVAHPASSTKVDKKIEPR
GPTIKPCPPCKCPAPNLE GGPSVFIFPPKIKDVLMISLSPIVTCVVVDVS
EDDPDVQISWFVNNVEVHTAQTQTHREDYNSTLRVVSALPIQHQDWMSGK A FA CA VNNKDLPAPIERTISKPKGSVRAPQVYVLPPPEEEMTKKQVTLTC
MVTDFMPEDIYVEWTNNGKTELNYKNTEPVLDSDGSYFMYSKLRVEKKNW
VERNSYSCSVVHEGLHNHHTTKSFSRTPGK

29F.1A12 scFv sequence (SEQ ID NO: 24)

DVALTQTPVAQPVTLGDQASISCRSSQSLVHSNGRTYLEWYLQKPGQSPQ
LLIYKVSNRFSGVPDRFIGSGSGSDFTLTISRVEPEDLGVYYCFQATHDP
NTFGAGTKLELKGGGGSGGGGSGGGGSQVQLQQSGAELVKPGSSVKISCK
ASGYTFTSHFIHWIKQQPGNGLEWIGGIYPGDGDTEYNQQFNGKATLTAD
KSSSTAYMRLSSLTSEDSAVYFCATRVPSYWFFDFWGPGTMVTVSS

Full heavy chain amino acid sequence of the LoSTIM heavy chain, including leader sequence (SEQ ID NO: 25)

MEWSWVFLFFLSVTTGVHSDVQLVESGGGLVQPGGSRKLSCAASGFTFSS
FGMHWVRQAPEKGLEWVAYISSGSNTIYYADTVKGRFTISRDNPKNTLFL
QMTSLRSEDTAMYYCALTTGSWFAYWGQGTLVTVSAAKTTAPSVYPLAPV
CGDTTGSSVTLGCLVKGYFPEPVTLTWNSGSLSSGVHTFPAVLQSDLYTL
SSSVTVTSSTWPSQSITCNVAHPASSTKVDKKIEPRGPTIKPCPPCKCPA
PNLEGGPSVFIFPPKIKDVLMISLSPIVTCVVVDVSEDDPDVQISWFVNN
VEVHTAQTQTHREDYNSTLRVVSALPIQHQDWMSGKAFACAVNNKDLPAP
IERTISKPKGSVRAPQVYVLPPPEEEMTKKQVTLTCMVTDFMPEDIYVEW
TNNGKTELNYKNTEPVLDSDGSYFMYSKLRVEKKNWVERNSYSCSVVHEG
LHNHHTTKSFSRTPGKGGGGAGGGGDVALTQTPVAQPVTLGDQASISCRS
SQSLVHSNGRTYLEWYLQKPGQSPQLLIYKVSNRFSGVPDRFIGSGSGSD
FTLTISRVEPEDLGVYYCFQATHDPNTFGAGTKLELKGGGGSGGGGSGGG
GSQVQLQQSGAELVKPGSSVKISCKASGYTFTSHFIHWIKQQPGNGLEWI
GGIYPGDGDTEYNQQFNGKATLTADKSSSTAYMRLSSLTSEDSAVYFCAT
RVPSYWFFDFWGPGTMVTVSS

Full light chain amino acid sequence of the LoSTIM, including leader sequence (SEQ ID NO: 26)

MEWSWVFLFFLSVTTGVHSDILLTQSPAILSVRPGERVSFSCRASQSLGT
SIHWYQQRTDGSPRLLIKYASESISGIPSRFSGSGSGTDFTLSINSVESE
DIADYYCQQSNSWPITFGAGTKLELKRADAAPTVSIFPPSSEQLTSGGAS
VVCFLNNFYPKDINVKWKIDGSERQNGVLNSWTDQDSKDSTYSMSSTLTL
TKDEYERHNSYTCEATHKTSTSPIVKSFNRNEC

Full nucleotide sequence of the LoSTIM heavy chain, including leader sequence (SEQ ID NO: 27)

ATGGAATGGAGCTGGGTCTTTCTCTTCTTCCTGTCAGTAACGACTGGTGT
CCACTCCGACGTGCAGCTGGTGGAGAGCGGCGGTGGCCTGGTGCAGCCCG
GAGGTAGCAGGAAGCTGAGCTGCGCCGCAAGCGGCTTCACCTTTAGCAGC
TTCGGCATGCACTGGGTGAGGCAAGCCCCCGAGAAGGGCCTGGAGTGGGT
GGCATACATCAGCAGCGGCAGCAACACCATCTACTACGCCGACACCGTGA
AGGGCAGGTTCACCATTAGCAGGGACAACCCCAAAAACACCCTGTTCCTG
CAGATGACCAGCCTGAGGAGCGAGGACACCGCCATGTACTACTGCGCCCT
GACCACCGGCTCTTGGTTTGCCTACTGGGGCCAGGGCACCCTGGTGACCG
TGAGCGCCGCTAAAACAACAGCCCCATCGGTCTATCCACTGGCCCCTGTG
TGTGGAGATACAACTGGCTCCTCGGTGACTCTAGGATGCCTGGTCAAGGG
TTATTTCCCTGAGCCAGTGACCTTGACCTGGAACTCTGGTTCCCTGTCCA
GTGGTGTGCACACCTTCCCAGCTGTCCTGCAGTCTGACCTCTACACCCTC
AGCTCAAGCGTGACTGTAACCAGCTCGACCTGGCCCAGCCAGTCCATCAC
CTGCAATGTGGCCCACCCGGCAAGCAGCACCAAGGTGGACAAGAAAATTG
AGCCCAGAGGGCCCACAATCAAGCCCTGTCCTCCATGCAAATGCCCAGCA
CCTAACCTCGAGGGTGGACCATCCGTCTTCATCTTCCCTCCAAAGATCAA
GGATGTACTCATGATCTCCCTGAGCCCCATAGTCACATGTGTAGTCGTTG
ATGTGAGCGAGGATGACCCAGATGTCCAGATCAGCTGGTTTGTGAACAAC
GTGGAAGTGCACACTGCTCAGACACAGACGCATAGAGAGGATTACAACAG
TACTCTCCGGGTTGTCAGTGCCCTCCCCATCCAGCACCAGGACTGGATGA
GTGGCAAGGCCTTCGCATGCGCTGTCAACAACAAAGACCTCCCAGCGCCC
ATCGAGAGAACCATCTCAAAACCCAAAGGGTCAGTAAGAGCTCCACAGGT
ATATGTCTTGCCTCCACCAGAAGAGGAGATGACTAAGAAACAGGTCACTC
TGACCTGCATGGTCACAGACTTCATGCCTGAAGACATTTACGTGGAGTGG
ACCAACAACGGGAAAACAGAGCTAAACTACAAGAACACTGAACCAGTCCT
GGACTCTGATGGTTCTTACTTCATGTACAGCAAGCTGAGAGTGGAGAAGA
AGAACTGGGTGGAGAGAAATAGCTACTCCTGTTCAGTGGTCCACGAGGGT
CTGCACAATCACCACACGACTAAGAGCTTCTCCCGGACTCCGGGTAAGGG
CGGAGGTGGTGCAGGAGGCGGTGGAGACGTTGCCCTGACACAGACCCCCG
TGGCCCAGCCCGTGACCCTGGGCGACCAGGCCAGCATCAGCTGCAGGAGC
AGCCAGAGCTTGGTTCACAGCAACGGCAGGACCTACCTGGAGTGGTACCT
GCAGAAGCCGGGACAGAGCCCCCAGCTGCTGATCTACAAGGTGAGCAATA
GGTTCAGCGGCGTGCCCGACAGGTTCATAGGCAGTGGAAGCGGCAGCGAT
TTCACCCTGACCATTAGCAGGGTGGAGCCCGAGGACCTGGGAGTGTACTA
CTGCTTCCAGGCAACCCACGACCCCAACACCTTCGGCGCTGGAACTAAGC
TGGAGCTGAAGGGTGGCGGAGGATCTGGCGGAGGCGGTAGTGGCGGTGGC
GGATCTCAGGTGCAGCTCCAGCAGAGCGGTGCCGAGCTGGTTAAGCCCGG
TAGCAGCGTGAAGATCAGCTGCAAGGCCAGCGGCTACACCTTCACCAGCC
ACTTCATCCACTGGATCAAACAGCAGCCCGGCAACGGCCTGGAGTGGATC
GGCGGCATCTACCCCGGAGACGGCGACACCGAGTACAATCAGCAGTTCAA
CGGAAAGGCCACCCTGACCGCCGACAAGAGCAGCAGCACCGCCTACATGA
GGCTGAGCAGCCTGACCAGCGAGGACAGCGCCGTGTACTTCTGCGCCACC
AGGGTGCCCAGCTACTGGTTCTTCGACTTCTGGGGCCCTGGCACGATGGT
CACCGTGTCTTCTTAA

Full light chain nucleotide sequence of the LoSTIM, including leader sequence (SEQ ID NO: 28)

ATGGAATGGAGCTGGGTCTTTCTCTTCTTCCTGTCAGTAACGACTGGTGT
CCACTCCGACATCCTGCTGACCCAAAGCCCGGCAATCCTGAGCGTGAGGC
CCGGTGAGAGGGTGAGCTTCAGCTGCAGGGCCAGCCAGTCCCTGGGCACC
AGCATCCACTGGTATCAACAAAGGACCGACGGCAGCCCCAGGCTGCTGAT
CAAGTACGCCAGCGAGAGCATTAGCGGCATCCCCAGCAGGTTCAGCGGGA
GCGGCAGCGGCACCGACTTCACTCTGAGCATCAACAGCGTGGAGAGCGAG
GACATCGCCGACTACTACTGCCAGCAGAGCAACAGCTGGCCAATCACCTT
CGGCGCAGGCACCAAGCTGGAGCTGAAACGGGCAGATGCTGCACCAACTG
TATCCATCTTCCCACCATCCAGTGAGCAGTTAACATCTGGAGGTGCCTCA
GTCGTGTGCTTCTTGAACAACTTCTACCCCAAAGACATCAATGTCAAGTG
GAAGATTGATGGCAGTGAACGACAAAATGGCGTCCTGAACAGTTGGACTG
ATCAGGACAGCAAAGACAGCACCTACAGCATGAGCAGCACCCTCACGTTG
ACCAAGGACGAGTATGAACGACATAACAGCTATACCTGTGAGGCCACTCA
CAAGACATCAACTTCACCCATTGTCAAGAGCTTCAACAGGAATGAGTGTT
GATAA

The heavy chain and light chain nucleotide sequences were synthesized and cloned into the pLEV123 expression vector (LakePharma, Inc) and the sequence was confirmed by PCR amplification followed by Sanger sequencing. The vector was then amplified by PCR and the amplified DNA was run on an agarose gel for quality assessment and its sequence confirmed again by Sanger sequencing before proceeding to transfection. Protein production and purification was performed by LakePharma’s standard operating procedure. In brief, Chinese hamster ovary (CHO) cells were seeded in suspension in a shake flask and were expanded using serum-free chemically defined medium. On the day of transfection, the expanded cells were seeded into a new flask with fresh medium. The expression vector was transiently transfected into the CHO cells. The cells were maintained as a batch-fed culture for fourteen days. The conditioned media from the transient production run was harvested and clarified by centrifugation and filtration. The supernatant was loaded over a Protein A column pre-equilibrated with binding buffer. Washing buffer was passed through the column until the OD280 value (NanoDrop, Thermo Scientific) was measured to be zero. The target protein was eluted with a low pH buffer, fractions were collected, and the OD280 value of each fraction was recorded. Fractions containing the target protein were pooled and filtered through a 0.2 µm membrane filter. The protein concentration was calculated from the OD280 value and the calculated extinction coefficient. Capillary electrophoresis (CE-SDS) analysis of the target protein was performed using LabChip GXII (Perkin Elmer). To confirm a low endotoxin level, duplicate samples of the purified product were quantified using the chromogenic Limulus Amebocyte Lysate method (Endosafe-MCS, Charles River Laboratories).

### Binding Assays

Binding of the LoSTIM to PD-1 was assessed by flow cytometry using cells that express PD-1. 300-mPD-1 cells, transgenic mouse pro-B cells that stably express mouse PD-1, were exposed to serial dilutions of LoSTIM in phosphate-buffered saline with 0.5% bovine serum albumin (PBS-BSA) for 15 minutes at room temperature. They were then washed three times with PBS-BSA and incubated with a secondary antibody, anti-mouse IgG2a conjugated to phycoerythrin (anti-mouse IgG2a-PE) (Southern Biotech), for 10 minutes at 4° C. Cells were then washed three times with PBS-BSA. The cells were then analyzed by flow cytometry using an LSR Fortessa flow cytometer (Beckton Dickenson) and mean fluorescence intensity of PE was used as a metric of LoSTIM binding.

To confirm binding specificity, binding to activated primary T cells that express PD-1 was compared with binding to naive primary T cells that do not express PD-1. These T cells were obtained from a background that does not express H-2K^{b}, BALB/cByJ. These mice were obtained from the Jackson Laboratory (Bar Harbor, Maine) were sacrificed and a necropsy was performed to remove their spleens. Spleens were washed with sterile RPMI-1640 media (Thermo Fisher Scientific) with 10% fetal calf serum, penicillin, and streptomycin, hereafter referred to as complete media and then mechanically disaggregated in complete media to release splenocytes and filtered using a 40 micron sterile filter. Red blood cells were lysed by incubation in Red Hybri-Max Blood Cell Lysing Buffer (Millipore Sigma) for 5 minutes, followed by dilution and wash in complete media. The red blood cell lysis procedure was repeated one to two times until the cell pellet was white, indicating complete red blood cell lysis. Splenocytes were then counted and CD4 T cells were purified by negative magnetic selection using the CD4+ T Cell Isolation Kit (Miltenyi Biotech) according to its protocol. On the day prior to splenocyte harvest and T cell purification, a 10 cm tissue culture treated plate (Corning) was coated with anti-CD3 clone 17A2 (BioXCell) and the plate was incubated overnight at 4 degrees Celcius and then washed twice with PBS. After T cell purification, half of the purified T cells were activated by incubation in the anti-CD3 antibody-coated plate in the presence of soluble anti-CD28 clone 37.51 (BioXCell), and the other T cells were incubated in a plate that was not coated with antibody. After three days of incubation, both activated and rested naive T cells were washed with PBS-BSA and exposed to varying concentrations of LoSTIM in PBS-BSA for 15 minutes. Cells were then washed three times with PBS-BSA and incubated with anti-mouse-IgG2a-PE for 10 minutes at 4° C. Cells were washed three times in PBS-BSA and then analyzed by flow cytometry using an LSR Fortessa flow cytometer.

Binding to H-2Kb was assessed by flow cytometry using the C57BL/6 colorectal cancer cell line MC-38, which are known to express H-2Kb, and compared with binding to CT-26 cells, which are known not to express H-2Kb. Neither MC-38 nor CT-26 cells express PD-1. Cells were washed with PBS-BSA and exposed to varying concentrations of LoSTIM in PBS-BSA for 15 minutes. Cells were then washed three times with PBS-BSA and incubated with anti-mouse-IgG2a-PE for 10 minutes at 4° C. Cells were washed three times in PBS-BSA and then analyzed by flow cytometry using an LSR Fortessa flow cytometer.

Simultaneous bispecific binding to PD-1 and H-2K^{b} was assessed by flow cytometry using MC-38 cells, which express H-2K^{b} but not PD-1. MC-38 cells were washed with PBS-BSA and exposed to varying concentrations of LoSTIM in PBS-BSA for 15 minutes. Cells were then washed three times with PBS-BSA and incubated with Recombinant Mouse PD-1 Fc Chimera Protein (R&D Systems) bearing a human IgG1 Fc domain. Cells were washed three times with PBS-BSA and then incubated with anti-human-IgG1-PE adsorbed against mouse IgG (Southern Biotech) for 10 minutes at 4° C. Cells were washed three times in PBS-BSA and then analyzed by flow cytometry using an LSR Fortessa flow cytometer. In a separate experiment, simultaneous bispecific binding to PD-1 and H-2K^{b} was examined in an experimental setup where competition between monospecific and bispecific binding was allowed. In this experiment, MC-38 cells were similarly incubated with varying concentrations of LoSTIM in PBS-BSA but Recombinant Mouse PD-1 Fc Chimera Protein was added and allowed to incubate concurrently with the LoSTIM before cells were washed with PBS-BSA. Detection was similarly performed with mouse adsorbed anti-human-IgG-PE and cells were analyzed using an LSR Fortessa flow cytometer.

### T Cell Activation Assay

Bone marrow-derived dendritic cells (BMDDC) used as antigen presenting cells (APC) were prepared as follows. C57BL/6 or C3H mice obtained from the Jackson Laboratory were sacrificed and a necropsy was performed. Femurs and tibias were isolated and cleaned in 70% ethanol. After allowing the bones to dry, a scalpel was used to remove the ends of the bone and complete media was flushed through the bones using a syringe with a fine needle to remove the bone marrow. Bone marrow was then mechanically disaggregated by repeated aspiration with a syringe and then filtered using a 40 micron sterile filter. Red blood cells were lysed by incubation in Red Hybri-Max Blood Cell Lysing Buffer for 5 minutes, followed by dilution and wash in complete media. The red blood cell lysis procedure was repeated one to two times until the cell pellet was white, indicating complete red blood cell lysis. Bone marrow cells were counted and resuspended in complete media containing 20 ng/mL GM-CSF (Biolegend) and incubated at 37° C. in 5% CO2. After three days, an equivalent volume of complete media with 20 ng/mL GM-CSF was added. Three days later, half of the culture volume was removed, centrifuged, and resuspended in fresh complete media containing 20 ng/mL GM-CSF, returned to the culture, and incubated for an additional two days. A mix of suspension and loosely adherent BMDDC cells was obtained via repeat aspiration using a pippete.

Purified B-cells were used as APCs in some experiments and were prepared as follows. C57BL/6 or C3H mice were obtained from the Jackson Laboratory, sacrificed, and a necropsy was performed to remove their spleens. Spleens were washed with complete media and then mechanically disaggregated in complete media to release splenocytes and filtered using a 40 micron sterile filter. Red blood cells were lysed by incubation in Red Hybri-Max Blood Cell Lysing Buffer for 5 minutes, followed by dilution and wash in complete media. The red blood cell lysis procedure was repeated one to two times until the cell pellet was white, indicating complete red blood cell lysis. Splenocytes were then counted and B-cells were purified by negative magnetic selection using the Pan B Cell Isolation Kit II (Miltenyi Biotech) according to its protocol.

To obtain naive T cells, BALB/cByJ mice were sacrificed and a necropsy was performed to remove their spleens. Spleens were washed with sterile RPMI-1640 media (Thermo Fisher Scientific) with complete media and then mechanically disaggregated in complete media to release splenocytes. Red blood cells were lysed by incubation in Red Hybri-Max Blood Cell Lysing Buffer for 5 minutes, followed by dilution and wash in complete media. The red blood cell lysis procedure was repeated one to two times until the cell pellet was white, indicating complete red blood cell lysis. Splenocytes were then counted and CD8 T cells were purified by negative magnetic selection using either the CD8+ T Cell Isolation Kit or CD4+ T Cell Isolation Kit (Miltenyi Biotech) according to their respective protocols.

To assess the effect of the LoSTIM on naive T cell activation by APCs, naive BALB/cByJ T cells were co-cultured with either BMDDC or B-cell APCs at a 1:1 ratio for five days. The effect of the LoSTIM on T cells primed by APCs obtained from C57BL/6 mice, which express H-2Kb and therefore bind the LoSTIM, was compared with its effect on T cells primed by APCs obtained from C3H mice, which do not express H-2Kb and therefore do not bind the LoSTIM. The detailed method was as follows. Naive BALB/cByJ T cells were obtained as described above and labeled with carboxyfluorescein succinimidyl ester (CFSE) using the CellTrace CFSE Cell Proliferation Kit (Thermo Fisher Scientific) according to its protocol. BMDDC or B-cells obtained from either C57BL/6 or C3H mice were obtained as described above and labeled with CellTrace Violet dye using CellTrace Violet Cell Proliferation Kit (Thermo Fisher Scientific) according to its protocol. 1×10⁵ T cells and 1×10⁵ APCs were then co-cultured in 200 microliters of complete media in a 96-well tissue culture plate. At least four replicate cultures were included for each condition. Treatment conditions included varying concentrations of LoSTIM (between 50 nanomolar and 500 nanomolar), parent anti-PD-1 clone 1A12 monoclonal antibody as a negative control, parent anti-H-2K^{b} clone AF6-88.5.3 monoclonal antibody as a negative control, or PBS as a vehicle control. 10 ug/mL of purified rat anti-mouse CD16/CD32 clone 2.4G2 was added to each culture to ensure that any binding of the LoSTIM or the control antibodies to the APCs reflected only H-2K^{b}-specific binding and not Fcγ receptor binding. After five days of incubation at 37° C. in an atmosphere of 5% CO2, cells were analyzed by flow cytometry using an LSR Fortessa flow cytometer. The percentage of T cells undergoing cell division was determined by calculating the percentage of CellTrace Violet negative cells with CFSE dilution and this was used as a metric of T cell activation.

### Signaling Assay

To assess the effect on T cell receptor (TCR) signaling, CD8+ T cells expressing a transgenic TCR that recognizes the ovalbumin antigen SIINFEKL (SEQ ID NO: 29) were purified from OT-1 mice, activated, and then restimulated by C57BL/6 B-cells cells loaded with SIINFEKL antigen in the presence or absence of LoSTIM. TCR activation was assessed by phosphoflow cytometry. The detailed method was as follows. OT-1 mice were obtained from the Jackson Laboratory (Bar Harbor, Maine) and sacrificed. A necropsy was performed to remove their spleens. Spleens were washed with complete media and then mechanically disaggregated in complete media to release splenocytes. Red blood cells were lysed by incubation in Red Hybri-Max Blood Cell Lysing Buffer for 5 minutes, followed by dilution and wash in complete media. The red blood cell lysis procedure was repeated one to two times until the cell pellet was white, indicating complete red blood cell lysis. Splenocytes were then counted and CD8 T cells were purified by negative magnetic selection using the CD8+ T Cell Isolation Kit (Miltenyi Biotech) according to its protocol. On the day prior to splenocyte harvest and T cell purification, a 10 cm tissue culture treated plate (Corning) was coated with anti-CD3 clone 17A2 (BioXCell) and the plate was incubated overnight at 4° C.elcius and then washed twice with PBS. Purified T cells were activated by incubation in the anti-CD3 antibody-coated plate in the presence of soluble anti-CD28 clone 37.51 at 37° C. for three days. Activated T cells were then removed from the plate, washed in complete media, and then rested in a tissue culture plate without antibody stimulation for six hours. Concurrently, fresh C57BL/6 B-cells, purified as described above, were pulsed with 10 micromolar of SIINFEKL peptide for 6 hours, washed three times, and then co-cultured in a 96 well round bottom plate (Corning) with rested T cells. Before incubation, the plate was centrifuged for one minute at 1250 rpm to bring the T cells and B cells in to close proximity. Cocultures were incubated at 37° C. and 5% CO2. Cells were fixed and permeabilized using the eBioscience Intracellular Fixation and Permeabilization Buffer Set according to the published kit protocol. Cells were stained at the appropriate points in the kit protocol with anti-phospho-ZAP70 (clone n3kobu5) conjugated to PE (Thermo Fisher Scientific) as well as anti-mouse CD8 (clone 53-6.7) conjugated to APC (Biolegend). Cells were analyzed by flow cytometry using an LSR Fortessa flow cytometer and the mean fluorescence intensity of PE within the CD8 positive population was used as a metric of TCR activation.

### In Vivo Inhibition of Allorei Ection

1×10⁶ MC38 cells (of C57BL/6 origin) were injected subcutaneously into the flank of ten BALB/cByJ mice at day zero and mice were divided in to two groups: a treatment group of five mice receiving LoSTIM and a control group of five mice receiving vehicle control (PBS). Beginning on day zero, mice in each group were injected intraperitoneally with either 200 µg of LoSTIM in 200 µL of PBS or 200 µL of PBS alone. These intraperitoneal injections were continued every three days. Tumor size was measured every three days with calipers in three dimensions.

### In Vivo Treatment of Syngeneic Tumor

2×10⁵ CT-26 cells were injected subcutaneously into the abdomen of BALB/cByJ mice at day 0. Tumor size was measured on day six and mice with measurable tumor were divided in three groups: a treatment group of five mice receiving LoSTIM, a control group of five mice receiving the anti-PD-1 parent antibody 29F.1A12, and a group of four mice receiving vehicle control (PBS). Beginning on day six, 200 µg of LoSTIM in 200 µL of PBS, 200 µg of 29F.1A12 in 200 µL of PBS, or 200 µL of PBS alone was injected intraperitoneally. These intraperitoneal injections were continued every three days. Mice were sacrificed when either the tumor size reached the predefined limit set by an IACUC-approved protocol or tumors became ulcerated. Tumor size was measured every three days with calipers in three dimensions until the first mouse was sacrificed.

### SE-UPLC Analysis and Preparative Size Exclusion Chromatography

For size exclusion ultra-performance liquid chromatography (SE-UPLC) analysis, 2 microliters of sample was injected into an ACQUITY UPLC Protein BEH SEC 200 with a 1.7 micrometer, 4.6 x 150 mm column with a flow of 0.3 mL/min for 10 minutes. A mobile phase of 50 mM sodium phosphate and 500 mM sodium chloride at pH 6.2 was used.

For preparative size exclusion chromatography, material was loaded on to a HiLoad 26/600 Superdex 200 column, which was run with a mobile phase of phosphate buffered saline at pH 7.4. Elution of protein was detected by measuring absorbance of UV light at 280 nm. Three peaks were detected, representing the three molecular species identified by SE-UPLC analysis. Elution fractions corresponding to these peaks were collected and pooled appropriately to yield three separate preparations, each corresponding to one of the three molecular species.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A bispecific molecule comprising a first part that specifically binds to PD-1 and a second part that specifically binds to a surface antigen of a target cell other than a T cell, wherein simultaneous binding of the bispecific molecule to the surface antigen and to PD-1 facilitates clustering of PD-1 in proximity to an immunological synapse and/or wherein simultaneous binding of the bispecific molecule to the surface antigen and to PD-1 reduces or prevents T cell activity, and/or toxicity directed to the target cell, optionally wherein
i) the bispecific molecule binds to PD-1 on a T cell;
ii) T cell activity is cytokine production and/or T cell proliferation;
iii) at least the first part or the second part comprises a monoclonal or polyclonal antibody, or an antigen-biding fragment thereof;
iv) the bispecific molecule has a single binding site for PD-1; and/or
v) the bispecific molecule has a single binding site for PD-1 and a single binding site for the surface antigen.

2. The bispecific molecule of claim 1, wherein i) the difference in the K_{d} of binding to PD-1 and the K_{d} of binding to the tissue-specific surface antigen is at least 10-fold, 50-fold, 100-fold, 500-fold, 1,000-fold, 5,000-fold, or 10,000-fold, optionally further wherein the K_{d} of binding to PD-1 is less than the K_{d} of binding to the tissue-specific surface antigen and/or ii) the difference in the off-rate when binding to PD-1 and the off-rate when binding to the tissue-specific surface antigen is at least 10-fold, 50-fold, 100-fold, 500-fold, 1,000 fold, 5,000-fold, or 10,000-fold, optionally further wherein the off-rate of binding to PD-1 is slower than the off-rate of binding to the tissue-specific surface antigen.

3. The bispecific molecule of claim 1 or 2, wherein the first part specifically binds to PD-1 at a site different from the PD-L1 binding site of PD-1.

4. The bispecific molecule of any one of claims 1 to 3, wherein the first part blocks binding of neither PD-L1 nor PD-L2 to PD-1.

5. The bispecific molecule of claim 3 or 4, wherein the bispecific molecule acts as an agonist of PD-1 at a cell or tissue expressing the surface antigen, but not at a cell or tissue not expressing the surface antigen.

6. The bispecific molecule of claim 1 or 2, wherein the first part i) specifically binds to PD-1 and blocks the binding of PD-1 with PD-L1 or PD-L2 and/or ii) specifically binds to PD-1 at the PD-L1 and/or PD-L2 binding site of PD-1.

7. The bispecific molecule of claim 5 or 6, wherein the bispecific molecule acts as an agonist of PD-1 at a cell or tissue expressing the surface antigen, and acts as an antagonist of PD-1 at a cell or tissue not expressing the surface antigen.

8. The bispecific molecule of claim 1 or 2, wherein the first part comprises an amino acid sequence having at least 60%, 65%, 70%, 75%, 80%, 85%, 90%, or 95% sequence identity with an amino acid sequence selected from the group consisting of sequences listed in Table 1, Table 4A, and Table 4B, optionally wherein the sequence is SEQ ID NO: 30.

9. The bispecific molecule of claim 1, 2, or 8, wherein the first part comprises an amino acid sequence having at least 60%, 65%, 70%, 75%, 80%, 85%, 90%, or 95% sequence identity with SEQ ID NO: 31.

10. The bispecific molecule of claim 1 or 2, wherein the first part comprises an amino acid sequence having at least 60%, 65%, 70%, 75%, 80%, 85%, 90%, or 95% sequence identity with an extracellular domain of PD-L1 or PD-L2, optionally wherein the target is a receptor for the extracellular domain and/or the extracellular domain of PD-L1 has the sequence of SEQ ID NO: 24.

11. The bispecific molecule of claim 1 or 2, wherein the surface antigen is selected from the group of surface antigens listed in Table 3A, Table 3B, and Table 3C, or an MHC class I allele and/or an MHC class II allele, optionally wherein the allele is an HLA allele or wherein the MHC allele is H-2Kb.

12. The bispecific molecule of claim 11, wherein the second part comprises an amino acid sequence having at least 60%, 65%, 70%, 75%, 80%, 85%, 90%, or 95% sequence identity with SEQ ID NO: 21.

13. The bispecific molecule of claim 11 or 12, wherein the second part comprises an amino acid sequence having at least 60%, 65%, 70%, 75%, 80%, 85%, 90%, or 95% sequence identity with SEQ ID NO: 22.

14. The bispecific molecule of claim 1 or 2, wherein the bispecific molecule comprises an amino acid sequence having at least 60%, 65%, 70%, 75%, 80%, 85%, 90%, or 95% sequence identity with any one of SEQ ID NOs: 1, 2, 3, 4, 5, 6, 7, 8, 9, 10, 11, 12, 13, and 14.

15. The bispecific molecule of any one of claims 1-14, wherein at least the first part or the second part is a chimeric, humanized, composite, murine, or human monoclonal or polyclonal antibody, or antigen-binding fragment thereof.

16. The bispecific molecule of any one of claims 1-15, wherein at least the first part or the second part is detectably labeled, comprises an effector domain, comprises an Fc domain, and/or is selected from the group consisting of Fv, Fav, F(ab′)2), Fab′, dsFv, scFv, sc(Fv)2, and diabodies fragments, optionally wherein the Fc domain is modified to decrease binding to Fc gamma receptor.

17. An isolated nucleic acid molecule that encodes a bispecific molecule of any one of claims 1-16, optionally wherein the isolated nucleic acid molecule hybridizes, under stringent conditions, with the complement of a nucleic acid encoding a polypeptide selected from the group consisting of the sequences of SEQ ID NOs: 1-14, 21-26, 28, and 30-31, or a sequence with at least about 95% homology to a nucleic acid encoding a polypeptide selected from the group consisting of the sequences of SEQ ID NOs: 1-14, 21-26, 28, and 30-31, optionally wherein the isolated nucleic acid molecule is a stabilized mRNA.

18. A vector comprising the isolated nucleic acid of claim 17.

19. A host cell which comprises the isolated nucleic acid of claim 17, comprises the vector of claim 18, or expresses the bispecific molecule of any one of claims 1-16, optionally wherein the host cell is genomically engineered and/or is a T cell that expresses a heterologous protein such as a chimeric antigen receptor (CAR).

20. A device or kit comprising at least one bispecific molecule according to any one of claims 1-16, said device or kit optionally comprising a label to detect the at least one bispecific molecule or a complex comprising the bispecific molecule.

21. A method of producing at least one bispecific molecule according to any one of claims 1-16, which method comprises the steps of: (i) culturing a transformed host cell which has been transformed by a nucleic acid comprising a sequence encoding at least one bispecific molecule according to any one of claims 1-16 under conditions suitable to allow expression of said bispecific molecule; and (ii) recovering the expressed bispecific molecule.

22. A method of inhibiting activation of T cells in a cell-specific and/or tissue-specific manner in a sample, comprising contacting the sample with the bispecific molecule of any one of claims 1-16.

23. The method of claim 22, wherein the bispecific molecule inhibits activation of T cells interacting with the target cell when the bispecific molecule simulataneously binds to the surface antigen and to PD-1.

24. A method of inhibiting activation of T cells in a cell-specific and/or tissue-specific manner in a subject, comprising administering to the subject the bispecific molecule of any one of claims 1-16, optionally wherein the subject is at risk of, suspected of having, or afflicted with a cancer.

25. The method of claim 24, wherein the bispecific molecule inhibits activation of T cells interacting with the target cell when the bispecific molecule simulataneously binds to the surface antigen and to PD-1.

26. The method of claim 25, wherein the subject is set to receive or has received solid organ transplantation.

27. The method of claim 26, wherein the surface antigen is a mismatched MHC class I or class II.

28. The method of claim 25, wherein the subject is at risk of, suspected of having, or afflicted with an autoimmune disease to thereby treat the autoimmune disease.

29. The method of claim 28, wherein the surface antigen is one that is expressed on pathologically inflamed tissue.

30. The method of claim 25, wherein the subject is at risk of, suspected of having, or afflicted with graft-versus-host disease (GVHD) to thereby treat the GVHD.

31. The method of claim 30, wherein the surface antigen is one that is expressed on inflamed tissue but not on cancerous cells and/or the subject is set to receive or has received an allogeneic bone marrow transplantation.

32. The method of claim 25, wherein the subject is at risk of, suspected of having, or afflited with checkpoint-inhibitor immune related adverse events (irAE) to thereby treat the irAE.

33. The method of claim 32, wherein the surface antigen is one that is expressed on inflamed tissue but not on cancerous cells.

34. The method of claim 25, wherein the subject is set to receive or has received an engineered immune cell therapy.

35. The method of claim 34, wherein the surface antigen is one that is expressed in normal tissues that also express a tumor-associated antigen targeted by the immune cell therapy, but is one that is not expressed on cancerous cells.
